(19) United States
(12) Patent Application Publication
(10) Pub. No.: US 2025/0127904 A1
(43) Pub. Date: Apr. 24, 2025

(54) SIGLEC LIGANDS, CONJUGATES, AND METHODS OF USE THEREOF

(71) Applicant: Osprey Biopharmaceuticals, Inc., San Diego, CA (US)

(72) Inventors: Francois Vermersch, San Diego, CA (US); Mehdi Numa, San Diego, CA (US); Grant Gabrelow, San Diego, CA (US)

(21) Appl. No.: 18/906,966

(22) Filed: Oct. 4, 2024

Related U.S. Application Data
(60) Provisional application No. 63/542,961, filed on Oct. 6, 2023.

(51) Int. Cl. A61K 47/54; A61P 37/06; C07D 309/02; C07D 407/02; C07K 16/42

(52) U.S. Cl. CPC A61K 47/545; A61P 37/06; C07D 309/02; C07D 407/02; C07K 16/42

(57) ABSTRACT

Provided are siglec ligands and siglec conjugates. Also provided are methods of use of such conjugates, as well as pharmaceutical compositions thereof.

### CROSS-REFERENCE TO RELATED APPLICATIONS

This application claims the benefit of U.S. Provisional Application No. 63/542,961, filed Oct. 6, 2023, the disclosure of which is incorporated by reference herein in its entirety.

### INTRODUCTION

Sialic acid-binding immunoglobulin-type lectins, better known as siglecs, are a family of cell surface proteins that bind sialic acid. A total of 14 siglecs are found in humans, 13 of which are found on overlapping cell types of the immune system and one (siglec-4) is found on myelinating cells in the nervous system (Gonzalez-Gil, A.; Schnaar, R. L., Siglec Ligands, Cells 2021, 10, 1260). Each siglec has an N-terminal V-type immunoglobulin domain (Ig domain) containing a sialoglycan which acts as the binding receptor for sialic acid. Different siglecs bind to distinct sialoglycan ligands to initiate molecular and cellular responses important to the function of the cells on which they are expressed (Gonzalez-Gil, A.; Schnaar, R. L., Siglec Ligands, Cells 2021, 10, 1260). Knowing each siglec and its particular ligand provides understanding of cell physiology and pathology and introduces potential opportunities for therapeutic intervention.

Autoimmune disorders are caused when the immune system destroys native tissues, cells, or biomolecules by, e.g., mounting an immune response to self-antigens. Immune disorders are often treated with systemic immune suppression, e.g., by administering antibodies, steroids, gene therapy, etc. Such treatments are not always target specific and/or effective, resulting in compromised immune responses and side effects. Such treatments, when effective, may only be effective transiently.

Therapeutic proteins and gene therapies are novel and successful drug modalities for the treatment of disease. However, patient immune responses to such therapeutics often result in inhibition of drug activity, accelerated drug clearance, compromised drug safety, and loss of drug efficacy. Prevention of the formation of neutralizing and non-neutralizing drug-specific antibodies (“anti-drug antibodies” or “ADA”) is a key unsolved problem in the field of biotherapeutics. Blocking ADA responses to biotherapeutics would improve drug exposure, improve durability of efficacy, reduce ADA-related toxicities, and enable favorable pharmacology for otherwise undruggable modalities (e.g., de novo designed drugs, drugs based on endogenous proteins).

### SUMMARY

Provided are siglec ligands and siglec conjugates. Also provided are methods of use of such conjugates, as well as pharmaceutical compositions thereof.

### BRIEF DESCRIPTION OF THE DRAWINGS

FIGS. 1A and 1B show NMR spectroscopy and mass spectroscopy of intermediate A according to embodiments of the present disclosure.

FIGS. 2A and 2B show NMR spectroscopy and mass spectroscopy of intermediate B according to embodiments of the present disclosure.

FIGS. 3A and 3B show NMR spectroscopy and mass spectroscopy of intermediate C according to embodiments of the present disclosure.

FIGS. 4A and 4B show NMR spectroscopy and mass spectroscopy of compound 1 according to embodiments of the present disclosure.

FIGS. 5A and 5B show NMR spectroscopy and mass spectroscopy of compound 2 according to embodiments of the present disclosure.

FIGS. 6A and 6B show NMR spectroscopy and mass spectroscopy of compound 3 according to embodiments of the present disclosure.

FIGS. 7A and 7B show NMR spectroscopy and mass spectroscopy of compound 4 according to embodiments of the present disclosure.

FIGS. 8A and 8B show NMR spectroscopy and mass spectroscopy of compound 5 according to embodiments of the present disclosure.

FIGS. 9A and 9B show NMR spectroscopy and mass spectroscopy of compound 6 according to embodiments of the present disclosure.

FIGS. 10A and 10B show NMR spectroscopy and mass spectroscopy of compound 7 according to embodiments of the present disclosure.

FIGS. 11A and 11B show NMR spectroscopy and mass spectroscopy of compound 8 according to embodiments of the present disclosure.

FIGS. 12A and 12B show NMR spectroscopy and mass spectroscopy of compound 9 according to embodiments of the present disclosure.

FIGS. 13A and 13B show NMR spectroscopy and mass spectroscopy of compound 10 according to embodiments of the present disclosure.

FIGS. 14A and 14B show NMR spectroscopy and mass spectroscopy of compound 11 according to embodiments of the present disclosure.

FIGS. 15A and 15B show NMR spectroscopy and mass spectroscopy of compound 12 according to embodiments of the present disclosure.

FIGS. 16A and 16B show NMR spectroscopy and mass spectroscopy of compound 13 according to embodiments of the present disclosure.

FIGS. 17A and 17B show NMR spectroscopy and mass spectroscopy of compound 14 according to embodiments of the present disclosure.

FIGS. 18A and 18B show NMR spectroscopy and mass spectroscopy of compound 15 according to embodiments of the present disclosure.

FIGS. 19A and 19B show NMR spectroscopy and mass spectroscopy of compound 16 according to embodiments of the present disclosure.

FIG. 20 show liquid chromatography and mass spectroscopy of compound 17 according to embodiments of the present disclosure.

FIGS. 21A and 21B show NMR spectroscopy and mass spectroscopy of compound 18 according to embodiments of the present disclosure.

FIGS. 22A and 22B show NMR spectroscopy and mass spectroscopy of compound 20 according to embodiments of the present disclosure.

FIG. 23 shows liquid chromatography and mass spectroscopy of compound 21 according to embodiments of the present disclosure.

FIGS. 24A and 24B show NMR spectroscopy and mass spectroscopy compound 22 according to embodiments of the present disclosure.

FIG. 25 shows liquid chromatography and mass spectroscopy of compound 23 according to embodiments of the present disclosure.

FIG. 26A, FIG. 26B, FIG. 26C and FIG. 26D all depict in vitro B cell activation assays where mouse primary B cells were treated with either a B cell receptor-agonizing anti-lgD antibody or B cell receptor-agonizing anti-IgD-Siglec Ligand conjugates bearing monovalent or multivalent ligand structures. The B cell stimulatory activities of anti-IgD and anti-IgD-Siglec Ligand conjugates were compared in dose titration experiments with an activation readout of CD69 upregulation. CD69 levels on the different treatment groups were evaluated through the percentage of cells that were CD69-positive (FIG. 26A, and FIG. 26C) or the CD69 mean fluorescence intensity (MFI) (FIG. 26B and FIG. 26D).

FIGS. 27A, 27B and 27C show liquid chromatography and mass spectroscopy of compound 19 according to embodiments of the present disclosure.

FIGS. 28A and 28B show liquid chromatography and mass spectroscopy of compound 24 according to embodiments of the present disclosure.

FIGS. 29A and 29B show liquid chromatography and mass spectroscopy of compound 25 according to embodiments of the present disclosure.

FIGS. 30A, 30B and 30C show liquid chromatography and mass spectroscopy of compound 26 according to embodiments of the present disclosure.

FIGS. 31A and 31B show liquid chromatography and mass spectroscopy of compound 27 according to embodiments of the present disclosure.

FIGS. 32A and 32B show liquid chromatography and mass spectroscopy of compound 28 according to embodiments of the present disclosure.

FIGS. 33A and 33B show liquid chromatography and mass spectroscopy of compound 29 according to embodiments of the present disclosure.

FIGS. 34A and 34B show liquid chromatography and mass spectroscopy of compound 30 according to embodiments of the present disclosure.

FIGS. 35A, 35B and 35C show liquid chromatography and mass spectroscopy of compound 31 according to embodiments of the present disclosure.

FIGS. 36A, 36B and 36C show liquid chromatography and mass spectroscopy of compound 32 according to embodiments of the present disclosure.

FIGS. 37A, 37B and 37C show liquid chromatography and mass spectroscopy of compound 33 according to embodiments of the present disclosure.

FIGS. 38A and 38B show liquid chromatography and mass spectroscopy of compound 34 according to embodiments of the present disclosure.

FIGS. 39A and 39B show liquid chromatography and mass spectroscopy of compound 35 according to embodiments of the present disclosure.

FIGS. 40A, 40B and 40C show liquid chromatography and mass spectroscopy of compound 36 according to embodiments of the present disclosure.

FIGS. 41A and 41B show liquid chromatography and mass spectroscopy of compound 37 according to embodiments of the present disclosure.

FIGS. 42A and 42B show liquid chromatography and mass spectroscopy of compound 38 according to embodiments of the present disclosure.

FIGS. 43A and 43B show liquid chromatography and mass spectroscopy of compound 39 according to embodiments of the present disclosure.

FIGS. 44A and 44B show liquid chromatography and mass spectroscopy of compound 40 according to embodiments of the present disclosure.

FIGS. 45A and 45B show liquid chromatography and mass spectroscopy of compound 41 according to embodiments of the present disclosure.

FIGS. 46A and 46B show liquid chromatography and mass spectroscopy of compound 42 according to embodiments of the present disclosure.

FIGS. 47A and 47B show liquid chromatography and mass spectroscopy of compound 43 according to embodiments of the present disclosure.

FIGS. 48A and 48B show liquid chromatography and mass spectroscopy of compound 44 according to embodiments of the present disclosure.

FIG. 49 shows liquid chromatography and mass spectroscopy of compound 45 according to embodiments of the present disclosure.

FIGS. 50A and 50B show liquid chromatography and mass spectroscopy of compound 46 according to embodiments of the present disclosure.

FIGS. 51A and 51B show liquid chromatography and mass spectroscopy of compound 47 according to embodiments of the present disclosure.

FIGS. 52A and 52B show liquid chromatography and mass spectroscopy of compound 48 according to embodiments of the present disclosure.

FIGS. 53A and 53B show liquid chromatography and mass spectroscopy of compound 49 according to embodiments of the present disclosure.

FIGS. 54A and 54B show liquid chromatography and mass spectroscopy of compound 50 according to embodiments of the present disclosure.

FIGS. 55A and 55B show liquid chromatography and mass spectroscopy of compound 51 according to embodiments of the present disclosure.

FIGS. 56A and 56B show liquid chromatography and mass spectroscopy of compound 52 according to embodiments of the present disclosure.

FIGS. 57A and 57B show liquid chromatography and mass spectroscopy of compound 53 according to embodiments of the present disclosure.

FIGS. 58A and 58B show liquid chromatography and mass spectroscopy of compound 54 according to embodiments of the present disclosure.

FIGS. 59A and 59B show liquid chromatography and mass spectroscopy of compound 55 according to embodiments of the present disclosure.

FIGS. 60A and 60B show liquid chromatography and mass spectroscopy of compound 56 according to embodiments of the present disclosure.

FIGS. 61A and 61B show liquid chromatography and mass spectroscopy of compound 57 according to embodiments of the present disclosure.

FIGS. 62A and 62B describe measurement of the Siglec receptor binding specificity of Siglec ligands. FIG. 62A describes the method for the flow cytometry-based competitive Siglec selectivity assay where biotinylated CD22 ligand is immobilized on a streptavidin bead. A fixed concentration of Human Siglec-2-Fc-AF647 is then queried for competitive binding to CD22 ligand-bead. FIG. 62B shows the results of the competitive binding experiment at high concentration of Siglec protein competitor.

FIG. 63A demonstrates the suppressive effects on human B cell activation with anti-human-IgM CD22 ligand conjugates on primary human PBMC-derived B cells. FIG. 63B shows the competitive cell binding of anti-IgM-CD22 ligand conjugates in comparison to unmodified anti-IgM antibody.

FIG. 64A shows the ADA titers on day 20 of mice dosed at 4 mg/kg with adalimumab control versus CD22 ligand conjugated adalimumab. FIG. 64B depicts the clearance rates of adalimumab control versus CD22 ligand conjugated adalimumab.

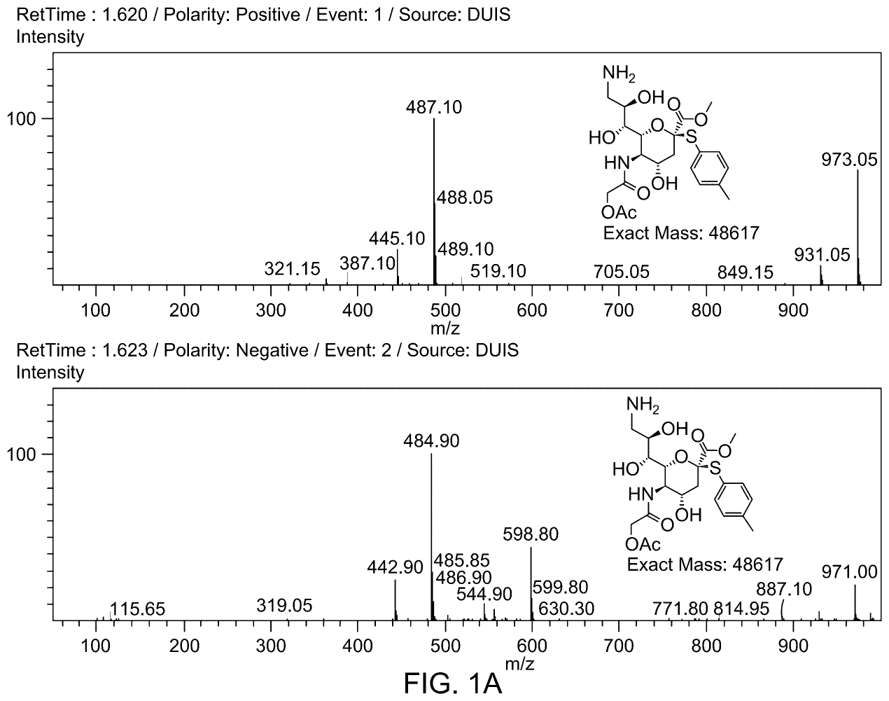

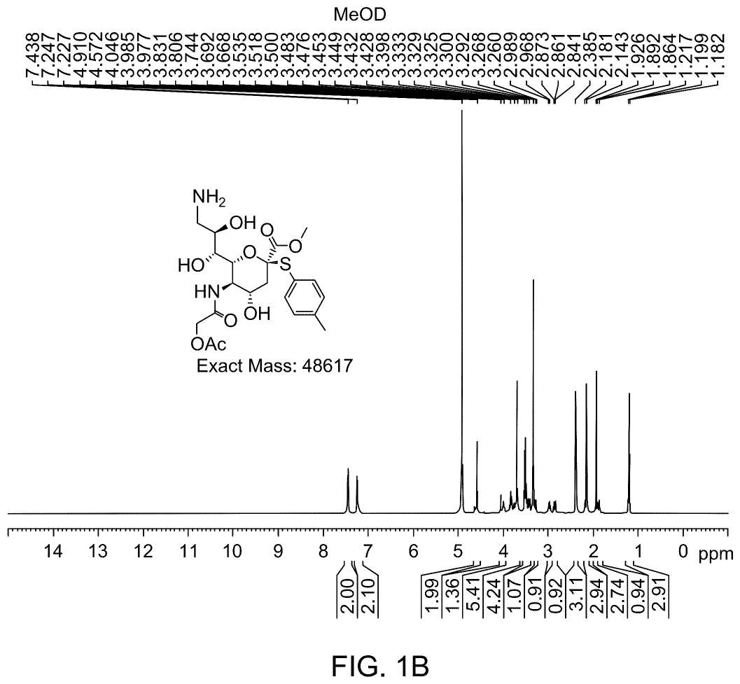

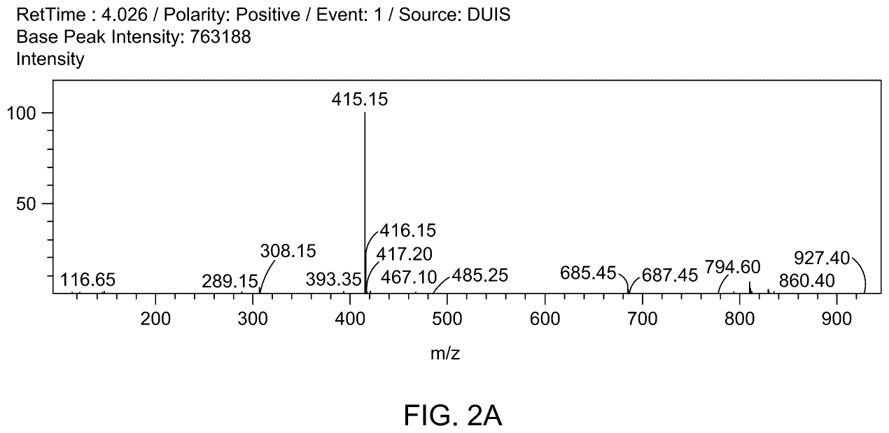

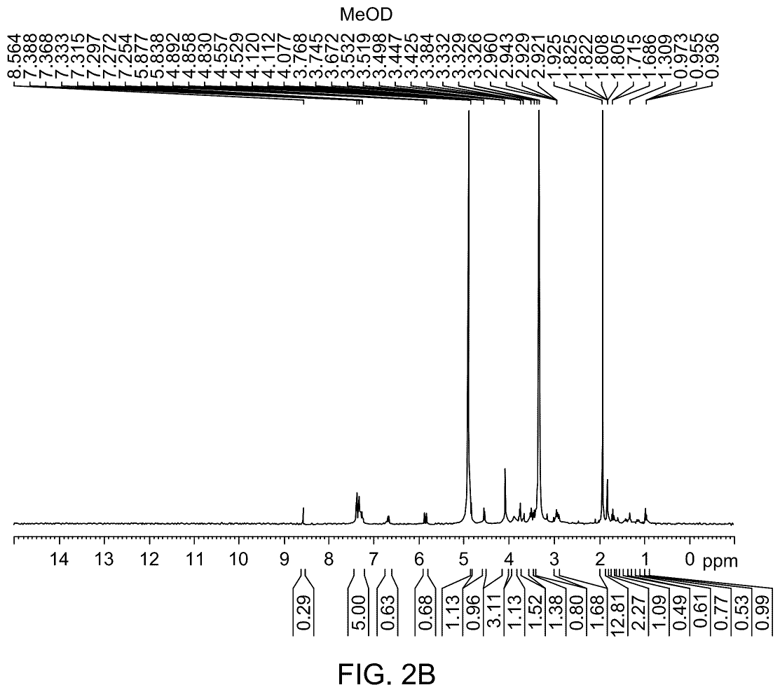

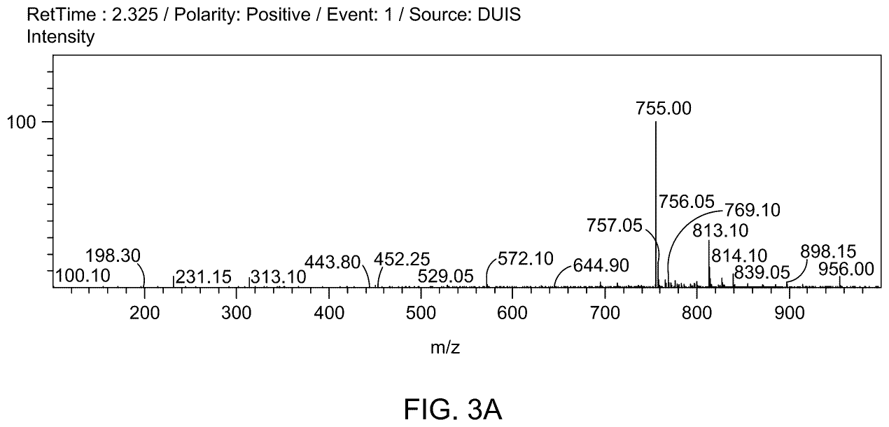

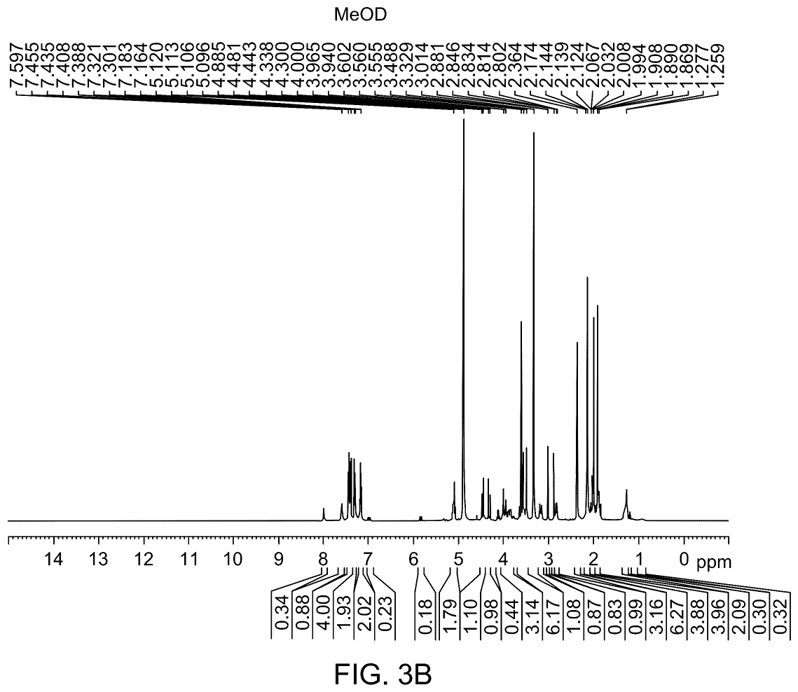

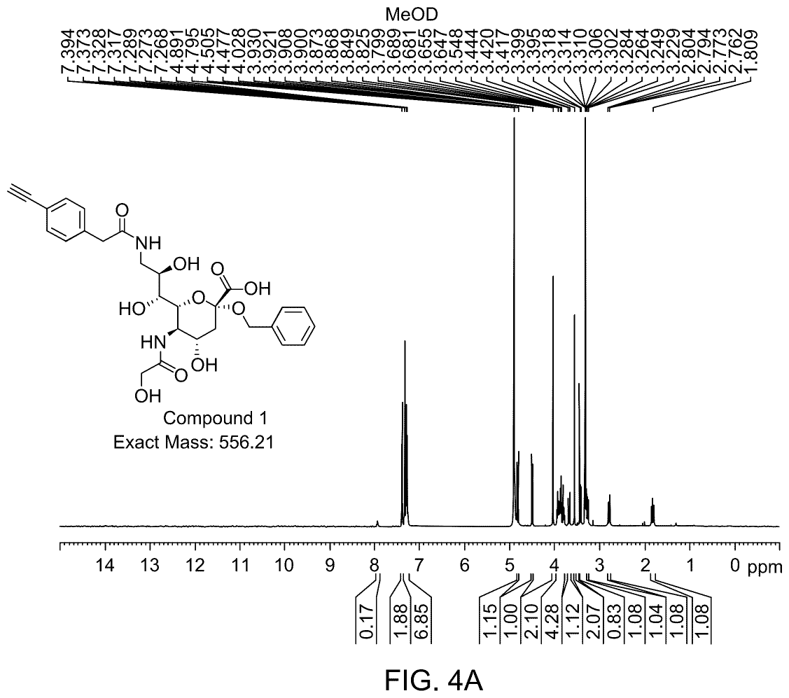

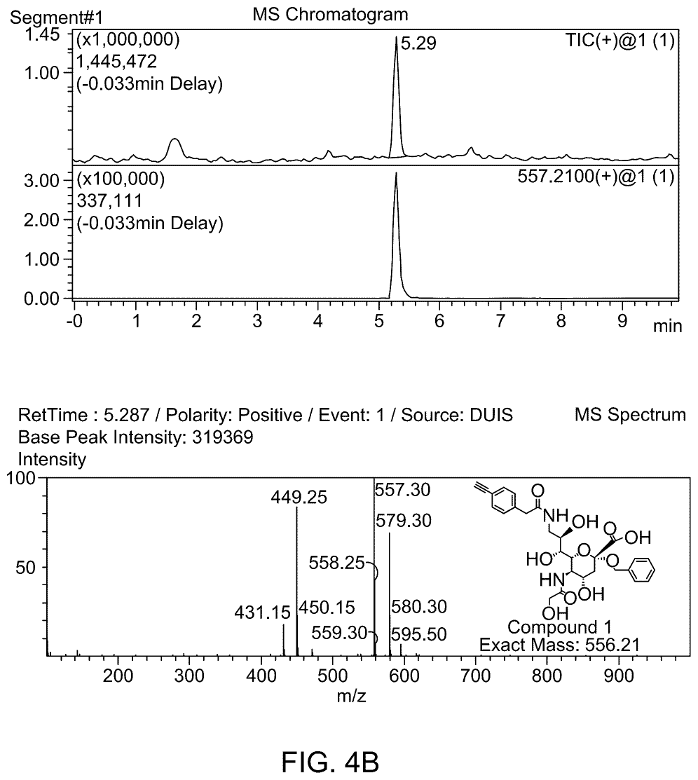

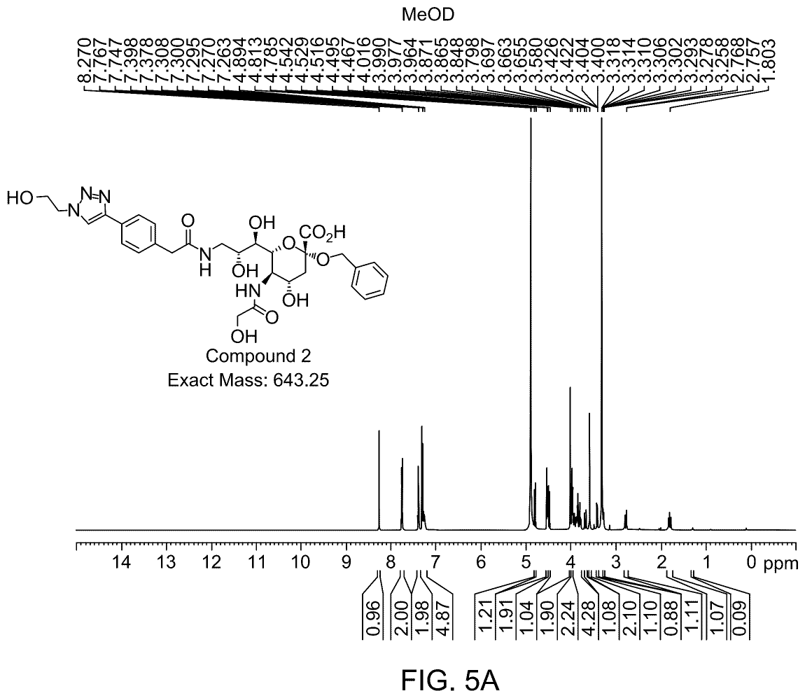

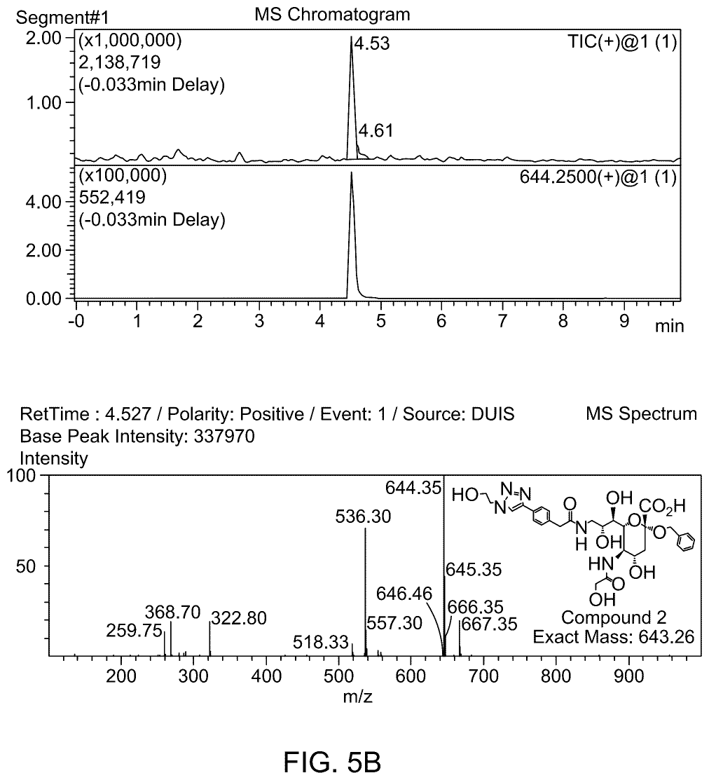

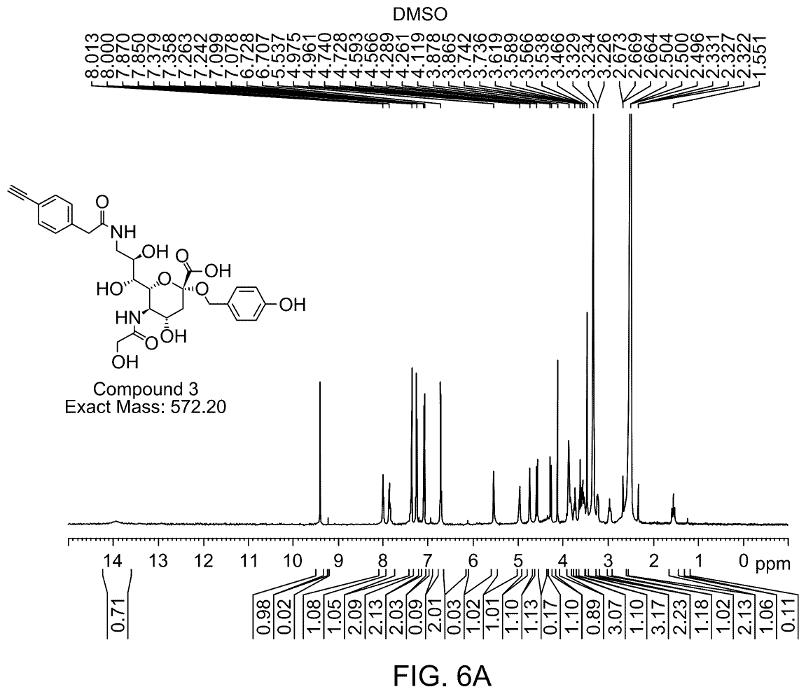

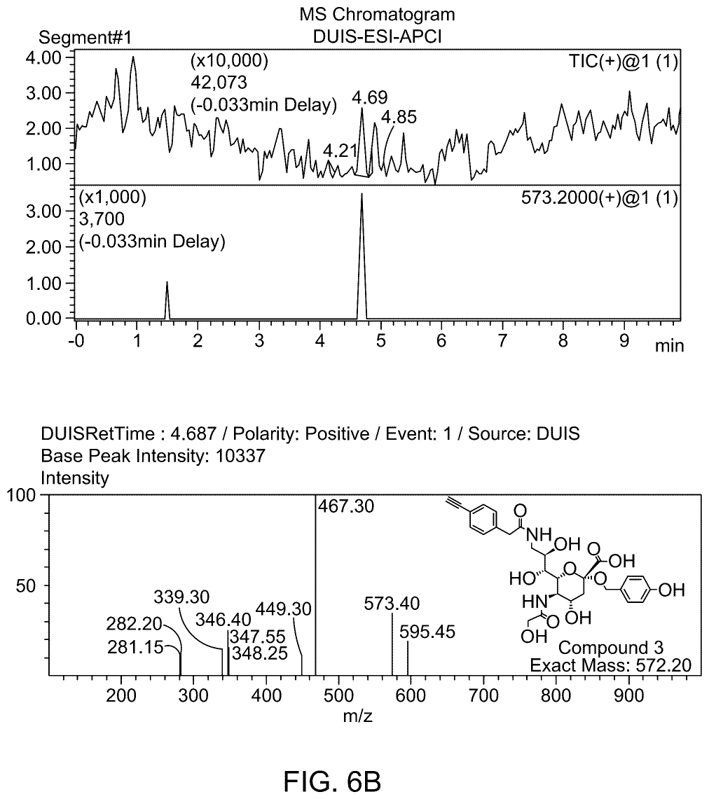

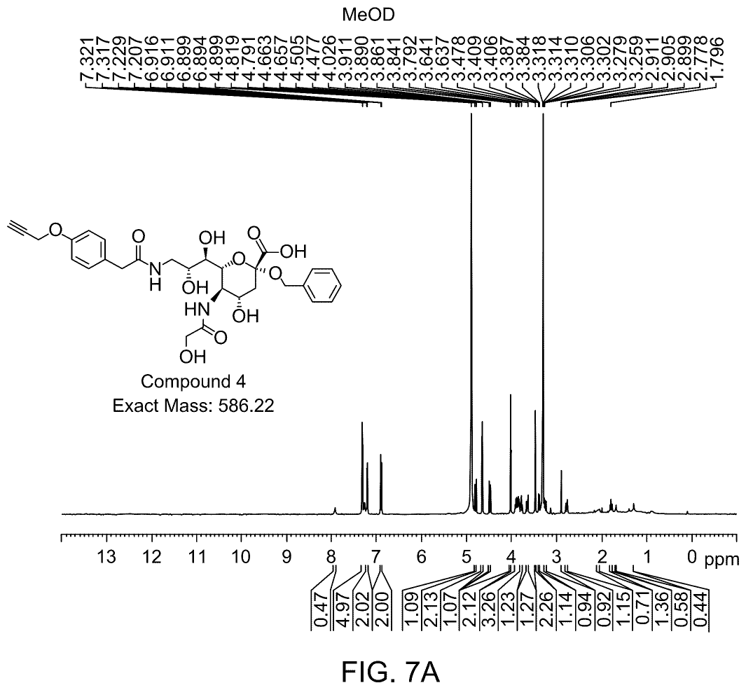

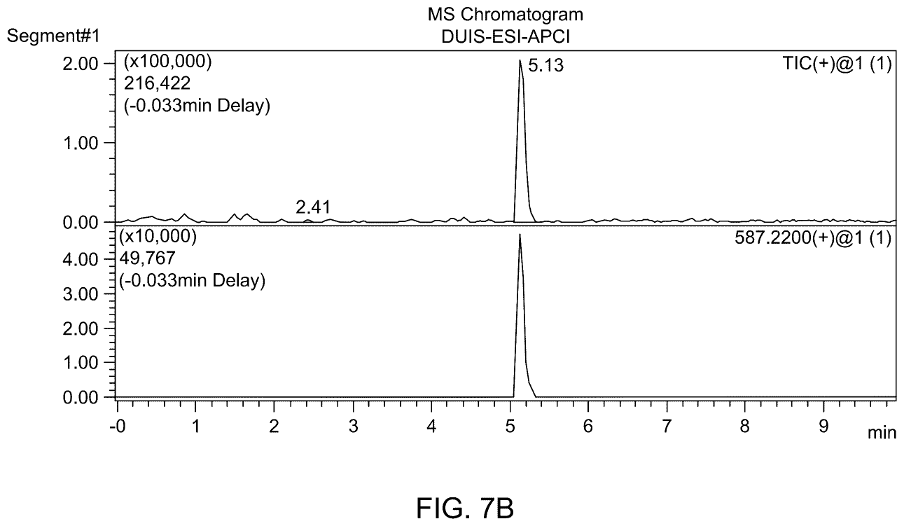

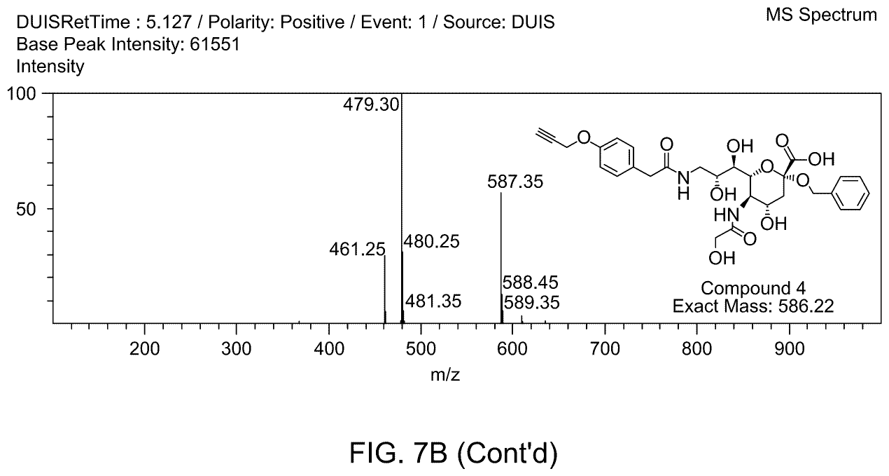

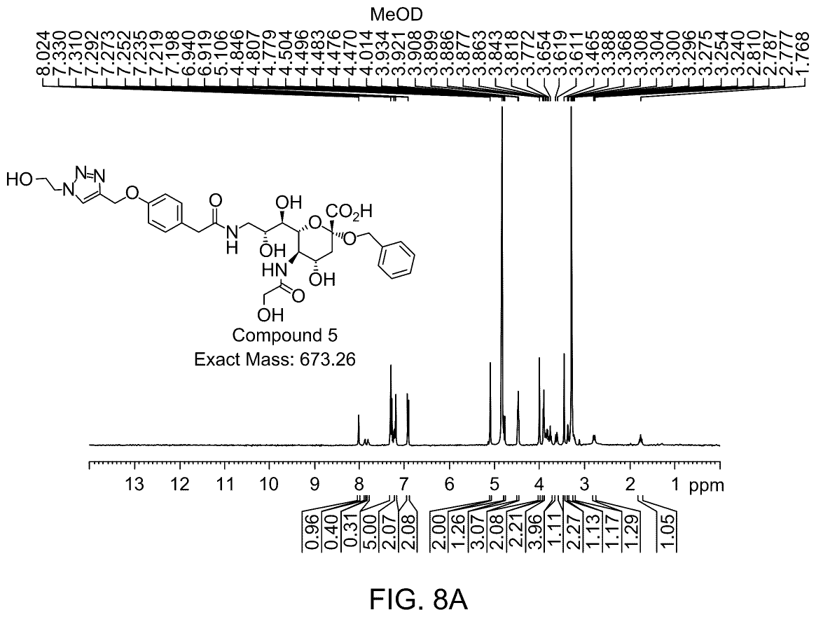

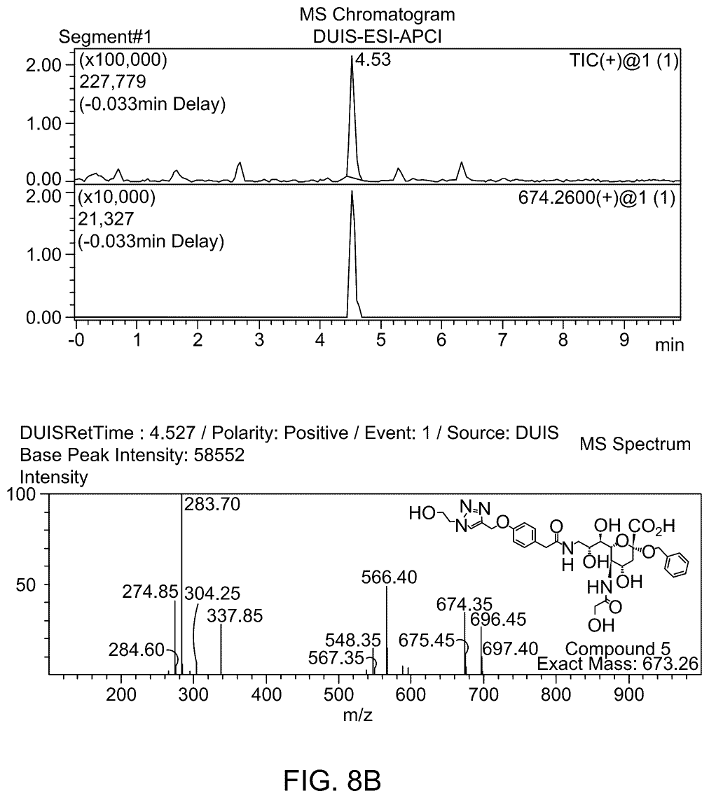

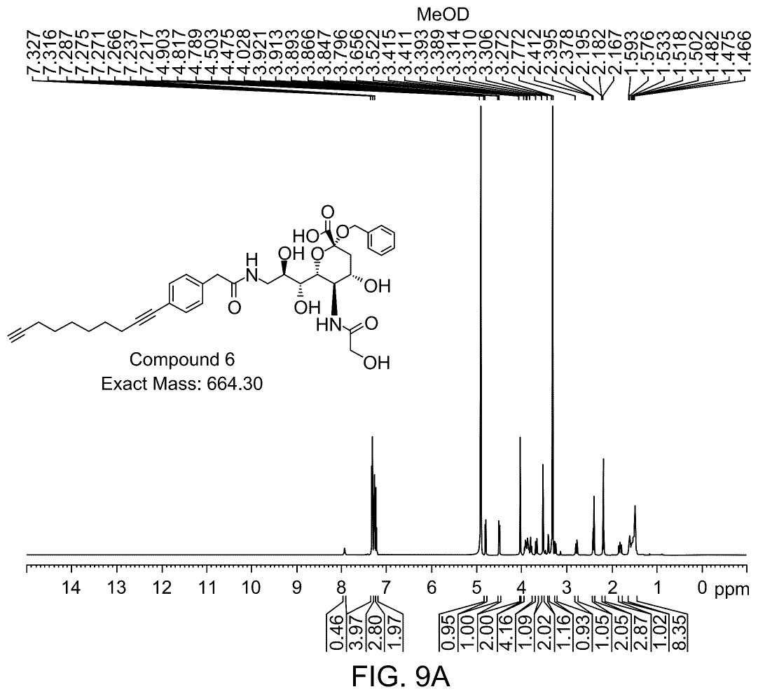

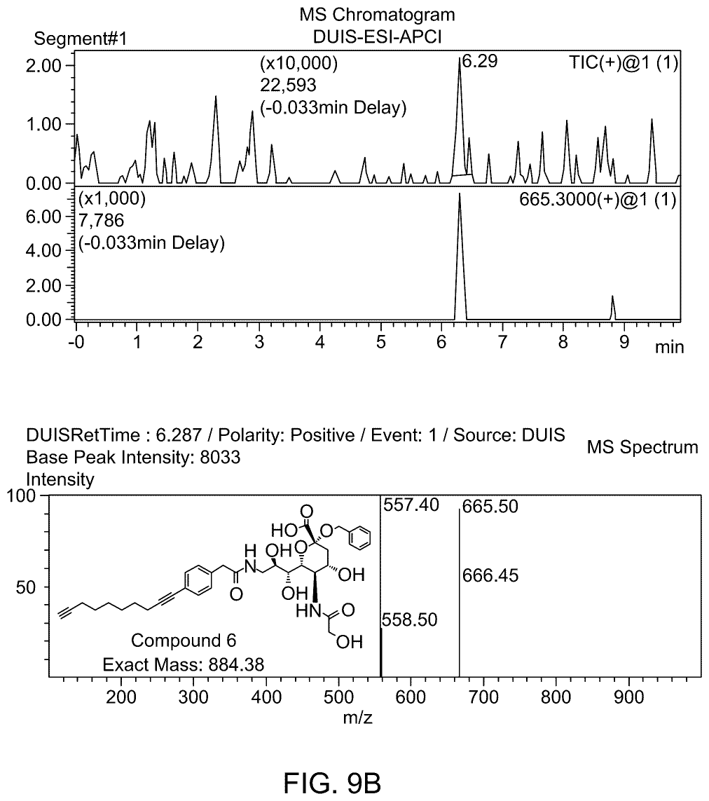

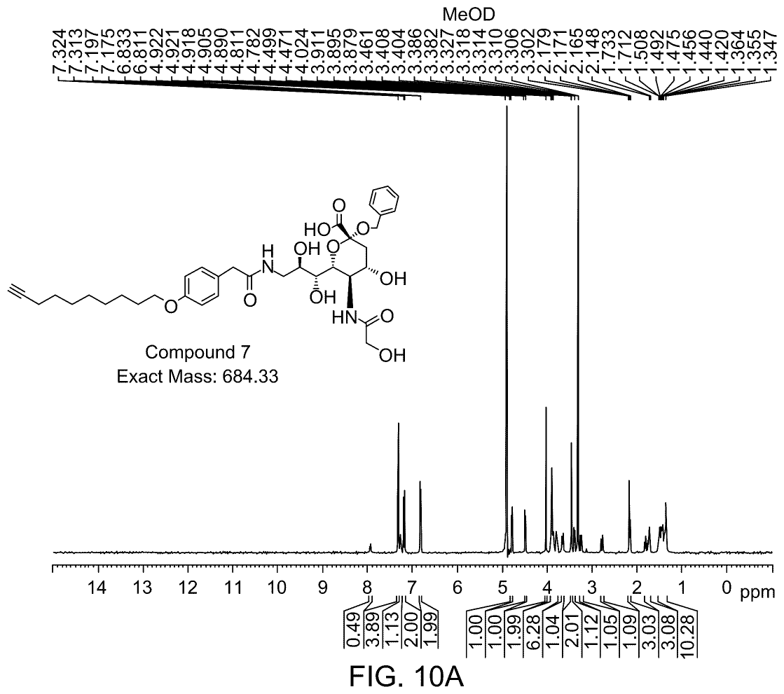

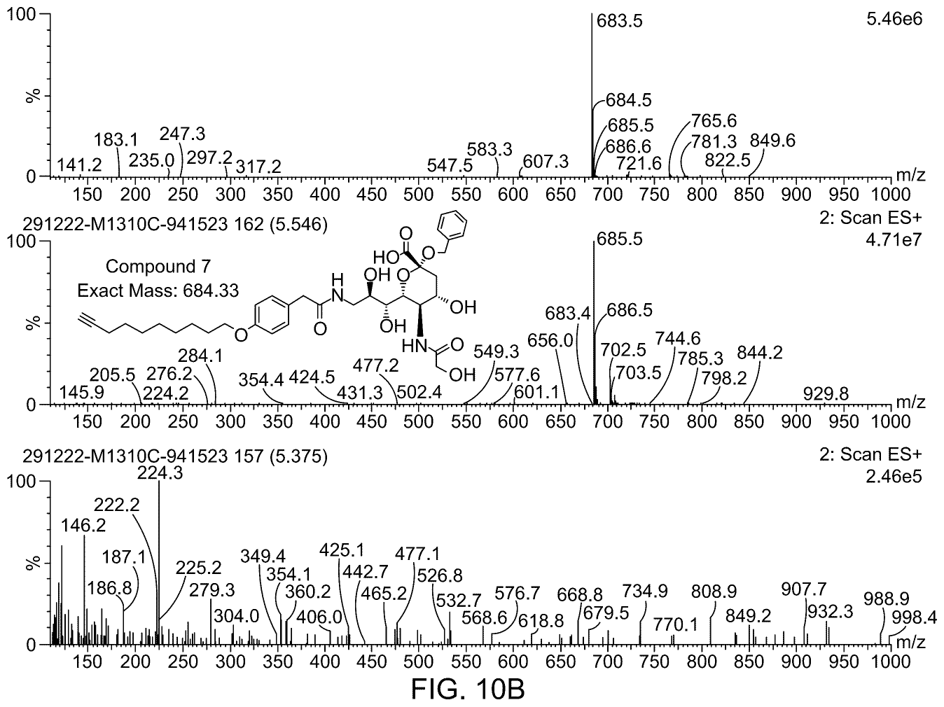

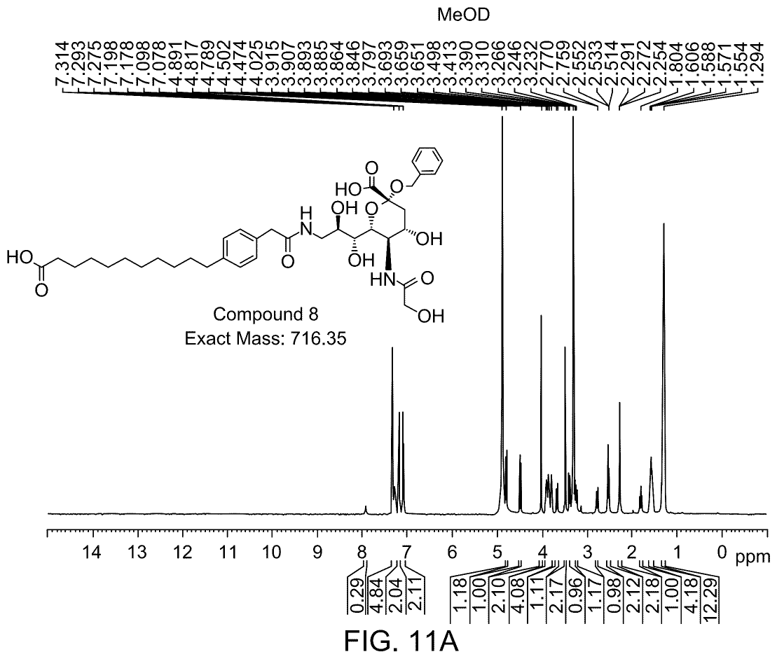

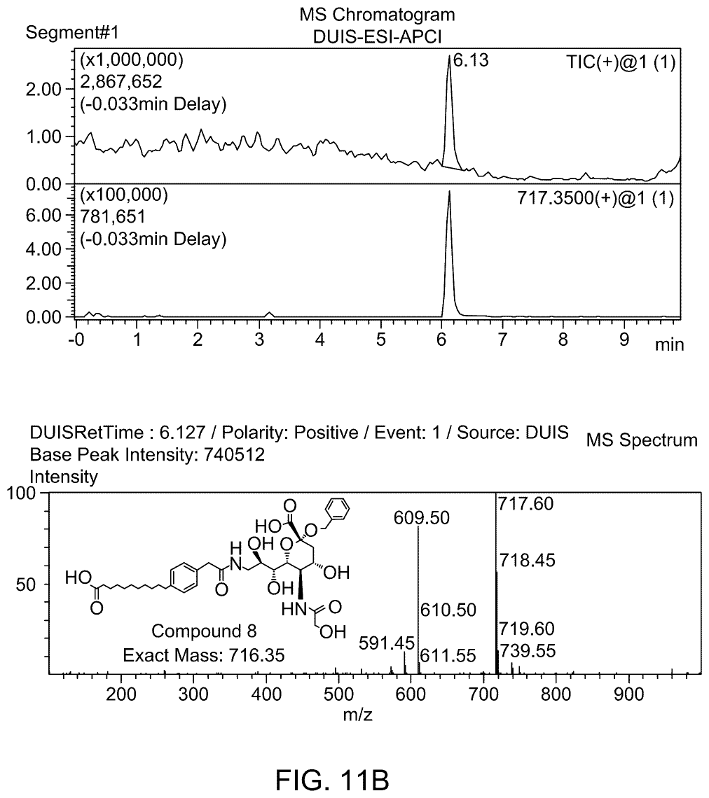

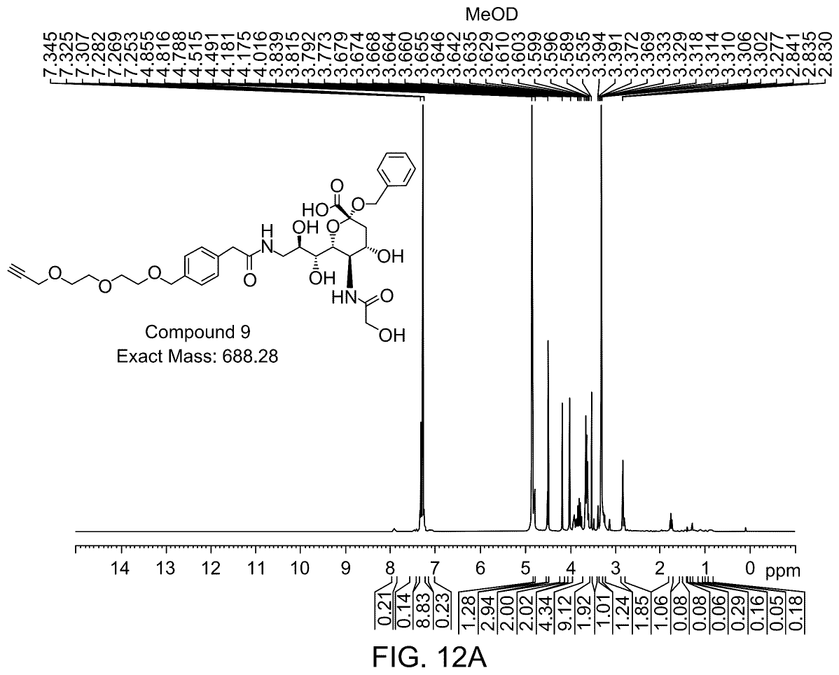

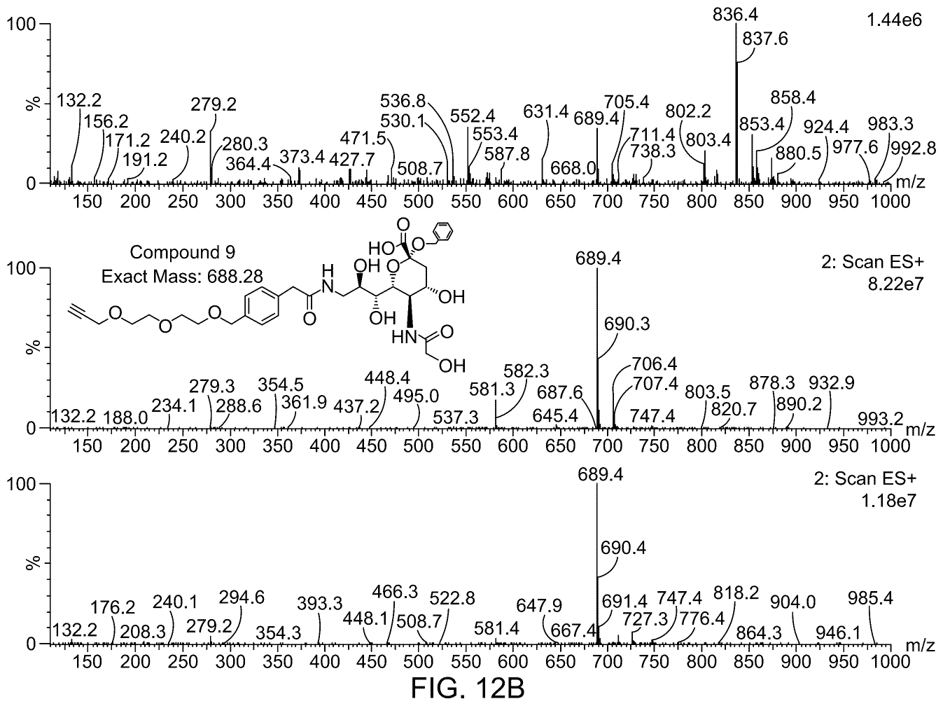

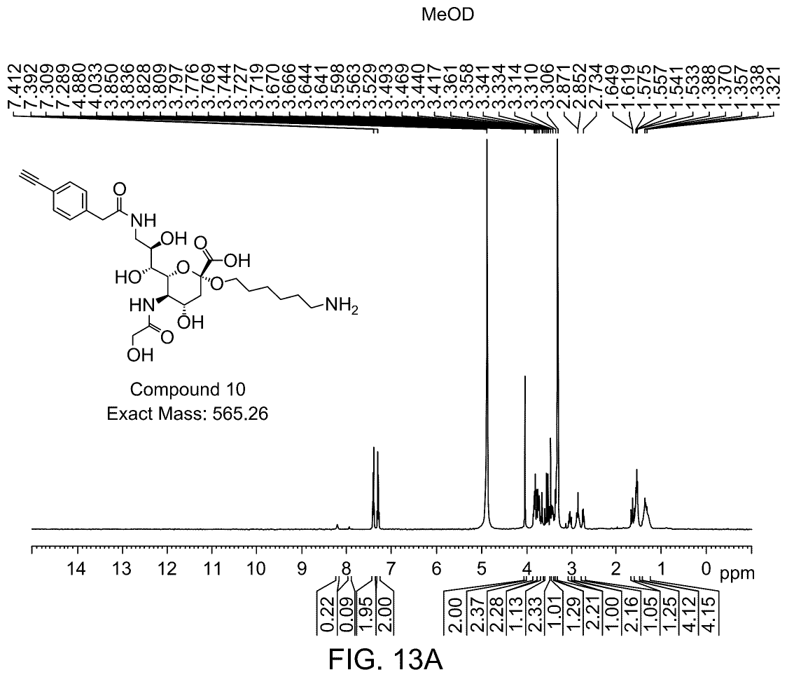

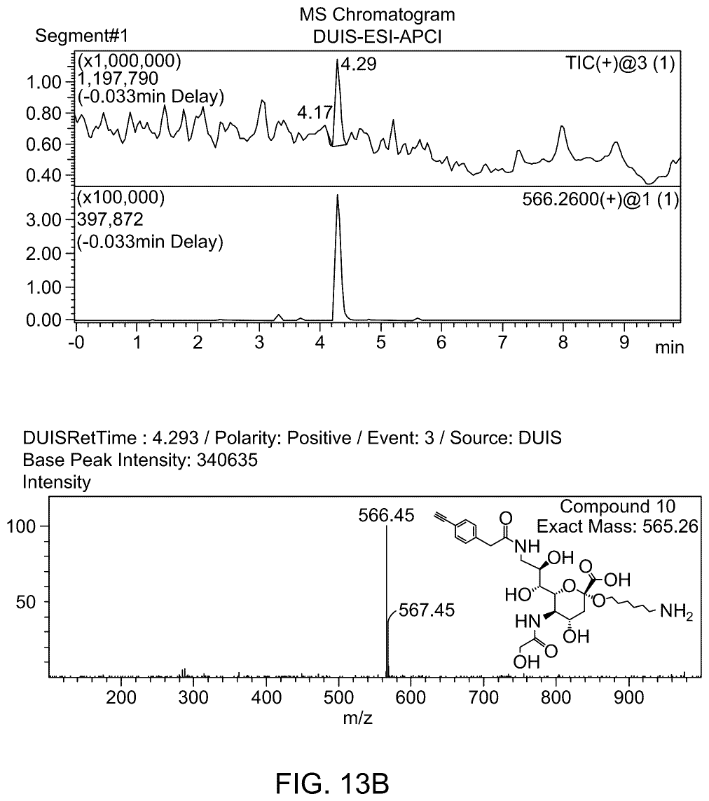

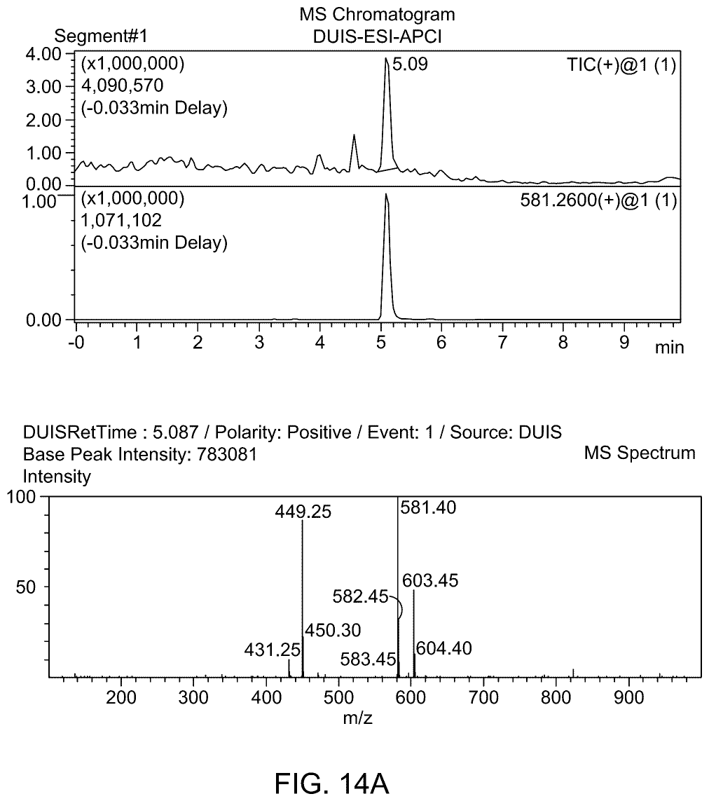

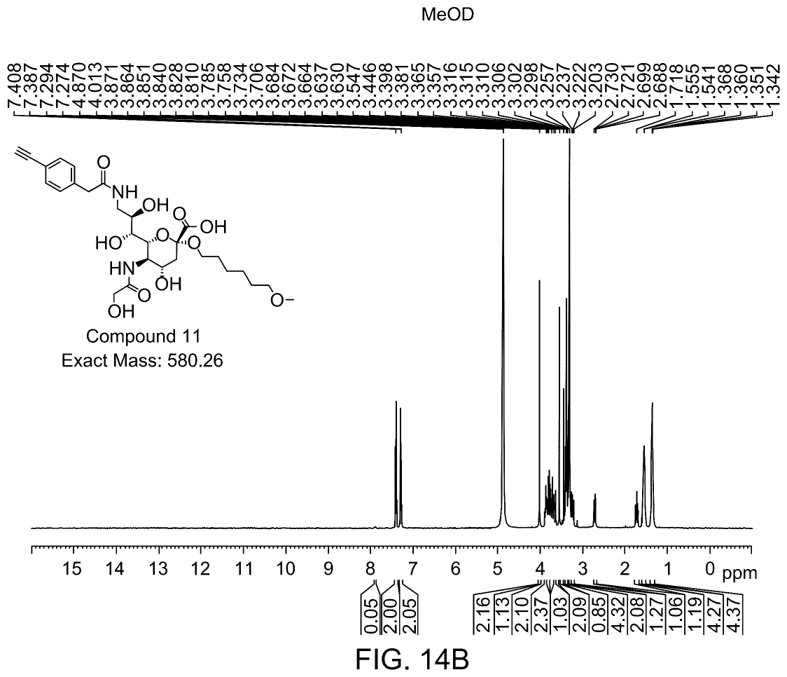

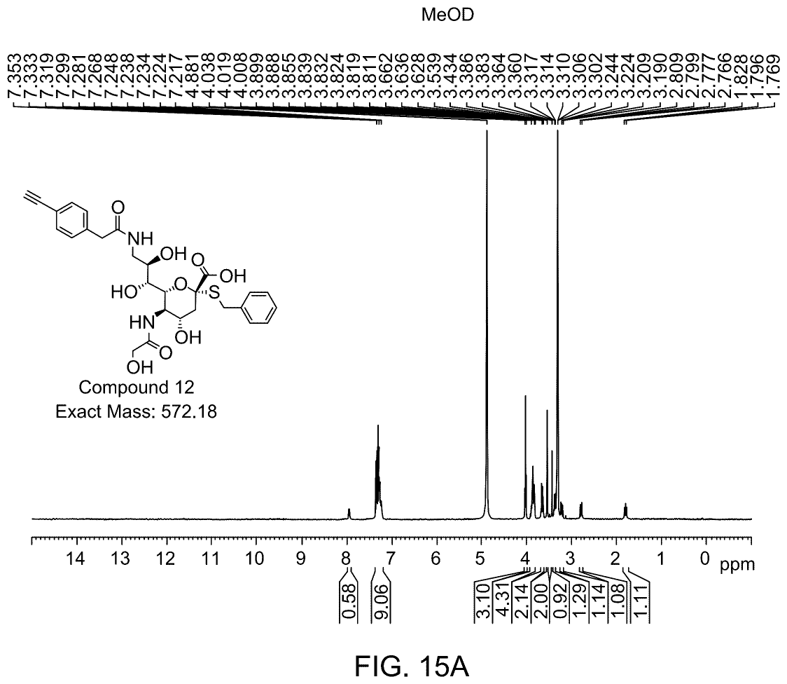

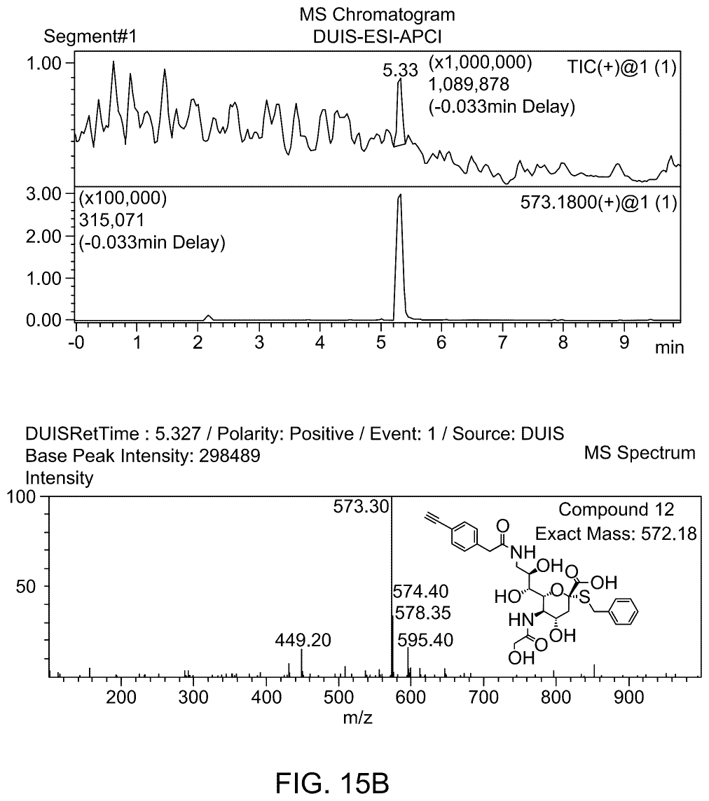

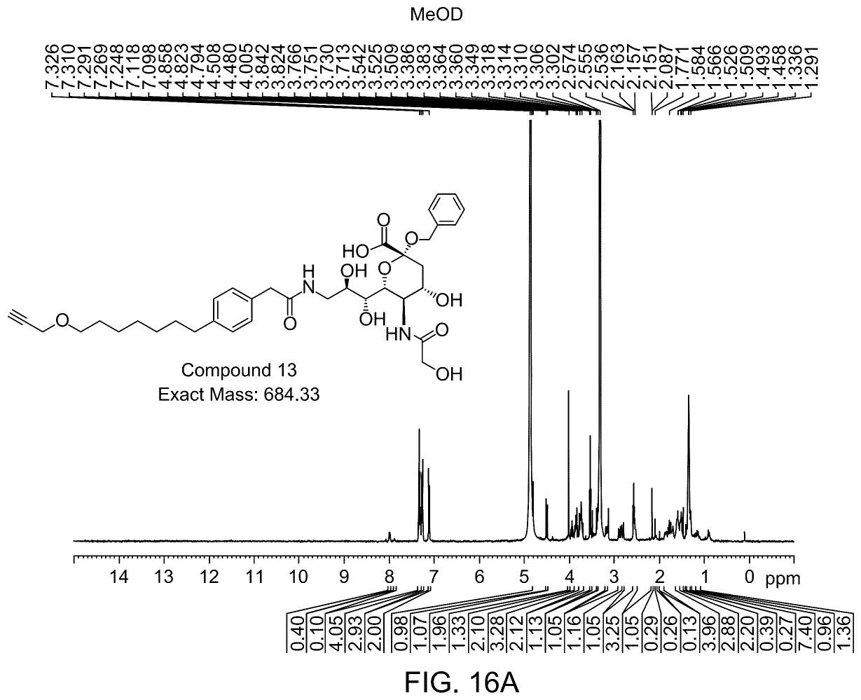

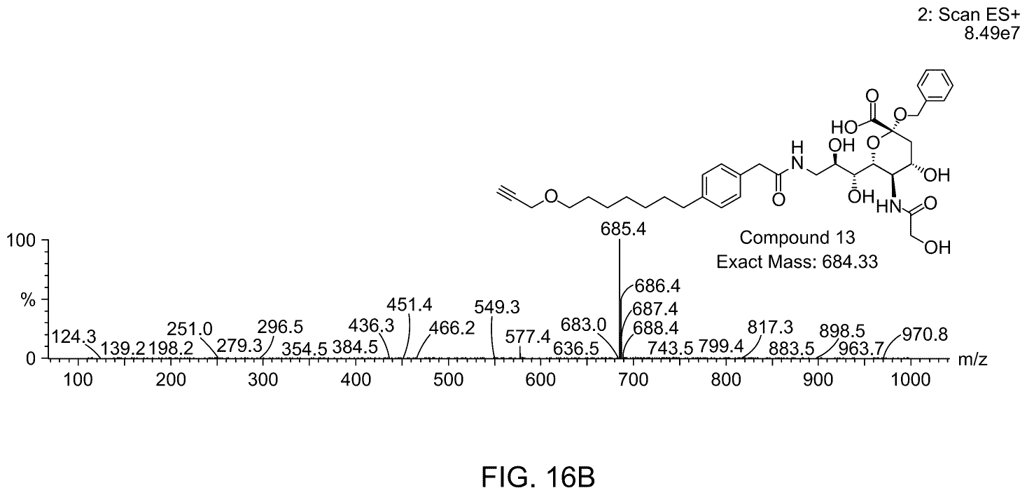

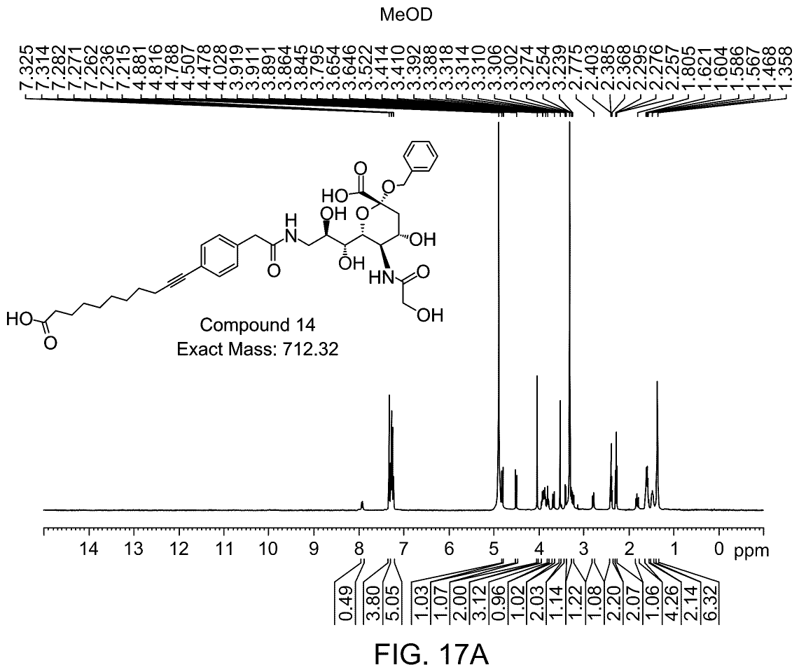

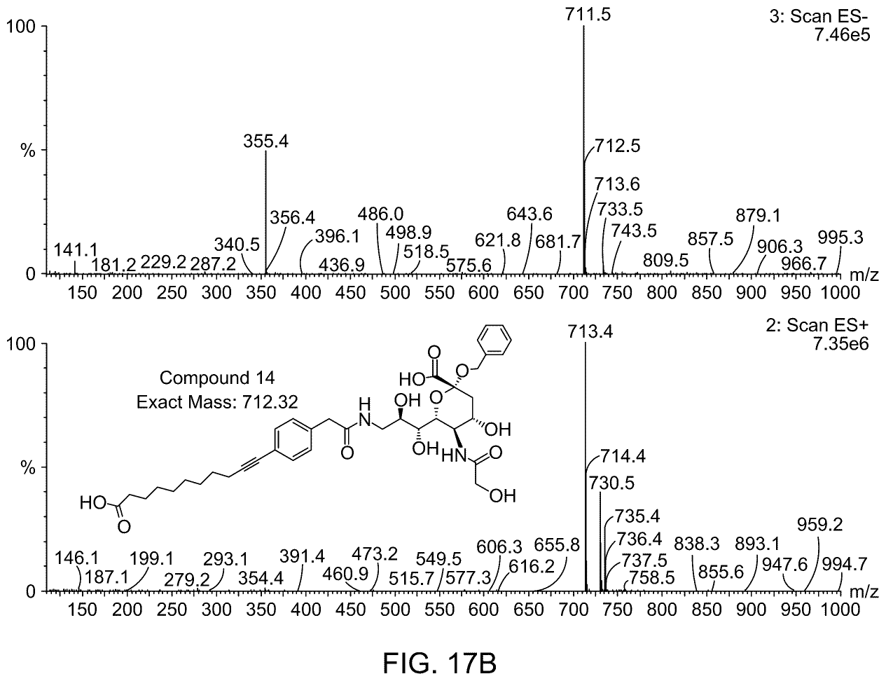

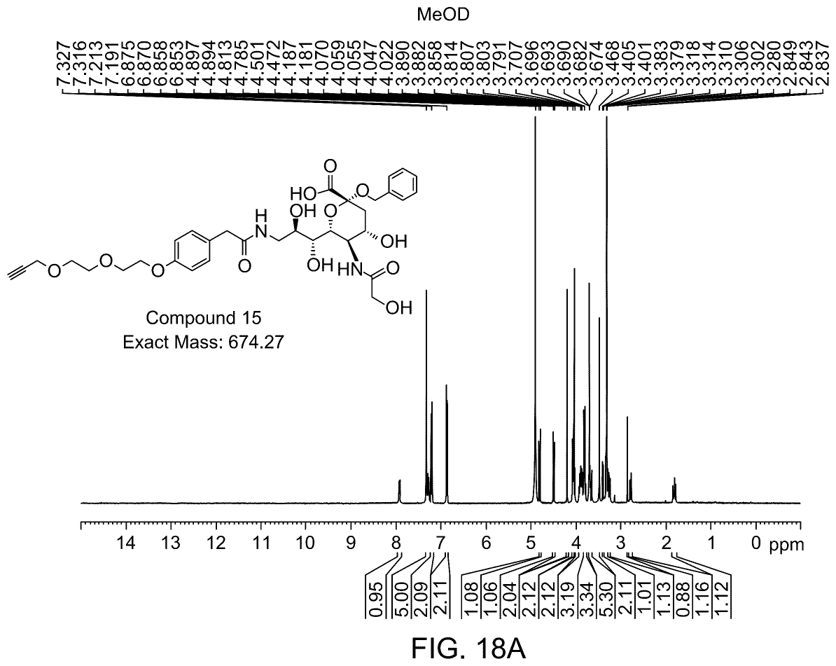

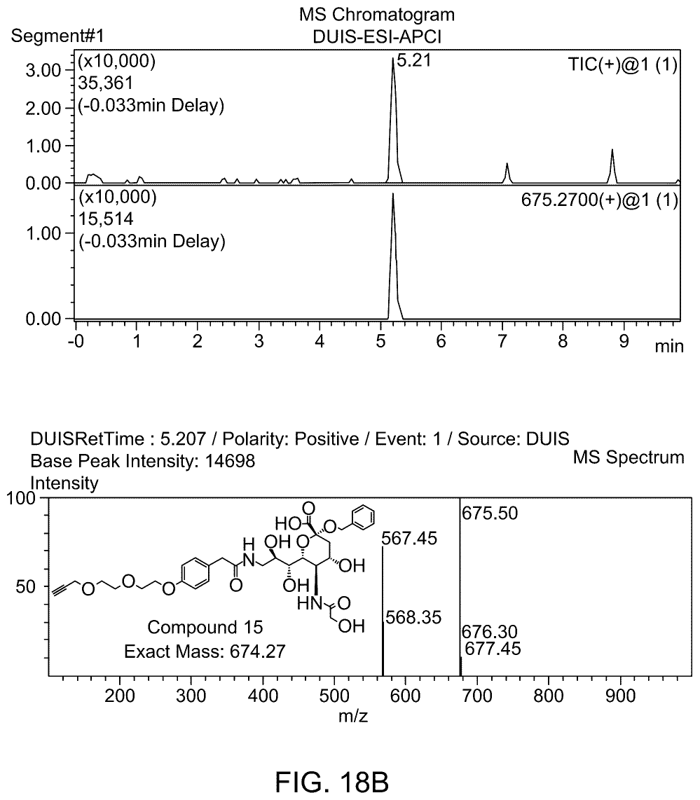

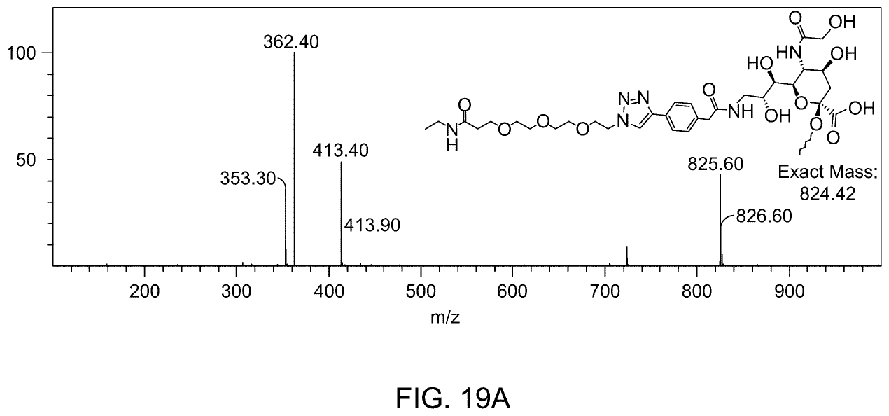

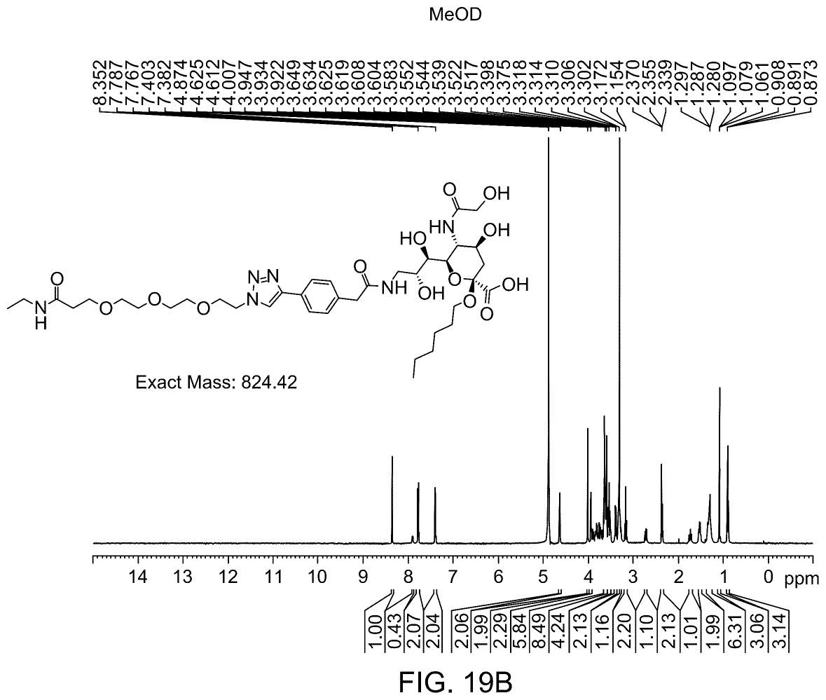

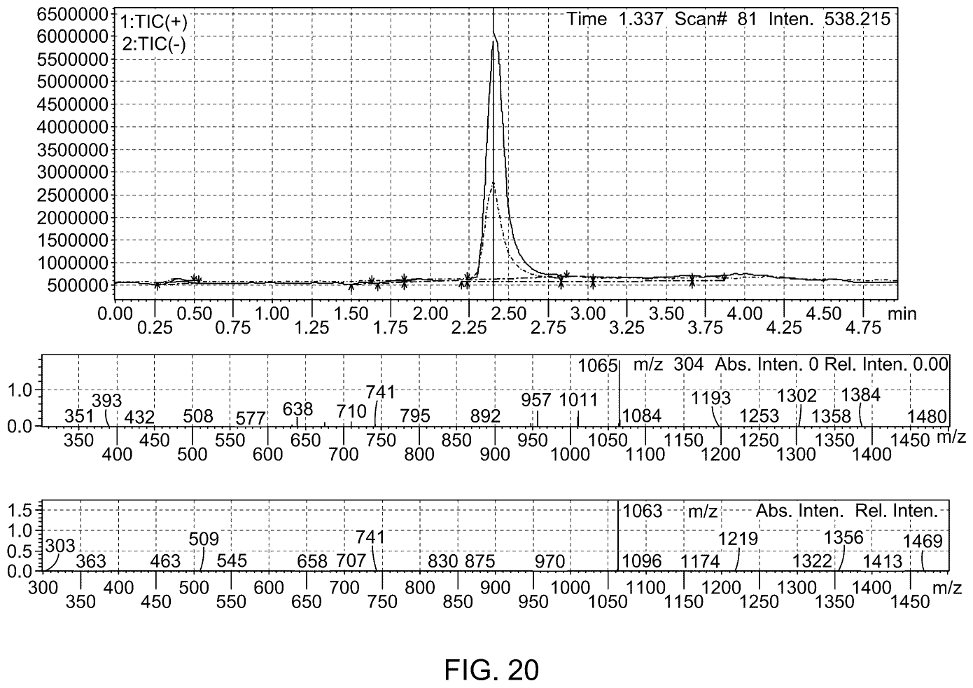

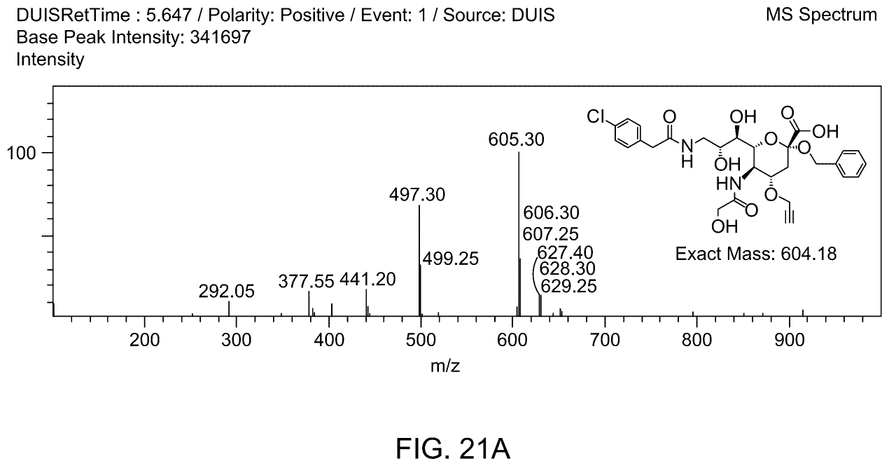

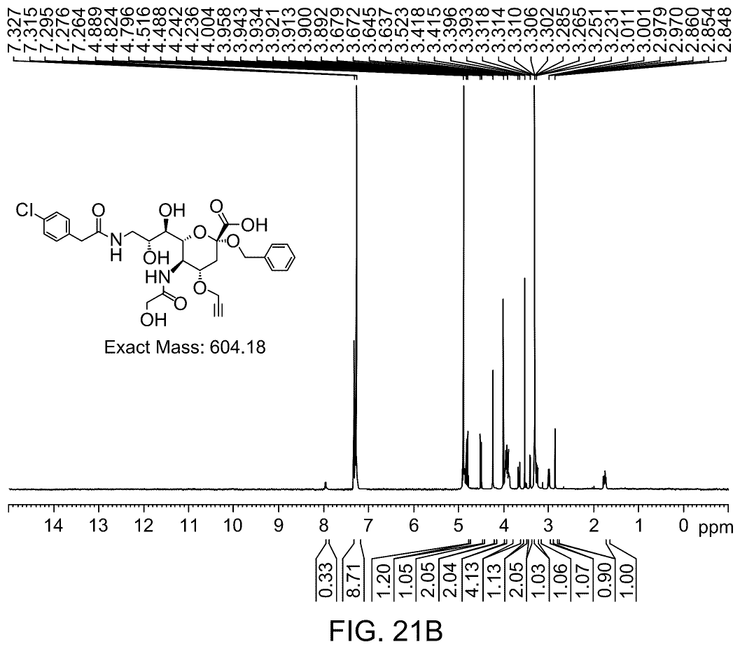

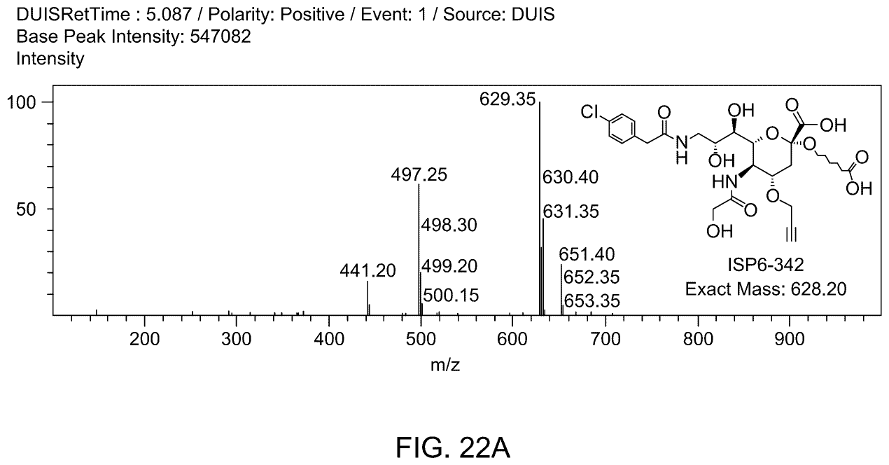

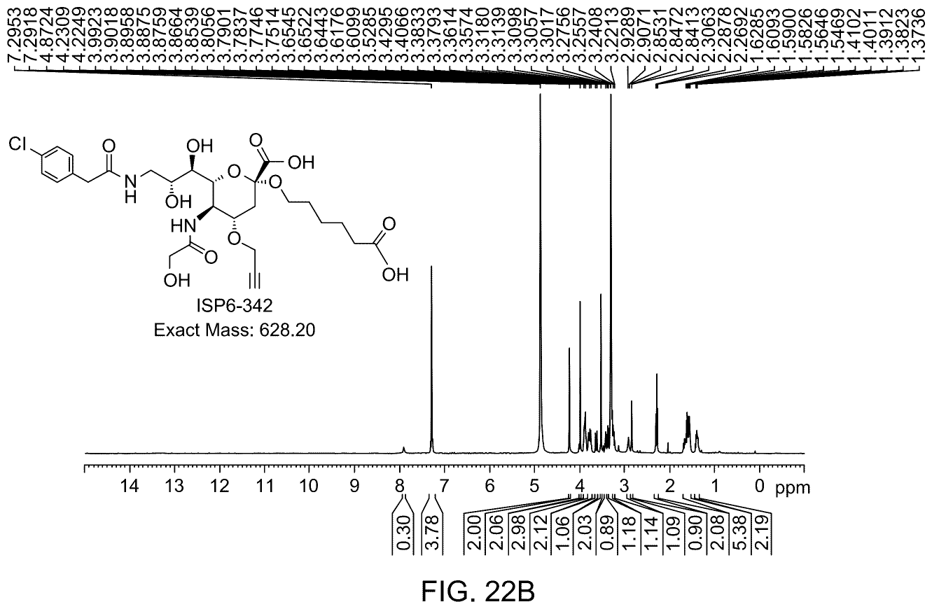

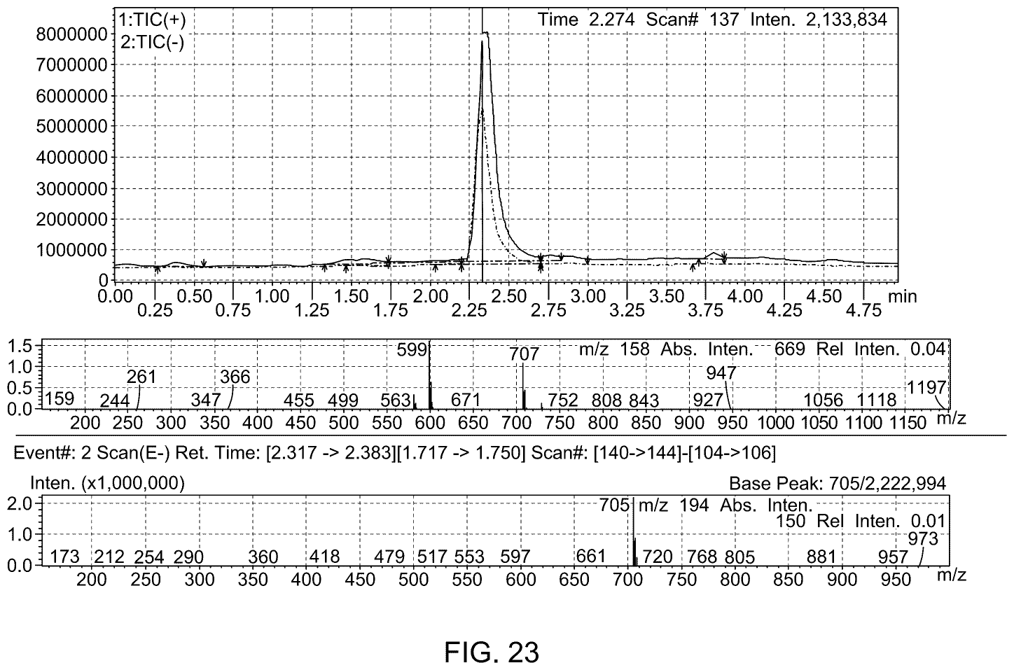

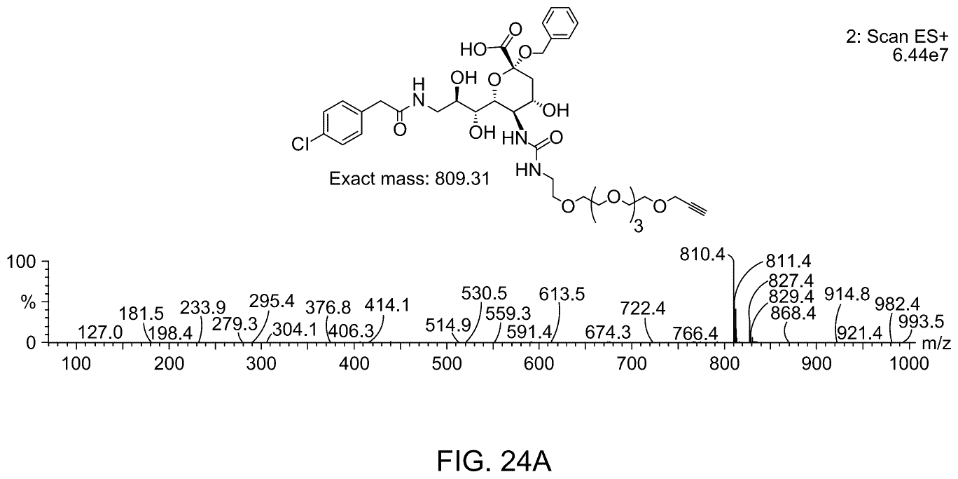

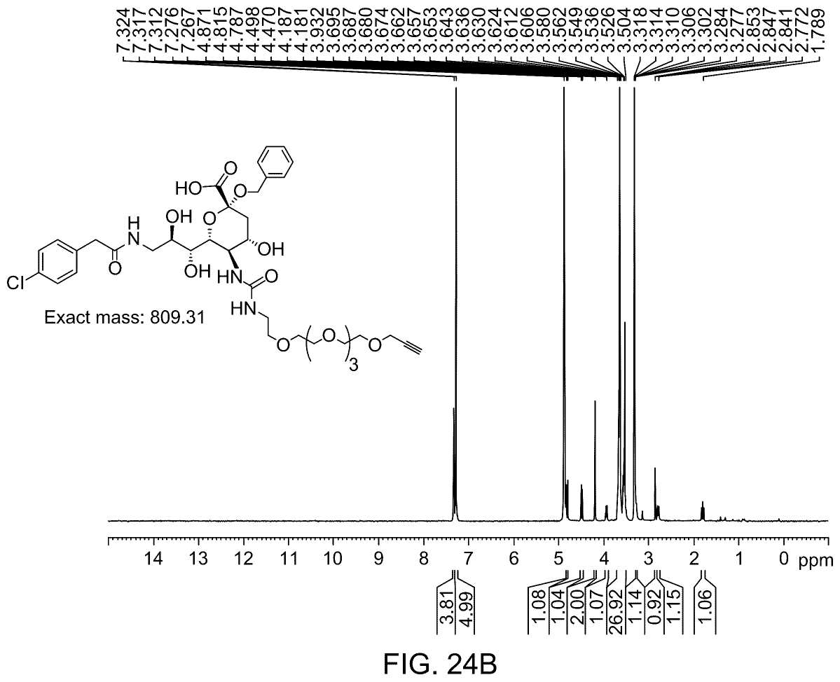

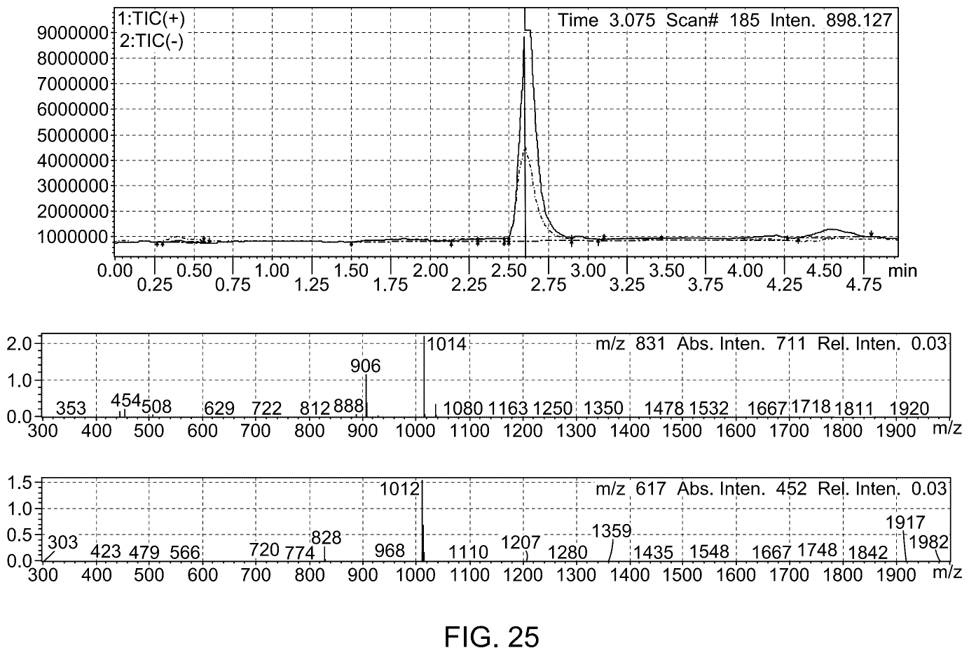

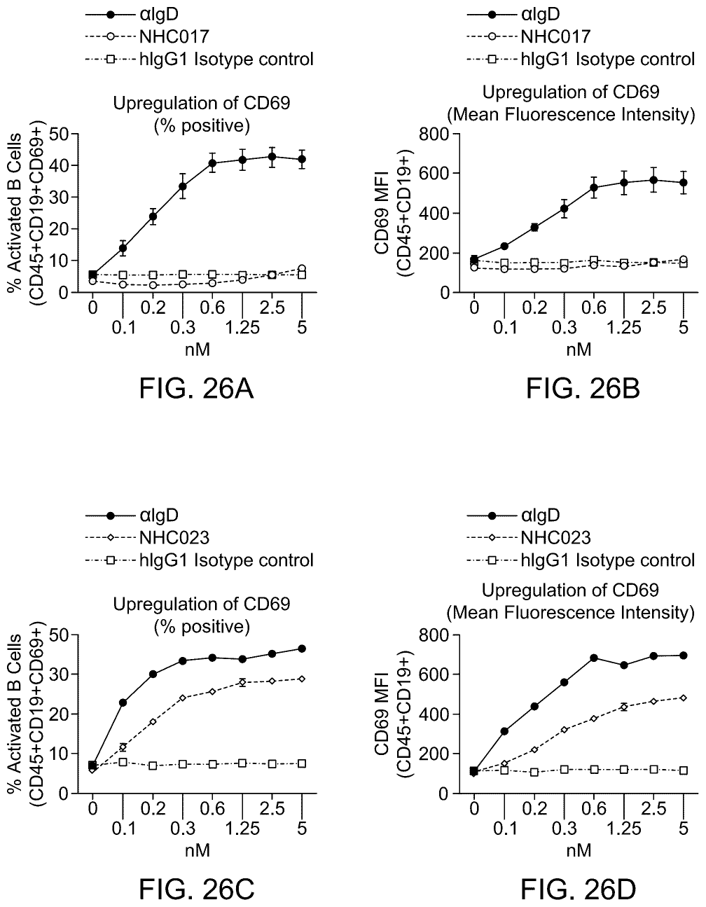

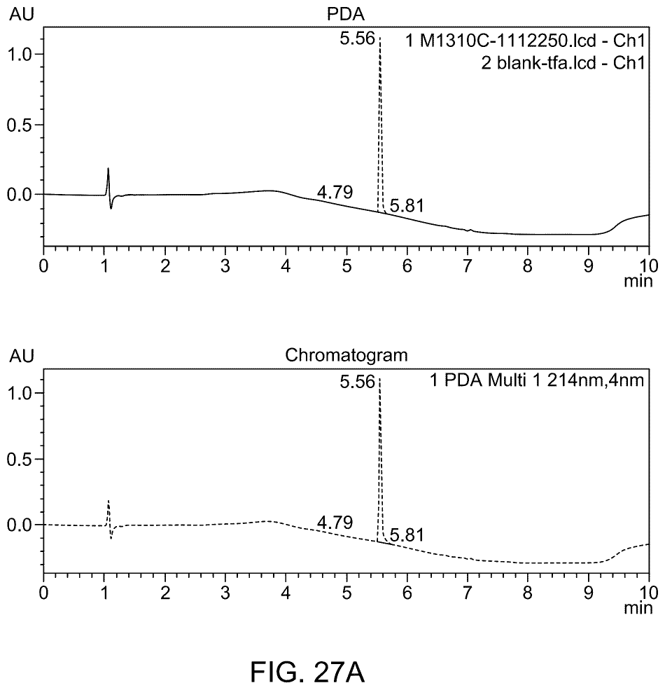

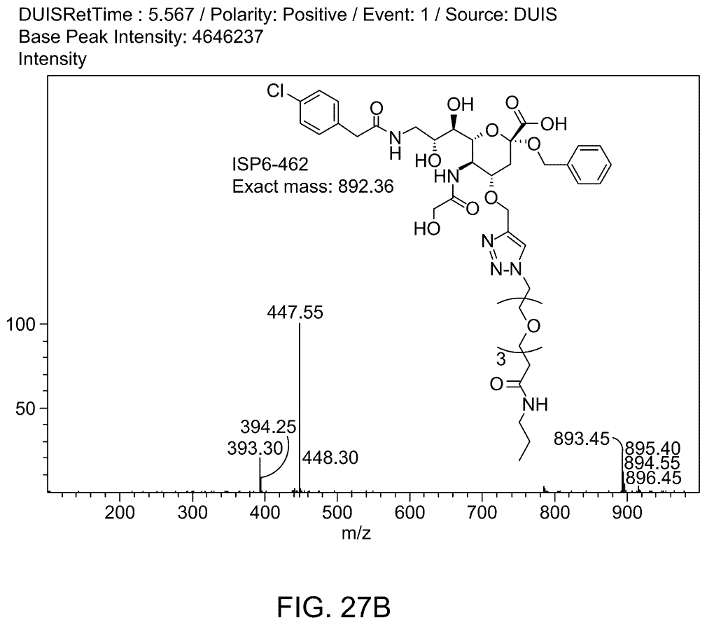

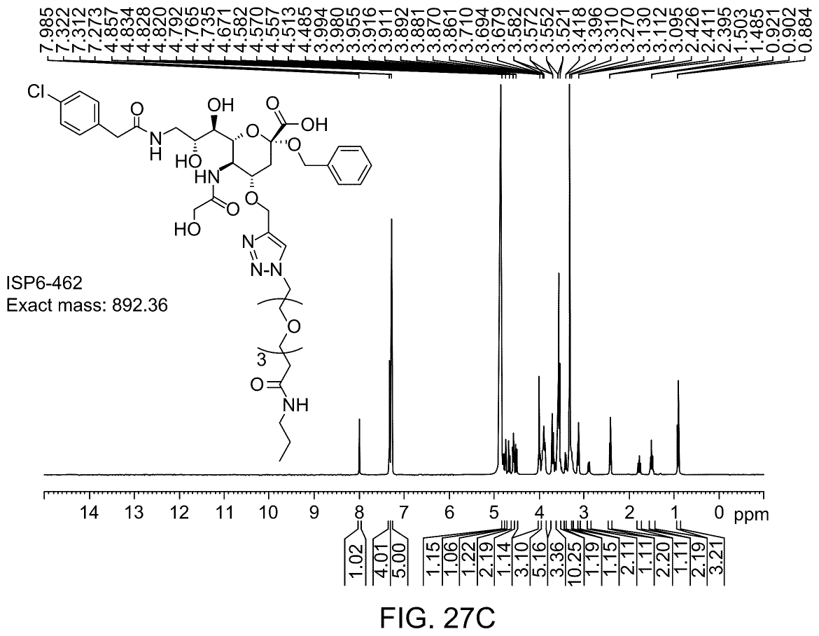

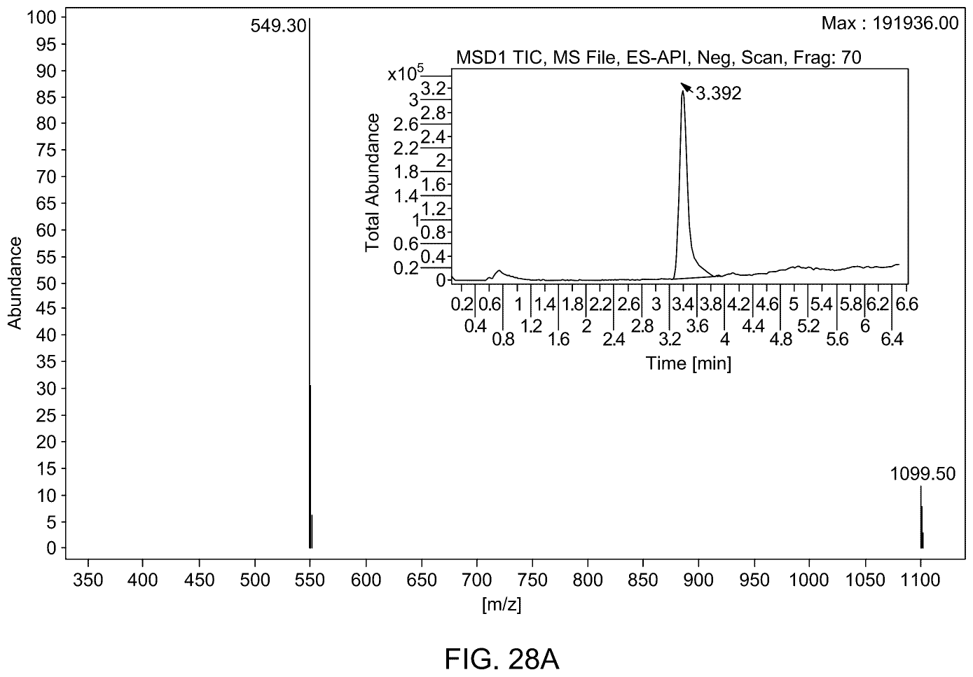

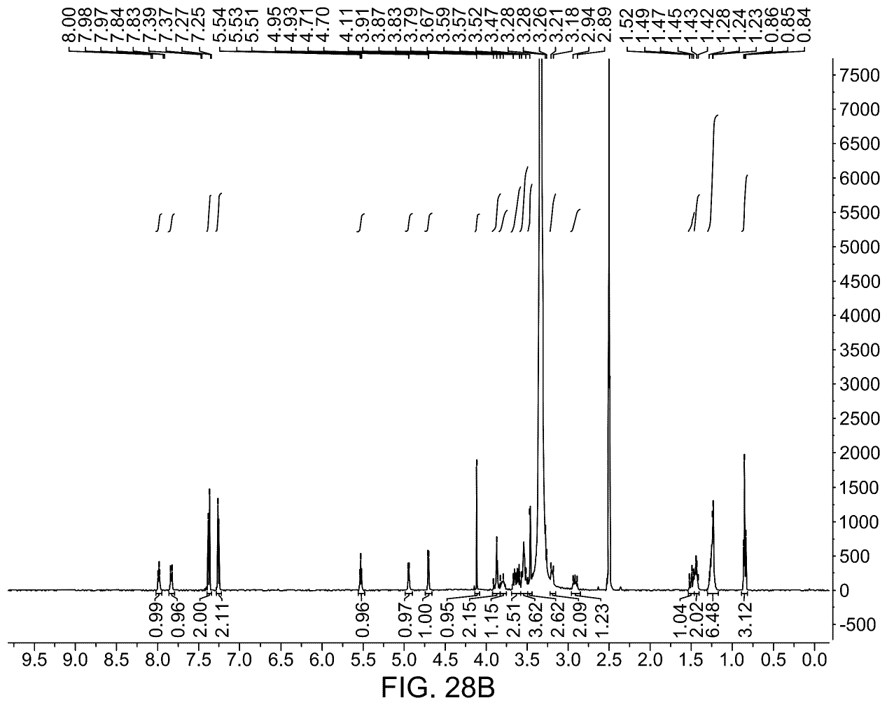

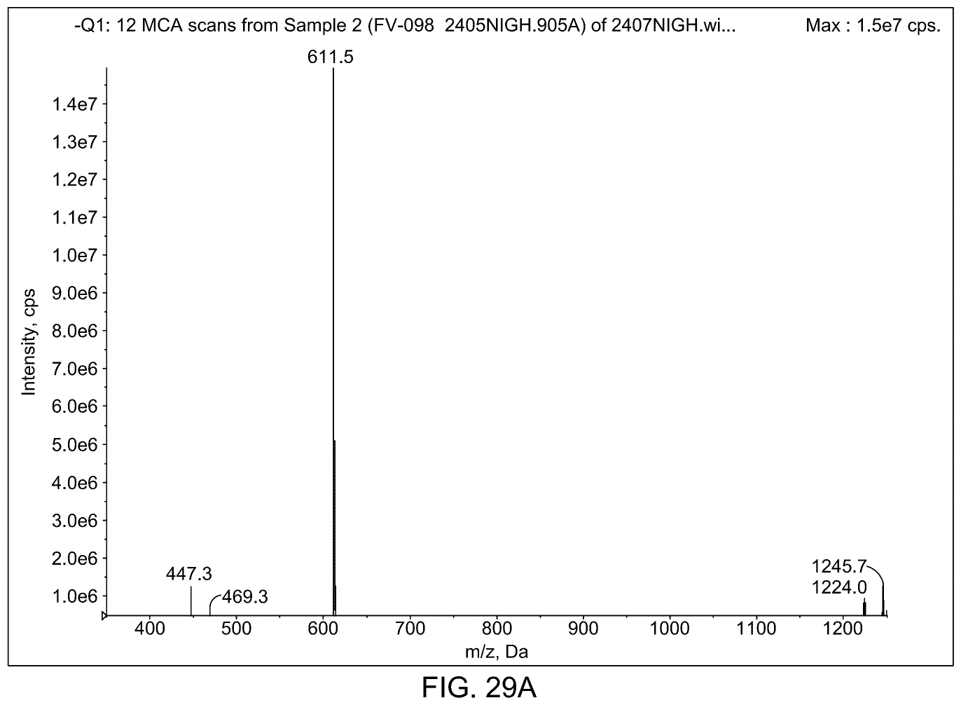

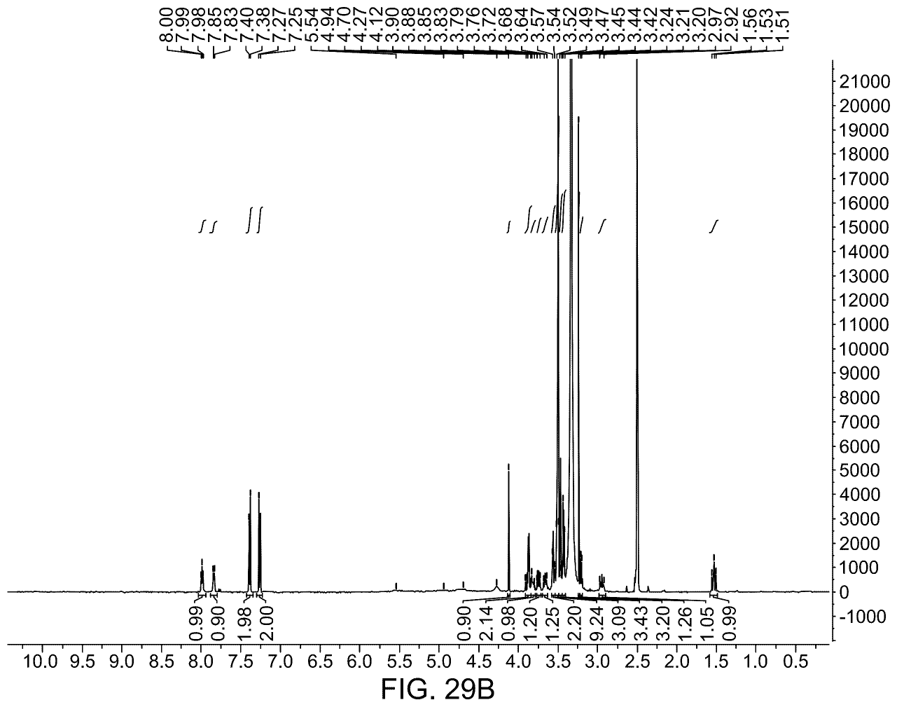

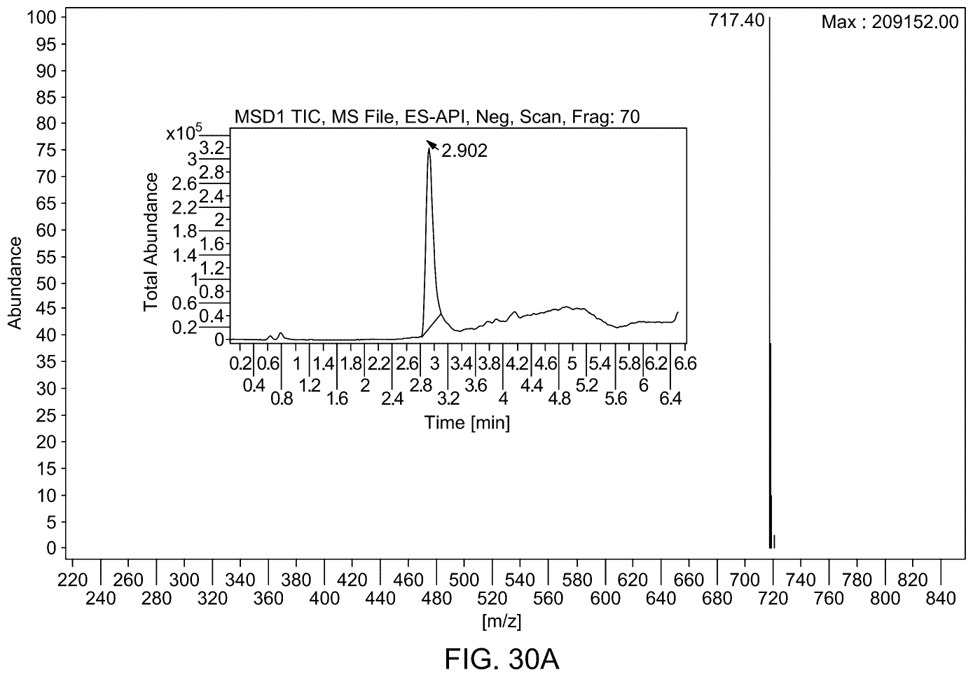

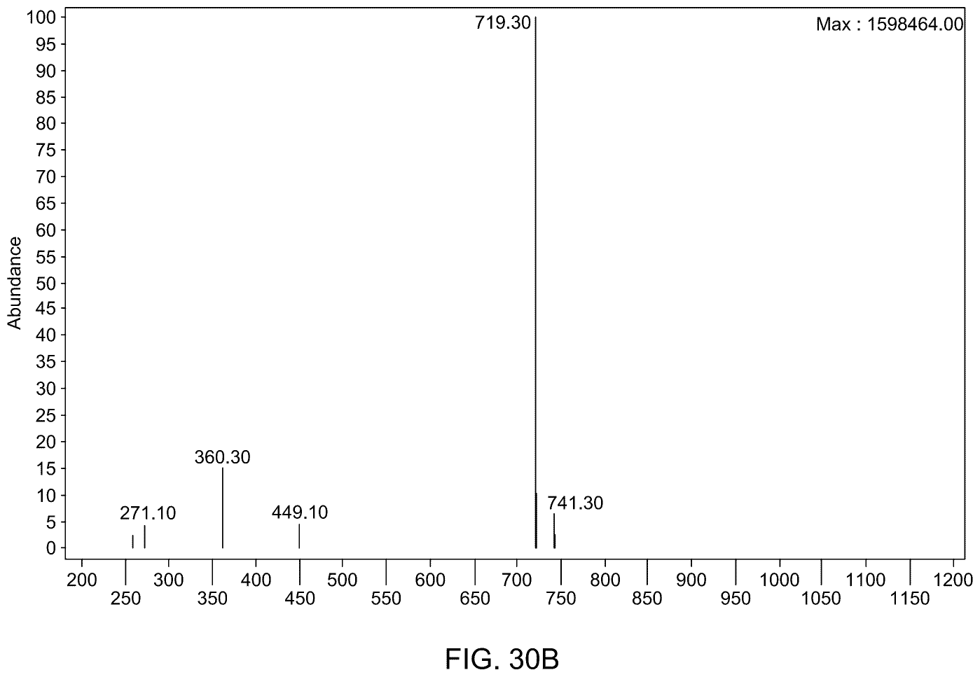

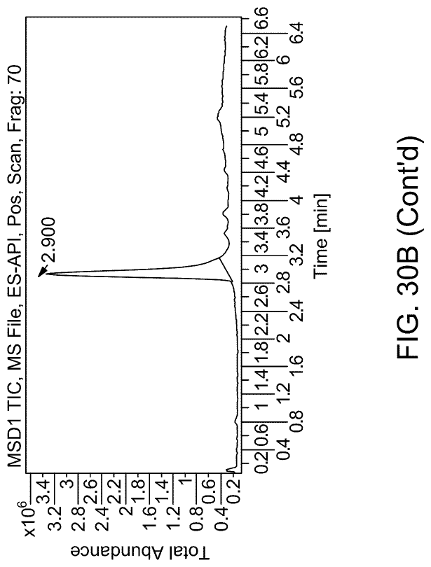

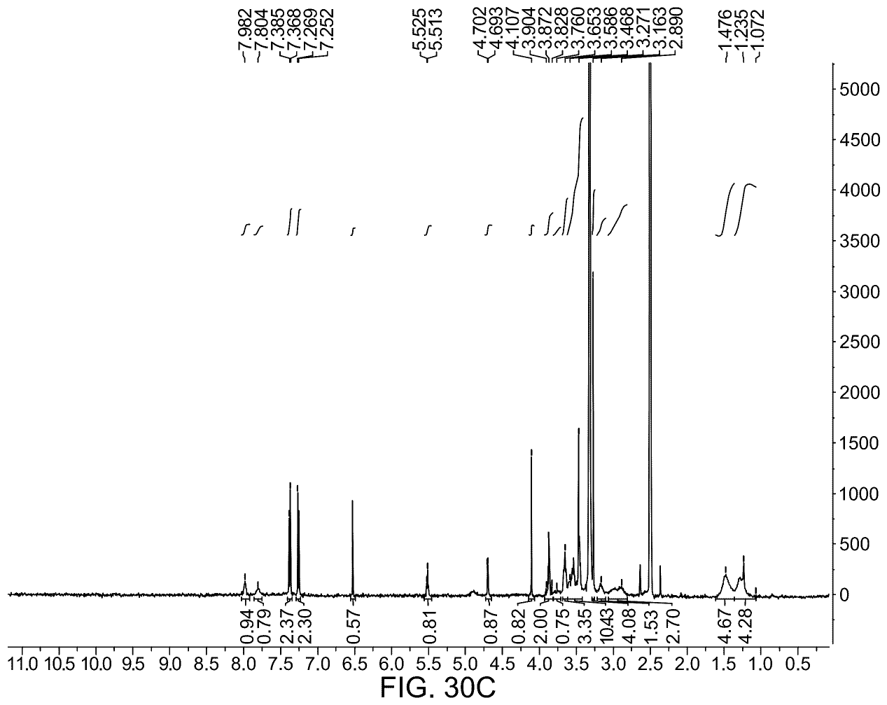

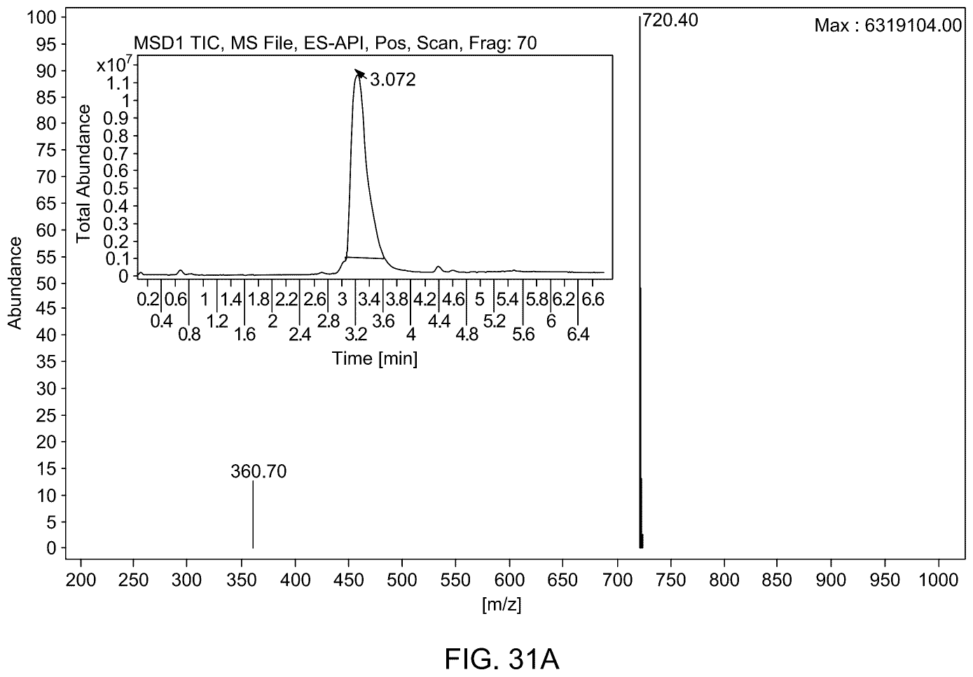

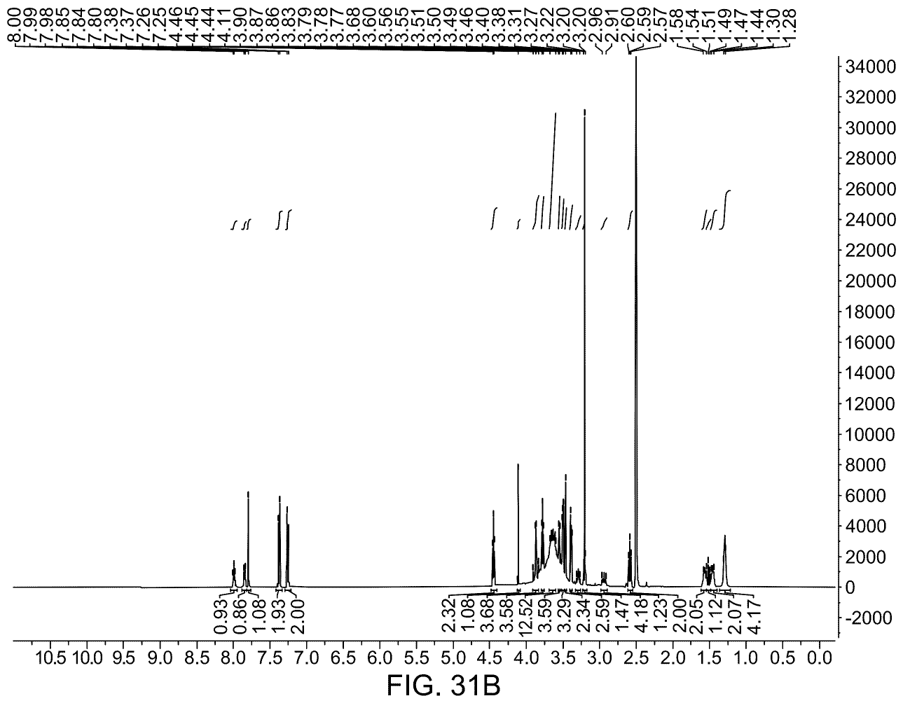

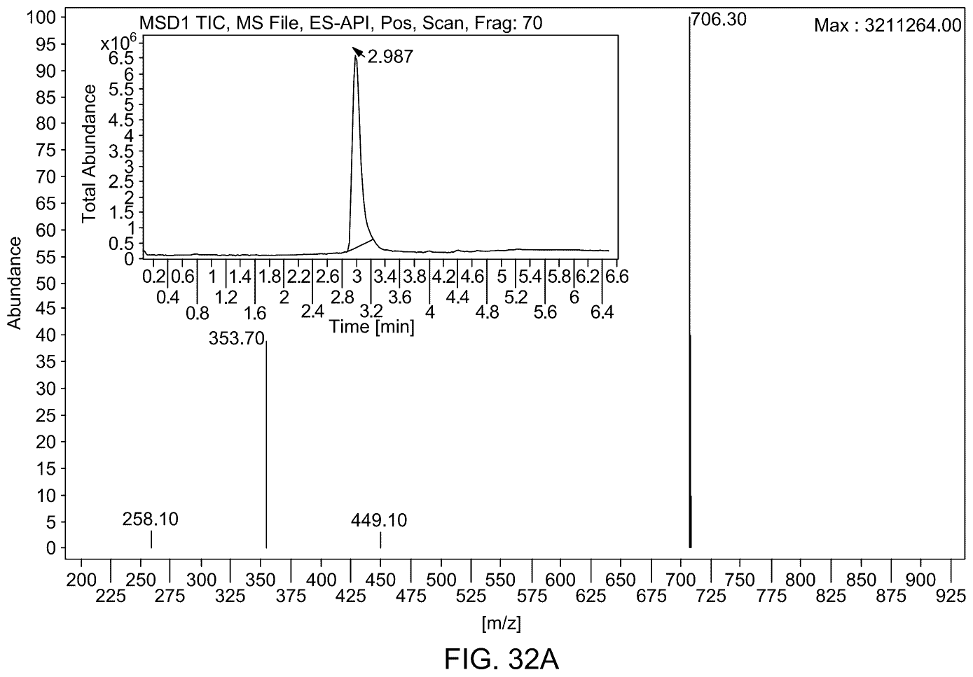

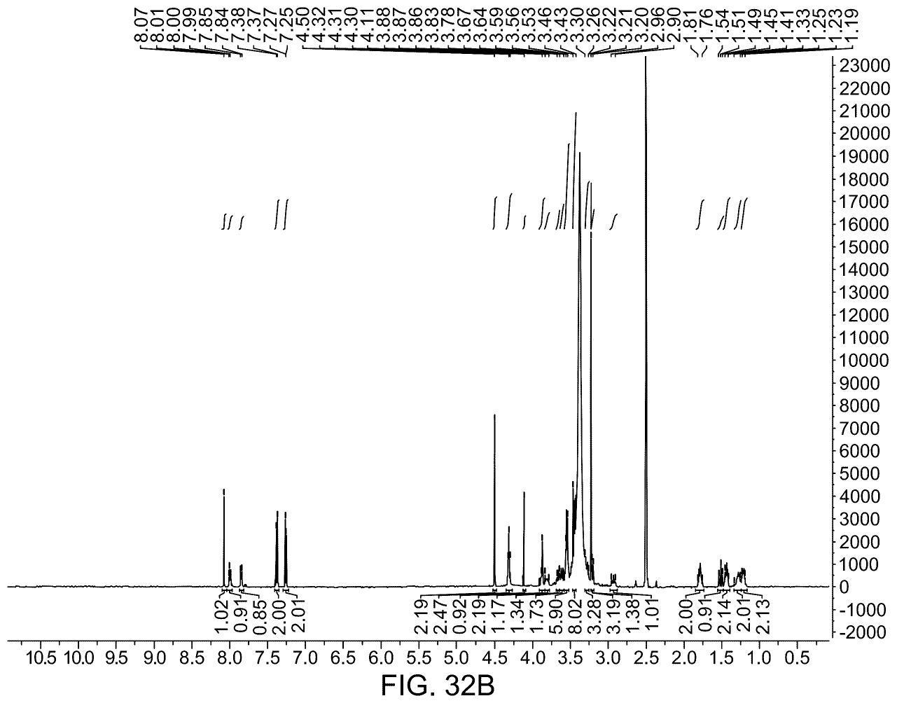

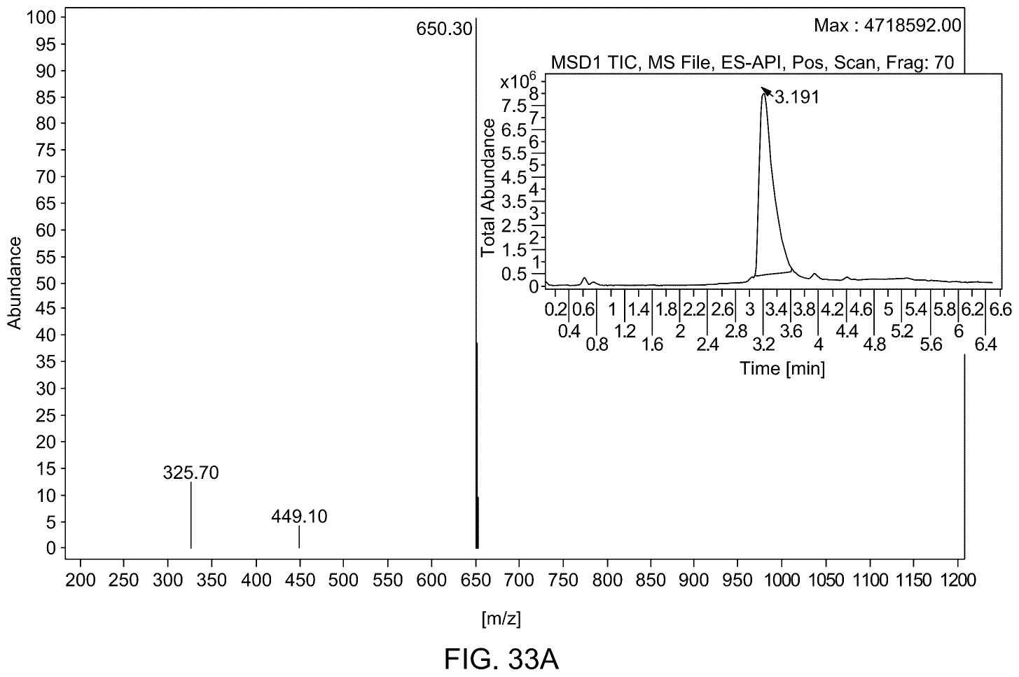

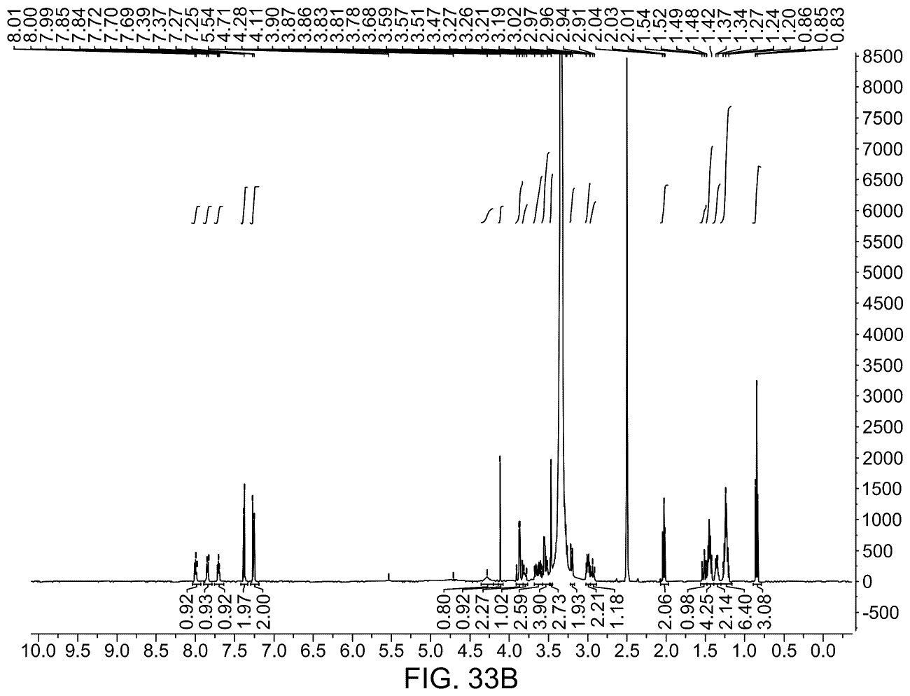

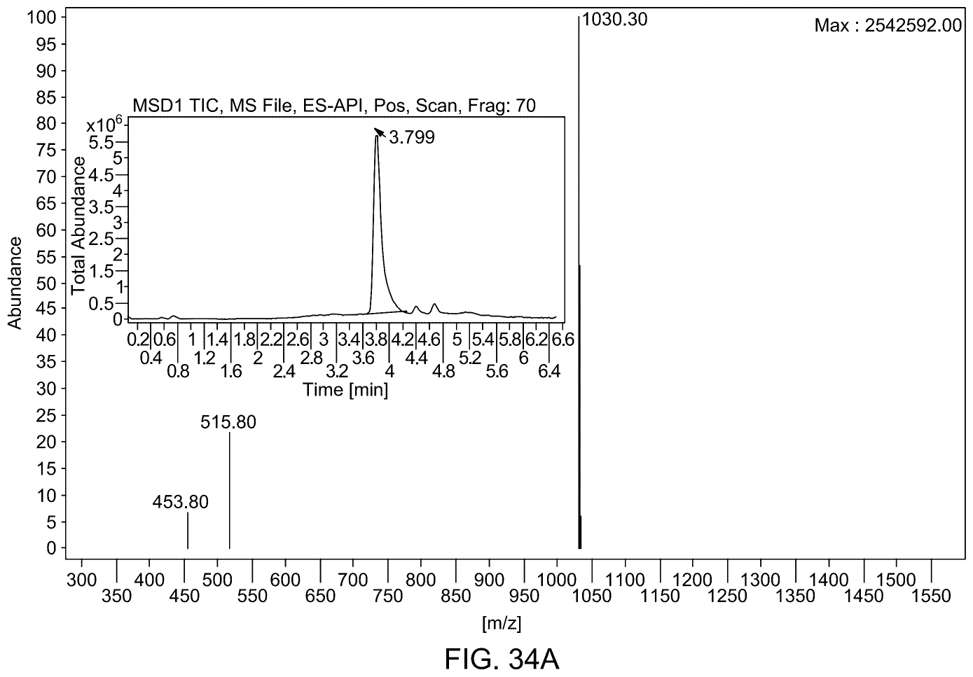

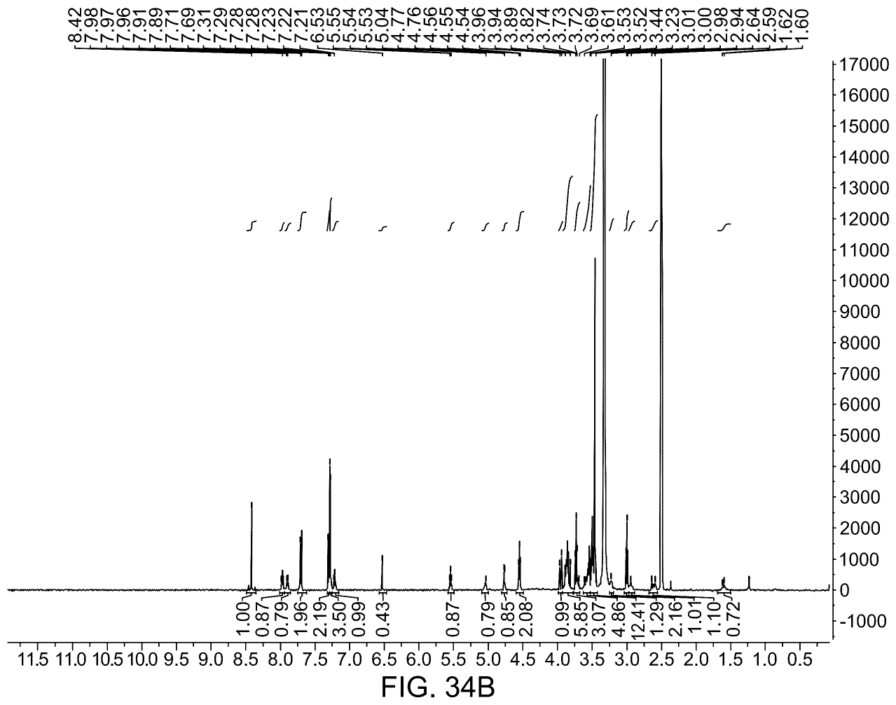

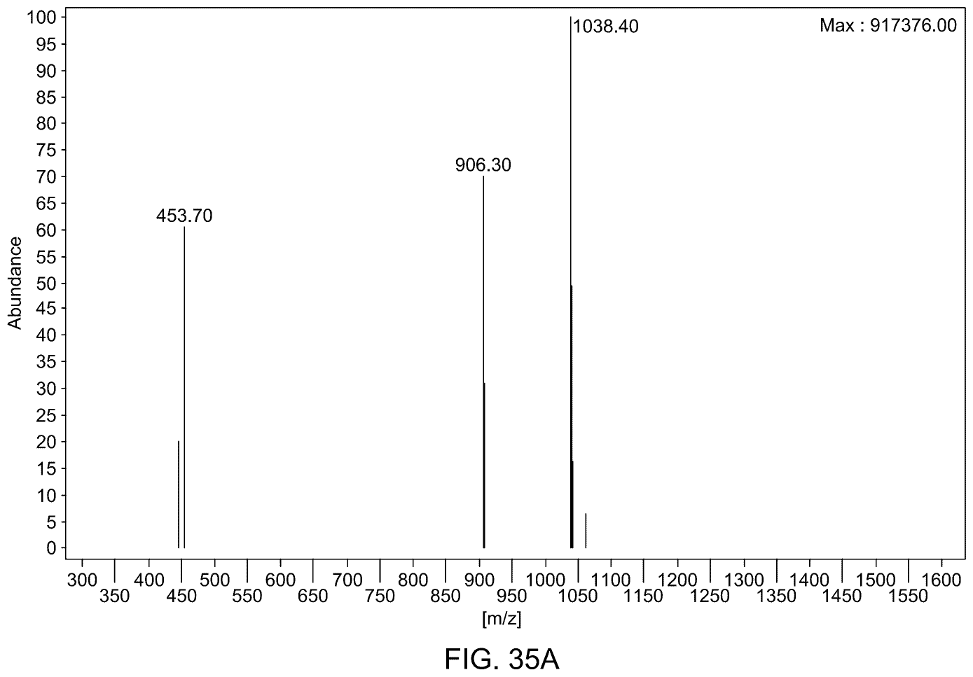

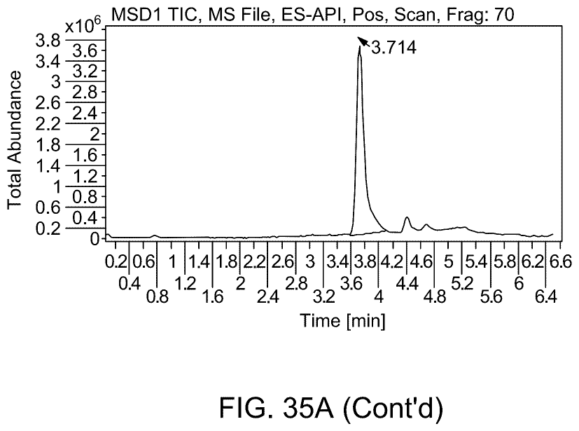

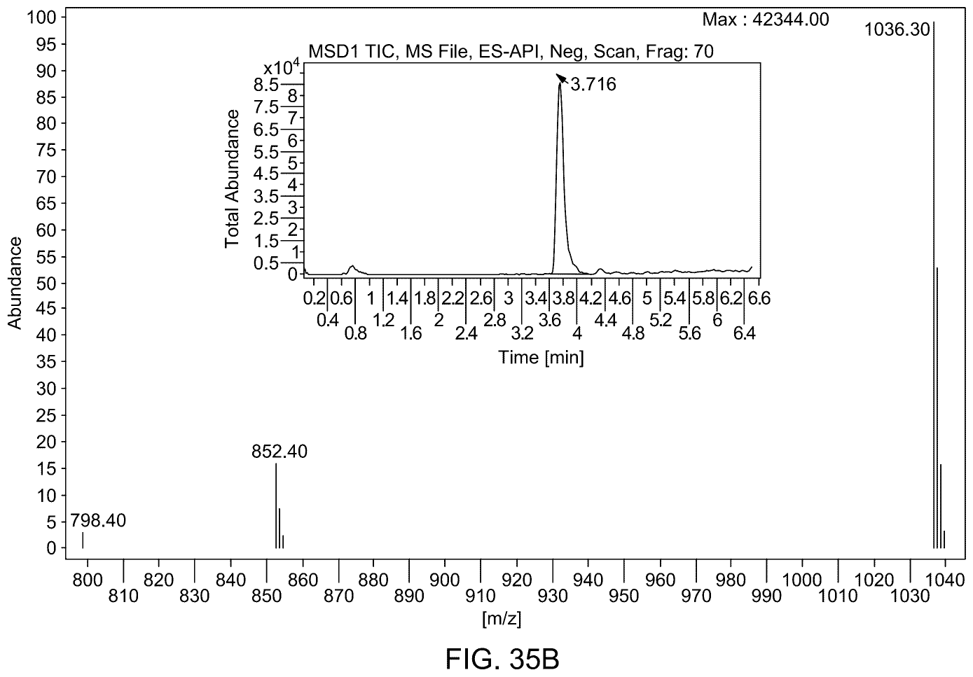

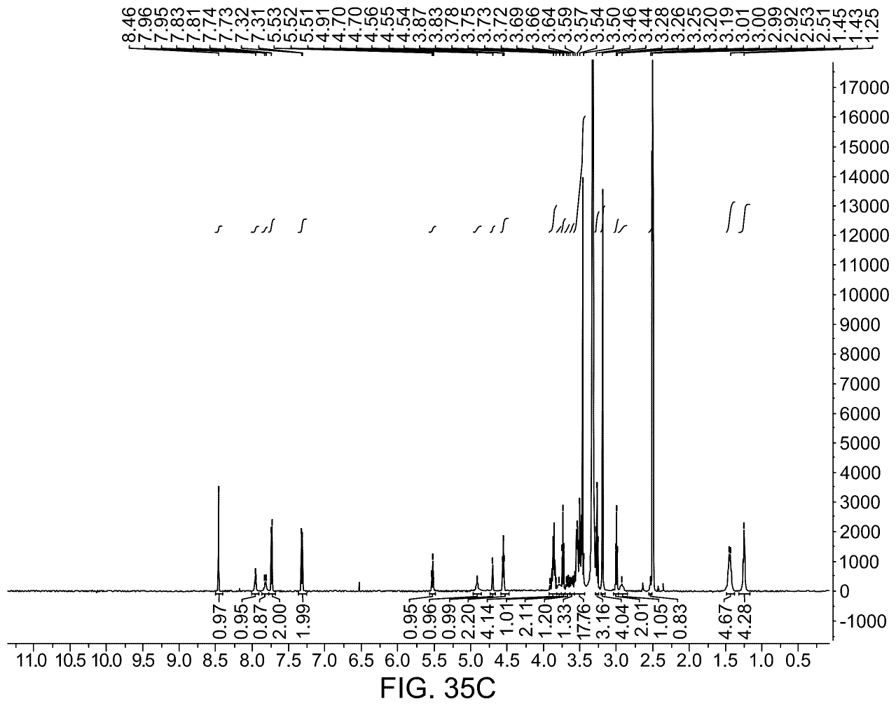

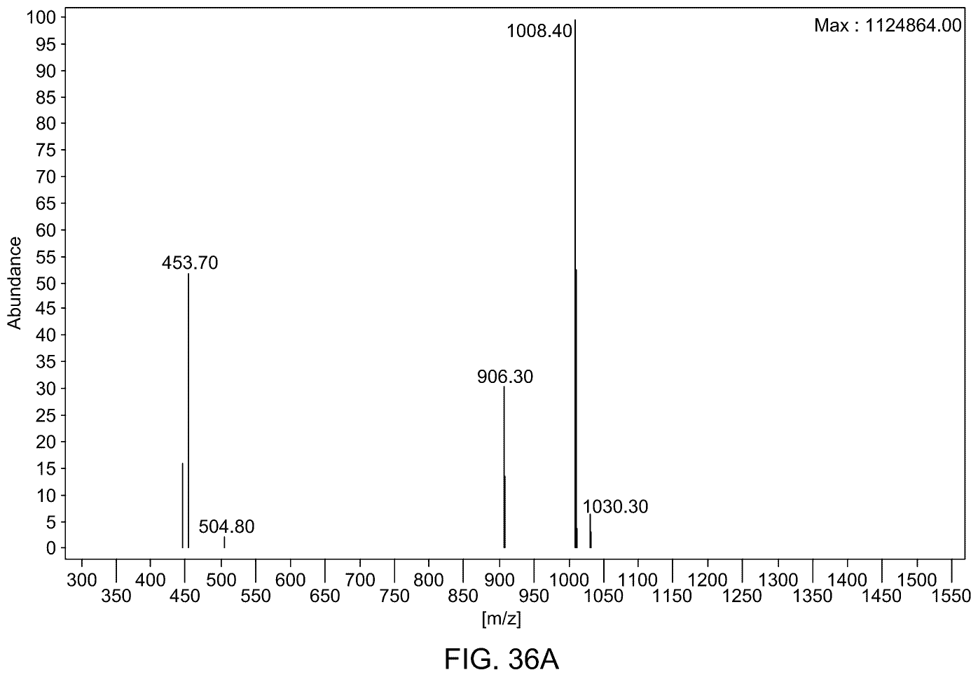

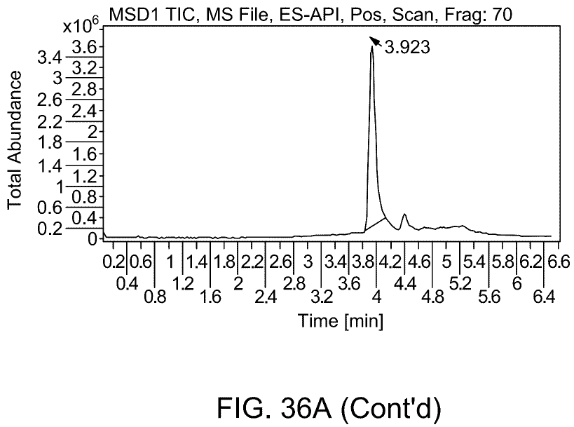

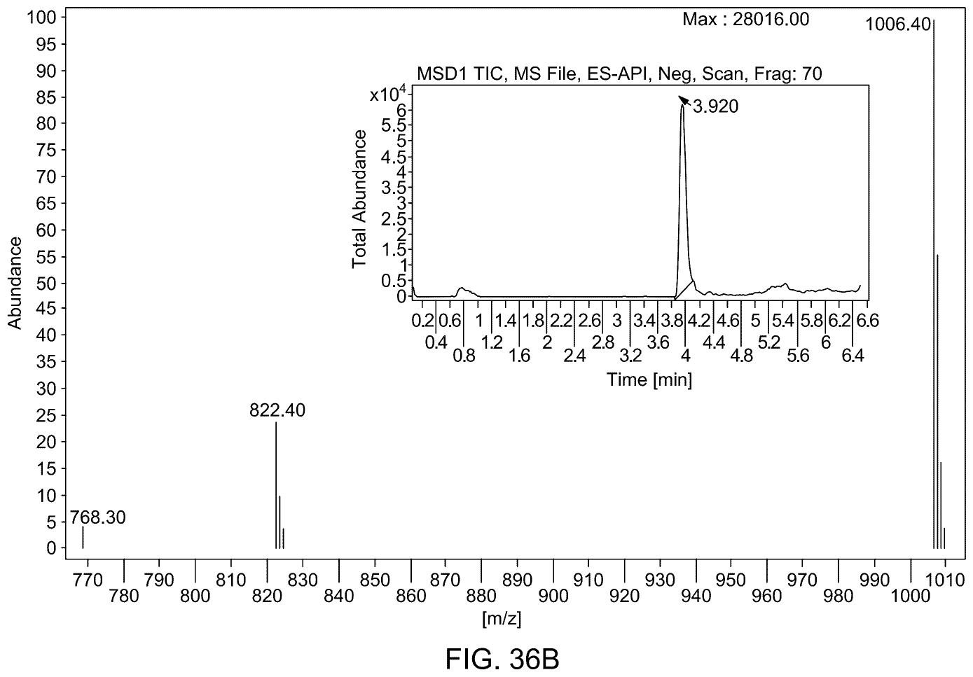

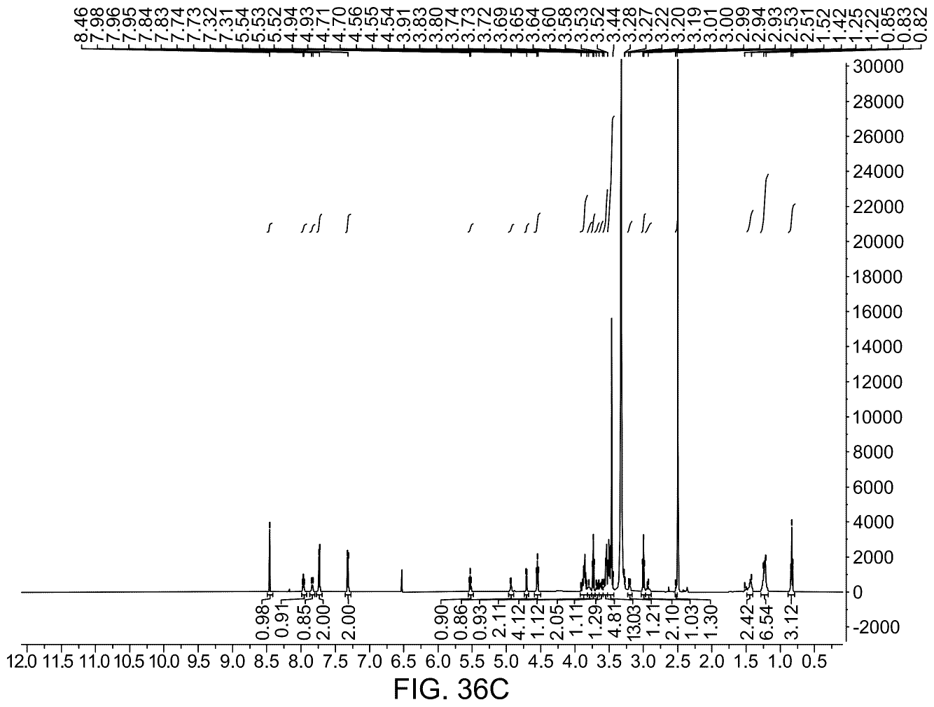

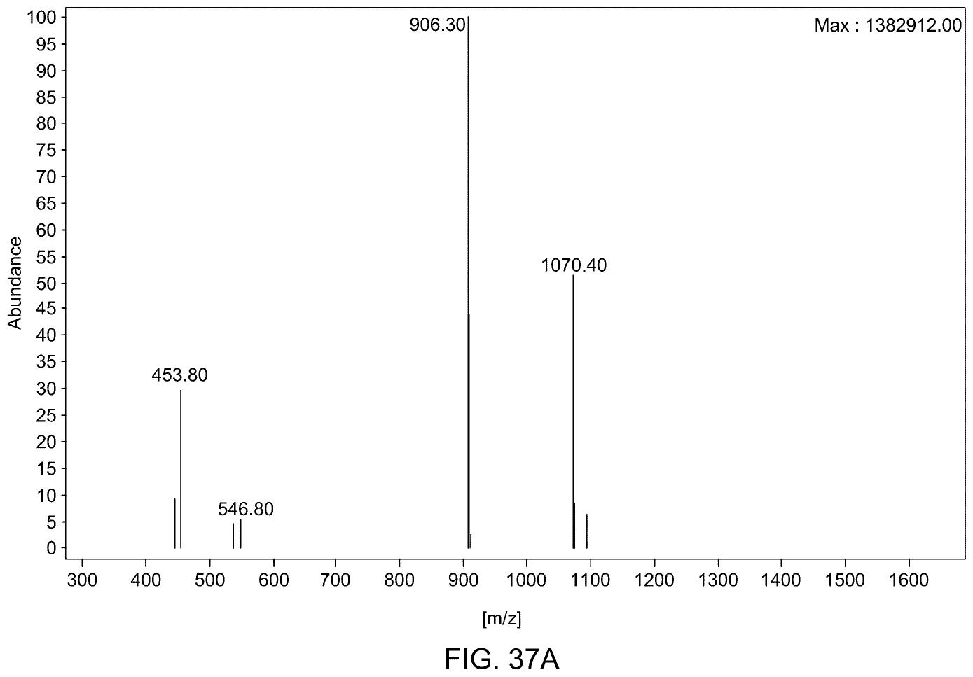

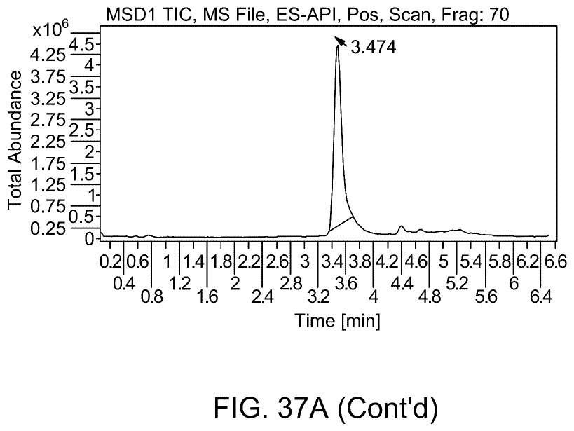

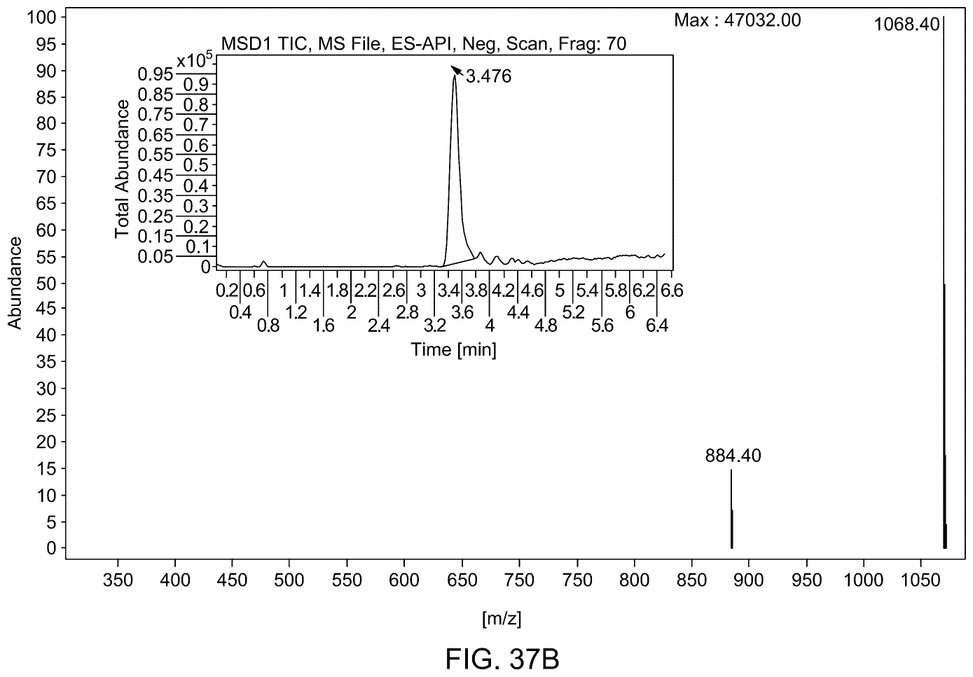

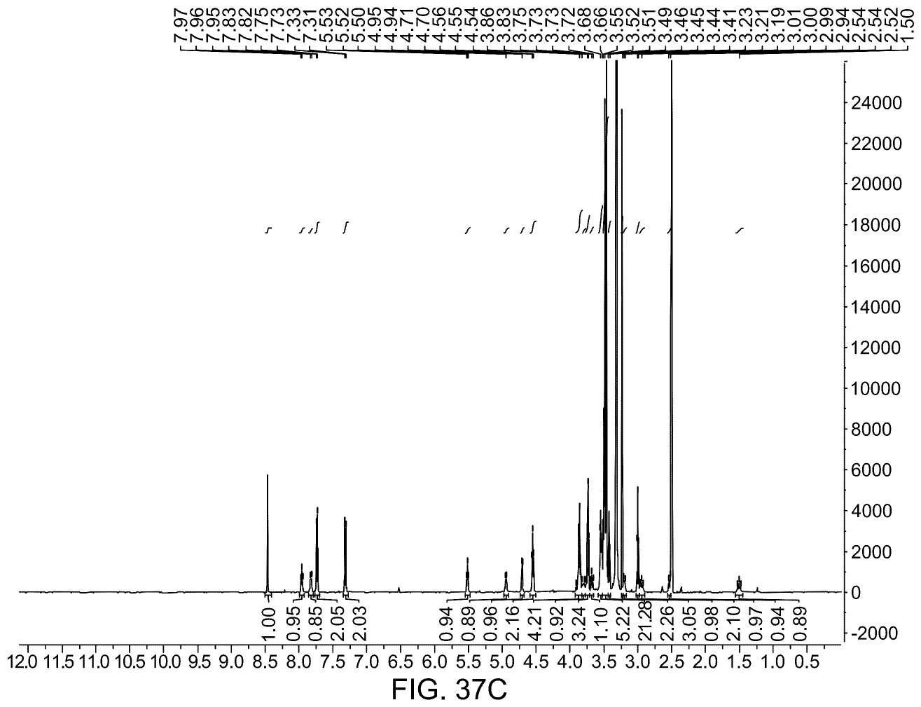

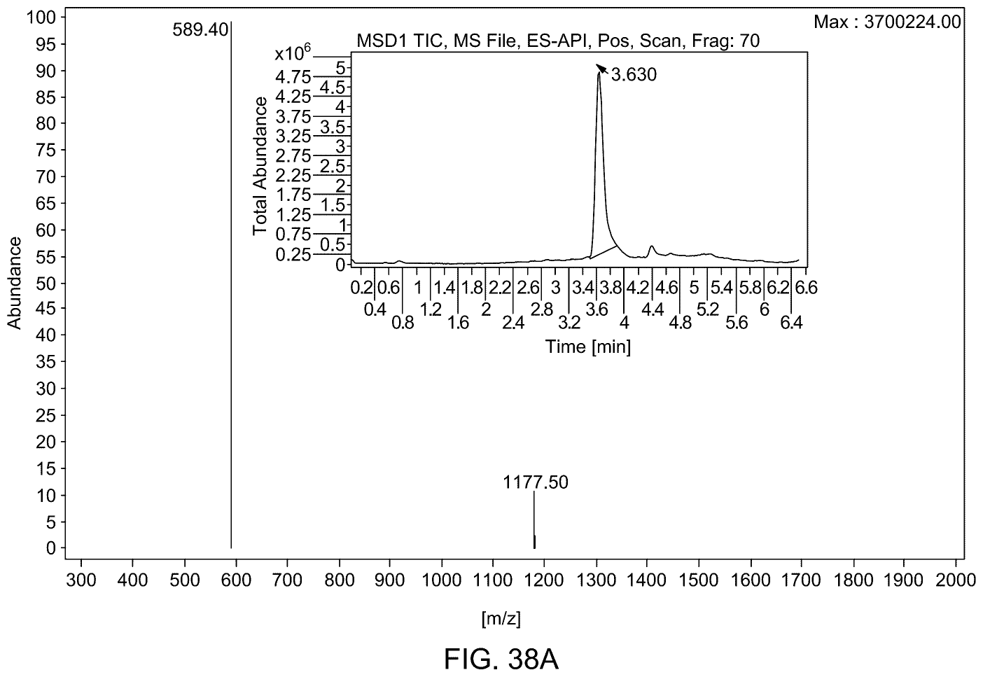

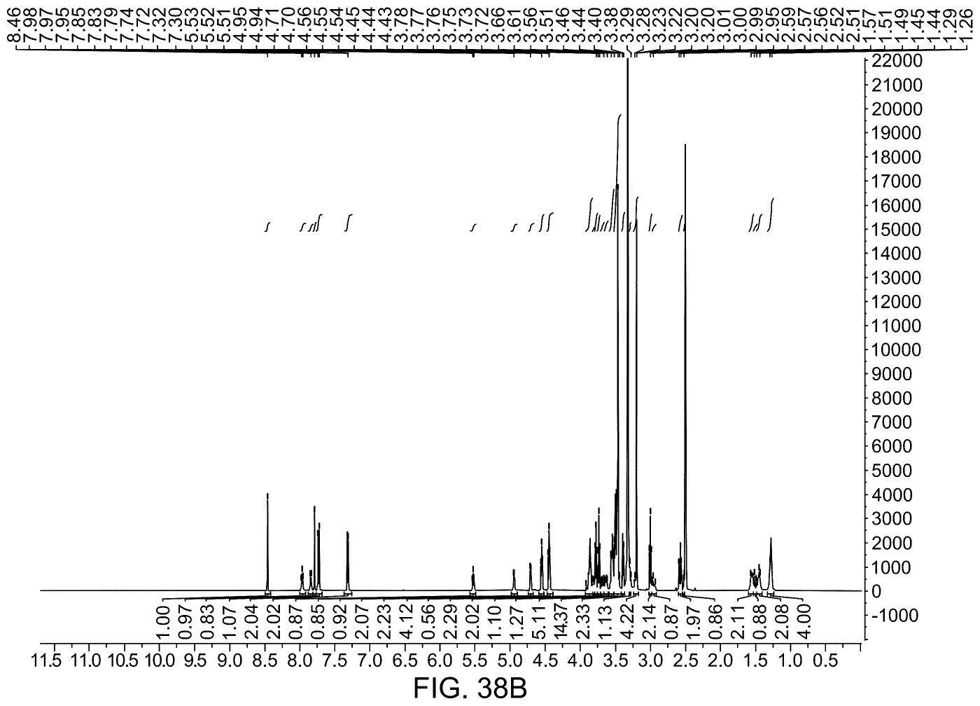

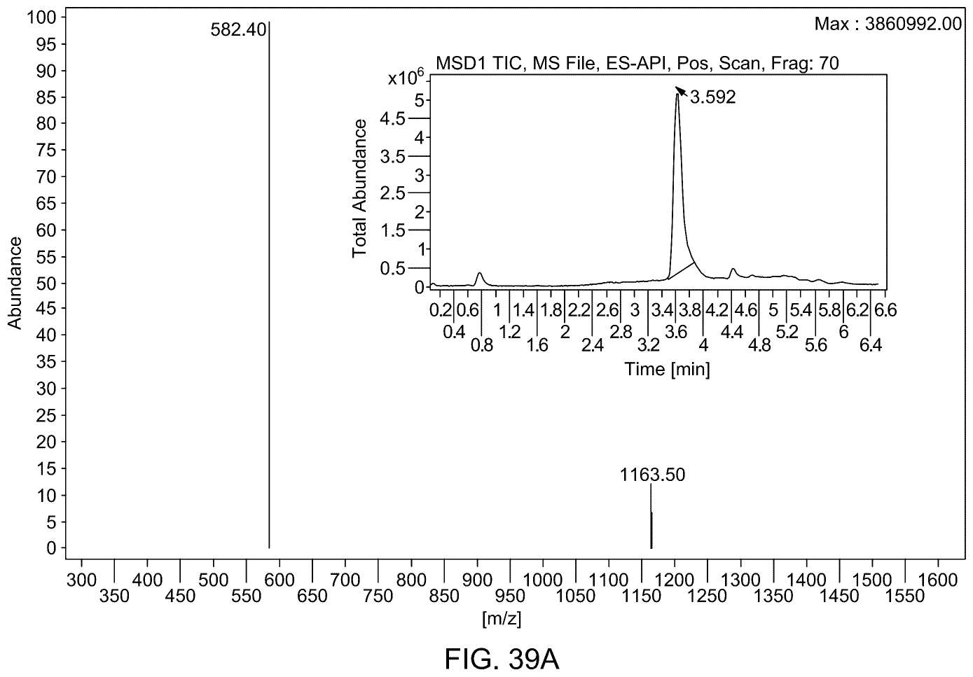

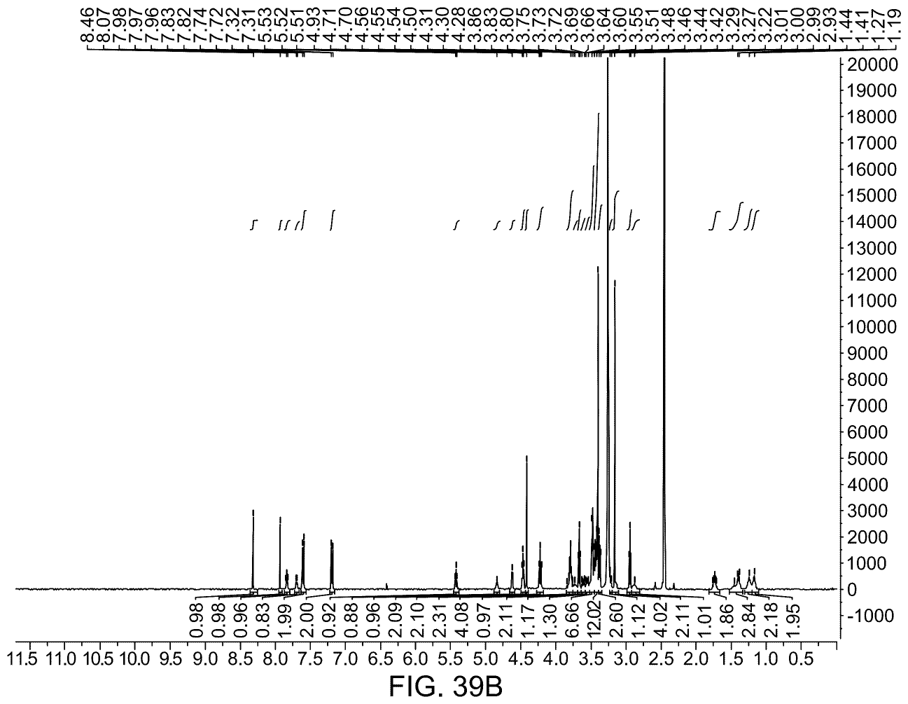

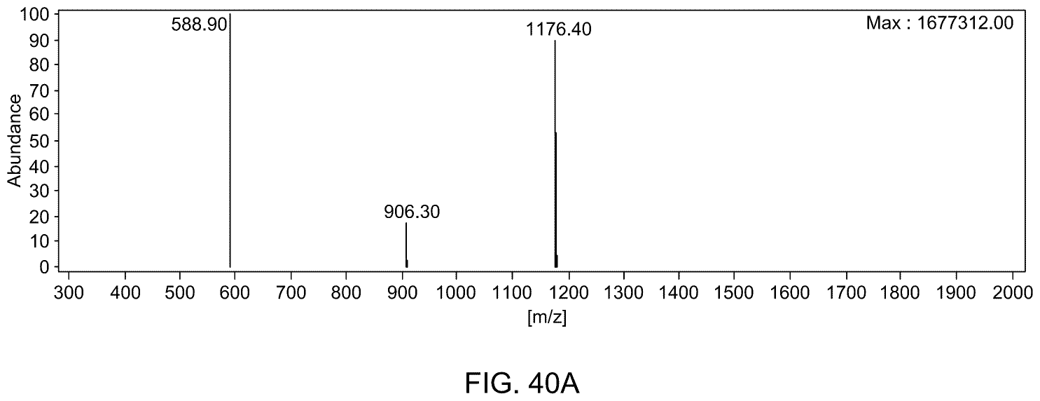

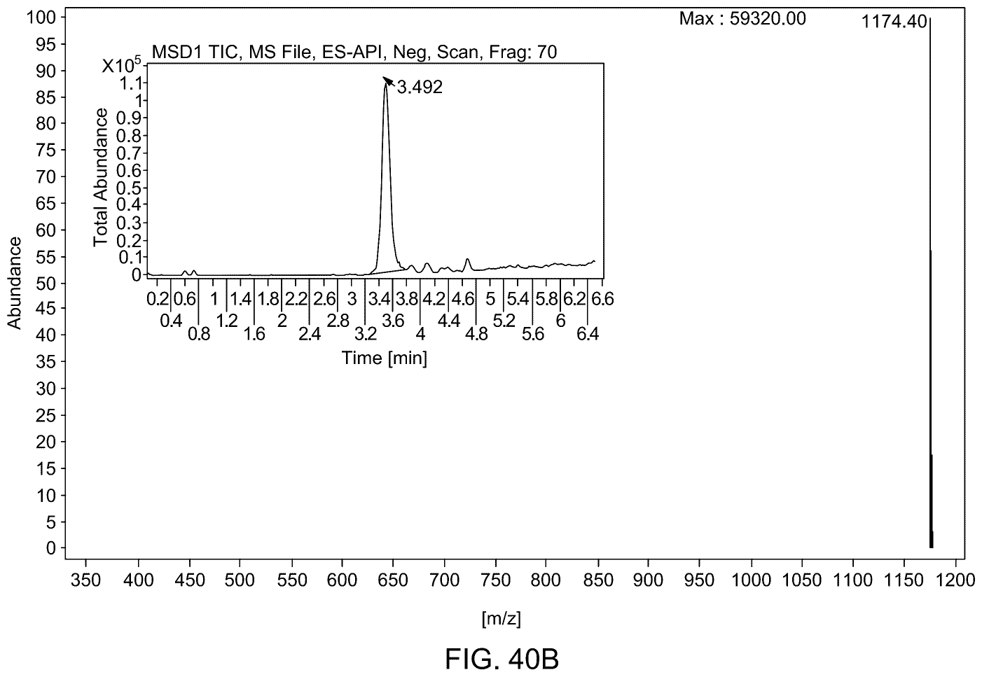

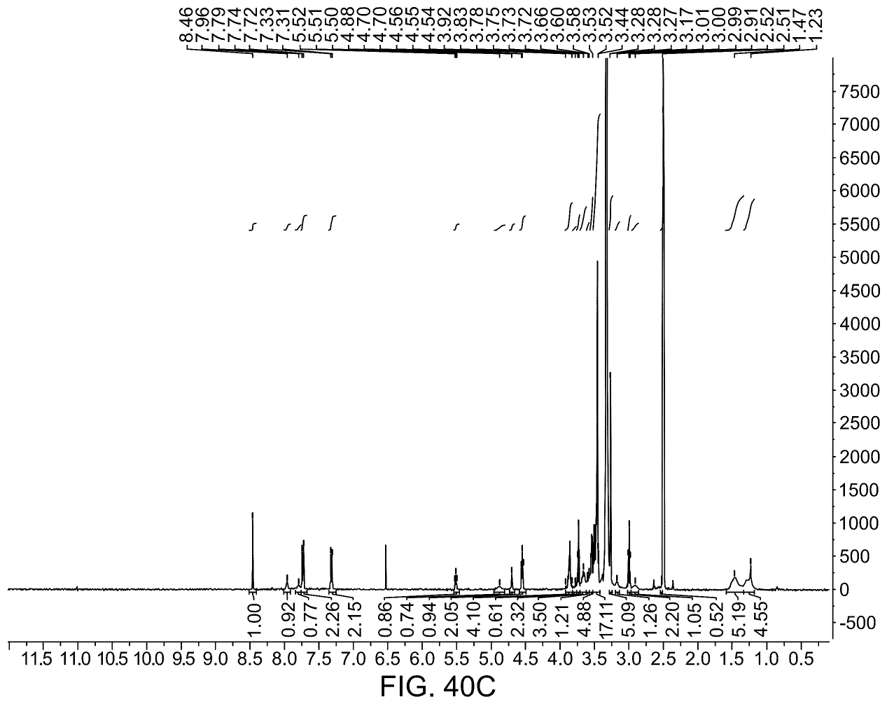

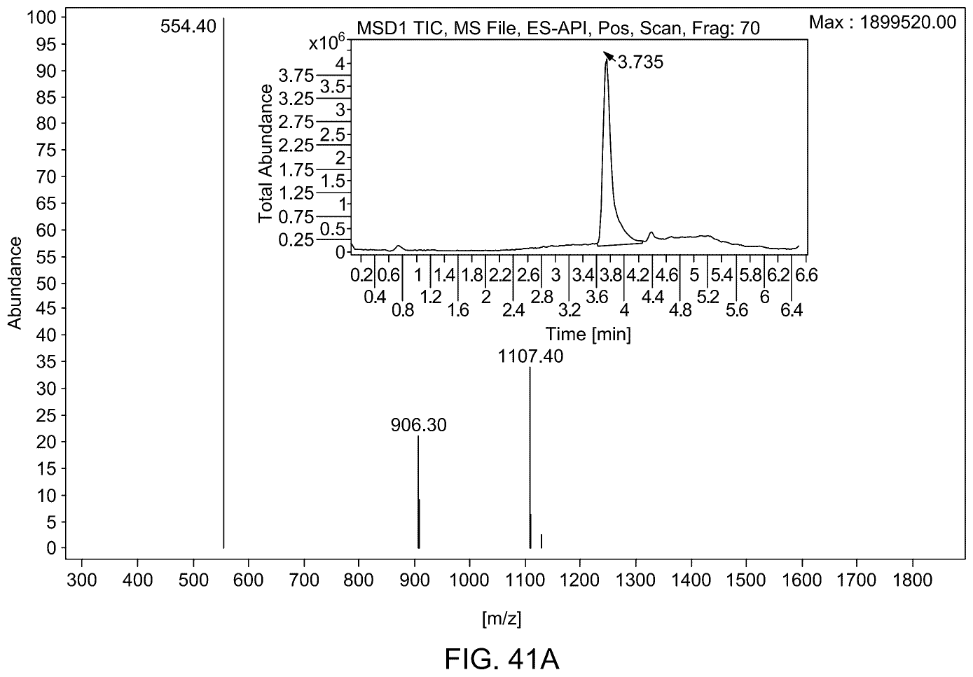

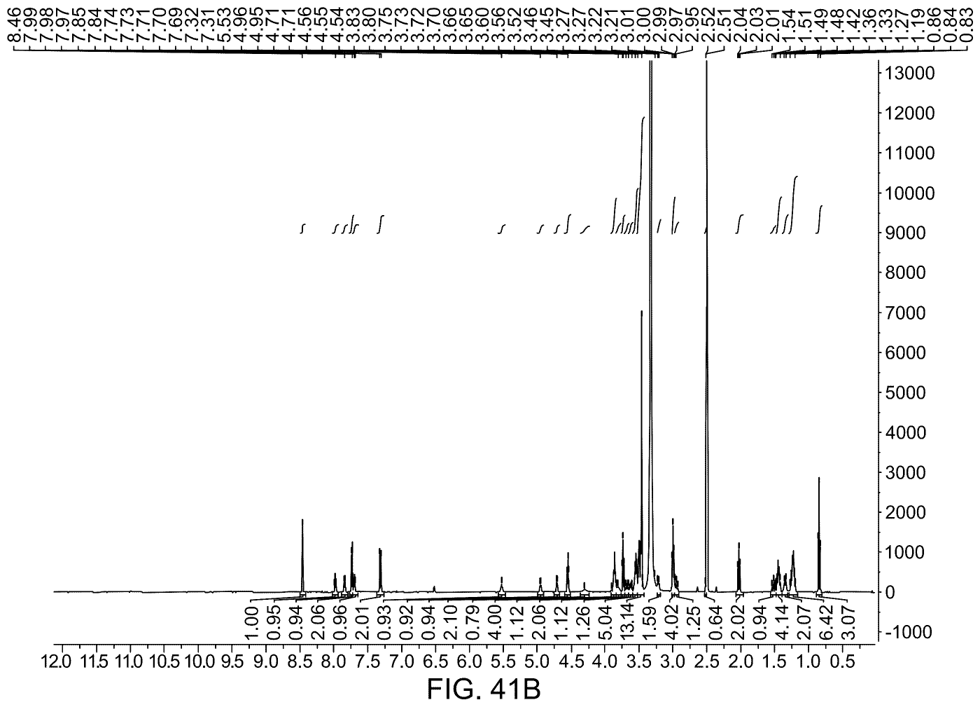

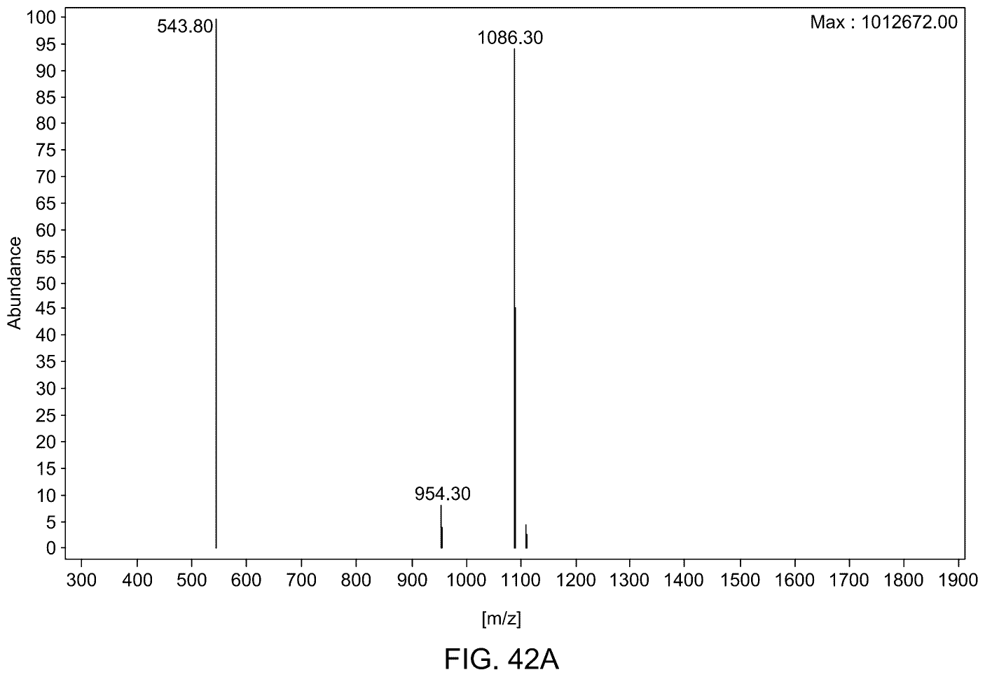

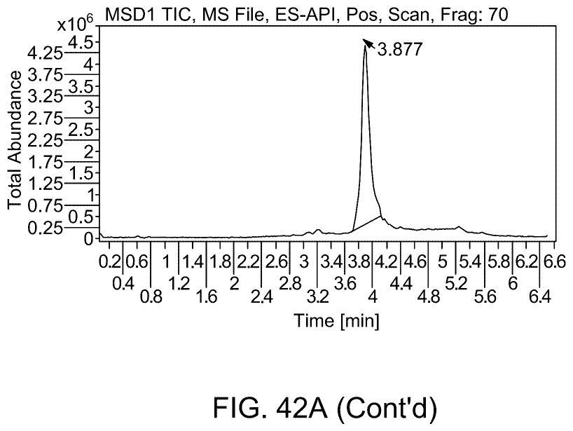

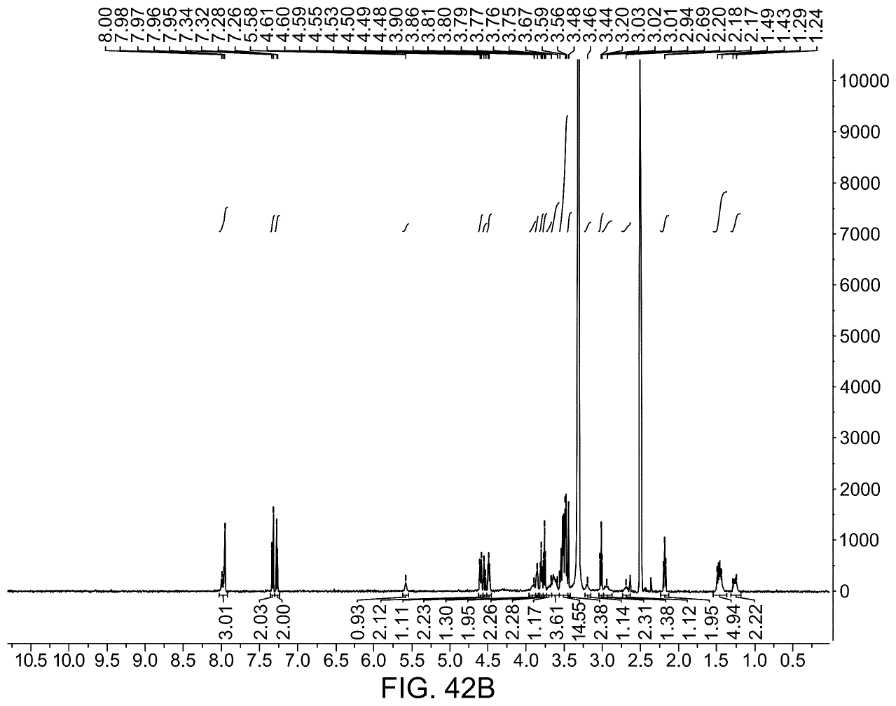

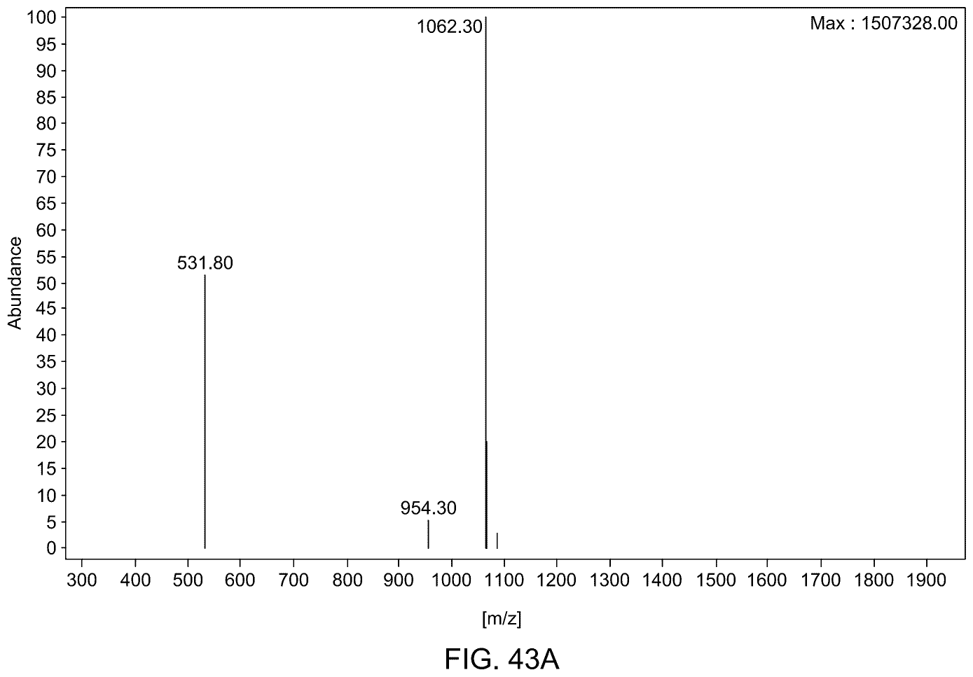

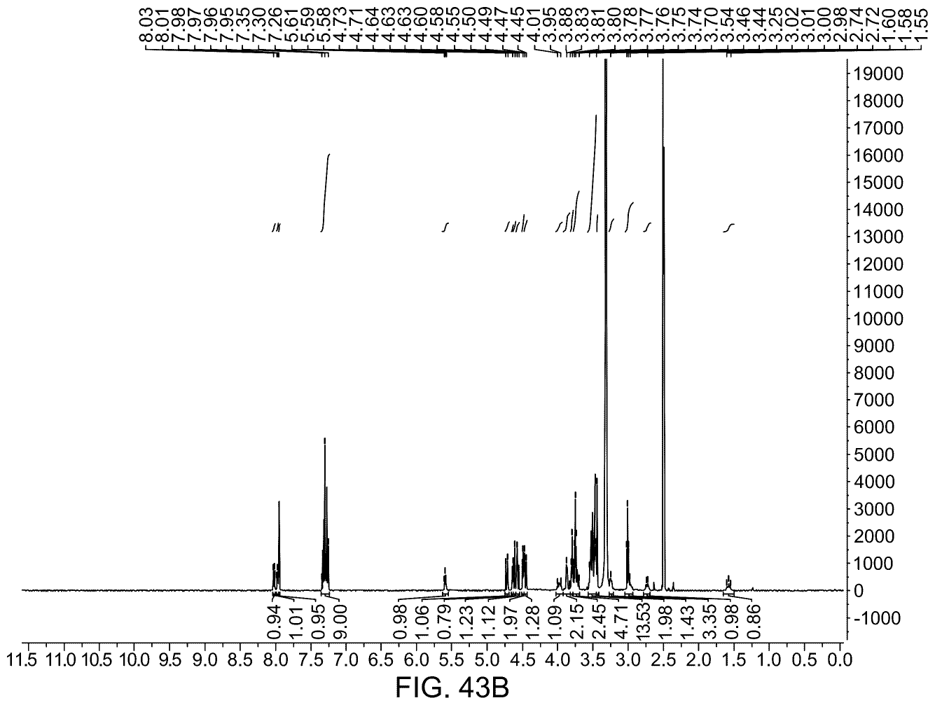

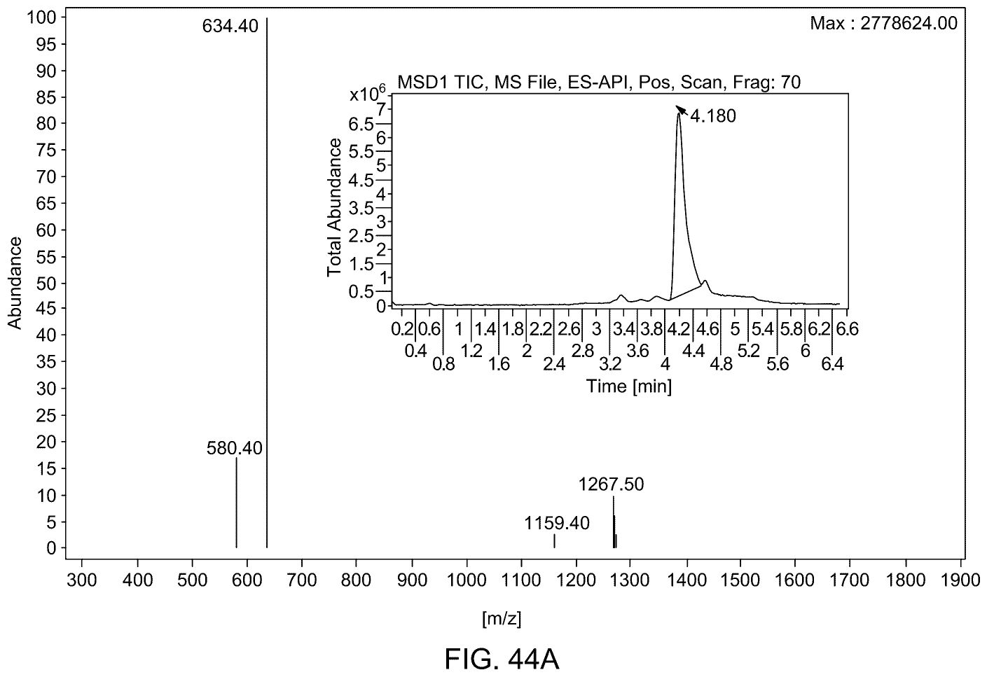

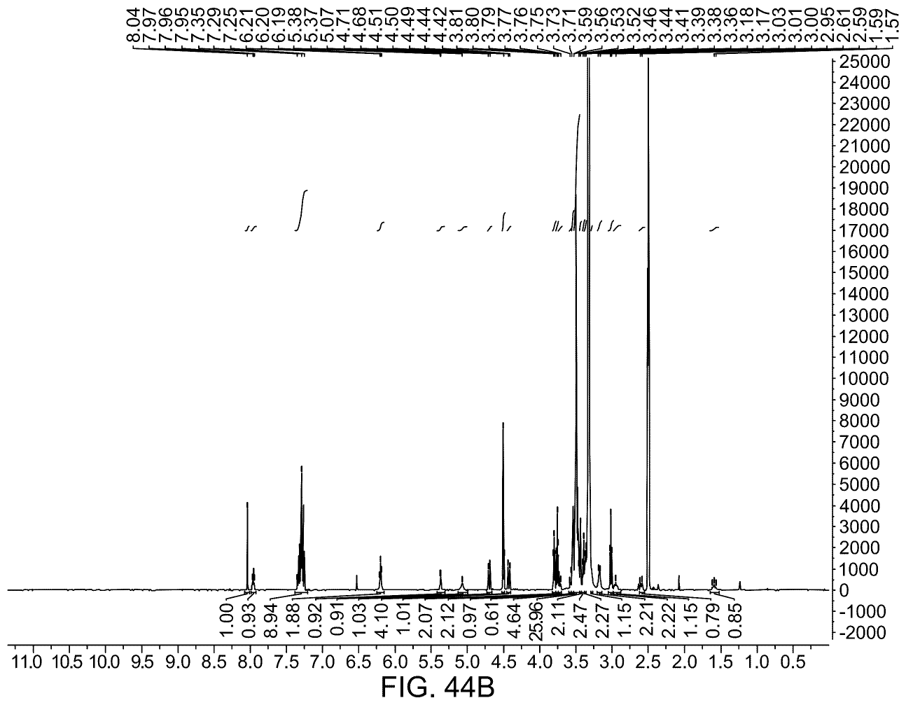

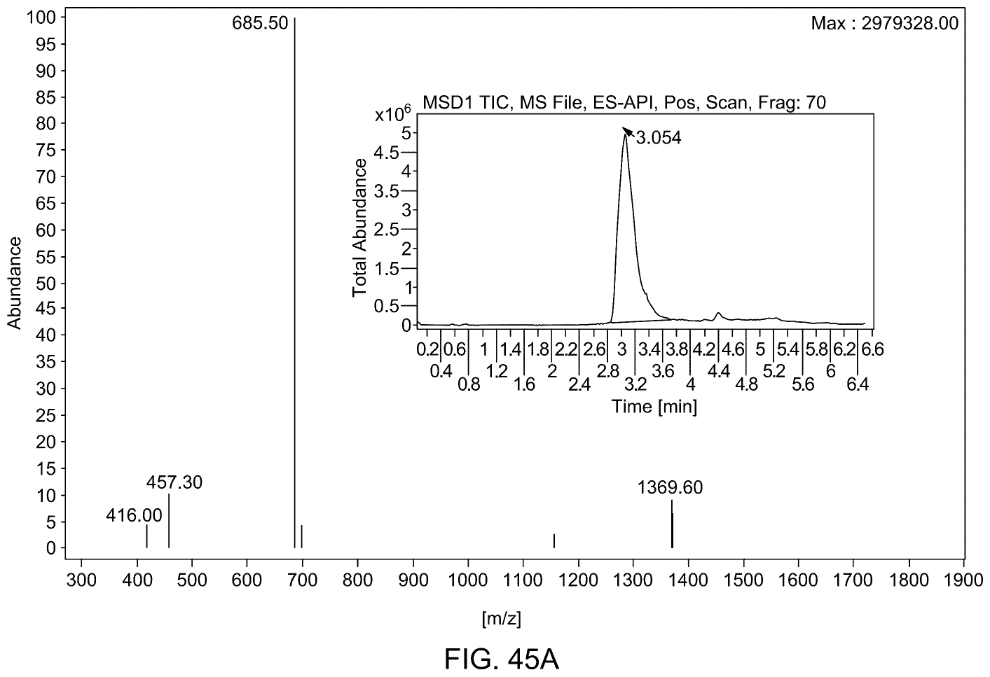

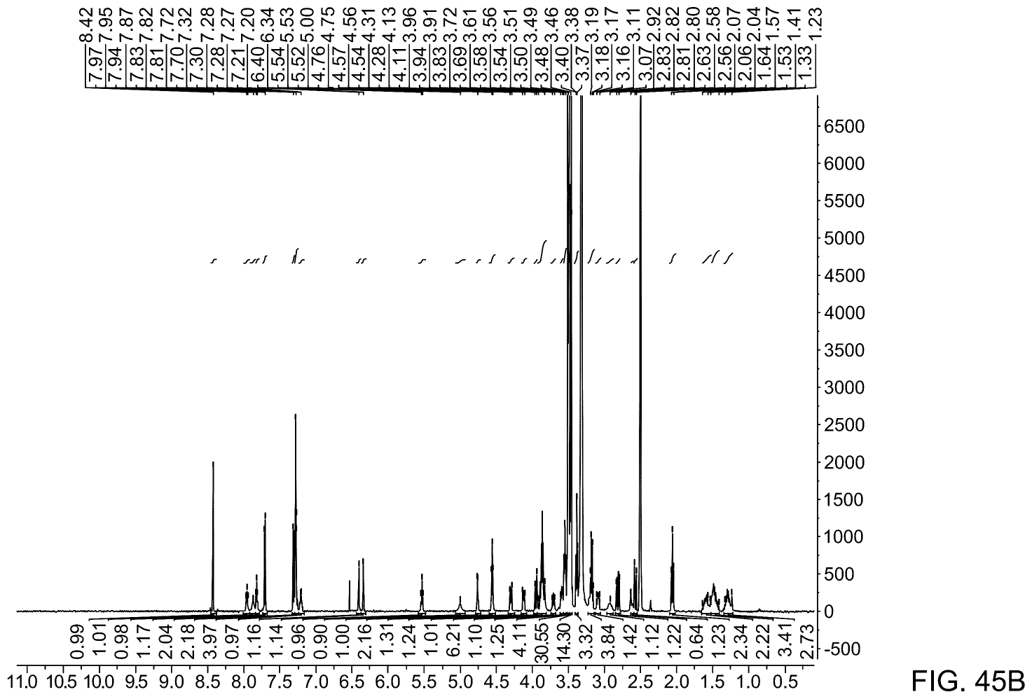

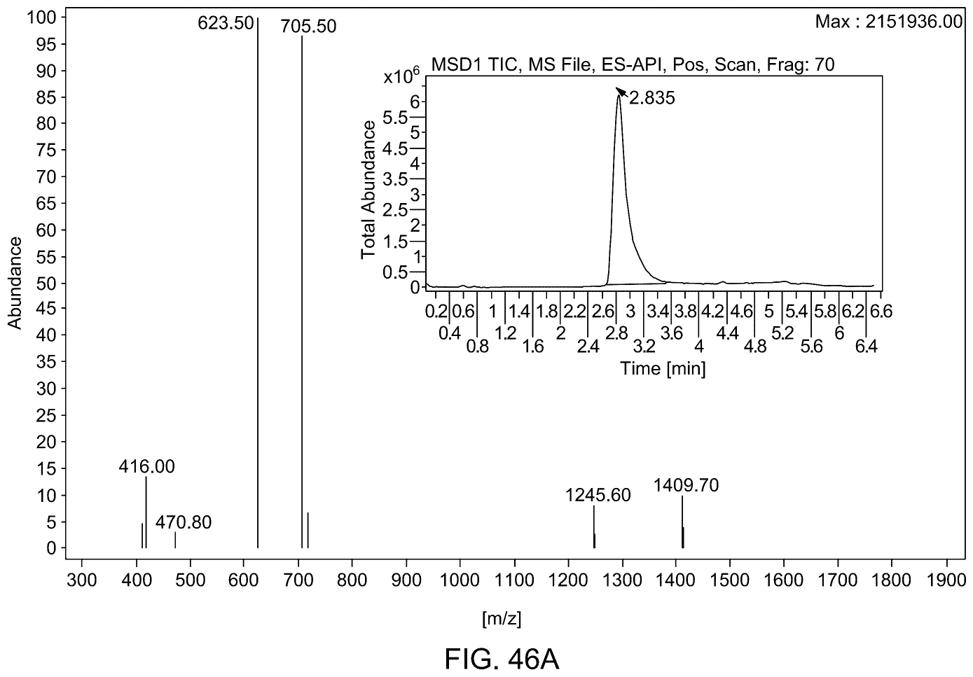

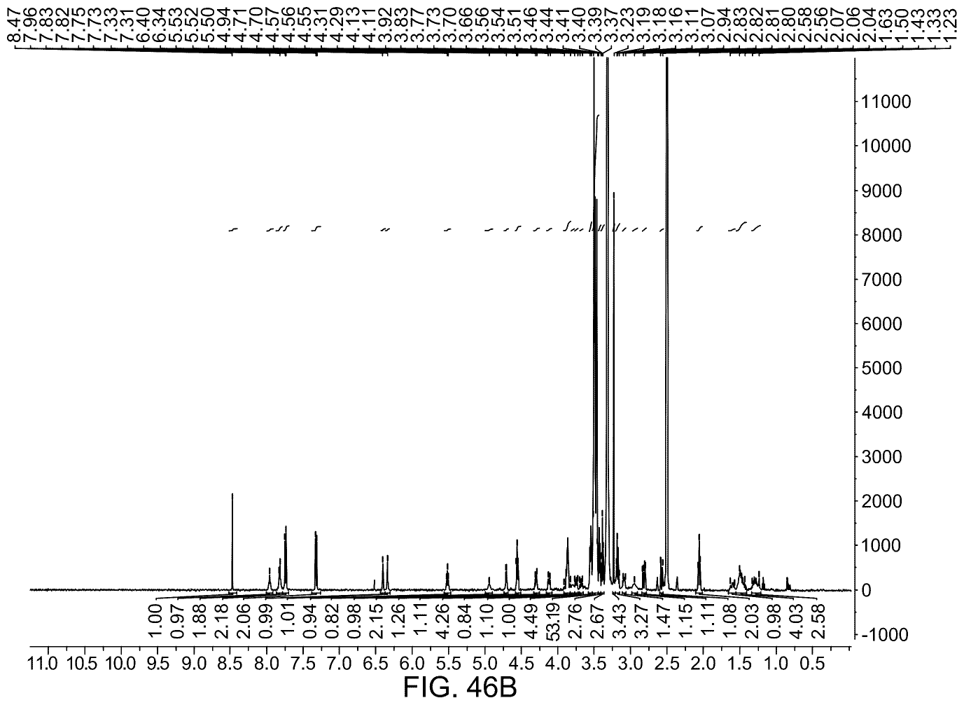

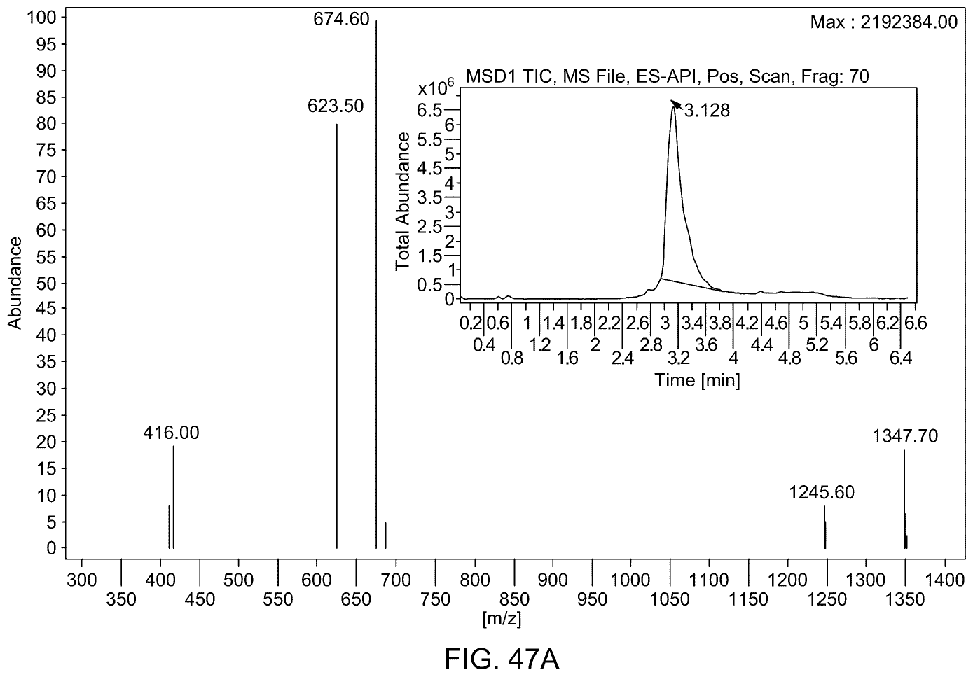

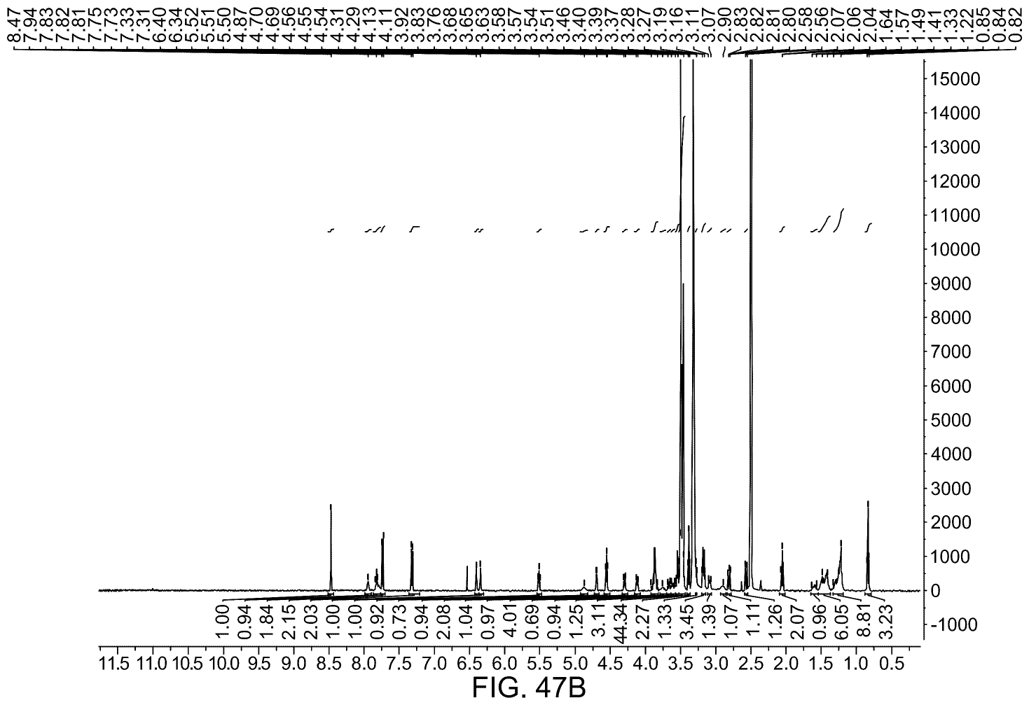

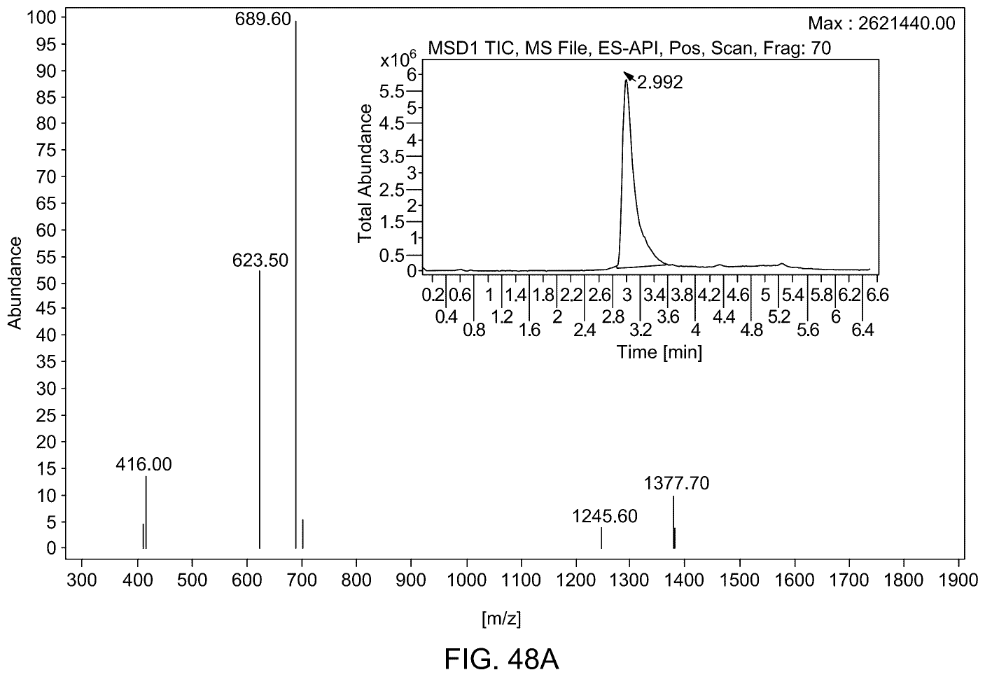

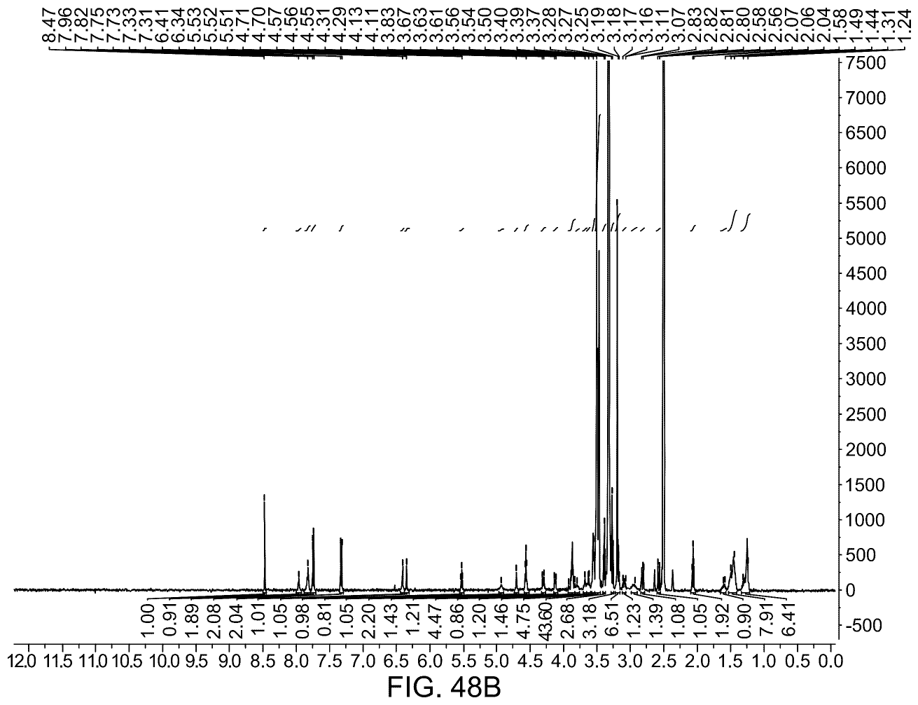

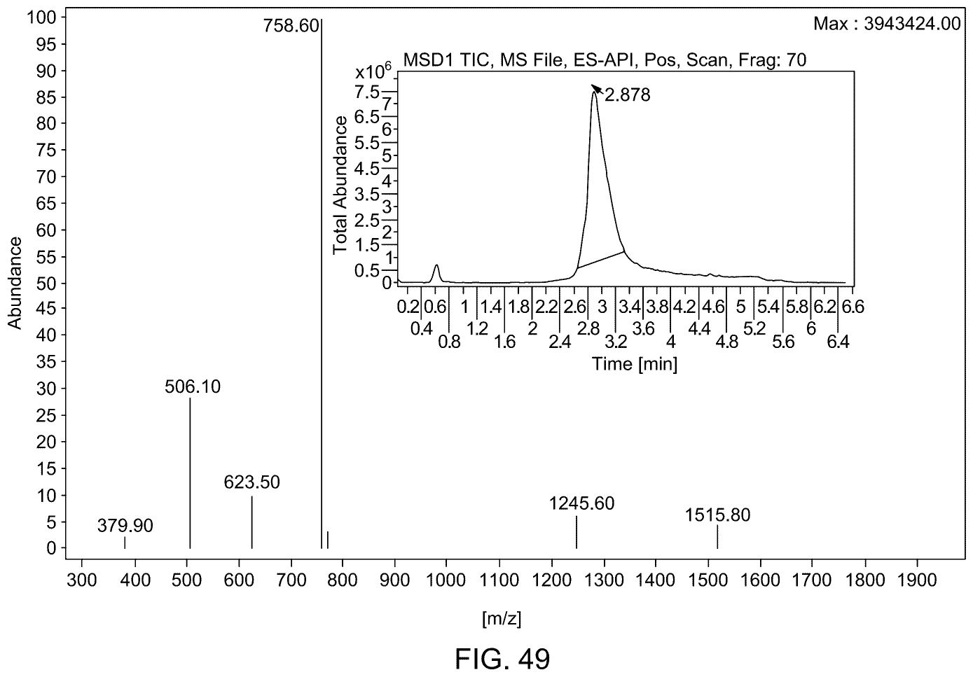

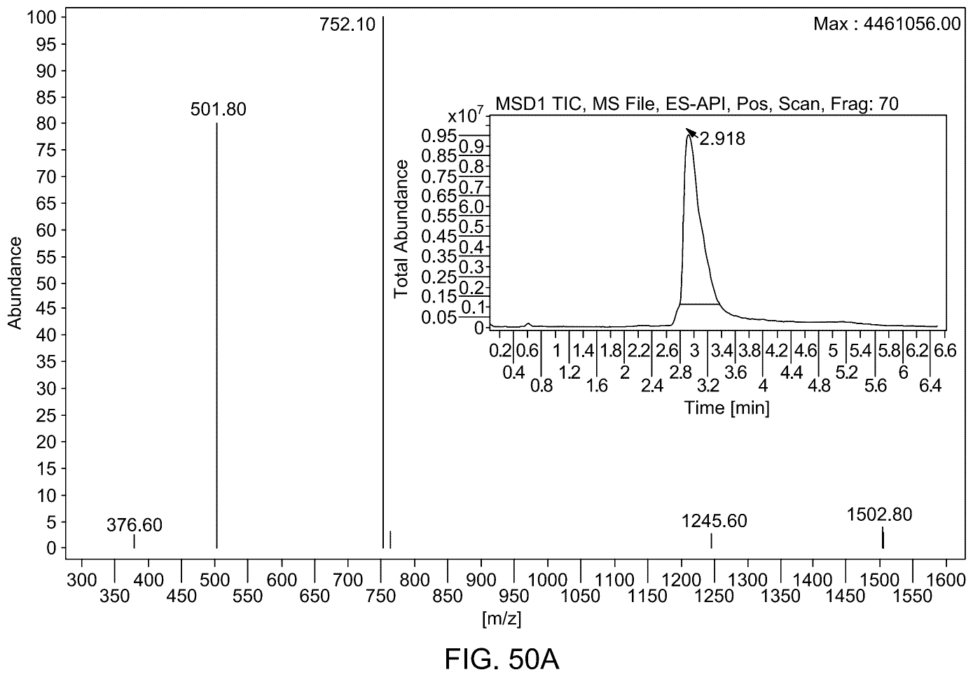

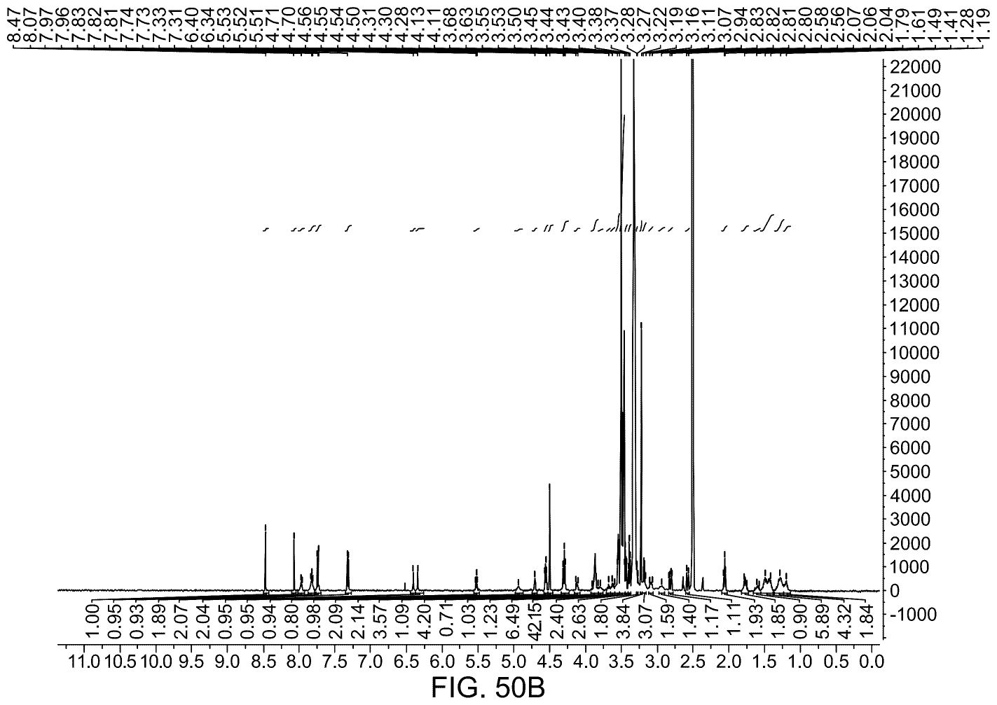

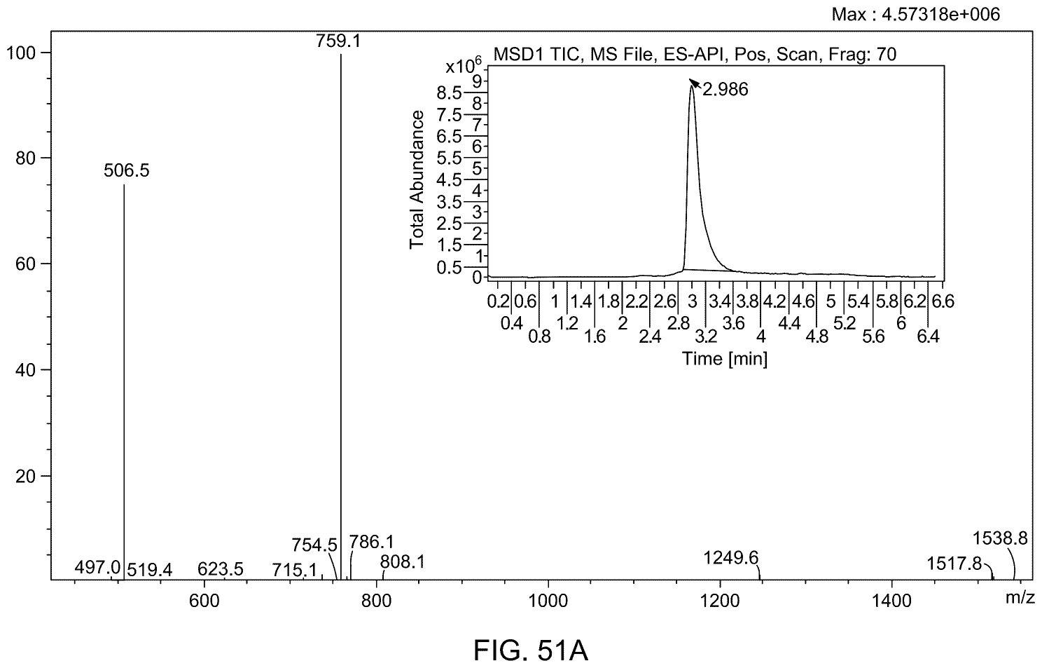

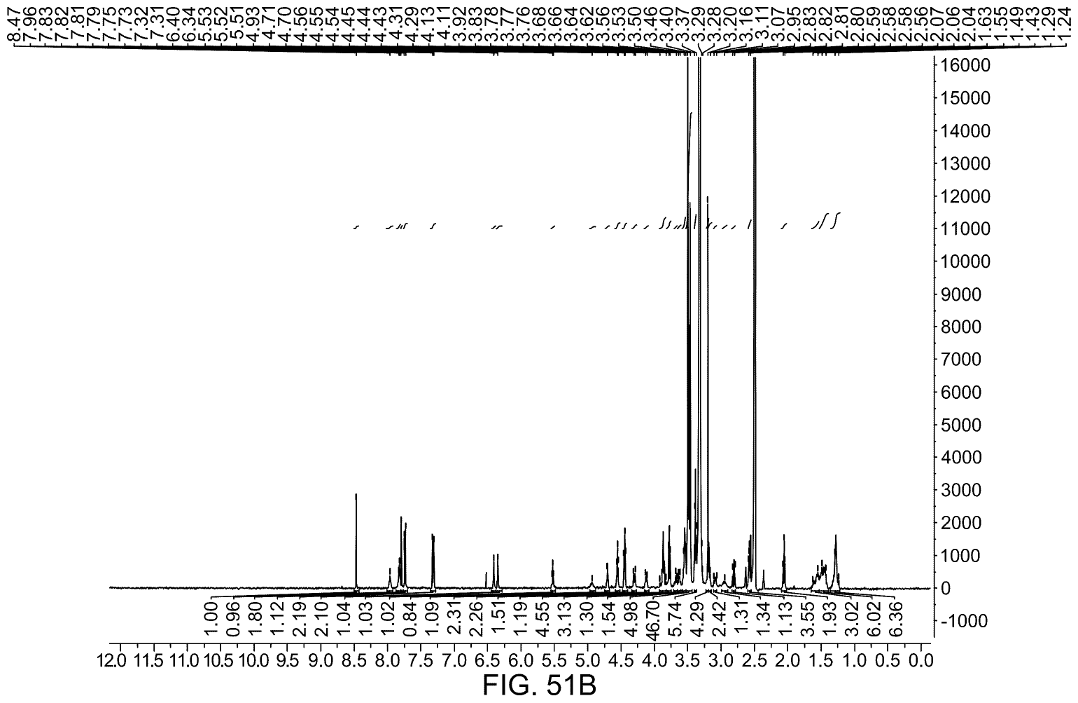

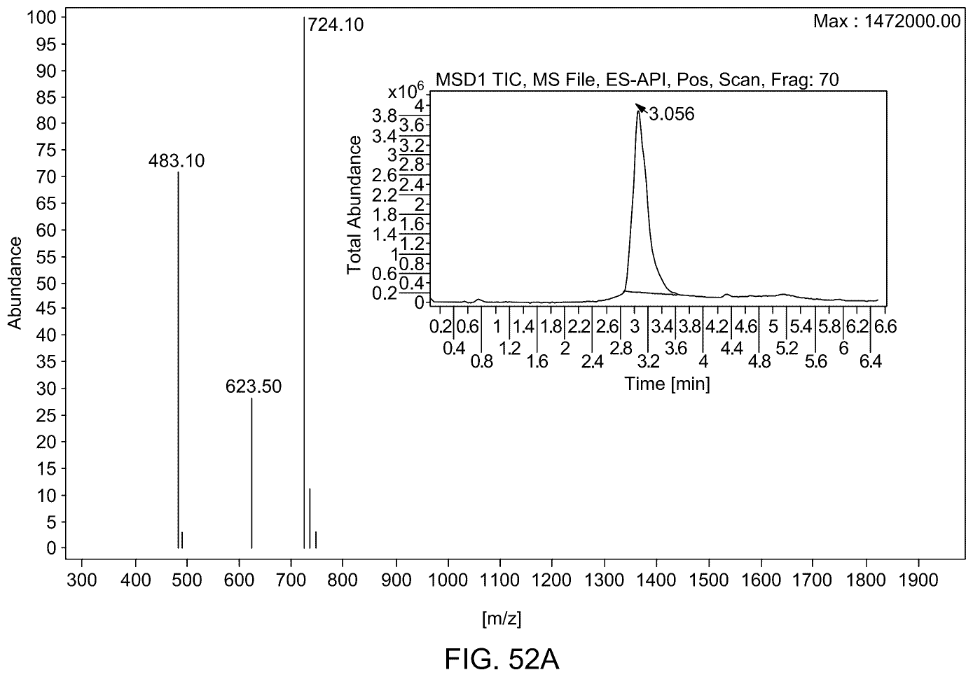

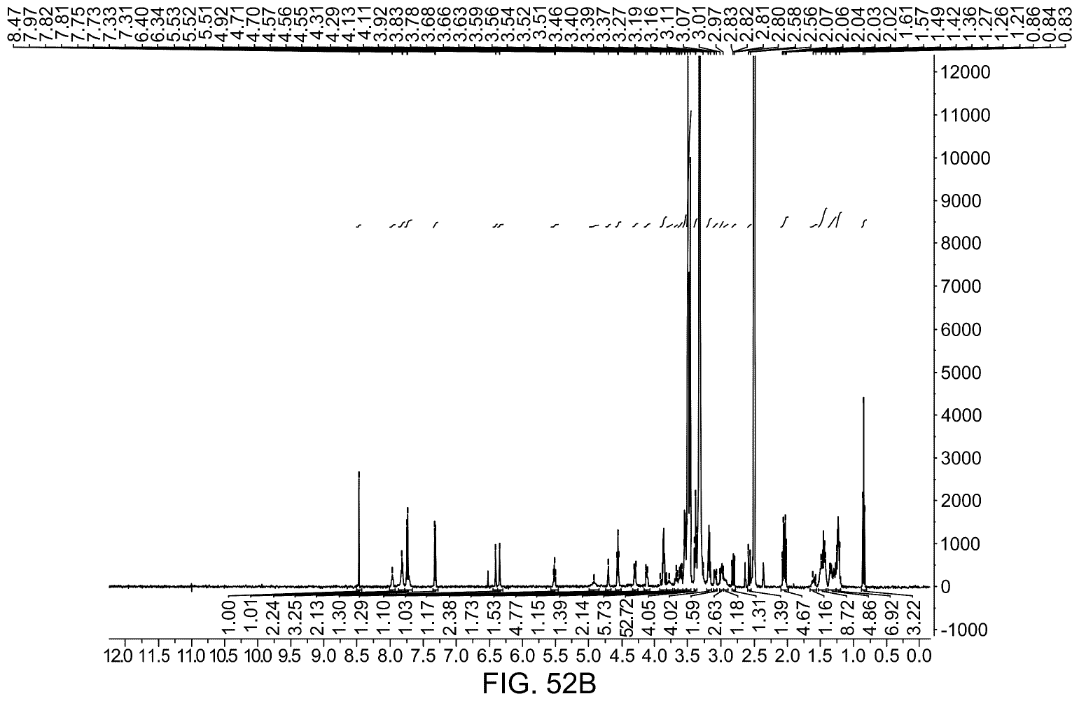

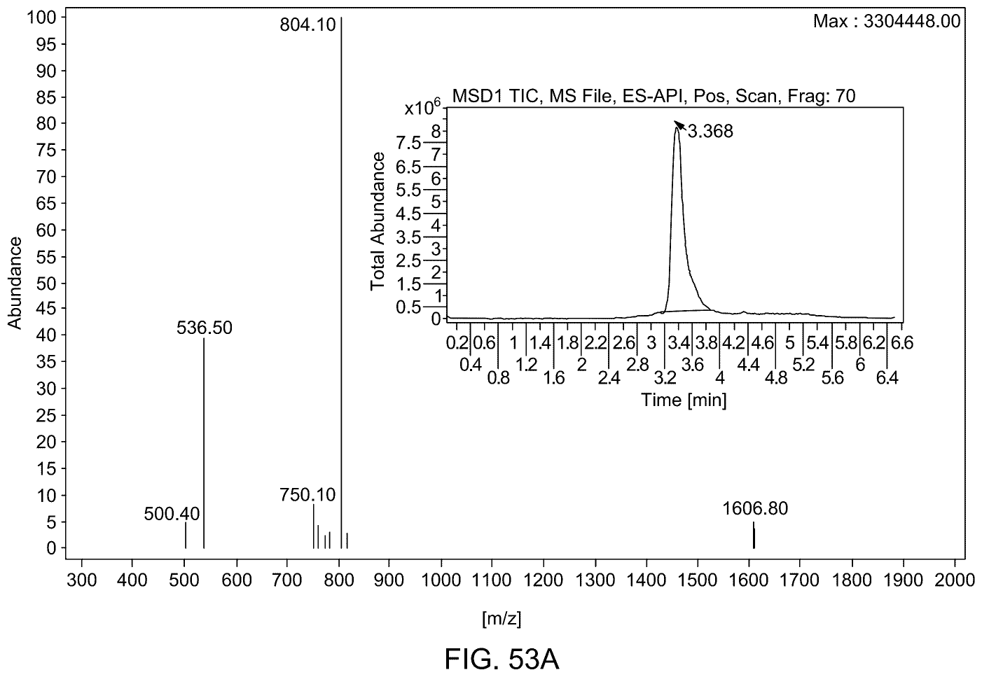

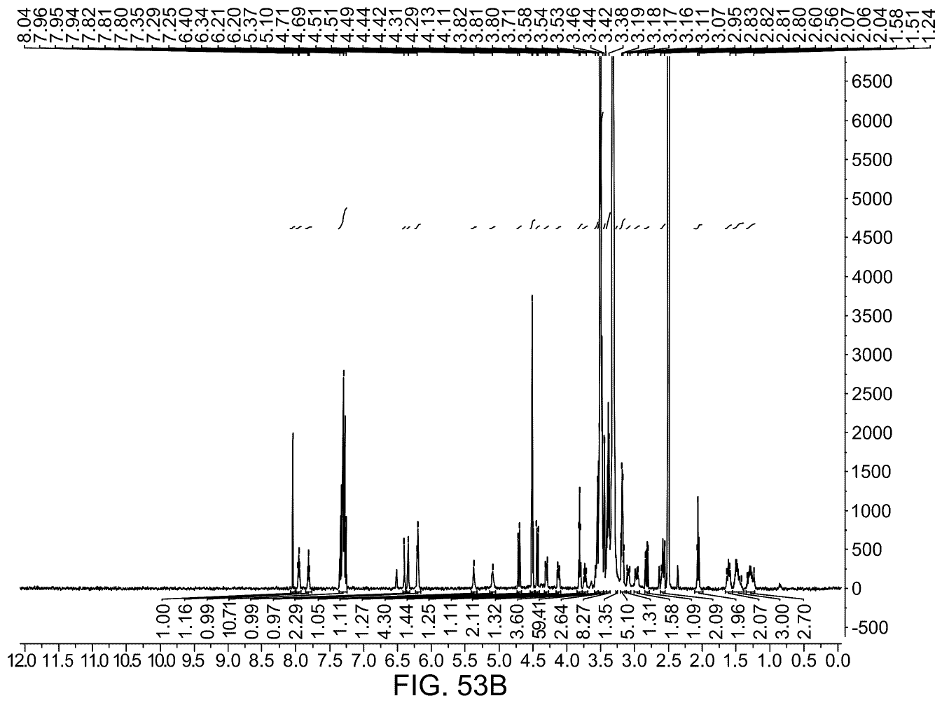

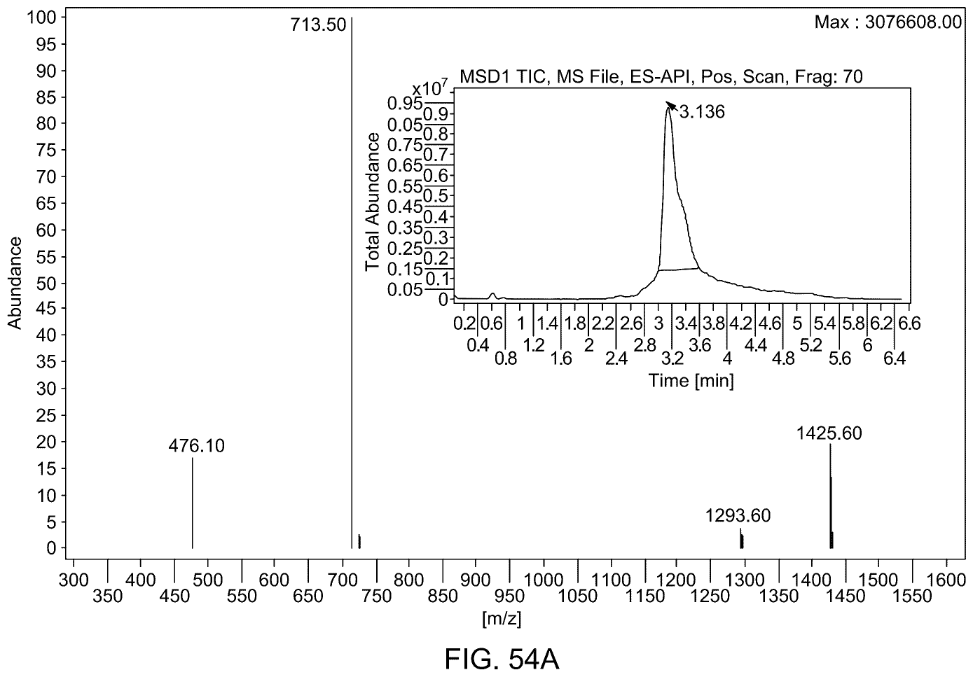

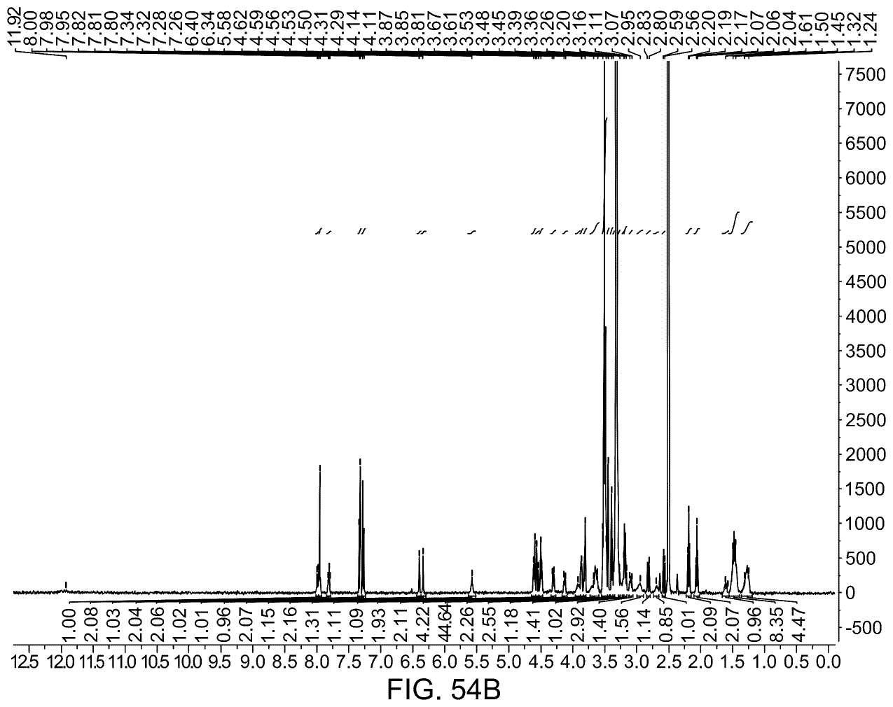

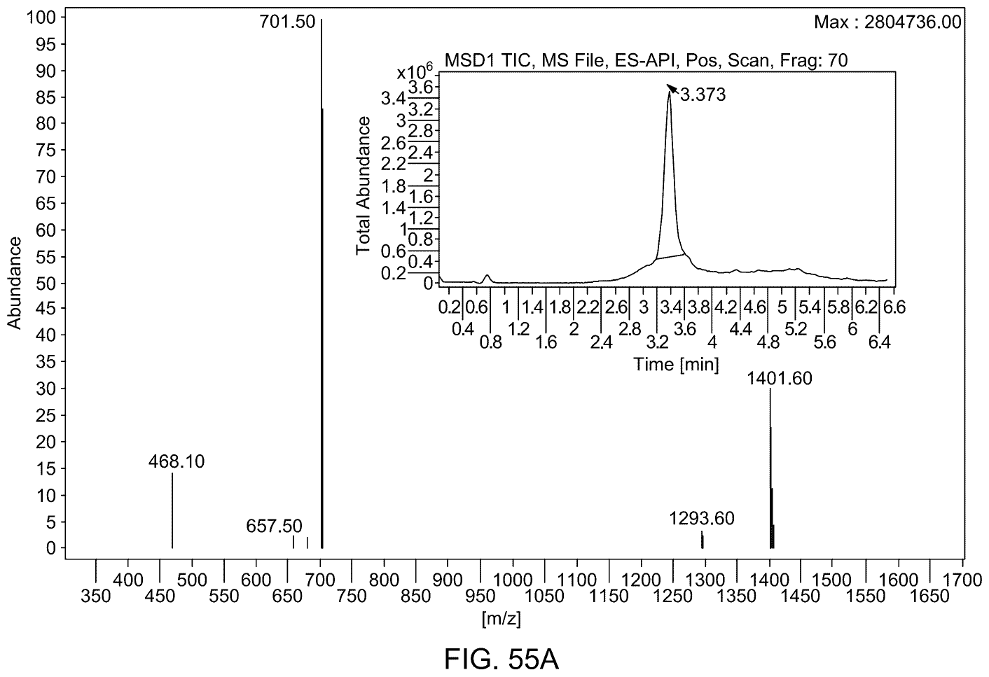

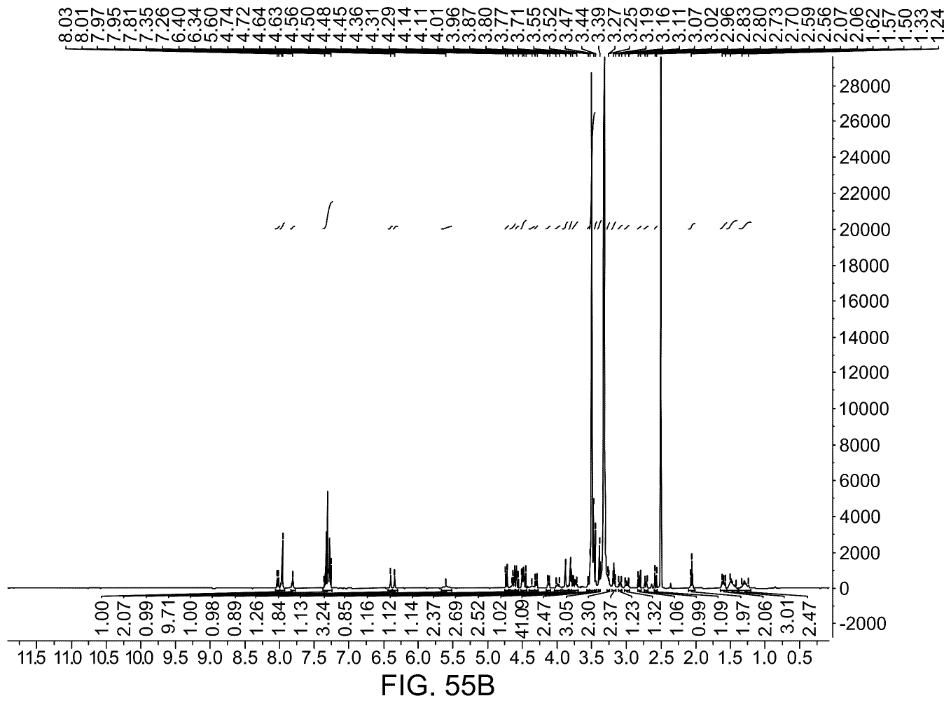

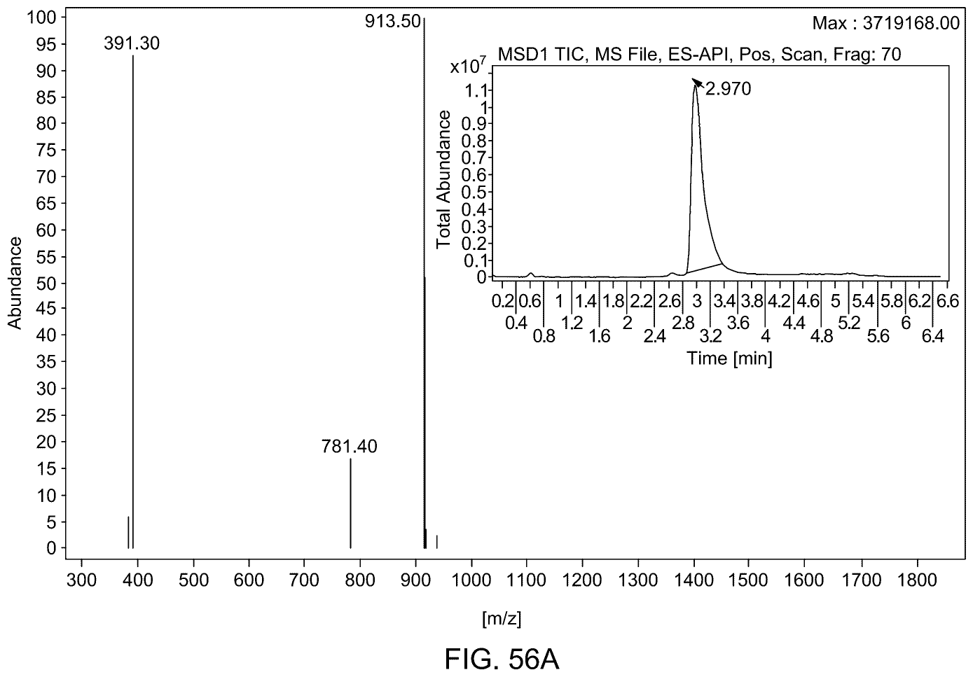

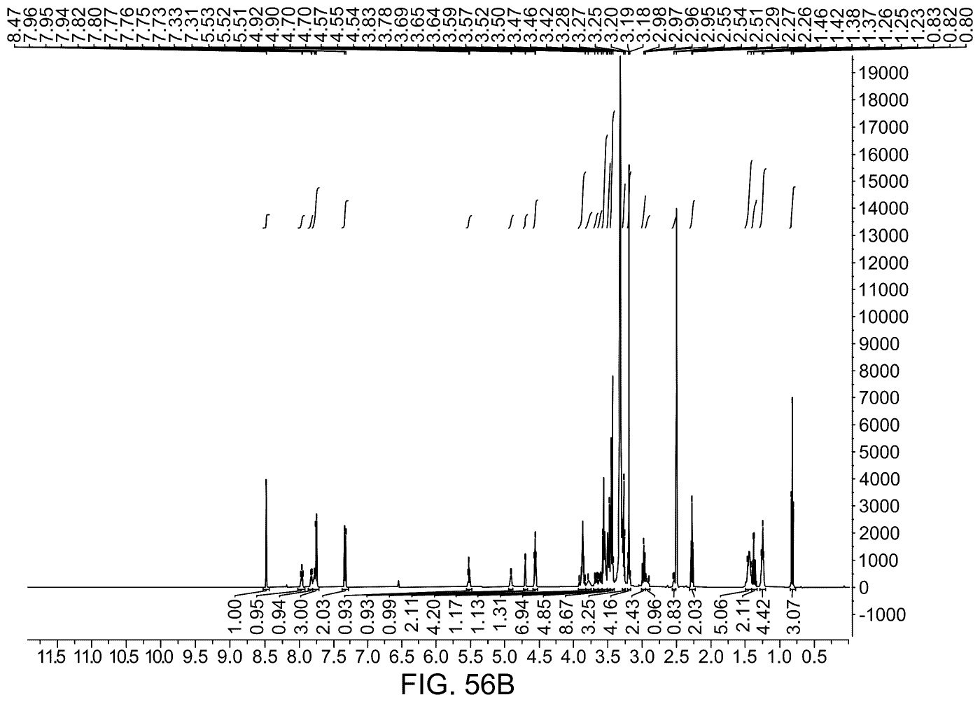

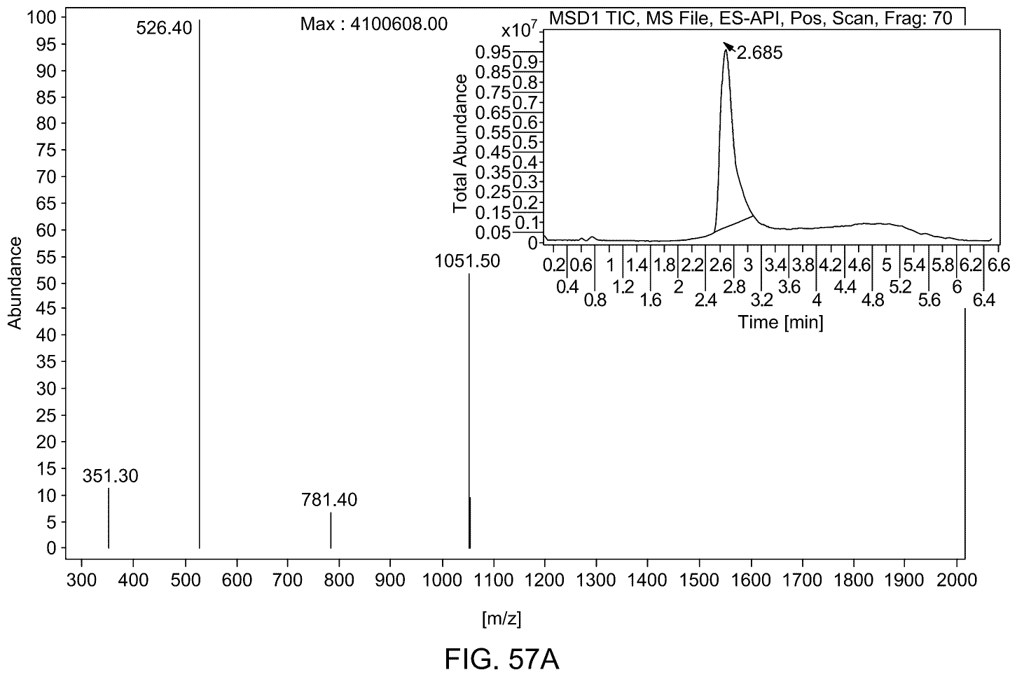

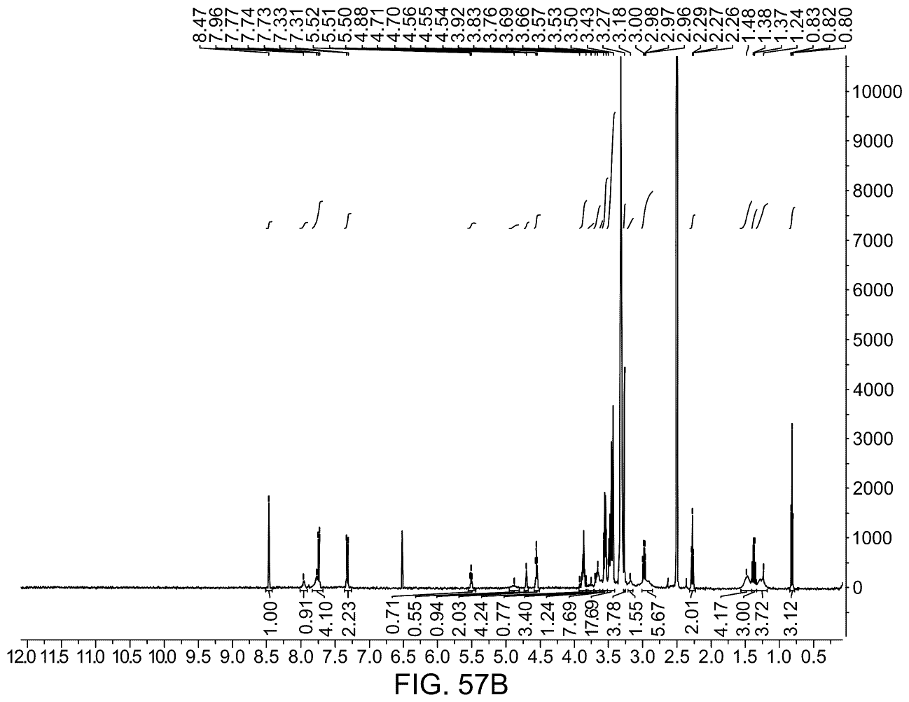

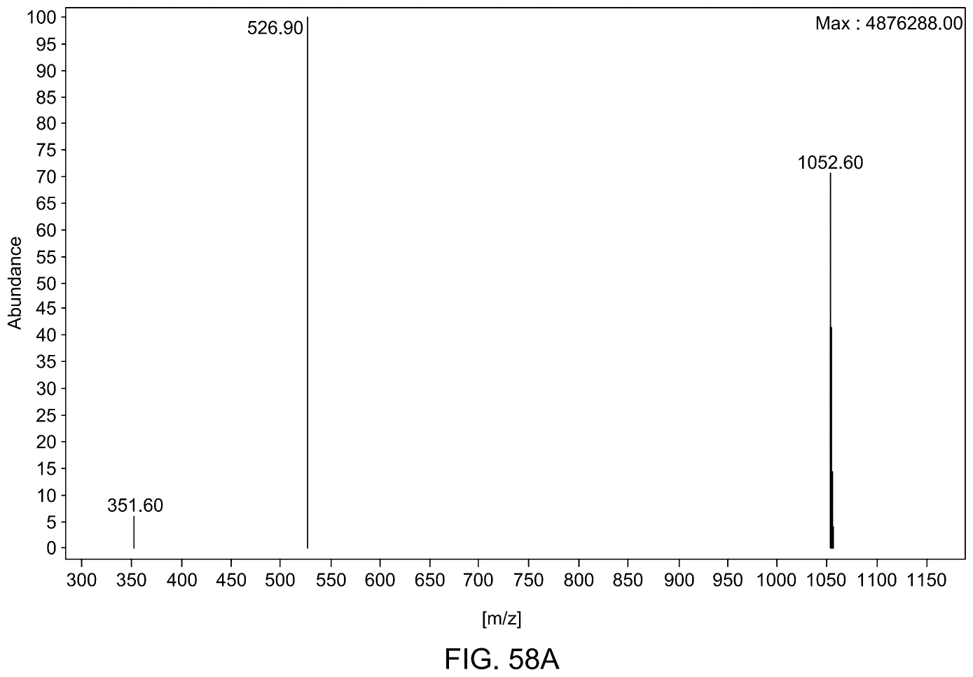

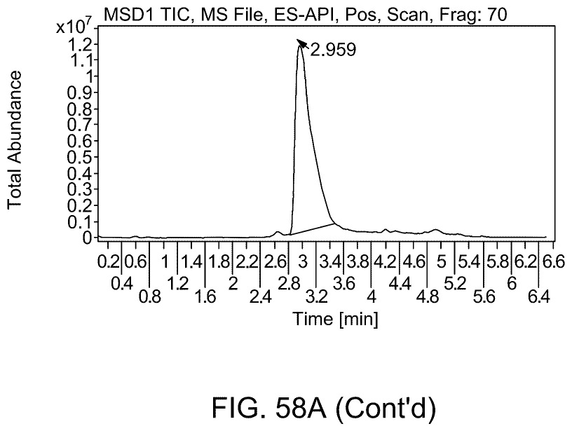

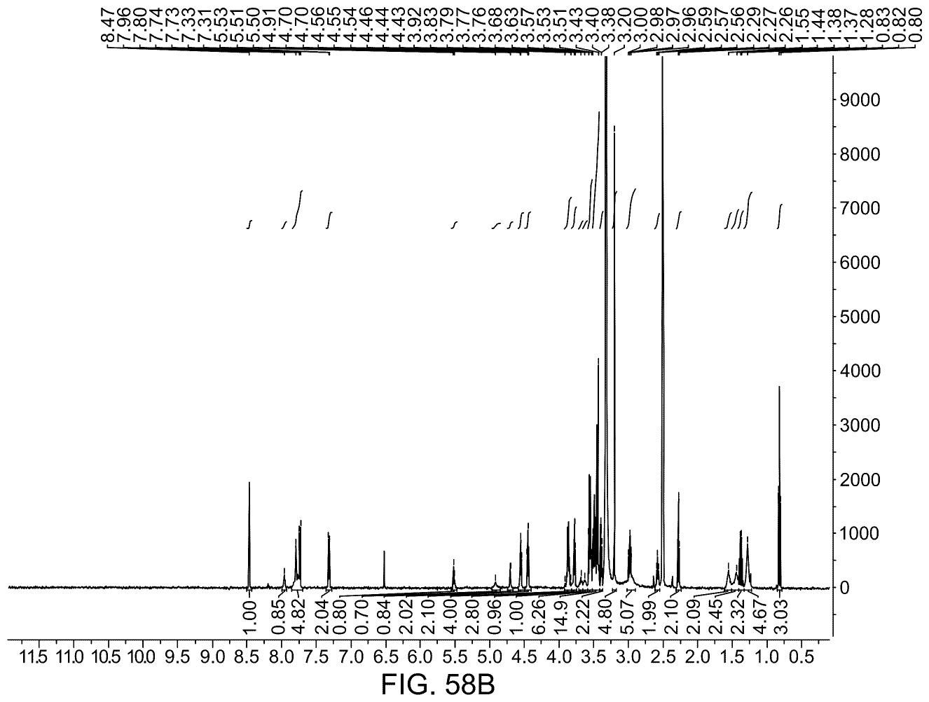

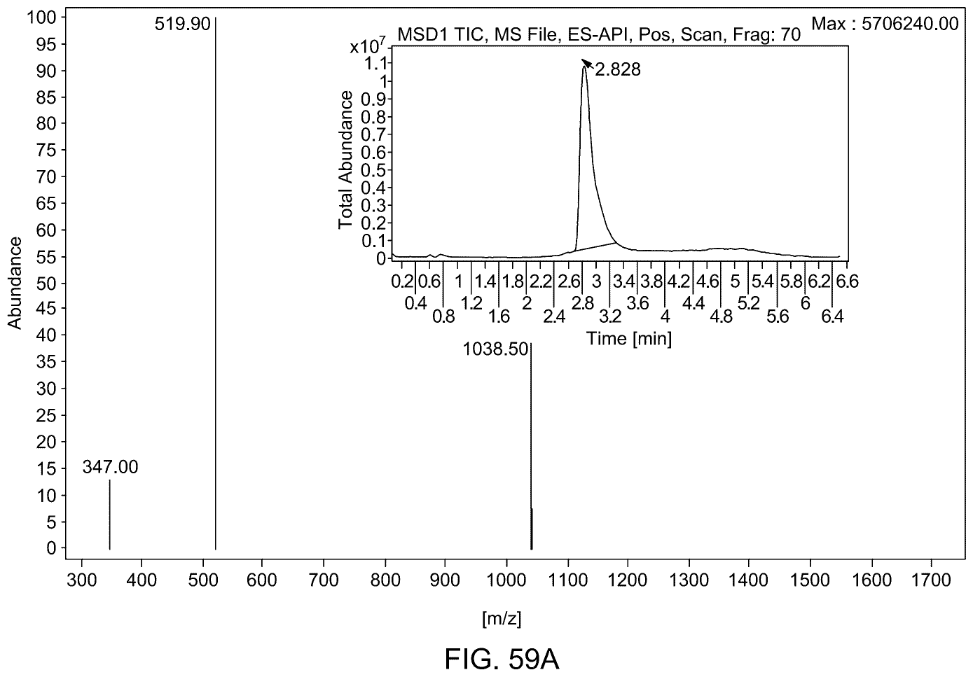

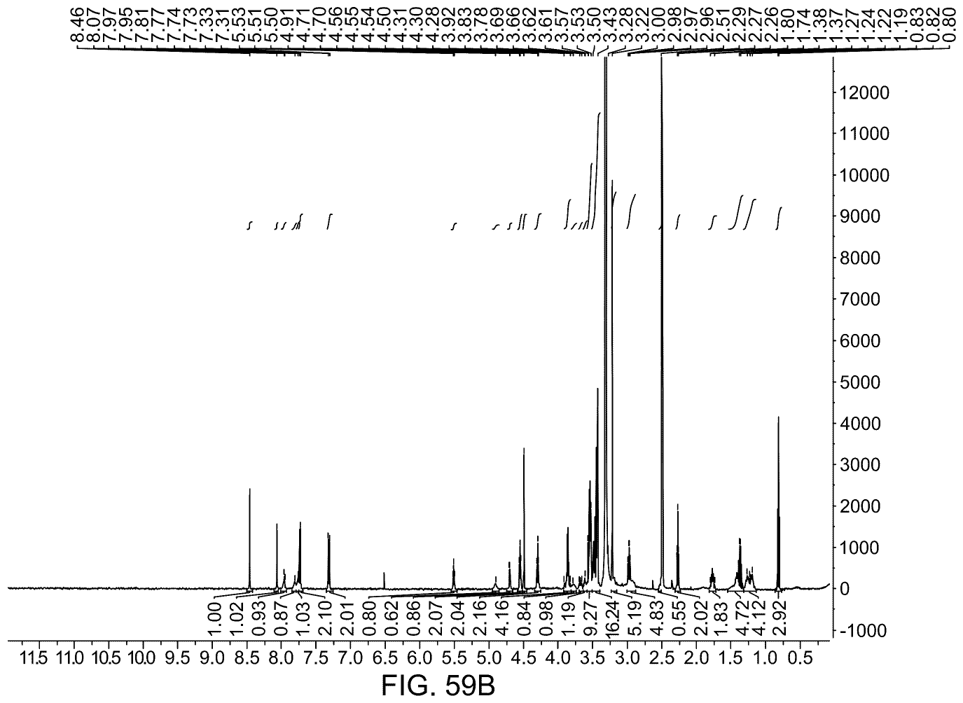

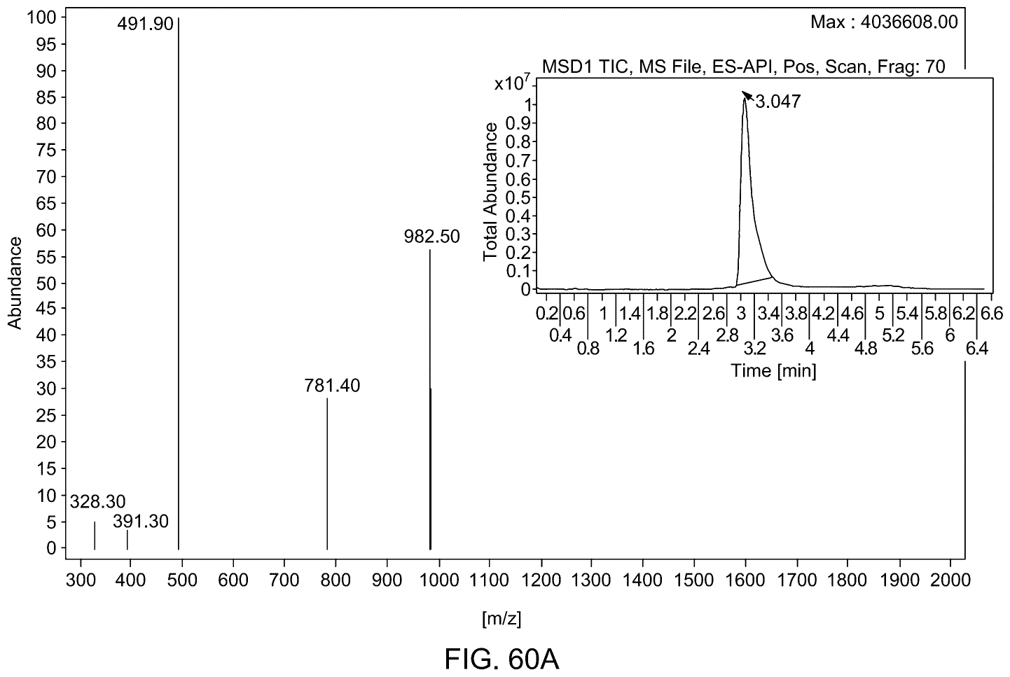

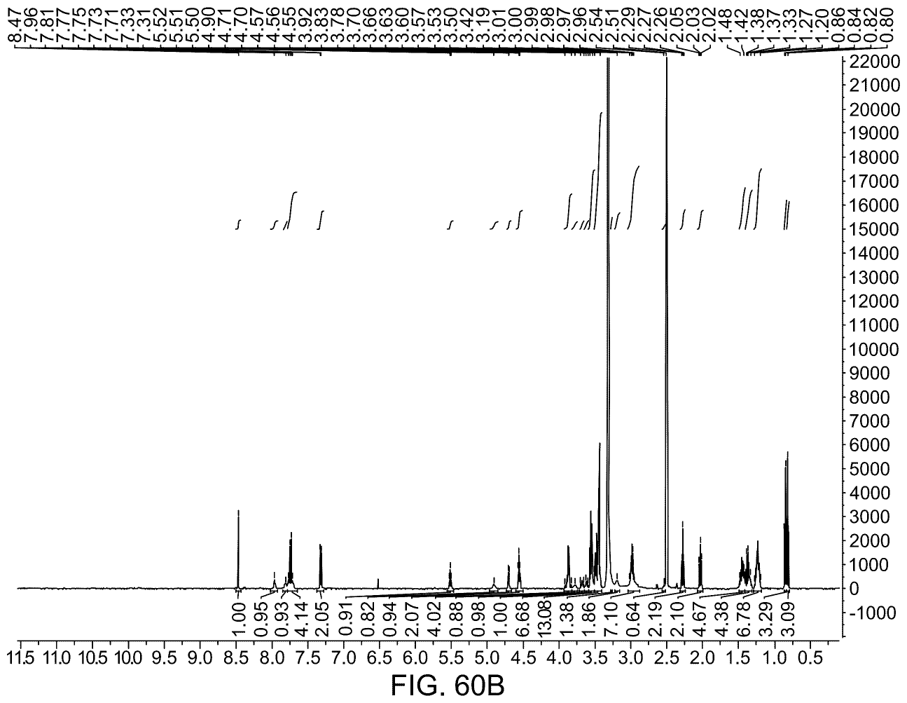

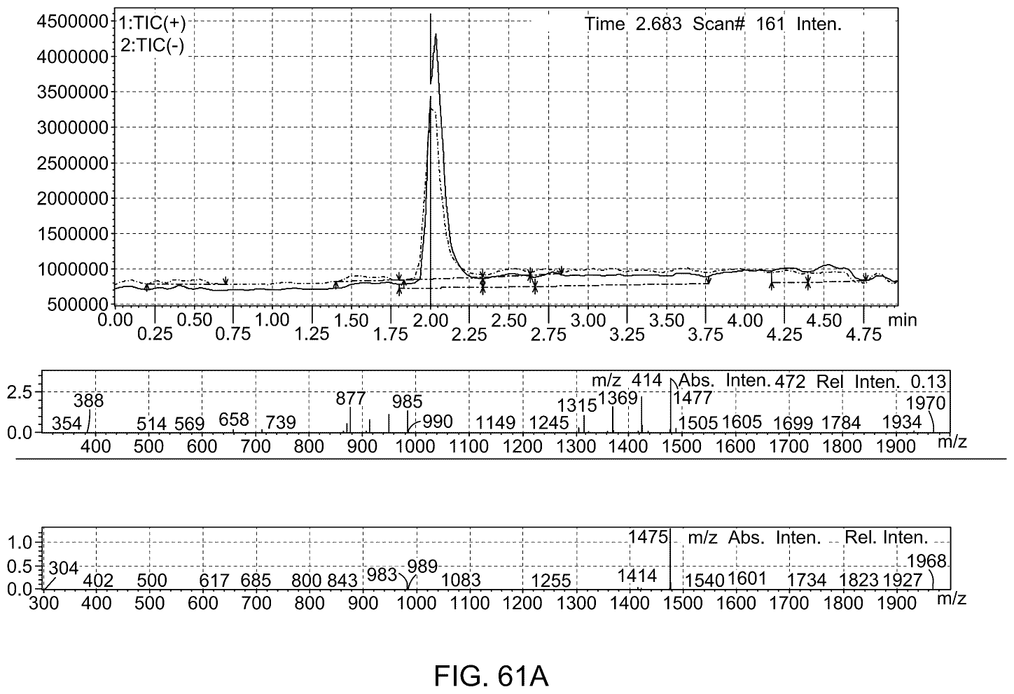

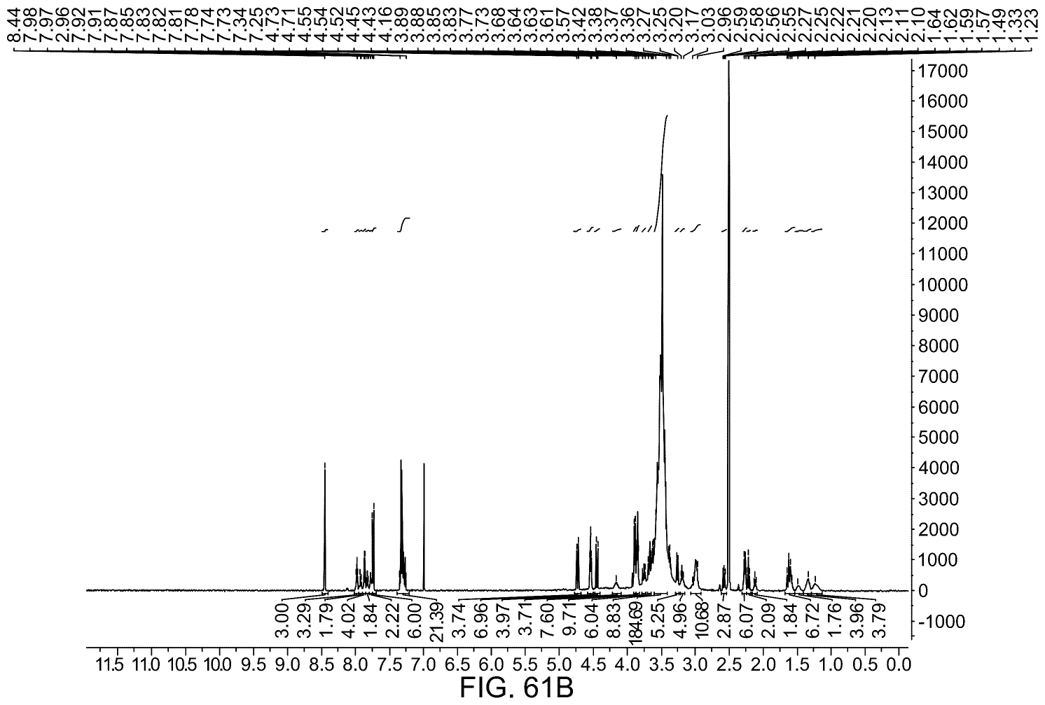

### DETAILED DESCRIPTION

Provided are siglec ligands and siglec conjugates. Also provided are methods of use of such conjugates, as well as pharmaceutical compositions thereof. For example, siglec ligands have particular linkers. In some embodiments, siglec conjugates include a biologically active substance that is covalently bonded to the linker of the siglec ligands. For instance, the biologically active substance can be a biotherapeutic or an autoantigen. The linkers can have advantageous properties that improve the technical qualities of the corresponding conjugates.

Before the present invention is described in greater detail, it is to be understood that this invention is not limited to particular embodiments described, as such may vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials may now be described. Any and all publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. It is further noted that the claims may be drafted to exclude any element, e.g., any optional element. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Further, the dates of publication provided may be different from the actual publication dates which may need to be independently confirmed. To the extent the definition or usage of any term herein conflicts with a definition or usage of a term in an application or reference incorporated by reference herein, the instant application shall control.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### Definitions

### Chemical Groups

“Alkyl” refers to a monoradical, branched or linear, non-cyclic, saturated hydrocarbon group. Exemplary alkyl groups include methyl, ethyl, n-propyl, isopropyl, n-butyl, isobutyl, t-butyl, octyl, decyl, cyclopentyl, and cyclohexyl. In some cases the alkyl group has 1 to 24 carbon atoms, e.g. 1 to 12, 1 to 6, or 1 to 3.

“Alkenyl” refers to a monoradical, branched or linear, non-cyclic hydrocarbonyl group that comprises a carbon-carbon double bond. Exemplary alkenyl groups include ethenyl, n-propenyl, isopropenyl, n-butenyl, isobutenyl, octenyl, decenyl, tetradecenyl, hexadecenyl, eicosenyl, and tetracosenyl.

“Alkynyl” refers to a monoradical, branched or linear, non-cyclic hydrocarbonyl group that comprises a carbon-carbon triple bond. Exemplary alkynyl groups include ethynyl and n-propynyl.

“Cycloalkyl” refers to a monoradical, cyclic, saturated hydrocarbon group. Similarly, “cycloalkenyl” refers to a monoradical and cyclic group having carbon-carbon double bond whereas “cycloalkynyl” refers to a monoradical and cyclic group having carbon-carbon triple bond.

“Heterocyclyl” refers to a monoradical, cyclic group that contains a heteroatom (e.g. O, S, N) as a ring atom and that is not aromatic (i.e. distinguishing heterocyclyl groups from heteroaryl groups). Exemplary heterocyclyl groups include piperidinyl, tetrahydrofuranyl, dihydrofuranyl, and thiocanyl.

“Aryl” refers to an aromatic group containing at least one aromatic ring, wherein each of the atoms in the ring are carbon atoms, i.e. none of the ring atoms are heteroatoms (e.g. O, S, N). In some cases the aryl group has a second aromatic ring, e.g. that is fused to the first aromatic ring. Exemplary aryl groups are phenyl, naphthyl, biphenyl, diphenylether, diphenylamine, and benzophenone.

“Heteroaryl” refers to an aromatic group containing at least one aromatic ring, wherein at least one of the atoms in the aromatic ring is a heteroatom (e.g. O, S, N). Exemplary heteroaryl groups include those obtained from removing a hydrogen atom from pyridine, pyrimidine, furan, thiophene, or benzothiophene.

The term “substituted” refers the removal of one or more hydrogens from an atom (e.g. from a C or N atom) and their replacement with a different group. For instance, a hydrogen atom on a phenyl (—C₆H₅) group can be replaced with a methyl group to form a —C₆H₄CH₃ group. Thus, the —C₆H₄CH₃ group can be considered a substituted aryl group. As another example, two hydrogen atoms from the second carbon of a propyl (—CH₂CH₂CH₃) group can be replaced with an oxygen atom to form a —CH₂C(O)CH₃ group, which can be considered a substituted alkyl group. However, replacement of a hydrogen atom on a propyl (—CH₂CH₂CH₃) group with a methyl group (e.g. giving —CH₂CH(CH₃)CH₃) is not considered a “substitution” as used herein since the starting group and the ending group are both alkyl groups. However, if the propyl group was substituted with a methoxy group, thereby giving a —CH₂CH(OCH₃)CH₃ group, the overall group can no long be considered “alkyl”, and thus is “substituted alkyl”. Thus, in order to be considered a substituent, the replacement group is a different type than the original group. In addition, groups are presumed to be unsubstituted unless described as substituted. For instance, the term “alkyl” and “unsubstituted alkyl” are used interchangeably herein.

Exemplary substituents include alkyl, alkenyl, alkynyl, cycloalkyl, heterocyclyl, aryl, heteroaryl, acyl, alkoxy, amino, azido, carbonyl, carboxy, cyano, ether, halo, hydroxy, nitro, and substituted versions thereof.

In some cases, the substitutions can themselves be further substituted with one or more groups. For example, the group —C₆H₄CH₂CH₃ can be considered as substituted aryl, i.e. an aryl group substituted with the ethyl, which is an alkyl group. Furthermore, the ethyl group can itself be substituted with a pyridyl group to form —C₆H₄CH₂CH₂C₅H₅N, wherein —C₆H₄CH₂CH₂C₅H₅N can also be considered as a substituted aryl group as the term is used herein. In some cases, the substituents are not substituted with any other groups.

Monoradical and multiradical groups are described herein. For example, the methyl group (—CH₃) and the ethyl group (—CH₂CH₃) are monoradical groups. In contrast, exemplary diradical groups include diylmethane (—CH₂—, which is also known as a methylene group) and 1,2-diylethane (—CH₂CH₂—). The term “arylene” refers to the diradical version of an aryl group, e.g. 1,4-diylbenzene refers to a C₆H₄ fragment wherein two hydrogens that are located para to one another are removed and replaced with single bonds to other groups. The terms “alkenylene”, “alkynylene”, “heteroarylene”, and “heterocyclene” are also used herein. The term “diradical connector” is used interchangeably with the term “diradical group”. Exemplary diradical groups include alkyl groups, substituted alkyl groups, polyethylene glycol groups, alkoxy groups, substituted alkoxy groups, arylalkyl groups, and substituted arylalkyl groups.

“Acyl” refers to a group of formula —C(O)R wherein R is alkyl, alkenyl, alkynyl, or substituted versions thereof. For example, the acetyl group has formula —C(O)CH₃. “Carbonyl” refers to a diradical group of formula —C(O)—.

“Alkoxy” refers to a group of formula —O(alkyl). Similar groups can be derived from alkenyl, alkynyl, aryl, heteroaryl, and other groups.

“Amino” refers to the group —NR^{X}R^{Y} wherein RX and R^{Y} are each independently H or a non-hydrogen substituent. Exemplary non-hydrogen substituents include alkyl groups (e.g. methyl, ethyl, and isopropyl) and the carbonyl group.

“Carbonyl” refers to a diradical group of formula —C(O)—.

“Carboxy” is used interchangeably with carboxyl and carboxylate to refer to the —CO₂H group and salts thereof.

“Ether” refers to a diradical group of formula —O—. For instance, if the ether group is connected to an alkyl group, then the overall group is an alkoxy group (e.g. —OCH₃ or methoxy).

If the ether is connected to a carbonyl group, then the overall group is an ester group of formula —OC(O)—.

“Halo” and “halogen” refer to the chloro, bromo, fluoro, and iodo groups.

“Nitro” refers to the group of formula —NO₂.

Unless otherwise specified, reference to an atom is meant to include all isotopes of that atom. For example, reference to H includes ¹H, ²H (i.e. D or deuterium) and ³H (i.e. tritium), and reference to C is includes both ¹²C and all other isotopes of carbon (e.g. ¹³C). Unless specified otherwise, groups include all possible stereoisomers.

“Chemoselective functional group” refers to a functional group that can selectively react with another compatible functional group to form a covalent bond, in some cases, after optional activation of one of the functional groups. Chemoselective functional groups of interest include, but are not limited to, thiols and maleimide or iodoacetamide, amines and carboxylic acids or active esters thereof, as well as groups that can react with one another via Click chemistry, e.g., azide and alkyne groups (e.g., cyclooctyne groups), tetrazine, transcyclooctene, dienes and dienophiles, and azide, sulfur(VI) fluoride exchange chemistry (SuFEX), sulfonyl fluoride, as well as hydroxyl, hydrazido, hydrazino, aldehyde, ketone, azido, alkyne, phosphine, epoxide, succinimide, pentafluorophenyl (PFP) ester, and carboxylic acid (e.g. on a lysine residue).

### Treatment

The terms active agent, active pharmaceutical ingredient, pharmacologically active agent, and drug are used interchangeably herein to refer to a chemical material or compound which, when administered to an organism (human or animal) induces a desired pharmacologic and/or physiologic effect by local and/or systemic action.

The terms “individual,” “host,” “subject,” and “patient” are used interchangeably herein, and refer to an animal, including, but not limited to, human and non-human primates, including simians and humans; rodents, including rats and mice; bovines; equines; ovines; felines; canines; and the like. “Mammal” means a member or members of any mammalian species, and includes, by way of example, canines; felines; equines; bovines; ovines; rodentia, etc. and primates, e.g., non-human primates, and humans. Non-human animal models, e.g., mammals, e.g. non-human primates, murines, lagomorpha, etc. may be used for experimental investigations.

As used herein, the terms “treatment,” “treating,” and the like, refer to obtaining a desired pharmacologic and/or physiologic effect, such as reduction of viral titer. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease (as in liver fibrosis that can result in the context of chronic HCV infection); (b) inhibiting the disease, i.e., arresting its development; and (c) relieving the disease, i.e., causing regression of the disease (e.g., reduction in viral titers).

A “therapeutically effective amount”, a “therapeutically effective dose” or “therapeutic dose” is an amount sufficient to effect desired clinical results (i.e., achieve therapeutic efficacy, achieve a desired therapeutic response, etc.). A therapeutically effective dose can be administered in one or more administrations. For purposes of this disclosure, a therapeutically effective dose of a compositions is an amount that is sufficient, when administered to the individual, to palliate, ameliorate, stabilize, reverse, prevent, slow or delay the progression of a disease state (e.g., cancer, etc.) present in the subject.

As used herein, the terms “determining,” “measuring,” “assessing,” and “assaying” are used interchangeably and include both quantitative and qualitative determinations.

The term “unit dosage form,” as used herein, refers to physically discrete units suitable as unitary dosages for human and animal subjects, each unit containing a predetermined quantity of a compound (e.g., an aminopyrimidine compound, as described herein) calculated in an amount sufficient to produce the desired effect in association with a pharmaceutically acceptable diluent, carrier or vehicle. The specifications for unit dosage forms depend on the particular compound employed and the effect to be achieved, and the pharmacodynamics associated with each compound in the host.

A “pharmaceutically acceptable excipient,” “pharmaceutically acceptable diluent,” “pharmaceutically acceptable carrier,” and “pharmaceutically acceptable adjuvant” means an excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. “A pharmaceutically acceptable excipient, diluent, carrier and adjuvant” as used in the specification and claims includes both one and more than one such excipient, diluent, carrier, and adjuvant.

As used herein, a “pharmaceutical composition” is meant to encompass a composition suitable for administration to a subject, such as a mammal, especially a human. In general a “pharmaceutical composition” is sterile, and preferably free of contaminants that are capable of eliciting an undesirable response within the subject (e.g., the compound(s) in the pharmaceutical composition is pharmaceutical grade). Pharmaceutical compositions can be designed for administration to subjects or patients in need thereof via a number of different routes of administration including oral, buccal, rectal, parenteral, intraperitoneal, intradermal, intracheal, intramuscular, subcutaneous, and the like.

The terms “co-administration” and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

### Siglec Ligands and Conjugates

The term “siglec” as used herein refers to cell surface proteins that binds to a sialic acid group. siglecs are commonly found on the surface of leukocytes.

There are 14 different mammalian siglecs, which are expressed on different types of leukocytes and which may exert inhibitory or activating effects on the cells on which they are expressed depending on whether they comprise an inhibitory motif or activating motif. Siglecs show distinct binding preferences for different sialic acids, and the type of linkage and type of underlying sugar also affect recognition of sialic acids. (Varki, A. and Crocker, P. R. (2009) I-type lectins. In Essentials of Glycobiology (2nd edn) (Varki, A. et al., eds), pp. 459-474, Cold Spring Harbor Laboratory Press; Crocker, P. R. et al. (2007) Nat. Rev. Immunol. 7, 255-266). Together, this provides for an array of alternative siglec ligands that may be deployed to modulate an immune response to a biotherapeutic.

Provided by the present disclosure are sialic acid-binding immunoglobulin-type lectin ligands, which are also referred to herein as “siglec ligands”.

### Siglec Ligands of Formula (I)

Provided are siglec ligands of formula (I):

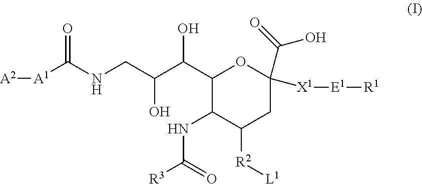

wherein:
A¹ is absent or —(CH₂)ₙ—, wherein n is an integer ranging from 1 to 10;
A² is alkyl, aryl, heteroaryl, cycloalkyl, or a substituted version thereof;
R¹ is H, alkyl, alkoxy, carboxyl, ester, amine, aryl, heteroaryl, cycloalkyl, heterocycle, carbohydrate, phosphate, squaramide, or a substituted version thereof;
R² is O, S, —NR⁴ R⁵, or —CR⁴ R⁵; wherein R⁴ and R⁵ are each independently selected from the group consisting of H, alkyl, substituted alkyl, alkenyl, substituted alkenyl, alkynyl, substituted alkynyl, cycloalkyl, substituted cycloalkyl, heterocyclyl, substituted heterocyclyl, aryl, substituted aryl, heteroaryl, and substituted heteroaryl;
R³ is OR^{3′}, NHR^{3′}, alkyl, —CH₂OR^{3′}, —CH₂NHR^{3′}, wherein R^{3′} is H, alkyl, aryl, heteroaryl, cycloalkyl, or a substituted version thereof;
X¹ is O, S, —CH₂—, —CH(OH)—, or triazole;
E¹ is absent, —(CH₂CH₂O)_{g}— or —(CH₂)_{g}—, wherein g is an integer ranging from 1 to 10;
L¹ is a linker;
a salt thereof, or a stereoisomer thereof.

As discussed above, the siglec ligand can have formula (I), or it can be salt of formula (I) or a stereoisomer of formula (I). For example, R³ is described as an amino group, such as unsubstituted —NH₂. If R³ is —NH₃⁺ Cl⁻, then the compound would be a salt of formula (I). By “stereoisomer thereof”, it means that a composition could include primarily a particular stereoisomer. For example, O group in hexane ring is a chiral center, and a composition could include primarily one of the two possible stereoisomers at that chiral center.

In some cases, A² is alkyl or substituted alkyl. For instance, A² has the formula CR⁶R⁷R⁸, wherein R⁶, R⁷, and R⁸ are each independently selected from the group consisting of H, alkyl, substituted alkyl, alkenyl, substituted alkenyl, alkynyl, substituted alkynyl, cycloalkyl, substituted cycloalkyl, heterocyclyl, substituted heterocyclyl, aryl, substituted aryl, heteroaryl, and substituted heteroaryl.

In some cases, R¹ is alkyl or substituted alkyl. For instance, A² has the formula CR⁶R⁷R⁸, wherein R⁶, R⁷, and R⁸ are each independently selected from the group consisting of H, alkyl, substituted alkyl, alkenyl, substituted alkenyl, alkynyl, substituted alkynyl, cycloalkyl, substituted cycloalkyl, heterocyclyl, substituted heterocyclyl, aryl, substituted aryl, heteroaryl, and substituted heteroaryl.

In some cases, R³ is alkyl or substituted alkyl. For instance, A² has the formula CR⁶R⁷R⁸, wherein R⁶, R⁷, and R⁸ are each independently selected from the group consisting of H, alkyl, substituted alkyl, alkenyl, substituted alkenyl, alkynyl, substituted alkynyl, cycloalkyl, substituted cycloalkyl, heterocyclyl, substituted heterocyclyl, aryl, substituted aryl, heteroaryl, and substituted heteroaryl.

In some embodiments of formula (I), A¹ is —CH₂—.

In some embodiments of formula (I), A² is aryl, or a substituted version thereof.

In some embodiments of formula (I), X¹ is O.

In some embodiments of formula (I), E¹ is —CH₂—.

In some embodiments of formula (I), R¹ is phenyl.

In some embodiments of formula (I), R² is O.

In some embodiments of formula (I), R³ is —CH₂OH.

### Siglec Ligands of Formula (II)

Provided are siglec ligands of formula (II):

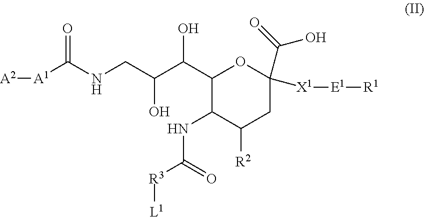

wherein:
A¹ is absent or —(CH₂)ₙ—, wherein n is an integer ranging from 1 to 10;
A² is alkyl, aryl, heteroaryl, cycloalkyl, or a substituted version thereof;
R¹ is H, alkyl, alkoxy, carboxyl, ester, amine, aryl, heteroaryl, cycloalkyl, heterocycle, carbohydrate, phosphate, squaramide, or a substituted version thereof;
R² is OH;
R³ is —O—, —NH—, —CH₂—, —CH₂O—, or —CH₂NH—;
X¹ is O, S, —CH₂—, —CH(OH)—, or triazole;
E¹ is absent, —(CH₂CH₂O)_{g}— or —(CH₂)_{g}—, wherein g is an integer ranging from 1 to 10;
L¹ is a linker;
a salt thereof, or a stereoisomer thereof.

As discussed above, the siglec ligand can have formula (II), or it can be salt of formula (II) or a stereoisomer of formula (II). For example, R² is described as hydroxy group, such as —OH. If R² is —O⁻Na⁺, then the compound would be a salt of formula (II). By “stereoisomer thereof”, it means that a composition could include primarily a particular stereoisomer. For example, O group in hexane ring is a chiral center, and a composition could include primarily one of the two possible stereoisomers at that chiral center.

In some cases, A² is alkyl or substituted alkyl. For instance, A² has the formula CR⁶R⁷R⁸, wherein R⁶, R⁷, and R⁸ are each independently selected from the group consisting of H, alkyl, substituted alkyl, alkenyl, substituted alkenyl, alkynyl, substituted alkynyl, cycloalkyl, substituted cycloalkyl, heterocyclyl, substituted heterocyclyl, aryl, substituted aryl, heteroaryl, and substituted heteroaryl.

In some cases, R¹ is alkyl or substituted alkyl. For instance, A² has the formula CR⁶R⁷R⁸, wherein R⁶, R⁷, and R⁸ are each independently selected from the group consisting of H, alkyl, substituted alkyl, alkenyl, substituted alkenyl, alkynyl, substituted alkynyl, cycloalkyl, substituted cycloalkyl, heterocyclyl, substituted heterocyclyl, aryl, substituted aryl, heteroaryl, and substituted heteroaryl.

In some embodiments of formula (II), A¹ is —CH₂—.

In some embodiments of formula (II), A² is aryl, or a substituted version thereof.

In some embodiments of formula (II), X¹ is O.

In some embodiments of formula (II), E¹ is —CH₂—.

In some embodiments of formula (II), R¹ is phenyl.

In some embodiments of formula (II), R² is OH.

In some embodiments of formula (II), R³ is —CH₂O—.

### Siglec Ligands of Formula (III)

Provided are siglec ligands of formula (III):

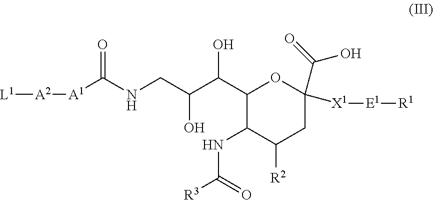

wherein:
A¹ is absent or —(CH₂)ₙ—, wherein n is an integer ranging from 1 to 10;
A² is alkyl, aryl, heteroaryl, cycloalkyl, or a substituted version thereof;
R¹ is H, alkyl, alkoxy, carboxyl, ester, amine, aryl, heteroaryl, cycloalkyl, heterocycle, carbohydrate, phosphate, squaramide, or a substituted version thereof;
R² is OH;
R³ is OR^{3′}, NHR^{3′}, alkyl, —CH₂OR^{3′}, —CH₂NHR^{3′}, wherein R^{3′} is H, alkyl, aryl, heteroaryl, cycloalkyl, or a substituted version thereof;
X¹ is O, S, —CH₂—, —CH(OH)—, or triazole;
E¹ is absent, —(CH₂CH₂O)_{g}— or —(CH₂)_{g}—, wherein g is an integer ranging from 1 to 10;
L¹ is a linker;
a salt thereof, or a stereoisomer thereof.

As discussed above, the siglec ligand can have formula (III), or it can be salt of formula (II) or a stereoisomer of formula (III). For example, R² is described as hydroxy group, such as —OH. If R² is —O⁻Na⁺, then the compound would be a salt of formula (III). By “stereoisomer thereof”, it means that a composition could include primarily a particular stereoisomer. For example, O group in hexane ring is a chiral center, and a composition could include primarily one of the two possible stereoisomers at that chiral center.

In some cases, A² is alkyl or substituted alkyl. For instance, A² has the formula CR⁶R⁷R⁸, wherein R⁶, R⁷, and R⁸ are each independently selected from the group consisting of H, alkyl, substituted alkyl, alkenyl, substituted alkenyl, alkynyl, substituted alkynyl, cycloalkyl, substituted cycloalkyl, heterocyclyl, substituted heterocyclyl, aryl, substituted aryl, heteroaryl, and substituted heteroaryl.

In some cases, R¹ is alkyl or substituted alkyl. For instance, A² has the formula CR⁶R⁷R⁸, wherein R⁶, R⁷, and R⁸ are each independently selected from the group consisting of H, alkyl, substituted alkyl, alkenyl, substituted alkenyl, alkynyl, substituted alkynyl, cycloalkyl, substituted cycloalkyl, heterocyclyl, substituted heterocyclyl, aryl, substituted aryl, heteroaryl, and substituted heteroaryl.

In some cases, R³ is alkyl or substituted alkyl. For instance, A² has the formula CR⁶R⁷R⁸, wherein R⁶, R⁷, and R⁸ are each independently selected from the group consisting of H, alkyl, substituted alkyl, alkenyl, substituted alkenyl, alkynyl, substituted alkynyl, cycloalkyl, substituted cycloalkyl, heterocyclyl, substituted heterocyclyl, aryl, substituted aryl, heteroaryl, and substituted heteroaryl.

In some embodiments of formula (III), A¹ is —CH₂—.

In some embodiments of formula (III), A² is alkyl, aryl, alkoxy, amine, or a substituted version thereof.

In some embodiments of formula (III), A² is aryl.

In some embodiments of formula (III), X¹ is O.

In some embodiments of formula (III), X¹ is S.

In some embodiments of formula (III), E¹ is absent.

In some embodiments of formula (III), E¹ is —CH₂—.

In some embodiments of formula (III), R¹ is phenyl.

In some embodiments of formula (III), R¹ is substituted phenyl.

In some embodiments of formula (III), R² is OH.

In some embodiments of formula (III), R³ is —CH₂OH.

In some embodiments of formula (I), formula (II) and/or formula (III), A¹ is absent. In some cases, —(CH₂)ₙ—, wherein n is an integer ranging from 1 to 10, for example, A¹ is methyl, ethyl, propyl, butyl, pentyl, hexyl, heptyl, or octyl.

In some embodiments of formula (I), formula (II) and/or formula (III), A² is alkyl, for example, saturated, straight-chain, or branched C₁-C₁₅ alkyl. In some cases, A² is methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1,1-dimethylpropyl, 1-ethylpropyl, hexyl, 1,1-dimethylbutyl, 2,2-dimethylbutyl, 3,3-dimethylbutyl, 1,2-dimethylbutyl, 1,3-dimethylbutyl, 2,3-dimethylbutyl, 1-methylpentyl, 2-methylpentyl, 3-methylpentyl, 4-methylpentyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, heptyl, or octyl.

In some cases, A² is a substituted version of alkyl, for example substituted with C₁-C₈ alkyl, halogen, amino, C₁-C₈ alkyloxy, cyano, nitro, aryl, heteroaryl, or C₃-C₈ cycloalkyl.

In some embodiments of formula (I), formula (II) and/or formula (III), A² is an aryl, for example, C₆-C₁₄ aryl. In some cases, A² is phenyl, 1-naphthyl, 2-naphthyl, 1-biphenylene, or 2-biphenylene. In some cases, A² is a substituted version of an aryl, for example substituted with C₁-C₈ alkyl, halogen, hydroxy, amino, C₁-C₈ alkyloxy, C₁-C₈ haloalkyl, cyano, nitro, aryl, heteroaryl, or C₃-C₈ cycloalkyl.

In some embodiments of formula (I), formula (II) and/or formula (III), A² is a heteroaryl for example, 5-10 membered heteroaryl containing one or more heteroatoms selected from S, N, O or SO₂. In some cases, A² is furyl, thienyl, pyrrolyl, pyrazolyl, oxazolyl, pyridinyl, pyrimidinyl, pyrazinyl or 4H-thieno[3,2-c]chromene. In some cases, A² is a substituted version of a heteroaryl, for example substituted with C₁-C₈ alkyl, halogen, amino, C₁-C₈ alkyloxy, cyano, nitro, aryl, heteroaryl, or C₃-C₈ cycloalkyl.

In some embodiments of formula (I), formula (II) and/or formula (III), A² is a cycloalkyl, for example, C₃-C₈ cycloalkyl. In some cases, A² is cyclopropyl, cyclobutyl, cyclopentyl, cyclohexyl, cycloheptyl or cyclooctyl.

### Linkers

As discussed above, in the terminology of formula (I), (II) and (III), the “linker” referred to above is the L¹ moiety. For instance, the linker L¹ is represented by —(X²)ₘ—Z, wherein each X² is independently selected from —(CH₂)ₕ—, —O—, —CH₂O—, —(CH₂CH₂O)ₕ—, —C(O)NH—, —C≡C—, and —CH₂C≡C—, triazole, carboxyl, ester, or substituted version thereof, wherein m is 0 or an integer ranging from 1 to 10, and h is an integer ranging from 1 to 10; and Z is a chemoselective functional group.

In some embodiments, the chemoselective functional group is selected from thiol, maleimide, iodoacetamide, amine, carboxyl, ester, triazole, tetrazine, transcyclooctene, diene, dienophile, sulfonyl fluoride, hydroxyl, hydrazido, hydrazino, aldehyde, ketone, azido, alkyne, phosphine, epoxide, succinimide, and pentafluorophenyl (PFP) ester, or a substituted version thereof.

In some embodiments, each X² is independently selected from —(CH₂)ₕ—, —O—, —CH₂O—, —(CH₂CH₂O)ₕ—, —C(O)NH—, or a substituted version thereof, wherein m is 0 or an integer ranging from 1 to 10, and h is an integer ranging from 1 to 10.

In some embodiments, Z is alkyne, azido, hydroxyl, carboxyl, maleimide, thiol, amine, or a pentafluorophenyl (PFP) ester.

As such, the linker L¹ can have advantageous properties that improve the technical qualities of a conjugate.

In some embodiments, the siglec ligands can be selected from:

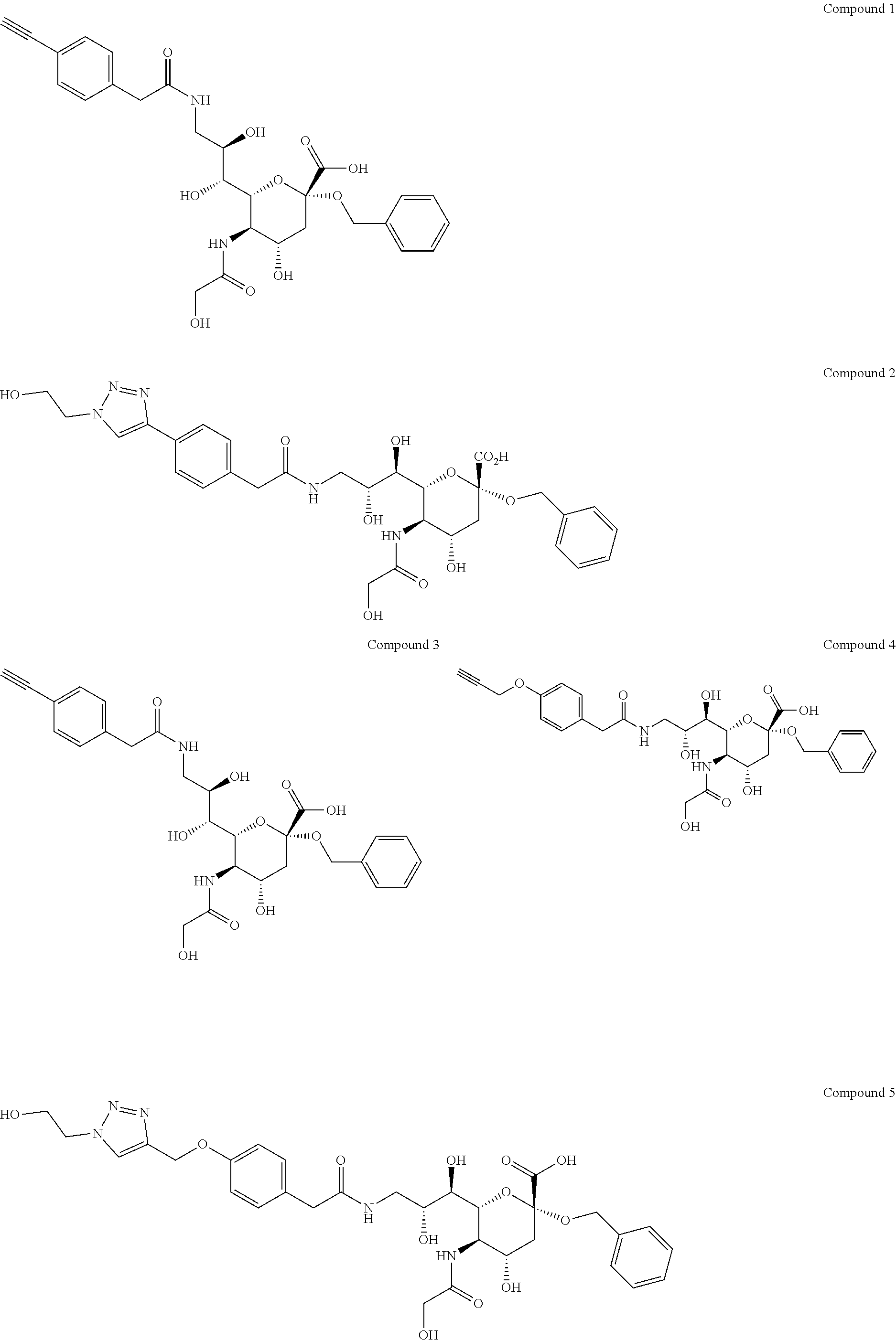

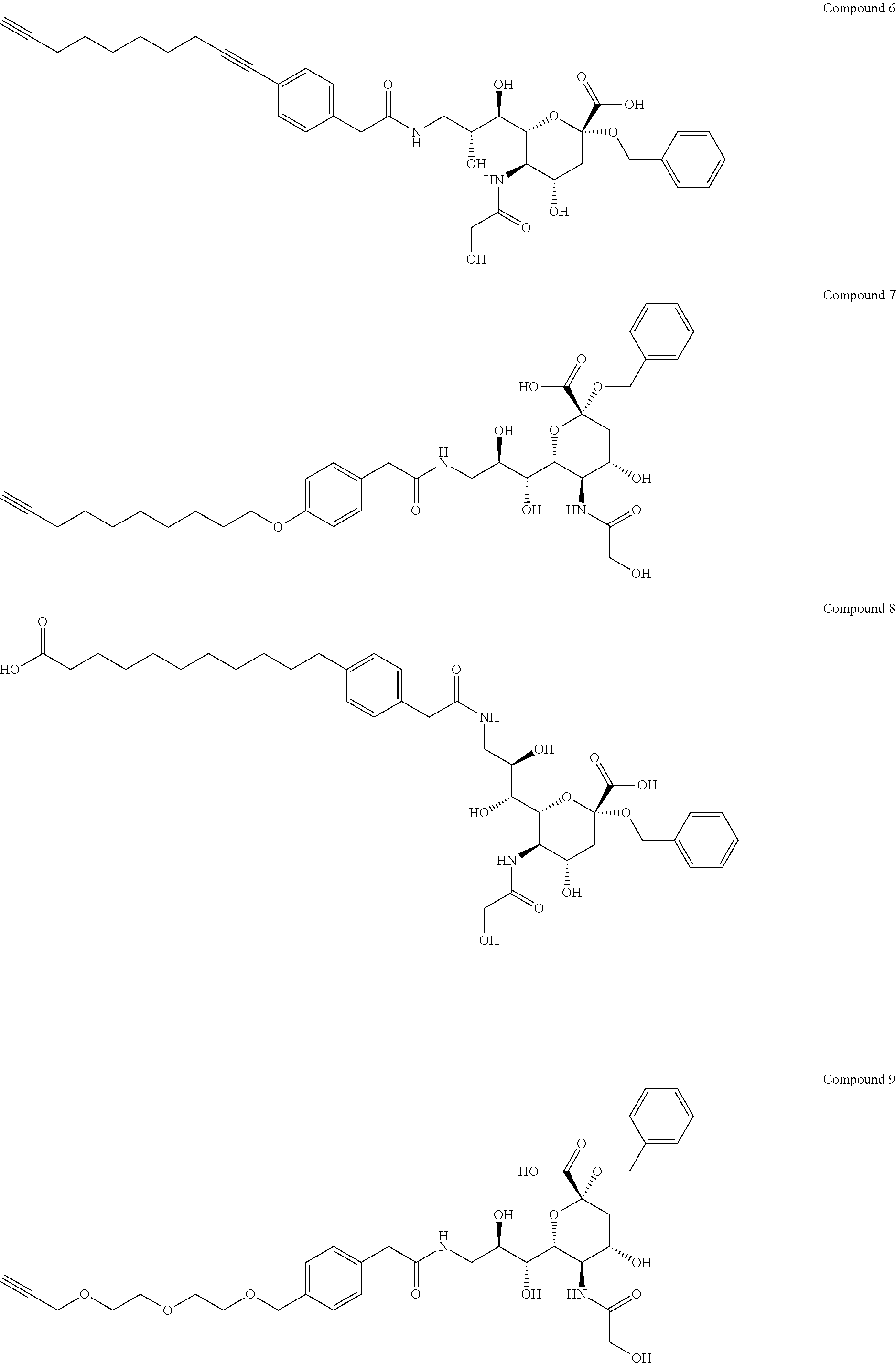 [SMILES: C#CCCCCCCC#Cc1ccc(CC(=O)NC[C@@H](O)[C@@H](O)[C@@H]2O[C@@](OCc3ccccc3)(C(=O)O)C[C@H](O)[C@H]2NC(=O)CO)cc1 ; C#CCCCCCCCCOc1ccc(CC(=O)NC[C@@H](O)[C@@H](O)[C@@H]2O[C@@](OCc3ccccc3)(C(=O)O)C[C@H](O)[C@H]2NC(=O)CO)cc1 ; O=C(O)CCCCCCCCCCc1ccc(CC(=O)NC[C@@H](O)[C@@H](O)[C@@H]2O[C@@](OCc3ccccc3)(C(=O)O)C[C@H](O)[C@H]2NC(=O)CO)cc1 ; C#CCOCCOCCOCc1ccc(CC(=O)NC[C@@H](O)[C@@H](O)[C@@H]2O[C@@](OCc3ccccc3)(C(=O)O)C[C@H](O)[C@H]2NC(=O)CO)cc1]

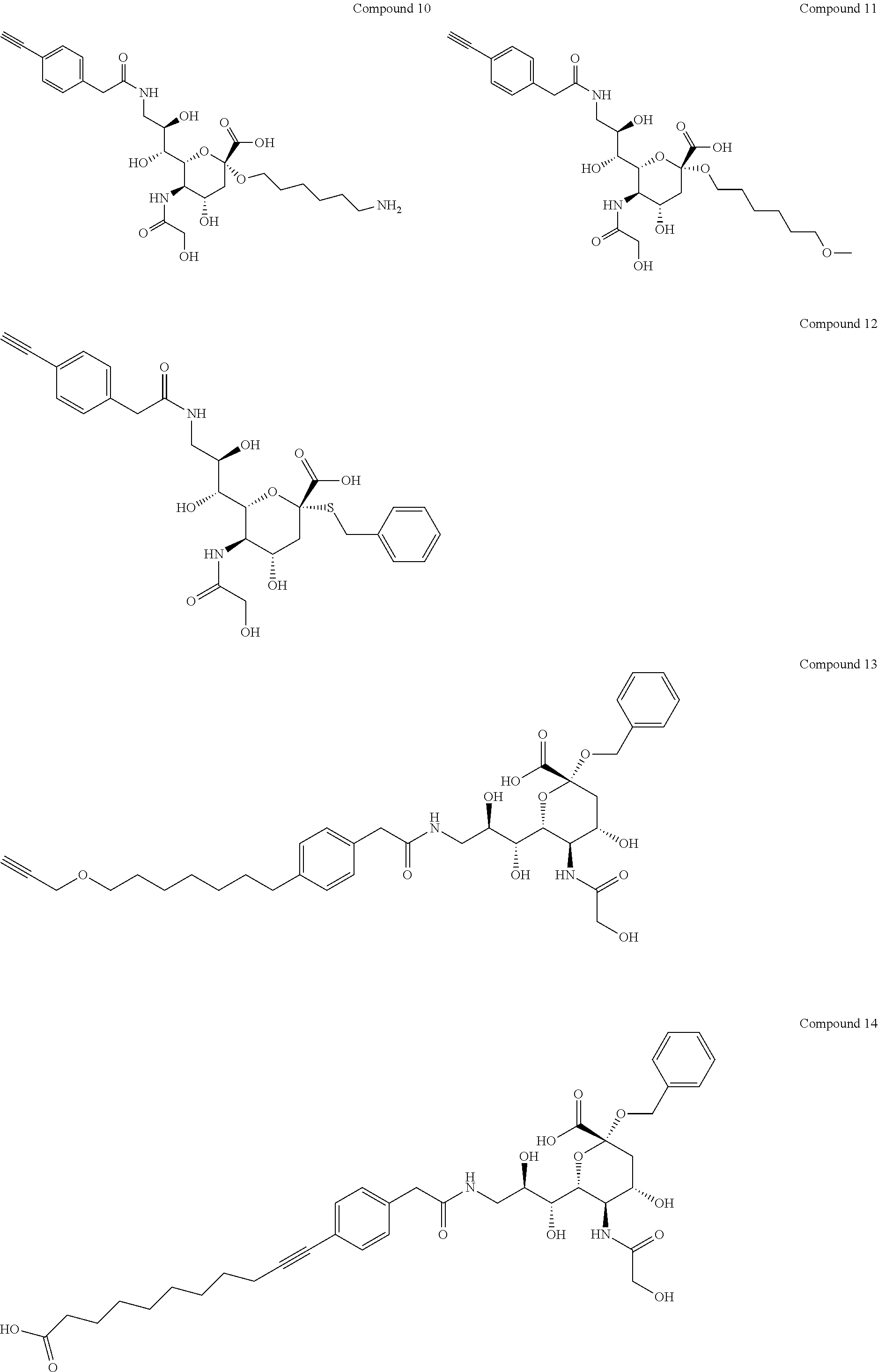

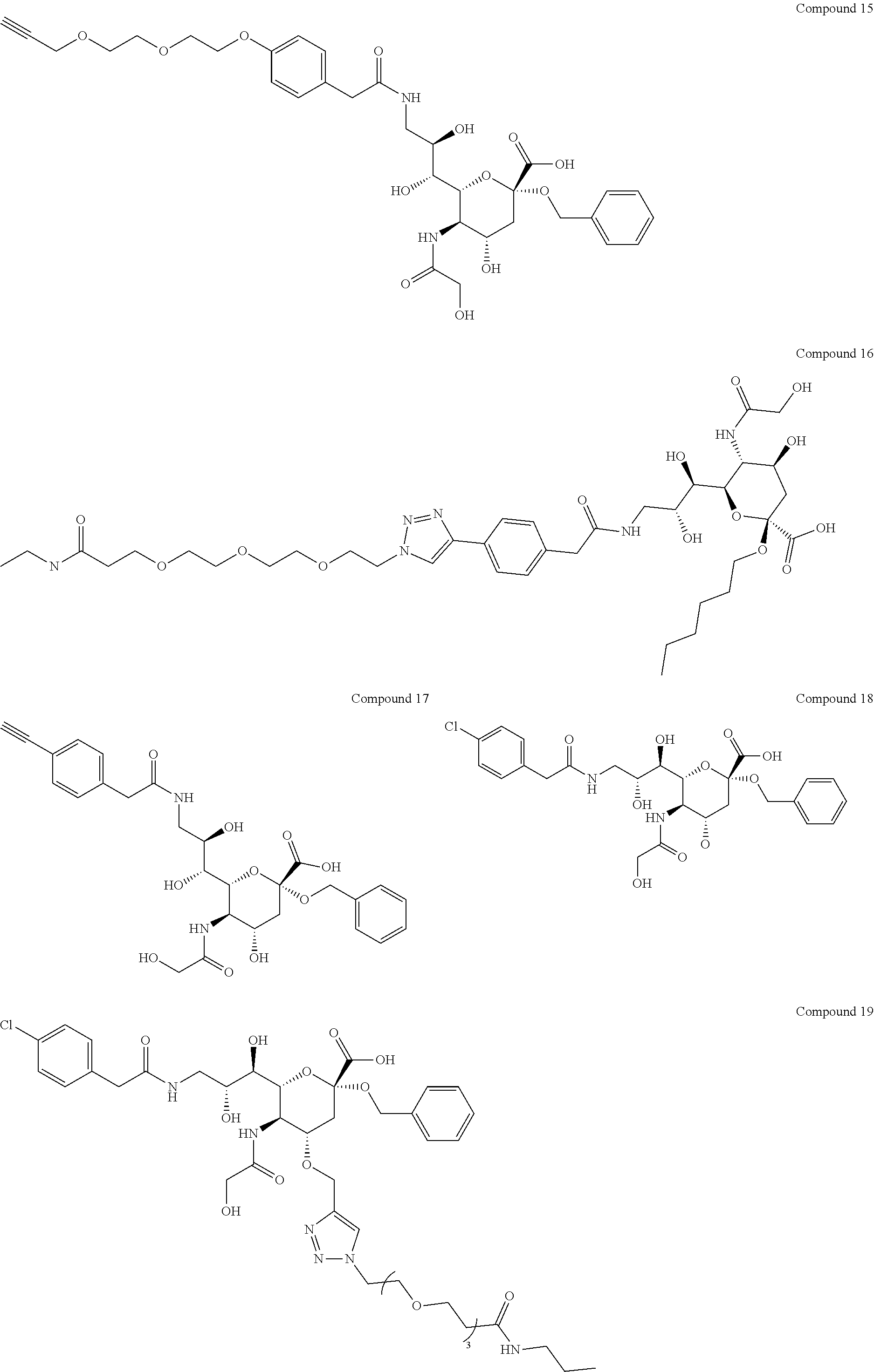

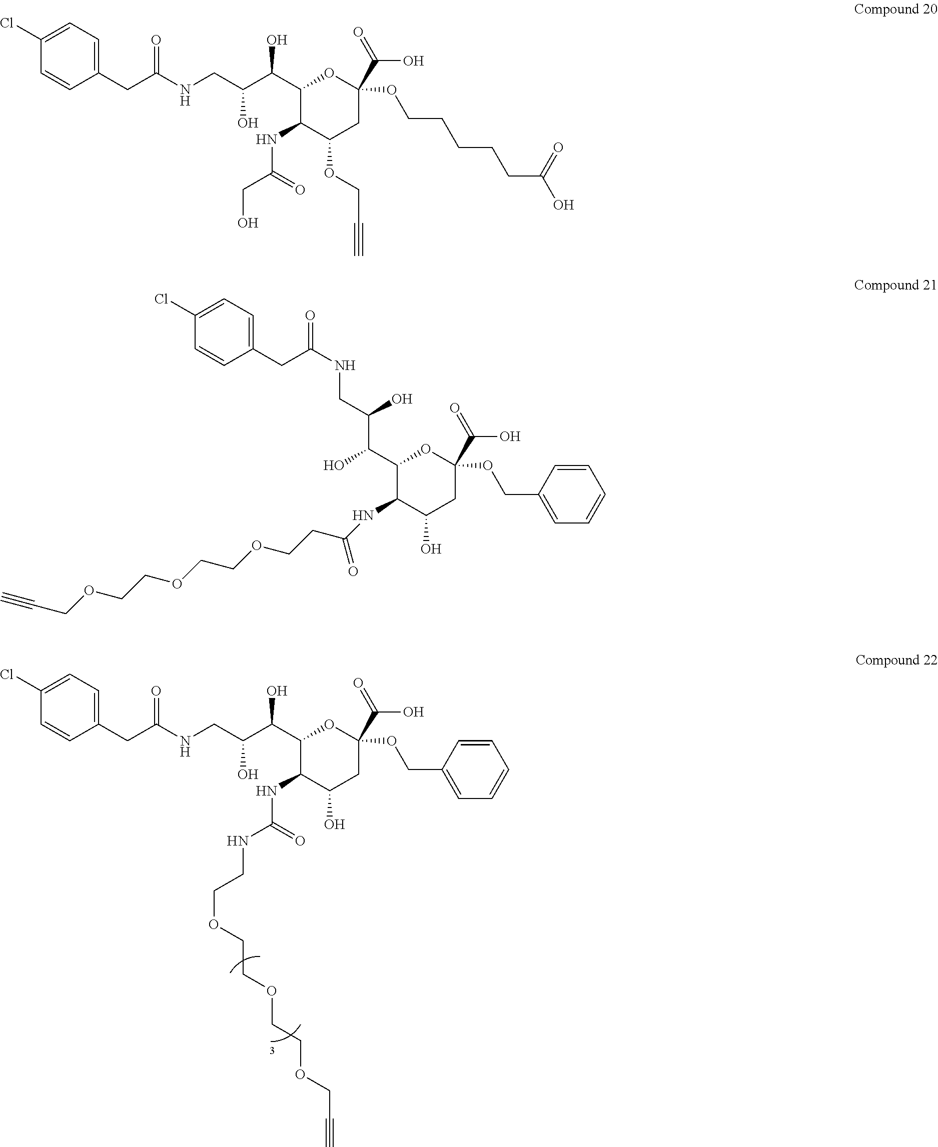

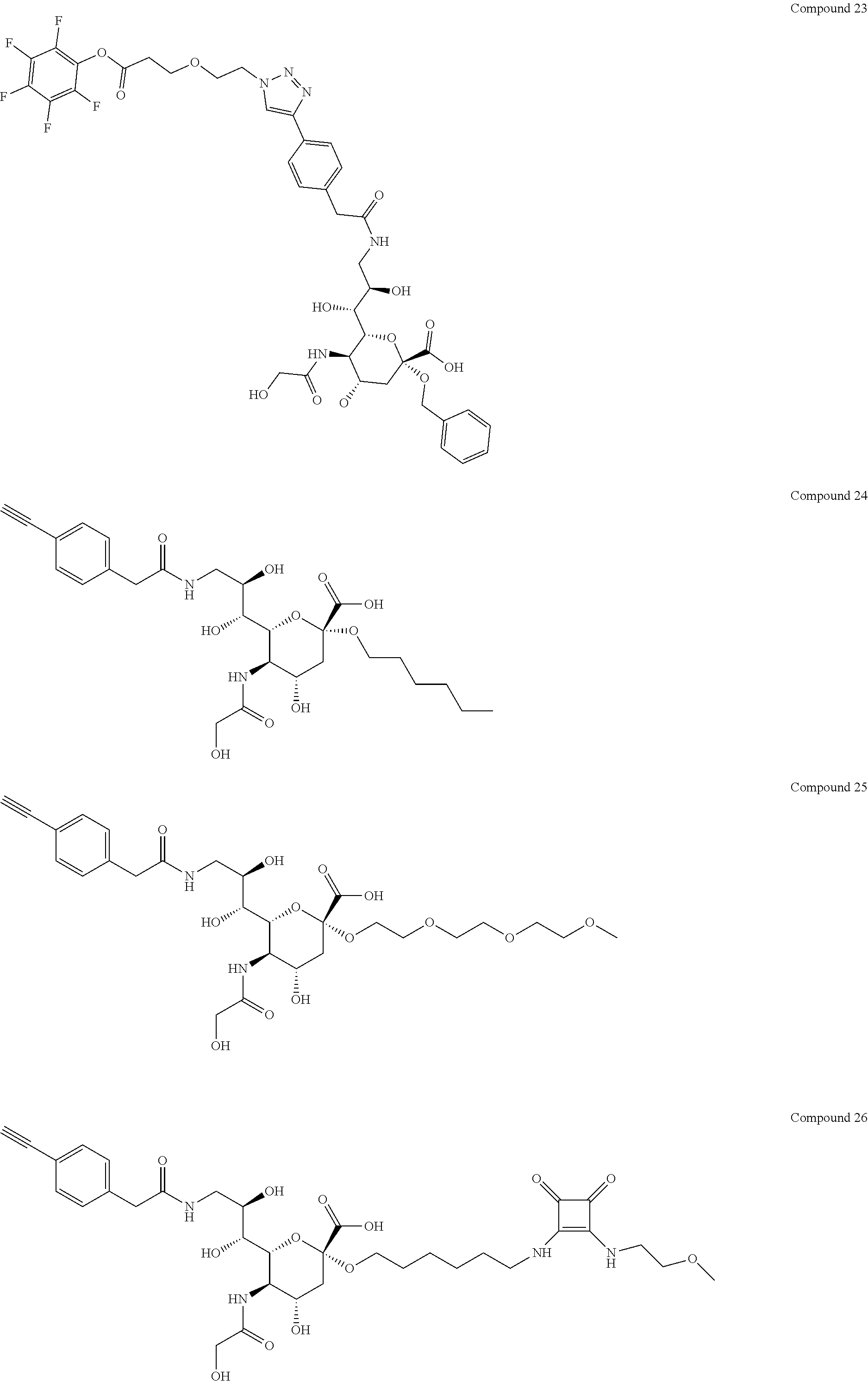

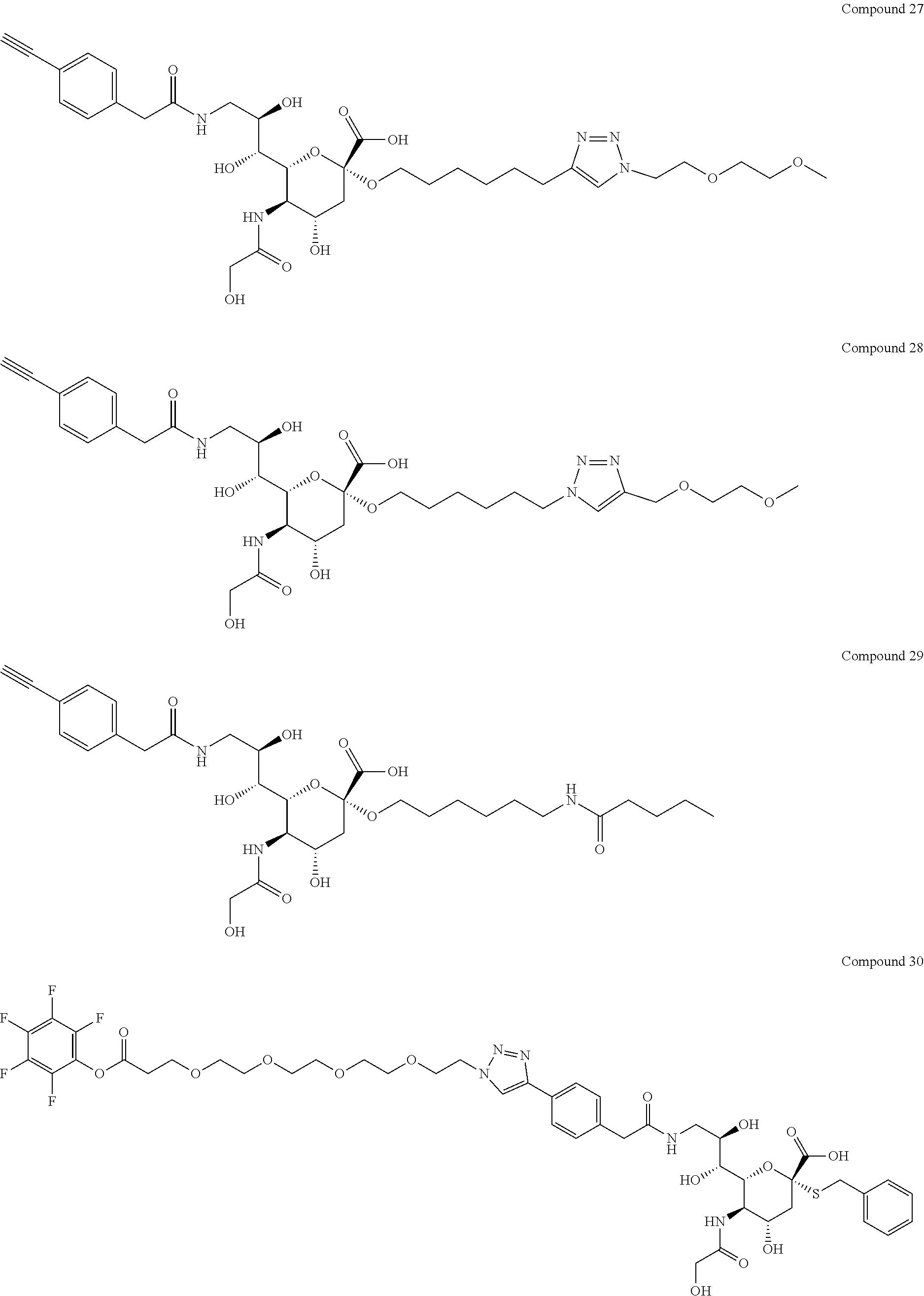

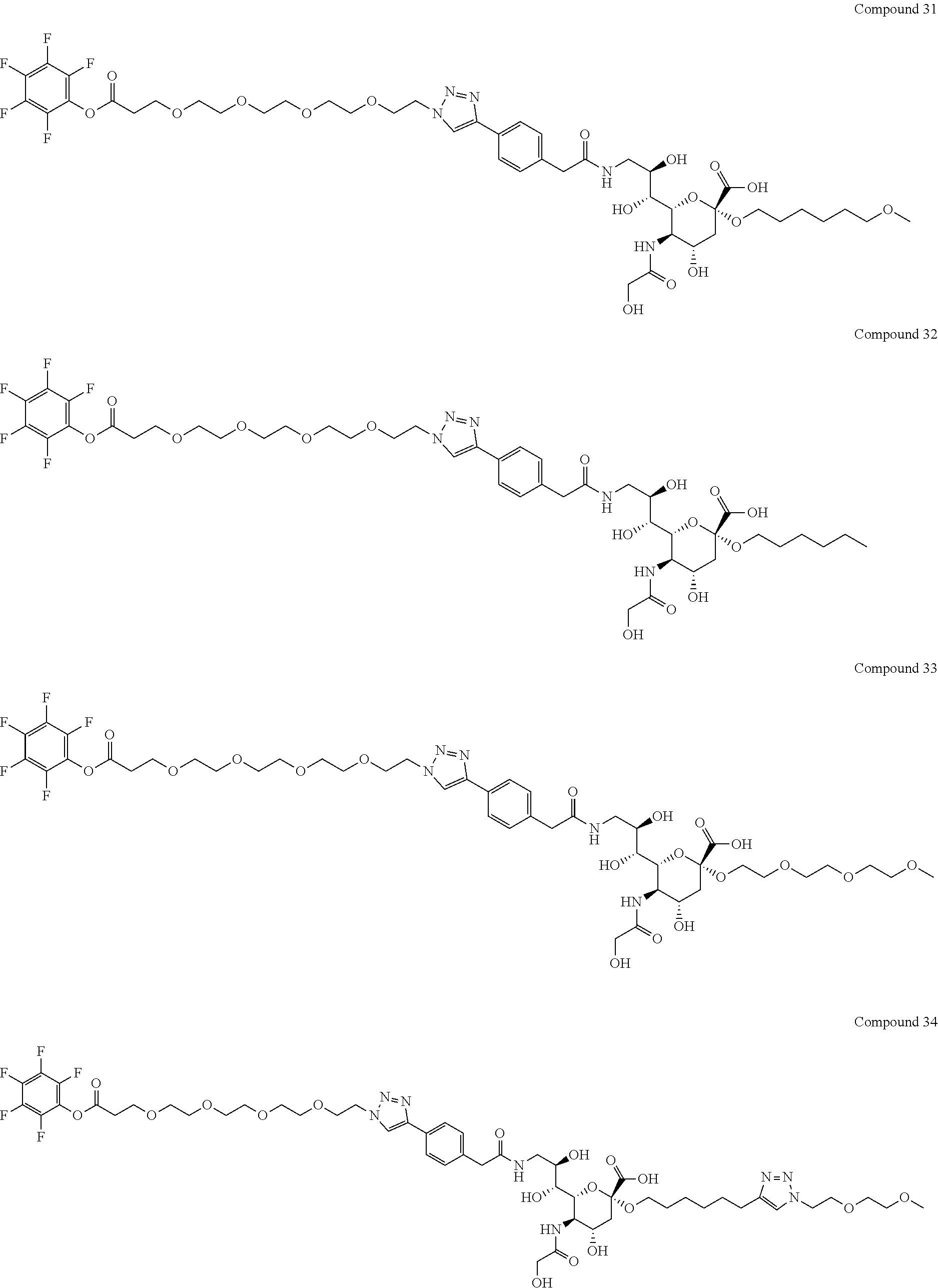

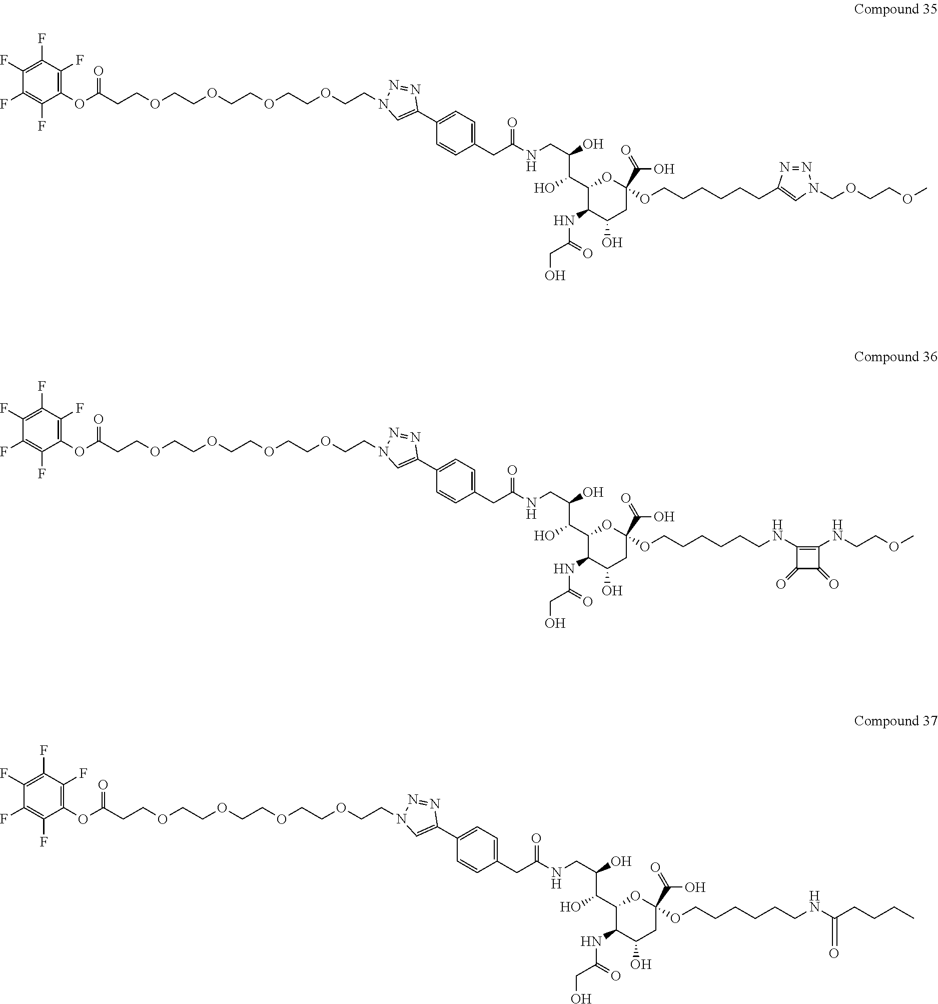 [SMILES: COCCOCn1cc(CCCCCCO[C@]2(C(=O)O)C[C@H](O)[C@@H](NC(=O)CO)[C@H]([C@H](O)[C@H](O)CNC(=O)Cc3ccc(-c4cn(CCOCCOCCOCCOCCC(=O)Oc5c(F)c(F)c(F)c(F)c5F)nn4)cc3)O2)nn1 ; COCCNc1c(NCCCCCCO[C@]2(C(=O)O)C[C@H](O)[C@@H](NC(=O)CO)[C@H]([C@H](O)[C@H](O)CNC(=O)Cc3ccc(-c4cn(CCOCCOCCOCCOCCC(=O)Oc5c(F)c(F)c(F)c(F)c5F)nn4)cc3)O2)c(=O)c1=O ; CCCCC(=O)NCCCCCCO[C@]1(C(=O)O)C[C@H](O)[C@@H](NC(=O)CO)[C@H]([C@H](O)[C@H](O)CNC(=O)Cc2ccc(-c3cn(CCOCCOCCOCCOCCC(=O)Oc4c(F)c(F)c(F)c(F)c4F)nn3)cc2)O1]

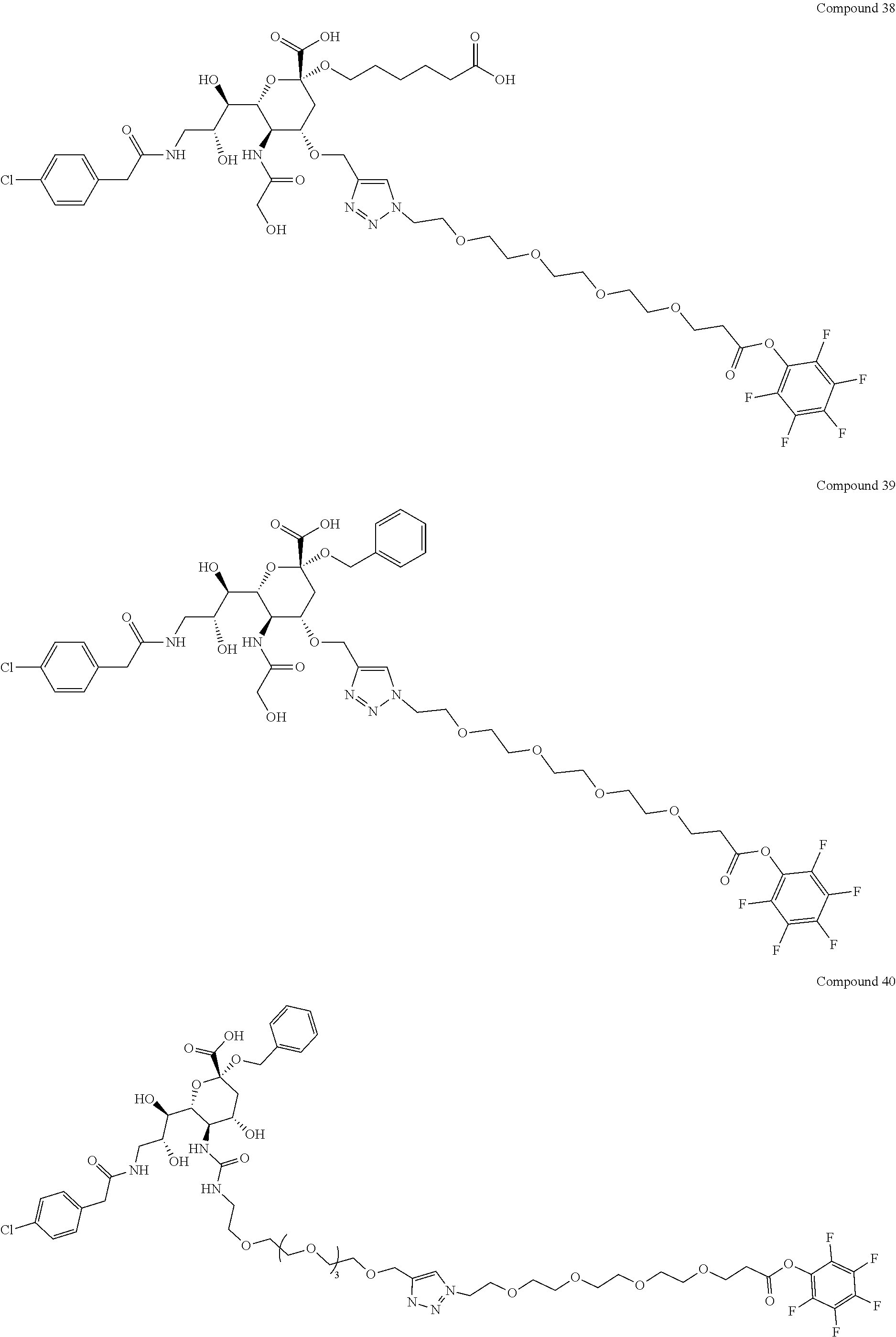

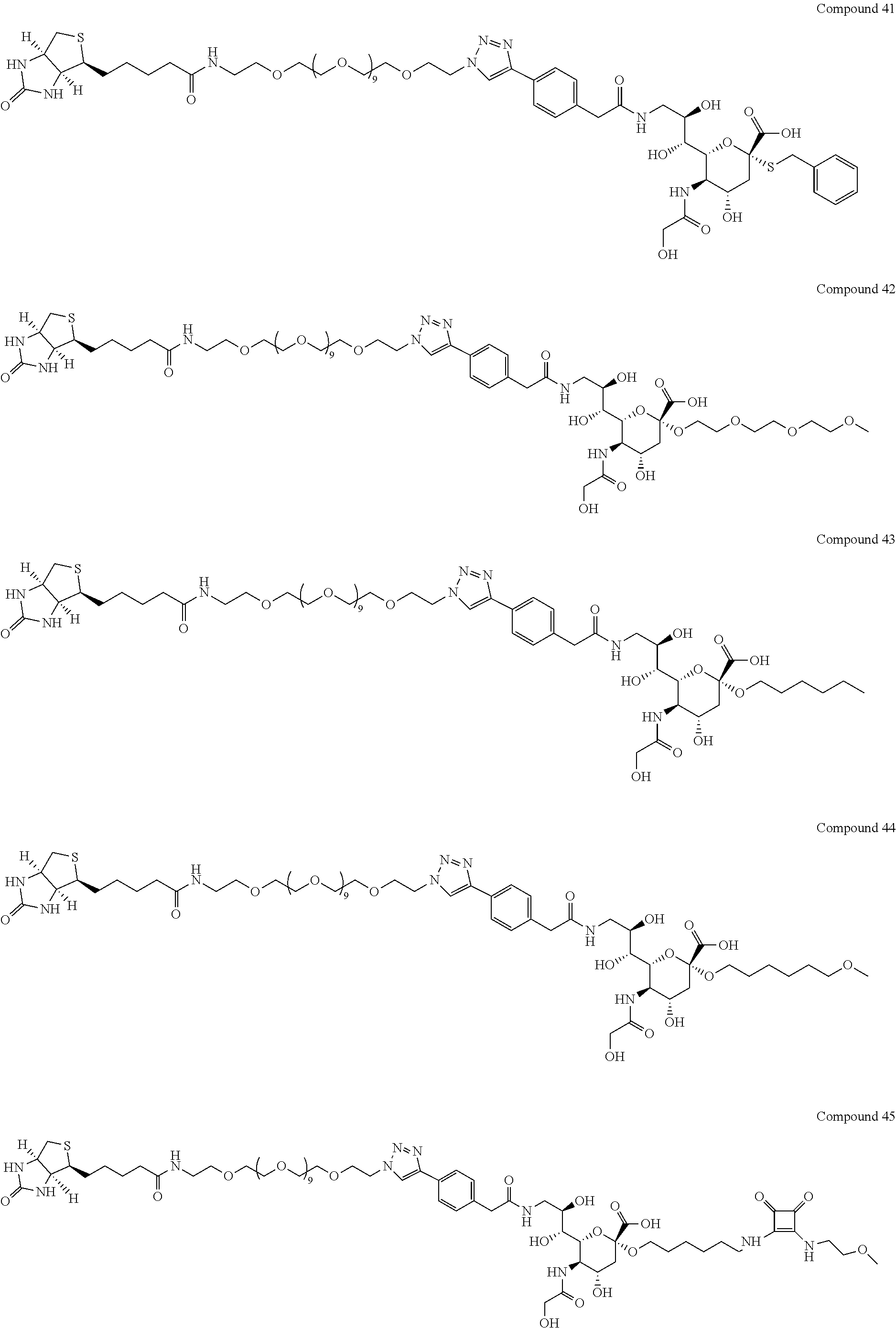

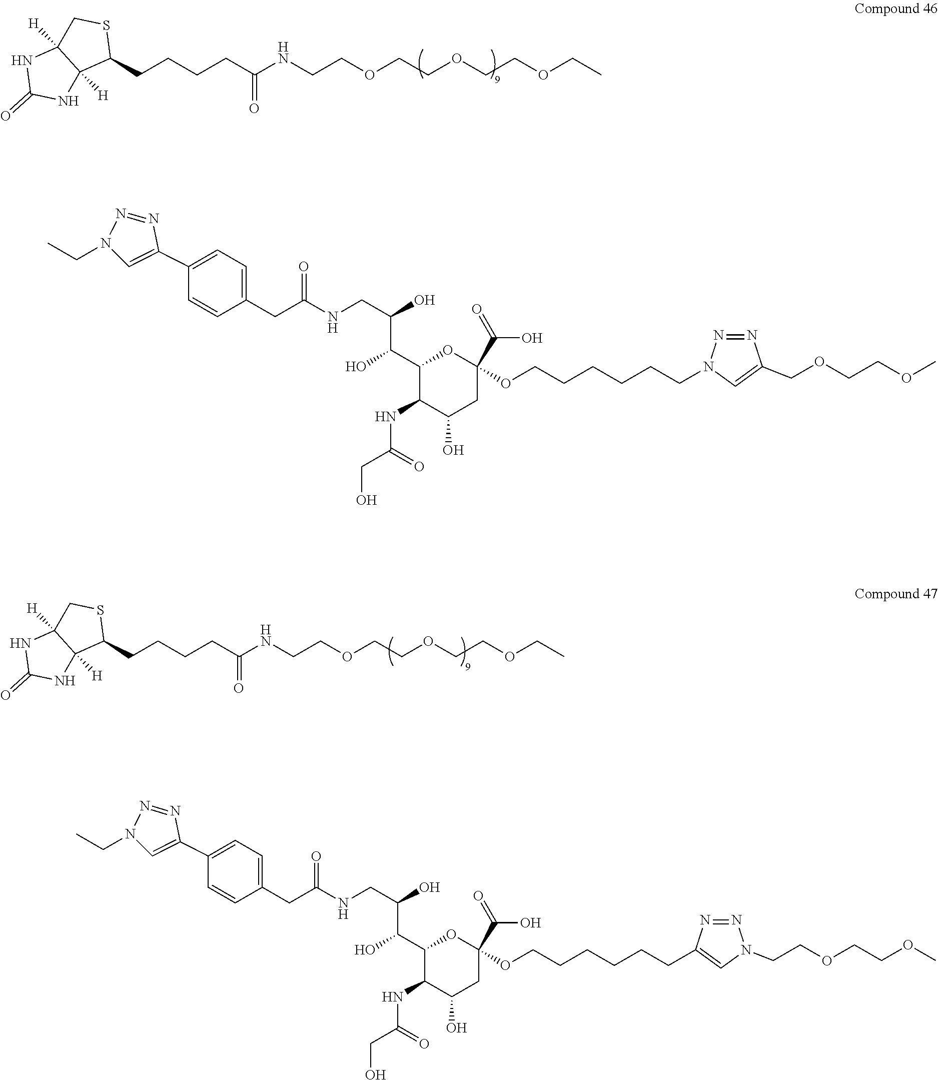

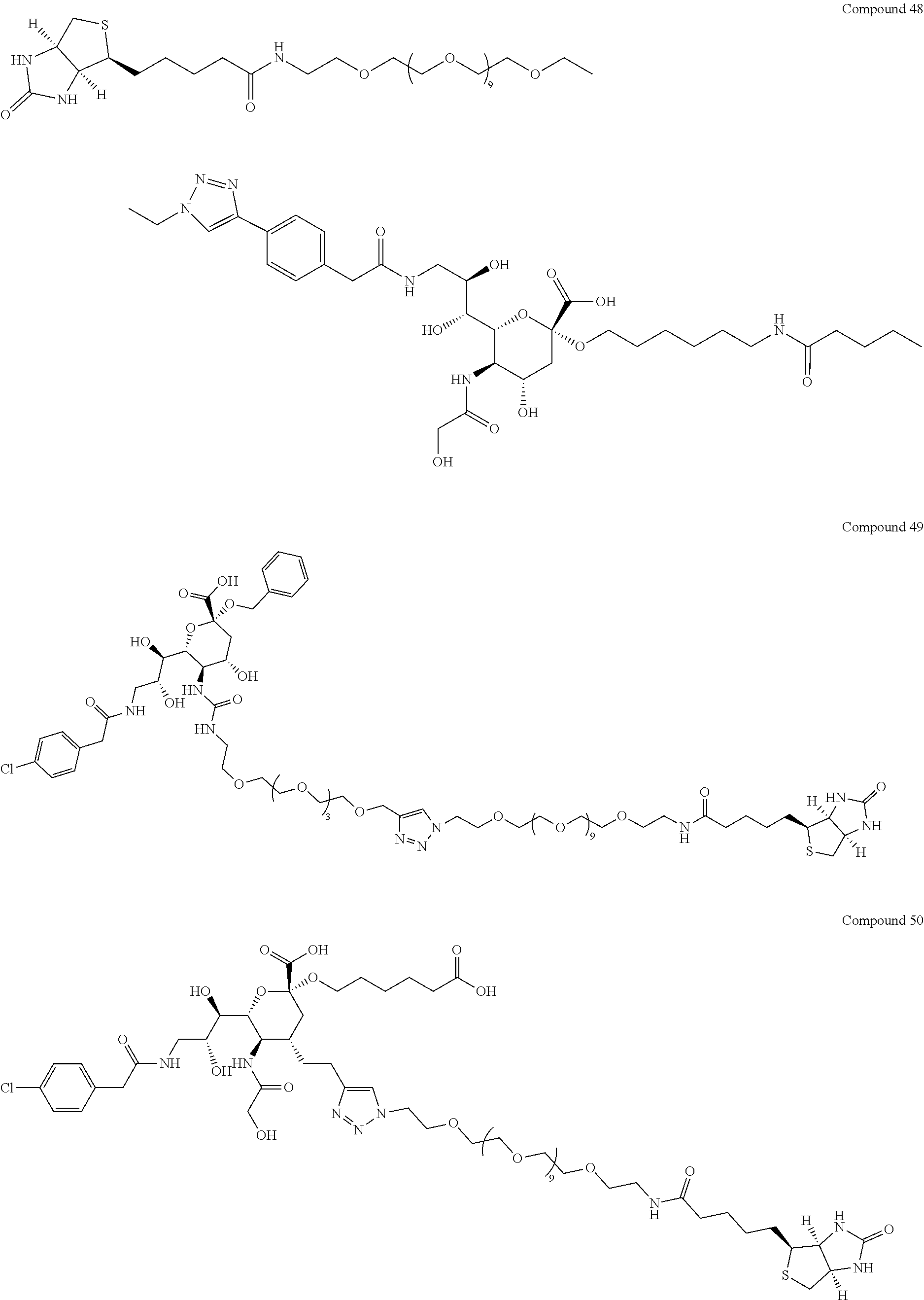

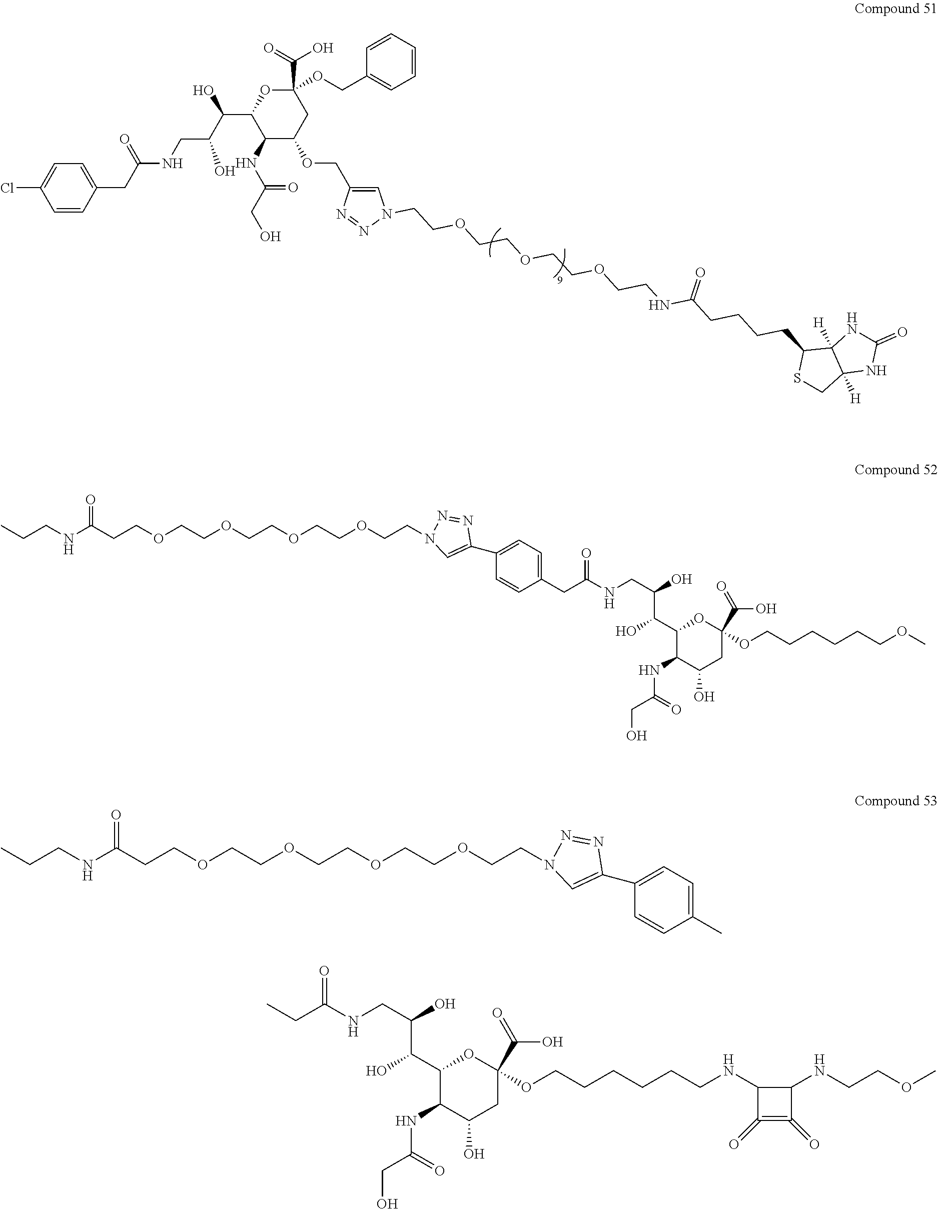

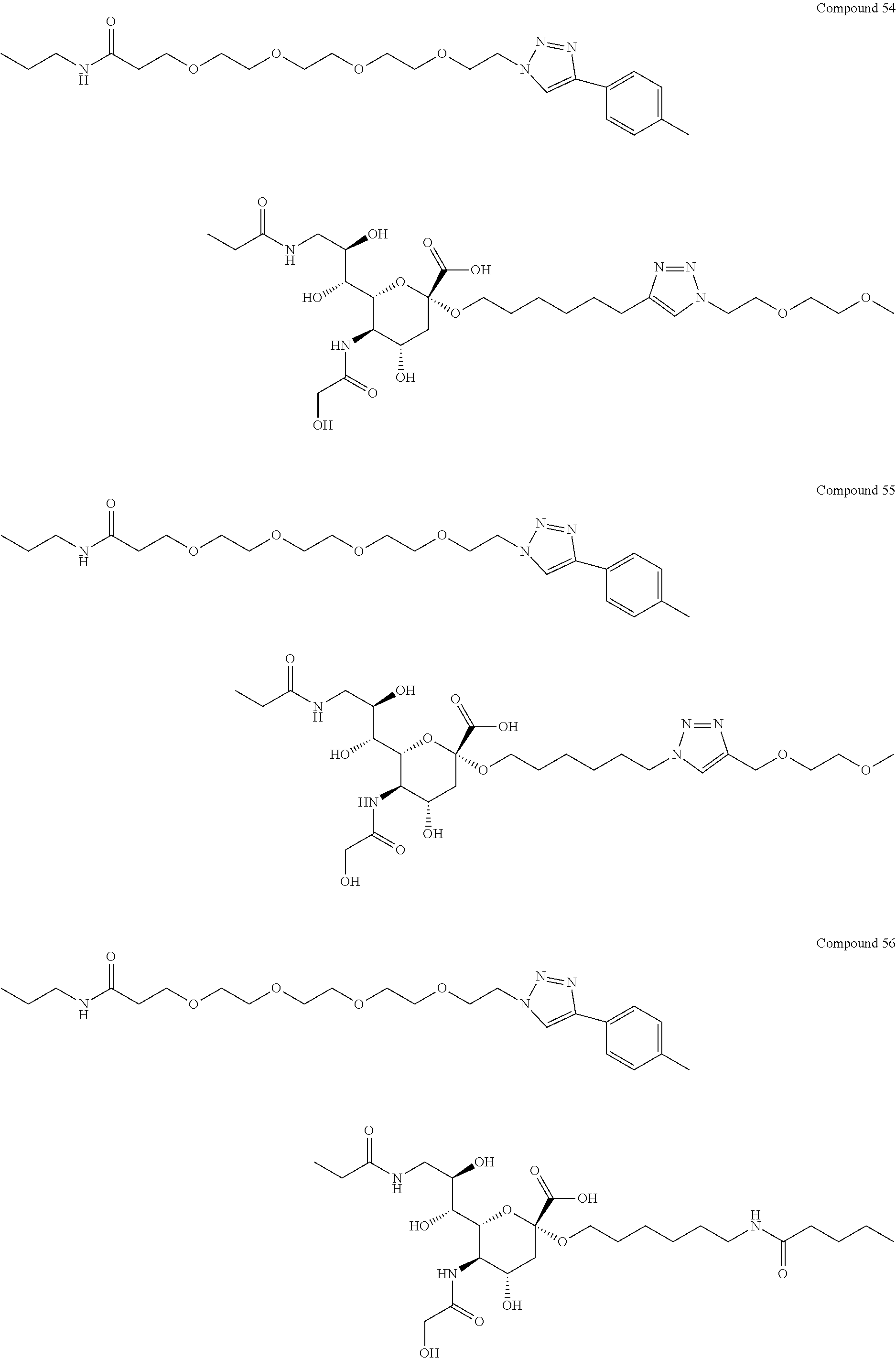

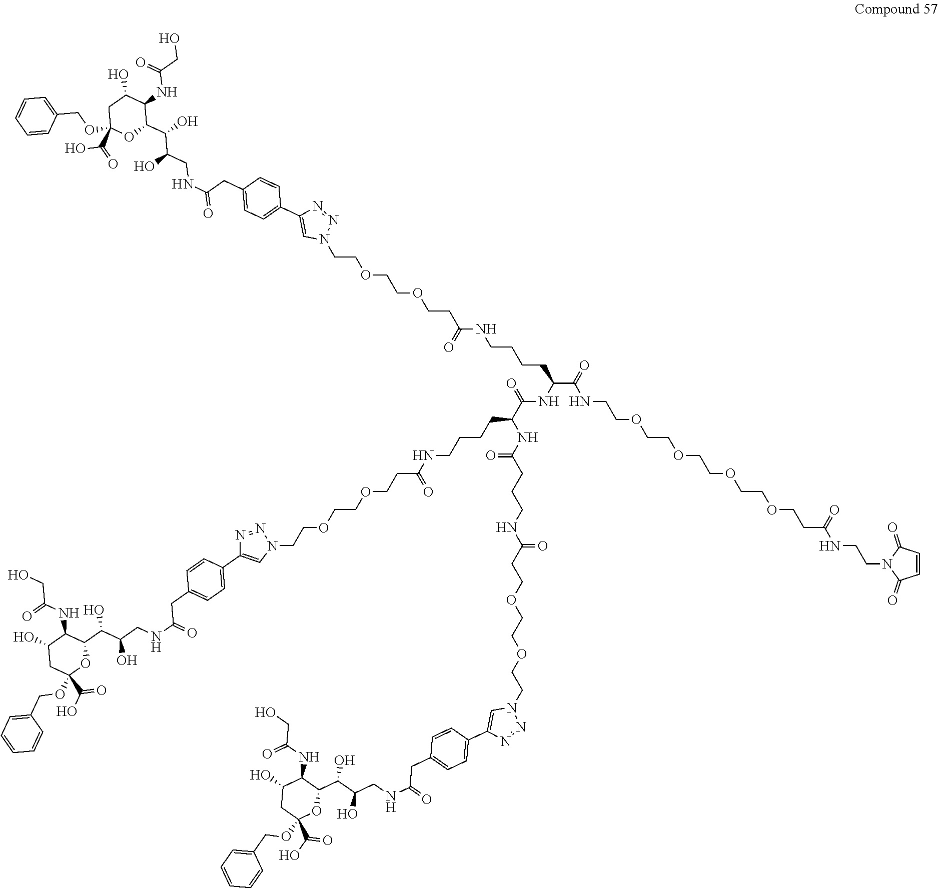 [SMILES: O=C(CCOCCOCCOCCOCCNC(=O)[C@H](CCCCNC(=O)CCOCCOCCn1cc(-c2ccc(CC(=O)NC[C@@H](O)[C@@H](O)[C@@H]3O[C@@](OCc4ccccc4)(C(=O)O)C[C@H](O)[C@H]3NC(=O)CO)cc2)nn1)NC(=O)[C@H](CCCCNC(=O)CCOCCOCCn1cc(-c2ccc(CC(=O)NC[C@@H](O)[C@@H](O)[C@@H]3O[C@@](OCc4ccccc4)(C(=O)O)C[C@H](O)[C@H]3NC(=O)CO)cc2)nn1)NC(=O)CCCNC(=O)CCOCCOCCn1cc(-c2ccc(CC(=O)NC[C@@H](O)[C@@H](O)[C@@H]3O[C@@](OCc4ccccc4)(C(=O)O)C[C@H](O)[C@H]3NC(=O)CO)cc2)nn1)NCCN1C(=O)C=CC1=O]

### Siglec Conjugates of Formula (Ia)

Provided are siglec conjugates that include a siglec ligand of formula (I) as described above and a biologically active substance (BAS). Provided by the present disclosure are siglec conjugates of formula (Ia):

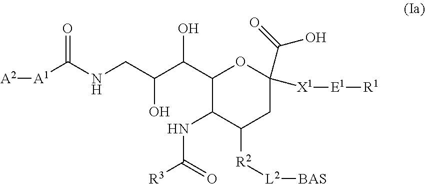

wherein:
A¹ is absent or —(CH₂)ₙ—, wherein n is an integer ranging from 1 to 10;
A² is alkyl, aryl, heteroaryl, cycloalkyl, or a substituted version thereof;
R¹ is H, alkyl, alkoxy, carboxyl, ester, amine, aryl, heteroaryl, cycloalkyl, heterocycle, carbohydrate, phosphate, squaramide, or a substituted version thereof;
R² is O, S, —NR⁴ R⁵, or —CR⁴ R⁵; wherein R⁴ and R⁵ are each independently selected from the group consisting of H, alkyl, substituted alkyl, alkenyl, substituted alkenyl, alkynyl, substituted alkynyl, cycloalkyl, substituted cycloalkyl, heterocyclyl, substituted heterocyclyl, aryl, substituted aryl, heteroaryl, and substituted heteroaryl;
R³ is OR^{3′}, NHR^{3′}, alkyl, —CH₂OR^{3′}, —CH₂NHR^{3′}, wherein R^{3′} is H, alkyl, aryl, heteroaryl, cycloalkyl, or a substituted version thereof;
X¹ is O, S, —CH₂—, —CH(OH)—, or triazole;
E¹ is absent, —(CH₂CH₂O)_{g} or —(CH₂)_{g}—, wherein g is an integer ranging from 1 to 10;
L² is a linker; and
BAS is a biologically active substance;
a salt thereof, or a stereoisomer thereof.

In some cases, A² is alkyl or substituted alkyl. For instance, A² has the formula CR⁶R⁷R⁸, wherein R⁶, R⁷, and R⁸ are each independently selected from the group consisting of H, alkyl, substituted alkyl, alkenyl, substituted alkenyl, alkynyl, substituted alkynyl, cycloalkyl, substituted cycloalkyl, heterocyclyl, substituted heterocyclyl, aryl, substituted aryl, heteroaryl, and substituted heteroaryl.

In some cases, R¹ is alkyl or substituted alkyl. For instance, A² has the formula CR⁶R⁷R⁸, wherein R⁶, R⁷, and R⁸ are each independently selected from the group consisting of H, alkyl, substituted alkyl, alkenyl, substituted alkenyl, alkynyl, substituted alkynyl, cycloalkyl, substituted cycloalkyl, heterocyclyl, substituted heterocyclyl, aryl, substituted aryl, heteroaryl, and substituted heteroaryl.

In some cases, R³ is alkyl or substituted alkyl. For instance, A² has the formula CR⁶R⁷R⁸, wherein R⁶, R⁷, and R⁸ are each independently selected from the group consisting of H, alkyl, substituted alkyl, alkenyl, substituted alkenyl, alkynyl, substituted alkynyl, cycloalkyl, substituted cycloalkyl, heterocyclyl, substituted heterocyclyl, aryl, substituted aryl, heteroaryl, and substituted heteroaryl.

In some embodiments of formula (Ia), A¹ is —CH₂—.

In some embodiments of formula (Ia), A² is aryl, or a substituted version thereof.

In some embodiments of formula (Ia), X¹ is O.

In some embodiments of formula (Ia), E¹ is —CH₂—.

In some embodiments of formula (Ia), R¹ is phenyl.

In some embodiments of formula (Ia), R² is O.

In some embodiments of formula (Ia), R³ is —CH₂OH.

### Siglec Conjugates of Formula (Ha)

Provided are siglec conjugates that include a siglec ligand of formula (II) as described above and a biologically active substance (BAS). Provided by the present disclosure are siglec conjugates of formula (IIa):

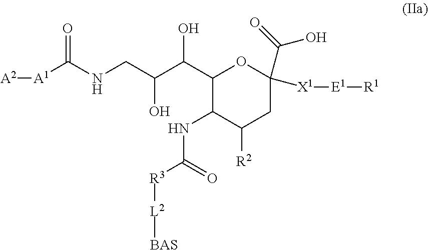

wherein:
A¹ is absent or —(CH₂)ₙ—, wherein n is an integer ranging from 1 to 10;
A² is alkyl, aryl, heteroaryl, cycloalkyl, or a substituted version thereof;
R¹ is H, alkyl, alkoxy, carboxyl, ester, amine, aryl, heteroaryl, cycloalkyl, heterocycle, carbohydrate, phosphate, squaramide, or a substituted version thereof;
R² is OH;
R³ is —O—, —NH—, —CH₂—, —CH₂O—, or —CH₂NH—;
X¹ is O, S, —CH₂—, —CH(OH)—, or triazole;
E¹ is absent, —(CH₂CH₂O)_{g}— or —(CH₂)_{g}—, wherein g is an integer ranging from 1 to 10;
L² is a linker; and
BAS is a biologically active substance;
a salt thereof, or a stereoisomer thereof.

In some cases, A² is alkyl or substituted alkyl. For instance, A² has the formula CR⁶R⁷R⁸, wherein R⁶, R⁷, and R⁸ are each independently selected from the group consisting of H, alkyl, substituted alkyl, alkenyl, substituted alkenyl, alkynyl, substituted alkynyl, cycloalkyl, substituted cycloalkyl, heterocyclyl, substituted heterocyclyl, aryl, substituted aryl, heteroaryl, and substituted heteroaryl.

In some cases, R¹ is alkyl or substituted alkyl. For instance, A² has the formula CR⁶R⁷R⁸, wherein R⁶, R⁷, and R⁸ are each independently selected from the group consisting of H, alkyl, substituted alkyl, alkenyl, substituted alkenyl, alkynyl, substituted alkynyl, cycloalkyl, substituted cycloalkyl, heterocyclyl, substituted heterocyclyl, aryl, substituted aryl, heteroaryl, and substituted heteroaryl.

In some embodiments of formula (IIa), A¹ is —CH₂—.

In some embodiments of formula (IIa), A² is aryl, or a substituted version thereof.

In some embodiments of formula (IIa), X¹ is O.

In some embodiments of formula (IIa), E¹ is —CH₂—.

In some embodiments of formula (IIa), R¹ is phenyl.

In some embodiments of formula (IIa), R² is OH.

In some embodiments of formula (IIa), R³ is —CH₂O—.

### Siglec Conjugates of Formula (IIa)

Provided are siglec conjugates that include a siglec ligand of formula (III) as described above and a biologically active substance (BAS). Provided by the present disclosure are siglec conjugates of formula (IIIa):

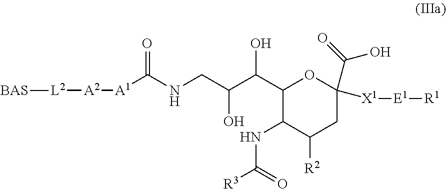

wherein:
A¹ is absent or —(CH₂)ₙ—, wherein n is an integer ranging from 1 to 10;
A² is alkyl, aryl, heteroaryl, cycloalkyl, or a substituted version thereof;
R¹ is H, alkyl, alkoxy, carboxyl, ester, amine, aryl, heteroaryl, cycloalkyl, heterocycle, carbohydrate, phosphate, squaramide, or a substituted version thereof;
R² is OH;
R³ is OR^{3′}, NHR^{3′}, alkyl, —CH₂OR^{3′}, —CH₂NHR^{3′}, wherein R^{3′} is H, alkyl, aryl, heteroaryl, cycloalkyl, or a substituted version thereof;
X¹ is O, S, —CH₂—, —CH(OH)—, or triazole;
E¹ is absent, —(CH₂CH₂O)_{g}— or —(CH₂)_{g}—, wherein g is an integer ranging from 1 to 10;
L² is a linker; and
BAS is a biologically active substance;
a salt thereof, or a stereoisomer thereof.

In some cases, A² is alkyl or substituted alkyl. For instance, A² has the formula CR⁶R⁷R⁸, wherein R⁶, R⁷, and R⁸ are each independently selected from the group consisting of H, alkyl, substituted alkyl, alkenyl, substituted alkenyl, alkynyl, substituted alkynyl, cycloalkyl, substituted cycloalkyl, heterocyclyl, substituted heterocyclyl, aryl, substituted aryl, heteroaryl, and substituted heteroaryl.

In some cases, R¹ is alkyl or substituted alkyl. For instance, A² has the formula CR⁶R⁷R⁸, wherein R⁶, R⁷, and R⁸ are each independently selected from the group consisting of H, alkyl, substituted alkyl, alkenyl, substituted alkenyl, alkynyl, substituted alkynyl, cycloalkyl, substituted cycloalkyl, heterocyclyl, substituted heterocyclyl, aryl, substituted aryl, heteroaryl, and substituted heteroaryl.

In some cases, R³ is alkyl or substituted alkyl. For instance, A² has the formula CR⁶R⁷R⁸, wherein R⁶, R⁷, and R⁸ are each independently selected from the group consisting of H, alkyl, substituted alkyl, alkenyl, substituted alkenyl, alkynyl, substituted alkynyl, cycloalkyl, substituted cycloalkyl, heterocyclyl, substituted heterocyclyl, aryl, substituted aryl, heteroaryl, and substituted heteroaryl.

In some embodiments of formula (IIIa), A¹ is —CH₂—.

In some embodiments of formula (IIIa), A² is alkyl, aryl, alkoxy, amine, or a substituted version thereof. In some embodiments of formula (IIIa), A² is aryl.

In some embodiments of formula (IIIa), X¹ is O. In some embodiments of formula (IIIa), X¹ is S.

In some embodiments of formula (IIIa), E¹ is absent. In some embodiments of formula (IIIa), E¹ is —CH₂—.

In some embodiments of formula (IIIa), R¹ is phenyl. In some embodiments of formula (IIIa), R¹ is substituted phenyl.

In some embodiments of formula (IIIa), R² is OH.

In some embodiments of formula (IIIa), R³ is —CH₂OH.

In some embodiments of formula (Ia), formula (IIa) and/or formula (IIIa), A¹ is absent.

In some cases, —(CH₂)ₙ—, wherein n is an integer ranging from 1 to 10, for example, A¹ is methyl, ethyl, propyl, butyl, pentyl, hexyl, heptyl, or octyl.

In some embodiments of formula (Ia), formula (IIa) and/or formula (IIIa), A² is alkyl, for example, saturated, straight-chain, or branched C₁-C₁₅ alkyl. In some cases, A² is methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1,1-dimethylpropyl, 1-ethylpropyl, hexyl, 1,1-dimethylbutyl, 2,2-dimethylbutyl, 3,3-dimethylbutyl, 1,2-dimethylbutyl, 1,3-dimethylbutyl, 2,3-dimethylbutyl, 1-methylpentyl, 2-methylpentyl, 3-methylpentyl, 4-methylpentyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, heptyl, or octyl.

In some cases, A² is a substituted version of alkyl, for example substituted with C₁-C₈ alkyl, halogen, amino, C₁-C₈ alkyloxy, cyano, nitro, aryl, heteroaryl, or C₃-C₈ cycloalkyl.

In some embodiments of formula (Ia), formula (IIa) and/or formula (IIIa), A² is an aryl, for example, C₆-C₁₄ aryl. In some cases, A² is phenyl, 1-naphthyl, 2-naphthyl, 1-biphenylene, or 2-biphenylene. In some cases, A² is a substituted version of an aryl, for example substituted with C₁-C₈ alkyl, halogen, hydroxy, amino, C₁-C₈ alkyloxy, C₁-C₈ haloalkyl, cyano, nitro, aryl, heteroaryl, or C₃-C₈ cycloalkyl.

In some embodiments of formula (Ia), formula (IIa) and/or formula (IIIa), A² is a heteroaryl for example, 5-10 membered heteroaryl containing one or more heteroatoms selected from S, N, O or SO₂. In some cases, A² is furyl, thienyl, pyrrolyl, pyrazolyl, oxazolyl, pyridinyl, pyrimidinyl, pyrazinyl or 4H-thieno[3,2-c]chromene. In some cases, A² is a substituted version of a heteroaryl, for example substituted with C₁-C₈ alkyl, halogen, amino, C₁-C₈ alkyloxy, cyano, nitro, aryl, heteroaryl, or C₃-C₈ cycloalkyl.

In some embodiments of formula (Ia), formula (IIa) and/or formula (IIIa), A² is a cycloalkyl, for example, C₃-C₈ cycloalkyl. In some cases, A² is cyclopropyl, cyclobutyl, cyclopentyl, cyclohexyl, cycloheptyl or cyclooctyl.

### Linkers

As discussed above, in the terminology of formula (I), (II) and (III), the “linker” referred to above is the L² moiety. For instance, the linker L² is represented by —(X²)ₘ—Z′, wherein each X² is independently selected from —(CH₂)ₕ—, —O—, —CH₂O—, —(CH₂CH₂O)ₕ—, —C(O)NH—, —C≡C—, —NH—C(O)—, squaramide, phosphamide, sulfonamide, and —CH₂C≡C—, triazole, carboxyl, ester, or substituted version thereof, wherein m is 0 or an integer ranging from 1 to 10, and h is an integer ranging from 1 to 10; and Z′ is polyethylene glycol, amino, alkoxy, amide, triazole, ester, aryl, heteroaryl, succinimidyl-thioether, or a substituted version thereof.

As discussed above, Z is a chemoselective functional group. For example, Z′ can be a group that is generated when a chemoselective functional group (Z) is reacted with a corresponding group to form a covalent bond. For example, if Z is an alkyne, then the Z group can be reacted with an azide group through an azide-alkyne Huisgen cycloaddition to form Z′ triazole group that connects the X² group to another group. As another example, Z can be an alkene group that can be reacted with a corresponding tetrazole group through a retro Diels-Alder reaction to form a covalent connection through a pyridazine Z′ group. For example, Z′ can comprise a polyethylene glycol (PEG) group. As such, in some cases the siglec ligand terminates with a Z chemoselective group that can selectively react and form a bond with another group. For instance, Z can react with another molecule of formula Z*—Z1, wherein Z and Z* can chemoselectively react to form Z, thereby giving a group of formula —Z′—Z1. As such, Z* is chemoselective partner group to Z, e.g. Z can be azide and Z* can be alkyne. This methodology allows for modular attachment and usage of chemoselective groups Z and Z*.

As such, the linker L² can have advantageous properties that improve the technical qualities of a conjugate.

Conjugates with Multiple Siglec Ligands

As discussed above, the present disclosure describes conjugates of siglec ligands of formula (I) or (II) or (III) and a biologically active substance. In some cases, these conjugates include a single siglec ligand.

In other cases, the conjugates include two or more siglec ligands and the biologically active substance. In such cases, the conjugates can be indirectly linked to the biologically active substance through a connector (C). Stated in another manner, each of the two or more siglec ligands can each be covalently bonded to the connector, and the connector can be covalently bonded to the biologically active substance. Likewise, any suitable number of siglec ligands can be connected to the biologically active substance through the connector (C), such as 3, 4, 5, 6, 7, 8, 9, 10, or more siglec ligands.

In some cases, siglec conjugates that include a siglec ligand of formula (I) as described above, linked to the biologically active substance (BAS) through a connector (C). Provided by the present disclosure are siglec conjugates of formula (Ib):
wherein:

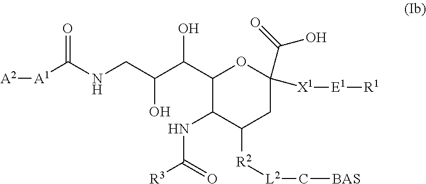

wherein:
A¹ is absent or —(CH₂)ₙ—, wherein n is an integer ranging from 1 to 10;
A² is alkyl, aryl, heteroaryl, cycloalkyl, or a substituted version thereof;
R¹ is H, alkyl, alkoxy, carboxyl, ester, amine, aryl, heteroaryl, cycloalkyl, heterocycle, carbohydrate, phosphate, squaramide, or a substituted version thereof;
R² is O, S, —NR⁴ R⁵, or —CR⁴ R⁵; wherein R⁴ and R⁵ are each independently selected from the group consisting of H, alkyl, substituted alkyl, alkenyl, substituted alkenyl, alkynyl, substituted alkynyl, cycloalkyl, substituted cycloalkyl, heterocyclyl, substituted heterocyclyl, aryl, substituted aryl, heteroaryl, and substituted heteroaryl;
R³ is OR^{3′}, NHR^{3′}, alkyl, —CH₂OR^{3′}, —CH₂NHR^{3′}, wherein R^{3′} is H, alkyl, aryl, heteroaryl, cycloalkyl, or a substituted version thereof;
X¹ is O, S, —CH₂—, —CH(OH)—, or triazole;
E¹ is absent, —(CH₂CH₂O)_{g}— or —(CH₂)_{g}—, wherein g is an integer ranging from 1 to 10;
L² is a linker;
C is a connector; and
BAS is a biologically active substance;
a salt thereof, or a stereoisomer thereof.

In some cases, siglec conjugates that include a siglec ligand of formula (II) as described above, linked to the biologically active substance (BAS) through a connector (C). Provided by the present disclosure are siglec conjugates of formula (IIb):

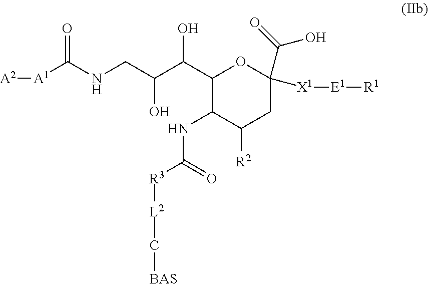

wherein:
A¹ is absent or —(CH₂)ₙ—, wherein n is an integer ranging from 1 to 10;
A² is alkyl, aryl, heteroaryl, cycloalkyl, or a substituted version thereof;
R¹ is H, alkyl, alkoxy, carboxyl, ester, amine, aryl, heteroaryl, cycloalkyl, heterocycle, carbohydrate, phosphate, squaramide, or a substituted version thereof;
R² is OH;
R³ is —O—, —NH—, —CH₂—, —CH₂O—, or —CH₂NH—;
X¹ is O, S, —CH₂—, —CH(OH)—, or triazole;
E¹ is absent, —(CH₂CH₂O)_{g}— or —(CH₂)_{g}—, wherein g is an integer ranging from 1 to 10;
L² is a linker;
C is a connector; and
BAS is a biologically active substance;
a salt thereof, or a stereoisomer thereof.

In some cases, siglec conjugates that include a siglec ligand of formula (III) as described above, linked to the biologically active substance (BAS) through a connector (C). Provided by the present disclosure are siglec conjugates of formula (IIIb):

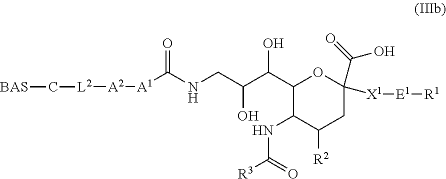

wherein:
A¹ is absent or —(CH₂)ₙ—, wherein n is an integer ranging from 1 to 10;
A² is alkyl, aryl, heteroaryl, cycloalkyl, or a substituted version thereof;
R¹ is H, alkyl, alkoxy, carboxyl, ester, amine, aryl, heteroaryl, cycloalkyl, heterocycle, carbohydrate, phosphate, squaramide, or a substituted version thereof;
R² is OH;
R³ is OR^{3′}, NHR^{3′}, alkyl, —CH₂OR^{3′}, —CH₂NHR^{3′}, wherein R^{3′} is H, alkyl, aryl, heteroaryl, cycloalkyl, or a substituted version thereof;
X¹ is O, S, —CH₂—, —CH(OH)—, or triazole;
E¹ is absent, —(CH₂CH₂O)_{g}— or —(CH₂)_{g}—, wherein g is an integer ranging from 1 to 10;
L² is a linker;
C is a connector; and
BAS is a biologically active substance;
a salt thereof, or a stereoisomer thereof.

Substituents A¹, A², R¹, R², R³, X¹, E¹, and L² in formula (Ib), formula (IIb) and/or formula (IIIb) are as described in the above paragraphs.

In some cases, a branching location of the connector includes an amino acid residue or a derivative thereof, e.g. lysine or a derivative thereof. Amino acid residues include amino acids commonly found in naturally occurring proteins (e.g., Ala or A, Cys or C, Asp or D, Glu or E, Phe or F, Gly or G, His or H, Ile or I, Lys or K, Leu or L, Met or M, Asn or N, Pro or P, Gln or Q, Arg or R, Ser or S, Thr or T, Val or V, Trp or W, Tyr or Y). In some embodiments, amino acid residues used in the connectors and connector subunits described herein also include amino acid analogs and amino acid derivatives, which are natural amino acids with modified side chains or backbones. Amino acid analogs also include amino acid analogs with the same stereochemistry as in the naturally occurring D-form, as well as the L-form of amino acid analogs. In some instances, the amino acid analogs share backbone structures, and/or the side chain structures of one or more natural amino acids, with difference(s) being one or more modified groups in the molecule. Such modification may include, but is not limited to, substitution of an atom (such as N) for a related atom (such as S), addition of a group (such as methyl, or hydroxyl, etc.) or an atom (such as Cl or Br, etc.), deletion of a group, substitution of a covalent bond (single bond for double bond, etc.), or attachment of another group to the side chain or backbone, or combinations thereof. For example, amino acid analogs may include α-hydroxy acids, and α-amino acids, and the like. In some instances, an amino acid analog or amino acid derivative can include another group, such as another sialic acid moiety (X), attached to the side chain or backbone of the amino acid analog or amino acid derivative through an optional connector.

For instance, the branching location of connector (C) can have the formula shown below, wherein each location marked with an asterisk (*) is a site for heading towards a siglec group or the biotherapeutic or autoantigen.

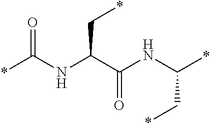 [SMILES: *C[C@@H](*)NC(=O)[C@H](C*)NC(*)=O]

In some cases, the branching location of the connector (C) does not comprise an aryl group or a heteroaryl group. In some cases, the branching location of the connector (C) comprises an alkyl group, an amide group, an amino acid residue group, or a combination thereof.

In some cases, C is absent. In such cases, the siglec ligand terminating with group L¹ is directly covalently bonded to the biologically active substance (BAS).

### Aspects of Siglec Ligands and Conjugates Thereof

As discussed above, the conjugates provided herein include a siglec ligand and biologically active substance (BAS). As used herein, the term “biologically active substance” refers to a substance that causes a change in a biological system when the biological system is contacted with the substance. In some cases, the biologically active substance is a protein. In some cases, the biologically active substance is a biotherapeutic. In some cases, the biologically active substance is an autoantigen.

### Biotherapeutic

The term “biotherapeutic” refers to a composition that is composed of sugars, amino acids, proteins, lipids or nucleic acids or complex combinations of these substances and that is therapeutic in an individual. Examples of biotherapeutics include protein therapeutics (e.g. antibodies, fusion proteins, and enzymes), viral therapeutics (e.g. viral particle), cell therapeutics, and nucleic acid therapeutics (e.g. polypeptides and nucleic acids).

Any protein or nucleic acid biotherapeutic may serve as the biotherapeutic that is engineered to become a hypoimmunogenic biotherapeutic according to the present disclosure, including, for example, a protein, e.g. an antibody, a fusion protein, an enzyme, a viral particle, a DNA molecule or an RNA molecule. The biotherapeutic may be naturally occurring, for example a naturally occurring protein that is delivered to a patient as a therapeutic, a naturally occurring capsid, etc. The biotherapeutic may be an engineered protein, for example, an antibody therapeutic, a fusion or “chimeric” protein, i.e. a protein comprising protein domains from two or more different proteins, or an entirely non-natural protein, i.e. having 30% identity or less with any naturally occurring protein across its functional domains (see e.g. Chen et al. (2020) De novo design of protein logic gates. Science 368 (6486): 78-84; and Polizzi et al. (2020) A defined structural unit enables de novo design of small-molecule-binding proteins Science 369 (6508): 1227-1233). In some embodiments, the biotherapeutic is a variant of a naturally occurring protein or a known engineered protein. By “variant” it is meant a mutant of a protein having less than 100% sequence identity with the protein from which it is derived. For example, a variant protein may be a protein having 60% sequence identity or more with a full length native protein, e.g. 65%, 70%, 75%, or 80% or more identity, such as 85%, 90%, or 95% or more identity, for example, 98% or 99% identity with the full length native protein. Variants also include fragments of naturally occurring proteins, particularly those having comparable or improved activity over the naturally occurring protein. The biotherapeutic may be derived from any source, e.g. human, non-human, or engineered.

Of particular interest is the suppression of an immune response to a biotherapeutic, and more particularly, of a B cell response to the biotherapeutic. Accordingly, in some embodiments, the siglec ligand is a ligand for a siglec that is expressed on B lymphocytes, for example siglec-2 (also called CD22), siglec-5 (CD170), siglec-6, siglec-9 (CD329), or siglec-10 (siglec-G). In some embodiments, the siglec is siglec-2. In some embodiments, the siglec is siglec-5. In some embodiments, the siglec is siglec-6. In some embodiments, the siglec is siglec-9. In some embodiments, the siglec is siglec-10. In some embodiments, the hypoimmunogenic biotherapeutic has been engineered to comprise the sialic acid ligands for one siglec. In other embodiments, the hypoimmunogenic biotherapeutic has been engineered to comprise the Siglec ligands for two or more siglecs, e.g. for 3 siglecs or for 4 siglecs, in certain cases, for 5 siglecs. In some cases there are 5, 6, 7, 8, 9, 10, 11, 12, 13, or 15 siglecs.

In some embodiments, the protein is an antibody or fragment thereof, for example a monoclonal antibody, a bispecific antibody, a trispecific antibody, an scFv, a Fab, a camelid nanobody, etc. Nonlimiting examples of antibodies for which the engineering contemplated herein finds particular use include adalimumab and infliximab (for the treatment of autoimmune or an inflammatory disease such as rheumatoid arthritis, psoriatic arthritis, ankylosing spondylitis, Crohn's disease, ulcerative colitis, psoriasis, hidradenitis suppurativa, uveitis, or juvenile idiopathic arthritis), cetuximab (for the treatment of cancers, including for example metastatic colorectal cancer, metastatic non-small cell lung cancer and head and neck cancer), natalizumab (for the treatment of multiple sclerosis), Lumoxiti/moxetumomab pasudotox (for the treatment of hairy cell leukemia), Tecentriq/atezolizumab (for the treatment of various cancers), Opdivo/Nivolumab (for the treatment of various cancers), Reopro/abciximab (anti-GPIIb/IIIa, for the prevention of thrombosis during and after coronary artery procedures such as angioplasty), Brentuximab (for the treatment of relapsed or refractory Hodgkin lymphoma (HL) and systemic anaplastic large cell lymphoma (ALCL)), Certolizumab pegol (for the treatment of Crohn's disease, rheumatoid arthritis, psoriatic arthritis and ankylosing spondylitis), Elotuzumab (for the treatment of relapsed multiple myeloma), Benralizumab (for the treatment of asthma), Vedolizumab (for the treatment of ulcerative colitis and Crohn's disease), Galcanezumab (for the treatment of migraines and cluster headaches), Rituximab (for the treatment of autoimmune diseases and various cancer), Alemtuzumab (for the treatment of chronic lymphocytic leukemia (CLL) and multiple sclerosis), Dupilumab (for the treatment of allergic diseases such as eczema (atopic dermatitis), asthma and nasal polyps), Golimumab (for the treatment of inflammation), Obinutuzumab (for the treatment of lymphomas, e.g. chronic lymphocytic leukemia, follicular lymphoma), Tildrakizumab (for the treatment of immunologically mediated inflammatory disorders), Erenumab (for the prevention of migraine), Mepolizumab (for the treatment of severe eosinophilic asthma, eosinophilic granulomatosis, and hypereosinophilic syndrome (HES)), Ramucirumab (for the treatment of solid tumors), Ranibizumab (for the treatment of “wet” age-related macular degeneration (AMD, also ARMD), diabetic retinopathy, and macular edema), Ustekinumab (for the treatment of psoriasis, Crohn's disease, and ulcerative colitis), Reslizumab (for the treatment of asthma), Ipilimumab (for the treatment of various cancers), Alirocumab (for the treatment of high cholesterol), Belimumab (for the treatment of systemic lupus erythematosus (SLE)), Panitumumab (for the treatment of various cancers), Avelumab (for the treatment of Merkel cell carcinoma, urothelial carcinoma, and renal cell carcinoma), Necitumumab (for the treatment of metastatic squamous non-small-cell lung carcinoma (NSCLC)), Mogamulizumab (for the treatment of relapsed or refractory mycosis fungoides and Sézary disease, relapsed or refractory CCR4+ adult T-cell leukemia/lymphoma (ATCLL), and relapsed or refractory CCR4+ cutaneous T cell lymphoma (CTCL)), Olaratumab (for the treatment of solid tumors), Brodalumab (for the treatment of inflammatory diseases), Eculizumab (for the treatment of paroxysmal nocturnal hemoglobinuria (PNH), atypical hemolytic uremic syndrome (aHUS), and neuromyelitis optica), Pertuzumab (for the treatment of metastatic HER2-positive breast cancer), Pembrolizumab (for the treatment of various cancers), and Tocilizumab (for the treatment of rheumatoid arthritis (RA) and systemic juvenile idiopathic arthritis).

In some embodiments, biotherapeutic is an antibody that is not an antibody that is specific for a receptor selected from a B cell receptor (BCR), a receptor for the Fc region of immunoglobulin E (FcİRI), a Toll Like receptor (TLR), a T-cell receptor (TCR), or complexes thereof.

As used herein, an antibody that specifically binds to a target antigen refers to an antibody comprising a complementarity determining region (CDR) domain that specifically recognizes and binds to the target antigen. Thus, an antibody that specifically binds to a B cell receptor or complex thereof refers to an antibody comprising a CDR that specifically recognizes and binds to a B cell receptor or a complex comprising a B cell receptor, an antibody that specifically binds to a receptor for the Fc region of IgE refers to an antibody comprising a CDR that specifically recognizes and binds to a receptor for the Fc region of IgE or a complex comprising a receptor for the Fc region of IgE, an antibody that specifically binds to a Toll like receptor refers to an antibody comprising a CDR that specifically recognizes and binds to a Toll-like receptor or a complex comprising a Toll-like receptor, and an antibody that specifically binds to a T-cell receptor refers to an antibody comprising a CDR that specifically recognizes and binds to a T-cell receptor or a complex comprising a T-cell receptor

In some embodiments, the protein is a native, or naturally occurring, protein. In other embodiments, the protein is an engineered protein. Examples of proteins for which the engineering contemplated herein finds particular use include erythropoietin (EPO, to stimulate the production of red blood cells), thrombopoietin (TPO, to stimulate the production of platelets), human growth hormone, tissue factor, IFNβ-1b (for the treatment of Multiple Sclerosis), IFNβ-1a (for the treatment of Multiple Sclerosis), IL-2 or the IL-2 mimetic aldesleukin (for the treatment of melanoma and renal cell carcinoma), exenatide (for the treatment of Type 2 Diabetes), albiglutide (for the treatment of Type 2 Diabetes), alefacept (to control inflammation in moderate to severe psoriasis with plaque formation, for the treatment of cutaneous T-cell lymphoma and T-cell non-Hodgkin lymphoma), palifermin (to stimulate the growth of cells that line the surface of the mouth and intestinal tract following chemotherapy), belatacept (to promote graft/transplant survival), and neutral and basic amino acid transport protein rBAT or b(0,+)-type amino acid transporter 1 (for the treatment of cystinuria).

In some embodiments, biotherapeutic is a protein that is not ovalbumin or immunoglobulin E (IgE).

In some embodiments, the protein is an enzyme, for example a metabolic enzyme, a lysosomal enzyme, a protease, a peptidase, etc. Nonlimiting examples of enzymes for which the engineering contemplated herein finds particular use include asparaginase from Erwinia chrysanthemi (for the treatment of leukemia), bacterial IdeS (for immunosuppression following tissue transplantation or in the administration of a therapy, e.g. a gene therapy, for which the patient had preexisting immunity; for treatment of IgG antibody-driven diseases, such as Systemic lupus erythematosus, Pemphigus vulgaris or IgA Nephropathy), bacterial mucinase (for the treatment of MUC+ cancers, e.g. MUC1+ cancers), Factor VIII (for the treatment of Hemophilia A), Factor IX (for the treatment of Hemophilia B), Factor Xa (to promote clotting), a complement degrading protease, e.g. from a pathogen such as a bacterial pathogen or fungal pathogen (e.g. Pseudomonas Elastase (PaE), Pseudomonas Alkaline protease (PaAP), Streptococcal pyrogenic Exotoxin B (SpeB), a gingipain from Porphyromonas gingivalis, Aspergillus Alkaline protease 1 (Alp1), C. albicans Secreted aspartyl proteinases 1 (Sap1), 2 (Sap2), and 3 (Sap3) for the treatment of complement-mediated disease, such as IgA nephropathy), phenylalanine ammonia-lyase or the mimetic pegvaliase (for the treatment of PKU), alpha-galactosidase A (for the treatment of Fabry Disease), acid α-glucosidase or the mimetic Alglucosidase alfa (GAA, for the treatment of Pompe Disease), glucocerebrosidase (GCase, for the treatment of Gaucher), aspartylglucosaminidase (AGA, for the treatment of Aspartylglucosaminuria), asfotase (for treatment of hypophosphatasia (HPP)), alpha-L-iduronidase (for the treatment of MPS I), iduronate sulfatase or the iduronate sulfatase mimetic idursulfase (for the treatment of MPS II), sulfaminase (for the treatment of MPS IIIa), α-N-acetylglucosaminidase (NAGLU, for the treatment of MPS IIIB), heparin acetyle CoA: α-glucosaminide N-acetyltransferase (HGSNAT, for the treatment of MPS IIIC), N-acetylglucosamine 6-sulfatase (GNS, for the treatment of MPS IIID), N-glucosamine 3-O-sulfatase (arylsulfatase G or ARSG, for the treatment of MPS IIIE), N-acetylgalactosamine 6-sulfatase(for the treatment of MPS IVA), beta-galactosidase (for the treatment of MPS IVB), N-acetylgalactosamine 4-sulfatase (for the treatment of MPS VI), beta-glucuronidase (for the treatment of MPS VI), palmitoyl protein thioesterase (PPT1, for the treatment of Batten disease/CLN1), Tripeptidyl peptidase (TPP1, for the treatment of Batten Disease/CLN2), arginase-1 or pegzilarginase (for the treatment of arginase-1 deficiency), or cystathionine beta synthase or Aeglea product AGLE-177 (for the treatment of cystathionine beta synthase (CBS) deficiency, also known as Classical Homocystinuria).

In some embodiments, biotherapeutic is an enzyme that is not Factor VIII.

In some embodiments, the protein is a viral protein or a viral particle, for example, a recombinant viral particle. By a “recombinant” virus or viral particle it is meant a virus/viral particle that comprises a genome comprising a polynucleotide that is heterologous to the virus, i.e., not found in nature to be associated with the capsid/envelope of the virus, wherein the polynucleotide encodes a gene product (RNA or protein). Recombinant viral particles find use in the delivery of polynucleotides that encode a therapeutic gene product for the purpose of gene therapy or oncolytic virus therapy. Gene therapy is a well-established art. Also well-established is the fact that gene therapy is severely hampered by the inability to readminister the same viral therapeutic more than once or a few times, owing to the fact that the viral particle will induce an immune response in an individual. As such, the ordinarily skilled artisan will appreciate that any viral particle used in gene therapy would benefit from engineering as contemplated herein.

Nonlimiting examples of viral particles that may serve as the biotherapeutic that is engineered to become a hypoimmunogenic biotherapeutic according to the present disclosure include recombinant adeno-associated virus (rAAV) particles, e.g. an rAAV particle comprising a capsid VP1 protein from the group consisting of an AAV1, AAV2, AAV3b, AAV4, AAV5, AAV6, AAV7, AAV8, AAV9, AAVrh10, AAV11, AAV12, or AAV13 VP1 protein or a variant or pseudotyped virus thereof; recombinant human adenovirus particles, e.g. an rHAdV particle comprising a capsid protein from rHAdV-A, rHAdV-B, rHAdV-C, rHAdV-D, rHAdV-E, rHAdV-F, or rHAdV-G or a variant thereof; recombinant Herpes Simplex Virus (rHSV) particles, e.g. a rHSV1 or rHSV2 or variant or pseudotyped virus thereof; recombinant papillomavirus (PV) particles; recombinant polyomavirus particles; recombinant vaccinia virus particles; a recombinant cytomegalovirus (CMV) particle; a recombinant baculovirus particle; a recombinant human papillomavirus (HPV) particle; or a recombinant retrovirus particle, e.g. a recombinant lentivirus, recombinant human immunodeficiency virus (HIV) particle, Simian immunodeficiency virus (SIV) particle, Feline immunodeficiency virus (FIV) particle, Puma lentivirus (PLV) particle, Equine infectious anemia virus (EIAV) particle, Bovine immunodeficiency virus (BIV) particle, Caprine arthritis encephalitis virus particle, gammaretrovirus particle, and murine leukemia virus (MLV) particle, or variant or pseudotyped virus thereof.

In some embodiments, biotherapeutic is not a toxin. Generally, toxins are compounds that are harmful to cells in generally non-specific manner, i.e. a toxin will cause a similar amount of harm to different cells, even if such cells are from significantly different categories. In contrast, selectively damaging compounds will harm certain cells to a significantly greater degree than the harm inflicted on other types of cells. For instance, the selectively damaging compound can cause harm based on a biochemical process that is common in a lung cell but rare in a kidney cell, whereas a toxin can cause harm based on a biochemical process common to both lung and kidney cells. In some cases, the harm is cell death. In some cases, the toxin is Pseudomonas exotoxin A. In some embodiments, biotherapeutic is not a B cell modulator. Y does not increase or decrease the immune action of a B cell. Examples of modulation of the B cell include differentiation of the B cell into a biotherapeutic-specific mature B cell, e.g. plasma cells or memory cells, preventing B cells from producing antigen-specific antibodies, preventing the upregulation of activation markers such as CD69, promoting a decrease in viability of a biotherapeutic-specific B cell population. In some embodiments, B cell activation is inhibited only for those B cells with a B cell receptor that recognizes Y (in contrast to the entire B cell population recognizing X).

### Autoantigen

The term “autoantigen” (abbreviated as “AutoAg”) refers to an endogenous molecule present in a subject which the subject's immune system does not recognize as an endogenous molecule (i.e., as a self-antigen) and thus mounts an immune response to the self-antigen. Such an immune response is referred to as an autoimmune response. The autoantigen is encoded by an endogenous gene present in a subject and may be a polypeptide, e.g., a soluble or membrane-localized polypeptide, a lipidated, glycosylated, or otherwise post-translationally modified polypeptide; a nucleic acid (e.g., DNA, RNA); or a complex thereof. In some embodiments, the autoantigen is any autoantigen that elicits a B-cell driven immune response in an individual, where the individual produces B cells that bind to the autoantigen via B-cell receptors (BCRs) and upon binding to the autoantigen differentiate into plasma cells that produce autoantibodies that bind to the autoantigen.

In contrast to a biotherapeutic, an autoantigen is a molecule that is endogenously produced by the subject, whereas a biotherapeutic is a molecule that the subject does not produce and is exogenously supplied to the person, e.g., as a polypeptide or a gene encoding the polypeptide. Thus, an immune response to a biotherapeutic is not considered an autoimmune response. Rather, an immune response to a biotherapeutic is a normal immune response. A biotherapeutic may be composed of sugars, amino acids, proteins, lipids or nucleic acids or complex combinations of these substances. Unlike an engineered autoantigen, a biotherapeutic is not intended as an autoimmune-suppressive mimic of a non-suppressive and endogenous disease-driving antigen. Nonlimiting examples of biotherapeutics include protein therapeutics, e.g., antibody therapeutics, fusion protein therapeutics, enzyme therapeutics, viral therapeutics, cell therapeutics, and nucleic acid therapeutics.

Specific examples of biotherapeutics include a monoclonal antibody, a bispecific antibody, an scFv, a Fab, a camelid, or a nanobody, e.g., adalimumab, infliximab, cetuximab, natalizumab, moxetumomab pasudotox, atezolizumab, nivolumab, abciximab, Brentuximab, Certolizumab pegol, elotuzumab, benralizumab, vedolizumab, galcanezumab, rituximab, alemtuzumab, dupilumab, golimumab, obinutuzumab, tildrakizumab, erenumab, mepolizumab, tamucirumab, ranibizumab, ustekinumab, reslizumab, ipilimumab, alirocumab, belimumab, panitumumab, avelumab, necitumumab, mogamulizumab, olaratumab, brodalumab, eculizumab, pertuzumab, pembrolizumab, or tocilizumab. In certain embodiments, the biotherapeutic is erythropoietin, thrombopoietin, human growth hormone, tissue factor, IFNβ-1b, IFNβ-1a, IL-2 or the IL-2 mimetic aldesleukin, exenatide, albiglutide, alefacept, palifermin, or belatacept.

In certain embodiments, the biotherapeutic is an enzyme, such as, asparaginase Erwinia chrysanthemi, phenylalanine ammonia-lyase, alpha-galactosidase A, acid α-glucosidase (GAA), glucocerebrosidase (GCase), aspartylglucosaminidase (AGA), alpha-L-iduronidase, iduronate sulfatase, sulfaminase, α-N-acetylglucosaminidase (NAGLU), heparin acetyle CoA: α-glucosaminide N-acetyltransferase (HGSNAT), N-acetylglucosamine 6-sulfatase (GNS), N-glucosamine 3-O-sulfatase (arylsulfatase G or ARSG), N-acetylgalactosamine 6-sulfatase, beta-galactosidase, N-acetylgalactosamine 4-sulfatase, beta-glucuronidase, Factor VIII, Factor IX, palmitoyl protein thioesterase (PPT1), Tripeptidyl peptidase (TPP1), Pseudomonas elastase (PaE), Pseudomonas alkaline protease (PaAP), or Streptococcal pyrogenic exotoxin B (SpeB). In certain embodiments, the biotherapeutic is not Factor VIII.

In some embodiments, an autoantigen, as provided herein, is a naturally occurring antigen in a healthy individual. Using PANTHER, (Protein ANalysis THrough Evolutionary Relationships), autoantigens can be classified into the following varieties based on function played by the antigen in-vivo that may have a role to play as, for example, an enzyme, intercellular adhesive protein, cell junction protein, cytoskeletal protein, extracellular matrix protein, cellular receptor, transcription or translational protein, gene editing protein, structural protein, or the like.

Autoantigens have been found to be associated with several autoimmune disorders. A comprehensive list of disorders and associated autoantigens can be found at the database AAgAtlas 1.0 database accessed by typing into a web browser http followed by: //biokb.ncpsb.org/followed by aagatlas/). Nonlimiting examples of autoantigens and their associated disorders include: p53-associated autoimmune diseases lupus and scleroderma; Desmoglein (Dsg) 1 and 3 autoantigens associated with Pemphigus vulgaris; autoantigens Gliadin and type 2 transglutaminase associated with Celiac disease; autoantigens PDC-E2 (Pyruvate dehydrogenase complex component E2) and BCOADC-E2 (Branched chain 2-oxo-acid dehydrogenase complex component E2) associated with Primary Biliary Cholangitis; autoantigens PLA2R (Phospholipase A2 Receptor) and THSD7A (thrombospondin type-1 domain containing protein 7A) associated with Membranous Nephropathy; autoantigen TSHR (Thyroid-Stimulating Hormone Receptor) associated with Graves' Disease; autoantigens AChR (Acetylcholine Receptor), MuSK (Muscle-Specific Kinase), and LRP4 (associated with Myasthenia Gravis; and autoantigens associated with rheumatoid arthritis (RA) (e.g., citrullinated peptides and proteins, carbamylated proteins, acetylated proteins) and systemic lupus erythematosus (SLE) (e.g., anti-nuclear antibodies, anti-dsDNA antibodies, anti-nucleosome antibodies, and others), and the like.

In certain aspects, an engineered autoantigen that suppresses an ongoing autoimmune response to the autoantigen in a subject includes a siglec ligand. The engineered autoantigen is configured to both bind to BCRs on a B cell that recognizes the autoantigen and to bind to a siglec present on the B cell. The autoantigen portion of the engineered autoantigen provides specificity for targeting only B cells that bind to the autoantigen while the siglec ligand, while not specific to a particular B cell clone, may bind to any B cell expressing the siglec, prevents activation of the B cell. In some instances, the siglec ligand provides a therapeutic benefit to the individual by suppressing the individual's immune response to the autoantigen, where the immune response is reduced by 50% or more when the engineered autoantigen is administered to an individual relative to when the individual is administered the non-engineered version of the autoantigen. Further, in some instances, the immune reaction is reduced by 60%, 70%, 80% or more, for example 85%, 90%, 95% or more, in certain cases 98%, 99%, or 100%, i.e., such that the immune response is undetectable, i.e., the autoantigen is rendered nonimmunogenic.

In some aspects of the disclosure, an engineered autoantigen is provided, wherein the engineered autoantigen (referred to interchangeably as the “clonally immunosuppressive autoantigen”, “hypoimmunogenic autoantigen”, “modified autoantigen” or simply “subject autoantigen”) is engineered to have an altered Sialic acid-binding immunoglobulin-type lectin (siglec) ligand (sigL) profile.

Thus, disclosed herein are engineered autoantigens which may retain the epitope(s) recognized by autoimmune antibodies, while comprising one or more modifications comprising addition of a sigL that render the autoantigen capable of suppressing an antigen-specific immune response in an individual to which it has been administered as compared to the unmodified autoantigen. In some embodiments, the immune response is a humoral immune response i.e., a B cell-driven response, e.g., an IgG response.

### Engineered and Hypoimmunogenic

The term “engineered” refers to a biotherapeutic that has been designed and built to comprise one or more modifications relative to biotherapeutic that has not been so engineered, i.e. a parental biotherapeutic, which is also referred to herein as an unengineered biotherapeutic.

By a “hypoimmunogenic” composition, it is meant a composition that suppresses an unwanted, drug-specific immune response in an individual relative to a reference composition, e.g. a corresponding nonengineered composition, when administered to the individual; for example, reducing an immune response by 50% or more relative to a reference, e.g. a nonengineered biotherapeutic, in some instances 60%, 70%, 80% or more, for example 85%, 90%, 95% or more, in certain cases 98%, 99%, or 100%, i.e. such that the immune response is undetectable, i.e. the biotherapeutic is nonimmunogenic. Thus, disclosed herein are engineered biotherapeutics which retain pharmacologic activity while comprising one or more modifications that render the biotherapeutic capable of suppressing a drug-specific immune response in an individual to which it has been administered as compared to the unmodified biotherapeutic. In some embodiments, the immune response is a humoral immune response i.e., a B cell-driven response, e.g. an IgG response.

### Pharmaceutical Compositions

Provided are pharmaceutical compositions comprising:
a conjugate as described herein; and
a pharmaceutical acceptable excipient.

In some cases, the composition includes a racemic mixture of stereoisomers. In some embodiments, the composition is enriched in a particular stereoisomer, e.g. the composition is enriched in a first enantiomer relative to a second enantiomer. The term “enantiomeric excess” is used herein to quantify the relative amount of the first enantiomer compared to the second enantiomer, wherein enantiomeric excess is the absolute difference between the mole fraction of each enantiomer. For instance, if 70% of a compound is a first enantiomer and 30% of the compound is the second enantiomer, then the enantiomeric excess is 40% (i.e. 70% minus 40%). In some cases, the composition has an enantiomeric excess of the first enantiomer of 1% or more, such as 10% or more, 20% or more, 30% or more, or 40% or more. In some embodiments, the composition is an aqueous solution of the compound.

In certain embodiments, the disclosed conjugates are useful for the treatment of a disease or disorder. Accordingly, pharmaceutical compositions comprising at least one disclosed conjugate are also described herein. For example, the present disclosure provides pharmaceutical compositions that include a therapeutically effective amount of a conjugate of the present disclosure (or a pharmaceutically acceptable salt or solvate or hydrate or stereoisomer thereof) and a pharmaceutically acceptable excipient.

A pharmaceutical composition that includes a subject conjugate may be administered to a patient alone, or in combination with other supplementary active agents. For example, one or more conjugates according to the present disclosure can be administered to a patient with or without supplementary active agents. The pharmaceutical compositions may be manufactured using any of a variety of processes, including, but not limited to, conventional mixing, dissolving, granulating, dragee-making, levigating, emulsifying, encapsulating, entrapping, lyophilizing, and the like. The pharmaceutical composition can take any of a variety of forms including, but not limited to, a sterile solution, suspension, emulsion, spray dried dispersion, lyophilisate, tablet, microtablets, pill, pellet, capsule, powder, syrup, elixir or any other dosage form suitable for administration.

In methods of treating an individual with the subject hypoimmunogenic biotherapeutic, the patient will typically be administered a pharmaceutical composition comprising the subject hypoimmunogenic biotherapeutic. By a pharmaceutical composition, it is meant an engineered hypoimmunogenic biotherapeutic of the present disclosure that has been formulated in a pharmaceutically acceptable carrier. As used herein, a “pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

The pharmaceutical compositions of the disclosure are administered at a therapeutically effective dosage, e.g., a dosage sufficient to provide treatment for the disease states previously described. Administration of the compounds of the disclosure or the pharmaceutically acceptable salts thereof can be via any of the accepted modes of administration for agents that serve similar utilities. While human dosage levels have yet to be optimized for the compounds of the disclosure, these can be readily extrapolated from doses administered to a relevant animal model, e.g. mice that results in treatment of the disease or disorder in that animal model. Generally, an individual human dose is from about 0.01 to 2.0 mg/kg of body weight, preferably about 0.1 to 1.5 mg/kg of body weight, and most preferably about 0.3 to 1.0 mg/kg of body weight. Treatment can be administered for a single day or a period of days, and can be repeated at intervals of several days, one or several weeks, or one or several months. Administration can be as a single dose (e.g., as a bolus) or as an initial bolus followed by continuous infusion of the remaining portion of a complete dose over time, e.g., 1 to 7 days. The amount of active compound administered will, of course, be dependent on any or all of the following: the subject and disease state being treated, the severity of the affliction, the manner and schedule of administration and the judgment of the prescribing physician. It will also be appreciated that amounts administered will depend upon the molecular weight of the biotherapeutic, the amount of siglec ligand covalently bound, and the size of the linker.

While all typical routes of administration are contemplated (e.g. oral, topical, transdermal, injection (intramuscular, intravenous, or intra-arterial)), it is presently preferred to provide liquid dosage forms suitable for injection. Generally, depending on the intended mode of administration, the pharmaceutically acceptable composition will contain about 0.1% to 95%, preferably about 0.5% to 50%, by weight of the subject hypoimmunogenic biotherapeutic of the disclosure, the remainder being suitable pharmaceutical excipients, carriers, etc. Dosage forms or compositions containing active ingredient in the range of 0.005% to 95% with the balance made up from non-toxic carrier can be prepared.

The subject pharmaceutical compositions can be administered either alone or in combination with other pharmaceutical agents. These compositions can include other medicinal agents, pharmaceutical agents, carriers, and the like, including, but not limited to other active agents that can act as immune-modulating agents and more specifically can have inhibitory effects on B-cells, including anti-folates, immune suppressants, cyostatics, mitotic inhibitors, and anti-metabolites, or combinations thereof.

Liquid pharmaceutically administrable compositions can, for example, be prepared by dissolving, dispersing, etc. an active composition of the disclosure (e.g., a lyophilized powder) and optional pharmaceutical adjuvants in a carrier, such as, for example, water (water for injection), saline, aqueous dextrose, glycerol, glycols, ethanol or the like (excluding galactoses), to thereby form a solution or suspension. If desired, the pharmaceutical composition to be administered can also contain minor amounts of nontoxic auxiliary substances such as wetting agents, emulsifying agents, stabilizing agents, solubilizing agents, pH buffering agents and the like, for example, sodium acetate, sodium citrate, cyclodextrine derivatives, sorbitan monolaurate, triethanolamine acetate and triethanolamine oleate, etc., osmolytes, amino acids, sugars and carbohydrates, proteins and polymers, salts, surfactants, chelators and antioxidants, preservatives, and specific ligands. Actual methods of preparing such dosage forms are known, or will be apparent, to those skilled in this art; for example, see Remington: The Science and Practice of Pharmacy, Pharmaceutical Press, 22nd Edition, 2012. The composition or formulation to be administered will, in any event, contain a quantity of the active compound in an amount effective to treat the symptoms of the subject being treated.

### Methods of Treatment

Provided are methods of treating a subject for a condition by administering a conjugate as described herein. In some cases, the subject has been diagnosed with a condition.

The subject compounds or prodrugs find use for treating a disease or disorder in a subject. The route of administration may be selected according to a variety of factors including, but not limited to, the condition to be treated, the formulation and/or device used, the subject to be treated, and the like. Routes of administration useful in the disclosed methods include, but are not limited to, oral and parenteral routes, such as intravenous (iv), intraperitoneal (ip), rectal, topical, ophthalmic, nasal, otic, intrathecal, and transdermal. Formulations for these dosage forms are described herein.

An effective amount of a subject compound or prodrug may depend, at least, on the particular method of use, the subject being treated, the severity of the affliction, and the manner of administration of the therapeutic composition. A “therapeutically effective amount” of a composition is a quantity of a specified compound or prodrug sufficient to achieve a desired effect in a subject (e.g., patient) being treated. For example, this may be the amount of a subject compound necessary to prevent, inhibit, reduce or relieve a disease or disorder in a subject. Ideally, a therapeutically effective amount of a compound or prodrug is an amount sufficient to prevent, inhibit, reduce or relieve a disease or disorder in a subject without causing a substantial cytotoxic effect on host cells in the subject.

Therapeutically effective doses of a subject compound or prodrug or pharmaceutical composition can be determined by one of skill in the art. For example, in some instances, a therapeutically effective dose of a compound or prodrug or pharmaceutical composition is administered with a goal of achieving local (e.g., tissue) concentrations that are at least as high as the IC₅₀ of an applicable compound disclosed herein.

The specific dose level and frequency of dosage for any particular subject may be varied and may depend upon a variety of factors, including the activity of the subject compound or prodrug, the metabolic stability and length of action of that compound or prodrug, the age, body weight, general health, sex and diet of the subject, mode and time of administration, rate of excretion, drug combination, and severity of the condition of the host undergoing therapy.

In some embodiments, multiple doses of a compound or prodrug are administered. The frequency of administration of a compound can vary depending on any of a variety of factors, e.g., severity of the symptoms, condition of the subject, etc. For example, in some embodiments, a compound is administered once per month, twice per month, three times per month, every other week, once per week (qwk), twice per week, three times per week, four times per week, five times per week, six times per week, every other day, daily (qd/od), twice a day (bds/bid), or three times a day (tds/tid), etc.

### Methods of Manufacture

Also provided are methods of making the conjugates disclosed herein. Provided is a method of making a conjugate, comprising:
covalently attaching a siglec ligand as described herein to a biologically active substance, thereby making the conjugate.

As discussed above, in some cases the conjugate includes two or more siglec ligands. In some cases, the conjugate includes two or more siglec ligands that are each covalently bonded to a connector (C), and the connector is covalently bonded to the biologically active substance. In such cases, the method includes covalently bonding each of the two or more siglec ligands to the connector and covalently bonding the connector to the biologically active substance. The covalently bonding of the siglec ligands can happen first, or the covalently bonding to the biologically active substance can happen first.

Methods of covalently binding siglec ligands to biologically active substances (e.g. biotherapeutics) or connectors are well appreciated in the art, any of which may be deployed to modify a biotherapeutic of choice to become an engineered hypoimmunogenic biotherapeutic of the present disclosure. For example, the modification may be performed by engineered biosynthesis. By “biosynthesis”, it is meant a synthesis process that is mediated by cells. For example, in the Golgi apparatus, a subset of the 20 known sialyltransferases attach sialic acids to underlying monosaccharides such as galactose via three different types of linkage (α2,3, α2,6, and α2,8). By engineered biosynthesis, it is meant a synthesis process that is mediated by cells that have been engineered to perform the process, in some instances de novo, in other instances, in a modified way. Thus, for example, a producer cell line may be genetically engineering to express one or more sialyl transferases, e.g. sialyltransferase (EC 2.4.99), beta-galactosamide alpha-2,6-sialyltransferase (EC 2.4.99.1), alpha-N-acetylgalactosaminide alpha-2,6-sialyltransferase (EC 2.4.99.3), beta-galactoside alpha-2,3-sialyltransferase (EC 2.4.99.4), N-acetyllactosaminide alpha-2,3-sialyltransferase (EC 2.4.99.6), alpha-N-acetyl-neuraminide alpha-2,8-sialyltransferase (EC 2.4.99.8); lactosylceramide alpha-2,3-sialyltransferase (EC 2.4.99.9), or other enzymes in an enzymatic pathway, e.g. CMP-Neu5Ac hydroxylase, sialate-4-O-acetyl transferase, sialate-4-O-acetylesterase, sialate-7(9)-O-acetyltransferase, sialate-8-O-methyl transferase, sialate-9-)-acetyltransferase, etc. that drives the covalent binding of a specific sialic acid to the biotherapeutic or that targets specific novel amino acid residues for covalent modification with sialic acid. As another example, a producer cell line could be fed a precursor substrate that will be incorporated by the producer line into the manufactured biotherapeutic as a specific siglec ligand. Any producer cell that finds use in the expression of proteins for use as therapeutic biotherapeutics may be used in this process, for example a mammalian cell (CHO, HEK, etc.), an insect cell (SF9, etc.), a bacterium, a protozoan (Leishmania, etc.). as disclosed in, e.g. WO2017093291, WO2019002512, WO2019234021, the full disclosures of which are incorporated herein in their entirety by reference.

As another example, the modification may be performed by chemical conjugation. By “chemical conjugation”, it is meant a process that occurs exogenous to a cell. Thus, for example, the siglec ligand might be enzymatically or chemically linked to the biotherapeutic after biosynthesis from producer cell line. Nonlimiting examples of such in vitro processes are disclosed in U.S. Pat. Nos. 7,220,555, 6,376,475B, and 5,409,817, the full disclosures of which are incorporated herein by reference. In some such embodiments, a connector may be deployed to covalently link the sialic acid to the biotherapeutic. Many examples of connector exist in the art, any of which may be used to chemically conjugate sialic acid(s) to the biotherapeutic to arrive at hypoimmunogenic biotherapeutics of the present disclosure.

As a third example, specifically directed to embodiments in which the siglec ligand is a peptide or polypeptide sequence, e.g. an scFv or peptide derived from epratuzumab, e.g. PV1, PV2 or PV3, the modification may be performed by genetic engineering of the biotherapeutic to comprise the peptide/polypeptide sequence within the biotherapeutic. For example, the polynucleotide used to produce the biotherapeutic may be modified by standard molecular biology cloning techniques to include a polynucleotide sequence encoding the peptide/polypeptide in the same translational reading frame (“In frame”), such that upon transcription and translation of the biotherapeutic in a producing cell, the biotherapeutic will comprise the peptide/polypeptide sequence covalently associated with amino acids that make up the biotherapeutic, resulting in a biotherapeutic that is hypoimmunogenic. Preferably, the peptide/polypeptide sequence will be genetically engineered into a domain of the biotherapeutic that is not responsible for the therapeutic effect of the biotherapeutic, e.g. the enzymatic domain of an enzyme, the Fab or more specifically CDR domains of an antibody, etc. In the instance of modifying a viral particle, the peptide/polypeptide sequence will preferably be genetically engineered into a capsid or envelop protein so as to be exposed to the exterior of the viral particle, e.g. into an exposed loop of a viral capsid protein, a surface-exposed tegument protein, etc. Such structural features are well understood by one of ordinary skill in the art of viral therapies.

### Methods of Use

Provided are methods of using the conjugates. Provided is a method of treating a patient for a condition, the method comprising:
administering to the patient a conjugate as described herein.

The hypoimmunogenic compositions of the present disclosure find particular use in the treatment of diseases that require repeat or chronic administration of the therapeutic to be effective. There are many instances of such conditions, of which a few nonlimiting examples are provided below and elsewhere. It is expected that the ordinarily skilled artisan will be able to extrapolate from these examples to other indications and biotherapeutics as known in the art.

For example, the individual may be suffering from a chronic autoimmune or inflammatory disease, e.g. rheumatoid arthritis, psoriatic arthritis, ankylosing spondylitis, Crohn's disease, ulcerative colitis, psoriasis, hidradenitis suppurativa, uveitis, and juvenile idiopathic arthritis. In such instances, the method may comprise administering to the individual a hypoimmunogenic TNFα-specific antibody, e.g. a hypoimmunogenic adalimumab engineered from adalimumab, or a hypoimmunogenic infliximab engineered from infliximab, in an amount effective to treat the chronic immune disease.

As another example, the individual may be suffering from a leukemia, e.g. ALL. In such instances, the method may comprise administering to the individual an engineered hypoimmunogenic asparaginase from Erwinia chrysanthemi in an amount effective to treat the leukemia.

As another example, the individual may be suffering from a colorectal cancer, a non-small cell lung cancer, or a head and neck cancer. In such instances, the method may comprise administering to the individual an engineered hypoimmunogenic cetuximab in an amount effective to treat the colorectal cancer, non-small cell lung cancer, or head and neck cancer.

As another example, the individual may be suffering from multiple sclerosis. In such instances, the method may comprise administering to the individual an engineered hypoimmunogenic natalizumab, an engineered hypoimmunogenic IFNβ-1b, or an engineered hypoimmunogenic IFNβ-1a in an amount effective to treat the multiple sclerosis.

As another example, the individual may be the recipient of an organ transplant and in need of an immunosuppressive agent that protects the transplanted tissue from rejection by the individual's immune system. In such instances, the method may comprise administering to the individual an engineered hypoimmunogenic IdeS in an amount effective to prevent an antibody response to the transplanted tissue. In some embodiments, the transplanted organ is an allogeneic graft. In some embodiments, the transplanted organ is a xenogeneic graft. In some embodiments, the organ is selected from kidney, heart, lung, liver, pancreas, trachea, vascular tissue, skin, bone, cartilage, adrenal tissue, fetal thymus, and cornea.

As another example, the individual may be suffering from Type 2 Diabetes. In such instances, the method would comprise administering to the individual an engineered hypoimmunogenic exenatide or engineered hypoimmunogenic albiglutide in an amount effective to treat the diabetes.

As another example, the individual may be suffering from a complement-mediated disease. In such instances, the method would comprise administering to the individual an engineered hypoimmunogenic complement degrading protease, e.g. from a pathogen such as a bacterial pathogen or fungal pathogen (e.g. Pseudomonas Elastase (PaE), Pseudomonas Alkaline protease (PaAP), Streptococcal pyrogenic Exotoxin B (SpeB), a gingipain from Porphyromonas gingivalis, Aspergillus Alkaline protease 1 (Alp1), C. albicans Secreted aspartyl proteinases 1 (Sap1), 2 (Sap2), and 3 (Sap3), in an amount effective to degrade complement and treat the disease.

As another example, the individual may be suffering from an enzyme deficiency. In such instances, the method would comprise administering to the individual an engineered hypoimmunogenic enzyme in an amount effective to treat the deficiency. Nonlimiting examples of such enzyme deficiencies would include PKU, wherein a hypoimmunogenic phenylalanine ammonia-lyase would be administered; Fabry disease, wherein a hypoimmunogenic alpha-galactosidase A would be administered; Pompe disease, wherein a hypoimmunogenic acid α-glucosidase (GAA) would be administered; Gaucher disease, wherein a hypoimmunogenic glucocerebrosidase (GCase) would be administered; Aspartylglucosaminuria, wherein a hypoimmunogenic aspartylglucosaminidase (AGA) would be administered; Hypophosphatasia (HPP), wherein a hypoimmunogenic asfotase would be administered; MPS I, wherein a hypoimmunogenic alpha-L-iduronidase would be administered; MPS II, wherein a hypoimmunogenic iduronate sulfatase would be administered; MPS IIIa, wherein a hypoimmunogenic sulfaminase would be administered; MPS IIIB, wherein a hypoimmunogenic α-N-acetylglucosaminidase (NAGLU) would be administered; MPS IIIC, wherein a hypoimmunogenic heparin acetyle CoA: α-glucosaminide N-acetyltransferase (HGSNAT) would be administered; MPS IIID, wherein a hypoimmunogenic N-acetylglucosamine 6-sulfatase (GNS) would be administered; MPS IIIE, wherein a hypoimmunogenic N-glucosamine 3-O-sulfatase (arylsulfatase G or ARSG) would be administered; MPS IVA, wherein a hypoimmunogenic N-acetylgalactosamine 6-sulfatase would be administered; MPS IVB, wherein a hypoimmunogenic beta-galactosidase would be administered; MPS VI, wherein a hypoimmunogenic N-acetylgalactosamine 4-sulfatase would be administered; MPS VI, wherein a hypoimmunogenic beta-glucuronidase would be administered; Hemophilia A, wherein a hypoimmunogenic Factor VIII would be administered; Hemophilia B, wherein a hypoimmunogenic Factor IX would be administered; the CLN1 form of Batten Disease, wherein a hypoimmunogenic palmitoyl protein thioesterase (PPT1) would be administered; the CLN2 form of Batten Disease, wherein a hypoimmunogenic Tripeptidyl peptidase (TPP1) would be administered; arginase-1 deficiency, wherein a hypoimmunogenic arginase-1 or pegzilarginase would be administered; and cystathionine beta synthase (CBS) deficiency, also known as Classical Homocystinuria, wherein a hypoimmunogenic cystathionine beta synthase or Aeglea product AGLE-177 is administered.

As another example, the individual may be suffering from disease that would benefit from a gene therapy, e.g. a genetic disease, or a complex disease (i.e. not restricted to being associated with a specific genetic etiology) in which chronic expression of a therapeutic RNA or protein would treat the condition. In such instances, the method would comprise administering to the individual an engineered hypoimmunogenic viral particle comprising a polynucleotide sequence (a “transgene”) encoding the therapeutic gene product of interest, in an amount effective to treat the disease. Nonlimiting examples of suitable transgenes/gene products that one might deliver via the subject hypoimmunogenic viral particle include those associated with muscular dystrophy, cystic fibrosis, familial hypercholesterolemia, and rare or orphan diseases. Examples of such rare disease may include spinal muscular atrophy (SMA), Huntingdon's Disease, Rett Syndrome (e.g., methyl-CpG-binding protein 2 (MeCP2); UniProtKB-P51608), Amyotrophic Lateral Sclerosis (ALS), Duchenne Type Muscular dystrophy, Friedrichs Ataxia (e.g., frataxin), ATXN2 associated with spinocerebellar ataxia type 2 (SCA2)/ALS; TDP-43 associated with ALS, progranulin (PRGN) (associated with non-Alzheimer's cerebral degenerations, including, frontotemporal dementia (FTD), progressive non-fluent aphasia (PNFA) and semantic dementia), among others. See, e.g., www.orpha.net/consor/cgi-bin/Disease_Search_List.php; rarediseases.info.nih.gov/diseases.

Other useful therapeutic gene products that could be encoded by the transgene also include hormones and growth and differentiation factors including, without limitation, insulin, glucagon, glucagon-like peptide 1 (GLP-1), growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO), connective tissue growth factor (CTGF), basic fibroblast growth factor (bFGF), acidic fibroblast growth factor (aFGF), epidermal growth factor (EGF), transforming growth factor a (TGFa), platelet-derived growth factor (PDGF), insulin growth factors I and II (IGF-I and IGF-II), any one of the transforming growth factor b superfamily, including TGF b, activins, inhibins, or any of the bone morphogenic proteins (BMP) BMPs 1-15, any one of the heregluin/neuregulin/ARIA/neu differentiation factor (NDF) family of growth factors, nerve growth factor (NGF), brain-derived neurotrophic factor (BDNF), neurotrophins NT-3 and NT-4/5, ciliary neurotrophic factor (CNTF), glial cell line derived neurotrophic factor (GDNF), neurturin, agrin, any one of the family of semaphorins/collapsins, netrin-1 and netrin-2, hepatocyte growth factor (HGF), ephrins, noggin, sonic hedgehog and tyrosine hydroxylase.

Other useful transgenes include those that encode proteins that regulate the immune system including, without limitation, cytokines and lymphokines such as thrombopoietin (TPO), interleukins (IL) IL-1 through IL-25 (including, IL-2, IL-4, IL-12, and IL-18), monocyte chemoattractant protein, leukemia inhibitory factor, granulocyte-macrophage colony stimulating factor, Fas ligand, tumor necrosis factors a and b, interferons a, b, and g, stem cell factor, Hk-2/flt3 ligand. Gene products produced by the immune system are also useful in the invention. These include, without limitations, immunoglobulins IgG, IgM, IgA, IgD and IgE, chimeric immunoglobulins, humanized antibodies, single chain antibodies, T cell receptors, chimeric T cell receptors, single chain T cell receptors, class I and class II MHC molecules, as well as engineered immunoglobulins and MHC molecules. Useful gene products also include complement regulatory proteins such as complement regulatory proteins, membrane cofactor protein (MCP), decay accelerating factor (DAF), CR1, CF2 and CD59.

Still other useful transgenes include those that encode gene products for any one of the receptors for the hormones, growth factors, cytokines, lymphokines, regulatory proteins and immune system proteins.

Still other useful transgenes include those encoding receptors for cholesterol regulation, including the low density lipoprotein (LDL) receptor, high density lipoprotein (HDL) receptor, the very low density lipoprotein (VLDL) receptor, and the scavenger receptor. The invention also encompasses gene products such as members of the steroid hormone receptor superfamily including glucocorticoid receptors and estrogen receptors, Vitamin D receptors and other nuclear receptors. In addition, useful gene products include transcription factors such as jun,fos, max, mad, serum response factor (SRF), AP-1, AP2, myb, MyoD and myogenin, ETS-box containing proteins, TFE3, E2F, ATF1, ATF2, ATF3, ATF4, ZF5, NFAT, CREB, HNF-4, C/EBP, SP1, CCAAT-box binding proteins, interferon regulation factor (IRF-1), Wilms tumor protein, ETS-binding protein, STAT, GATA-box binding proteins, e.g., GATA-3, and the forkhead family of winged helix proteins.

Other useful gene products include, carbamoyl synthetase I, ornithine transcarbamylase, arginosuccinate synthetase, arginosuccinate lyase, arginase, fumarylacetacetate hydrolase, phenylalanine hydroxylase, alpha-1 antitrypsin, glucose-e-phosphatase, porphobilinogen deaminase, Factor VIII, Factor IX, cystathione beta-synthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylate, hepatic phosphorylase, phosphorylase kinase, glycine decarboxylase, H-protein, T-protein, a cystic fibrosis transmembrane regulator (CFTR) sequence, and a dystrophin sequence or functional fragment thereof. Still other useful gene products include enzymes such as may be useful in enzyme replacement therapy, which is useful in a variety of conditions resulting from deficient activity of enzyme. For example, enzymes that contain mannose-6-phosphate may be utilized in therapies for lysosomal storage diseases (e.g., a suitable gene includes that encodes b-glucuronidase (GUSB)). In another example, the gene product is ubiquitin protein ligase E3A (UBE3A). Still useful gene products include UDP Glucuronosyltransferase Family 1 Member A1 (UGT1A1).

In some embodiment, the gene product is not Factor VIII.

Other useful gene products include non-naturally occurring polypeptides, such as chimeric or hybrid polypeptides having a non-naturally occurring amino acid sequence containing insertions, deletions or amino acid substitutions. For example, single-chain engineered immunoglobulins could be useful in certain immunocompromised patients. Other types of non-naturally occurring gene sequences include antisense molecules and catalytic nucleic acids, such as ribozymes, which could be used to reduce overexpression of a target.

Reduction and/or modulation of expression of a gene is particularly desirable for treatment of hyperproliferative conditions characterized by hyperproliferating cells, as are cancers and psoriasis. Target polypeptides include those polypeptides which are produced exclusively or at higher levels in hyperproliferative cells as compared to normal cells. Target antigens include polypeptides encoded by oncogenes such as myb, myc, fyn, and the translocation gene bcr/abl, ras, src, P53, neu, trk and EGRF. In addition to oncogene products as target antigens, target polypeptides for anti-cancer treatments and protective regimens include variable regions of antibodies made by B cell lymphomas and variable regions of T cell receptors of T cell lymphomas which, in some embodiments, are also used as target antigens for autoimmune disease. Other tumor-associated polypeptides can be used as target polypeptides such as polypeptides which are found at higher levels in tumor cells including the polypeptide recognized by monoclonal antibody 17-1A and folate binding polypeptides.

Other suitable transgenes include those which encode therapeutics that may be useful for treating individuals suffering from autoimmune diseases and disorders by conferring a broad based protective immune response against targets that are associated with autoimmunity including cell receptors and cells which produce self-directed antibodies. T cell mediated autoimmune diseases include Rheumatoid arthritis (RA), multiple sclerosis (MS), Sjogren's syndrome, sarcoidosis, insulin dependent diabetes mellitus (IDDM), autoimmune thyroiditis, reactive arthritis, ankylosing spondylitis, scleroderma, polymyositis, dermatomyositis, psoriasis, vasculitis, Wegener's granulomatosis, Crohn's disease and ulcerative colitis. Each of these diseases is characterized by T cell receptors (TCRs) that bind to endogenous antigens and initiate the inflammatory cascade associated with autoimmune diseases.

Still other useful gene products include those used for treatment of hemophilia, including hemophilia B (including Factor IX) and hemophilia A (including Factor VIII and its variants, such as the light chain and heavy chain of the heterodimer and the B-deleted domain; U.S. Pat. Nos. 6,200,560 and 6,221,349). In some embodiments, the minigene comprises first 57 base pairs of the Factor VIII heavy chain which encodes the 10 amino acid signal sequence, as well as the human growth hormone (hGH) polyadenylation sequence. In alternative embodiments, the minigene further comprises the A1 and A2 domains, as well as 5 amino acids from the N-terminus of the B domain, and/or 85 amino acids of the C-terminus of the B domain, as well as the A3, C1 and C2 domains. In yet other embodiments, the nucleic acids encoding Factor VIII heavy chain and light chain are provided in a single mini gene separated by 42 nucleic acids coding for 14 amino acids of the B domain [U.S. Pat. No. 6,200,560]

Further illustrative genes which may be delivered via the hypoimmunogenic viral particle include, without limitation, glucose-6-phosphatase, associated with glycogen storage disease or deficiency type 1A (GSD1), phosphoenolpyruvate-carboxy kinase (PEPCK), associated with PEPCK deficiency; cyclin-dependent kinase-like 5 (CDKL5), also known as serine/threonine kinase 9 (STK9) associated with seizures and severe neurodevelopmental impairment; galactose-1 phosphate uridyl transferase, associated with galactosemia; phenylalanine hydroxylase (PAH), associated with phenylketonuria (PKU); gene products associated with Primary Hyperoxaluria Type 1 including Hydroxy acid Oxidase 1 (GO/HAO1) and AGXT, branched chain alpha-ketoacid dehydrogenase, including BCKDH, BCKDH-E2, BAKDH-E1a, and BAKDH-E1b, associated with Maple syrup urine disease; fumarylacetoacetate hydrolase, associated with tyrosinemia type 1; methylmalonyl-CoA mutase, associated with methylmalonic acidemia; medium chain acyl CoA dehydrogenase, associated with medium chain acetyl CoA deficiency; ornithine transcarbamylase (OTC), associated with ornithine transcarbamylase deficiency; argininosuccinic acid synthetase (ASS1), associated with citrullinemia; lecithin-cholesterol acyltransferase (LCAT) deficiency; amethylmalonic acidemia (MMA); NPC1 associated with Niemann-Pick disease, type Cl); propionic academia (PA); TTR associated with Transthyretin (TTR)-related Hereditary Amyloidosis; low density lipoprotein receptor (LDLR) protein, associated with familial hypercholesterolemia (FH), LDLR variant, such as those described in WO 2015/164778; PCSK9; ApoE and ApoC proteins, associated with dementia; UDP-glucouronosyltransferase, associated with Crigler-Najjar disease; adenosine deaminase, associated with severe combined immunodeficiency disease; hypoxanthine guanine phosphoribosyl transferase, associated with Gout and Lesch-Nyan syndrome; biotimidase, associated with biotimidase deficiency; alpha-galactosidase A (a-Gal A) associated with Fabry disease); beta-galactosidase (GLB1) associated with GM1 gangliosidosis; ATP7B associated with Wilson's Disease; beta-glucocerebrosidase, associated with Gaucher disease type 2 and 3; peroxisome membrane protein 70 kDa, associated with Zellweger syndrome; arylsulfatase A (ARSA) associated with metachromatic leukodystrophy, galactocerebrosidase (GALC) enzyme associated with Krabbe disease, alpha-glucosidase (GAA) associated with Pompe disease; sphingomyelinase (SMPD1) gene associated with Nieman Pick disease type A; argininosuccsinate synthase associated with adult onset type II citrullinemia (CTLN2); carbamoyl-phosphate synthase 1 (CPS1) associated with urea cycle disorders; survival motor neuron (SMN) protein, associated with spinal muscular atrophy; ceramidase associated with Farber lipogranulomatosis; b-hexosaminidase associated with GM2 gangliosidosis and Tay-Sachs and Sandhoff diseases; aspartylglucosaminidase associated with aspartyl-glucosaminuria; a-fucosidase associated with fucosidosis; a-mannosidase associated with alpha-mannosidosis; porphobilinogen deaminase, associated with acute intermittent porphyria (AIP); alpha-1 antitrypsin for treatment of alpha-1 antitrypsin deficiency (emphysema); erythropoietin for treatment of anemia due to thalassemia or to renal failure; vascular endothelial growth factor, angiopoietin-1, and fibroblast growth factor for the treatment of ischemic diseases; thrombomodulin and tissue factor pathway inhibitor for the treatment of occluded blood vessels as seen in, for example, atherosclerosis, thrombosis, or embolisms; aromatic amino acid decarboxylase (AADC), and tyrosine hydroxylase (TH) for the treatment of Parkinson's disease; the beta adrenergic receptor, anti-sense to, or a mutant form of, phospholamban, the sarco(endo)plasmic reticulum adenosine triphosphatase-2 (SERCA2), and the cardiac adenylyl cyclase for the treatment of congestive heart failure; a tumor suppressor gene such as p53 for the treatment of various cancers; a cytokine such as one of the various interleukins for the treatment of inflammatory and immune disorders and cancers; dystrophin or minidystrophin and utrophin or miniutrophin for the treatment of muscular dystrophies; and, insulin or GLP-1 for the treatment of diabetes.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for.

### General Synthetic Procedures

Many general references providing commonly known chemical synthetic schemes and conditions useful for synthesizing the disclosed compounds are available (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978).

During any of the processes for preparation of the subject compounds, it may be necessary and/or desirable to protect sensitive or reactive groups on any of the molecules concerned. This may be achieved by means of conventional protecting groups as described in standard works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie”, Houben-Weyl, 4th edition, Vol. 15/1, Georg Thieme Verlag, Stuttgart 1974, in H.-D. Jakubke and H. Jescheit, “Aminosauren, Peptide, Proteine”, Verlag Chemie, Weinheim, Deerfield Beach, and Basel 1982, and/or in Jochen Lehmann, “Chemie der Kohlenhydrate: Monosaccharide and Derivate”, Georg Thieme Verlag, Stuttgart 1974. The protecting groups may be removed at a convenient subsequent stage using methods known from the art.

The subject compounds can be synthesized via a variety of different synthetic routes using commercially available starting materials and/or starting materials prepared by conventional synthetic methods. A variety of examples of synthetic routes that can be used to synthesize the compounds disclosed herein are described in the schemes below.

All synthetic chemistry was performed in standard laboratory glassware, under inert atmosphere and anhydrous conditions unless indicated otherwise in the examples. Commercial reagents were used as received.

Analytical LC/MS was performed either on a Shimadzu LCMS-2020 equipped with a Phenomenex Jupiter@3 μm C18 300 Å, LC Column 50×2 mm, using LabSolutions software. Retention times were determined from the extracted 214 and/or 254 nm UV chromatogram or an Agilent 1290 Infinity RRLC attached with Agilent 6120 Mass detector and Diode array Detector and equipped with a YMC Triart C18 (150×4.6 mm), 5 μm column. Unless specified, the LC/MS analytical method on the Shimadzu used a gradient 5-100% acetonitrile in water (+0.1% TFA) over 4.25 mins. LC/MS analytical method on the Agilent 6120 used a gradient 10-100% acetonitrile in water (+0.05% TFA) over 11 mins. HPLC was performed on an Agilent 1260 infinity series HPLC with PDA detector equipped with a YMC Triart C18 (150×4.6 mm), 5 μm column. Analytical method on the Agilent 1260 used a gradient 10-95% acetonitrile in water (+0.1% TFA) over 10 mins. Prep HPLC was performed either on a Teledyne ISCO ACCQPrep™ HP150 system (with 200-400 nm—UV PDA and ELSD detector) equipped with a Phenomenex Gemini@10 μm NX—C18 110 Å, LC column 250×50 mm, AXIA, a Gilson system using a 215 liquid handler, 333 and 334 pumps, UV/VIS-155 detector equipped with the same type of column and Trilution Ic software, or a Shimadzu LC 20 AP with SPD 20 A UV Detector equipped with a Waters™ Sunfire C18 (250×30 mm), 5 μm column. Unless specified, prep HPLC purification used a gradient acetonitrile in water (+0.1% TFA). ¹H NMR was performed either on a Bruker 400 MHz Advance, a Bruker 500 MHz or a Bruker Fourier 300 MHz using Topspin software. Analytical thin layer chromatography was performed on silica (Sigma Aldrich TLC Silica gel 60 F254 aluminum or glass TLC plate, silica gel coated with fluorescent indicator F254) and was visualized under UV light then stained with either potassium permanganate or phosphomolybdic acid stain solutions. Silica gel chromatography was performed with Teledyne ISCO CombiFlash Rf+ automated chromatography for gradient elution. Removal of aqueous and high boiling point solvents was usually performed by lyophilization using a Labconco Freezone® 4.5 Plus −84° C. connected to a Labconco Rotary Vane Vacuum Pump, 195 LPM.

### Example 1: Synthesis of Compounds

### Intermediate A

### Methyl (4S,5R,6R)-5-(2-acetoxyacetamido)-6-((1R,2R)-3-amino-1,2-dihydroxypropyl)-4-hydroxy-2-(p-tolylthio)tetrahydro-2H-pyran-2-carboxylate (Int.A)

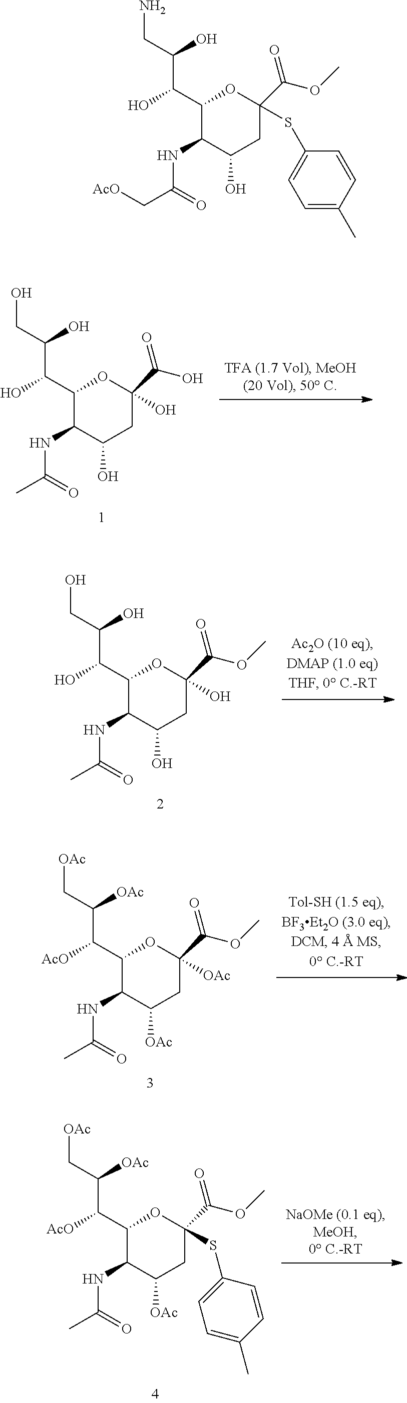

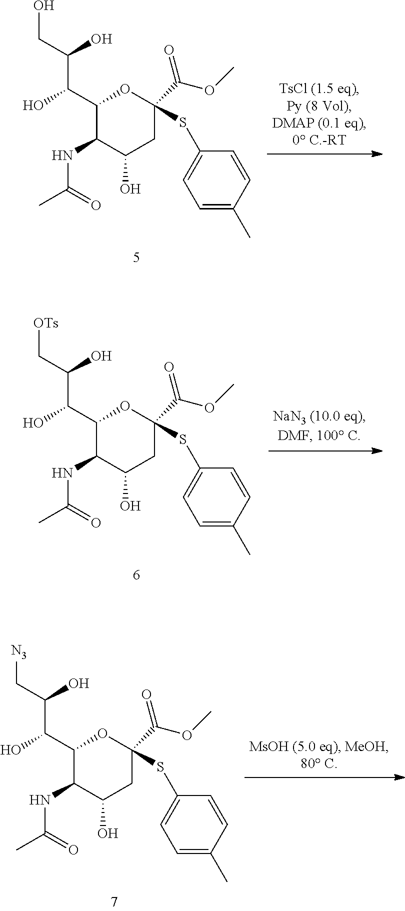 [SMILES: COC(=O)[C@]1(Sc2ccc(C)cc2)C[C@H](O)[C@@H](NC(C)=O)[C@H]([C@H](O)[C@H](O)CO)O1 ; COC(=O)[C@]1(Sc2ccc(C)cc2)C[C@H](O)[C@@H](NC(C)=O)[C@H]([C@H](O)[C@H](O)COS(=O)(=O)c2ccc(C)cc2)O1 ; COC(=O)[C@]1(Sc2ccc(C)cc2)C[C@H](O)[C@@H](NC(C)=O)[C@H]([C@H](O)[C@H](O)CN=[N+]=[N-])O1]

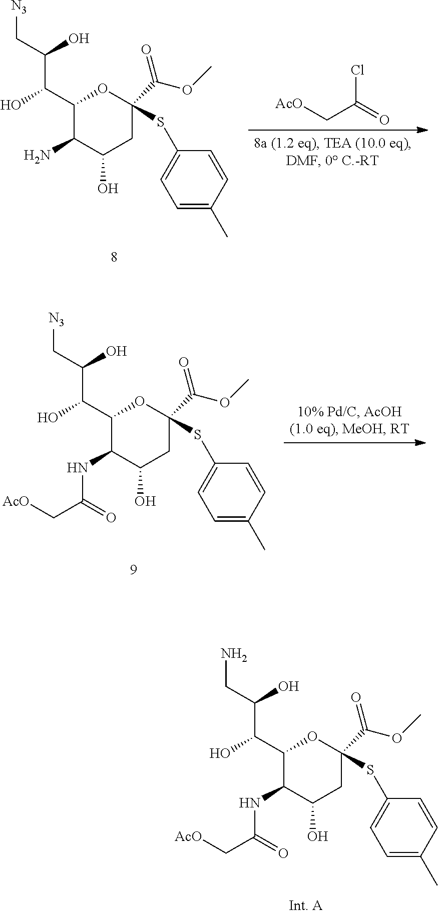 [SMILES: COC(=O)[C@]1(Sc2ccc(C)cc2)C[C@H](O)[C@@H](N)[C@H]([C@H](O)[C@H](O)CN=[N+]=[N-])O1 ; CC(=O)OCC(=O)Cl ; COC(=O)[C@]1(Sc2ccc(C)cc2)C[C@H](O)[C@@H](NC(=O)COC(C)=O)[C@H]([C@H](O)[C@H](O)CN=[N+]=[N-])O1 ; COC(=O)[C@]1(Sc2ccc(C)cc2)C[C@H](O)[C@@H](NC(=O)COC(C)=O)[C@H]([C@H](O)[C@H](O)CN)O1]

### Synthesis of methyl (2R,4S,5R,6R)-5-acetamido-2,4-dihydroxy-6-((1R,2R)-1,2,3-trihydroxypropyl)tetrahydro-2H-pyran-2-carboxylate (2)

To a stirred suspension of N-acetyl neuraminic acid (200.0 g, 646.6 mmol) in anhydrous methanol (2500 mL) was added trifluoroacetic acid (34.0 mL) at room temperature. The reaction mixture was stirred at 50° C. until the suspension became a clear solution. After completion the reaction mixture was filtered, and the filtrate was concentrated under reduced pressure. The residue obtained was triturated with diethyl ether, and the solid was collected by filtration to afford the desired product (2). Yield: 210.0 g, 98.0%; LCMS (ESI) m/z 324.2 [M+H]+.

### Synthesis of (1S,2R)-1-((2R,3R,4S,6S)-3-acetamido-4, 6-diacetoxy-6-(methoxycarbonyl)tetrahydro-2H-pyran-2-yl)propane-1,2,3-triyl triacetate (3)

In a 2000 mL round bottom flask methyl (2R,4S,5R,6R)-5-acetamido-2,4-dihydroxy-6-((1R,2R)-1,2,3-trihydroxypropyl)tetrahydro-2H-pyran-2-carboxylate (2, 210.0 g, 649.56 mmol) was dissolved with stirring in tetrahydrofuran (400 mL) under argon atmosphere. N,N-dimethylpyridin-4-amine (79.0 g, 649.56 mmol) was added at 0° C. followed by acetic anhydride (596 mL, 6495.6 mmol) dropwise over 30 minutes. The reaction mixture was allowed to warm up to room temperature overnight. The mixture was concentrated under reduced pressure and the residue obtained was partitioned between ethyl acetate and aq. saturated sodium bicarbonate. The organic layer was separated, dried filtered and concentrated. The residue obtained was triturated with diethyl ether, filtered and washed with diethyl ether to afford the desired product (3). Yield: 300.0 g, 86%; LCMS (ESI) m/z 534.2 [M+H]+.

### Synthesis of (1S,2R)-1-((2R,3R,4S,6R)-3-acetamido-4-acetoxy-6-(methoxycarbonyl)-6-(p-tolylthio)tetrahydro-2H-pyran-2-yl)propane-1,2,3-triyl triacetate (4)

To a mixture of (1S,2R)-1-((2R,3R,4S,6S)-3-acetamido-4,6-diacetoxy-6-(methoxycarbonyl)tetrahydro-2H-pyran-2-yl)propane-1,2,3-triyl triacetate (3, 300.0 g, 562.6 mmol), 4-methylbenzenethiol (843.4 mmol) and activated powdered 4 Å molecular sieves (400.0 g) in dichloromethane (3 L) at 0° C. was added boron trifluoride diethyl etherate (256.1 mL, 843.4 mmol) dropwise over 50 minutes. The mixture was stirred at room temperature until completion then filtered through celite. Aqueous saturated sodium bicarbonate was added under stirring and after 10 mins the organic layer was separated, washed with water, dried over anhydrous sodium sulfate, and concentrated. The crude residue obtained was purified by column chromatography using ethyl acetate and hexane as eluents to afford the desired product (4). Yield: 310.0 g, 91%; LCMS (ESI) m/z 598.32 [M+H]+.

### Synthesis of methyl (2R,4S,5R,6R)-5-acetamido-4-hydroxy-2-(p-tolylthio)-6-((1R,2R)-1,2,3-trihydroxypropyl)tetrahydro-2H-pyran-2-carboxylate (5)

To a stirred solution of (1S,2R)-1-((2R,3R,4S,6R)-3-acetamido-4-acetoxy-6-(methoxycarbonyl)-6-(p-tolylthio)tetrahydro-2H-pyran-2-yl)propane-1,2,3-triyl triacetate (4, 310.0 g, 519 mmol) in methanol (2400 mL) was added a solution of sodium methoxide (25% in MeOH) (11.5 mL, 51.9 mmol) dropwise at 0° C. The reaction mixture was stirred for 4 h at room temperature then cooled to 0° C. and quenched by adding DOWEX hydrogen form resin until pH 6. The mixture was filtered through celite and the filtrate was concentrated. The crude residue obtained was purified by column chromatography using methanol and ethyl acetate as eluents to afford the desired product (5). Yield: 200 g, 90%; LCMS (ESI) m/z 430.10 [M+H]+.

### Synthesis of methyl (2R,4S,5R,6R)-5-acetamido-6-((1R,2R)-1,2-dihydroxy-3-(tosyloxy)propyl)-4-hydroxy-2-(p-tolylthio)tetrahydro-2H-pyran-2-carboxylate (6)

To a stirred solution of methyl (2R,4S,5R,6R)-5-acetamido-4-hydroxy-2-(p-tolylthio)-6-((1R,2R)-1,2,3-trihydroxypropyl)tetrahydro-2H-pyran-2-carboxylate (5, 200.0 g, 465.6 mmol) in pyridine (1600 mL) and N,N-dimethylpyridin-4-amine (5.74 g, 45.6 mmol) was added 4-methylbenzene-1-sulfonyl chloride (132 g, 698.5 mmol) at 0° C. The reaction mixture was stirred at room temperature for 12 h, diluted with heptane and stirred for 5 more minutes. The solvent was removed and the process was repeated 4-5 times. The crude product was dried under vacuum then purified by column chromatography using ethyl acetate and hexane as eluents to afford the desired product (6). Yield: 110.0 g, 40.0%. LC-MS (ESI) m/z 584.05 [M+H]+.

### Synthesis of methyl (2R,4S,5R,6R)-5-acetamido-6-((1R,2R)-3-azido-1,2-dihydroxypropyl)-4-hydroxy-2-(p-tolylthio)tetrahydro-2H-pyran-2-carboxylate (7)

A mixture of methyl (2R,4S,5R,6R)-5-acetamido-6-((1R,2R)-1,2-dihydroxy-3-(tosyloxy)propyl)-4-hydroxy-2-(p-tolylthio)tetrahydro-2H-pyran-2-carboxylate (6, 110.0 g, 188.46 mmol) and sodium azide (122.6 g, 1884.8 mmol) was stirred in anhydrous N,N-dimethylformamide (1000.0 mL) at 100° C. for 6 h. The reaction mixture was cooled down to rt, diluted with ethyl acetate then washed three times with cold water. The organic layer was dried, filtered then concentrated. The crude residue obtained was purified by column chromatography using ethyl acetate and hexane as eluents to afford the desired product (7). Yield: 60.0 g, 70.05%; LCMS (ESI) m/z 453.10 [M−H]⁻.

### Synthesis of methyl (2R,4S,5R,6R)-5-amino-6-((1R,2R)-3-azido-1,2-dihydroxypropyl)-4-hydroxy-2-(p-tolylthio)tetrahydro-2H-pyran-2-carboxylate (8)

To a stirred solution of methyl (2R,4S,5R,6R)-5-acetamido-6-((1R,2R)-3-azido-1,2-dihydroxypropyl)-4-hydroxy-2-(p-tolylthio)tetrahydro-2H-pyran-2-carboxylate (7, 60.0 g, 132.0 mmol) in methanol (600.0 mL) was added methane sulfonic acid (43.0 mL, 660.7 mmol) dropwise at 0° C. The resulting reaction mixture was stirred at 80° C. for 30 h then concentrated. The residue was triturated with 10% ethyl acetate and heptane (4-5 times) to remove the excess methane sulfonic acid. The residue obtained was dried under vacuum to afford the desired product (8) which was used as such for the next step. Yield: 48.0 g; LCMS (ESI) m/z 413.57 [M+H]+.

### Synthesis of methyl (2R,4S,5R,6R)-5-(2-acetoxyacetamido)-6-((1R,2R)-3-azido-1,2-dihydroxypropyl)-4-hydroxy-2-(p-tolylthio)tetrahydro-2H-pyran-2-carboxylate (9)

To a stirred solution of methyl (2R,4S,5R,6R)-5-amino-6-((1R,2R)-3-azido-1,2-dihydroxypropyl)-4-hydroxy-2-(p-tolylthio)tetrahydro-2H-pyran-2-carboxylate (1, 15.0 g, 3.64 mmol) in dry N,N-dimethylformamide (140.0 mL) at 0° C. was added triethylamine (52.5 mL, 36.4 mmol), followed by a solution of 2-chloro-2-oxoethyl acetate (2, 5.32 mL, 4.36 mmol) in dry N,N-dimethylformamide (10.0 mL) dropwise at 0° C. The reaction mixture was allowed to warm up to room temperature over 2 h then concentrated. The crude residue obtained was purified by column chromatography using ethyl acetate and hexane as eluents to afford the desired product (9). Yield: 14.0 g, 75.0%; LCMS (ESI) m/z 513.15 [M+H]+; 1H NMR (400 MHz, methanol-d4) 8.26 (d, J=8.8 Hz, 1H), 7.45-7.42 (m, 2H), 7.21-7.17 (m, 2H), 4.62 (d, J=1.6 Hz, 2H), 4.57-4.54 (m, 1H), 3.99-3.31 (m, 11H), 2.67 (dd, J=4.8 Hz, & 13.6 Hz, 1H), 2.37-2.35 (m, 3H), 2.17-2.13 (m, 3H), 1.98-1.94 (m, 1H), 1.19-1.11 (m, 1H).

### Synthesis of methyl (2R,4S,5R,6R)-6-((1R,2R)-3-amino-1,2-dihydroxypropyl)-4-hydroxy-5-(2-hydroxyacetamido)-2-(p-tolylthio)tetrahydro-2H-pyran-2-carboxylate (Int. A)

To a stirred solution of methyl (2R,4S,5R,6R)-5-(2-acetoxyacetamido)-6-((1R,2R)-3-azido-1,2-dihydroxypropyl)-4-hydroxy-2-(p-tolylthio)tetrahydro-2H-pyran-2-carboxylate (9, 14.0 g, 27.34 mmol) in methanol (130 mL) and acetic acid (1.64 mL, 27.34 mmol) was added 10% Pd/C (14.0 g, 100% w/w) under argon at room temperature. The reaction mixture was stirred for 12 h under 1 atm of H₂ gas. The reaction mixture was filtered through celite and the filtrate was concentrated under reduced pressure to afford the desired product (Int. A) as a crude. Yield: 12.0 g; LCMS (ESI) m/z 487.15 [M+H]+. 1H NMR (400 MHz, methanol-d4): δ 7.45 (d, J=8.0 Hz, 2H), 7.24-7.19 (m, 2H), 4.57 (m, 2H), 4.04-3.98 (m, 1H), 3.97-3.66 (m, 5H), 3.53-3.42 (m, 4H), 3.33-3.39 (m, 1H), 2.98-2.86 (m, 1H), 2.87-2.82 (dd, J=4.8 Hz, 12.8 Hz, 1H), 2.38 (s, 3H), 2.19 (s, 3H), 1.92 (s, 3H), 1.89-1.86 (m, 1H), 1.99 (t, J=7.2 Hz, 3H).

### Intermediate B

### (2R,4S,5R,6S)-6-((S)-3-amino-2-hydroxypropyl)-2-(benzyloxy)-4-hydroxy-5-(2-hydroxyacetamido)tetrahydro-2H-pyran-2-carboxylic acid (Int. B)

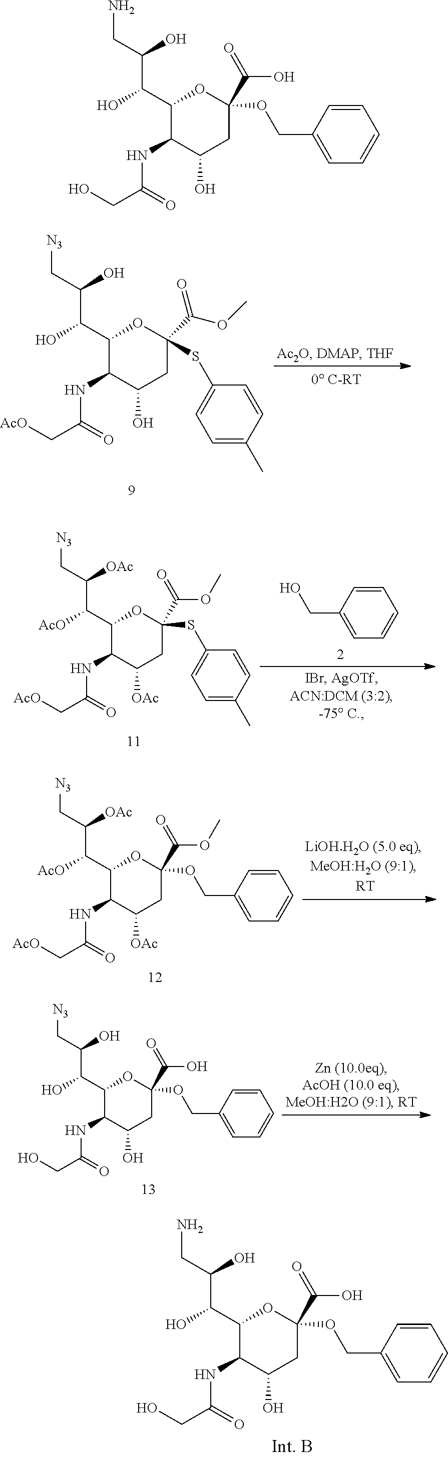

### Synthesis of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-azidopropane-1,2-diyl diacetate (11)

To a stirred solution of methyl (2R,4S,5R,6R)-5-(2-acetoxyacetamido)-6-((1R,2R)-3-azido-1,2-dihydroxypropyl)-4-hydroxy-2-(p-tolylthio)tetrahydro-2H-pyran-2-carboxylate (9, 35.0 g, 70.2 mmol) in dry tetrahydrofuran (360.0 mL) at 0° C. was added acetic anhydride (73.2 mL, 702 mmol), followed by N,N-dimethylpyridin-4-amine (8.58 g, 70.2 mmol) dropwise. The reaction mixture was stirred at 0° C. to room temperature for 16 h then concentrated. The crude residue obtained was diluted with aq. saturated sodium bicarbonate solution and extracted with ethyl acetate. The organic layer was washed with brine, dried, filtered, and concentrated. The crude residue obtained was purified by column chromatography using ethyl acetate and hexane as eluents to afford the desired product (11). Yield: 30.0 g, 70.0%; LCMS (ESI) m/z 638.90 [M+H]+. ¹H NMR (400 MHz, methanol-d4): δ 7.37-7.35 (m, 2H), 7.23-7.21 (m, 2H), 5.45-5.37 (m, 2H), 4.87-4.75 (m, 2H), 4.46-4.30 (m, 2H), 4.05-4.00 (m, 1H), 3.74-3.58 (m, 4H), 2.65-2.60 (dd, J=4.8 Hz, 1H), 2.37-2.35 (m, 3H), 2.14-1.95 (m, 13H).

### Synthesis of (1R,2R)-1-((2R,3R,4S,6R)-4-acetoxy-3-(2-acetoxyacetamido)-6-(benzyloxy)-6-(methoxycarbonyl)tetrahydro-2H-pyran-2-yl)-3-azidopropane-1,2-diyl diacetate (12)

A mixture of methyl (2R,4S,5R,6R)-4-(acetyloxy)-5-[2-(acetyloxy)acetamido]-6-[(1R,2R)-1,2-bis(acetyloxy)-3-azidopropyl]-2-[(4-methylphenyl)sulfanyl]oxane-2-carboxylate (11, 3.0 g, 4.70 mmol), benzyl alcohol (11, 2.54 g, 23.5 mmol), silver(1+) trifluoromethanesulfonate (2.41 g, 9.39 mmol) and activated 4 Å powdered molecular sieves (5.00 g) in anhydrous dichloromethane (60.0 mL) and anhydrous acetonitrile (90.0 mL) was stirred at room temperature for 1 h. The solution was cooled to −78° C., and a solution of iodine monobromide (1.46 g, 7.05 mmol) in dichloromethane (5.0 mL) was added dropwise. The reaction mixture was stirred at this temperature for 2 h then quenched by addition of triethylamine (3.0 mL). The cooling bath was removed, and the mixture was allowed to warm up to room temperature then filtered. The filtrate was washed with aq saturated sodium bicarbonate, then dried, filtered, and concentrated. The crude residue obtained was purified by column chromatography using ethyl acetate and heptane as eluents to afford the desired product (12). Yield: 2.50 g, 85%; LCMS m/z 620.91 [M−H]⁻.

### Synthesis of (2R,4S,5R,6R)-6-((1R,2R)-3-azido-1,2-dihydroxypropyl)-2-(benzyloxy)-4-hydroxy-5-(2-hydroxyacetamido)tetrahydro-2H-pyran-2-carboxylic acid (13)

To a stirred solution of methyl (2R,4S,5R,6R)-6-[(1R,2R)-1,2-diacetoxy-3-azidopropyl]-4-acetoxy-5-[(acetoxymethyl)carbonylamino]-2-(benzyloxy)tetrahydro-2H-pyran-2-carboxylate (12, 1.6 g, 2.57 mmol) in methanol (20.0 mL) and water (5.0 mL), was added lithium hydroxide monohydrate (0.647 g, 15.4 mmol) at 0° C. The reaction mixture was stirred at room temperature for 6 h. After completion, the reaction mixture was treated with an acid resin (Dowex 50, H⁺) to pH˜6. The suspension was filtered and washed with methanol. The filtrate was concentrated under reduced pressure. The crude material was purified by trituration with diethyl ether to afford the desired product (13). Yield: 1.00 g, 88%; LCMS m/z 441.10 [M+H]+.

### Synthesis of (2R,4S,5R,6S)-6-((S)-3-amino-2-hydroxypropyl)-2-(benzyloxy)-4-hydroxy-5-(2-hydroxyacetamido)tetrahydro-2H-pyran-2-carboxylic acid (Int. B)

To a stirred solution of (2R,4S,5R,6R)-6-[(1R,2R)-3-azido-1,2-dihydroxypropyl]-2-(benzyloxy)-4-hydroxy-5-[(hydroxymethyl)carbonylamino]tetrahydro-2H-pyran-2-carboxylic acid (13, 1.0 g, 2.95 mmol) in methanol (20.0 mL) and water (4.0 mL) was added Zn-powder (1.93 g, 29.50 mmol) and acetic acid (1.18 mL, 29.5 mmol) dropwise at 0° C. The reaction mixture was stirred at room temperature for 2 h then filtered through celite. The filtrate was concentrated, and the crude residue obtained was purified by column chromatography using water and acetonitrile as eluents to afford the desired product (Int. B). Yield: 0.90 g, 73.57%; LCMS m/z 398.17 [M+H]+;

### Intermediate C

### (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-ethynylphenyl)acetamido)propane-1,2-diyl diacetate (Int. C)

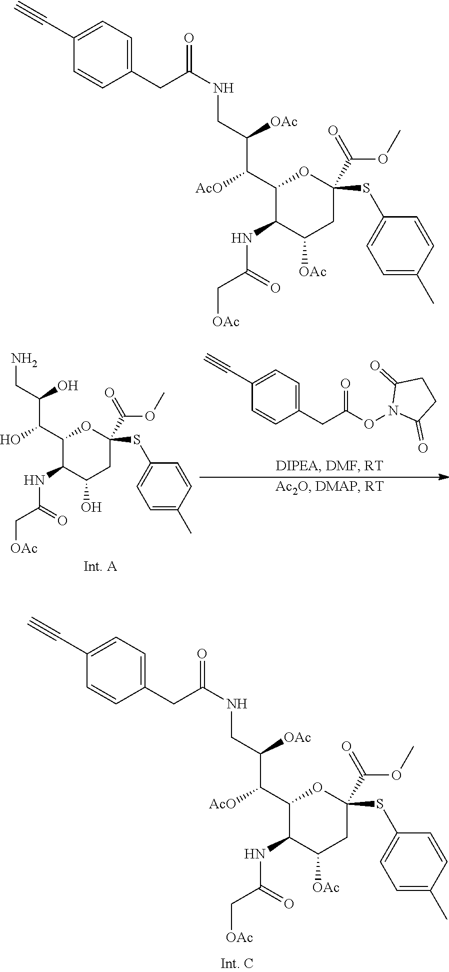

### Synthesis of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-ethynylphenyl)acetamido)propane-1,2-diyl diacetate (15)

To a stirred solution of methyl (2R,4S,5R,6R)-6-[(1R,2R)-3-amino-1,2-dihydroxypropyl]-5-[(acetoxymethyl)carbonylamino]-4-hydroxy-2-(p-tolylthio)tetrahydro-2H-pyran-2-carboxylate (Int. A, 0.5 g, 1.03 mmol) in N,N-dimethylformamide (5 mL) was added 1-[2-(p-ethynylphenyl)acetyloxy]-2,5-pyrrolidinedione (0.264 g, 1.03 mmol) and N,N-diisopropylethylamine (0.895 mL, 5.14 mmol) at room temperature. The resulting reaction mixture was stirred at room temperature for 4 h then acetic anhydride (1.61 mL, 15.9 mmol) and N,N-dimethylpyridin-4-amine (0.194 g, 1.59 mmol) were added at 0° C. The reaction mixture was stirred for 16 h at room temperature then concentrated. The crude residue obtained was taken up in ice water and extracted with ethyl acetate. The organic layer was washed with brine solution, dried, filtered and the filtrate was concentrated. The crude residue obtained was purified by column chromatography using ethyl acetate and heptane as eluents to afford the desired product (Int. C). Yield: 0.60 g, 49.98%; LCMS m/z 755.15 [M+H]+. ¹H NMR (400 MHz, MeOD-d4): δ 7.59-7.58 (m, 1H), 7.45-7.38 (m, 4H), 7.32-7.26 (m, 2H), 7.20-7.16 (m, 2H), 5.12-5.09 (m, 2H), 4.48-4.30 (m, 2H), 4.02-3.94 (m, 3H), 3.64-3.48 (m, 6H), 3.19-3.15 (m, 1H), 3.01-2.88 (m, 2H), 2.84-2.80 (dd, J=4.8 Hz, 12.6 Hz, 1H), 2.36 (s, 3H), 2.17-1.83 (m, 14H), 1.27-1.25 (m, 2H).

### Compound 1

### (2R,4S,5R,6R)-2-(benzyloxy)-6-((1R,2R)-3-(2-(4-ethynylphenyl)acetamido)-1,2-dihydroxypropyl)-4-hydroxy-5-(2-hydroxyacetamido)tetrahydro-2H-pyran-2-carboxylic acid (Cpd. 1)

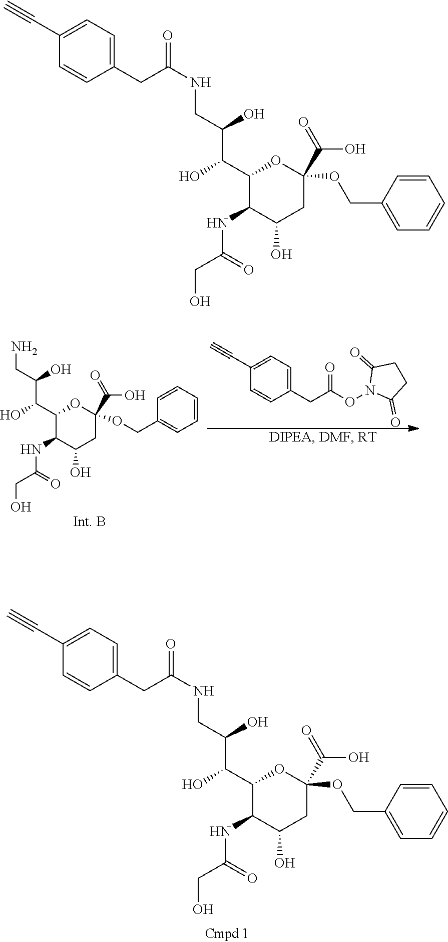 [SMILES: C#Cc1ccc(CC(=O)NC[C@@H](O)[C@@H](O)[C@@H]2O[C@@](OCc3ccccc3)(C(=O)O)C[C@H](O)[C@H]2NC(=O)CO)cc1 ; NC[C@@H](O)[C@@H](O)[C@@H]1O[C@@](OCc2ccccc2)(C(=O)O)C[C@H](O)[C@H]1NC(=O)CO ; C#Cc1ccc(CC(=O)ON2C(=O)CCC2=O)cc1 ; C#Cc1ccc(CC(=O)NC[C@@H](O)[C@@H](O)[C@@H]2O[C@](OCc3ccccc3)(C(=O)O)C[C@H](O)[C@H]2NC(=O)CO)cc1]

### Synthesis of (1R,2R)-1-((2R,3R,4S,6R)-4-acetoxy-3-(2-acetoxyacetamido)-6-(benzyloxy)-6-(methoxycarbonyl)tetrahydro-2H-pyran-2-yl)-3-azidopropane-1,2-diyl diacetate (Cpd. 1)

To a stirred solution of (2R,4S,5R,6R)-6-((1R,2R)-3-amino-1,2-dihydroxypropyl)-2-(benzyloxy)-4-hydroxy-5-(2-hydroxyacetamido)tetrahydro-2H-pyran-2-carboxylic acid (Int. B, 1.0 g, 2.41 mmol) and 2,5-dioxopyrrolidin-1-yl 2-(4-ethynylphenyl)acetate (6, 0.497 g, 1.93 mmol) in N,N-dimethylformamide (10.0 mL) was added N,N-diisopropylethylamine (2.10 mL, 12.05 mmol) at 0° C. and the reaction mixture was stirred at room temperature for 4 h then concentrated under reduced pressure. The crude residue obtained was purified by prep HPLC using water and acetonitrile (+0.1% TFA) as eluents to afford the desired product (Cpd. 1). Yield: 0.280 g, 21%; LCMS m/z 557.40 [M+H]+; 1H NMR (400 MHz, methanol-d4): δ 7.41-7.29 (m, 9H), 4.83 (d, J=11.2 Hz, 1H), 4.52 (d, J=11.2 Hz, 1H), 4.05 (s, 2H), 3.97-3.89 (m, 3H), 3.88-3.84 (m, 1H), 3.68 (dd, J=13.6 Hz & 2.8 Hz, 1H), 3.57 (s, 2H), 3.46 (s, 1H), 3.43 (dd, J=8.8 Hz & 1.2 Hz, 1H), 3.28-3.25 (m, 1H), 2.80 (dd, J=12.8 Hz & 4.0 Hz, 1H), 1.83 (t, J=12.4 Hz, 1H).

### Compound 2

### (2R,4S,5R,6R)-2-(benzyloxy)-6-((1R,2R)-1,2-dihydroxy-3-(2-(4-(1-(2-hydroxyethyl)-1H-1,2,3-triazol-4-yl)phenyl)acetamido)propyl)-4-hydroxy-5-(2-hydroxyacetamido)tetrahydro-2H-pyran-2-carboxylic acid (Cpd. 2)

### Synthesis of (2R,4S,5R,6R)-2-(benzyloxy)-6-((1R,2R)-1,2-dihydroxy-3-(2-(4-(1-(2-hydroxyethyl)-1H-1,2,3-triazol-4-yl)phenyl)acetamido)propyl)-4-hydroxy-5-(2-hydroxyacetamido)tetrahydro-2H-pyran-2-carboxylic acid (Cpd. 2)

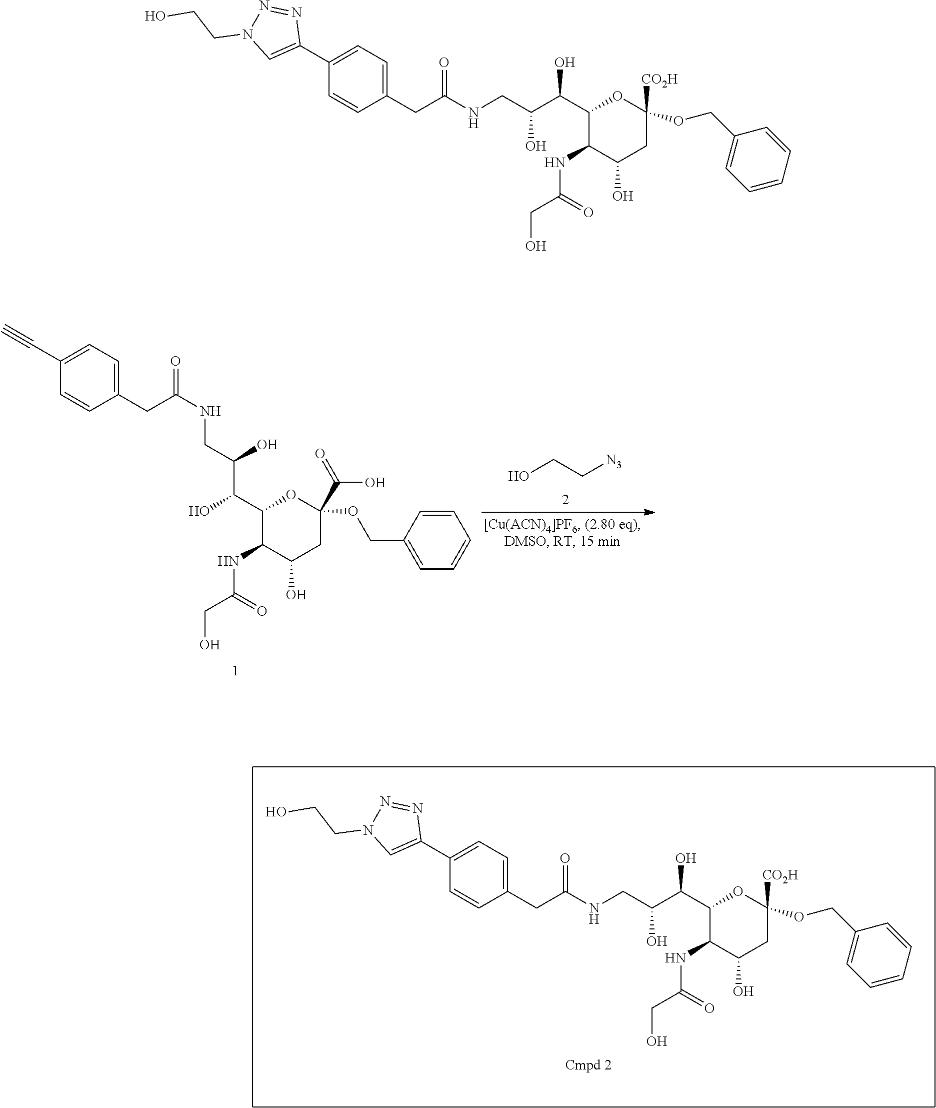 [SMILES: O=C(Cc1ccc(-c2cn(CCO)nn2)cc1)NC[C@@H](O)[C@@H](O)[C@@H]1O[C@@](OCc2ccccc2)(C(=O)O)C[C@H](O)[C@H]1NC(=O)CO ; C#Cc1ccc(CC(=O)NC[C@@H](O)[C@@H](O)[C@@H]2O[C@@](OCc3ccccc3)(C(=O)O)C[C@H](O)[C@H]2NC(=O)CO)cc1 ; [N-]=[N+]=NCCO ; O=C(Cc1ccc(-c2cn(CCO)nn2)cc1)NC[C@@H](O)[C@@H](O)[C@@H]1O[C@@](OCc2ccccc2)(C(=O)O)C[C@H](O)[C@H]1NC(=O)CO]

To a stirred solution of (2R,4S,5R,6R)-2-(benzyloxy)-6-((1R,2R)-3-(2-(4-ethynylphenyl)acetamido)-1,2-dihydroxypropyl)-4-hydroxy-5-(2-hydroxyacetamido)tetrahydro-2H-pyran-2-carboxylic acid (Cpd. 1, 0.030 g, 0.054 mmol) and 2-azidoethan-1-ol (0.0051 g, 0.059 mmol) in dimethyl sulfoxide (0.5 mL) was added tetrakis(acetonitrile)copper(I) hexafluorophosphate (0.056 g, 0.151 mmol) and the reaction mixture was stirred at room temperature for 30 min. Acetic acid (0.3 mL) was added to the mixture and the resulting solution was purified by prep HPLC using acetonitrile and water (+0.1% TFA) as eluents to afford the desired product (Cpd. 2). Yield: 0.033 g, 98%; LCMS (ESI) m/z 644.35 [M+H]⁺; ¹H NMR (400 MHz, methanol-d4) δ 8.27 (s, 1H), 7.76 (d, J=8.0 Hz, 2H), 7.39 (d, J=80 Hz, 2H), 7.32-7.24 (m, 5H), 4.81 (d, J=11.2 Hz, 1H), 4.54-4.46 (m, 3H), 4.01 (s, 2H), 3.99-3.75 (m, 6H), 3.68 (dd, J=13.6 Hz & 2.8 Hz, 1H), 3.58 (s, 2H), 3.41 (dd, J=8.8 Hz & 1.6 Hz, 1H), 3.30-3.25 (m, 1H), 2.78 (dd, J=12.4 Hz, 2.0 Hz, 1H), 1.83-1.77 (m, 1H).

### Compound 3

### (2R,4S,5R,6R)-6-((1R,2R)-3-(2-(4-ethynylphenyl)acetamido)-1,2-dihydroxypropyl)-4-hydroxy-5-(2-hydroxyacetamido)-2-((4-hydroxybenzyl)oxy)tetrahydro-2H-pyran-2-carboxylic acid (Cpd. 3)

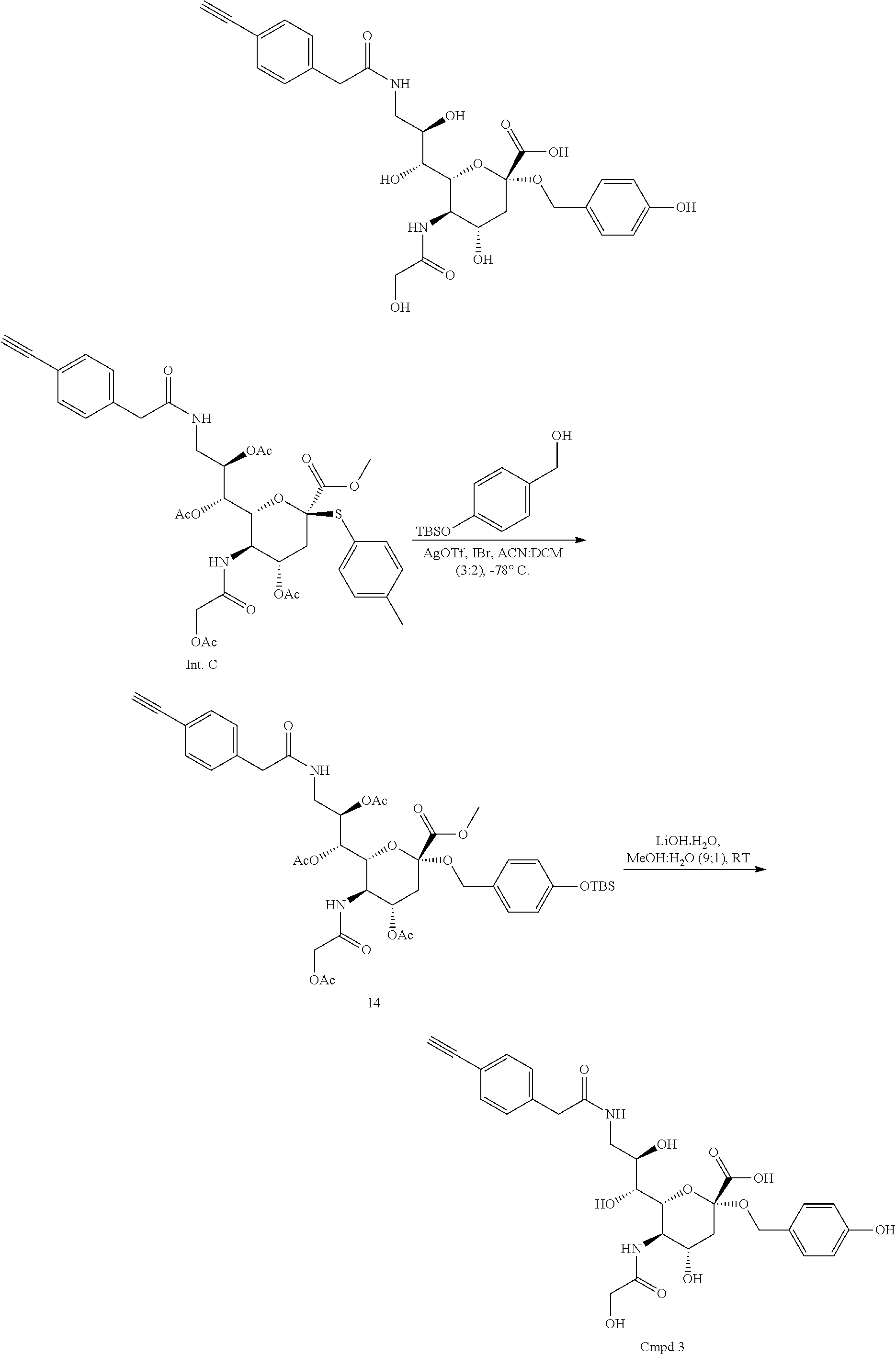

### Synthesis of (1R,2R)-1-((2R,3R,4S,6R)-4-acetoxy-3-(2-acetoxyacetamido)-6-((4-((tert-butyldimethylsilyl)oxy)benzyl)oxy)-6-(methoxycarbonyl)tetrahydro-2H-pyran-2-yl)-3-(2-(4-ethynylphenyl)acetamido)propane-1,2-diyl diacetate (14)

To a stirred solution of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-ethynylphenyl)acetamido)propane-1,2-diyl diacetate (Int. C, 0.350 g, 0.464 mmol) and (4-((tert-butyldimethylsilyl)oxy)phenyl)methanol (0.553 g, 2.32 mmol), silver(1+) trifluoromethanesulfonate (0.238 g, 0.927 mmol)) and activated 4 Å powdered molecular sieves (1.00 g) in anhydrous dichloromethane (12.0 mL) and anhydrous acetonitrile (7.0 mL) was stirred at room temperature for 1 h under nitrogen atmosphere. The solution was cooled to −78° C., and a solution of iodine monobromide (0.144 g, 0.696 mmol) in dichloromethane (1.0 mL) was added dropwise. The mixture was stirred at the same temperature for 2 h. The reaction mixture was quenched with triethylamine (1.0 mL) and allowed to warm up to room temperature. The reaction mixture was filtered, and the filtrate was washed with aq. sat. solution of sodium bicarbonate. The organic phase was dried, filtered, and concentrated. The crude residue obtained was purified by column chromatography using ethyl acetate and heptane as eluents to afford the desired product (14). Yield: 0.250 g, 62%; LCMS m/z 868.95 [M+H]⁺.

### Synthesis of (2R,4S,5R,6R)-6-((1R,2R)-3-(2-(4-ethynylphenyl)acetamido)-1,2-dihydroxypropyl)-4-hydroxy-5-(2-hydroxyacetamido)-2-((4-hydroxybenzyl)oxy)tetrahydro-2H-pyran-2-carboxylic acid (Cpd. 3)

To a stirred solution of (1R,2R)-1-((2R,3R,4S,6R)-4-acetoxy-3-(2-acetoxyacetamido)-6-((4-((tert-butyldimethylsilyl)oxy)benzyl)oxy)-6-(methoxycarbonyl)tetrahydro-2H-pyran-2-yl)-3-(2-(4-ethynylphenyl)acetamido)propane-1,2-diyl diacetate (14, 0.25 g, 0.287 mmol) in methanol (3.0 ml) was added a solution of lithium hydroxide monohydrate (0.072 g, 1.722 mmol) in water (0.50 ml). The reaction mixture was stirred at room temperature for 6 h. After completion, the reaction mixture was treated with acidic resin (Dowex 50, H⁺) to pH˜6 and the suspension was filtered. The filtrate was concentrated under reduced pressure and the crude residue obtained was purified by prep HPLC using acetonitrile and water (+0.1% TFA) as eluents to afford the desired product (Cpd. 3). Yield: 0.014 g; 9%; LCMS (ESI) m/z 573.40 [M+H]⁺. ¹H NMR (400 MHz, DMSO-d₆): δ 9.40 (s, 1H), 8.00 (t, J=5.2 Hz, 1H), 7.86 (d, J=8.0 Hz, 1H), 7.36 (d, J=10.4 Hz, 2H), 7.25 (d, J=8.4 Hz, 2H), 7.08 (d, J=8.4 Hz, 2H), 6.71 (d, J=8.4 Hz, 2H), 5.53 (t, J=5.6 Hz, 1H), 4.96 (d, J=5.6 Hz, 1H), 4.73 (d, J=4.8 Hz, 1H), 4.57 (d, J=10.8 Hz, 1H), 4.27 (d, J=11.2 Hz, 1H), 4.11 (s, 1H), 3.92-3.82 (s, 1H), 3.75-3.72 (m, 1H), 3.64-3.52 (m, 3H), 3.46 (s, 1H), 3.24-3.21 (m, 1H), 2.99-2.93 (m, 1H), 1.55 (t, J=12.0 Hz, 1H).

### Compound 4

### (2R,4S,5R,6R)-2-(benzyloxy)-6-((1R,2R)-1,2-dihydroxy-3-(2-(4-(prop-2-yn-1-yloxy)phenyl)acetamido)propyl)-4-hydroxy-5-(2-hydroxyacetamido)tetrahydro-2H-pyran-2-carboxylic acid (Cpd. 4)

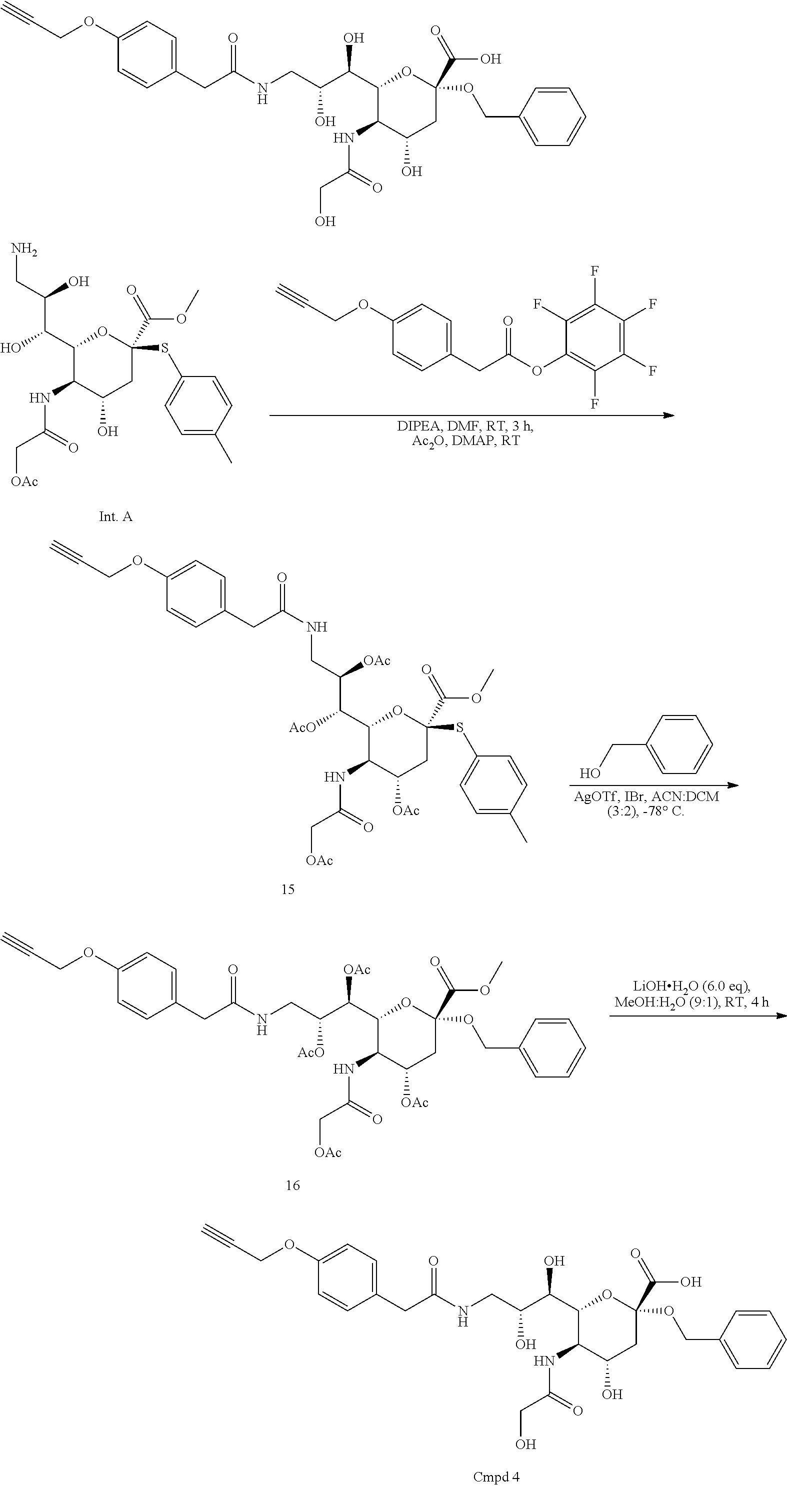

### Synthesis of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-(prop-2-yn-1-yloxy)phenyl)acetamido)propane-1,2-diyl diacetate (15)

To a stirred solution of methyl (2R,4S,5R,6R)-6-((1R,2R)-3-amino-1,2-dihydroxypropyl)-4-hydroxy-5-(2-hydroxyacetamido)-2-(p-tolylthio)tetrahydro-2H-pyran-2-carboxylate (Int. A, 0.50 g, 1.12 mmol) and perfluorophenyl 2-(4-(prop-2-yn-1-yloxy)phenyl)acetate (0.321 g, 0.9 mmol) in N,N-dimethylformamide (5.0 mL) was added N,N-diisopropylethylamine (0.97 mL, 5.62 mmol) at 0° C. and the reaction mixture was stirred at room temperature for 4 h. Acetic anhydride (1.06 mL, 11.25 mmol) and 4-dimethylaminopyridine (0.027 g, 0.224 mmol) were added at 0° C. and the mixture was stirred at room temperature for 12 h. The reaction mixture was diluted with water and ethyl acetate. The organic layer was separated, dried, filtered, and concentrated. The crude residue obtained was purified by column chromatography using ethyl acetate and heptane as eluents to afford the desired product (15). Yield: 0.350 g, 40%; LCMS m/z 685.25 [M+H]⁺.

### Synthesis of (1R,2R)-1-((2R,3R,4S,6R)-4-acetoxy-3-(2-acetoxyacetamido)-6-(benzyloxy)-6-(methoxycarbonyl)tetrahydro-2H-pyran-2-yl)-3-(2-(4-(prop-2-yn-1-yloxy)phenyl)acetamido)propane-1,2-diyl diacetate (16)

A mixture of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-(prop-2-yn-1-yloxy)phenyl)acetatedo)propane-1,2-diyl iacetate (15, 0.40 g, 0.510 mmol), benzyl alcohol (0.276 g, 2.55 mmol), silver(1+) trifluoromethanesulfonate (0.262 g, 1.02 mmol)) and activated 4 A powdered molecular sieves (1.00 g) in dichloromethane (15.0 mL) and acetonitrile (9.0 mL) was stirred at room temperature for 1 h. The solution was cooled to −78° C., then a solution of iodine monobromide (0.158 g, 0.764 mmol) in dichloromethane (1.0 mL) was added dropwise and the mixture was stirred at the same temperature for 2 h. The reaction mixture was quenched with triethylamine (1.0 mL), warmed up to room temperature then filtered. The filtrate was washed with aq. sat. NaHCO₃, dried, filtered then concentrated. The crude residue obtained was purified by column chromatography using ethyl acetate and heptane as eluents to afford the desired product (16). Yield: 0.20 g, 51.0%; LCMS m/z 769.30 [M+H]⁺.

### Synthesis of (2R,4S,5R,6R)-2-(benzyloxy)-6-((1R,2R)-1,2-dihydroxy-3-(2-(4-(prop-2-yn-1-yloxy)phenyl)acetamido)propyl)-4-hydroxy-5-(2-hydroxyacetamido)tetrahydro-2H-pyran-2-carboxylic acid (Cpd. 4)

To a stirred solution of (1R,2R)-1-((2R,3R,4S,6R)-4-acetoxy-3-(2-acetoxyacetamido)-6-(benzyloxy)-6-(methoxycarbonyl)tetrahydro-2H-pyran-2-yl)-3-(2-(4-(prop-2-yn-1-yloxy)phenyl)acetamido)propane-1,2-diyl diacetate (22, 0.20 g, 0.260 mmol) in methanol (2.0 mL) and water (0.20 mL) was added lithium hydroxide monohydrate (0.21 g, 1.35 mmol) at 0° C. The reaction mixture was stirred at room temperature for 6 h. The reaction mixture was treated with acidic resin (Dowex 50, H⁺) to pH˜6 and the suspension was filtered. The filtrate was concentrated, and the crude residue obtained was purified by prep HPLC using water and acetonitrile (+0.1% TFA) as eluents to afford the desired product (Cpd. 4). Yield: 0.045 g, 29%; LCMS m/z 587.35 [M+H]⁺. ¹H NMR (400 MHz, methanol-d4): δ 7.93 (d, J=7.6 Hz, 1H), 7.32-7.26 (m, 5H), 7.21 (d, J=8.8 Hz, 2H), 6.92-6.89 (m, 2H), 4.80 (d, J=11.2 Hz, 1H), 4.65 (d, J=2.4 Hz, 2H), 4.48 (d, J=11.2 Hz, 1H), 4.02 (s, 1H), 3.93-3.81 (m, 3H), 3.81-3.76 (m, 1H), 3.65 (dd, J=13.6 Hz & 2.8 Hz, 1H), 3.47 (s, 2H), 3.39 (dd, J=8.8 Hz & 1.2 Hz, 1H), 3.27-3.22 (m, 1H), 2.90 (t, J=2.4 Hz, 1H), 2.78 (dd, J=12.4 Hz & 4.0 Hz, 1H), 1.79 (t, J=12.0 Hz, 1H).

### Compound 5

### (2R,4S,5R,6R)-2-(benzyloxy)-6-((1R,2R)-1,2-dihydroxy-3-(2-(4-((1-(2-hydroxyethyl)-1H-1,2,3-triazol-4-yl)methoxy)phenyl)acetamido)propyl)-4-hydroxy-5-(2-hydroxyacetamido)tetrahydro-2H-pyran-2-carboxylic acid (Cpd. 5)

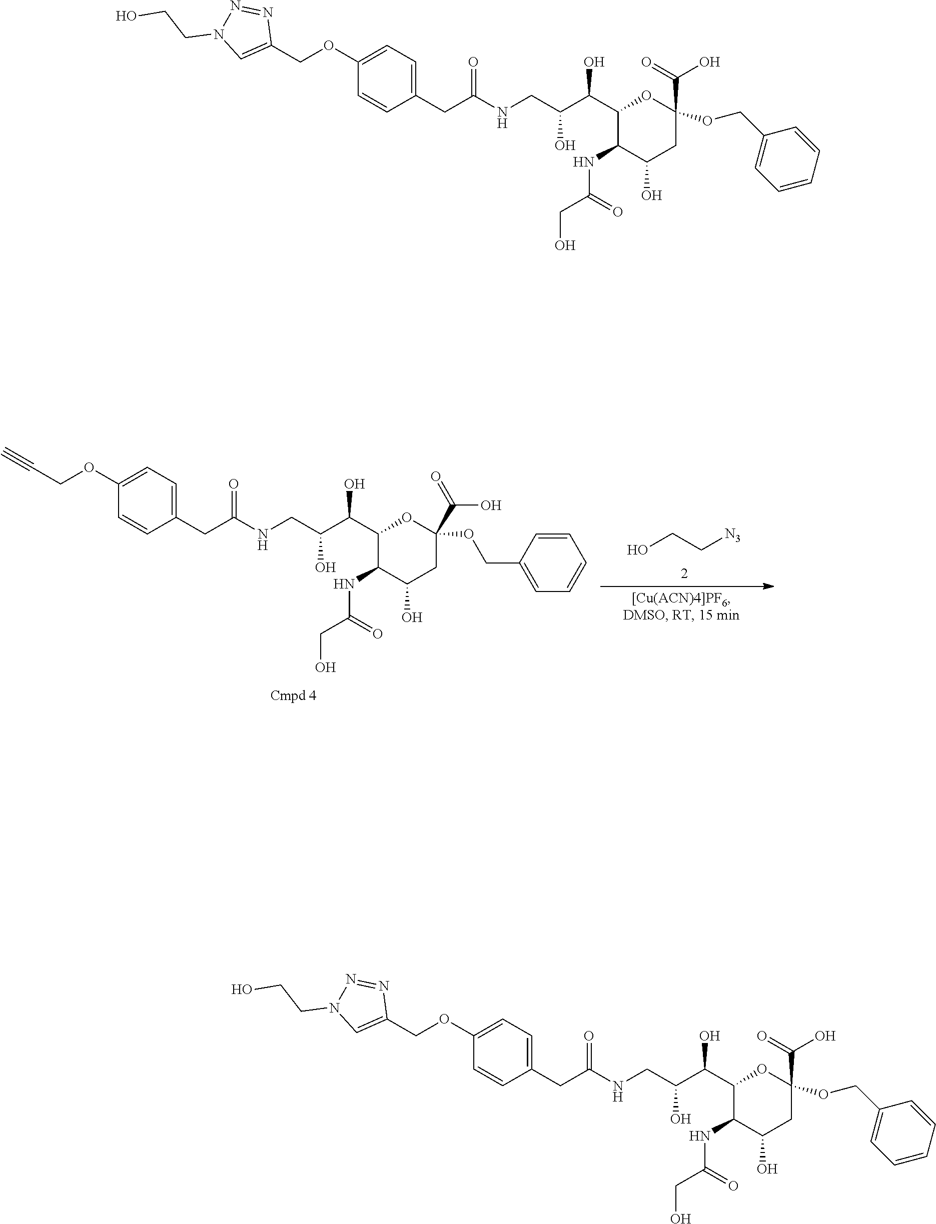 [SMILES: O=C(Cc1ccc(OCc2cn(CCO)nn2)cc1)NC[C@@H](O)[C@@H](O)[C@@H]1O[C@@](OCc2ccccc2)(C(=O)O)C[C@H](O)[C@H]1NC(=O)CO ; C#CCOc1ccc(CC(=O)NC[C@@H](O)[C@@H](O)[C@@H]2O[C@@](OCc3ccccc3)(C(=O)O)C[C@H](O)[C@H]2NC(=O)CO)cc1 ; [N-]=[N+]=NCCO ; O=C(Cc1ccc(OCc2cn(CCO)nn2)cc1)NC[C@@H](O)[C@@H](O)[C@@H]1O[C@@](OCc2ccccc2)(C(=O)O)C[C@H](O)[C@H]1NC(=O)CO]

### Synthesis of (2R,4S,5R,6R)-2-(benzyloxy)-6-((1R,2R)-1,2-dihydroxy-3-(2-(4-((1-(2-hydroxyethyl)-1H-1,2,3-triazol-4-yl)methoxy)phenyl)acetamido)propyl)-4-hydroxy-5-(2-hydroxyacetamido)tetrahydro-2H-pyran-2-carboxylic acid (Cpd. 5)

To a stirred solution of (2R,4S,5R,6R)-2-(benzyloxy)-6-((1R,2R)-3-(2-(4-ethynylphenyl)acetamido)-1,2-dihydroxypropyl)-4-hydroxy-5-(2-hydroxyacetamido)tetrahydro-2H-pyran-2-carboxylic acid (Cpd. 4, 0.025 g, 0.0426 mmol) and 2-azidoethan-1-ol (0.004 g, 0.0469 mmol) in dimethyl sulfoxide (0.5 mL) was added tetrakis(acetonitrile)copper(I) hexafluorophosphate (0.044 g, 0.119 mmol) and the reaction mixture was stirred at room temperature for 30 min. The solution was diluted with acetonitrile, filtered, and purified by prep HPLC using water and acetonitrile as eluents (+0.1% TFA) to afford the desired product (Cpd. 5). Yield: 0.006 g, 21%; LCMS (ESI) m/z 674.35 [M+H]⁺; ¹H NMR (400 MHz, methanol-d₄): δ 8.02 (s, 1H), 7.33-7.23 (m, 5H), 7.21 (d, J=8.4 Hz, 2H), 6.93 (d, J=8.4 Hz, 2H), 5.10 (s, 2H), 4.79 (d, J=11.2 Hz, 1H), 4.50-4.47 (m, 3H), 4.01 (s, 2H), 3.93-3.75 (m, 6H), 3.63 (dd, J=14.0 Hz & 3.6 Hz, 1H), 3.46 (s, 2H), 3.38 (d, J=8.0 Hz, 1H), 3.30-3.22 (m, 1H), 2.81-2.78 (m, 1H), 1.77 (t, J=12 Hz, 1H).

### Compound 6

### (2R,4S,5R,6R)-2-(benzyloxy)-6-((1R,2R)-3-(2-(4-(deca-1,9-diyn-1-yl)phenyl)acetamido)-1,2-dihydroxypropyl)-4-hydroxy-5-(2-hydroxyacetamido)tetrahydro-2H-pyran-2-carboxylic acid (Cpd. 6)

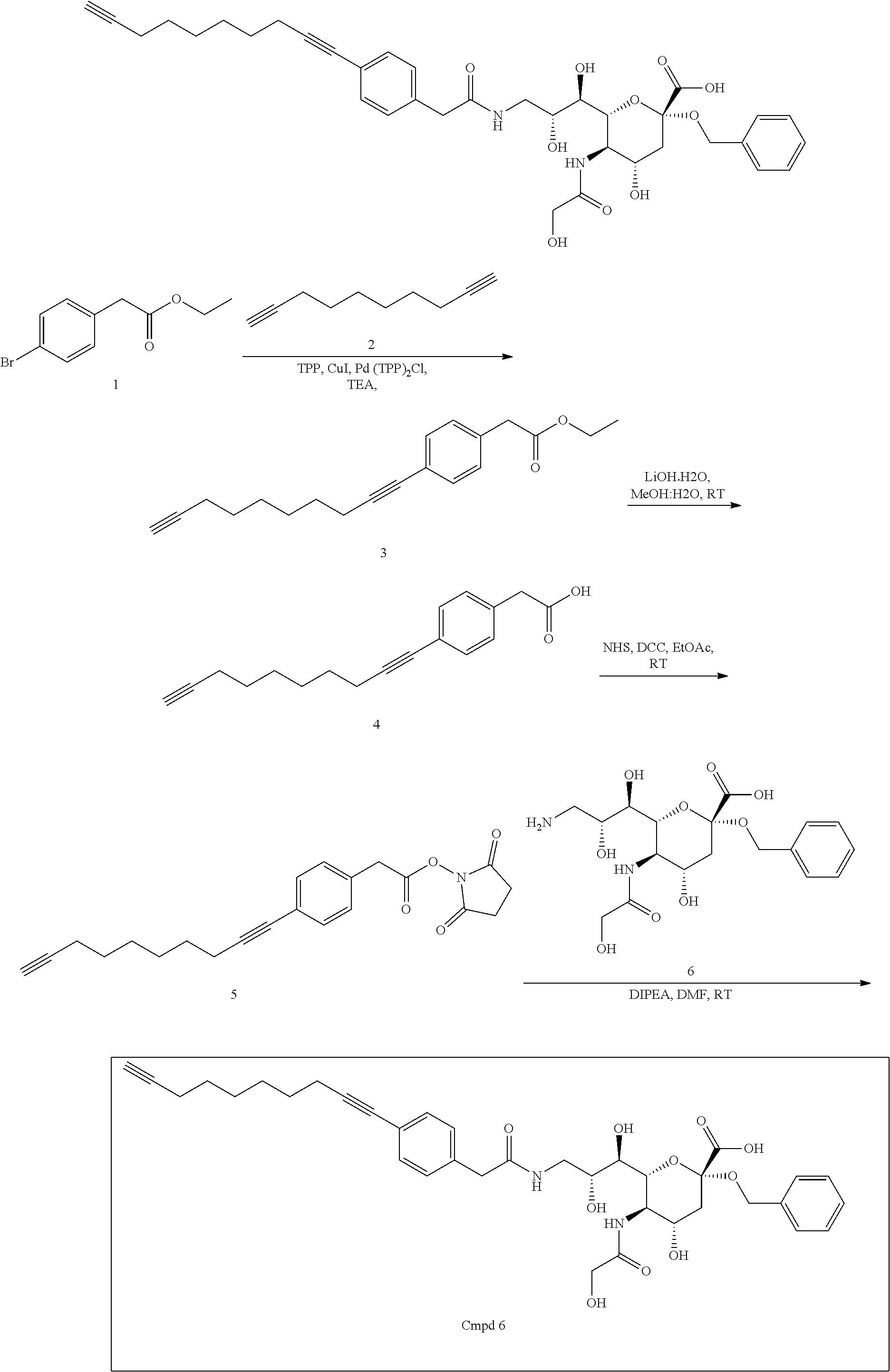

### Synthesis of ethyl 2-(4-(deca-1,9-diyn-1-yl)phenyl)acetate (3)

A degassed solution of ethyl 2-(4-bromophenyl)acetate (1, 1.0 g, 4.11 mmol), deca-1,9-diyne (2, 0.552 g, 4.11 mmol), copper iodide (0.157 g, 0.823 mmol), triphenylphosphine (0.539 g, 2.06 mmol) and palladium(2+) bis(triphenylphosphane) dichloride (0.577 g, 0.823 mmol) in triethyl amine (5.0 mL), was heated at 65° C. for 6 h. After completion the reaction mixture was quenched by slow addition of 1N HCl at 0° C., then extracted with ethyl acetate. The combined organic layers were concentrated under reduced pressure and the crude residue obtained was purified by silica-gel column chromatography using ethyl acetate and heptane as eluents to afford the desired product. Yield: 0.60 g, 49%; LCMS m/z 295.15 [M−H]−.

### Synthesis of 2-(4-(deca-1,9-diyn-1-yl)phenyl)acetic acid (4)

To a stirred solution of ethyl 2-(4-(deca-1,9-diyn-1-yl)phenyl)acetate (3, 0.350 g, 1.18 mmol) in ethanol (5.0 mL) was added a solution of lithium hydroxide monohydrate (0.149 g, 3.540 mmol) in water (1.0 mL). The reaction mixture was stirred at room temperature for 5 h then concentrated. The residue obtained was taken up in 1N HCl solution and the suspension was filtered, washed with water then diethyl ether to afford the desired product. Yield: 0.30 g, 95%; LCMS (ESI) m/z 267.20 [M−H]−.

### Synthesis of 2,5-dioxopyrrolidin-1-yl 2-(4-(deca-1,9-diyn-1-yl)phenyl)acetate (5)

To a stiffed solution of 2-(4-(deca-1,9-diyn-1-yl)phenyl) acetic acid (4, 0.30 g, 1.12 mmol) in ethyl acetate (3.0 mL), 1-hydroxypyrrolidine-2,5-dione (0.129 g, 1.12 mmol) and N,N′-dicyclohexylmethanediimine (0.231 g, 1.12 mmol) was added at 0° C. and the reaction mixture was stirred at room temperature for 4 h. After completion, the reaction mixture was concentrated and the residue obtained was dissolved in ethyl acetate, filtered and washed with ethyl acetate to afford the desired product. Yield: 0.250 g, 61%; LCMS (ESI) m/z 363.95 [M−H]−.

### Synthesis of (2R,4S,5R,6R)-2-(benzyloxy)-6-((1R,2R)-3-(2-(4-(deca-1,9-diyn-1-yl)phenyl)acetamido)-1,2-dihydroxypropyl)-4-hydroxy-5-(2-hydroxyacetamido)tetrahydro-2H-pyran-2-carboxylic acid (Cpd. 6)

To a stirred solution of (2R,4S,5R,6R)-6-((1R,2R)-3-amino-1,2-dihydroxypropyl)-2-(benzyloxy)-4-hydroxy-5-(2-hydroxyacetamido)tetrahydro-2H-pyran-2-carboxylic acid (6, 0.250 g, 0.603 mmol) and 2,5-dioxopyrrolidin-1-yl 2-(4-(deca-1,9-diyn-1-yl)phenyl)acetate (5, 0.176 g, 0.483 mmol) in N,N-dimethylformamide (3.0 mL) was added ethylbis(propan-2-yl)amine (0.52 mL, 3.02 mmol) at 0° C. and the reaction mixture was stirred at room temperature for 4 h. The reaction mixture was concentrated under reduced pressure and the crude residue obtained was purified by prep HPLC using acetonitrile and water (+0.1% TFA) as eluents to afford the desired product (Cpd. 6). Yield: 0.065 g; 16%; LCMS (ESI) m/z 665.50 [M+H]+. 1H NMR (400 MHz, MeOD-d4): δ 7.94 (d, J=8.0 Hz, 1H), 7.32-7.21 (m, 9H), 4.80 (d, J=11.2 Hz, 1H), 4.48 (d, J=11.2 Hz, 1H), 4.02 (s, 2H), 3.94-3.77 (m, 4H), 3.66 (dd, J=13.6 Hz & 3.2 Hz, 1H), 3.52 (s, 2H), 3.40 (dd, J=8.8 Hz & 1.6 Hz, 1H), 3.27-3.25 (m, 1H), 2.78 (dd, J=12.4 Hz & 4.0 Hz, 1H), 2.39 (t, J=6.8 Hz, 2H), 2.20-2.16 (m, 3H), 1.80 (t, J=12.4 Hz, 1H), 1.61-1.46 (m, 8H).

### Compound 7

### (2R,4S,5R,6R)-2-(benzyloxy)-6-((1R,2R)-3-(2-(4-(dec-9-yn-1-yloxy)phenyl)acetamido)-1,2-dihydroxypropyl)-4-hydroxy-5-(2-hydroxyacetamido)tetrahydro-2H-pyran-2-carboxylic acid (Cpd. 7)

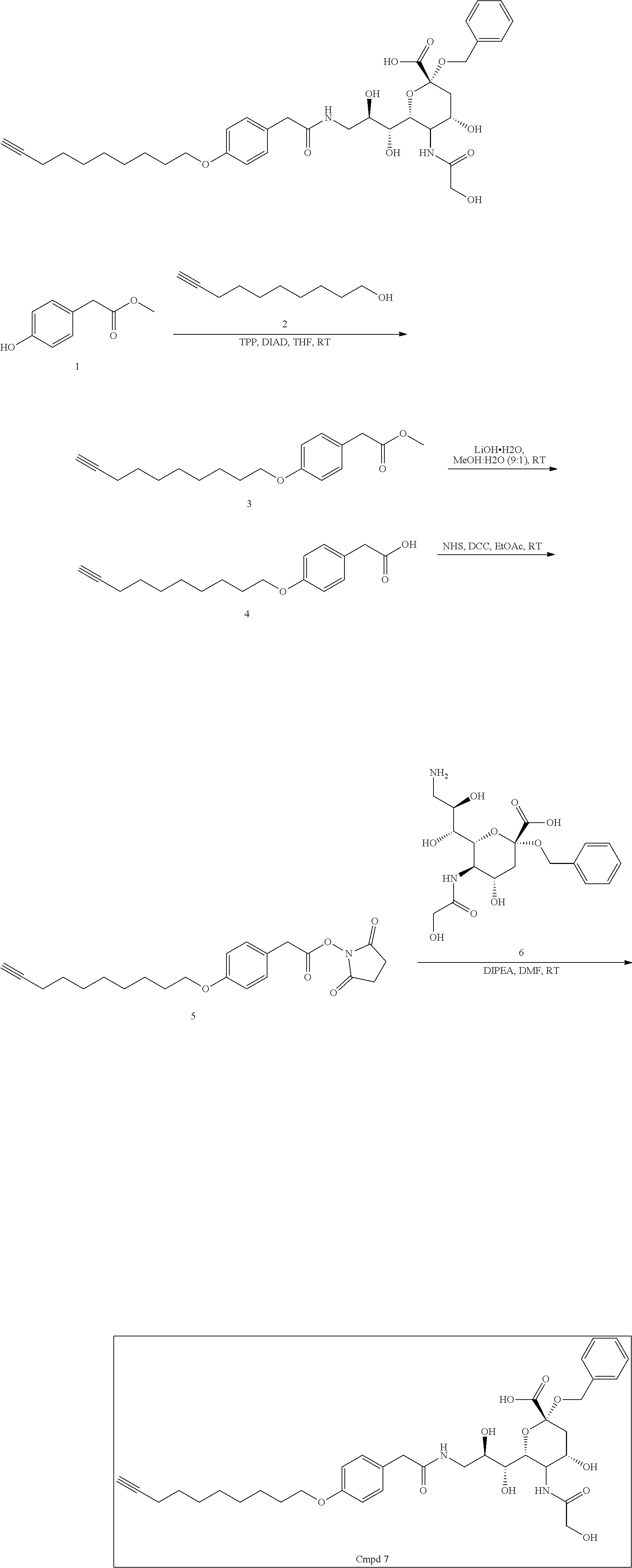

### Synthesis of methyl 2-(4-(dec-9-yn-1-yloxy)phenyl)acetate (3)

A solution of methyl 2-(4-hydroxyphenyl)acetate (1, 1.0 g, 6.02 mmol) in tetrahydrofuran (10.0 mL), triphenylphosphine (3.16 g, 12.04 mmol), 2-(2-(prop-2-yn-1-yloxy)ethoxy)ethan-1-ol (1.86 g, 12.04 mmol) and (E)-N-[(ethoxycarbonyl)imino]ethoxyformamide (2.38 mL, 15.00 mmol) was added at 0° C. and stirred at room temperature for 12 h. After completion the reaction mixture was diluted with water and extracted with ethyl acetate. The combined organic layers were dried, filtered and concentrated under reduced pressure. The crude residue obtained was purified by silica-gel column chromatography using ethyl acetate and heptane as eluents, to afford the desired product. Yield: 0.75 g, 41%; LCMS m/z 320.15 [M+H₂O]+.

### Synthesis of 2-(4-(dec-9-yn-1-yloxy)phenyl)acetic acid (4)

To a stirred solution of methyl 2-(4-(dec-9-yn-1-yloxy)phenyl)acetate (3, 0.70 g, 2.31 mmol) in methanol (8.0 mL) was added a solution of lithium hydroxide monohydrate (0.166 g, 6.94 mmol) in water (1.0 mL). The reaction mixture was stirred at room temperature for 5 h. After completion the reaction mixture was concentrated under reduced pressure and the mixture was treated with 1N HCl solution. The suspension was filtered and washed with water and diethyl ether to afford the desired product. Yield: 0.50 g, 75%; LCMS (ESI) m/z 287.10 [M−H]−.

### Synthesis of 2,5-dioxopyrrolidin-1-yl 2-(4-(dec-9-yn-1-yloxy)phenyl)acetate (5)

To a stirred solution of 2-(4-(dec-9-yn-1-yloxy)phenyl)acetic acid (4, 0.50 g, 1.73 mmol) in ethyl acetate (5.0 mL), 1-hydroxypyrrolidine-2,5-dione (0.199 g, 1.73 mmol) and N,N′-dicyclohexylmethanediimine (0.356 g, 1.73 mmol) were added at 0° C. and the reaction mixture was stirred at room temperature for 4 h. After completion the reaction mixture was concentrated and the crude residue obtained was dissolved in ethyl acetate, filtered, then washed with ethyl acetate to afford the desired product. Yield: 0.420 g, 63%; LCMS (ESI) m/z 403.10 [M+H2O]+.

### Synthesis of (2R,4S,5R,6R)-2-(benzyloxy)-6-((1R,2R)-3-(2-(4-(dec-9-yn-1-yloxy)phenyl)acetamido)-1,2-dihydroxypropyl)-4-hydroxy-5-(2-hydroxyacetamido)tetrahydro-2H-pyran-2-carboxylic acid (Cpd. 7)

To a stirred solution of (2R,4S,5R,6R)-6-((1R,2R)-3-amino-1,2-dihydroxypropyl)-2-(benzyloxy)-4-hydroxy-5-(2-hydroxyacetamido)tetrahydro-2H-pyran-2-carboxylic acid (6, 0.250 g, 0.603 mmol) and 2,5-dioxopyrrolidin-1-yl 2-(4-(dec-9-yn-1-yloxy)phenyl)acetate (5, 0.186 g, 0.482 mmol) in N,N-dimethylformamide (3.0 mL) was added ethylbis(propan-2-yl)amine (0.52 mL, 3.02 mmol) at 0° C. and the reaction mixture was stirred at room temperature for 4 h. After completion the reaction mixture was concentrated. The crude residue obtained was purified by prep HPLC using acetonitrile and water (+0.1% TFA) as eluents to afford the desired product (Cpd. 7). Yield: 0.088 g; 21%; LCMS (ESI) m/z 685.50 [M+H]+; 1H NMR (400 MHz, MeOD-d4): δ 7.93 (d, J=8.4 Hz, 1H), 7.31 (d, J=4.4 Hz, 4H), 7.29-7.25 (m, 1H), 7.18 (d, J=8.8 Hz, 2H), 6.82 (dt, J=9.6 Hz & 2.8 Hz, 2H), 4.79 (d, J=11.6 Hz, 1H), 4.48 (d, J=11.2 Hz, 1H), 4.02 (s, 2H), 3.93-3.76 (m, 6H), 3.65 (dd, J=13.6 Hz & 3.2 Hz, 1H), 3.46 (s, 2H), 3.39 (dd, J=8.8 Hz & 1.6 Hz, 1H), 3.28-3.22 (m, 1H), 2.78 (dd, J=12.4 Hz & 4.0 Hz, 1H), 2.14-2.17 (m, 3H), 1.83-1.69 (m, 3H), 1.52-1.34 (m, 10H).

### Compound 8

### (2R,4S,5R,6R)-2-(benzyloxy)-6-((1R,2R)-3-(2-(4-(10-carboxydecyl)phenyl)acetamido)-1,2-dihydroxypropyl)-4-hydroxy-5-(2-hydroxyacetamido)tetrahydro-2H-pyran-2-carboxylic acid (Cpd. 8)

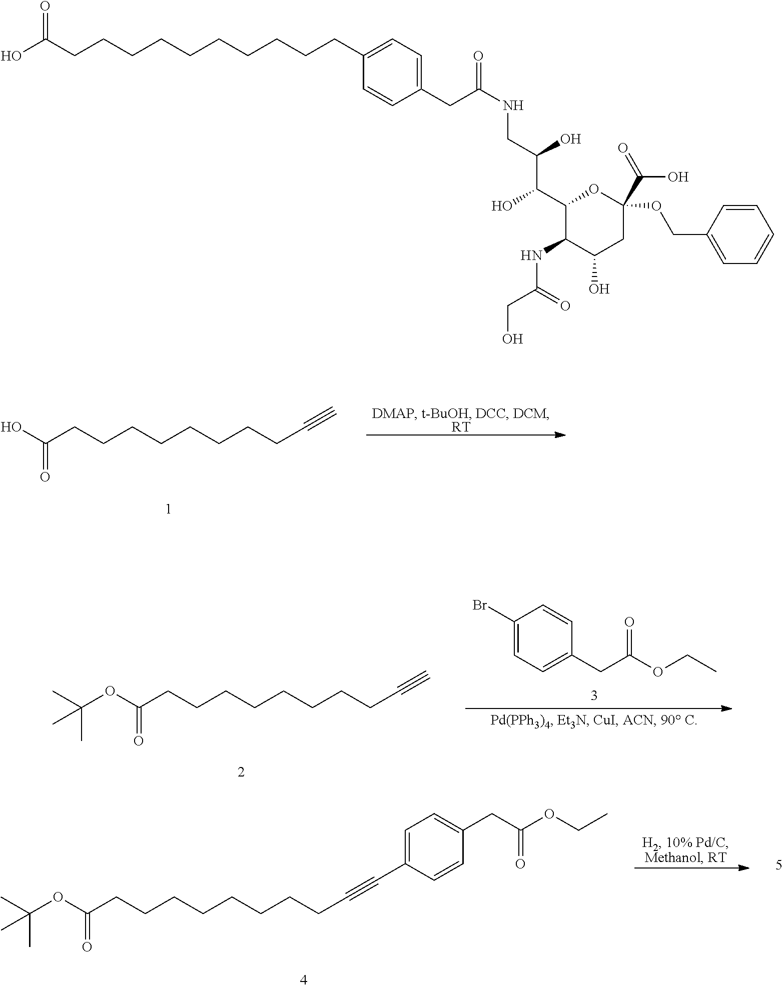

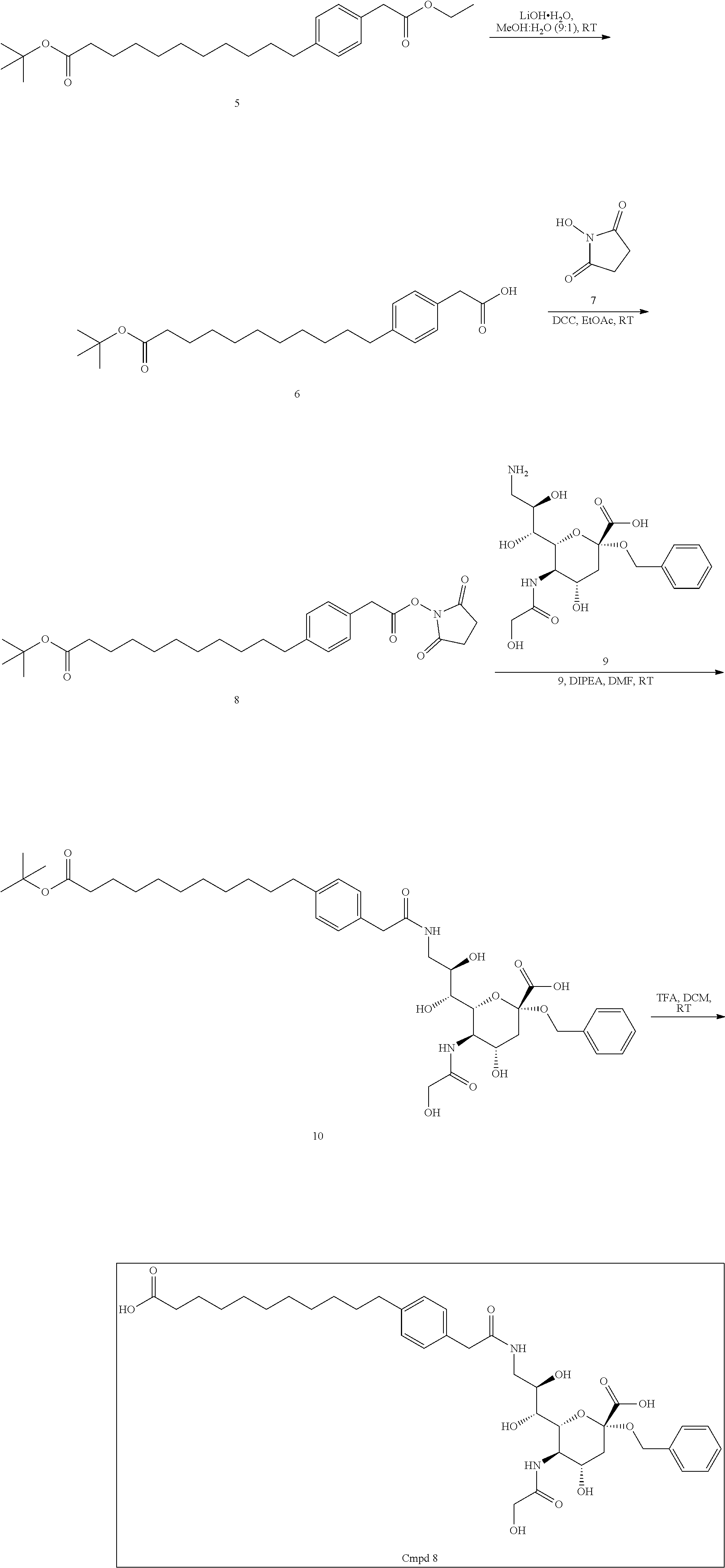

### Synthesis of tert-butyl undec-10-ynoate (2)

To a stirred solution of undec-10-ynoic acid (1, 1.0 g, 5.49 mmol), N,N-dimethylpyridin-4-amine (0.335 g, 2.74 mmol) and 2-methylpropan-2-ol (1.06 mL, 11.0 mmol) in dichloromethane (25 mL) in a 25 ml round bottomed flask equipped with magnetic stirring bar was added N,N′-dicyclohexylmethanediimine (1.70 g, 8.23 mmol) at 0° C. The reaction mixture was stirred for 3 h at room temperature then concentrated under reduced pressure to obtain the desired crude product (2) which was used for the next reaction without further purification. Yield: 1.0 g; 76%.

### Synthesis of tert-butyl 11-(4-(2-ethoxy-2-oxoethyl)phenyl)undec-10-ynoate (4)

A mixture of ethyl 2-(4-bromophenyl)acetate (3, 0.5 g, 2.06 mmol), tetrakis(triphenylphosphane) palladium (119 mg, 0.10 mmol), λ¹-copper(1+) iodide (19.6 mg, 0.10 mmol) and anhydrous acetonitrile (20 mL) was degassed with nitrogen for 10 minutes followed by sequential addition of triethylamine (0.86 mL, 6.17 mmol) and tert-butyl undec-10-ynoate (2, 0.98 g, 4.11 mmol). The reaction mixture was further degassed with nitrogen for 10 minutes, then stirred at 90° C. for 3 h. The reaction mixture was cooled down to room temperature, filtered then concentrated. The crude residue obtained was purified by silica-gel column chromatography using EtOAc and heptane as eluents to afford the desired product (4). Yield: 0.45 g; 53%. LCMS m/z 419.10 [M+H2O]⁺.

### Synthesis of tert-butyl 11-(4-(2-ethoxy-2-oxoethyl)phenyl)undecanoate (5)

To a mixture of tert-butyl 11-(4-(2-ethoxy-2-oxoethyl)phenyl)undec-10-ynoate (4, 0.4 g, 0.99 mmol) in methanol (20 mL, 0.49 mmol) under nitrogen was added 10% Pd—C (0.4 g). The vessel was flushed with H₂(g) then stirred under 100 psi of H₂(g) for 12 h at room temperature. The catalyst was filtered off through celite and the filtrate was concentrated to afford the desired product (5); Yield: 0.3 g; 72%; LCMS m/z 423.25 [M+H2O]⁺. ¹H NMR (400 MHz, CDCl₃): δ 7.19-7.17 (d, J=8.0 Hz, 2H), 7.13-7.11 (d, J=8.0 Hz, 2H), 4.16-4.11 (m, 2H), 3.57 (s, 2H), 2.59-2.55 (t, J=8.0 Hz, 2H), 2.21-2.17 (t, J=8.0 Hz, 2H), 1.58-1.54 (m, 4H), 1.44 (s, 9H), 1.26-1.23 (m, 15H).

### Synthesis of 2-(4-(11-(tert-butoxy)-11-oxoundecyl)phenyl)acetic acid (6)

To a stirred solution of tert-butyl 11-(4-(2-ethoxy-2-oxoethyl)phenyl)undecanoate (5, 0.3 g, 0.74 mmol) in methanol (6 mL) was added a solution of lithium hydroxide (26.6 mg, 1.11 mmol) in water (2 mL) drop-wise at 0° C. The mixture was stirred for 3 h at room temperature then concentrated under reduced pressure. The residue obtained was neutralized with citric acid, extracted with ethyl acetate then concentrated. The solid residue obtained was triturated with pentane then ether to afford the desired product (6). Yield: 200 mg; 71%; LCMS m/z 376.10 [M+H]⁺.

### Synthesis of tert-butyl 11-(4-(2-((2,5-dioxopyrrolidin-1-yl)oxy)-2-oxoethyl)phenyl)undecanoate (8)

To a stirred solution of 2-(4-(11-(tert-butoxy)-11-oxoundecyl)phenyl)acetic acid (6, 0.2 g, 494 μmol) in ethyl acetate (5.05 mL, 51.3 mmol) was added N,N′-dicyclohexylmethanediimine (0.20 g, 0.99 mmol) at 0° C. After 10 minutes of stirring at 0° C. 1-hydroxy-2,5-pyrrolidinedione (7, 0.12 g, 0.99 mmol) was added and the reaction mixture was stirred at room temperature for 6 h. The reaction mixture was concentrated and the crude residue obtained was purified by silica-gel column chromatography using ethyl acetate and heptane as eluents to afford the desired product (8). Yield: 110 g; 40%; LCMS m/z 492.20 [M+H2O]⁺. ¹H NMR (400 MHz, CDCl₃): δ 7.23 (d, J=8.0 Hz, 2H), 7.16 (d, J=8.0 Hz, 2H), 3.90 (s, 2H), 2.83 (s, 4H), 2.58 (t, J=8.0 Hz, 2H), 2.19 (t, J=8.0 Hz, 2H), 1.58-1.56 (m, 7H), 1.44 (s, 9H), 1.26 (m, 12H).

### Synthesis of (2R,4S,5R,6R)-6-[(1R,2R)-3-({[p-(10-tert-butoxycarbonyldecyl)phenyl]methyl}carbonylamino)-1,2-dihydroxypropyl]-2-(benzyloxy)-4-hydroxy-5-[(hydroxymethyl)carbonylamino]tetrahydro-2H-pyran-2-carboxylic acid (10)

To a mixture of 6-(3-amino-1,2-dihydroxypropyl)-2-(benzyloxy)-4-hydroxy-5-[(hydroxymethyl)carbonylamino]tetrahydro-2H-pyran-2-carboxylic acid (9, 0.22 g, 0.48 mmol) and [p-(10-tert-butoxycarbonyl-1-decynyl)phenyl]acetic acid (8, 0.17 g, 0.424 mmol) in dimethylformamide (4.8 mL) at 0° C. was added DIEA (0.337 mL, 1.93 mmol) dropwise. The reaction mixture to stir at room temperature for 4 h then concentrated to afford the desired crude product (10). Yield: 0.2 g; 54%; LCMS m/z 773.15 [M+H]⁺.

### Synthesis of (2R,4S,5R,6R)-2-(benzyloxy)-6-((1R,2R)-3-(2-(4-(10-carboxydecyl)phenyl)acetamido)-1,2-dihydroxypropyl)-4-hydroxy-5-(2-hydroxyacetamido)tetrahydro-2H-pyran-2-carboxylic acid (Cpd. 8)

To a stirred solution of (2R,4S,5R,6R)-6-[(1R,2R)-3-({[p-(10-tert-butoxycarbonyldecyl)phenyl]methyl}carbonylamino)-1,2-dihydroxypropyl]-2-(benzyloxy)-4-hydroxy-5-[(hydroxymethyl)carbonylamino]tetrahydro-2H-pyran-2-carboxylic acid (10, 0.2 g, 0.11 mmol) and anhydrous dichloromethane (3.0 mL) at 0° C. was added trifluoroacetic acid (0.5 mL) drop-wise. The mixture was stirred for 2 h at rt then concentrated. The crude residue obtained was purified by prep-HPLC to afford the desired product (Cpd. 8). Yield: 0.026 g; 33%; LCMS m/z 717.60 [M+H]⁺; ¹H-NMR (400 MHz, methanol-d₄): δ 7.93 (d, J=7.6 Hz, 1H), 7.32-7.25 (m, 5H), 7.18 (d, J=8 Hz, 2H), 7.09-7.07 (d, J=8.0 Hz, 2H), 4.79 (d, J=11.2 Hz, 1H), 4.48 (d, J=11.2 Hz, 1H), 4.02 (s, 2H), 3.93-3.79 (m, 4H), 3.67 (dd, J=13.6 Hz & 3.2 Hz, 1H), 3.49 (s, 2H), 3.40 (d, J=9.2 Hz, 1H), 3.26-3.21 (m, 1H), 2.78 (dd, J=8.0 Hz & 4.4 Hz, 1H), 2.53 (t, J=7.6 Hz, 2H), 2.27 (t, J=7.2 Hz, 1H), 1.80 (t, J=12.0 Hz, 1H), 1.60-1.55 (m, 4H), 1.29 (m, 12H).

### Compound 9

### (2R,4S,5R,6R)-2-(benzyloxy)-6-((1R,2R)-1,2-dihydroxy-3-(2-(4-((2-(2-(prop-2-yn-1-yloxy)ethoxy)ethoxy)methyl)phenyl)acetamido)propyl)-4-hydroxy-5-(2-hydroxyacetamido)tetrahydro-2H-pyran-2-carboxylic acid (Cpd. 9)

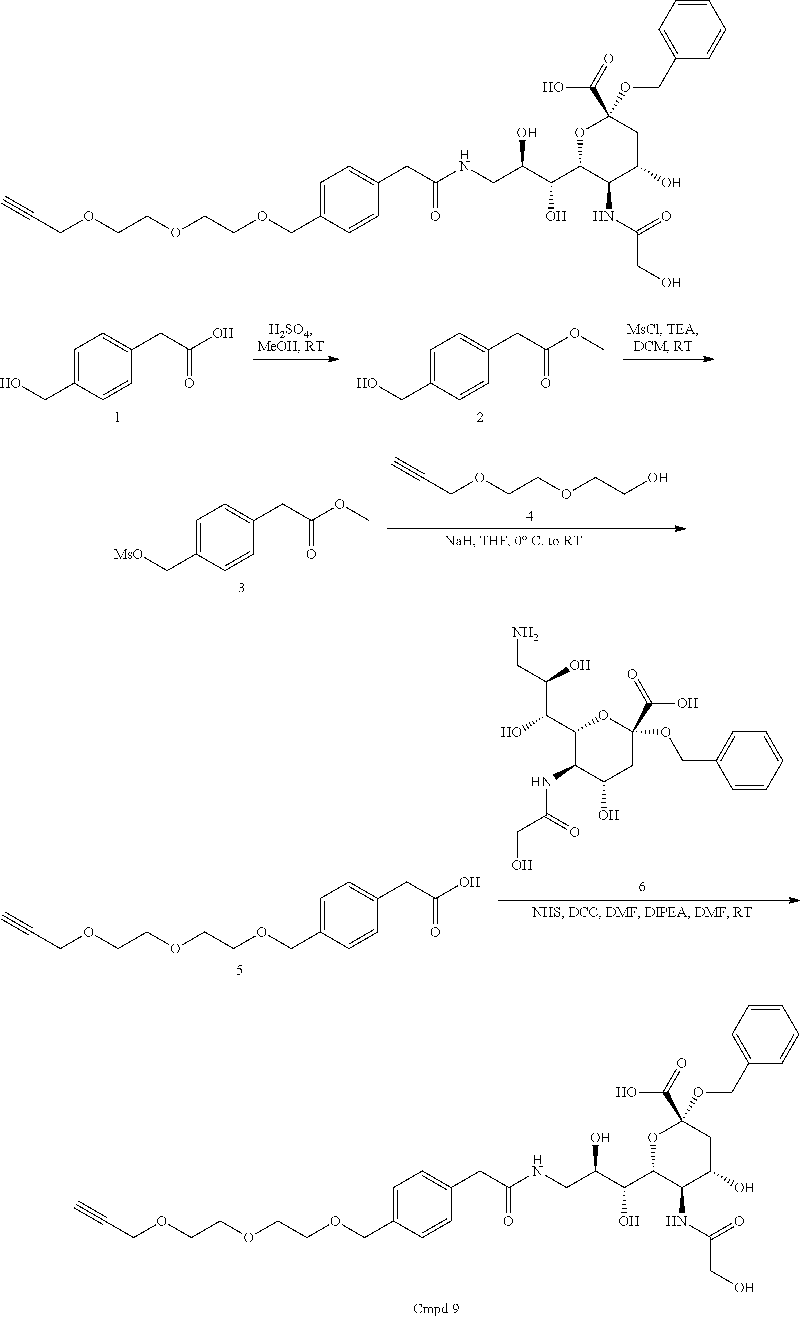

### Synthesis of methyl 2-(4-(hydroxymethyl)phenyl)acetate (2)

To a stirred solution of 2-(4-(hydroxymethyl)phenyl)acetic acid (1, 1.0 g, 6.02 mmol) in methanol (10.0 mL) was added sulfuric acid (2.0 mL) at 0° C. and the reaction mixture was heated at 60° C. for 3 h. The reaction mixture was cooled down to room temperature then concentrated. The crude residue obtained was diluted with an aqueous saturated solution of sodium bicarbonate and extracted with ethyl acetate. The combined organic layers were dried, filtered and concentrated to afford the desired product (2). Yield: 0.98 g, 90%; LCMS m/z 198.20 [M+H2O]+.

### Synthesis of methyl 2-(4-(((methylsulfonyl)oxy)methyl)phenyl)acetate (3)

To a stirred solution of methyl 2-(4-(hydroxymethyl)phenyl)acetate (2, 0.950 g, 4.99 mmol) and trimethyl amine (1.39 mL, 9.98 mmol) in dichloromethane (10.0 mL) was added methane sulfonyl chloride (0.46 mL, 5.98 mmol) was added at 0° C. and the reaction mixture was stirred at room temperature for 2 h. The reaction mixture was quenched with water and extracted with ethyl acetate. The organic layer was dried, filtered and concentrated to afford the desired crude product. Yield: 0.900 g (crude).

### Synthesis of 2-(4-((2-(2-(prop-2-yn-1-yloxy)ethoxy)ethoxy)methyl)phenyl)acetic acid (5)

To a stirred solution of 2-(2-(prop-2-yn-1-yloxy)ethoxy)ethan-1-ol (4, 0.50 g, 3.48 mmol) in N, N-dimethylformamide (10.0 mL) was added sodium hydride (60%) (0.160 g, 4.17 mmol) at 0° C. and stirred at the same temperature for 30 min. Methyl 2-(4-(((methylsulfonyl)oxy)methyl)phenyl)acetate (3, 0.900 g, 0.3.48 mmol) was added at 0° C. and the mixture was stirred at room temperature for another 12 h. The reaction mixture was quenched with water and extracted by ethyl acetate. The organic layer was dried, filtered and concentrated. The crude residue obtained was purified by prep HPLC using water and acetonitrile as eluents (+0.1% TFA) to afford the desired product (5). Yield: 0.030 g, 3%; LCMS m/z 293.18 [M+H]+.

### Synthesis of (2R,4S,5R,6R)-2-(benzyloxy)-6-((1R,2R)-1,2-dihydroxy-3-(2-(4-((2-(2-(prop-2-yn-1-yloxy)ethoxy)ethoxy)methyl)phenyl)acetamido)propyl)-4-hydroxy-5-(2-hydroxyacetamido)tetrahydro-2H-pyran-2-carboxylic acid (Cpd. 9)

To a stirred solution of 2-(4-((2-(2-(prop-2-yn-1-yloxy)ethoxy)ethoxy)methyl)phenyl)acetic acid (5, 0.030 g, 0.102 mmol) in dimethylformamide (2.0 mL), were successively added N,N′-Dicyclohexylcarbodiimide (0.021 g, 0.102 mmol) and N-hydroxysuccinimide (0.011 g, 0.102 mmol) at 0° C. After 3 h of stirring at room temperature, (2R,4S,5R,6R)-6-((1R,2R)-3-amino-1,2-dihydroxypropyl)-2-(benzyloxy)-4-hydroxy-5-(2-hydroxyacetamido)tetrahydro-2H-pyran-2-carboxylic acid (6, 0.042 g, 0.102 mmol) and ethylbis(propan-2-yl)amine (0.10 mL, 0.51 mmol) were successively added at 0° C. and the reaction mixture was stirred at room temperature for another 4 h. The reaction mixture was concentrated and the crude residue obtained was purified by prep HPLC using water and acetonitrile (+0.1% TFA) as eluents to afford the desired product (Cpd. 9). Yield: 0.004 g, 6%; LCMS m/z 689.40 [M+H]+. 1H NMR (400 MHz, methanol-d4): δ 7.35-7.25 (m, 9H), 4.80 (d, J=11.2 Hz, 1H), 4.51-4.49 (m, 3H), 4.17 (d, J=2.4 Hz, 2H), 4.01 (s, 2H), 3.92-3.74 (m, 4H), 3.67-3.37 (m, 9H), 3.36 (s, 2H), 3.32 (dd, J=6.0 Hz & 1.6 Hz, 1H), 3.27-3.22 (m, 1H), 2.84-2.80 (m, 2H), 1.79-1.73 (m, 1H).

### Compound 10

### (2R,4S,5R,6R)-2-((6-aminohexyl)oxy)-6-((1R,2R)-3-(2-(4-ethynylphenyl)acetamido)-1,2-dihydroxypropyl)-4-hydroxy-5-(2-hydroxyacetamido)tetrahydro-2H-pyran-2-carboxylic acid (Cpd. 10)

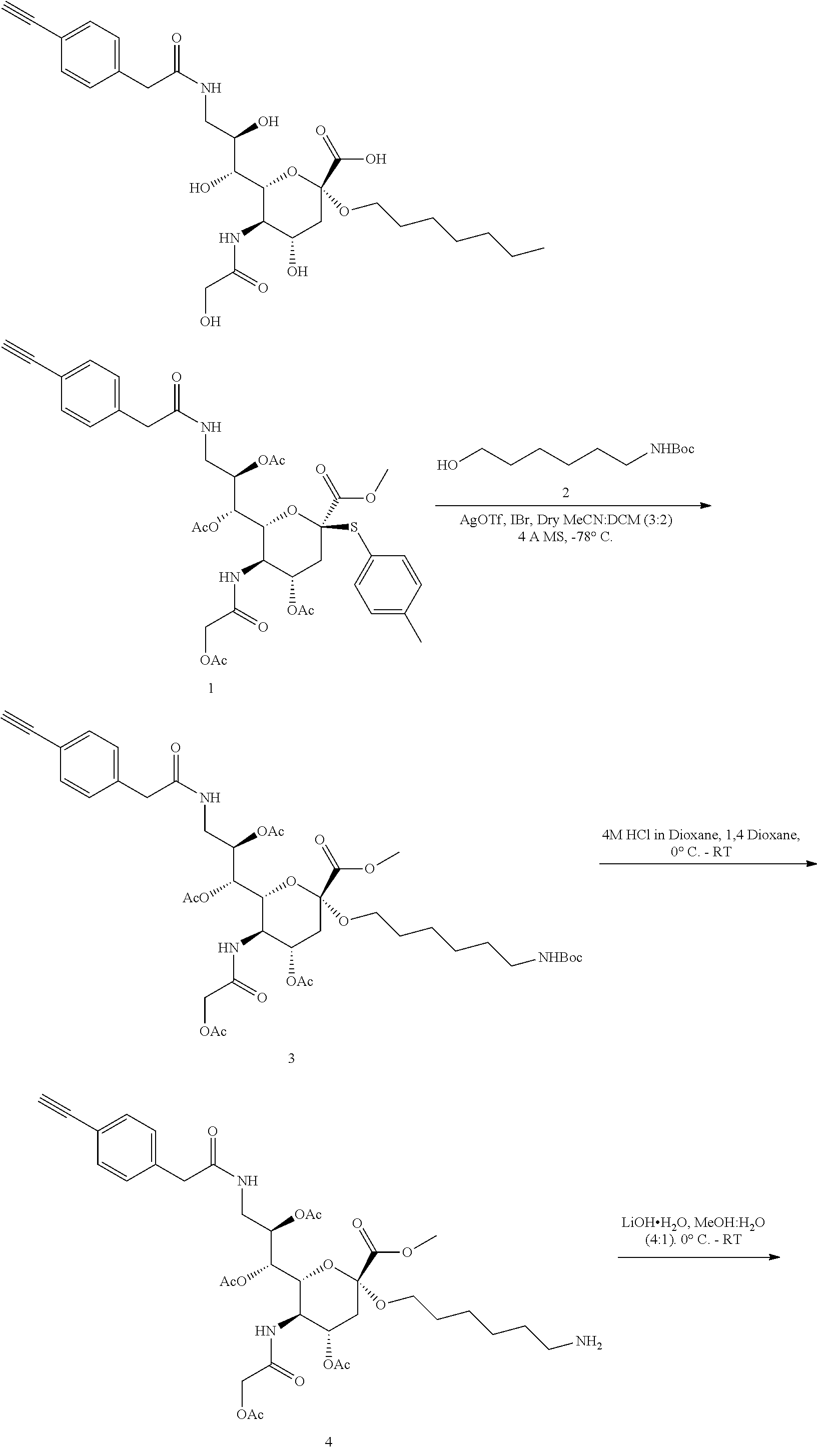

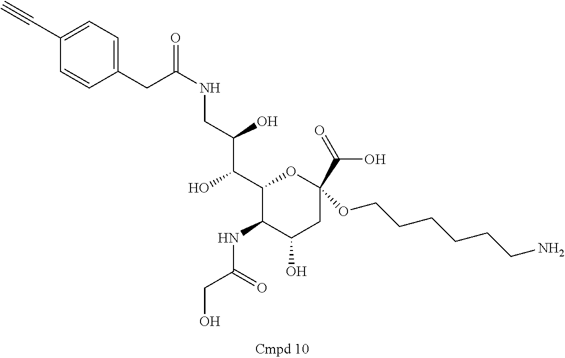 [SMILES: C#Cc1ccc(CC(=O)NC[C@@H](O)[C@@H](O)[C@@H]2O[C@@](OCCCCCCN)(C(=O)O)C[C@H](O)[C@H]2NC(=O)CO)cc1]

### Synthesis of (1R,2R)-1-((2R,3R,4S,6R)-4-acetoxy-3-(2-acetoxyacetamido)-6-((6-((tert-butoxycarbonyl)amino)hexyl)oxy)-6-(methoxycarbonyl)tetrahydro-2H-pyran-2-yl)-3-(2-(4-ethynylphenyl)acetamido)propane-1,2-diyl diacetate (3)

A mixture of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-ethynylphenyl)acetamido)propane-1,2-diyl diacetate (1, 0.60 g, 0.79 mmol), tert-butyl (6-hydroxyhexyl)carbamate (2, 0.86 g, 3.97 mmol), silver(1+) trifluoromethanesulfonate (0.61 g, 2.38 mmol) and activated 4 Å powdered molecular sieves (1.00 g) in anhydrous dichloromethane (55.0 mL) and anhydrous acetonitrile (90.0 mL) was stirred at room temperature for 1 h. The mixture was cooled to −78° C. and a solution of iodobromane (0.33 g, 1.59 mmol) in dichloromethane (5.0 mL) was added dropwise. After 2 h −78° C., triethylamine (1.0 mL) was added drop-wise and the cooling bath was removed to allow gradual warming up to room temperature. The reaction mixture was filtered, the filtrate was washed with and aqueous saturated solution of sodium bicarbonate, dried, filtered and concentrated. The crude residue obtained was purified by silicas-gel column chromatography using ethyl acetate and heptane as eluents to afford the desired product (3). Yield: 0.46 g, 66.17%; LCMS m/z 848.05 [M+H]+.

### Synthesis of (1R,2R)-1-((2R,3R,4S,6R)-4-acetoxy-3-(2-acetoxyacetamido)-6-((6-aminohexyl)oxy)-6-(methoxycarbonyl)tetrahydro-2H-pyran-2-yl)-3-(2-(4-ethynylphenyl)acetamido)propane-1,2-diyl diacetate (4)

To a stirred solution of (1R,2R)-1-((2R,3R,4S,6R)-4-acetoxy-3-(2-acetoxyacetamido)-6-((6-((tert-butoxycarbonyl)amino)hexyl)oxy)-6-(methoxycarbonyl)tetrahydro-2H-pyran-2-yl)-3-(2-(4-ethynylphenyl)acetamido)propane-1,2-diyl diacetate (3, 0.35 g, 0.32 mmol) in anhydrous 1,4-dioxane (5.0 mL) was added 4 M HCl in dioxane (2.0 mL) drop-wise at 0° C. The reaction mixture was stirred at room temperature for 3 h then concentrated to afford the desired crude product (4). Yield: 0.30 g, Crude; LCMS (ESI) m/z 747.95 [M+H]+.

### Synthesis of (2R,4S,5R,6R)-6-((1R,2R)-3-(2-(4-ethynylphenyl)acetamido)-1,2-dihydroxypropyl)-4-hydroxy-5-(2-hydroxyacetamido)-2-((6-methoxyhexyl)oxy)tetrahydro-2H-pyran-2-carboxylic acid (Cpd. 10)

To a stirred solution of (1R,2R)-1-((2R,3R,4S,6R)-4-acetoxy-3-(2-acetoxyacetamido)-6-((6-aminohexyl)oxy)-6-(methoxycarbonyl)tetrahydro-2H-pyran-2-yl)-3-(2-(4-ethynylphenyl)acetamido)propane-1,2-diyl diacetate (4, 0.15 g, 0.20 mmol) in methanol (5.0 mL) and water (0.5 mL) was added lithium hydroxide monohydrate (0.027 g, 1.20 mmol) at 0° C. The reaction mixture was stirred at room temperature for 6 h then treated with acidic (Dowex 50, H+) to pH˜6. The suspension was filtered and the filtrate was concentrated. The crude residue obtained was purified by prep HPLC using Acetonitrile and water (+0.1% TFA) as eluents to afford the desired product (Cpd. 10). Yield: 0.018 g, 13.22%; LCMS m/z 566.45 [M+H]+. 1H NMR (400 MHz, MeOD-d4): δ 7.40 (d, J=8.0 Hz, 2H), 7.29 (d, J=8.0 Hz, 2H), 4.03 (s, 2H), 3.86-3.81 (m, 2H), 3.79-3.69 (m, 2H), 3.65 (dd, J=10.4 Hz & 1.6 Hz, 1H), 3.59-3.53 (m, 2H), 3.49-3.40 (m, 2H), 3.36-3.34 (m, 2H), 3.06-3.01 (m, 1H), 2.92-2.80 (m, 2H), 2.74 (dd, J=12.4 Hz & 3.6 Hz, 1H), 1.65 (t, J=12.0 Hz, 1H), 1.59-1.51 (m, 4H), 1.43-1.29 (m, 4H).

### Compound 11

### (2R,4S,5R,6R)-6-((1R,2R)-3-(2-(4-ethynylphenyl)acetamido)-1,2-dihydroxypropyl)-4-hydroxy-5-(2-hydroxyacetamido)-2-((6-methoxyhexyl)oxy)tetrahydro-2H-pyran-2-carboxylic acid (Cpd. 11)

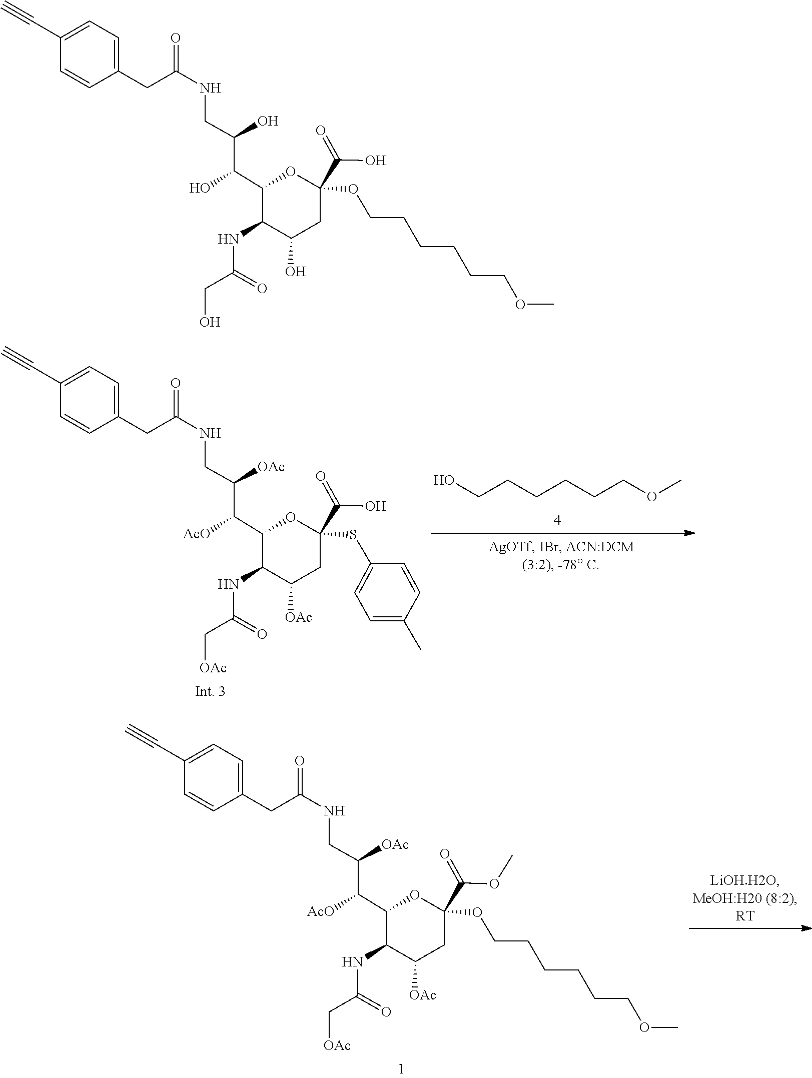

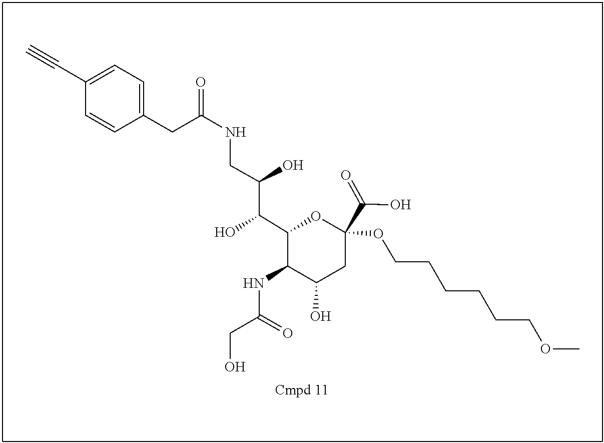 [SMILES: C#Cc1ccc(CC(=O)NC[C@@H](O)[C@@H](O)[C@@H]2O[C@@](OCCCCCCOC)(C(=O)O)C[C@H](O)[C@H]2NC(=O)CO)cc1]

### Synthesis of (1R,2R)-1-((2R,3R,4S,6R)-4-acetoxy-3-(2-acetoxyacetamido)-6-(methoxycarbonyl)-6-((6-methoxyhexyl)oxy)tetrahydro-2H-pyran-2-yl)-3-(2-(4-ethynylphenyl)acetamido)propane-1,2-diyl diacetate (1)

A mixture of (1R,2R)-1-((2R,3R,4S,6S)-4-acetoxy-3-(2-acetoxyacetamido)-6-(methoxycarbonyl)-6-(p-tolylthio)tetrahydro-2H-pyran-2-yl)-3-(2-(4-ethynylphenyl)acetamido)propane-1,2-diyl diacetate (Int. 3, 0.450 g, 0.596 mmol), 6-methoxyhexan-1-ol (4, 0.394 g, 2.98 mmol), silver(1+) trifluoromethanesulfonate (0.306 g, 1.19 mmol) and activated 4 Å powdered molecular sieves (1.00 g) in anhydrous dichloromethane (9.0 mL) and anhydrous acetonitrile (15.0 mL) was stirred at room temperature for 1 h. The mixture was cooled to −78° C. and a solution of iodobromane (0.185 g, 0.894 mmol) in dichloromethane (1.0 mL) was added dropwise. After 2 h of stirring at −78° C. the reaction mixture was quenched by addition of triethylamine (1.0 mL) then the cooling bath was removed to allow the mixture to warm up to room temperature. The reaction mixture was filtered, the filtrate was washed with a saturated solution of sodium bicarbonate, dried over sodium sulfate, filtered and concentrated. The crude residue obtained was purified by silica-gel column chromatography ethyl acetate and heptane as eluents to afford the desired product (1). Yield: 0.35 g, 77%; LCMS m/z 763.15 [M+H]+.

### Synthesis of (2R,4S,5R,6R)-6-((1R,2R)-3-(2-(4-ethynylphenyl)acetamido)-1,2-dihydroxypropyl)-4-hydroxy-5-(2-hydroxyacetamido)-2-((6-methoxyhexyl)oxy)tetrahydro-2H-pyran-2-carboxylic acid (Cpd. 11)

To a stirred solution of (1R,2R)-1-((2R,3R,4S,6R)-4-acetoxy-3-(2-acetoxyacetamido)-6-(methoxycarbonyl)-6-((6-methoxyhexyl)oxy)tetrahydro-2H-pyran-2-yl)-3-(2-(4-ethynylphenyl)acetamido)propane-1,2-diyl diacetate (1, 0.30 g, 0.393 mmol) in methanol (4.0 mL) and water (0.5 mL) was added a solution of lithium hydroxide monohydrate (0.101 g, 2.35 mmol) at 0° C. The reaction mixture was stirred at room temperature for 6 h then treated with acidic (Dowex 50, H+) to pH˜6. The suspension was filtered, the filtrate was concentrated, and the crude residue obtained was purified by prep HPLC using acetonitrile and water (+0.1% TFA) as eluents to afford the desired product (Cpd. 11). Yield: 0.030 g, 13%; LCMS m/z 581.40 [M+H]+. 1H NMR (400 MHz, MeOD-d4): δ 7.39 (d, J=8.4 Hz, 2H), 7.28 (d, J=8.0 Hz, 2H), 4.01 (s, 2H), 3.89-3.86 (m, 1H), 3.85-3.78 (m, 2H), 3.77-3.68 (m, 2H), 3.67-3.63 (m, 1H), 3.54 (s, 2H), 3.44 (s, 1H), 3.39-3.35 (m, 4H), 3.31-3.30 (m, 2H), 3.25-3.20 (m, 1H), 2.71 (dd, J=12.4 Hz & 3.6 Hz, 1H), 1.71 (t, J=12.0 Hz, 1H), 1.55-1.54 (m, 4H), 1.36-1.34 (m, 4H).

### Compound 12

### (2S,4S,5R,6R)-2-(benzylthio)-6-((1R,2R)-3-(2-(4-ethynylphenyl)acetamido)-1,2-dihydroxypropyl)-4-hydroxy-5-(2-hydroxyacetamido)tetrahydro-2H-pyran-2-carboxylic acid (Cpd. 12)

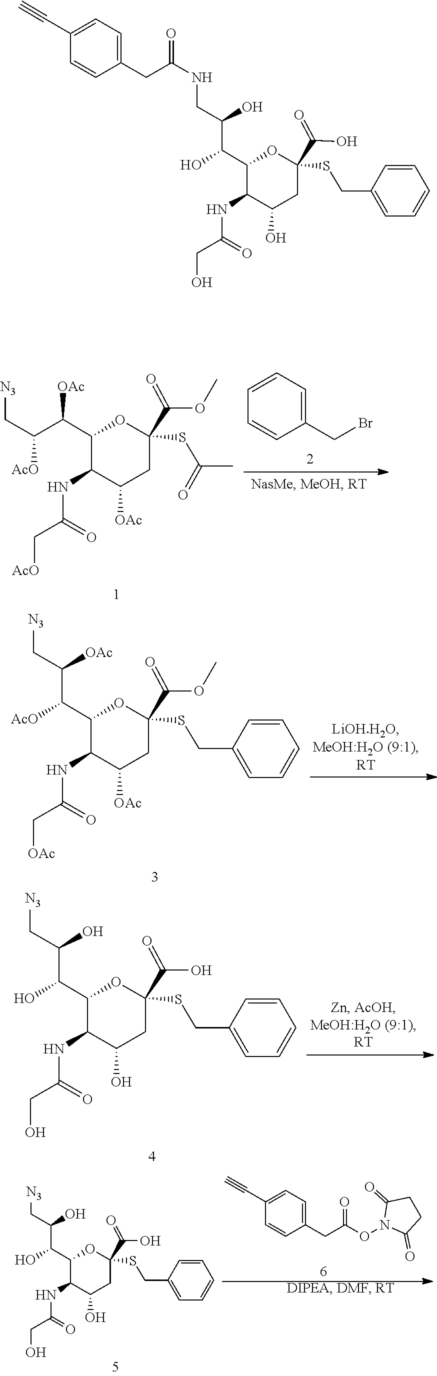

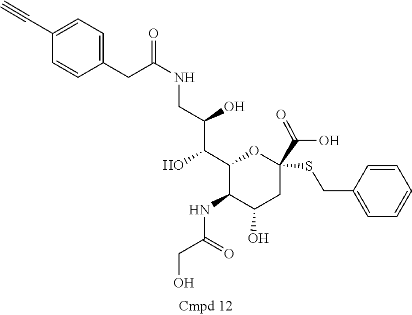 [SMILES: C#Cc1ccc(CC(=O)NC[C@@H](O)[C@@H](O)[C@@H]2O[C@@](SCc3ccccc3)(C(=O)O)C[C@H](O)[C@H]2NC(=O)CO)cc1]

### Synthesis of (1R,2R)-1-((2R,3R,4S,6S)-4-acetoxy-3-(2-acetoxyacetamido)-6-(benzylthio)-6-(methoxycarbonyl)tetrahydro-2H-pyran-2-yl)-3-azidopropane-1,2-diyl diacetate (3)

To a stirred solution of (1R,2R)-1-((2R,3R,4S,6S)-4-acetoxy-3-(2-acetoxyacetamido)-6-(acetylthio)-6-(methoxycarbonyl)tetrahydro-2H-pyran-2-yl)-3-azidopropane-1,2-diyl diacetate (1, 0.230 g, 0.389 mmol) in methanol (5.0 mL) was added sodium thiomethoxide (0.039 g, 0.466 mmol) at 0° C. After 1 h of stirring, (bromomethyl)benzene (2, 0.133 g, 0.778 mmol) was added at 0° C. and the reaction mixture was stirred at room temperature for another 1 h. After completion the reaction mixture was concentrated. The crude residue obtained was taken up in ethyl acetate and the resulting solution was washed with water. The organic layer was separated, dried, filtered and concentrated. The crude residue obtained was purified by silica-gel column chromatography using ethyl acetate and heptane as eluents to afford the desired product (3). Yield: 0.20 g, 80%; LCMS (ESI) m/z 638.95 [M+H]+.

### Synthesis of (2S,4S,5R,6R)-6-((1R,2R)-3-azido-1,2-dihydroxypropyl)-2-(benzylthio)-4-hydroxy-5-(2-hydroxyacetamido)tetrahydro-2H-pyran-2-carboxylic acid (4)

To a stirred solution of (1R,2R)-1-((2R,3R,4S,6S)-4-acetoxy-3-(2-acetoxyacetamido)-6-(benzylthio)-6-(methoxycarbonyl)tetrahydro-2H-pyran-2-yl)-3-azidopropane-1,2-diyl diacetate (3, 0.20 g, 0.313 mmol) in methanol (4.0 mL) and water (1.0 mL) was added lithium hydroxide monohydrate (0.067 g, 01.56 mmol) at 0° C. drop-wise. The reaction mixture was stirred at room temperature for 6 h then treated with acidic (Dowex 50, H+) to pH˜6. The suspension was filtered and the filtrate was concentrated. The crude residue obtained was purified by trituration with diethyl ether to afford the desired product (5). Yield: 0.140 g, 98%; LCMS m/z 457.00 [M+H]+.

### Synthesis of (2S,4S,5R,6R)-6-((1R,2R)-3-amino-1,2-dihydroxypropyl)-2-(benzylthio)-4-hydroxy-5-(2-hydroxyacetamido)tetrahydro-2H-pyran-2-carboxylic acid (5)

To a stirred solution of (2S,4S,5R,6R)-6-((1R,2R)-3-azido-1,2-dihydroxypropyl)-2-(benzylthio)-4-hydroxy-5-(2-hydroxyacetamido)tetrahydro-2H-pyran-2-carboxylic acid (4, 0.170 g, 0.372 mmol) in methanol (3.0 mL) and water (0.50 mL), were successively added zinc (0.243 g, 3.72 mmol) and acetic acid (0.148 mL, 3.72 mmol) very slowly at 0° C. The reaction mixture was stirred at room temperature for 2 h then filtered. The filtrate was concentrated, and the crude residue obtained was triturated with diethyl ether to afford the desired product (5). Yield: 0.150 g, 93%; LCMS (ESI) m/z 431.00 [M+H]+.

### Synthesis of (2S,4S,5R,6R)-2-(benzylthio)-6-((1R,2R)-3-(2-(4-ethynylphenyl)acetamido)-1,2-dihydroxypropyl)-4-hydroxy-5-(2-hydroxyacetamido)tetrahydro-2H-pyran-2-carboxylic acid (Cpd. 12)

To a stirred solution of (2S,4S,5R,6R)-6-((1R,2R)-3-amino-1,2-dihydroxypropyl)-2-(benzylthio)-4-hydroxy-5-(2-hydroxyacetamido)tetrahydro-2H-pyran-2-carboxylic acid (5, 0.170 g, 0.394 mmol) and 2,5-dioxopyrrolidin-1-yl 2-(4-ethynylphenyl)acetate (6, 0.101 g, 0.394 mmol) in dimethylformamide (3.0 mL) at 0° C. was added DIEA (0.34 mL, 1.97 mmol) and the reaction mixture was stirred at room temperature for 4 h. The mixture was concentrated and the crude residue obtained was purified by prep HPLC using water and acetonitrile (+0.1% TFA) as eluents to afford the desired product (Cpd. 12). Yield: 0.050 g, 22%; LCMS m/z 573.30 [M+H]+. 1H NMR (400 MHz, methanol-d4): δ 7.35-7.20 (m, 9H), 4.03-4.00 (m, 3H), 3.89-3.81 (m, 4H), 3.66-3.62 (m, 2H), 3.53 (s, 2H), 3.43 (s, 1H), 3.37 (dd, J=8.8 Hz & 1.2 Hz, 1H), 3.24-3.19 (m, 1H), 2.78 (dd, J=12.8 Hz & 4.0 Hz, 1H), 1.79 (t, J=12.8 Hz, 1H).

### Compound 13

### (2R,4S,5R,6R)-2-(benzyloxy)-6-((1R,2R)-1,2-dihydroxy-3-(2-(4-(7-(prop-2-yn-1-yloxy)heptyl)phenyl)acetamido)propyl)-4-hydroxy-5-(2-hydroxyacetamido)tetrahydro-2H-pyran-2-carboxylic acid (Cpd. 13)

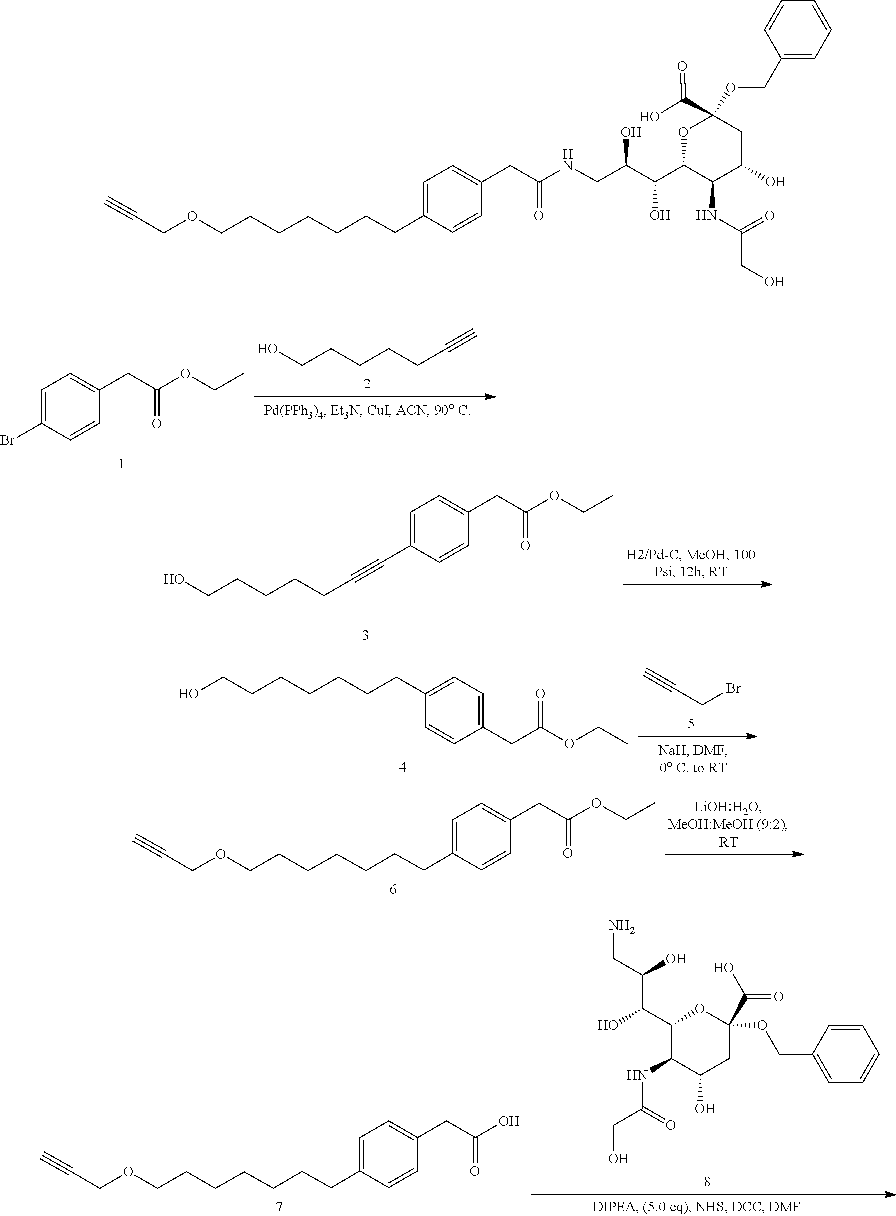

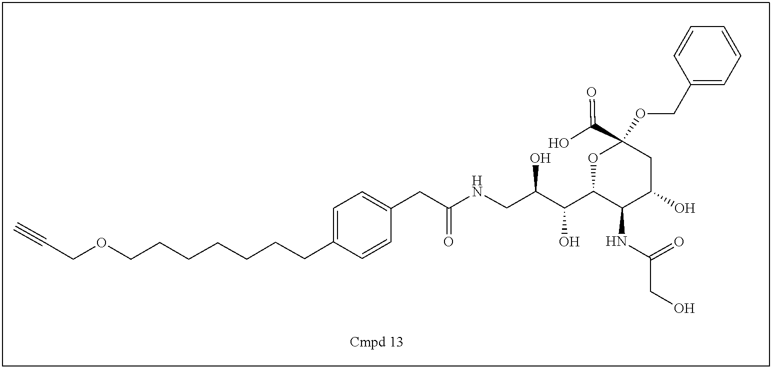 [SMILES: C#CCOCCCCCCCc1ccc(CC(=O)NC[C@@H](O)[C@@H](O)[C@@H]2O[C@@](OCc3ccccc3)(C(=O)O)C[C@H](O)[C@H]2NC(=O)CO)cc1]

### Synthesis of ethyl 2-(4-(7-hydroxyhept-1-yn-1-yl)phenyl)acetate (3)

To a stirred solution of ethyl 2-(4-bromophenyl)acetate (1, 1.0 g, 4.11 mmol) in acetonitrile (10.0 mL) were successively added 6-heptyn-1-ol (2, 1.38 g, 12.30 mmol), triethylamine (1.73 mL, 12.30 mmol), tetrakis(triphenylphosphane) palladium (0.475 g, 0.411 mmol) and copper(1+) iodide (0.078 g, 0.411 mmol) at room temperature. The mixture was degassed by sparging with nitrogen gas for 10 minutes, then stirred at 90° C. for 3 h. The reaction mixture was cooled to room temperature, filtered, and the filtrate was concentrated. The crude residue obtained was purified by silica-gel column chromatography using ethyl acetate and heptane as eluents to afford the desired product (3). Yield: 0.580 g, 51%; LCMS m/z 292.15 [M+H]+.

### Synthesis of ethyl 2-(4-(7-hydroxyheptyl)phenyl)acetate (4)

To a stirred solution of ethyl 2-(4-(7-hydroxyhept-1-yn-1-yl)phenyl)acetate (2, 0.50 g, 1.82 mmol) in methanol (10.0 mL) under nitrogen was added 10% Pd—C (0.50 g). The reaction mixture was stirred under 100 psi of hydrogen gas for 12 h then the catalyst was filtered off through celite and the filtrate was concentrated. The crude residue obtained was purified by silica-gel column chromatography using ethyl acetate and heptane as eluents to afford the desired product (4). Yield: 0.350 g, 69%; LCMS m/z 279.15 [M+H]+.

### Synthesis of ethyl 2-(4-(7-(prop-2-yn-1-yloxy)heptyl)phenyl)acetate (6)

To a stirred solution of ethyl 2-(4-(7-hydroxyheptyl)phenyl)acetate (4, 0.350 g, 1.26 mmol) in N, N-dimethylformamide (5.0 mL), was added sodium hydride (60%) (0.034 g, 1.51 mmol) at 0° C. After 30 min of stirring, 3-bromoprop-1-yne (0.054 mL, 0.718 mmol) was added and the reaction mixture was stirred at 0° C. for another 12 h. The reaction mixture was carefully quenched by addition of water and extracted with ethyl acetate. The combined organic layers were dried, filtered, and concentrated. The crude residue obtained was purified by silica-gel column chromatography using ethyl acetate and heptane as eluents to afford the desired product (6). Yield: 0.260 g, 65%; LCMS m/z 317.10 [M+H]+.

### Synthesis of 2-(4-(7-(prop-2-yn-1-yloxy)heptyl)phenyl)acetic acid (7)

To a stirred solution of ethyl 2-(4-(7-(prop-2-yn-1-yloxy)heptyl)phenyl)acetate (6, 0.260 g, 0.8221 mmol) in ethanol (4.0 mL) at 0° C. was added a solution of lithium hydroxide monohydrate (0.022 g, 0.520 mmol) in water (1.0 ml) drop-wise and the reaction mixture was stirred at room temperature for 6 h. After completion the reaction mixture was treated with 1N HCl solution and extracted with ethyl acetate. The organic layer was concentrated to afford the crude desired product (7). Yield: 0.200 g, 84%; LCMS m/z 289.10 [M+H]+.

### Synthesis of (2R,4S,5R,6R)-2-(benzyloxy)-6-((1R,2R)-1,2-dihydroxy-3-(2-(4-(7-(prop-2-yn-1-yloxy)heptyl)phenyl)acetamido)propyl)-4-hydroxy-5-(2-hydroxyacetamido)tetrahydro-2H-pyran-2-carboxylic acid (Cpd. 13)

To a stirred solution of 2-(4-(7-(prop-2-yn-1-yloxy)heptyl)phenyl)acetic acid (7, 0.050 g, 0.173 mmol) in dimethylformamide (2.0 mL) at 0° C. were successively added N,N′-dicyclohexylcarbodiimide (0.037 g, 0.173 mmol) and N-Hydroxysuccinimide (0.019 g, 0.173 mmol). After 3 h of stirring, (2R,4S,5R,6R)-6-((1R,2R)-3-amino-1,2-dihydroxypropyl)-2-(benzyloxy)-4-hydroxy-5-(2-hydroxyacetamido)tetrahydro-2H-pyran-2-carboxylic acid (8, 0.071 g, 0.173 mmol) and ethylbis(propan-2-yl)amine (0.15 mL, 0.865 mmol) were added at 0° C. and the reaction mixture was stirred at room temperature for another 4 h. After completion the reaction mixture was concentrated and the crude residue obtained was purified by prep HPLC using water and acetonitrile (+0.1% TFA) as eluents to afford the desired product (Cpd. 13). Yield: 0.005 g, 5%; LCMS m/z 685.40 [M+H]+. 1H NMR (400 MHz, methanol-d4): δ 7.34-7.29 (m, 4H), 7.26-7.24 (m, 3H), 7.10 (d, J=8.0 Hz, 2H), 4.79 (s, 1H), 4.49 (d, J=11.2 Hz, 1H), 4.00 (s, 2H), 3.95-3.90 (m, 1H), 3.87-3.80 (m, 2H), 3.76-3.68 (m, 3H), 3.54-3.50 (m, 2H), 3.37 (dd, J=8.8 Hz & 1.2 Hz, 1H), 3.19-3.12 (m, 1H), 2.90-2.78 (m, 2H), 2.57-2.50 (m, 3H), 2.15 (t, J=2.4 Hz, 1H), 1.86-1.68 (m, 4H), 1.58-1.49 (m, 4H), 1.33-1.29 (m, 8H).

### Compound 14

### (2R,4S,5R,6R)-2-(benzyloxy)-6-((1R,2R)-3-(2-(4-(10-carboxydec-1-yn-1-yl)phenyl)acetamido)-1,2-dihydroxypropyl)-4-hydroxy-5-(2-hydroxyacetamido)tetrahydro-2H-pyran-2-carboxylic acid (Cpd. 14)

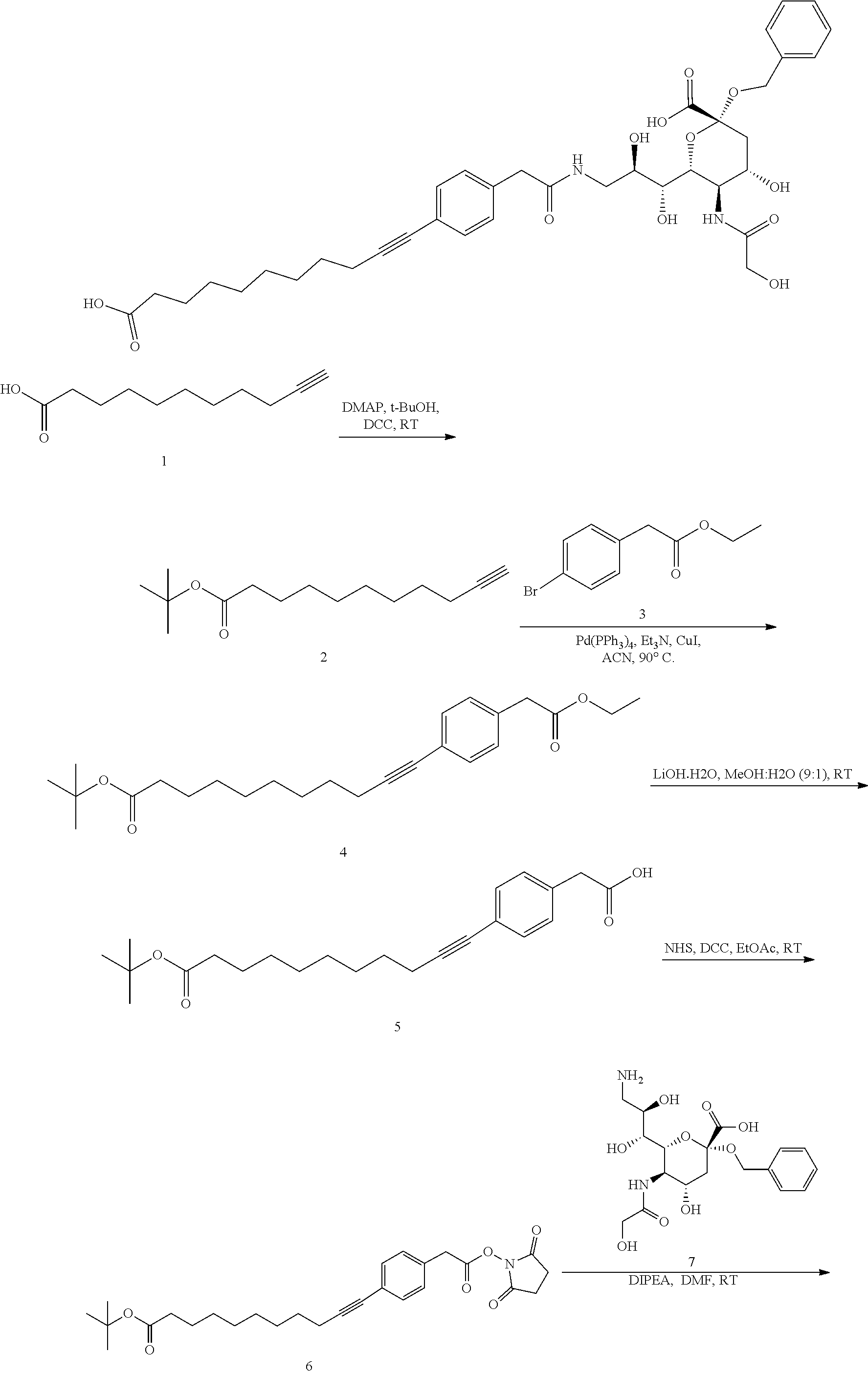

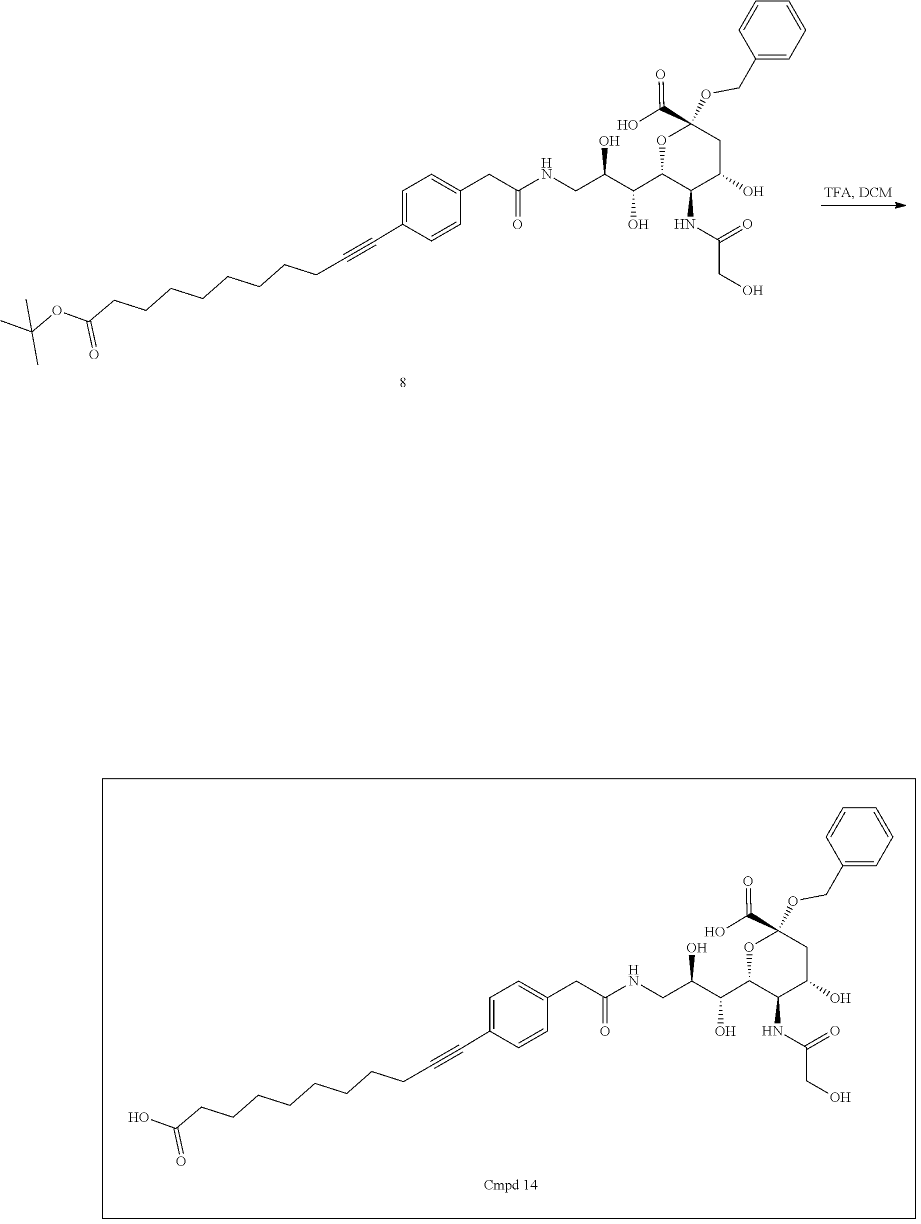 [SMILES: CC(C)(C)OC(=O)CCCCCCCCC#Cc1ccc(CC(=O)NC[C@@H](O)[C@@H](O)[C@@H]2O[C@@](OCc3ccccc3)(C(=O)O)C[C@H](O)[C@H]2NC(=O)CO)cc1 ; O=C(O)CCCCCCCCC#Cc1ccc(CC(=O)NC[C@@H](O)[C@@H](O)[C@@H]2O[C@@](OCc3ccccc3)(C(=O)O)C[C@H](O)[C@H]2NC(=O)CO)cc1]

### Synthesis of tert-butyl undec-10-ynoate (2)

To a stirred solution of undec-10-ynoic acid (1, 1.0 g, 5.49 mmol) in dichloromethane (20.0 mL), were successively added N,N-dimethylpyridin-4-amine (0.335 g, 2.74 mmol), 2-methylpropan-2-ol (1.06 mL, 11.00 mmol) and N,N′-dicyclohexylmethanediimine (1.70 g, 8.23 mmol) at 0° C. The reaction mixture was stirred for 3 h at room temperature then quenched with water and extracted with dichloromethane. The combined organic layers were dried, filtered, concentrated and the crude residue obtained was purified by silica-gel column chromatography using ethyl acetate and heptane as eluents to afford the desired product (2). Yield: 1.0 g, 76%.

### Synthesis of tert-butyl 11-(4-(2-ethoxy-2-oxoethyl)phenyl)undec-10-ynoate (4)

A mixture of tert-butyl undec-10-ynoate (2, 0.981 g, 4.11 mmol), ethyl 2-(4-bromophenyl)acetate (3, 0.50 g, 2.06 mmol) and triethylamine (0.867 mL, 6.17 mmol) in acetonitrile (10.0 mL), was degassed for 5 minutes by sparging with nitrogen gas. Tetrakis(triphenylphosphane) palladium (0.119 g, 0.103 mmol) and copper(1+) iodide (0.0196 g, 0.103 mmol) were added and the reaction mixture was stirred at 90° C. for 12 h. The reaction mixture was diluted with water and extracted with ethyl acetate. The combined organic layers was dried, filtered and concentrated. The crude residue obtained was purified by silica-gel column chromatography using ethyl acetate and heptane as eluents to afford the desired product (4). Yield: 0.40 g, 47%; LCMS (ESI) m/z 399.05 [M−H]−.

### Synthesis of 2-(4-(11-(tert-butoxy)-11-oxoundec-1-yn-1-yl)phenyl)acetic acid (5)

To a stirred solution of tert-butyl 11-(4-(2-ethoxy-2-oxoethyl)phenyl)undec-10-ynoate (4, 0.40 g, 0.979 mmol) in methanol (5.0 mL) was added a solution of lithium hydroxide monohydrate (0.032 g, 1.47 mmol) in water (1.0 mL). The reaction mixture was stirred at room temperature for 5 h then concentrated. The residue was treated with citric acid solution and extracted with ethyl acetate. The combined organic layers were dried, filtered and concentrated to afford the desired crude product (5). Yield: 0.20 g, 54%; LCMS (ESI) m/z 371.00 [M−H]−.

### Synthesis of tert-butyl 11-(4-(2-((2,5-dioxopyrrolidin-1-yl)oxy)-2-oxoethyl)phenyl)undec-10-ynoate (6)

To a stirred solution of 2-(4-(11-(tert-butoxy)-11-oxoundec-1-yn-1-yl)phenyl)acetic acid (5, 0.20 g, 0.494 mmol) in ethyl acetate (5.0 mL), 1-hydroxypyrrolidine-2,5-dione (0.171 g, 1.48 mmol) and N,N′-dicyclohexylmethanediimine (0.204 g, 0.988 mmol) was added at 0° C. and the reaction mixture was stirred at room temperature for 4 h. After completion the reaction mixture was concentrated, and the residue obtained was dissolved in ethyl acetate. The mixture was filtered, and the filtrate was washed with ethyl acetate to afford the desired crude product (6). Yield: 0.180 g, 71%; LCMS (ESI) m/z 467.95 [M−H]⁻.

### Synthesis of (2R,4S,5R,6R)-2-(benzyloxy)-6-((1R,2R)-3-(2-(4-(11-(tert-butoxy)-11-oxoundec-1-yn-1-yl)phenyl)acetamido)-1,2-dihydroxypropyl)-4-hydroxy-5-(2-hydroxyacetamido)tetrahydro-2H-pyran-2-carboxylic acid (8)

To a stirred solution of (2R,4S,5R,6R)-6-((1R,2R)-3-amino-1,2-dihydroxypropyl)-2-(benzyloxy)-4-hydroxy-5-(2-hydroxyacetamido)tetrahydro-2H-pyran-2-carboxylic acid (7, 0.220 g, 0.530 mmol) and tert-butyl 11-(4-(2-((2,5-dioxopyrrolidin-1-yl)oxy)-2-oxoethyl)phenyl)undec-10-ynoate (6, 0.249 g, 0.530 mmol) in N,N-dimethylformamide (3.0 mL) at 0° C. was added DIEA (0.46 mL, 2.65 mmol) and the reaction mixture was stirred at room temperature for 4 h. The mixture was concentrated and the crude residue obtained was triturated with diethyl ether to afford the desired crude product (8). Yield: 0.22 g; 54%; LCMS (ESI) m/z 769.30 [M+H]+;

### Synthesis of (2R,4S,5R,6R)-2-(benzyloxy)-6-((1R,2R)-3-(2-(4-(10-carboxydec-1-yn-1-yl)phenyl)acetamido)-1,2-dihydroxypropyl)-4-hydroxy-5-(2-hydroxyacetamido)tetrahydro-2H-pyran-2-carboxylic acid (Cpd. 14)

To a stirred solution of (2R,4S,5R,6R)-2-(benzyloxy)-6-((1R,2R)-3-(2-(4-(11-(tert-butoxy)-11-oxoundec-1-yn-1-yl)phenyl)acetamido)-1,2-dihydroxypropyl)-4-hydroxy-5-(2-hydroxyacetamido)tetrahydro-2H-pyran-2-carboxylic acid (8, 0.220 g, 0.286 mmol) in dichloromethane (2.0 ml) at 0° C., was added trifluoroacetic acid (1.0 mL) drop-wise. The reaction mixture was stirred for 3 h at room temperature then concentrated. The crude residue obtained was purified by prep HPLC using acetonitrile and water (+0.1% TFA) as eluents to afford the desired product (Cpd. 14). Yield: 0.056 g, 27%; LCMS (ESI) m/z 713.40 [M+H]+; 1H NMR (400 MHz, Methanol-d4): δ 7.31 (d, J=4.40 Hz, 4H), 7.29-7.21 (m, 5H), 4.80 (d, J=11.2 Hz, 2H), 4.49 (d, J=11.6 Hz, 1H), 4.02 (s, 2H), 3.94-3.77 (m, 4H), 3.66 (dd, J=13.6 Hz & 2.8 Hz, 1H), 3.52 (s, 2H), 3.40 (dd, J=8.8 Hz & 1.6 Hz, 1H), 3.27-3.23 (m, 1H), 2.78 (dd, J=12.4 Hz & 4.0 Hz, 1H), 2.38 (t, J=6.8 Hz, 2H), 2.27 (t, J=7.6 Hz, 2H), 1.80 (t, J=12.0 Hz, 1H), 1.62-1.54 (m, 4H), 1.46-1.35 (m, 8H).

### Compound 15

### (2R,4S,5R,6R)-2-(benzyloxy)-6-((1R,2R)-1,2-dihydroxy-3-(2-(4-(2-(2-(prop-2-yn-1-yloxy)ethoxy)ethoxy)phenyl)acetamido)propyl)-4-hydroxy-5-(2-hydroxyacetamido)tetrahydro-2H-pyran-2-carboxylic acid (Cpd. 15)

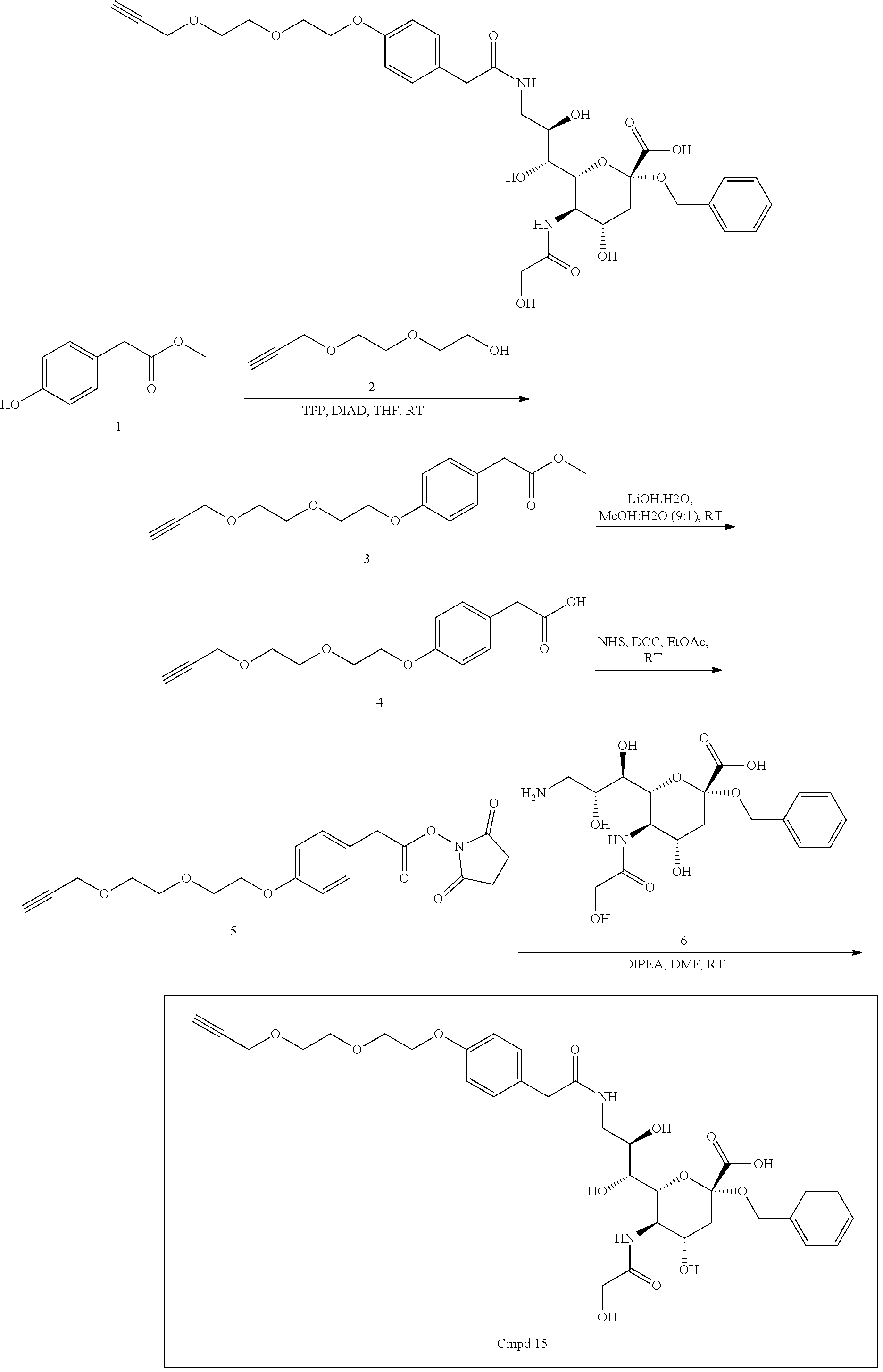

### Synthesis of methyl 2-(4-(2-(2-(prop-2-yn-1-yloxy)ethoxy)ethoxy)phenyl)acetate (3)

To a solution of methyl 2-(4-hydroxyphenyl)acetate (1, 1.0 g, 6.02 mmol) in tetrahydrofuran (10.0 mL) at 0° C. were successively added triphenylphosphine (3.16 g, 12.04 mmol), 2-(2-(prop-2-yn-1-yloxy)ethoxy)ethan-1-ol (1.64 ml, 12.04 mmol) and (E)-N-[(ethoxycarbonyl)imino]ethoxyformamide (2.38 mL, 15.04 mmol). The reaction mixture was stirred at room temperature for 12 h then diluted with water and extracted with ethyl acetate. The combined organic layers were dried, filtered and concentrated. The crude residue obtained was purified by silica-gel column chromatography using ethyl acetate and heptane as eluents to afford the desired product (3). Yield: 0.80 g, 46%; LCMS m/z 310.15 [M+H2O]+.

### Synthesis of 2-(4-(2-(2-(prop-2-yn-1-yloxy)ethoxy)ethoxy)phenyl)acetic acid (4)

To a stirred solution of methyl 2-(4-(2-(2-(prop-2-yn-1-yloxy)ethoxy)ethoxy)phenyl)acetate (3, 0.80 g, 2.74 mmol) in ethanol (8.0 mL) was added a solution of lithium hydroxide monohydrate (0.235 g, 5.48 mmol) in water (1.0 mL). The reaction mixture was stirred at room temperature for 5 h then concentrated. The residue obtained was treated with 1N HCl and the precipitate was collected by filtration, rinsed with water then diethyl ether to afford the desired product (4). Yield: 0.60 g, 79%; LCMS (ESI) m/z 277.05 [M−H]−.

### Synthesis of 2,5-dioxopyrrolidin-1-yl 2-(4-(2-(2-(prop-2-yn-1-yloxy)ethoxy)ethoxy)phenyl)acetate (5)

To a stirred solution of 2-(4-(2-(2-(prop-2-yn-1-yloxy)ethoxy)ethoxy)phenyl)acetic acid (4, 0.50 g, 1.80 mmol) in ethyl acetate (5.0 mL) at 0° C. were successively added 1-hydroxypyrrolidine-2,5-dione (0.207 g, 1.80 mmol) and N,N′-dicyclohexylmethanediimine (0.371 g, 1.80 mmol). The reaction mixture was stirred at room temperature for 4 h then concentrated under reduced pressure. The residue was taken up in ethyl acetate and the precipitate was collected by filtration, and rinsed with ethyl acetate to afford the desired product (5). Yield: 0.400 g, 59%; LCMS (ESI) m/z 393.10 [M+H2O]+.

### Synthesis of (2R,4S,5R,6R)-2-(benzyloxy)-6-((1R,2R)-1,2-dihydroxy-3-(2-(4-(2-(2-(prop-2-yn-1-yloxy)ethoxy)ethoxy)phenyl)acetamido)propyl)-4-hydroxy-5-(2-hydroxyacetamido)tetrahydro-2H-pyran-2-carboxylic acid (Cpd. 15)

To a stirred solution of (2R,4S,5R,6R)-6-((1R,2R)-3-amino-1,2-dihydroxypropyl)-2-(benzyloxy)-4-hydroxy-5-(2-hydroxyacetamido)tetrahydro-2H-pyran-2-carboxylic acid (6, 0.250 g, 0.603 mmol) and 2,5-dioxopyrrolidin-1-yl 2-(4-(2-(2-(prop-2-yn-1-yloxy)ethoxy)ethoxy)phenyl)acetate (5, 0.181 g, 0.483 mmol) in N,N-dimethylformamide (3.0 mL) at 0° C. was added DIEA (0.52 mL, 3.02 mmol) and the reaction mixture was stirred at room temperature for 4 h. After completion the reaction mixture was concentrated and the crude residue obtained was purified by prep HPLC using acetonitrile and water (+0.1% TFA) as eluents to afford the desired product (Cpd. 15). Yield: 0.078 g; 19%; LCMS (ESI) m/z 675.50 [M+H]+. 1H NMR (400 MHz, MeOD-d4): δ 7.92 (d, J=8.0 Hz, 1H), 7.32-7.25 (m, 5H), 7.22-7.18 (m, 2H), 6.88-6.84 (dt, J=9.6 Hz & 2.8 Hz, 2H), 4.79 (d, J=11.2 Hz, 1H), 4.48 (d, J=11.6 Hz, 1H), 4.18 (d, J=2.4 Hz, 2H), 4.07-4.04 (m, 2H), 4.02 (s, 2H), 3.92-3.83 (m, 3H), 3.81-3.76 (m, 3H), 3.71-3.63 (m, 5H), 3.46 (s, 2H), 3.39 (dd, J=8.8 Hz & 1.6 Hz, 1H), 3.28-3.22 (m, 1H), 2.84 (t, J=2.4 Hz, 1H), 2.77 (dd, J=12.8 Hz & 4.0 Hz, 1H), 1.80 (t, J=12.4 Hz, 1H).

### Compound 16

### (2R,4S,5R,6R)-6-((1R,2R)-1,2-dihydroxy-3-(2-(4-(1-(12-oxo-3,6,9-trioxa-13-azapentadecyl)-1H-1,2,3-triazol-4-yl)phenyl)acetamido)propyl)-2-(hexyloxy)-4-hydroxy-5-(2-hydroxyacetamido)tetrahydro-2H-pyran-2-carboxylic acid (Cpd. 16)

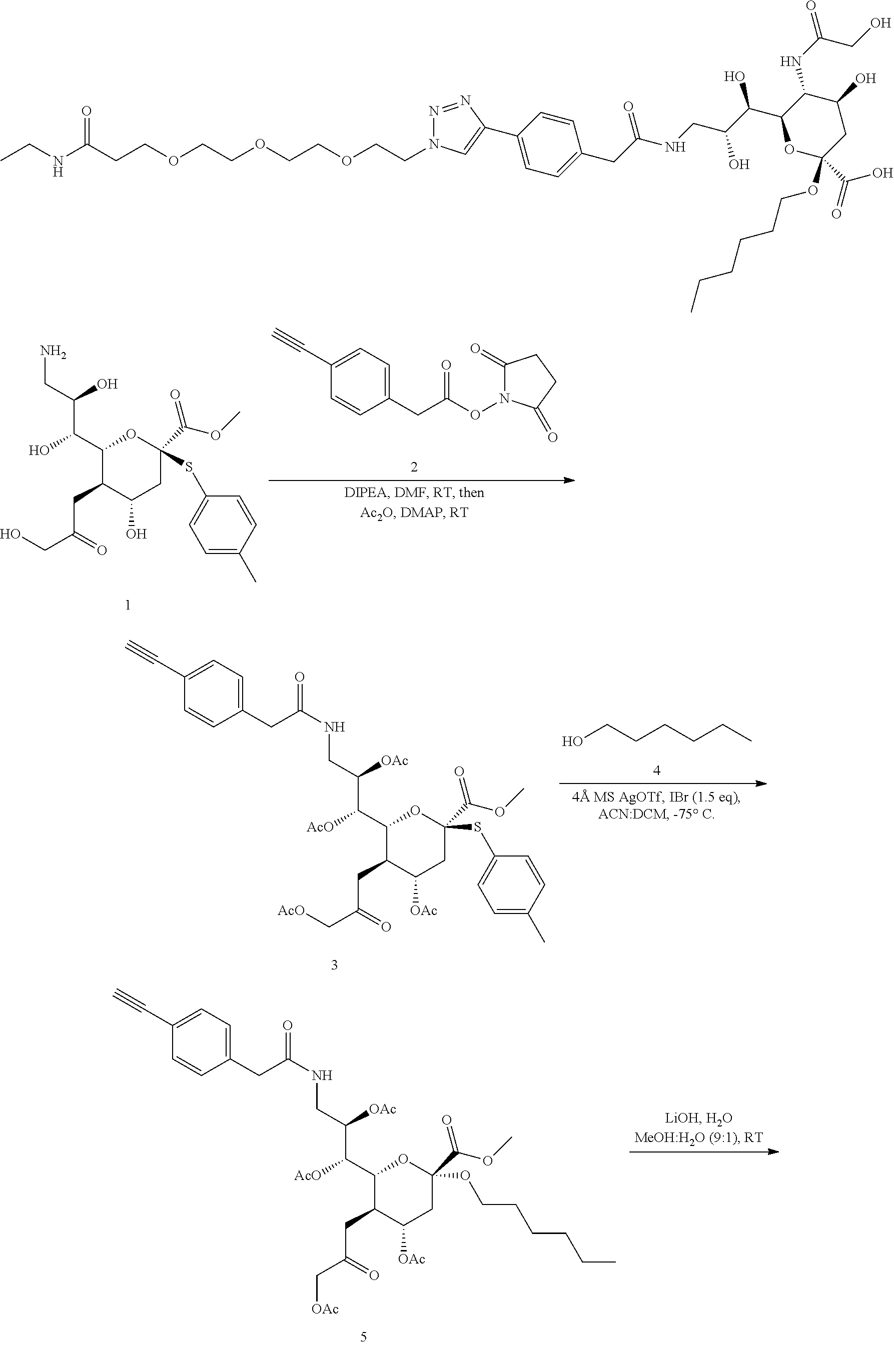

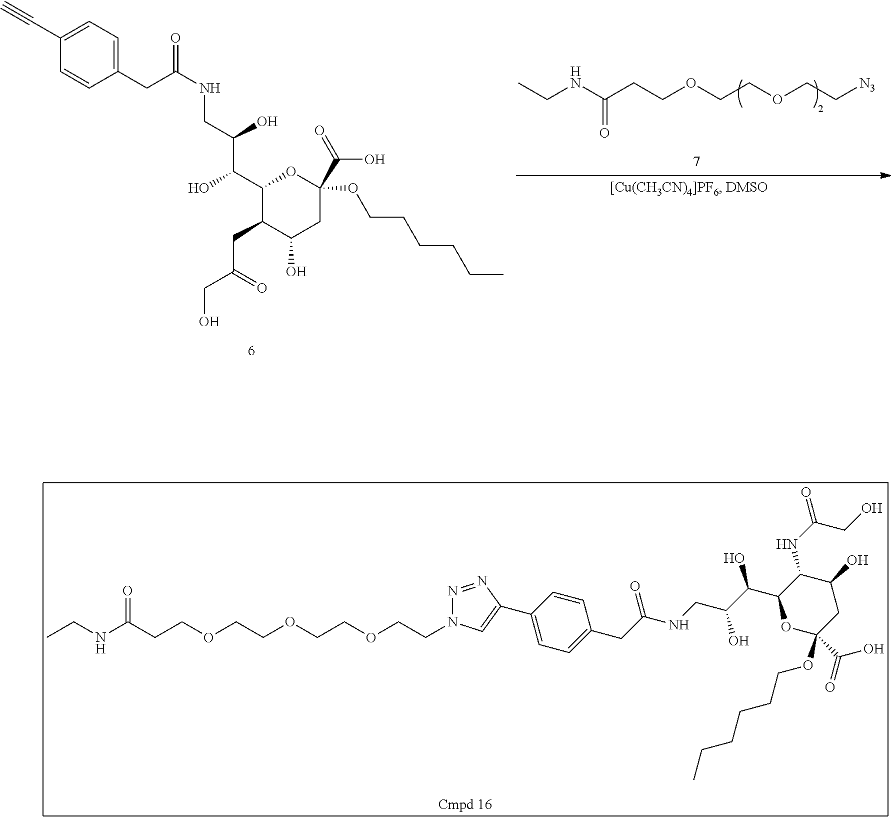

### Synthesis of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-ethynylphenyl)acetamido)propane-1,2-diyl diacetate (3)

To a mixture of methyl (2R,4S,5R,6R)-6-((1R,2R)-3-amino-1,2-dihydroxypropyl)-4-hydroxy-5-(2-hydroxyacetamido)-2-(p-tolylthio)tetrahydro-2H-pyran-2-carboxylate (1, 4.0 g, 9.00 mmol) and 2,5-dioxopyrrolidin-1-yl 2-(4-ethynylphenyl)acetate (2, 1.85 g, 7.20 mmol) in dimethylformamide (40.0 mL) at 0° C. was added DIEA (15.7 mL, 90.00 mmol), and the reaction mixture was stirred at room temperature for 4 h. DMAP (0.550 g, 4.50 mmol) and acetyl acetate (8.51 mL, 90.0 mmol) were added and the mixture was stirred at room temperature for another 12 h. After completion the mixture was diluted with water and ethyl acetate. The organic layer was separated, dried, filtered and concentrated. The crude residue obtained was purified by silica-gel column chromatography using ethyl acetate and heptane as eluents to afford the desired product (3). Yield: 2.20 g, 33%; LCMS (ESI) m/z 754.75 [M+H]⁺.

### Synthesis of (1R,2R)-1-((2R,3R,4S,6R)-4-acetoxy-3-(2-acetoxyacetamido)-6-(hexyloxy)-6-(methoxycarbonyl)tetrahydro-2H-pyran-2-yl)-3-(2-(4-ethynylphenyl)acetamido)propane-1,2-diyl diacetate (5)

A mixture of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-ethynylphenyl)acetamido)propane-1,2-diyl diacetate (3, 0.50 g, 0.662 mmol), hexan-1-ol (4, 0.203 g, 1.99 mmol), silver(1+) trifluoromethanesulfonate (0.340 g, 1.32 mmol) and activated 4 Å powdered molecular sieves (1.00 g) in anhydrous dichloromethane (18.0 mL) and anhydrous acetonitrile (30.0 mL) was stirred at room temperature for 1 h. The mixture was cooled to −78° C. and a solution of iodobromane (0.205 g, 0.993 mmol) in dichloromethane (2.0 mL) was added dropwise. The reaction mixture was stirred at −78° C. temperature for 2 h then quenched by dropwise addition of triethylamine (1.0 mL) and warmed to room temperature. The reaction mixture was filtered, the filtrate was washed with saturated solution of sodium bicarbonate, dried, filtered and concentrated. The crude residue obtained was purified by silica-gel column chromatography using ethyl acetate and heptane as eluents to afford the desired product (5). Yield: 0.350 g, 72%; LCMS m/z 730.90 [M−H]⁻.

### Synthesis of (2R,4S,5R,6R)-6-((1R,2R)-3-(2-(4-ethynylphenyl)acetamido)-1,2-dihydroxypropyl)-2-(hexyloxy)-4-hydroxy-5-(2-hydroxyacetamido)tetrahydro-2H-pyran-2-carboxylic acid (6)

To a stirred solution of (1R,2R)-1-((2R,3R,4S,6R)-4-acetoxy-3-(2-acetoxyacetamido)-6-(hexyloxy)-6-(methoxycarbonyl)tetrahydro-2H-pyran-2-yl)-3-(2-(4-ethynylphenyl)acetamido)propane-1,2-diyl diacetate (5, 0.250 g, 0.341 mmol) in methanol (3.0 mL) and water (0.50 mL) was added lithium hydroxide monohydrate (0.087 g, 2.04 mmol) at 0° C. The reaction mixture was stirred at room temperature for 6 h then treated with acidic (Dowex 50, H⁺) to pH˜6. The suspension was filtered, the filtrate was concentrated and the crude residue obtained was purified by prep HPLC using acetonitrile and water (+0.1% TFA) as eluents to afford the desired product (6). Yield: 0.022 g, 12%; LCMS m/z 551.40 [M+H]⁺.

### Synthesis of (2R,4S,5R,6R)-6-((1R,2R)-1,2-dihydroxy-3-(2-(4-(1-(12-oxo-3,6,9-trioxa-13-azapentadecyl)-1H-1,2,3-triazol-4-yl)phenyl)acetamido)propyl)-2-(hexyloxy)-4-hydroxy-5-(2-hydroxyacetamido)tetrahydro-2H-pyran-2-carboxylic acid (Cpd. 16)

To a stirred solution of (2R,4S,5R,6R)-6-((1R,2R)-3-(2-(4-ethynylphenyl)acetamido)-1,2-dihydroxypropyl)-2-(hexyloxy)-4-hydroxy-5-(2-hydroxyacetamido)tetrahydro-2H-pyran-2-carboxylic acid (6, 0.020 g, 0.036 mmol) and 3-(2-(2-(2-azidoethoxy)ethoxy)ethoxy)-N-ethylpropanamide (0.012 g, 0.043 mmol) in dimethyl sulfoxide (1.0 mL) was added tetrakis(acetonitrile)copper(I) hexafluorophosphate (0.037 g, 0.102 mmol) and reaction mixture was stirred at room temperature for 15 minutes. After completion, acetic acid (0.1 mL) was added and the resulting solution was purified by prep HPLC using acetonitrile and water (+0.1% TFA) as eluents to afford the desired product (Cpd. 16). Yield: 0.012 g, 40%; LCMS (ESI) m/z 825.60 [M+H]⁺; ¹H NMR (400 MHz, methanol-d₄): δ8.35 (s, 1H), 7.77 (d, J=8.0 Hz, 2H), 7.39 (d, J=8.4 Hz, 2H), 4.62 (t, J=4.8 Hz, 2H), 4.00 (s, 2H), 3.94-3.91 (m, 2H), 3.89-3.66 (m, 6H), 3.64-3.56 (m, 8H), 3.56-3.48 (m, 4H), 3.41-3.35 (m, 2H), 3.28-3.25 (m, 1H), 3.19-3.12 (m, 2H), 2.71 (dd, J=12.4 Hz & 4.64 Hz, 1H), 2.35 (t, J=6.0 Hz, 1H), 1.72 (t, J=12.4 Hz, 1H), 1.55-1.48 (m, 2H), 1.32-1.25 (m, 6H), 1.07 (t, J=7.2 Hz, 3H), 0.89 (t, J=6.8 Hz, 3H).

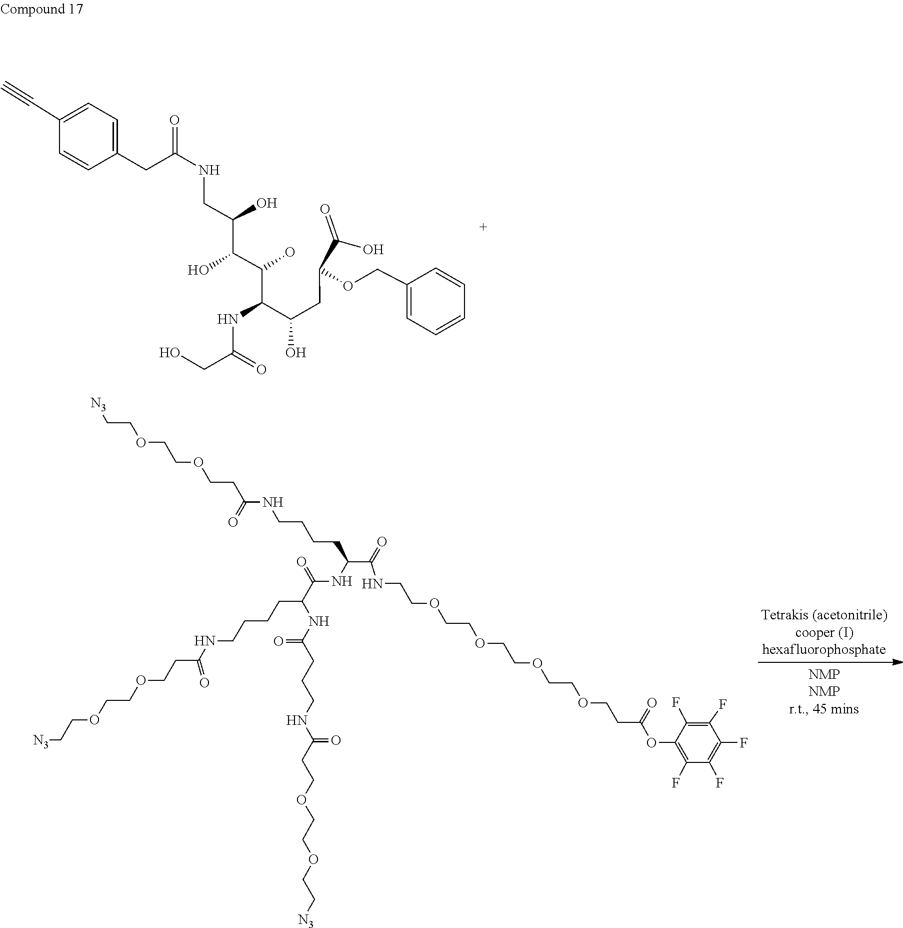

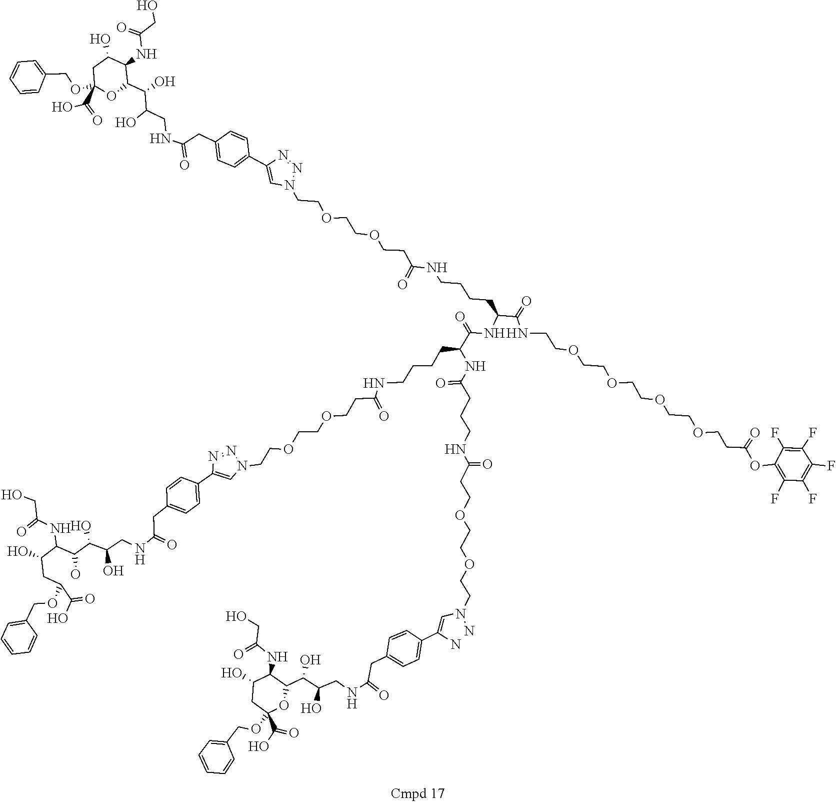

To a solution of alkyne (43.6 mg, 78.3 umol) in anhydrous NMP (245 uL) was added tri-azide (32.5 mg, 24.5 umol) in anhydrous NMP (245 uL), followed by Tetrakis(acetonitrile) copper(l) hexafluorophosphate (41.0 mg, 110 umol). The vial was closed and the mixture was stirred for 1 h at room temperature, then filtered and the filtrate was purified by reverse phase column chromatography using acetonitrile and water as eluents (+0.1% TFA) to afford the desired product (Cpd. 17). Yield: 40.6 mg; 55%. LCMS m/z 1065 [M+H]+.

### Compound 18

### (2R,4S,5R,6R)-2-(benzyloxy)-6-((1R,2R)-3-(2-(4-chlorophenyl)acetamido)-1,2-dihydroxypropyl)-5-(2-hydroxyacetamido)-4-(prop-2-yn-1-yloxy)tetrahydro-2H-pyran-2-carboxylic acid (Cpd. 18)

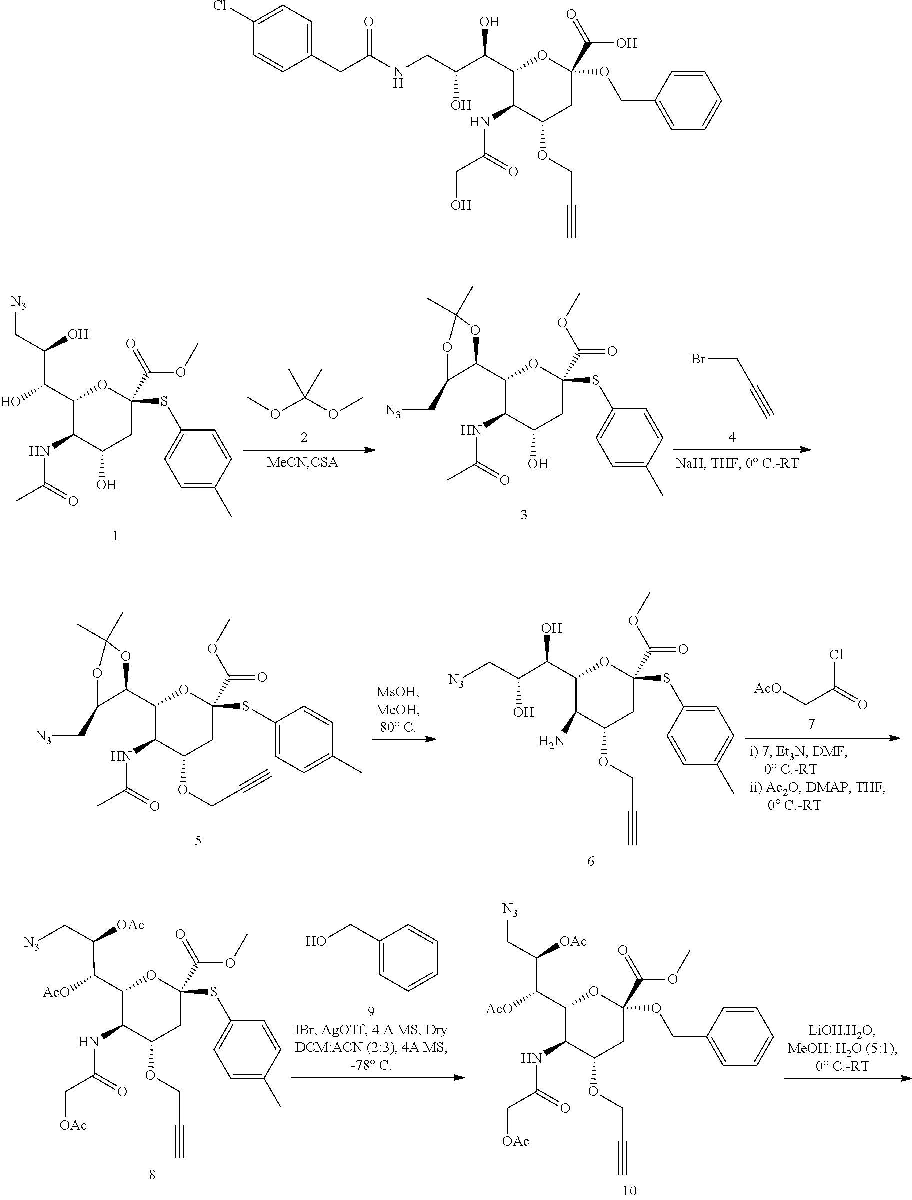

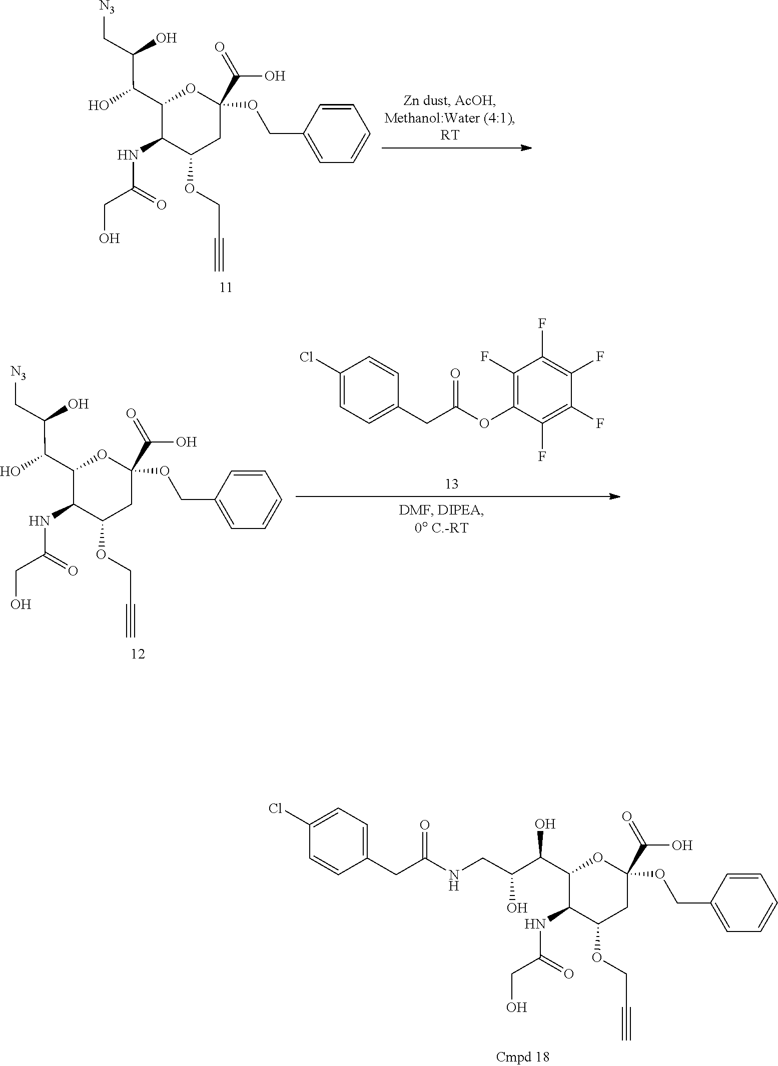 [SMILES: C#CCO[C@H]1C[C@](OCc2ccccc2)(C(=O)O)O[C@@H]([C@H](O)[C@H](O)CN=[N+]=[N-])[C@@H]1NC(=O)CO ; C#CCO[C@H]1C[C@](OCc2ccccc2)(C(=O)O)O[C@@H]([C@H](O)[C@H](O)CN=[N+]=[N-])[C@@H]1NC(=O)CO ; O=C(Cc1ccc(Cl)cc1)Oc1c(F)c(F)c(F)c(F)c1F ; C#CCO[C@H]1C[C@](OCc2ccccc2)(C(=O)O)O[C@@H]([C@H](O)[C@H](O)CNC(=O)Cc2ccc(Cl)cc2)[C@@H]1NC(=O)CO]

### Synthesis of methyl (2R,4S,5R,6R)-5-acetamido-6-((4R,5R)-5-(azidomethyl)-2,2-dimethyl-1,3-dioxolan-4-yl)-4-hydroxy-2-(p-tolylthio)tetrahydro-2H-pyran-2-carboxylate (3)

To a 250 mL round bottom flask charged with methyl (2R,4S,5R,6R)-5-acetamido-6-((1R,2R)-3-azido-1,2-dihydroxypropyl)-4-hydroxy-2-(p-tolylthio)tetrahydro-2H-pyran-2-carboxylate (1, 7.5 g, 16.5 mmol) in acetonitrile (72.7 mL, 1.39 mol) were added camphorsulfonic acid (0.38 g, 1.65 mmol) and 2,2-dimethoxypropane (2, 6.87 g, 66.0 mmol) at 0° C. and the reaction mixture was stirred at room temperature for 2 h. After completion, the reaction mixture was concentrated, diluted with cold water and extracted with ethyl acetate (3×100 mL). The combined organic layers were dried, filtered, concentrated, and the crude residue obtained was purified by silica-gel column chromatography using methanol and DCM as eluents to afford the desired product (3). Yield: 7.5 g, 91.90%; LCMS (ESI) m/z 454.90 [M+H]⁺.

### Synthesis of methyl (2R,4S,5R,6R)-5-acetamido-6-((4R,5R)-5-(azidomethyl)-2,2-dimethyl-1,3-dioxolan-4-yl)-4-(prop-2-yn-1-yloxy)-2-(p-tolylthio)tetrahydro-2H-pyran-2-carboxylate (5)

To a stirred solution of methyl (2R,4S,5R,6R)-5-acetamido-6-((4R,5R)-5-(azidomethyl)-2,2-dimethyl-1,3-dioxolan-4-yl)-4-hydroxy-2-(p-tolylthio)tetrahydro-2H-pyran-2-carboxylate (3, 5.0 g, 10.10 mmol) in THF (50 mL) was added sodium hydride (0.73 g, 30.30 mmol) portion wise at 0° C. After 10 mins of stirring at 0° C., 3-bromopropyne (4, 1.53 mL, 20.20 mmol) was added drop-wise at 0° C. The reaction mixture was stirred at room temperature for 2 h then diluted with ethyl acetate, and carefully quenched with ice cold water and ammonium chloride solution. The organic layer was separated, dried, filtered, then concentrated. The crude residue obtained was purified by silica-gel column chromatography using ethyla acetate and heptane as eluents to afford the desired product (5). Yield: 3.0 g, 55.71%; LCMS (ESI) m/z 530.70 [M−H]⁻.

### Synthesis of methyl (2R,4S,5R,6R)-5-amino-6-((1R,2R)-3-azido-1,2-dihydroxypropyl)-4-(prop-2-yn-1-yloxy)-2-(p-tolylthio)tetrahydro-2H-pyran-2-carboxylate (6)

To a stirred solution of methyl (2R,4S,5R,6R)-5-acetamido-6-((4R,5R)-5-(azidomethyl)-2,2-dimethyl-1,3-dioxolan-4-yl)-4-(prop-2-yn-1-yloxy)-2-(p-tolylthio)tetrahydro-2H-pyran-2-carboxylate (5, 2.50 g, 4.69 mmol) in methanol (50.0 mL), was added methane sulfonic acid (3.05 mL, 46.90 mmol) dropwise at 0° C. The reaction mixture was stirred at 80° C. for 30 h then concentrated and triturated with 10% ethyl acetate in heptane (4-5 times) to afford the desired crude product (6). Yield: 2.0 g; LC-MS (ESI) m/z 451.05 [M+H]⁺.

### Synthesis of (1R,2R)-1-((2R,3R,4S,6R)-3-(2-acetoxyacetamido)-6-(methoxycarbonyl)-4-(prop-2-yn-1-yloxy)-6-(p-tolylthio)tetrahydro-2H-pyran-2-yl)-3-azidopropane-1,2-diyl diacetate (8)

To a stirred solution of methyl (2R,4S,5R,6R)-5-amino-6-((1R,2R)-3-azido-1,2-dihydroxypropyl)-4-(prop-2-yn-1-yloxy)-2-(p-tolylthio)tetrahydro-2H-pyran-2-carboxylate (6, 3.5 g, 7.77 mmol) in dry N,N-dimethylformamide (10.0 mL) at 0° C. was added triethylamine (10.8 mL, 77.70 mmol), followed by 2-chloro-2-oxoethyl acetate (7, 1.25 mL, 11.70 mmol) in dry N,N-dimethylformamide (2.0 mL) drop wise at 0° C. The reaction mixture was stirred from 0° C. to room temperature over 2 h. 4-dimethylaminopyridine (854 mg, 6.99 mmol) was added, followed by acetic anhydride (6.01 mL, 63.6 mmol) dropwise. The reaction was stirred at room temperature for 16 h then diluted with ethyl acetate and washed with ice cold water. The organic layer was dried, filtered and the filtrate was concentrated. The crude residue obtained was purified by silica-gel column chromatography using ethyl acetate in heptane to afford the desired product (8). Yield: 1.5 g, 30.42% (over two steps); LCMS (ESI) m/z 635.00 [M+H]⁺.

### Synthesis of (1R,2R)-1-((2R,3R,4S,6R)-3-(2-acetoxyacetamido)-6-(benzyloxy)-6-(methoxycarbonyl)-4-(prop-2-yn-1-yloxy)tetrahydro-2H-pyran-2-yl)-3-azidopropane-1,2-diyl diacetate (10)

A mixture of (1R,2R)-1-((2R,3R,4S,6R)-3-(2-acetoxyacetamido)-6-(methoxycarbonyl)-4-(prop-2-yn-1-yloxy)-6-(p-tolylthio)tetrahydro-2H-pyran-2-yl)-3-azidopropane-1,2-diyl diacetate (8, 1.10 g, 1.73 mmol), phenylmethanol (9, 1.26 mL, 12.10 mmol), silver(1+) trifluoromethanesulfonate (1.34 g, 5.20 mmol) and activated 4 Å powdered molecular sieves (1.00 g) in anhydrous dichloromethane (55.0 mL) and anhydrous acetonitrile (90.0 mL) was stirred at room temperature for 1 h then cooled to −78° C. A solution of iodobromane (0.71 g, 3.47 mmol) in dichloromethane (5.0 mL) was added dropwise and the mixture was stirred at this temperature for 2 h. The reaction mixture was quenched by triethylamine (1.0 mL) and warmed to room temperature and filtered. The filtrate was washed by a saturated solution of sodium bicarbonate and dried over sodium sulfate, filtered and concentrated under reduced pressure. The crude residue obtained was purified by silica-gel column chromatography using ethyl acetate and heptane as eluents to afford (10). Yield: 0.50 g, 46.64%; LCMS m/z 616.90 [M+H]⁺.

### Synthesis of (2R,4S,5R,6R)-6-((1R,2R)-3-azido-1,2-dihydroxypropyl)-2-(benzyloxy)-5-(2-hydroxyacetamido)-4-(prop-2-yn-1-yloxy)tetrahydro-2H-pyran-2-carboxylic acid (11)

To a stirred solution of (1R,2R)-1-((2R,3R,4S,6R)-3-(2-acetoxyacetamido)-6-(benzyloxy)-6-(methoxycarbonyl)-4-(prop-2-yn-1-yloxy)tetrahydro-2H-pyran-2-yl)-3-azidopropane-1,2-diyl diacetate (10, 0.70 g, 1.13 mmol) in methanol (10.0 mL) and water (1.0 mL) was added lithium hydroxide monohydrate (0.16 g, 6.79 mmol) at 0° C. The reaction mixture was stirred at room temperature for 4 h then neutralized with acidic (Dowex 50, H⁺) to pH˜6. The suspension was filtered, and the filtrate was concentrated. The crude residue obtained was purified by trituration to afford the desired crude product (11). Yield: 0.50 g, 80%; LCMS m/z 479.00 [M+H]⁺.

### Synthesis of (2R,4S,5R,6R)-6-((1R,2R)-3-amino-1,2-dihydroxypropyl)-2-(benzyloxy)-5-(2-hydroxyacetamido)-4-(prop-2-yn-1-yloxy)tetrahydro-2H-pyran-2-carboxylic acid (12)

To a stirred solution of (2R,4S,5R,6R)-6-((1R,2R)-3-azido-1,2-dihydroxypropyl)-2-(benzyloxy)-5-(2-hydroxyacetamido)-4-(prop-2-yn-1-yloxy)tetrahydro-2H-pyran-2-carboxylic acid (11, 0.30 g, 0.63 mmol) in methanol (8.0 mL) and water (2.0 mL), were successively added zinc dust (0.41 g, 6.27 mmol) and acetic acid (0.18 mL, 3.14 mmol) drop-wise at 0° C. The reaction mixture was stirred at room temperature for 2 h then filtered. The filtrate was concentrated and the crude residue obtained was purified by trituration with diethyl ether to afford the desired crude product (12). Yield: 0.30 g, 74%; LCMS m/z 452.85 [M+H]⁺.

### Synthesis of (2R,4S,5R,6R)-2-(benzyloxy)-6-((1R,2R)-3-(2-(4-chlorophenyl)acetamido)-1,2-dihydroxypropyl)-5-(2-hydroxyacetamido)-4-(prop-2-yn-1-yloxy)tetrahydro-2H-pyran-2-carboxylic acid (Cpd. 18)

To a stirred solution of (2R,4S,5R,6R)-6-((1R,2R)-3-amino-1,2-dihydroxypropyl)-2-(benzyloxy)-5-(2-hydroxyacetamido)-4-(prop-2-yn-1-yloxy)tetrahydro-2H-pyran-2-carboxylic acid (12, 0.30 g, 0.66 mmol) in N,N-dimethylformamide (3.0 mL), was added N,N-diisopropylethylamine (0.57 mL, 3.32 mmol) followed by perfluorophenyl 2-(4-chlorophenyl)acetate (13, 0.18 g, 0.53 mmol) at 0° C. The reaction mixture was stirred at room temperature for 4 h then concentrated. The crude residue obtained was purified by prep HPLC using water and acetonitrile as eluents to afford the desired product (Cpd. 18). Yield: 0.09 g, 22.43%; LCMS (ESI) m/z 605.30 [M+H]⁺. ¹H NMR (400 MHz, Methanol-d₄): δ 7.33-7.26 (m, 9H), 4.80 (d, J=11.2 Hz, 1H), 4.50 (d, J=11.2 Hz, 1H), 4.24 (d, J=2.4 Hz, 2H), 4.03 (s, 2H), 3.97-3.87 (m, 4H), 3.66 (dd, J=13.6 & 2.8 Hz, 1H), 3.52 (s, 2H), 3.40 (dd, J=8.8 & 1.2 Hz, 1H), 3.31-3.28 (m, 1H), 2.99 (dd, J=12.4 & 4.0 Hz, 1H), 2.85 (t, J=2.4 Hz, 1H), 1.78-1.72 (m, 1H).

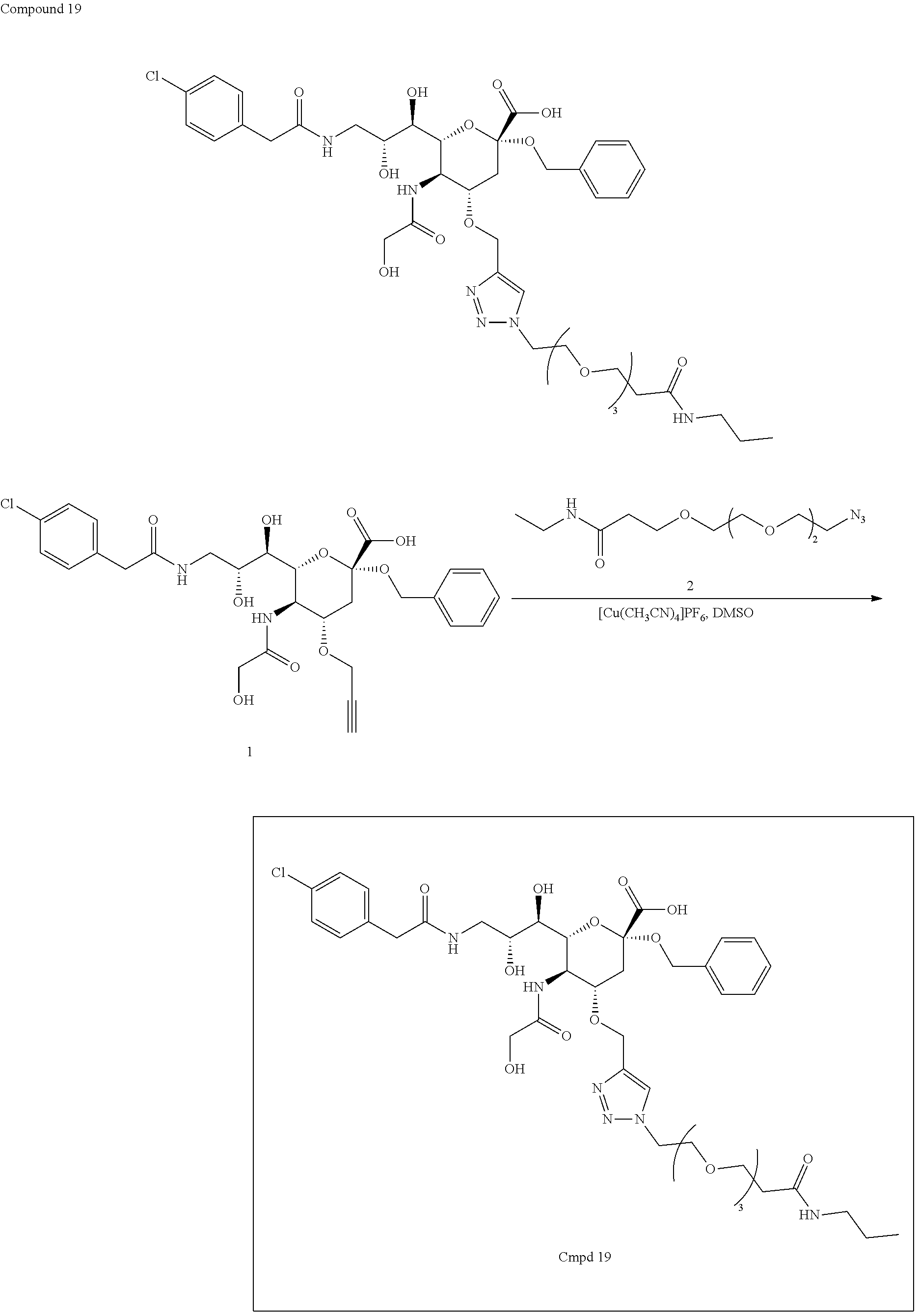

### Synthesis of (2R,4S,5R,6R)-2-(benzyloxy)-6-((1R,2R)-3-(2-(4-chlorophenyl)acetamido)-1,2-dihydroxypropyl)-5-(2-hydroxyacetamido)-4-((1-(12-oxo-3,6,9-trioxa-13-azahexadecyl)-1H-1,2,3-triazol-4-yl)methoxy)tetrahydro-2H-pyran-2-carboxylic acid (Cpd. 19)

To a stirred solution of (2R,4S,5R,6R)-2-(benzyloxy)-6-((1R,2R)-3-(2-(4-chlorophenyl)acetamido)-1,2-dihydroxypropyl)-5-(2-hydroxyacetamido)-4-(prop-2-yn-1-yloxy)tetrahydro-2H-pyran-2-carboxylic acid (1, 0.04 g, 0.066 mmol), 3-(2-(2-(2-azidoethoxy)ethoxy)ethoxy)-N-propylpropanamide (2, 0.38 g, 0.13 mmol) in dimethyl sulfoxide (1 mL), was added λ¹-copper(1+) tetrakis(acetonitrile) hexafluoride λ⁻⁵-phosphanepentauide (0.069 g, 0.18 mmol) and the reaction mixture was stirred at room temperature for 15 minutes. Progress of the reaction was monitored by LCMS and after completion, acetic acid (0.1 mL) was added to the reaction mixture. The resulting solution was diluted with acetonitrile and purified by prep HPLC (0-100% acetonitrile in water with 0.1% TFA). Fractions containing the desired product were combined and lyophilized to afford (2R,4S,5R,6R)-2-(benzyloxy)-6-((1R,2R)-3-(2-(4-chlorophenyl)acetamido)-1,2-dihydroxypropyl)-5-(2-hydroxyacetamido)-4-((1-(12-oxo-3,6,9-trioxa-13-azahexadecyl)-1H-1,2,3-triazol-4-yl)methoxy)tetrahydro-2H-pyran-2-carboxylic acid (Cpd. 19) as an off-white solid. Yield: 0.027 g, 45.71%; LCMS m/z 893.45 [M+1]⁺; ¹H-NMR (400 MHz, Methanol-d₄): δ 7.98 (s, 1H), 7.32-7.27 (m, 9H), 4.86-4.74 (m, 2H), 4.65 (d, J=12.4 Hz, 1H), 4.57 (t, J=4.8 Hz, 2H), 4.50 (d, J=11.2 Hz, 1H), 3.99-3.83 (m, 8H), 3.69 (t, J=6.0 Hz, 3H), 3.58-3.52 (m, 10H), 3.40 (d, J=8.8 Hz, 1H), 3.27-3.23 (m, 2H), 3.11 (t, J=7.2 Hz, 2H), 2.88 (dd, J=12.0 & 8.0 Hz, 1H), 2.41 (t, J=6.0 Hz, 2H), 1.77 (t, J=12.0 Hz, 1H), 1.52-1.47 (m, 2H), 0.90 (t, J=7.6 Hz, 3H).

### Compound 20

### (2R,4S,5R,6R)-2-((5-carboxypentyl)oxy)-6-((1R,2R)-3-(2-(4-chlorophenyl)acetamido)-1,2-dihydroxypropyl)-5-(2-hydroxyacetamido)-4-(prop-2-yn-1-yloxy)tetrahydro-2H-pyran-2-carboxylic acid (Cpd. 20)

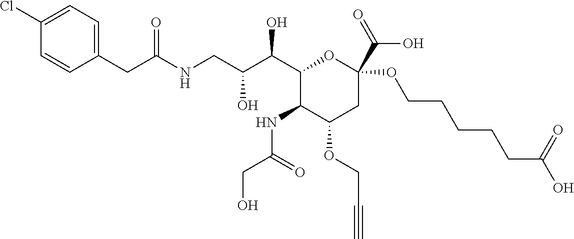 [SMILES: C#CCO[C@H]1C[C@](OCCCCCC(=O)O)(C(=O)O)O[C@@H]([C@H](O)[C@H](O)CNC(=O)Cc2ccc(Cl)cc2)[C@@H]1NC(=O)CO]

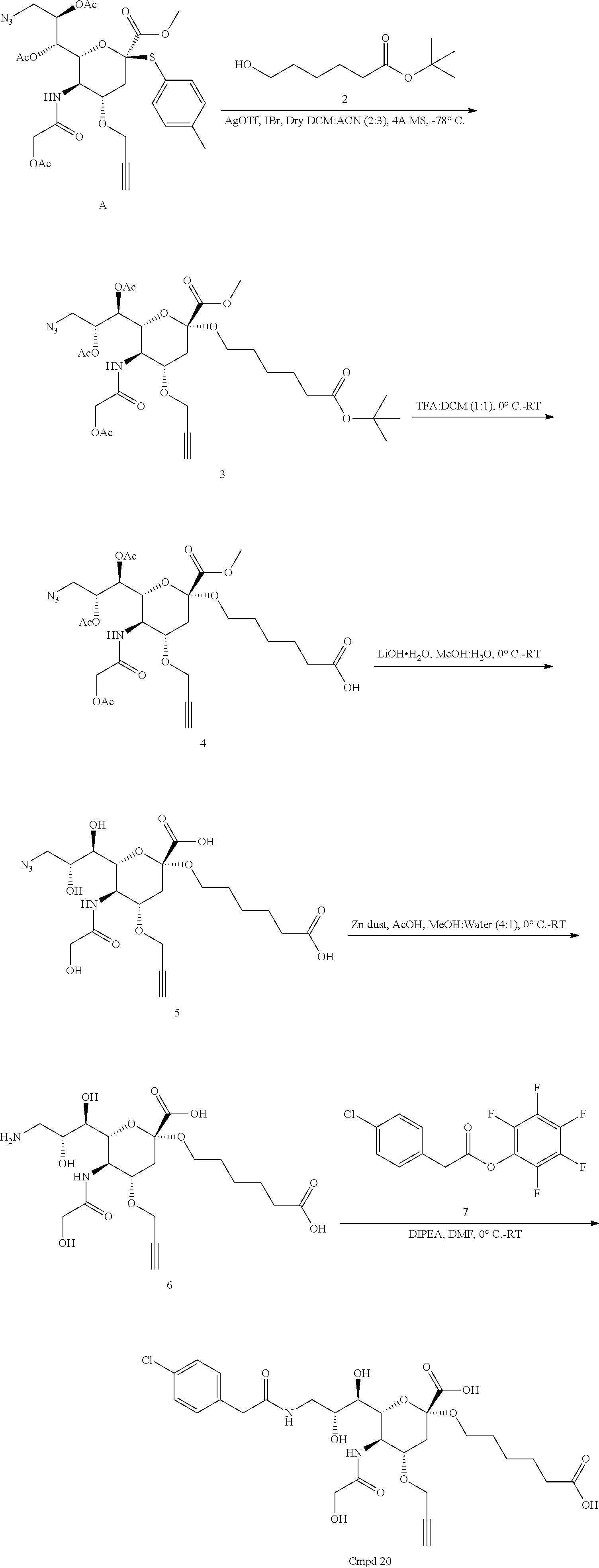

### Synthesis of (1R,2R)-1-((2R,3R,4S,6R)-3-(2-acetoxyacetamido)-6-((6-(tert-butoxy)-6-oxohexyl)oxy)-6-(methoxycarbonyl)-4-(prop-2-yn-1-yloxy)tetrahydro-2H-pyran-2-yl)-3-azidopropane-1,2-diyl diacetate (3)

A mixture of (1R,2R)-1-((2R,3R,4S,6R)-3-(2-acetoxyacetamido)-6-(methoxycarbonyl)-4-(prop-2-yn-1-yloxy)-6-(p-tolylthio)tetrahydro-2H-pyran-2-yl)-3-azidopropane-1,2-diyl diacetate (1, 1.30 g, 2.05 mmol), tert-butyl 6-hydroxyhexanoate (2, 3.47 g, 18.40 mmol), silver(1+) trifluoromethanesulfonate (1.58 g, 6.14 mmol) and activated 4 Å powdered molecular sieves (1.00 g) in anhydrous dichloromethane (55.0 mL) and anhydrous acetonitrile (90.0 mL) was stirred at room temperature for 1 h. The mixture was cooled to −78° C. and a solution of iodobromane (0.85 g, 4.01 mmol) in dichloromethane (5.0 mL) was added dropwise. The reaction mixture was stirred at −78° C. temperature for 2 h then quenched by dropwise addition of triethylamine (1.0 mL) and warmed to room temperature. The reaction mixture was filtered, the filtrate was washed with saturated solution of sodium bicarbonate, dried, filtered and concentrated. The crude residue obtained was purified by silica-gel column chromatography using ethyl acetate and heptane as eluents to afford the desired product (3). Yield: 0.90 g, 62.88%; LCMS m/z 716.75 [M+H₂O]+.

### Synthesis of 6-(((2R,4S,5R,6R)-5-(2-acetoxyacetamido)-6-((1R,2R)-1,2-diacetoxy-3-azidopropyl)-2-(methoxycarbonyl)-4-(prop-2-yn-1-yloxy)tetrahydro-2H-pyran-2-yl)oxy)hexanoic acid (4)

To a stirred solution of (1R,2R)-1-((2R,3R,4S,6R)-3-(2-acetoxyacetamido)-6-((6-(tert-butoxy)-6-oxohexyl)oxy)-6-(methoxycarbonyl)-4-(prop-2-yn-1-yloxy)tetrahydro-2H-pyran-2-yl)-3-azidopropane-1,2-diyl diacetate (3, 0.10 g, 0.14 mmol), in anhydrous dichloromethane (3.0 mL), was added trifluoroacetic acid (1.0 mL) at 0° C. and the reaction mixture was stirred at room temperature for 3 h. The solvent was concentrated to afford the desired crude product (4) which was used for the next step without further purification. Yield: 0.10 g, Crude; LCMS (ESI) m/z 642.75 [M+H]+.

### Synthesis of (2R,4S,5R,6R)-6-((1R,2R)-3-azido-1,2-dihydroxypropyl)-2-((5-carboxypentyl)oxy)-5-(2-hydroxyacetamido)-4-(prop-2-yn-1-yloxy)tetrahydro-2H-pyran-2-carboxylic acid (5)

To a stirred solution of 6-(((2R,4S,5R,6R)-5-(2-acetoxyacetamido)-6-((1R,2R)-1,2-diacetoxy-3-azidopropyl)-2-(methoxycarbonyl)-4-(prop-2-yn-1-yloxy)tetrahydro-2H-pyran-2-yl)oxy)hexanoic acid (4, 0.70 g, 1.09 mmol) in methanol (10.0 mL) and water (1.0 mL) was added lithium hydroxide monohydrate (0.18 g, 7.63 mmol) at 0° C. The reaction mixture was stirred at room temperature for 4 h then treated with acidic resin (Dowex 50, H+) to pH˜6. The suspension was filtered, the filtrate was concentrated, and the crude residue obtained was purified by trituration to afford the desired crude product (5). Yield: 0.50 g, 91.35%; LCMS m/z 501.15 [M−H]−.

### Synthesis of (2R,4S,5R,6R)-6-((1R,2R)-3-amino-1,2-dihydroxypropyl)-2-((5-carboxypentyl)oxy)-5-(2-hydroxyacetamido)-4-(prop-2-yn-1-yloxy)tetrahydro-2H-pyran-2-carboxylic acid (6)

To a stirred solution of (2R,4S,5R,6R)-6-((1R,2R)-3-azido-1,2-dihydroxypropyl)-2-((5-carboxypentyl)oxy)-5-(2-hydroxyacetamido)-4-(prop-2-yn-1-yloxy)tetrahydro-2H-pyran-2-carboxylic acid (5, 0.65 g, 1.29 mmol) in methanol (8.0 mL) and water (2.0 mL), was added zinc dust (0.84 g, 12.9 mmol) followed by acetic acid (0.37 mL, 6.45 mmol) drop-wise at 0° C. The reaction mixture was stirred at room temperature for 2 h then filtered. The filtrate was concentrated, and the crude residue obtained was purified by trituration with diethyl ether to afford the desired crude product (6). Yield: 0.45 g, 73.01%; LCMS m/z 477.10 [M+H]+.

### Synthesis of (2R,4S,5R,6R)-2-((5-carboxypentyl)oxy)-6-((1R,2R)-3-(2-(4-chlorophenyl)acetamido)-1,2-dihydroxypropyl)-5-(2-hydroxyacetamido)-4-(prop-2-yn-1-yloxy)tetrahydro-2H-pyran-2-carboxylic acid (Cpd. 20)

To a stirred solution of (2R,4S,5R,6R)-6-((1R,2R)-3-amino-1,2-dihydroxypropyl)-2-((5-carboxypentyl)oxy)-5-(2-hydroxyacetamido)-4-(prop-2-yn-1-yloxy)tetrahydro-2H-pyran-2-carboxylic acid (6, 0.25 g, 0.52 mmol) in N,N-dimethylformamide (3.0 mL), were successively added N,N-diisopropylethylamine (0.45 mL, 2.62 mmol) and perfluorophenyl 2-(4-chlorophenyl)acetate (7, 0.14 g, 0.42 mmol) at 0° C. The reaction mixture was stirred at room temperature for 4 h then concentrated under reduced pressure. The crude residue obtained was purified by prep HPLC using in water and acetonitrile as eluents to afford the desired product (Cpd. 20). Yield: 0.07 g, 21.21%; LCMS (ESI) m/z 629.35 [M+H]+. 1H NMR (400 MHz, Methanol-d4): δ 7.32-7.27 (m, 4H), 4.23 (d, J=2.4 Hz, 2H), 3.99 (s, 2H), 3.90-3.85 (m, 3H), 3.80-3.75 (m, 2H), 3.63 (dd, J=13.6 & 2.8 Hz, 1H), 3.53 (s, 2H), 3.43-3.41 (m, 1H), 3.34 (dd, J=8.8 & 1.2 Hz, 1H), 3.30-3.22 (m, 1H), 2.91 (dd, J=12.4 & 4.0 Hz, 1H), 2.84 (t, J=2.4 Hz, 1H), 2.28 (t, J=7.2 Hz, 2H), 1.66-1.55 (m, 5H), 1.41-1.36 (m, 2H).

### Compound 21

### (2R,4S,5R,6R)-2-(benzyloxy)-6-((1R,2R)-3-(2-(4-chlorophenyl)acetamido)-1,2-dihydroxypropyl)-4-hydroxy-5-(3-(2-(2-(prop-2-yn-1-yloxy)ethoxy)ethoxy)propanamido)tetrahydro-2H-pyran-2-carboxylic acid (Cpd. 21)

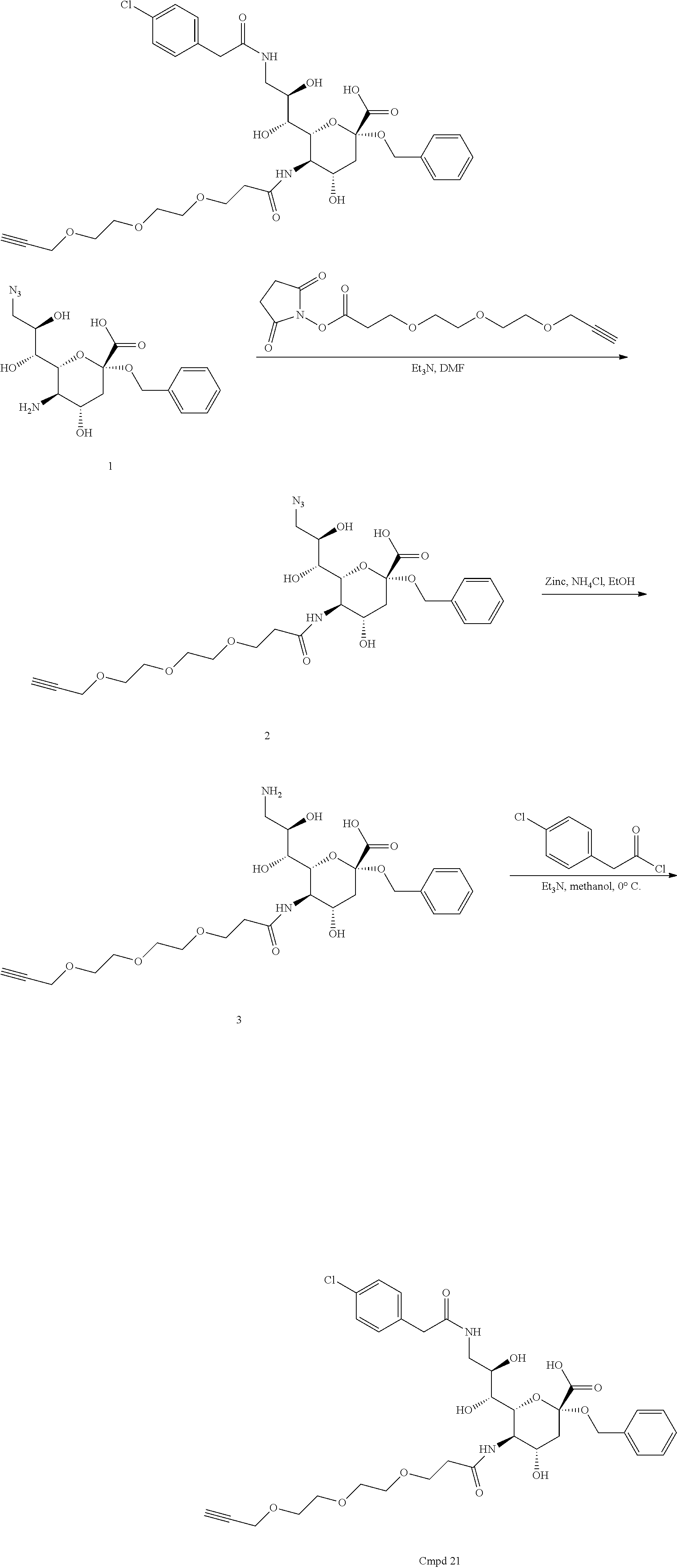

To a solution of Amine (1) (72.0 mg, 188 umol) and triethylamine (78.7 uL, 565 umol) in DMF (4.71 mL) at 0° C. was added Propargyl-PEG3-NHSester (245 uL, 245 umol) drop-wise. The cooling bath was removed, and the mixture was stirred overnight at room temperature. Water was added, followed by 1N HCl and the mixture was concentrated. The crude residue obtained was purified by silica-gel chromatography using ethyl acetate and methanol as eluents to afford the desired product (2). Yield: 90.0 mg, 82%; LCMS (ESI) m/z 603 [M+Na]+.

To a stirred solution of (2) (41.0 mg, 70.6 umol) in EtOH (2.14 mL) at room temperature was added Zinc (462 mg, 7.06 mmol), followed by an aqueous solution of Ammonium chloride (706 uL, 706 umol) dropwise. The reaction mixture was stirred at room temperature for 10 min, filtered then concentrated. The crude residue obtained was purified by reverse-phase column chromatography using water and acetonitrile as eluents (+0.1% TFA) to afford the desired product (3). Yield: 39.2 mg; 46%. LCMS (ESI) m/z 555 [M+H]+.

Amine (3) (40.0 mg, 93.8 umol) was dissolved in MeOH (782 uL) and the mixture was cooled down to 0° C. After 5 min of stirring, N,N-Diisopropylethylamine (163 uL, 938 umol) was added followed 5 min later by 4-chlorophenylacetyl chloride (13.7 uL, 93.8 umol). The mixture was stirred at 0° C. for 2 h, overnight at room temperature then diluted with water and concentrated. The crude residue obtained was purified by silica-gel chromatography using ethyl acetate and methanol as eluents to afford the desired compound (Cpd. 21). Yield: 54.3 mg; 54%. LCMS (ESI) m/z 707 [M+H]+.

### Compound 22

### (2R,4S,5R,6R)-5-(3-(3,6,9,12,15-pentaoxaoctadec-17-yn-1-yl)ureido)-2-(benzyloxy)-6-((1R,2R)-3-(2-(4-chlorophenyl)acetamido)-1,2-dihydroxypropyl)-4-hydroxytetrahydro-2H-pyran-2-carboxylic acid (Cpd. 22)

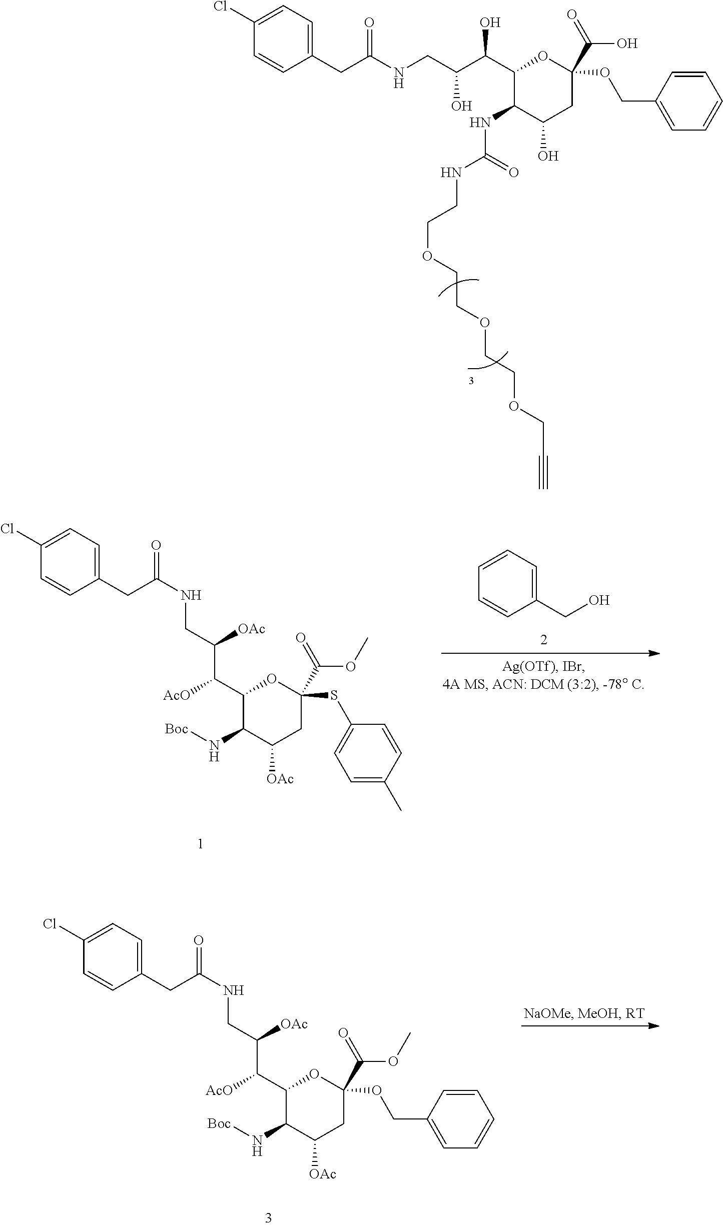

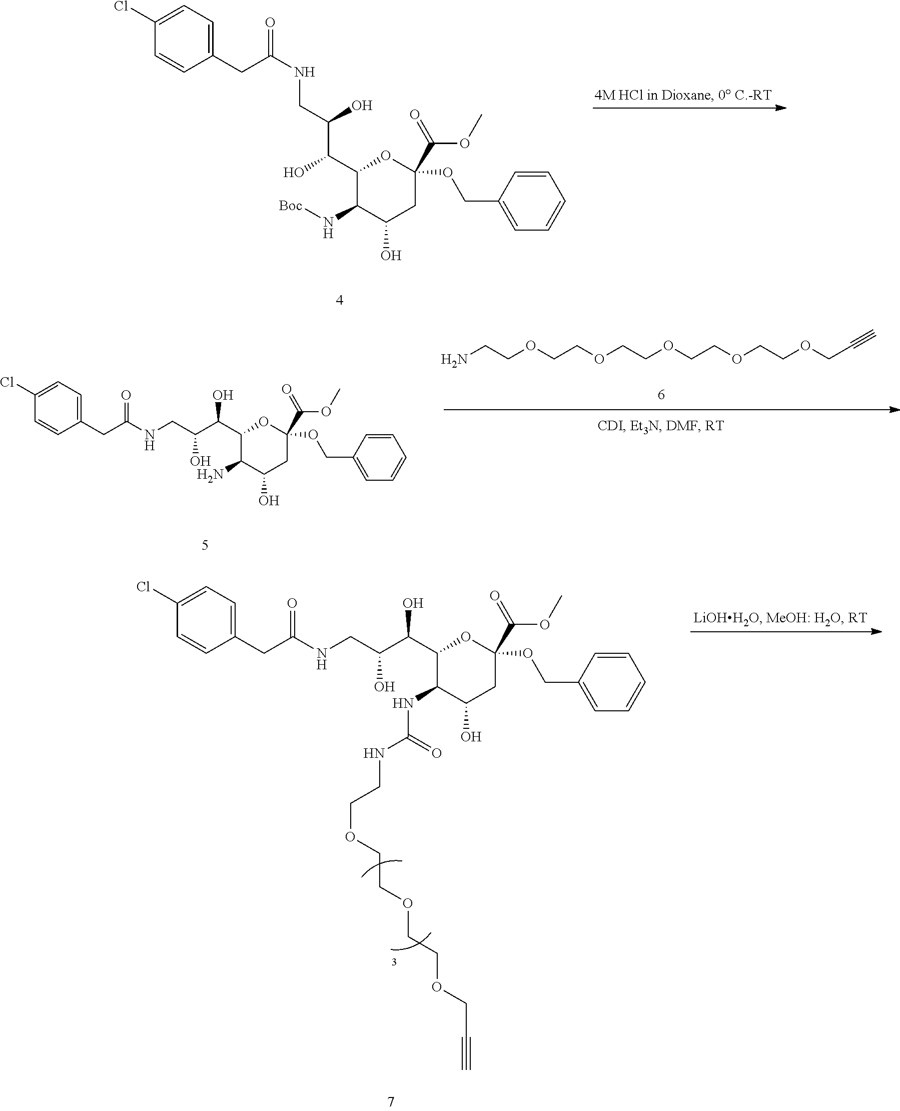

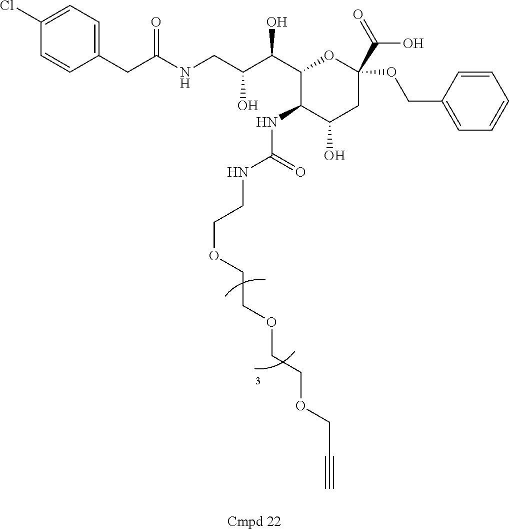

### Synthesis of (1R,2R)-1-((2R,3R,4S,6R)-4-acetoxy-6-(benzyloxy)-3-((tert-butoxycarbonyl)amino)-6-(methoxycarbonyl)tetrahydro-2H-pyran-2-yl)-3-(2-(4-chlorophenyl)acetamido)propane-1,2-diyl diacetate (3)

A mixture of (1R,2R)-1-((2R,3R,4S,6R)-4-acetoxy-3-((tert-butoxycarbonyl)amino)-6-(methoxycarbonyl)-6-(p-tolylthio)tetrahydro-2H-pyran-2-yl)-3-(2-(4-chlorophenyl)acetamido)propane-1,2-diyl diacetate (1, 0.25 g, 0.33 mmol), phenylmethanol (2, 0.11 g, 0.98 mmol), silver(1+) trifluoromethanesulfonate (0.21 g, 0.82 mmol) and activated 4 Å powdered molecular sieves (0.1 g) in anhydrous dichloromethane (15.0 mL) and anhydrous acetonitrile (30.0 mL) was stirred at room temperature for 1 h. The solution was cooled to −78° C. and a solution of iodobromane (0.10 g, 0.49 mmol) in dichloromethane (5.0 mL) was added dropwise. The reaction mixture was stirred at same temperature for 2 h then quenched by carefull addition of triethylamine (0.2 mL) and warmed to room temperature. The mixture was filtered, the filtrate was washed by a saturated solution of sodium bicarbonate then dried, filtered and concentrated. The crude residue obtained was purified by silica-gel column chromatography using ethyl acetate and heptane as eluents to afford the desired product (3). Yield: 0.12 g, 49.03%; LCMS m/z 748.70 [M+H]+.

### Synthesis of methyl (2R,4S,5R,6R)-2-(benzyloxy)-5-((tert-butoxycarbonyl)amino)-6-((1R,2R)-3-(2-(4-chlorophenyl)acetamido)-1,2-dihydroxypropyl)-4-hydroxytetrahydro-2H-pyran-2-carboxylate (4)

To a stirred solution of (1R,2R)-1-((2R,3R,4S,6R)-4-acetoxy-6-(benzyloxy)-3-((tert-butoxycarbonyl)amino)-6-(methoxycarbonyl)tetrahydro-2H-pyran-2-yl)-3-(2-(4-chlorophenyl)acetamido)propane-1,2-diyl diacetate (3, 0.20 g, 0.27 mmol) in methanol (10.0 mL) was added sodium methoxide (0.014 g, 0.27 mmol) portion wise at 0° C. until pH˜9-10. The reaction mixture was stirred at room temperature for 4 h then was acidified with acidic (Dowex 50, H⁺) to pH˜6. The suspension was filtered, and the filtrate was concentrated. The crude residue obtained was triturated with diethylether to afford the desired crude product (4). Yield: 0.16 g, crude; LCMS m/z 622.75 [M+H]⁺.

### Synthesis of methyl (2R,4S,5R,6R)-5-amino-2-(benzyloxy)-6-((1R,2R)-3-(2-(4-chlorophenyl)acetamido)-1,2-dihydroxypropyl)-4-hydroxytetrahydro-2H-pyran-2-carboxylate (5)

To a stirred solution of methyl (2R,4S,5R,6R)-2-(benzyloxy)-5-((tert-butoxycarbonyl)amino)-6-((1R,2R)-3-(2-(4-chlorophenyl)acetamido)-1,2-dihydroxypropyl)-4-hydroxytetrahydro-2H-pyran-2-carboxylate (4, 0.15 g, 0.27 mmol), in 1,4 dioxane (4.0 mL), 4.0 M hydrochloric acid in 1,4 dioxane (2.0 mL) was added at 0° C. and the reaction mixture was stirred at room temperature for 3 h. The solvent was concentrated to afford the desired crude product (5) which was used without further purification. Yield: 0.10 g, Crude; LCMS (ESI) m/z 522.85 [M+H]⁺.

### Synthesis of methyl (2R,4S,5R,6R)-5-(3-(3,6,9,12,15-pentaoxaoctadec-17-yn-1-yl)ureido)-2-(benzyloxy)-6-((1R,2R)-3-(2-(4-chlorophenyl)acetamido)-1,2-dihydroxypropyl)-4-hydroxytetrahydro-2H-pyran-2-carboxylate (6)

To a stirred solution of methyl (2R,4S,5R,6R)-5-amino-2-(benzyloxy)-6-((1R,2R)-3-(2-(4-chlorophenyl)acetamido)-1,2-dihydroxypropyl)-4-hydroxytetrahydro-2H-pyran-2-carboxylate (5, 0.11 g, 0.21 mmol) in anhydrous dimethylformamide (2.0 mL) was added 1,1′-Carbonyldiimidazole (0.034 g, 0.21 mmol) and triethylamine (0.09 mL, 0.63 mmol) at 0° C. followed by addition of 3,6,9,12,15-pentaoxaoctadec-17-yn-1-amine (0.12 g, 0.42 mmol). The reaction mixture was stirred at room temperature for 12 then concentrated. The crude residue obtained was triturated with diethyl ether to afford the desired crude product (6). Yield: 0.10 g, crude; LCMS m/z 824.20 [M+H]⁺.

### Synthesis of (2R,4S,5R,6R)-5-(3-(3,6,9,12,15-pentaoxaoctadec-17-yn-1-yl)ureido)-2-(benzyloxy)-6-((1R,2R)-3-(2-(4-chlorophenyl)acetamido)-1,2-dihydroxypropyl)-4-hydroxytetrahydro-2H-pyran-2-carboxylic acid (Cpd. 22)

To a stirred solution of methyl (2R,4S,5R,6R)-5-(3-(3,6,9,12,15-pentaoxaoctadec-17-yn-1-yl)ureido)-2-(benzyloxy)-6-((1R,2R)-3-(2-(4-chlorophenyl)acetamido)-1,2-dihydroxypropyl)-4-hydroxytetrahydro-2H-pyran-2-carboxylate (6, 0.13 g, 0.43 mmol) in methanol (5 mL), was added a solution of lithium hydroxide monohydrate (0.007 g, 0.86 mmol) in water (0.5 mL). The reaction mixture was stirred at room temperature for 6 then acidified with acidic (Dowex 50, H⁺) to pH˜6. The suspension was filtered, and the filtrate was concentrated. The crude residue obtained was purified by prep HPLC using water and acetonitrile (+0.1% TFA) to afford the desired product (Cpd. 22). Yield: 0.041 g, 32.08%; LCMS (ESI) m/z 810.40 [M+H]⁺. ¹H NMR (400 MHz, Methanol-d₄): δ 7.32-7.25 (m, 9H), 4.80 (d, J=11.2 Hz, 1H), 4.48 (d, J=11.2 Hz, 1H), 4.18 (d, J=2.4 Hz, 2H), 3.93 (td, J=8.4 Hz & 3.2 Hz, 1H), 3.69-3.48 (m, 27H), 3.32-3.31 (m, 1H), 2.85 (t, J=2.4 Hz, 1H), 2.78 (dd, J=12.4 Hz & 4.0 Hz, 1H), 1.78 (t, J=12.0 Hz, 1H).

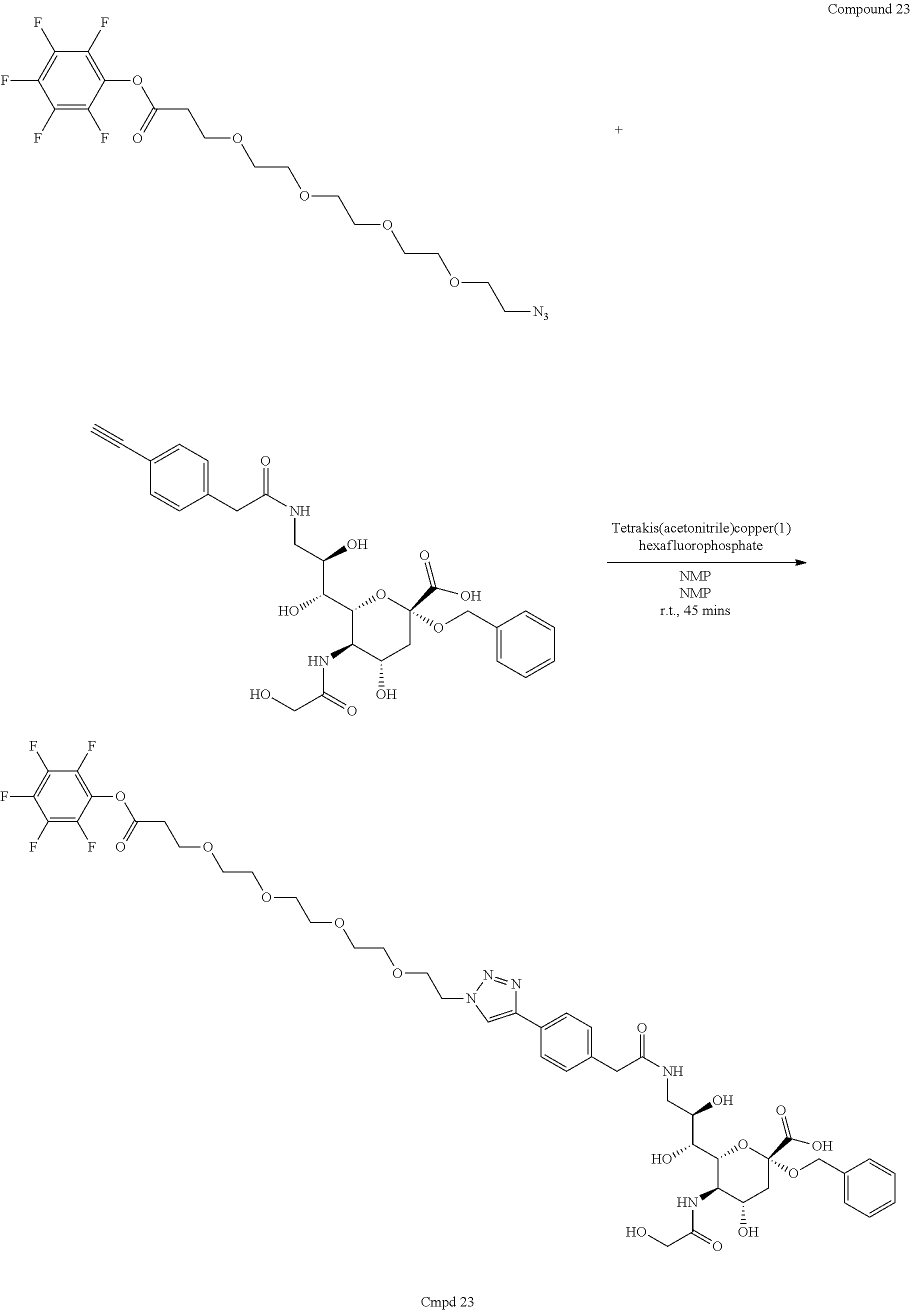 [SMILES: [N-]=[N+]=NCCOCCOCCOCCOCCC(=O)Oc1c(F)c(F)c(F)c(F)c1F ; C#Cc1ccc(CC(=O)NC[C@@H](O)[C@@H](O)[C@@H]2O[C@@](OCc3ccccc3)(C(=O)O)C[C@H](O)[C@H]2NC(=O)CO)cc1 ; O=C(Cc1ccc(-c2cn(CCOCCOCCOCCOCCC(=O)Oc3c(F)c(F)c(F)c(F)c3F)nn2)cc1)NC[C@@H](O)[C@@H](O)[C@@H]1O[C@@](OCc2ccccc2)(C(=O)O)C[C@H](O)[C@H]1NC(=O)CO]

To a solution of alkyne (27.4 mg, 49.2 umol) in anhydrous NMP (324 uL) was added Azido-PEG4-PFP-ester (25.2 mg, 55.1 umol) in anhydrous NMP (324 uL) dropwise, followed by Tetrakis(acetonitrile)copper(l) hexafluorophosphate (45.9 mg, 123 umol). The mixture was stirred for 30 mins at room temperature, filtered through a 0.2 μm PTFE filter and the filtrate was purified by preparatory HPLC using acetonitrile and water (+0.1% TFA) as eluents to afford the desired product (Cpd. 23). Yield: 29 mg; 58%. LCMS (ESI) m/z 1014 [M+H]+.

### Compound 24

### (2R,4S,5R,6R)-6-((1R,2R)-3-(2-(4-ethynylphenyl)acetamido)-1,2-dihydroxypropyl)-2-(hexyloxy)-4-hydroxy-5-(2-hydroxyacetamido)tetrahydro-2H-pyran-2-carboxylic acid (Cpd. 24)

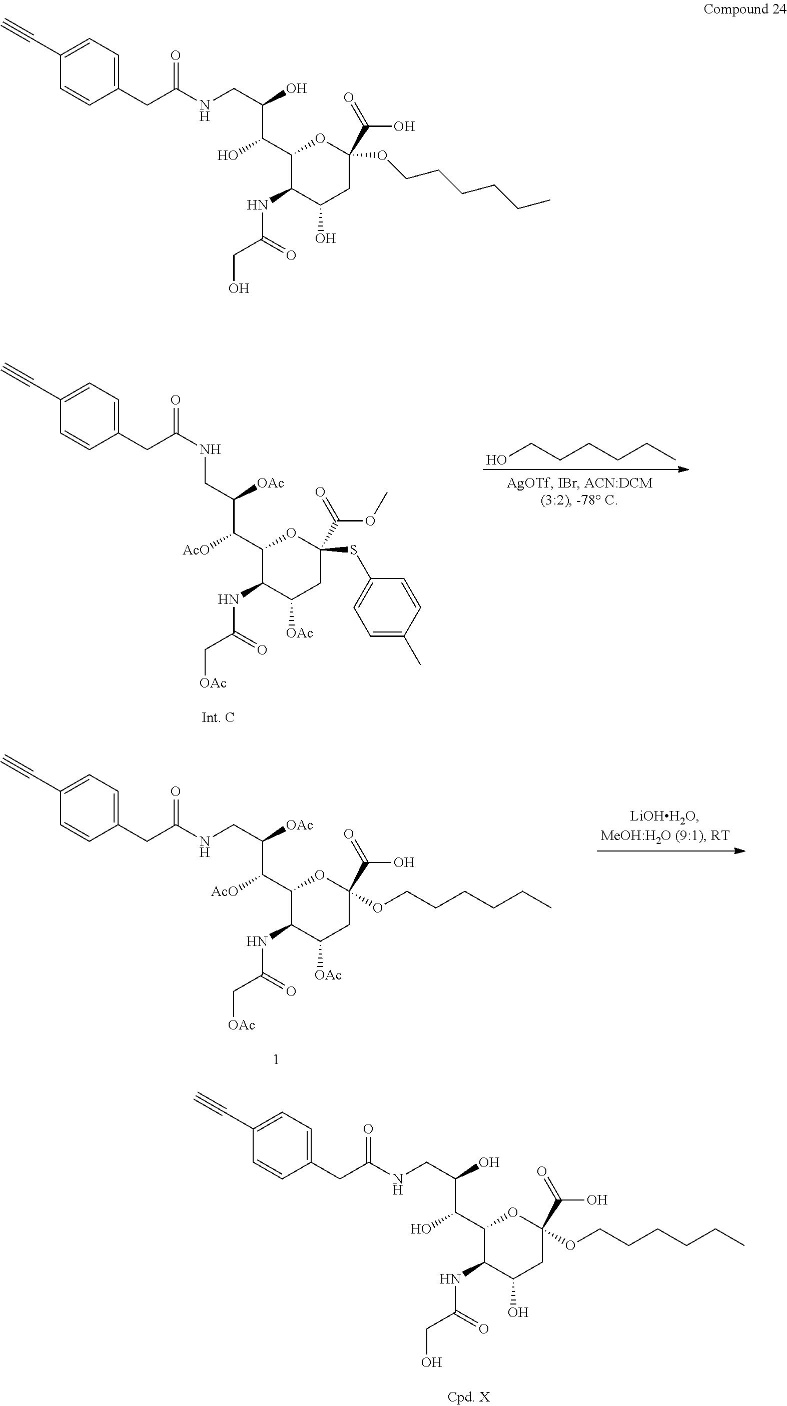

### Synthesis of (1R,2R)-1-((2R,3R, 4S, 6R)-4-acetoxy-3-(2-acetoxyacetamido)-6-(hexyloxy)-6-(methoxycarbonyl)tetrahydro-2H-pyran-2-yl)-3-(2-(4-ethynylphenyl)acetamido)propane-1,2-diyl diacetate (1)

To a stirred solution of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-ethynylphenyl)acetamido) propane-1,2-diyl diacetate (Int. C, 0.064 g, 84.8 umol) and hexan-1-ol (26.6 uL, 212 umol) in anhydrous dichloromethane (1.13 mL) and anhydrous acetonitrile (1.70 mL), silver(1+) trifluoromethanesulfonate (0.044 g, 170 umol)) and activated 4 Å powdered molecular sieves (250 mg) were added, and the solution was stirred in the dark at room temperature for 1 h under nitrogen atmosphere. The solution was cooled to −78° C., and a solution of iodine monobromide (26.3 mg, 127 umol) in dichloromethane (0.127 mL) was added dropwise. The mixture was stirred at the same temperature for 3 h. The reaction mixture was quenched with N,N-diisopropylethylamine (100 uL) and allowed to warm to room temperature. The reaction mixture was filtered, and the filtrate was washed with an aq. sat. solution of sodium bicarbonate. The organic phase was dried, filtered, and concentrated. The crude residue obtained was purified by column chromatography using ethyl acetate and hexane as eluents to afford the desired product (1). Yield: 0.025 g, 40%; LCMS m/z 731.40 [M−H]⁻.

### Synthesis of (2R,4S,5R,6R)-6-((1R,2R)-3-(2-(4-ethynylphenyl)acetamido)-1,2-dihydroxypropyl)-2-(hexyloxy)-4-hydroxy-5-(2-hydroxyacetamido)tetrahydro-2H-pyran-2-carboxylic acid (Cpd. 24)

(1R,2R)-1-((2R,3R,4S,6R)-4-acetoxy-3-(2-acetoxyacetamido)-6-(hexyloxy)-6-(methoxycarbonyl)tetrahydro-2H-pyran-2-yl)-3-(2-(4-ethynylphenyl)acetamido)propane-1,2-diyl diacetate (1, 0.020 g, 0.033 mmol) in methanol (0.198 ml) was added a solution of lithium hydroxide monohydrate (2.37 mg, 0.099 mmol) in water (0.022 ml). The reaction mixture was stirred at room temperature for 6 h. After completion, the reaction mixture was treated with acidic resin (Dowex 50, H+) to pH˜6 and the suspension was filtered. The filtrate was concentrated under reduced pressure and the crude residue obtained was purified by prep HPLC using acetonitrile and water (+0.1% TFA) as eluents to afford the desired product (Cpd. 24). Yield: 11.2 mg, 62%; LCMS (ESI) m/z 549.30 [M−H]⁻. ¹H NMR (500 MHz, DMSO-d₆): 7.98 (t, J=5.5 Hz, 1H), 7.83 (d, J=7.5 Hz, 1H), 7.38 (d, J=8.0 Hz, 2H), 7.26 (d, J=8.0 Hz, 2H), 5.53 (t, J=5.5 Hz, 1H), 4.94 (d, J=6.0 Hz, 1H), 4.70 (d, J=5.0 Hz, 1H), 4.11 (s, 1H), 3.87 (m, 2H), 3.79 (m br, 1H), 3.67-3.59 (m, 2H), 3.57-3.51 (m, 3H), 3.46 (m, 2H), 3.28-3.26 (m, 2H), 3.21-3.18 (m, 2H), 2.94-2.89 (m, 1H), 1.49 (dd, J=12.0 Hz, 1H), 1.46-1.42 (m, 2H), 1.28-1.23 (m, 6H), 0.85 (t, J=7.0 Hz, 3H).

### Compound 25

### (2R,4S,5R,6R)-6-((1R,2R)-3-(2-(4-ethynylphenyl)acetamido)-1,2-dihydroxypropyl)-4-hydroxy-5-(2-hydroxyacetamido)-2-(2-(2-(2-methoxyethoxy)ethoxy)ethoxy)tetrahydro-2H-pyran-2-carboxylic acid (Cpd. 25)

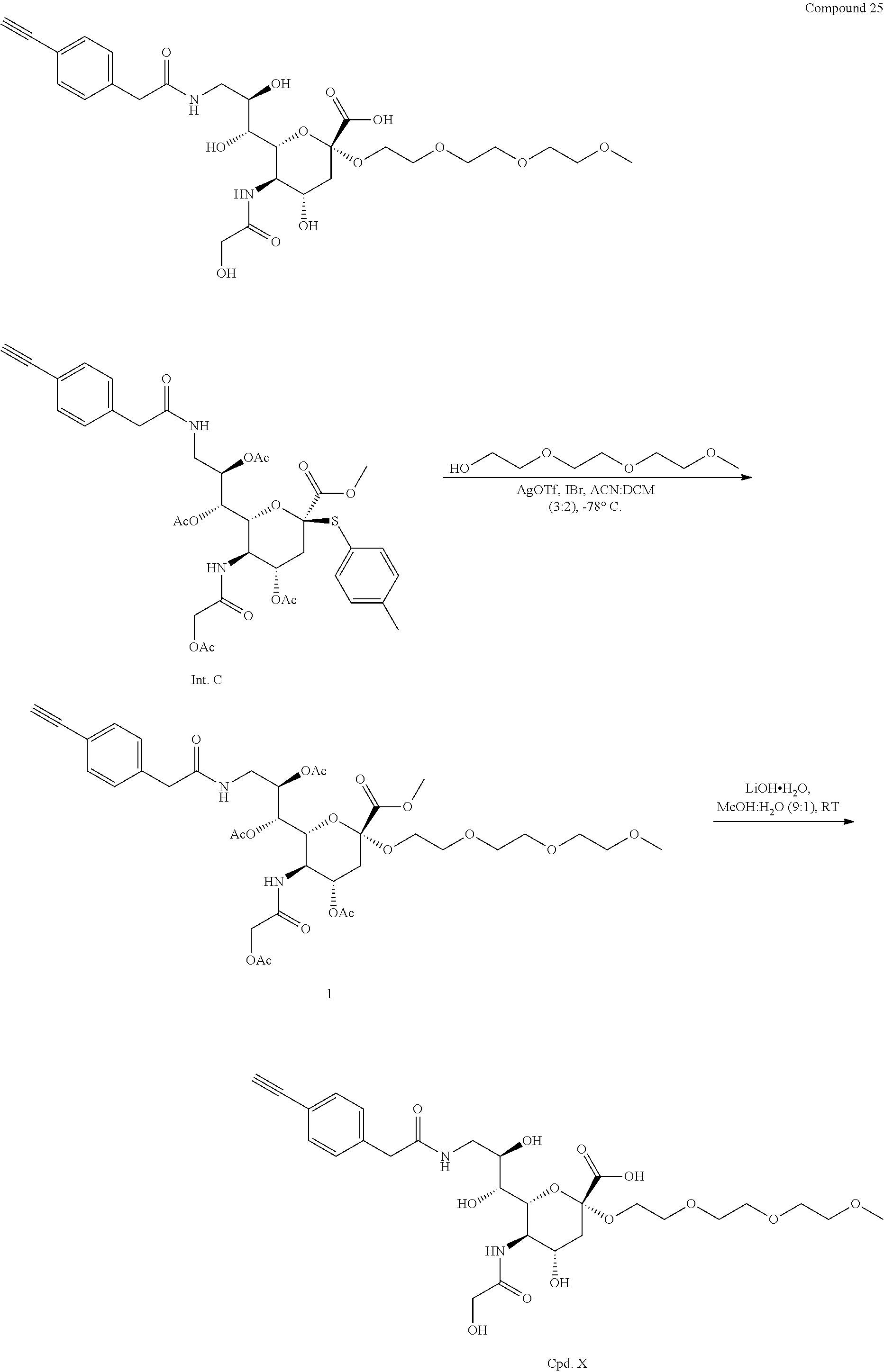

### Synthesis of (1R,2R)-1-((2R,3R,4S,6R)-4-acetoxy-3-(2-acetoxyacetamido)-6-(methoxycarbonyl)-6-(2-(2-(2-methoxyethoxy)ethoxy)ethoxy)tetrahydro-2H-pyran-2-yl)-3-(2-(4-ethynylphenyl) acetamido)propane-1,2-diyl diacetate (1)

To a stirred solution of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-ethynylphenyl)acetamido) propane-1,2-diyl diacetate (Int. C, 0.147 g, 195 umol) and 2-(2-(2-methoxyethoxy)ethoxy)ethan-1-ol (77.9 uL, 487 umol) in anhydrous dichloromethane (2.60 mL) and anhydrous acetonitrile (3.90 mL), silver(1+) trifluoromethanesulfonate (0.100 g, 390 umol)) and activated 4 Å powdered molecular sieves (500 mg) were added, and the solution was stirred in the dark at room temperature for 1 h under nitrogen atmosphere. The solution was cooled to −78° C., and a solution of iodine monobromide (60.4 mg, 292 umol) in dichloromethane (0.292 mL) was added dropwise. The mixture was stirred at the same temperature for 3 h. The reaction mixture was quenched with N,N-diisopropylethylamine (200 uL) and allowed to warm to room temperature. The reaction mixture was filtered, and the filtrate was washed with an aq. sat. solution of sodium bicarbonate. The organic phase was dried, filtered, and concentrated. The crude residue obtained was purified by column chromatography using ethyl acetate and hexane as eluents to afford the desired product (1). Yield: 0.102 g, 66%; LCMS m/z 793.40 [M−H]⁻.

### Synthesis of (2R,4S,5R,6R)-6-((1R,2R)-3-(2-(4-ethynylphenyl)acetamido)-1,2-dihydroxypropyl)-4-hydroxy-5-(2-hydroxyacetamido)-2-(2-(2-(2-methoxyethoxy)ethoxy)ethoxy)tetrahydro-2H-pyran-2-carboxylic acid (Cpd. 25)

(1R,2R)-1-((2R,3R,4S,6R)-4-acetoxy-3-(2-acetoxyacetamido)-6-(methoxycarbonyl)-6-(2-(2-(2-methoxyethoxy)ethoxy)ethoxy)tetrahydro-2H-pyran-2-yl)-3-(2-(4-ethynylphenyl)acetamido) propane-1,2-diyl diacetate (1, 0.102 g, 0.128 mmol) in methanol (0.77 ml) was added a solution of lithium hydroxide monohydrate (0.0184 g, 0.770 mmol) in water (0.085 ml). The reaction mixture was stirred at room temperature for 6 h. After completion, the reaction mixture was treated with acidic resin (Dowex 50, H+) to pH˜6 and the suspension was filtered. The filtrate was concentrated under reduced pressure and the crude residue obtained was purified by prep HPLC using acetonitrile and water (+0.1% TFA) as eluents to afford the desired product (Cpd. 25). Yield: 58.0 mg, 74%; LCMS (ESI) m/z 611.5 [M−H]⁻. ¹H NMR (500 MHz, DMSO-d₆): 7.99 (t, J=5.5 Hz, 1H), 7.84 (d, J=7.5 Hz, 1H), 7.39 (d, J=8.0 Hz, 2H), 7.26 (d, J=8.0 Hz, 2H), 5.54 (br, 1H), 4.94 (br, 1H), 4.70 (br, 1H), 4.27 (br, 1H), 4.12 (s, 1H), 3.88-3.85 (m, 2H), 3.83-3.79 (br, 1H), 3.79-3.72 (m, 1H), 3.68-3.64 (m, 1H), 3.57-3.54 (m, 2H), 3.51-3.49 (m, 9H), 3.47-3.45 (m, 3H), 3.44-3.42 (m, 3H), 3.23 (s, 3H), 3.21-3.19 (m, 1H), 2.97-2.92 (m, 1H), 1.53 (dd, J=12.0 Hz, 1H).

### Compound 26

### (2R,4S,5R,6R)-6-((1R,2R)-3-(2-(4-ethynylphenyl)acetamido)-1,2-dihydroxypropyl)-4-hydroxy-5-(2-hydroxyacetamido)-2-((6-((2-((2-methoxyethyl)amino)-3,4-dioxocyclobut-1-en-1-yl)amino)hexyl)oxy)tetrahydro-2H-pyran-2-carboxylic acid (Cpd. 26)

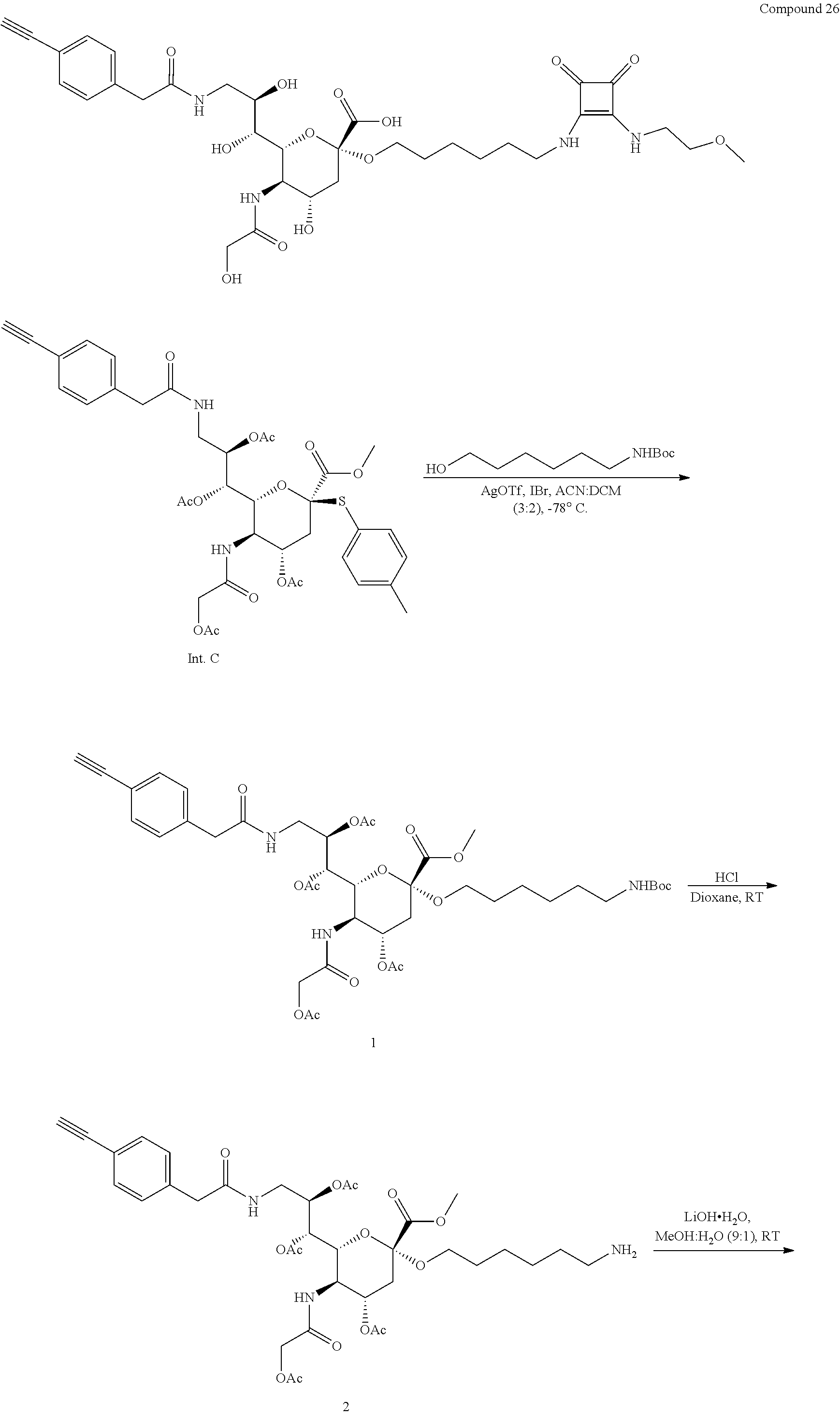

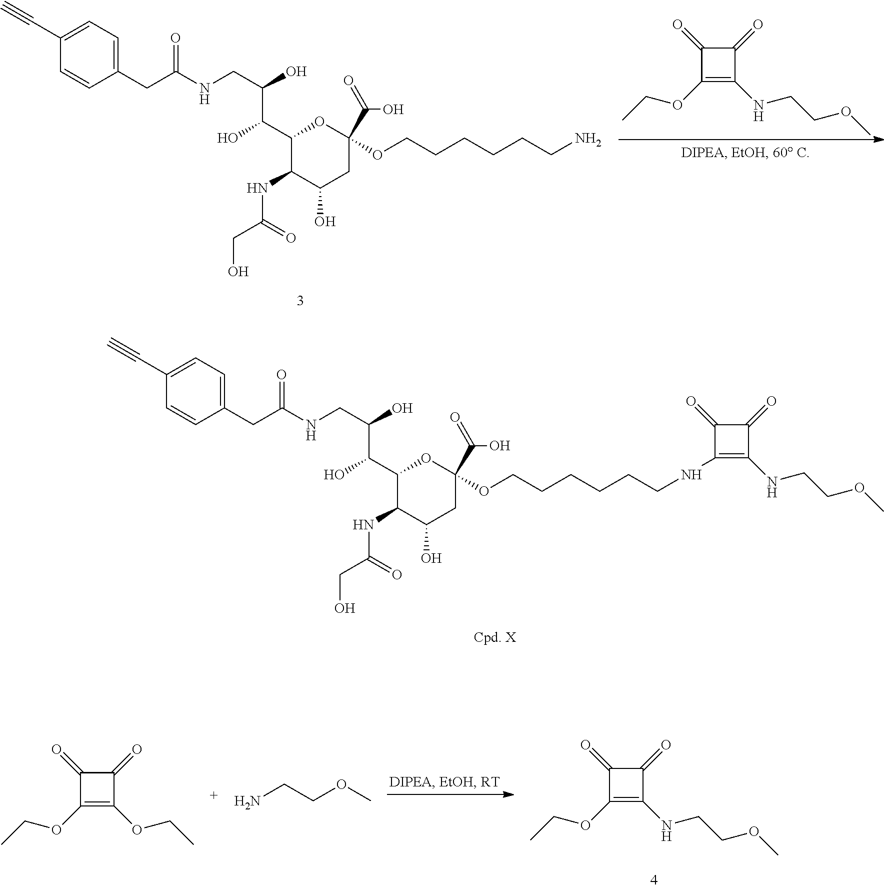

### Synthesis of (1R,2R)-1-((2R,3R,4S,6R)-4-acetoxy-3-(2-acetoxyacetamido)-6-((6-((tert-butoxy carbonyl)amino)hexyl)oxy)-6-(methoxycarbonyl)tetrahydro-2H-pyran-2-yl)-3-(2-(4-ethynylphenyl)acetamido)propane-1,2-diyl diacetate (1)

To a stirred solution of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-ethynylphenyl)acetamido) propane-1,2-diyl diacetate (Int. C, 0.200 g, 268 umol) and tert-butyl (6-hydroxyhexyl)carbamate (0.087 g, 401 umol) in anhydrous dichloromethane (3.57 mL) and anhydrous acetonitrile (5.35 mL), silver(1+) trifluoromethanesulfonate (0.172 g, 669 umol)) and activated 4 Å powdered molecular sieves (600 mg) were added, and the solution was stirred in the dark at room temperature for 1 h under nitrogen atmosphere. The solution was cooled to −78° C., and a solution of iodine monobromide (83.0 mg, 401 umol) in dichloromethane (0.401 mL) was added dropwise. The mixture was stirred at the same temperature for 3 h. The reaction mixture was quenched with N,N-diisopropylethylamine (280 uL) and allowed to warm to room temperature. The reaction mixture was filtered, and the filtrate was washed with an aq. sat. solution of sodium bicarbonate. The organic phase was dried, filtered, and concentrated. The crude residue obtained was purified by column chromatography using ethyl acetate and hexane as eluents to afford the desired product (1). Yield: 0.157 g, 69%; LCMS m/z 846.40 [M−H]⁻.

### Synthesis of (1R,2R)-1-((2R,3R,4S,6R)-4-acetoxy-3-(2-acetoxyacetamido)-6-((6-aminohexyl)oxy)-6-(methoxycarbonyl)tetrahydro-2H-pyran-2-yl)-3-(2-(4-ethynylphenyl)acetamido)propane-1,2-diyl diacetate (2)

To a stirred solution of (1R,2R)-1-((2R,3R,4S,6R)-4-acetoxy-3-(2-acetoxyacetamido)-6-((6-((tert-butoxycarbonyl)amino)hexyl)oxy)-6-(methoxycarbonyl)tetrahydro-2H-pyran-2-yl)-3-(2-(4-ethynylphenyl)acetamido)propane-1,2-diyl diacetate (157 mg, 185 umol) in anhydrous dioxane (2.89 mL), hydrochloric acid (4M in dioxane, 1.16 mL) was added dropwise at 0° C. The mixture was stirred overnight at room temperature and then concentrated to afford the desired crude product (2), which was used in the next step without further purification.

### Synthesis of (2R,4S,5R,6R)-2-((6-aminohexyl)oxy)-6-((1R,2R)-3-(2-(4-ethynylphenyl)acetamido)-1,2-dihydroxypropyl)-4-hydroxy-5-(2-hydroxyacetamido)tetrahydro-2H-pyran-2-carboxylic acid (3)

(1R,2R)-1-((2R,3R,4S,6R)-4-acetoxy-3-(2-acetoxyacetamido)-6-((6-aminohexyl)oxy)-6-(methoxycarbonyl)tetrahydro-2H-pyran-2-yl)-3-(2-(4-ethynylphenyl)acetamido)propane-1,2-diyl diacetate (2, 0.138 g, 0.185 mmol) in methanol (1.11 ml) was added a solution of lithium hydroxide monohydrate (26.5 mg, 1.11 mmol) in water (0.123 ml). The reaction mixture was stirred at room temperature for 6 h. After completion, the reaction mixture was treated with acidic resin (Dowex 50, H+) to pH˜6 and the suspension was filtered. The filtrate was concentrated under reduced pressure and the crude residue obtained was purified by prep HPLC using acetonitrile and water (+0.1% TFA) as eluents to afford the desired product (3). Yield: 22.3 mg, 21% over 2 steps; LCMS (ESI) m/z 566.30 [M+H]⁺.

### Synthesis of 3-ethoxy-4-((2-methoxyethyl)amino)cyclobut-3-ene-1,2-dione (4)

To a stirred solution of 3,4-diethoxycyclobut-3-ene-1,2-dione (513 mg, 3.01 mmol) in EtOH (5.11 mL), 2-methoxyethan-1-amine (226 mg, 3.01 mmol) was added at 0° C., followed by N,N-diisopropylethylamine (1.58 mL, 9.04 mmol). The mixture was stirred for 2 h at room temperature and then concentrated to afford the desired product (4) which was used in the next step without further purification. Yield: 431 mg, 72%.

### Synthesis of (2R,4S,5R,6R)-6-((1R,2R)-3-(2-(4-ethynylphenyl)acetamido)-1,2-dihydroxypropyl)-4-hydroxy-5-(2-hydroxyacetamido)-2-((6-((2-((2-methoxyethyl)amino)-3,4-dioxocyclobut-1-en-1-yl)amino)hexyl)oxy)tetrahydro-2H-pyran-2-carboxylic acid (Cpd. 26)

To a stirred solution of (2R,4S,5R,6R)-2-((6-aminohexyl)oxy)-6-((1R,2R)-3-(2-(4-ethynylphenyl) acetamido)-1,2-dihydroxypropyl)-4-hydroxy-5-(2-hydroxyacetamido)tetrahydro-2H-pyran-2-carboxylic acid (22.0 mg, 38.9 umol) in EtOH (389 uL), N,N-diisopropylethylamine (20.3 uL, 117 umol) was added at 0° C., followed by 3-ethoxy-4-((2-methoxyethyl)amino)cyclobut-3-ene-1,2-dione (7.75 mg, 38.9 umol). The mixture was stirred for 6 h at 60° C. and then filtered at room temperature. The filtrate was concentrated under reduced pressure and the crude residue obtained was purified by prep HPLC using acetonitrile and water (+0.1% TFA) as eluents to afford the desired product (Cpd. 26). Yield: 14.2 mg, 51%; LCMS (ESI) m/z 719.30 [M+H]⁺. ¹H NMR (500 MHz, DMSO-d₆): 7.98 (br, 1H), 7.80 (br, 1H), 7.38 (d, J=8.5 Hz, 2H), 7.26 (d, J=8.5 Hz, 2H), 5.52 (t, J=5.5 Hz, 1H), 4.70 (d, J=5.0 Hz, 1H), 4.11 (s, 1H), 3.87 (m, 2H), 3.78-3.73 (m, 1H), 3.68-3.62 (m, 3H), 3.59-3.45 (m, 10H), 3.27 (s, 3H), 3.19-3.13 (m, 1H), 3.02-2.95 (m, 1H), 2.92-2.84 (m, 1H), 1.53-1.39 (m, 5H), 1.33-1.20 (m, 4H).

### Compound 27

### (2R,4S,5R,6R)-6-((1R,2R)-3-(2-(4-ethynylphenyl)acetamido)-1,2-dihydroxypropyl)-4-hydroxy-5-(2-hydroxyacetamido)-2-((6-(1-(2-(2-methoxyethoxy)ethyl)-1H-1,2,3-triazol-4-yl)hexyl)oxy)tetrahydro-2H-pyran-2-carboxylic acid (Cpd. 27)

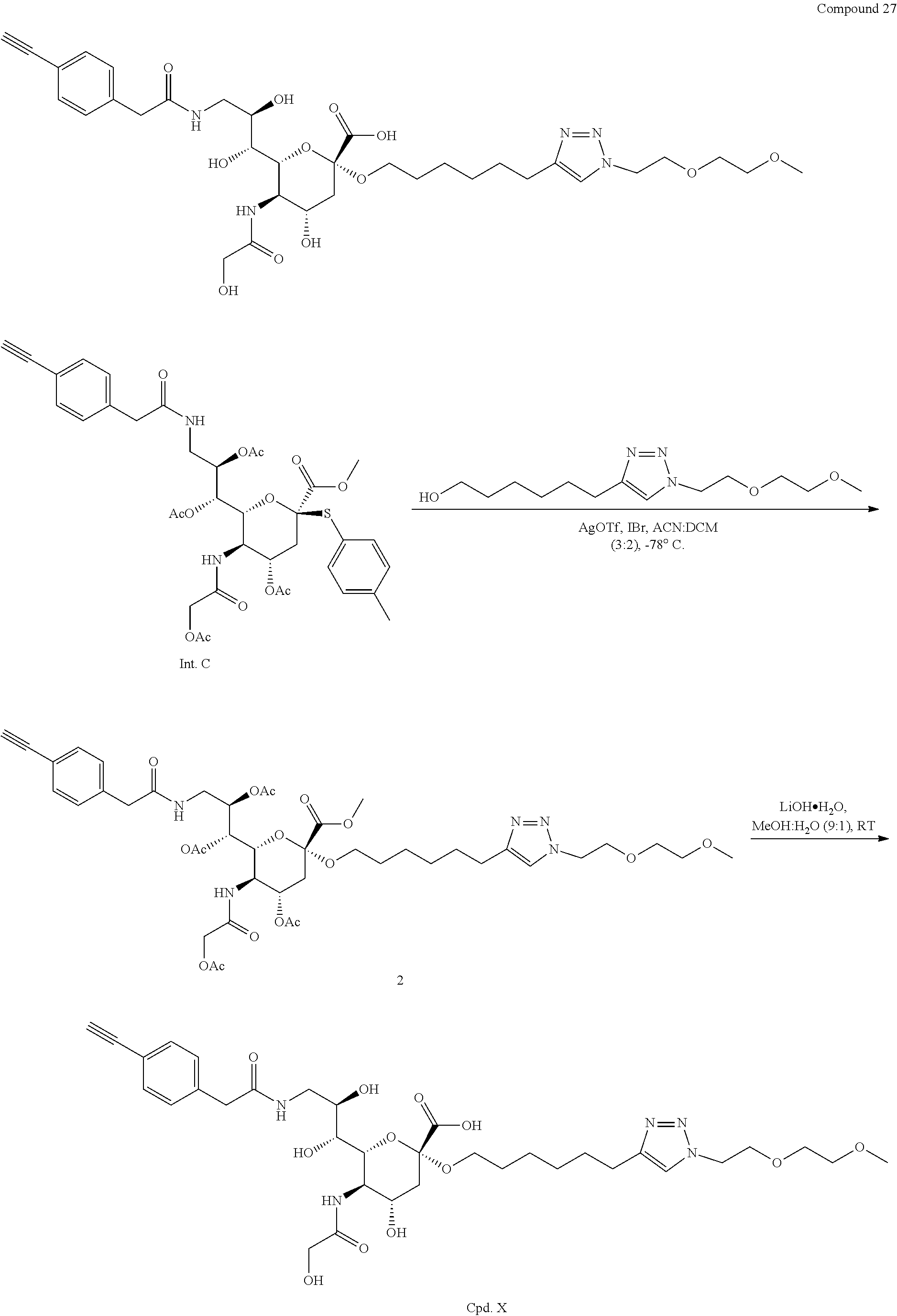

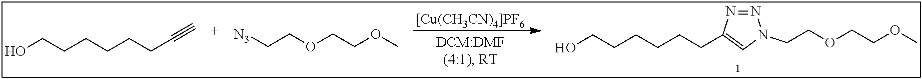

### Synthesis of 6-(1-(2-(2-methoxyethoxy)ethyl)-1H-1,2,3-triazol-4-yl)hexan-1-ol (1)

To a solution of oct-7-yn-1-ol (150 mg, 1.19 mmol) in anhydrous DCM (4.75 mL) and DMF (1.19 mL), 1-azido-2-(2-methoxyethoxy)ethane (173 mg, 1.19 mmol) was added followed by tetrakis(acetonitrile)copper(l) hexafluorophosphate (0.886 g, 2.38 mmol). The mixture was stirred for 45 min at room temperature, then diluted with DCM (50 mL) and washed with an aq. solution of Na₄EDTA (0.1 M, 3×50 mL). The organic phase was dried, filtered, and concentrated. The crude residue obtained was purified by column chromatography using ethyl acetate as eluent to afford the desired product (1). Yield: 188 mg, 59%.

### Synthesis of (1R,2R)-1-((2R,3R,4S,6R)-4-acetoxy-3-(2-acetoxyacetamido)-6-(methoxycarbonyl)-6-((6-(1-(2-(2-methoxyethoxy)ethyl)-1H-1,2,3-triazol-4-yl)hexyl)oxy)tetrahydro-2H-pyran-2-yl)-3-(2-(4-ethynylphenyl)acetamido)propane-1,2-diyl diacetate (2)

To a stirred solution of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-ethynylphenyl)acetamido) propane-1,2-diyl diacetate (Int. C, 0.192 g, 254 umol) and 6-(1-(2-(2-methoxyethoxy)ethyl)-1H-1,2,3-triazol-4-yl)hexan-1-ol (1, 0.138 g, 509 umol) in anhydrous dichloromethane (3.39 mL) and anhydrous acetonitrile (5.09 mL), silver(1+) trifluoromethanesulfonate (0.163 g, 636 umol)) and activated 4 Å powdered molecular sieves (600 mg) were added, and the solution was stirred in the dark at room temperature for 1 h under nitrogen atmosphere. The solution was cooled to −78° C., and a solution of iodine monobromide (78.9 mg, 382 umol) in dichloromethane (0.382 mL) was added dropwise. The mixture was stirred at the same temperature for 3 h. The reaction mixture was quenched with N,N-diisopropylethylamine (260 uL) and allowed to warm to room temperature. The reaction mixture was filtered, and the filtrate was washed with an aq. sat. solution of sodium bicarbonate. The organic phase was dried, filtered, and concentrated. The crude residue obtained was purified by column chromatography using ethyl acetate and hexane as eluents to afford the desired product (2). Yield: 0.169 g, 74%; LCMS m/z 900.50 [M−H]⁻.

### Synthesis of (2R,4S,5R,6R)-6-((1R,2R)-3-(2-(4-ethynylphenyl)acetamido)-1,2-dihydroxypropyl)-4-hydroxy-5-(2-hydroxyacetamido)-2-((6-(1-(2-(2-methoxyethoxy)ethyl)-1H-1,2,3-triazol-4-yl)hexyl)oxy)tetrahydro-2H-pyran-2-carboxylic acid (Cpd. 27)

(1R,2R)-1-((2R,3R,4S,6R)-4-acetoxy-3-(2-acetoxyacetamido)-6-(methoxycarbonyl)-6-((6-(1-(2-(2-methoxyethoxy)ethyl)-1H-1,2,3-triazol-4-yl)hexyl)oxy)tetrahydro-2H-pyran-2-yl)-3-(2-(4-ethynylphenyl)acetamido)propane-1,2-diyl diacetate (2, 0.169 g, 0.187 mmol) in methanol (1.12 ml) was added a solution of lithium hydroxide monohydrate (0.0269 g, 1.12 mmol) in water (0.125 ml). The reaction mixture was stirred at room temperature for 6 h. After completion, the reaction mixture was treated with acidic resin (Dowex 50, H+) to pH˜6 and the suspension was filtered. The filtrate was concentrated under reduced pressure and the crude residue obtained was purified by prep HPLC using acetonitrile and water (+0.1% TFA) as eluents to afford the desired product (Cpd. 27). Yield: 121.0 mg, 91%; LCMS (ESI) m/z 720.40 [M+H]⁺. ¹H NMR (500 MHz, DMSO-d₆): 7.99 (t, J=5.5 Hz, 1H), 7.85 (d, J=7.5 Hz, 1H), 7.80 (s, 1H), 7.38 (d, J=8.0 Hz, 2H), 7.26 (d, J=8.0 Hz, 2H), 4.45 (t, J=5.5 Hz, 2H), 4.11 (s, 1H), 3.87 (m, 2H), 3.79-3.76 (t, J=5.5 Hz, 2H), 3.68-3.59 (m, 5H), 3.56-3.54 (m, 2H), 3.51-3.49 (m, 2H), 3.46 (br, 2H), 3.40-3.38 (m, 2H), 3.31-3.27 (m, 1H), 3.21 (m, 1H), 3.20 (s, 3H), 2.96-2.91 (m, 1H), 2.59 (t, J=7.5 Hz, 2H), 1.58-1.54 (m, 2H), 1.52 (dd, J=12.0 Hz, 1H), 1.49-1.44 (m, 2H), 1.31-1.28 (m, 4H).

### Compound 28

### (2R,4S,5R,6R)-6-((1R,2R)-3-(2-(4-ethynylphenyl)acetamido)-1,2-dihydroxypropyl)-4-hydroxy-5-(2-hydroxyacetamido)-2-((6-(4-((2-methoxyethoxy)methyl)-1H-1,2,3-triazol-1-yl)hexyl)oxy)tetrahydro-2H-pyran-2-carboxylic acid (Cpd. 28)

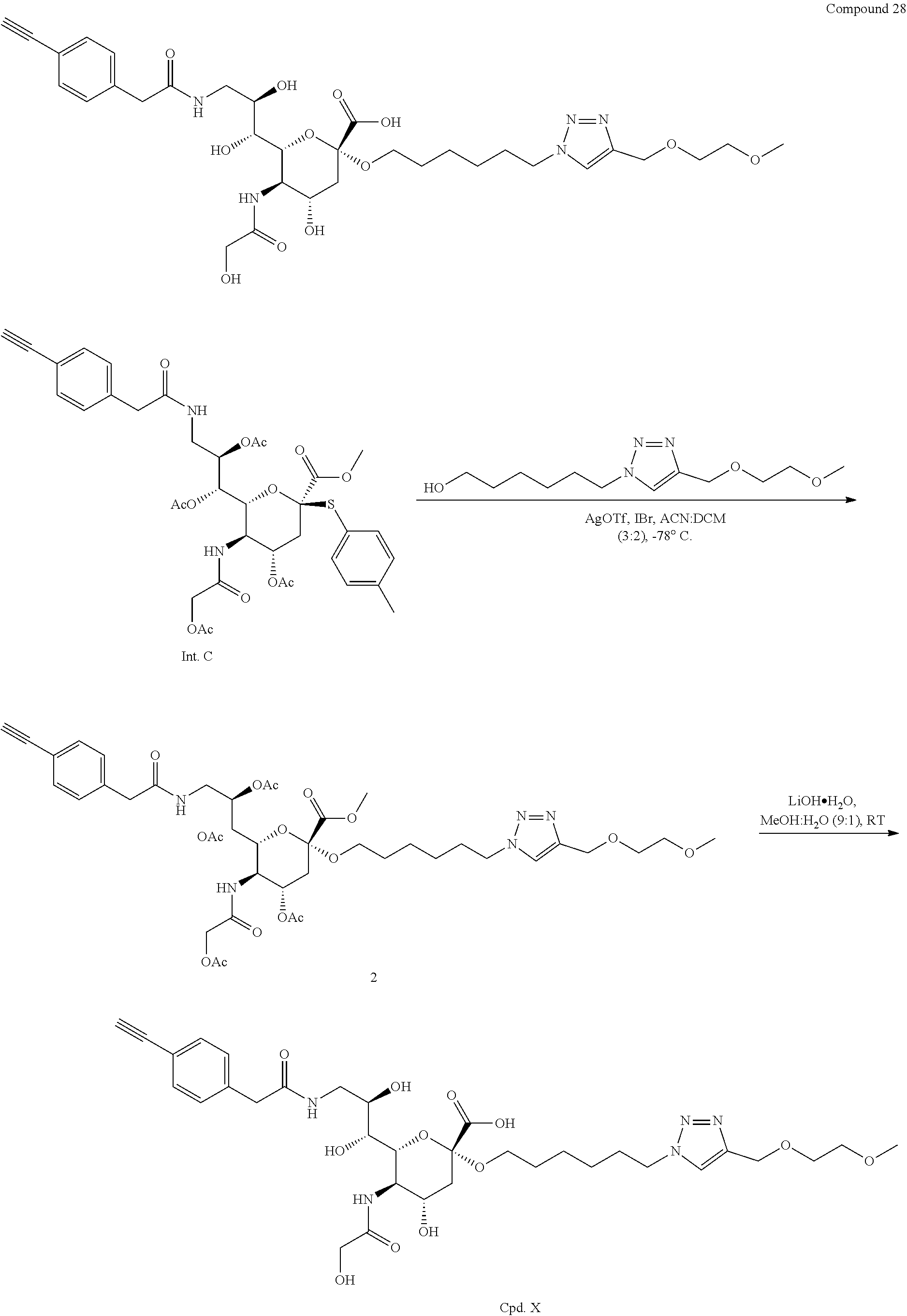

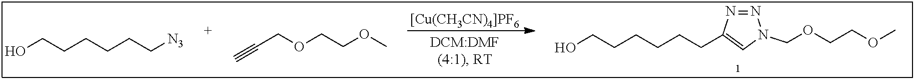

### Synthesis of 6-(4-((2-methoxyethoxy)methyl)-1H-1,2,3-triazol-1-yl)hexan-1-ol (1)

To a solution of 6-azidohexan-1-ol (104 mg, 724 umol) in anhydrous DCM (3.86 mL) and DMF (965 uL), 3-(2-methoxyethoxy)prop-1-yne (82.6 mg, 724 umol) was added followed by tetrakis(acetonitrile)copper(l) hexafluorophosphate (0.405 g, 1.09 mmol). The mixture was stirred for 45 min at room temperature, then diluted with DCM (50 mL) and washed with an aq. solution of Na₄EDTA (0.1 M, 3×50 mL). The organic phase was dried, filtered, and concentrated. The crude residue obtained was purified by column chromatography using ethyl acetate as eluent to afford the desired product (1). Yield: 138 mg, 74%.

### Synthesis of (1R,2R)-1-((2R,3R,4S,6R)-4-acetoxy-3-(2-acetoxyacetamido)-6-(methoxycarbonyl)-6-((6-(4-((2-methoxyethoxy)methyl)-1H-1,2,3-triazol-1-yl)hexyl)oxy)tetrahydro-2H-pyran-2-yl)-3-(2-(4-ethynylphenyl)acetamido)propane-1,2-diyl diacetate (2)

To a stirred solution of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-ethynylphenyl)acetamido) propane-1,2-diyl diacetate (Int. C, 0.150 g, 199 umol) and 6-(4-((2-methoxyethoxy)methyl)-1H-1,2,3-triazol-1-yl)hexan-1-ol (1, 0.102 g, 397 umol) in anhydrous dichloromethane (2.65 mL) and anhydrous acetonitrile (3.97 mL), silver(1+) trifluoromethanesulfonate (0.128 g, 497 umol)) and activated 4 Å powdered molecular sieves (500 mg) were added, and the solution was stirred in the dark at room temperature for 1 h under nitrogen atmosphere. The solution was cooled to −78° C., and a solution of iodine monobromide (61.6 mg, 298 umol) in dichloromethane (0.298 mL) was added dropwise. The mixture was stirred at the same temperature for 3 h. The reaction mixture was quenched with N,N-diisopropylethylamine (200 uL) and allowed to warm to room temperature. The reaction mixture was filtered, and the filtrate was washed with an aq. sat. solution of sodium bicarbonate. The organic phase was dried, filtered, and concentrated. The crude residue obtained was purified by column chromatography using ethyl acetate and hexane as eluents to afford the desired product (2). Yield: 0.121 g, 69%; LCMS m/z 886.50 [M−H]⁻.

### Synthesis of (2R,4S,5R,6R)-6-((1R,2R)-3-(2-(4-ethynylphenyl)acetamido)-1,2-dihydroxypropyl)-4-hydroxy-5-(2-hydroxyacetamido)-2-((6-(4-((2-methoxyethoxy)methyl)-1H-1,2,3-triazol-1-yl)hexyl)oxy)tetrahydro-2H-pyran-2-carboxylic acid (Cpd. 28)

(1R,2R)-1-((2R,3R,4S,6R)-4-acetoxy-3-(2-acetoxyacetamido)-6-(methoxycarbonyl)-6-((6-(4-((2-methoxyethoxy)methyl)-1H-1,2,3-triazol-1-yl)hexyl)oxy)tetrahydro-2H-pyran-2-yl)-3-(2-(4-ethynylphenyl)acetamido)propane-1,2-diyl diacetate (2, 0.121 g, 0.136 mmol) in methanol (0.82 ml) was added a solution of lithium hydroxide monohydrate (19.6 mg, 0.82 mmol) in water (0.091 ml). The reaction mixture was stirred at room temperature for 6 h. After completion, the reaction mixture was treated with acidic resin (Dowex 50, H+) to pH˜6 and the suspension was filtered. The filtrate was concentrated under reduced pressure and the crude residue obtained was purified by prep HPLC using acetonitrile and water (+0.1% TFA) as eluents to afford the desired product (Cpd. 28). Yield: 51.0 mg, 53%; LCMS (ESI) m/z 706.30 [M+H]⁺. ¹H NMR (500 MHz, DMSO-d₆): 8.07 (s, 1H), 8.00 (t, J=5.5 Hz, 1H), 7.84 (d, J=7.5 Hz, 1H), 7.38 (d, J=8.0 Hz, 2H), 7.26 (d, J=8.0 Hz, 2H), 4.50 (s, 2H), 4.31 (t, J=7.0 Hz, 2H), 4.11 (s, 1H), 3.88-3.86 (m, 2H), 3.83-3.78 (br, 1H), 3.67-3.59 (m, 2H), 3.56-3.53 (m, 5H), 3.46-3.43 (m, 4H), 3.30-3.26 (m, 2H), 3.22 (s, 3H), 3.21-3.20 (m, 1H), 2.96-2.90 (m, 1H), 1.81-1.76 (m, 2H), 1.51 (dd, J=12.0 Hz, 1H), 1.45-1.41 (m, 2H), 1.45-1.41 (m, 2H), 1.33-1.25 (m, 2H), 1.23-1.19 (m, 2H).

### Compound 29

### (2R,4S,5R,6R)-6-((1R,2R)-3-(2-(4-ethynylphenyl)acetamido)-1,2-dihydroxypropyl)-4-hydroxy-5-(2-hydroxyacetamido)-2-((6-pentanamidohexyl)oxy)tetrahydro-2H-pyran-2-carboxylic acid (Cpd. 29)

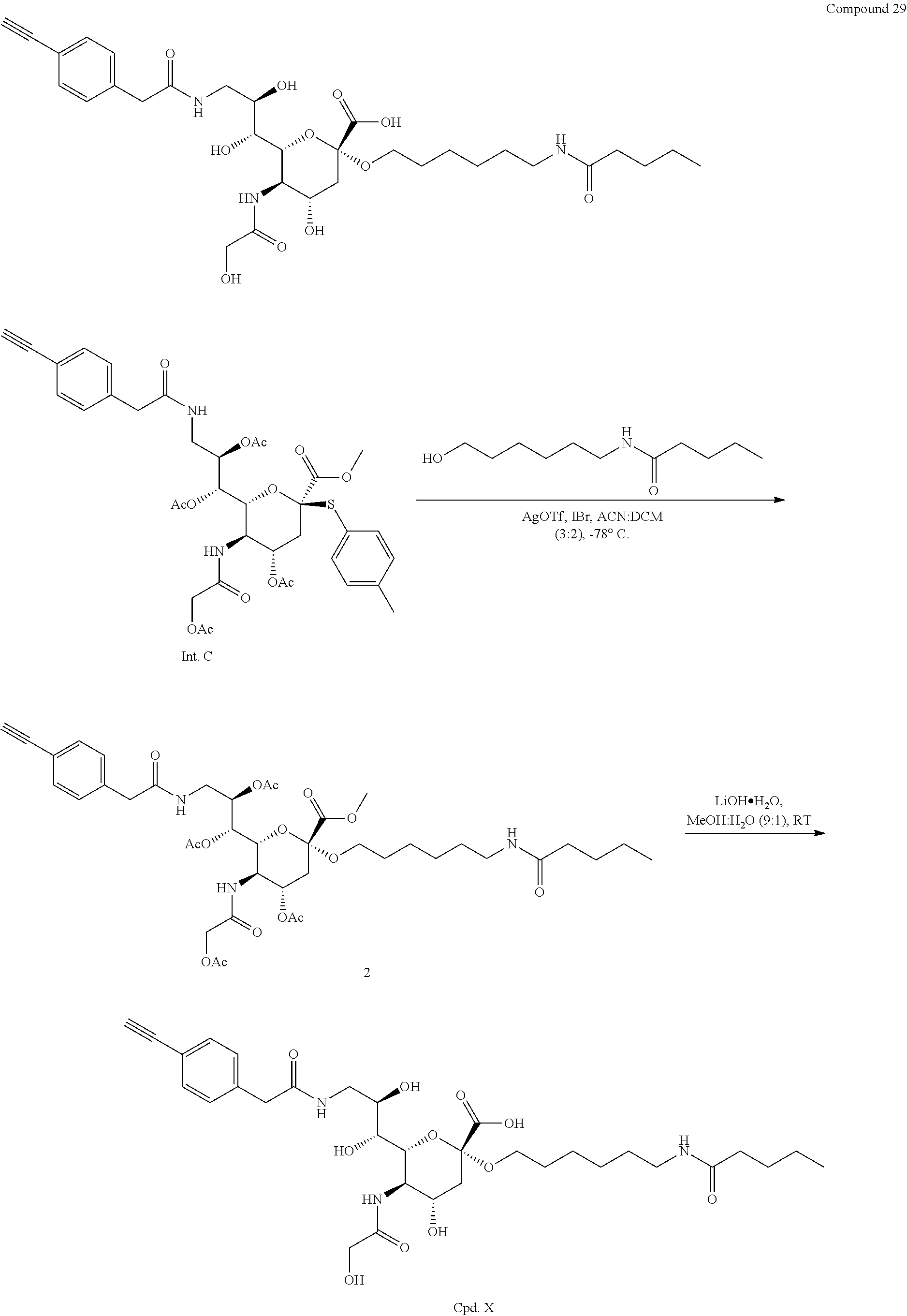

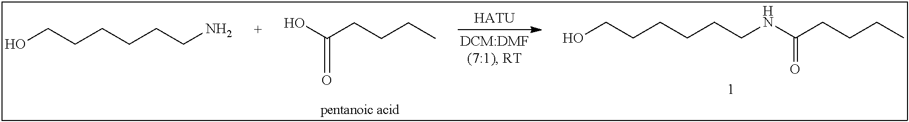

### Synthesis of N-(6-hydroxyhexyl)pentanamide (1)

To a solution of 6-aminohexan-1-ol (200 mg, 1.71 mmol) and pentanoic acid (183 mg, 1.79 mmol) in anhydrous DCM (15.0 mL) and DMF (2.0 mL), HATU (0.780 g, 2.05 mmol) was added followed by N,N-diisopropylethylamine (0.892 mL, 5.12 mmol). The mixture was stirred for 1 h at room temperature, and then filtered. The filtrate was concentrated under reduced pressure and the crude residue obtained was purified by prep HPLC using acetonitrile and water (+0.1% TFA) as eluents to afford the desired product (1). Yield: 314 mg, 91%.

### Synthesis of (1R,2R)-1-((2R,3R,4S,6R)-3-(2-acetoxyacetamido)-4-hydroxy-6-(methoxycarbonyl)-6-((6-pentanamidohexyl)oxy)tetrahydro-2H-pyran-2-yl)-3-(2-(4-ethynylphenyl)acetamido) propane-1,2-diyl diacetate (2)

To a stirred solution of (1R,2R)-1-((2R,3R,4S,6R)-4-acetoxy-3-(2-acetoxyacetamido)-6-(methoxycarbonyl)-6-(p-tolylthio)tetrahydro-2H-pyran-2-yl)-3-(2-(4-ethynylphenyl)acetamido) propane-1,2-diyl diacetate (Int. C, 0.150 g, 199 umol) and N-(6-hydroxyhexyl)pentanamide (1, 0.100 g, 497 umol) in anhydrous dichloromethane (2.65 mL) and anhydrous acetonitrile (3.97 mL), silver(1+) trifluoromethanesulfonate (0.102 g, 397 umol)) and activated 4 Å powdered molecular sieves (500 mg) were added, and the solution was stirred in the dark at room temperature for 1 h under nitrogen atmosphere. The solution was cooled to −78° C., and a solution of iodine monobromide (61.6 mg, 298 umol) in dichloromethane (0.298 mL) was added dropwise. The mixture was stirred at the same temperature for 3 h. The reaction mixture was quenched with N,N-diisopropylethylamine (200 uL) and allowed to warm to room temperature. The reaction mixture was filtered, and the filtrate was washed with an aq. sat. solution of sodium bicarbonate. The organic phase was dried, filtered, and concentrated. The crude residue obtained was purified by column chromatography using ethyl acetate and hexane as eluents to afford the desired product (2). Yield: 0.113 g, 68%; LCMS m/z 832.40 [M+H]⁺.

### Synthesis of (2R,4S,5R,6R)-6-((1R,2R)-3-(2-(4-ethynylphenyl)acetamido)-1,2-dihydroxypropyl)-4-hydroxy-5-(2-hydroxyacetamido)-2-((6-pentanamidohexyl)oxy)tetrahydro-2H-pyran-2-carboxylic acid (Cpd. 29)

(1R,2R)-1-((2R,3R,4S,6R)-3-(2-acetoxyacetamido)-4-hydroxy-6-(methoxycarbonyl)-6-((6-pentanamidohexyl)oxy)tetrahydro-2H-pyran-2-yl)-3-(2-(4-ethynylphenyl)acetamido)propane-1,2-diyl diacetate (2, 0.113 g, 0.136 mmol) in methanol (0.82 ml) was added a solution of lithium hydroxide monohydrate (19.5 mg, 0.82 mmol) in water (0.091 ml). The reaction mixture was stirred at room temperature for 6 h. After completion, the reaction mixture was treated with acidic resin (Dowex 50, H+) to pH˜6 and the suspension was filtered. The filtrate was concentrated under reduced pressure and the crude residue obtained was purified by prep HPLC using acetonitrile and water (+0.1% TFA) as eluents to afford the desired product (Cpd. 29). Yield: 53.0 mg, 60%; LCMS (ESI) m/z 650.30 [M+H]⁺. ¹H NMR (500 MHz, DMSO-d6): 8.00 (t, J=5.5 Hz, 1H), 7.84 (d, J=7.5 Hz, 1H), 7.70 (t, J=5.5 Hz, 1H), 7.38 (d, J=8.0 Hz, 2H), 7.26 (d, J=8.0 Hz, 2H), 5.54 (br, 1H), 4.94 (br, 1H), 4.71 (br, 1H), 4.28 (br, 1H), 4.11 (s, 1H), 3.87 (m, 2H), 3.81-3.78 (br, 1H), 3.68-3.59 (m, 2H), 3.57-3.51 (m, 3H), 3.47 (m, 2H), 3.27-3.26 (m, 1H), 3.21-3.19 (m, 2H), 3.02-2.97 (m, 2H), 2.96-2.91 (m, 1H), 2.03 (t, J=7.5 Hz, 2H), 1.52 (dd, J=12.0 Hz, 1H), 1.48-1.42 (m, 4H), 1.37-1.34 (m, 2H), 1.27-1.20 (m, 6H), 0.85 (t, J=7.5 Hz, 3H).

### Compound 30

### (2S,4S,5R,6R)-2-(benzylthio)-6-((1R,2R)-1,2-dihydroxy-3-(2-(4-(1-(15-oxo-15-(perfluorophenoxy)-3,6,9,12-tetraoxapentadecyl)-1H-1,2,3-triazol-4-yl)phenyl)acetamido)propyl)-4-hydroxy-5-(2-hydroxyacetamido)tetrahydro-2H-pyran-2-carboxylic acid (Cpd. 30)

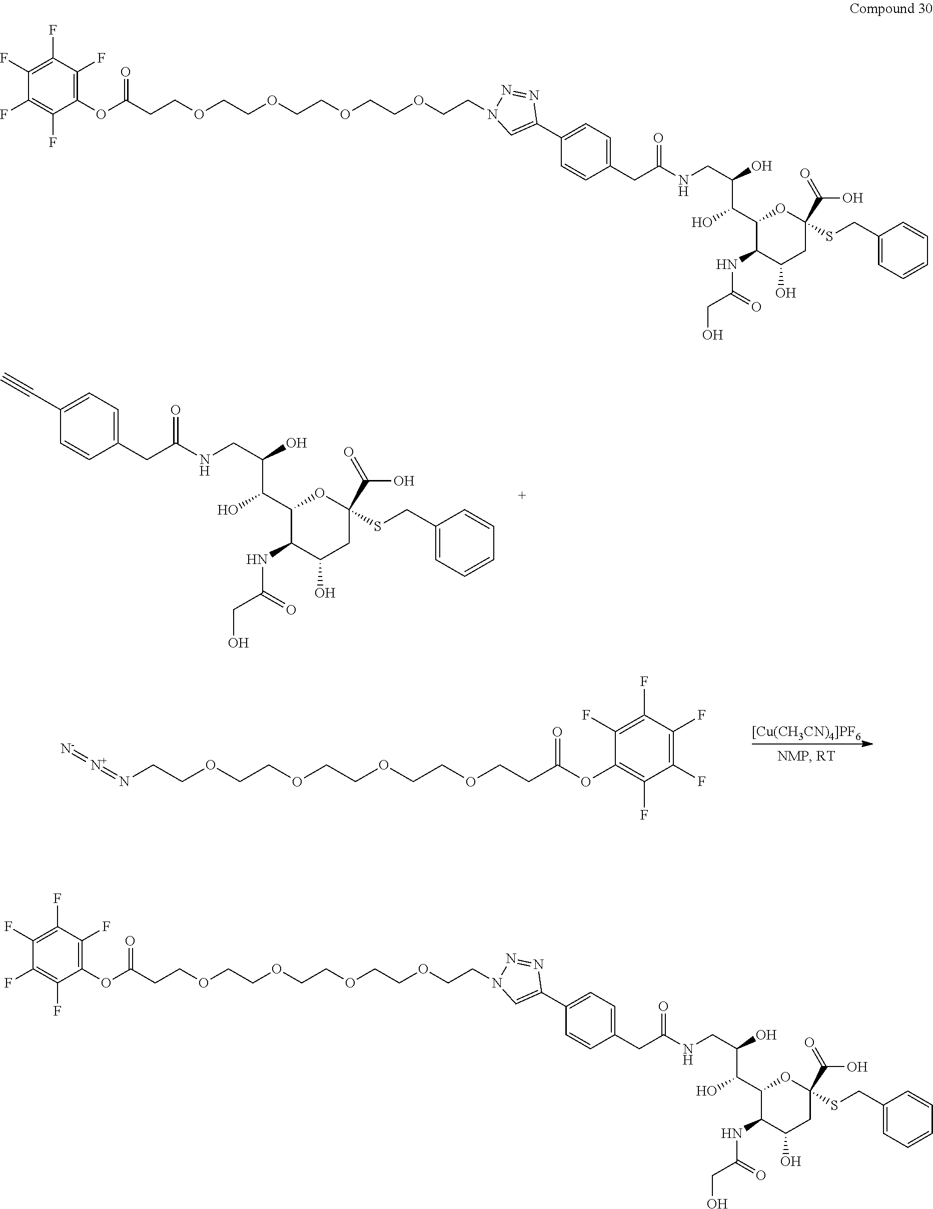

### Synthesis of (2S,4S,5R,6R)-2-(benzylthio)-6-((1R,2R)-1,2-dihydroxy-3-(2-(4-(1-(15-oxo-15-(perfluorophenoxy)-3,6,9,12-tetraoxapentadecyl)-1H-1,2,3-triazol-4-yl)phenyl)acetamido)propyl)-4-hydroxy-5-(2-hydroxyacetamido)tetrahydro-2H-pyran-2-carboxylic acid (Cpd. 30)

To a stirred solution of (2S,4S,5R,6R)-2-(benzylthio)-6-((1R,2R)-3-(2-(4-ethynylphenyl)acetamido)-1,2-dihydroxypropyl)-4-hydroxy-5-(2-hydroxyacetamido)tetrahydro-2H-pyran-2-carboxylic acid (15.3 mg, 26.7 umol) in anhydrous NMP (356 uL) was added Azido-PEG4-PFP-ester (13.4 mg, 29.4 umol), followed by Tetrakis(acetonitrile)copper(l) hexafluorophosphate (24.9 mg, 66.8 umol). The vial was sealed, and the mixture was stirred for 1 h at room temperature. The mixture was filtered through a 0.45 μm PTFE filter. A few drops of acetic acid were added to the filtrate. The filtrate was purified by preparatory HPLC using acetonitrile and water (+0.05% TFA) as eluents. The fractions containing the desired product were lyophilized to afford the desired product (Cpd. 30) as a white solid. Yield: 16.1 mg, 59%; LCMS (ESI) m/z 1030.30 [M+H]⁺. ¹H NMR (500 MHz, DMSO-d₆): 8.41 (s, 1H), 7.97 (t, J=5.5 Hz, 1H), 7.90 (d, J=8.0 Hz, 1H), 7.70 (d, J=8.5 Hz, 2H), 7.30 (d, J=8.5 Hz, 2H), 7.28 (d, J=8.5 Hz, 2H), 7.28 (m, 2H), 7.21 (m, 1H), 5.54 (t, J=5.5 Hz, 1H), 5.03 (br, 1H), 4.77 (d, J=5.0 Hz, 1H), 4.55 (t, J=5.0 Hz, 2H), 3.96-3.94 (m, 1H), 3.89-3.82 (m, 6H), 3.73 (t, J=5.5 Hz, 2H), 3.73-3.69 (m, 1H), 3.61-3.53 (m, 5H), 3.51-3.44 (m, 12H), 3.23-3.21 (m, 1H), 3.00 (t, J=6.0 Hz, 2H), 2.96-2.91 (m, 1H), 2.64-2.58 (m, 1H), 1.60 (dd, J=12.0 Hz, 1H).

### Compound 31

### (2R,4S,5R,6R)-6-((1R,2R)-1,2-dihydroxy-3-(2-(4-(1-(15-oxo-15-(perfluorophenoxy)-3,6,9,12-tetraoxapentadecyl)-1H-1,2,3-triazol-4-yl)phenyl)acetamido)propyl)-4-hydroxy-5-(2-hydroxyacetamido)-2-((6-methoxyhexyl)oxy)tetrahydro-2H-pyran-2-carboxylic acid (Cpd. 31)

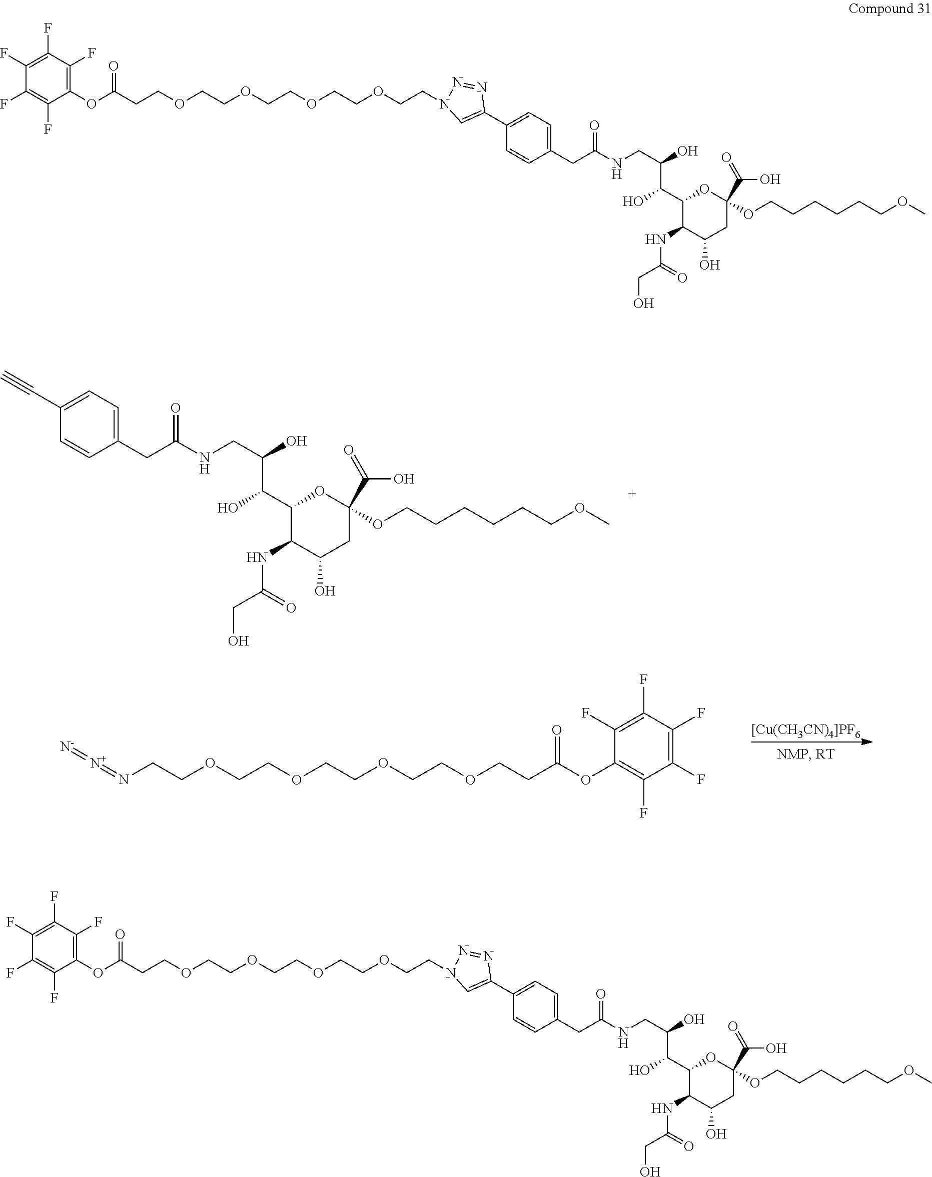

### Synthesis of (2R,4S,5R,6R)-6-((1R,2R)-1,2-dihydroxy-3-(2-(4-(1-(15-oxo-15-(perfluorophenoxy)-3,6,9,12-tetraoxapentadecyl)-1H-1,2,3-triazol-4-yl)phenyl)acetamido)propyl)-4-hydroxy-5-(2-hydroxyacetamido)-2-((6-methoxyhexyl)oxy)tetrahydro-2H-pyran-2-carboxylic acid (Cpd. 31)

To a stirred solution of (2R,4S,5R,6R)-6-((1R,2R)-3-(2-(4-ethynylphenyl)acetamido)-1,2-dihydroxypropyl)-4-hydroxy-5-(2-hydroxyacetamido)-2-((6-methoxyhexyl)oxy)tetrahydro-2H-pyran-2-carboxylic acid (19.1 mg, 32.9 umol) in anhydrous NMP (439 uL) was added Azido-PEG4-PFP-ester (16.5 mg, 36.2 umol), followed by Tetrakis(acetonitrile)copper(l) hexafluorophosphate (30.7 mg, 82.2 umol). The vial was sealed, and the mixture was stirred for 1 h at room temperature. The mixture was filtered through a 0.45 μm PTFE filter. A few drops of acetic acid were added to the filtrate. The filtrate was purified by preparatory HPLC using acetonitrile and water (+0.05% TFA) as eluents. The fractions containing the desired product were lyophilized to afford (Cpd. 31) as a white solid. Yield: 21.1 mg, 62%; LCMS (ESI) m/z 1038.40 [M+H]⁺. ¹H NMR (500 MHz, DMSO-d6): 8.46 (s, 1H), 7.96 (t, J=5.5 Hz, 1H), 7.82 (d, J=8.0 Hz, 1H), 7.73 (d, J=8.5 Hz, 2H), 7.32 (d, J=8.5 Hz, 2H), 5.52 (t, J=5.5 Hz, 1H), 4.91 (br, 1H), 4.70 (d, J=5.0 Hz, 1H), 4.55 (t, J=5.0 Hz, 2H), 3.91-3.83 (m, 4H), 3.81-3.76 (m, 1H), 3.73 (t, J=6.0 Hz, 2H), 3.69-3.65 (m, 1H), 3.64-3.59 (m, 2H), 3.57-3.44 (m, 18H), 3.26 (t, J=6.5 Hz, 3H), 3.20-3.18 (m, 1H), 3.19 (s, 3H), 3.00 (t, J=6.0 Hz, 2H), 2.95-2.90 (m, 1H), 2.54-2.51 (m, 1H), 1.46-1.42 (m, 4H), 1.26-1.23 (m, 4H).

### Compound 32

### (2R,4S,5R,6R)-6-((1R,2R)-1,2-dihydroxy-3-(2-(4-(1-(15-oxo-15-(perfluorophenoxy)-3,6,9,12-tetraoxapentadecyl)-1H-1,2,3-triazol-4-yl)phenyl)acetamido)propyl)-2-(hexyloxy)-4-hydroxy-5-(2-hydroxyacetamido)tetrahydro-2H-pyran-2-carboxylic acid (Cpd. 32)

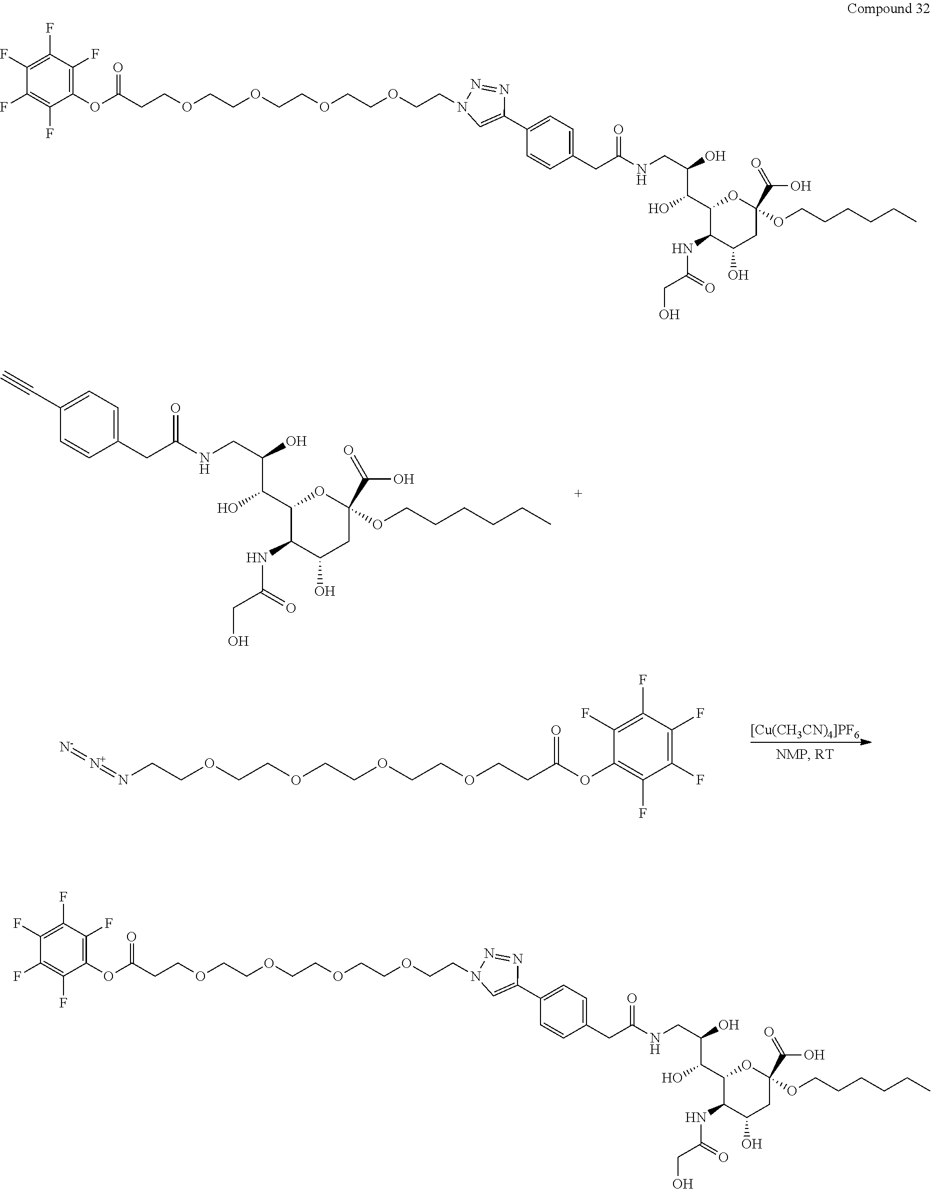

### Synthesis of (2R,4S,5R,6R)-6-((1R,2R)-1,2-dihydroxy-3-(2-(4-(1-(15-oxo-15-(perfluorophenoxy)-3,6,9,12-tetraoxapentadecyl)-1H-1,2,3-triazol-4-yl)phenyl)acetamido)propyl)-2-(hexyloxy)-4-hydroxy-5-(2-hydroxyacetamido)tetrahydro-2H-pyran-2-carboxylic acid (Cpd. 32)

To a stirred solution of (2R,4S,5R,6R)-6-((1R,2R)-3-(2-(4-ethynylphenyl)acetamido)-1,2-dihydroxypropyl)-2-(hexyloxy)-4-hydroxy-5-(2-hydroxyacetamido)tetrahydro-2H-pyran-2-carboxylic acid (7.11 mg, 12.9 umol) in anhydrous NMP (172 uL) was added Azido-PEG4-PFP-ester (6.50 mg, 14.2 umol), followed by Tetrakis(acetonitrile)copper(l) hexafluorophosphate (12.0 mg, 32.3 umol). The vial was sealed, and the mixture was stirred for 1 h at room temperature. The mixture was filtered through a 0.45 μm PTFE filter. A few drops of acetic acid were added to the filtrate. The filtrate was purified by preparatory HPLC using acetonitrile and water (+0.05% TFA) as eluents. The fractions containing the desired product were lyophilized to afford Cpd. 32 as a white solid. Yield: 7.1 mg, 55%; LCMS (ESI) m/z 1008.40 [M+H]⁺. ¹H NMR (500 MHz, DMSO-d₆): 8.46 (s, 1H), 7.96 (t, J=5.5 Hz, 1H), 7.84 (d, J=7.0 Hz, 1H), 7.73 (d, J=8.5 Hz, 2H), 7.32 (d, J=8.5 Hz, 2H), 5.53 (t, J=5.5 Hz, 1H), 4.93 (d, J=5.5 Hz, 1H), 4.71 (d, J=5.0 Hz, 1H), 4.55 (t, J=5.0 Hz, 2H), 3.91-3.83 (m, 4H), 3.81-3.76 (m, 1H), 3.73 (t, J=6.0 Hz, 2H), 3.69-3.65 (m, 1H), 3.64-3.60 (m, 1H), 3.58-3.53 (m, 4H), 3.52-3.45 (m, 13H), 3.29-3.27 (m, 1H), 3.22-3.19 (m, 1H), 3.00 (t, J=6.0 Hz, 2H), 2.95-2.90 (m, 1H), 2.53-2.50 (m, 1H), 1.49 (t, J=12.0 Hz, 1H), 1.46-1.42 (m, 2H), 1.25-1.22 (m, 6H), 0.83 (t, J=7.0 Hz, 3H).

### Compound 33

### (2R,4S,5R,6R)-6-((1R,2R)-1,2-dihydroxy-3-(2-(4-(1-(15-oxo-15-(perfluorophenoxy)-3,6,9,12-tetraoxapentadecyl)-1H-1,2,3-triazol-4-yl)phenyl)acetamido)propyl)-4-hydroxy-5-(2-hydroxyacetamido)-2-(2-(2-(2-methoxyethoxy)ethoxy)ethoxy)tetrahydro-2H-pyran-2-carboxylic acid (Cpd. 33)

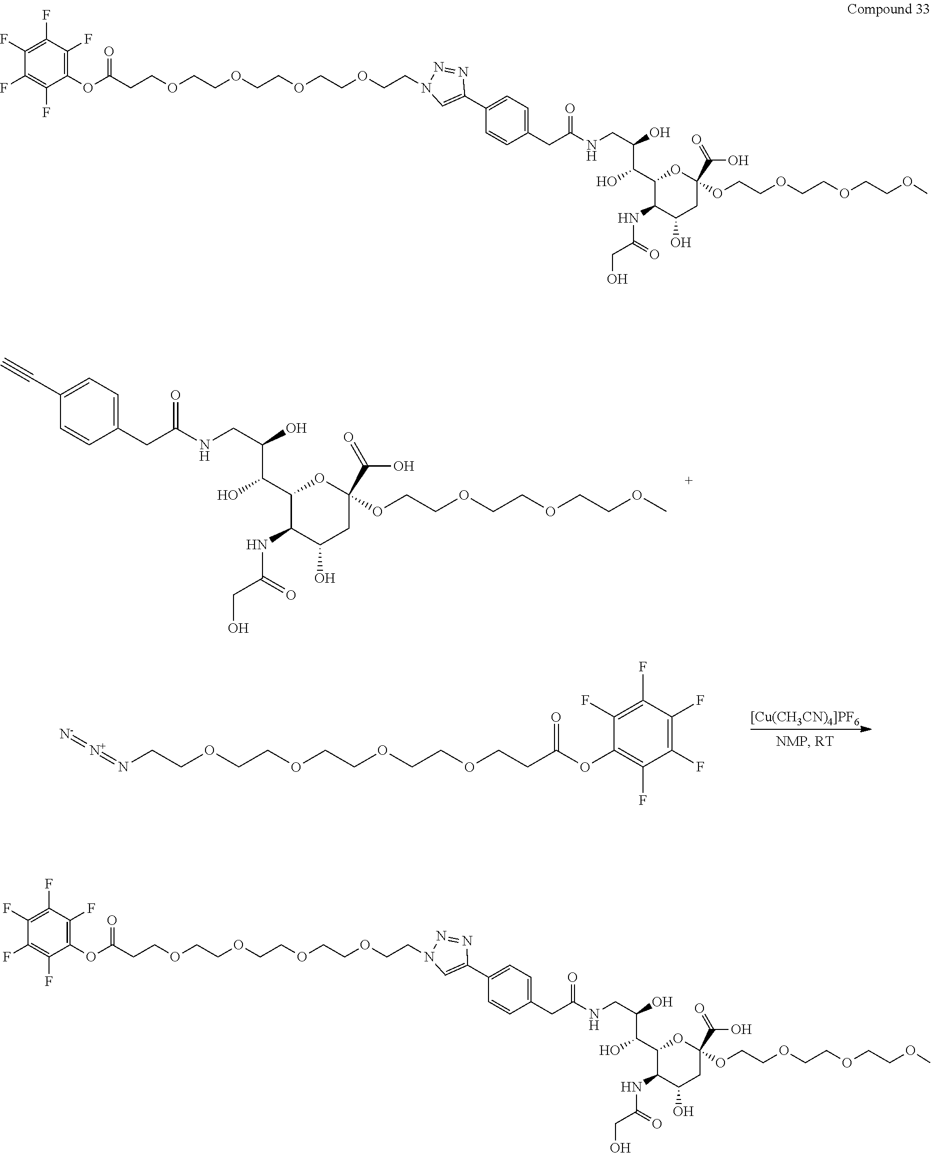

### Synthesis of (2R,4S,5R,6R)-6-((1R,2R)-1,2-dihydroxy-3-(2-(4-(1-(15-oxo-15-(perfluorophenoxy)-3,6,9,12-tetraoxapentadecyl)-1H-1,2,3-triazol-4-yl)phenyl)acetamido)propyl)-4-hydroxy-5-(2-hydroxyacetamido)-2-(2-(2-(2-methoxyethoxy)ethoxy)ethoxy)tetrahydro-2H-pyran-2-carboxylic acid (Cpd. 33)

To a stirred solution of (2R,4S,5R,6R)-6-((1R,2R)-3-(2-(4-ethynylphenyl)acetamido)-1,2-dihydroxypropyl)-4-hydroxy-5-(2-hydroxyacetamido)-2-(2-(2-(2-methoxyethoxy)ethoxy)ethoxy)tetrahydro-2H-pyran-2-carboxylic acid (28.5 mg, 46.5 umol) in anhydrous NMP (620 uL) was added Azido-PEG4-PFP-ester (23.4 mg, 51.2 umol), followed by Tetrakis(acetonitrile)copper(l) hexafluorophosphate (43.3 mg, 116 umol). The vial was sealed, and the mixture was stirred for 1 h at room temperature. The mixture was filtered through a 0.45 μm PTFE filter. A few drops of acetic acid were added to the filtrate. The filtrate was purified by preparatory HPLC using acetonitrile and water (+0.05% TFA) as eluents. The fractions containing the desired product were lyophilized to afford Cpd. 33 as a white solid. Yield: 31.6 mg, 63%; LCMS (ESI) m/z 1070.40 [M+H]⁺. ¹H NMR (500 MHz, DMSO-d₆): 8.46 (s, 1H), 7.96 (t, J=5.5 Hz, 1H), 7.83 (d, J=7.5 Hz, 1H), 7.74 (d, J=8.0 Hz, 2H), 7.32 (d, J=8.0 Hz, 2H), 5.52 (t, J=5.5 Hz, 1H), 4.94 (d, J=5.5 Hz, 1H), 4.71 (d, J=5.0 Hz, 1H), 4.55 (t, J=5.0 Hz, 2H), 3.91-3.83 (m, 4H), 3.82-3.77 (m, 1H), 3.76-3.73 (m, 1H), 3.73 (t, J=6.0 Hz, 2H), 3.70-3.66 (m, 1H), 3.56-3.52 (m, 5H), 3.51-3.45 (m, 21H), 3.44-3.41 (m, 2H), 3.23 (s, 3H), 3.21-3.19 (m, 1H), 3.00 (t, J=6.0 Hz, 2H), 2.97-2.92 (m, 1H), 2.53 (dd, J=13.0 Hz, 5.5 Hz, 1H), 1.50 (dd, J=12.0 Hz, 1H).

### Compound 34

### (2R,4S,5R,6R)-6-((1R,2R)-1,2-dihydroxy-3-(2-(4-(1-(15-oxo-15-(perfluorophenoxy)-3,6,9,12-tetraoxapentadecyl)-1H-1,2,3-triazol-4-yl)phenyl)acetamido)propyl)-4-hydroxy-5-(2-hydroxyacetamido)-2-((6-(1-(2-(2-methoxyethoxy)ethyl)-1H-1,2,3-triazol-4-yl)hexyl)oxy)tetrahydro-2H-pyran-2-carboxylic acid (Cpd. 34)

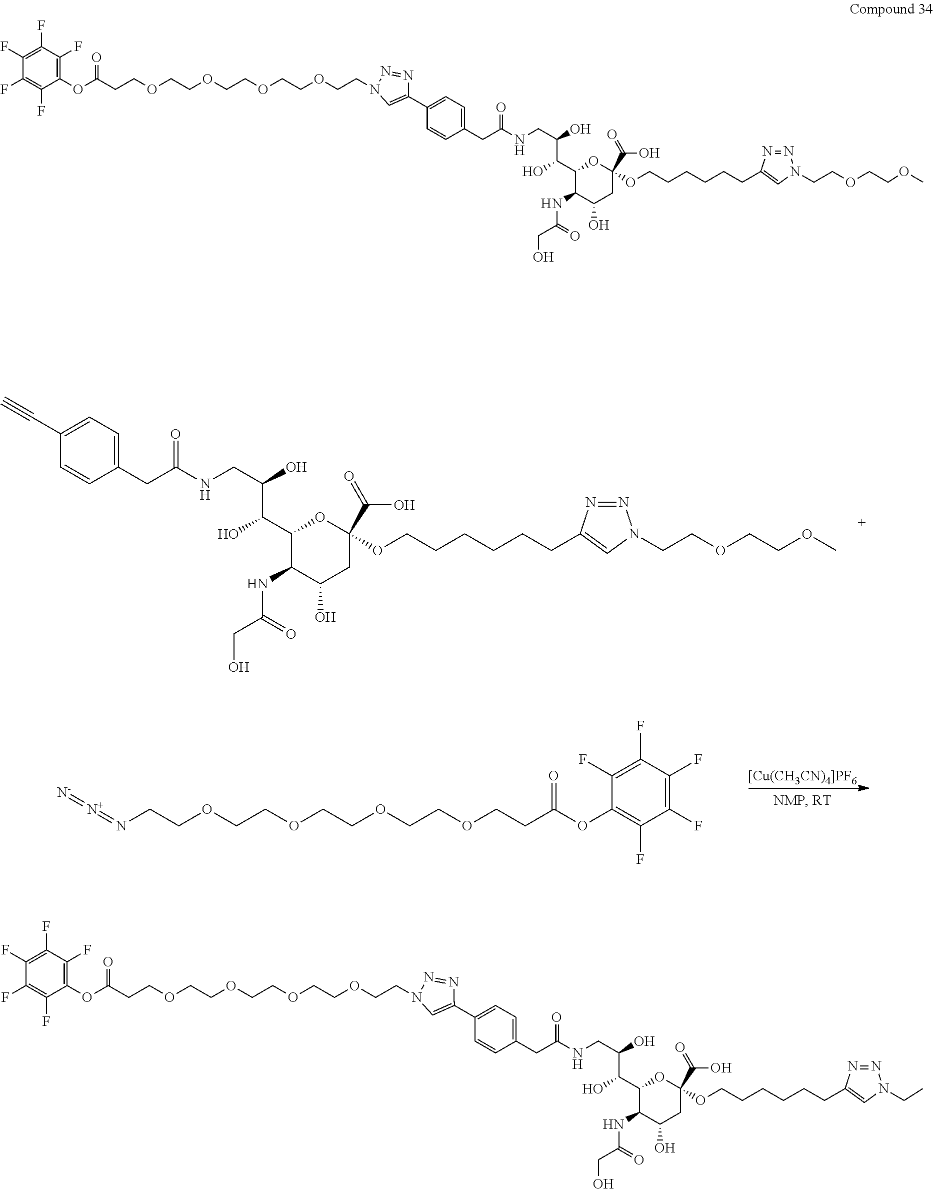

### Synthesis of (2R,4S,5R,6R)-6-((1R,2R)-1,2-dihydroxy-3-(2-(4-(1-(15-oxo-15-(perfluorophenoxy)-3,6,9,12-tetraoxapentadecyl)-1H-1,2,3-triazol-4-yl)phenyl)acetamido)propyl)-4-hydroxy-5-(2-hydroxyacetamido)-2-((6-(1-(2-(2-methoxyethoxy)ethyl)-1H-1,2,3-triazol-4-yl)hexyl)oxy)tetrahydro-2H-pyran-2-carboxylic acid (Cpd. 34)

To a stirred solution of (2R,4S,5R,6R)-6-((1R,2R)-3-(2-(4-ethynylphenyl)acetamido)-1,2-dihydroxypropyl)-4-hydroxy-5-(2-hydroxyacetamido)-2-((6-(1-(2-(2-methoxyethoxy)ethyl)-1H-1,2,3-triazol-4-yl)hexyl)oxy)tetrahydro-2H-pyran-2-carboxylic acid (39.2 mg, 54.5 umol) in anhydrous NMP (727 uL) was added Azido-PEG4-PFP-ester (27.4 mg, 60.0 umol), followed by Tetrakis(acetonitrile)copper(l) hexafluorophosphate (50.8 mg, 136 umol). The vial was sealed, and the mixture was stirred for 1 h at room temperature. The mixture was filtered through a 0.45 μm PTFE filter. A few drops of acetic acid were added to the filtrate. The filtrate was purified by preparatory HPLC using acetonitrile and water (+0.05% TFA) as eluents. The fractions containing the desired product were lyophilized to afford Cpd. 34 as a white solid. Yield: 37.8 mg, 59%; LCMS (ESI) m/z 1177.50 [M+H]⁺. ¹H NMR (500 MHz, DMSO-d₆): 8.46 (s, 1H), 7.96 (t, J=5.5 Hz, 1H), 7.84 (d, J=7.5 Hz, 1H), 7.79 (s, 1H), 7.73 (d, J=8.0 Hz, 2H), 7.31 (d, J=8.0 Hz, 2H), 5.52 (t, J=5.5 Hz, 1H), 4.94 (d, J=5.5 Hz, 1H), 4.71 (d, J=4.5 Hz, 1H), 4.55 (t, J=5.5 Hz, 2H), 4.44 (t, J=5.5 Hz, 2H), 3.91-3.83 (m, 4H), 3.82-3.77 (m, 1H), 3.77 (t, J=5.5 Hz, 2H), 3.73 (t, J=6.0 Hz, 2H), 3.70-3.66 (m, 1H), 3.65-3.61 (m, 1H), 3.56-3.52 (m, 5H), 3.51-3.44 (m, 14H), 3.40-3.38 (m, 2H), 3.30-3.28 (m, 1H), 3.22-3.20 (m, 1H), 3.20 (s, 3H), 3.00 (t, J=6.0 Hz, 2H), 2.98-2.93 (m, 1H), 2.57 (t, J=7.5 Hz, 2H), 2.52-2.50 (m, 1H), 1.58-1.52 (m, 2H), 1.51 (dd, J=12.0 Hz, 1H), 1.47-1.43 (m, 2H), 1.30-1.26 (m, 4H).

### Compound 35

### (2R,4S,5R,6R)-6-((1R,2R)-1,2-dihydroxy-3-(2-(4-(1-(15-oxo-15-(perfluorophenoxy)-3,6,9,12-tetraoxapentadecyl)-1H-1,2,3-triazol-4-yl)phenyl)acetamido)propyl)-4-hydroxy-5-(2-hydroxyacetamido)-2-((6-(4-((2-methoxyethoxy)methyl)-1H-1,2,3-triazol-1-yl)hexyl)oxy)tetrahydro-2H-pyran-2-carboxylic acid (Cpd. 35)

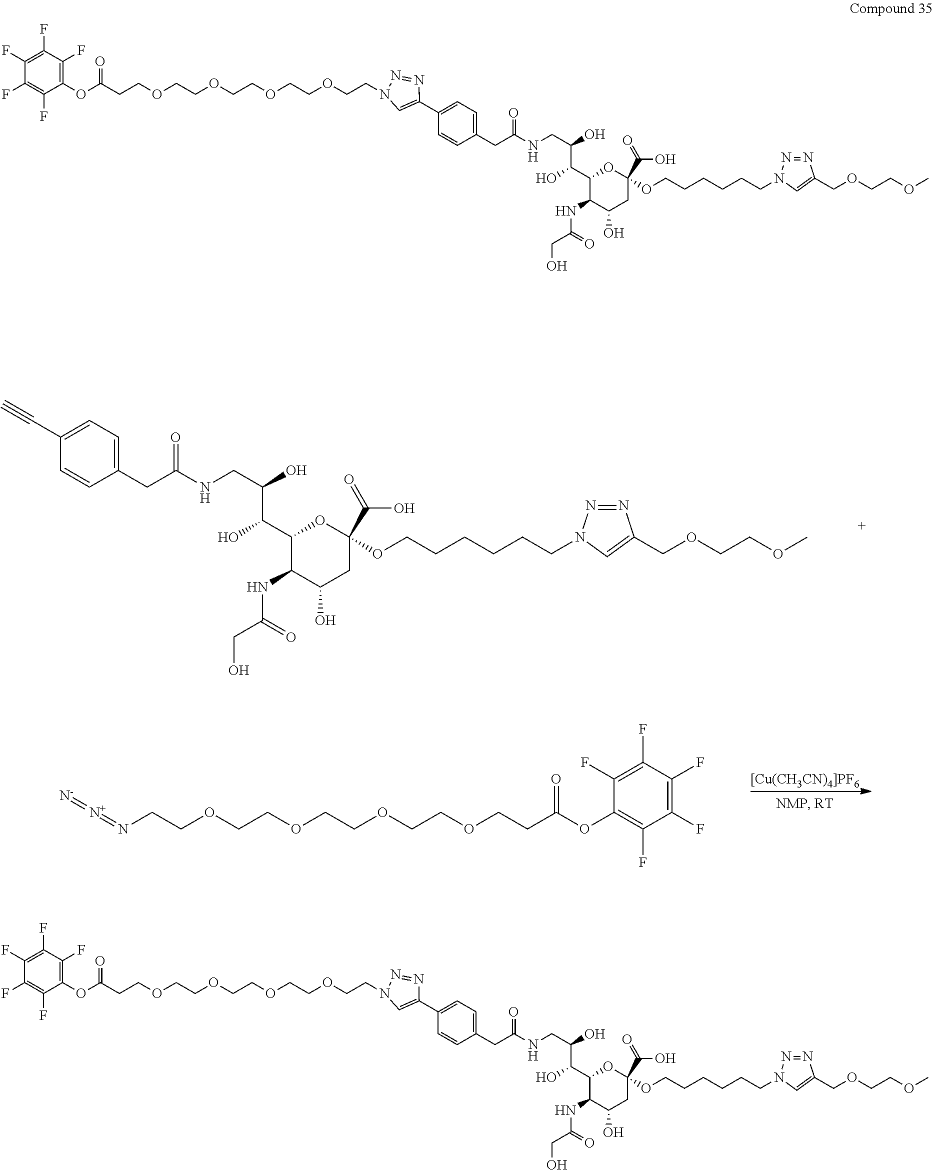

### Synthesis of (2R,4S,5R,6R)-6-((1R,2R)-1,2-dihydroxy-3-(2-(4-(1-(15-oxo-15-(perfluorophenoxy)-3,6,9,12-tetraoxapentadecyl)-1H-1,2,3-triazol-4-yl)phenyl)acetamido)propyl)-4-hydroxy-5-(2-hydroxyacetamido)-2-((6-(4-((2-methoxyethoxy)methyl)-1H-1,2,3-triazol-1-yl)hexyl)oxy)tetrahydro-2H-pyran-2-carboxylic acid (Cpd. 35)

To a stirred solution of (2R,4S,5R,6R)-6-((1R,2R)-3-(2-(4-ethynylphenyl)acetamido)-1,2-dihydroxypropyl)-4-hydroxy-5-(2-hydroxyacetamido)-2-((6-(4-((2-methoxyethoxy)methyl)-1H-1,2,3-triazol-1-yl)hexyl)oxy)tetrahydro-2H-pyran-2-carboxylic acid (27.8 mg, 39.4 umol) in anhydrous NMP (525 uL) was added Azido-PEG4-PFP-ester (19.8 mg, 43.3 umol), followed by Tetrakis(acetonitrile)copper(l) hexafluorophosphate (36.7 mg, 98.5 umol). The vial was sealed, and the mixture was stirred for 1 h at room temperature. The mixture was filtered through a 0.45 μm PTFE filter. A few drops of acetic acid were added to the filtrate. The filtrate was purified by preparatory HPLC using acetonitrile and water (+0.05% TFA) as eluents. The fractions containing the desired product were lyophilized to afford Cpd. 35 as a white solid. Yield: 22.8 mg, 50%; LCMS (ESI) m/z 1177.50 [M+H]⁺. ¹H NMR (500 MHz, DMSO-d6): 8.46 (s, 1H), 8.07 (s, 1H), 7.97 (t, J=5.5 Hz, 1H), 7.83 (d, J=7.5 Hz, 1H), 7.73 (d, J=8.0 Hz, 2H), 7.31 (d, J=8.0 Hz, 2H), 5.52 (t, J=5.5 Hz, 1H), 4.93 (br, 1H), 4.71 (d, J=4.5 Hz, 1H), 4.55 (t, J=5.0 Hz, 2H), 4.50 (s, 2H), 4.30 (t, J=7.0 Hz, 2H), 3.91-3.83 (m, 4H), 3.81-3.76 (m, 1H), 3.73 (t, J=6.0 Hz, 2H), 3.70-3.66 (m, 1H), 3.64-3.60 (m, 1H), 3.58-3.53 (m, 7H), 3.51-3.45 (m, 12H), 3.44-3.42 (m, 2H), 3.29-3.27 (m, 1H), 3.22 (s, 3H), 3.22-3.19 (m, 1H), 3.00 (t, J=6.0 Hz, 2H), 2.95-2.90 (m, 1H), 2.53-2.51 (m, 1H), 1.48 (m, 1H) 1.45-1.39 (m, 2H), 1.30-1.23 (m, 2H), 1.22-1.16 (m, 2H).

### Compound 36

### (2R,4S,5R,6R)-6-((1R,2R)-1,2-dihydroxy-3-(2-(4-(1-(15-oxo-15-(perfluorophenoxy)-3,6,9,12-tetraoxapentadecyl)-1H-1,2,3-triazol-4-yl)phenyl)acetamido)propyl)-4-hydroxy-5-(2-hydroxyacetamido)-2-((6-((2-((2-methoxyethyl)amino)-3,4-dioxocyclobut-1-en-1-yl)amino)hexyl)oxy)tetrahydro-2H-pyran-2-carboxylic acid (Cpd. 36)

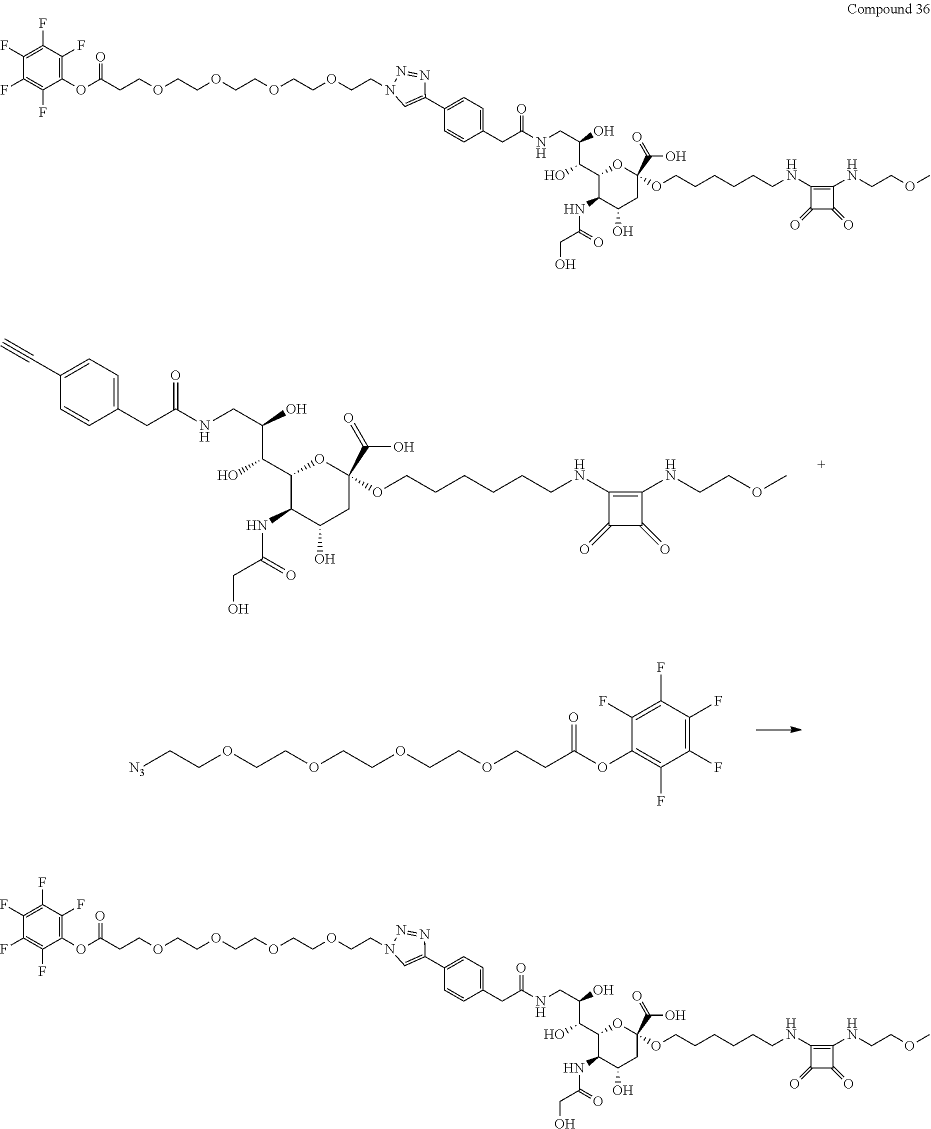

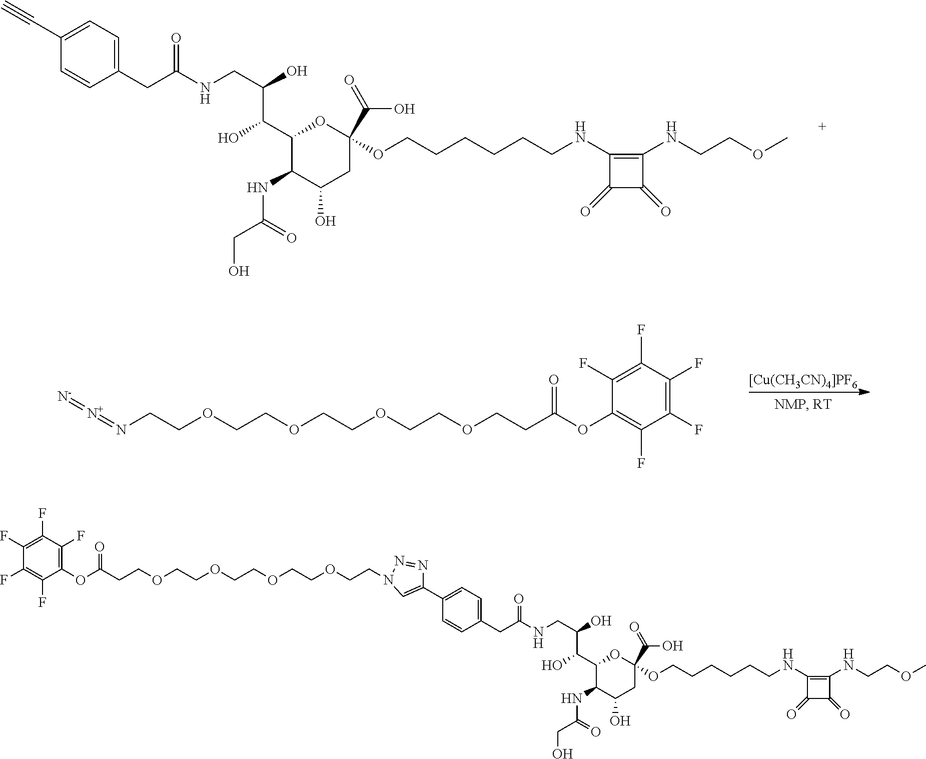

### Synthesis of (2R,4S,5R,6R)-6-((1R,2R)-1,2-dihydroxy-3-(2-(4-(1-(15-oxo-15-(perfluorophenoxy)-3,6,9,12-tetraoxapentadecyl)-1H-1,2,3-triazol-4-yl)phenyl)acetamido)propyl)-4-hydroxy-5-(2-hydroxyacetamido)-2-((6-((2-((2-methoxyethyl)amino)-3,4-dioxocyclobut-1-en-1-yl)amino)hexyl)oxy)tetrahydro-2H-pyran-2-carboxylic acid (Cpd. 36)

To a stirred solution of (2R,4S,5R,6R)-6-((1R,2R)-3-(2-(4-ethynylphenyl)acetamido)-1,2-dihydroxypropyl)-4-hydroxy-5-(2-hydroxyacetamido)-2-((6-((2-((2-methoxyethyl)amino)-3,4-dioxocyclobut-1-en-1-yl)amino)hexyl)oxy)tetrahydro-2H-pyran-2-carboxylic acid (6.16 mg, 8.57 umol) in anhydrous NMP (114 uL) was added Azido-PEG4-PFP-ester (4.31 mg, 9.43 umol), followed by Tetrakis(acetonitrile)copper(l) hexafluorophosphate (8.00 mg, 21.4 umol). The vial was sealed, and the mixture was stirred for 1 h at room temperature. The mixture was filtered through a 0.45 μm PTFE filter. A few drops of acetic acid were added to the filtrate. The filtrate was purified by preparatory HPLC using acetonitrile and water (+0.05% TFA) as eluents. The fractions containing the desired product were lyophilized to afford Cpd. 36 as a white solid. Yield: 4.97 mg, 49%; LCMS (ESI) m/z 1176.40 [M+H]⁺. ¹H NMR (500 MHz, DMSO-d₆): 8.46 (s, 1H), 7.96 (br, 1H), 7.79 (br, 1H), 7.73 (d, J=8.0 Hz, 2H), 7.32 (d, J=8.0 Hz, 2H), 5.51 (t, J=5.5 Hz, 1H), 4.88 (br, 1H), 4.70 (d, J=4.5 Hz, 1H), 4.55 (t, J=5.5 Hz, 2H), 3.91-3.83 (m, 4H), 3.81-3.76 (m, 1H), 3.73 (t, J=6.0 Hz, 2H), 3.70-3.63 (m, 3H), 3.61-3.58 (m, 1H), 3.58-3.52 (m, 5H), 3.52-3.45 (m, 16H), 3.29-3.27 (m, 1H), 3.27 (s, 3H), 3.19-3.15 (m, 1H), 3.00 (t, J=6.0 Hz, 2H), 2.93-2.88 (m, 1H), 2.52-2.50 (m, 1H), 1.54-1.35 (m, 5H), 1.32-1.19 (m, 4H).

### Compound 37

### (2R,4S,5R,6R)-6-((1R,2R)-1,2-dihydroxy-3-(2-(4-(1-(15-oxo-15-(perfluorophenoxy)-3,6,9,12-tetraoxapentadecyl)-1H-1,2,3-triazol-4-yl)phenyl)acetamido)propyl)-4-hydroxy-5-(2-hydroxyacetamido)-2-((6-pentanamidohexyl)oxy)tetrahydro-2H-pyran-2-carboxylic acid (Cpd. 37)

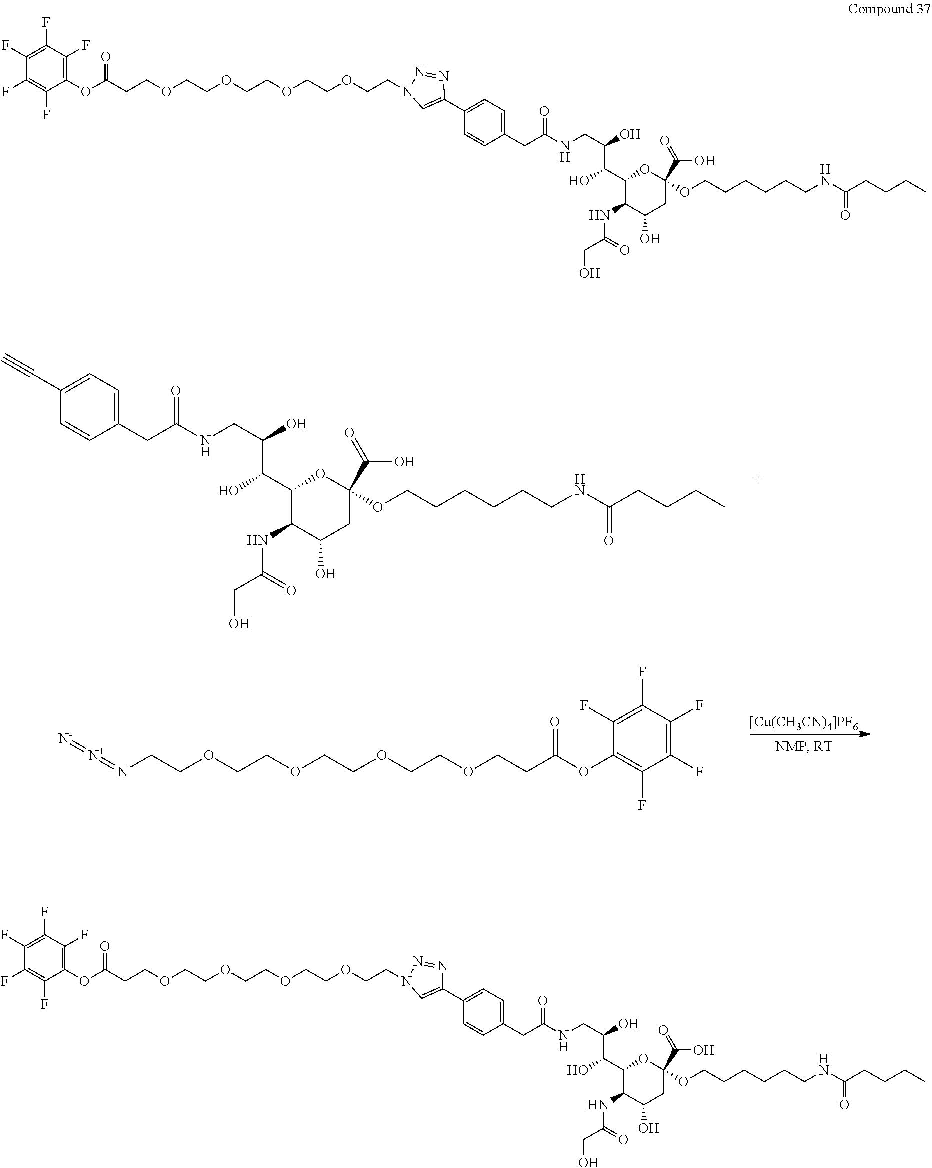

### Synthesis of (2R,4S,5R,6R)-6-((1R,2R)-1,2-dihydroxy-3-(2-(4-(1-(15-oxo-15-(perfluorophenoxy)-3,6,9,12-tetraoxapentadecyl)-1H-1,2,3-triazol-4-yl)phenyl)acetamido)propyl)-4-hydroxy-5-(2-hydroxyacetamido)-2-((6-pentanamidohexyl)oxy)tetrahydro-2H-pyran-2-carboxylic acid (Cpd. 37)

To a stirred solution of (2R,4S,5R,6R)-6-((1R,2R)-3-(2-(4-ethynylphenyl)acetamido)-1,2-dihydroxypropyl)-4-hydroxy-5-(2-hydroxyacetamido)-2-((6-pentanamidohexyl)oxy)tetrahydro-2H-pyran-2-carboxylic acid (19.7 mg, 30.4 umol) in anhydrous NMP (405 uL) was added Azido-PEG4-PFP-ester (15.3 mg, 33.4 umol), followed by Tetrakis(acetonitrile)copper(l) hexafluorophosphate (28.3 mg, 75.9 umol). The vial was sealed, and the mixture was stirred for 1 h at room temperature. The mixture was filtered through a 0.45 μm PTFE filter. A few drops of acetic acid were added to the filtrate. The filtrate was purified by preparatory HPLC using acetonitrile and water (+0.05% TFA) as eluents. The fractions containing the desired product were lyophilized to afford Cpd. 37 as a white solid. Yield: 20.4 mg, 61%; LCMS (ESI) m/z 1107.40 [M+H]⁺. ¹H NMR (500 MHz, DMSO-d6): 8.46 (s, 1H), 7.98 (t, J=5.5 Hz, 1H), 7.84 (d, J=7.5 Hz, 1H), 7.73 (d, J=8.0 Hz, 2H), 7.70 (t, J=5.5 Hz, 1H), 7.31 (d, J=8.0 Hz, 2H), 5.52 (t, J=5.5 Hz, 1H), 4.95 (d, J=5.5 Hz, 1H), 4.71 (d, J=5.5 Hz, 1H), 4.55 (t, J=5.5 Hz, 2H), 4.30 (br, 1H), 3.91-3.83 (m, 4H), 3.81-3.76 (m, 1H), 3.73 (t, J=6.0 Hz, 2H), 3.70-3.66 (m, 1H), 3.65-3.60 (m, 1H), 3.58-3.53 (m, 5H), 3.52-3.45 (m, 13H), 3.29-3.27 (m, 1H), 3.22-3.20 (m, 1H), 3.00 (t, J=6.0 Hz, 2H), 3.00-2.97 (m, 2H), 2.97-2.93 (m, 1H), 2.52-2.50 (m, 1H), 2.03 (t, J=7.5 Hz, 2H), 1.51 (t, J=12.0 Hz, 1H), 1.48-1.42 (m, 4H), 1.37-1.31 (m, 2H), 1.27-1.19 (m, 6H), 0.84 (t, J=7.5 Hz, 3H).

### Compound 38

### (2R,4S,5R,6R)-2-((5-carboxypentyl)oxy)-6-((1R,2R)-3-(2-(4-chlorophenyl)acetamido)-1,2-dihydroxypropyl)-5-(2-hydroxyacetamido)-4-((1-(15-oxo-15-(perfluorophenoxy)-3,6,9,12-tetraoxapentadecyl)-1H-1,2,3-triazol-4-yl)methoxy)tetrahydro-2H-pyran-2-carboxylic acid (Cpd. 38)

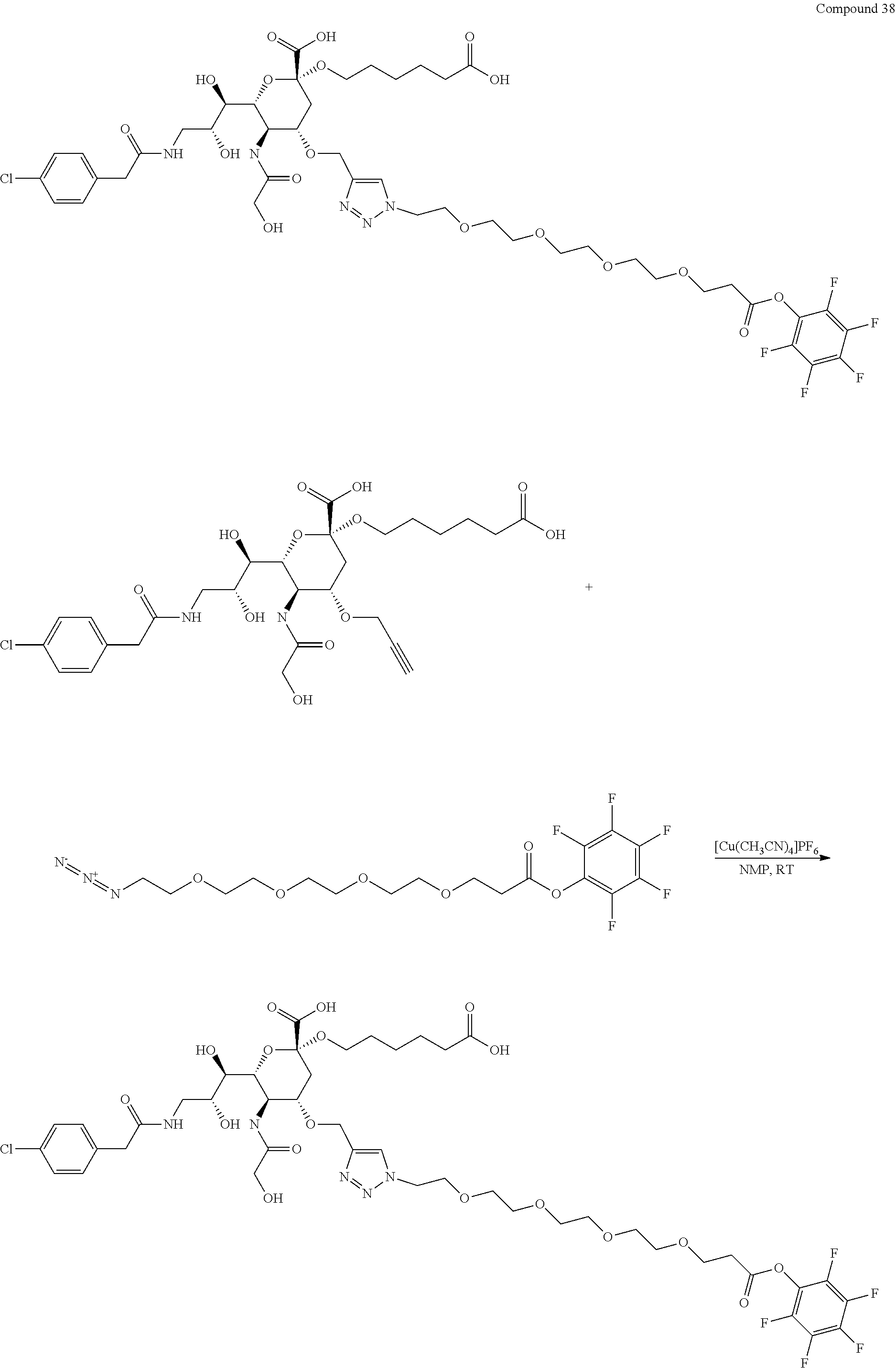

### Synthesis of (2R,4S,5R,6R)-2-((5-carboxypentyl)oxy)-6-((1R,2R)-3-(2-(4-chlorophenyl)acetamido)-1,2-dihydroxypropyl)-5-(2-hydroxyacetamido)-4-((1-(15-oxo-15-(perfluorophenoxy)-3,6,9,12-tetraoxapentadecyl)-1H-1,2,3-triazol-4-yl)methoxy)tetrahydro-2H-pyran-2-carboxylic acid (Cpd. 38)

To a stirred solution of (2R,4S,5R,6R)-2-((5-carboxypentyl)oxy)-6-((1R,2R)-3-(2-(4-chlorophenyl)acetamido)-1,2-dihydroxypropyl)-5-(2-hydroxyacetamido)-4-(prop-2-yn-1-yloxy)tetrahydro-2H-pyran-2-carboxylic acid (20.9 mg, 33.3 umol) in anhydrous NMP (444 uL) was added Azido-PEG4-PFP-ester (16.7 mg, 36.6 umol), followed by Tetrakis(acetonitrile)copper(l) hexafluorophosphate (31.0 mg, 83.2 umol). The vial was sealed, and the mixture was stirred for 1 h at room temperature. The mixture was filtered through a 0.45 μm PTFE filter. A few drops of acetic acid were added to the filtrate. The filtrate was purified by preparatory HPLC using acetonitrile and water (+0.05% TFA) as eluents. The fractions containing the desired product were lyophilized to afford Cpd. 38 as a white solid. Yield: 21.2 mg, 59%; LCMS (ESI) m/z 1086.30 [M+H]⁺. ¹H NMR (500 MHz, DMSO-d₆): 11.98 (br, 1H), 7.99 (d, J=7.5 Hz, 1H), 7.96 (t, J=5.5 Hz, 1H), 7.95 (s, 1H), 7.33 (d, J=8.5 Hz, 2H), 7.27 (d, J=8.5 Hz, 2H), 5.58 (t, J=5.5 Hz, 1H), 4.61 (d, J=5.5 Hz, 1H), 4.60 (d, J=12.0 Hz, 1H), 4.54 (d, J=12.0 Hz, 1H), 4.49 (t, J=5.5 Hz, 2H), 3.94-3.88 (m, 1H), 3.87-3.84 (m, 2H), 3.80 (t, J=6.0 Hz, 2H), 3.76 (t, J=6.0 Hz, 2H), 3.72-3.68 (m, 1H), 3.67-3.59 (m, 3H), 3.56-3.46 (m, 14H), 3.44 (s, 2H), 3.21-3.18 (m, 1H), 3.02 (t, J=7.5 Hz, 2H), 2.97-2.91 (m, 1H), 2.70-2.65 (m, 1H), 2.18 (t, J=7.5 Hz, 2H), 1.50-1.42 (m, 5H), 1.29-1.23 (m, 2H).

### Compound 39

### (2R,4S,5R,6R)-2-(benzyloxy)-6-((1R,2R)-3-(2-(4-chlorophenyl)acetamido)-1,2-dihydroxypropyl)-5-(2-hydroxyacetamido)-4-((1-(15-oxo-15-(perfluorophenoxy)-3,6,9,12-tetraoxapentadecyl)-1H-1,2,3-triazol-4-yl)methoxy)tetrahydro-2H-pyran-2-carboxylic acid (Cpd. 39)

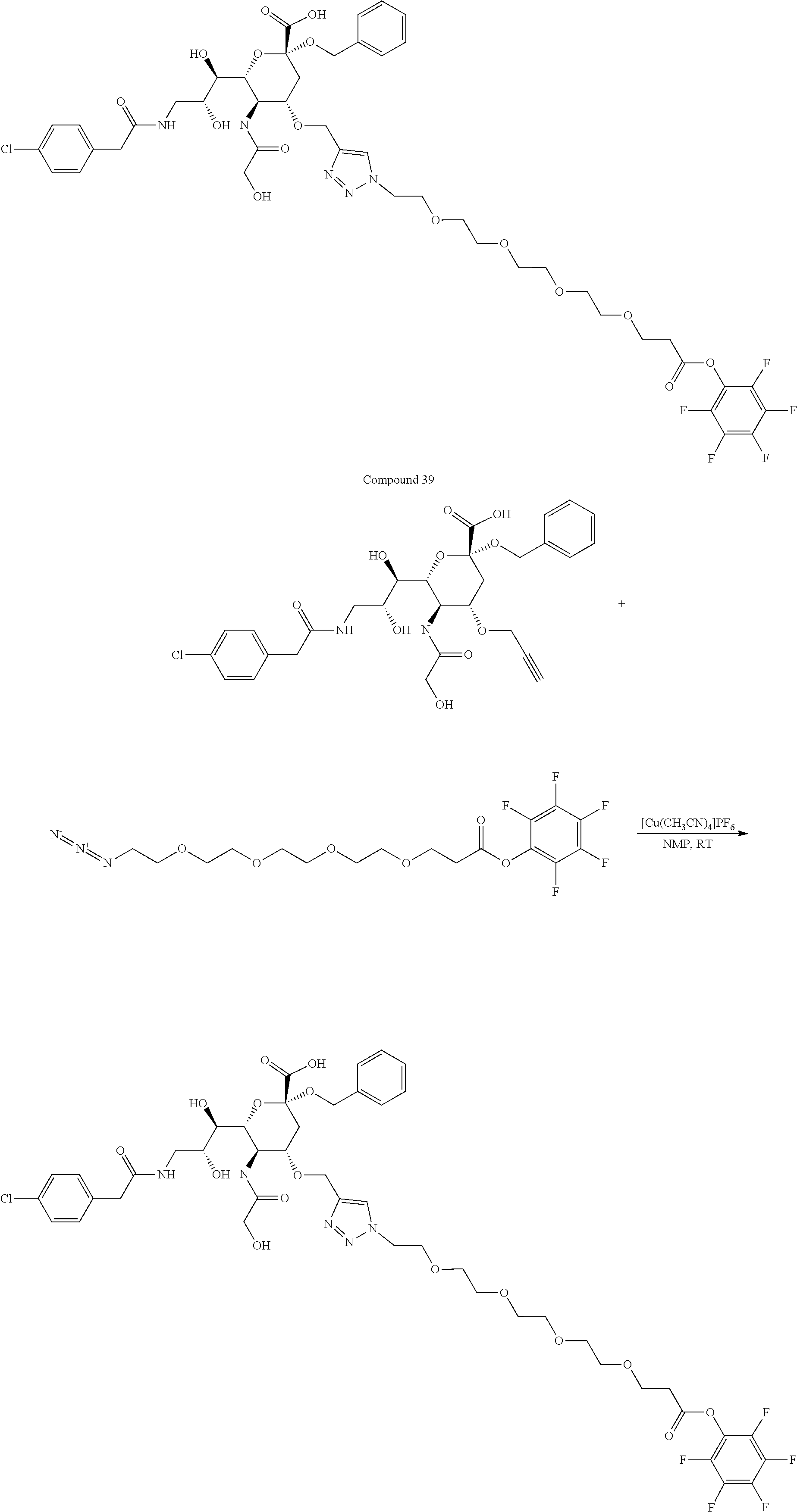

### Synthesis of (2R,4S,5R,6R)-2-(benzyloxy)-6-((1R,2R)-3-(2-(4-chlorophenyl)acetamido)-1,2-dihydroxypropyl)-5-(2-hydroxyacetamido)-4-((1-(15-oxo-15-(perfluorophenoxy)-3,6,9,12-tetraoxapentadecyl)-1H-1,2,3-triazol-4-yl)methoxy)tetrahydro-2H-pyran-2-carboxylic acid (Cpd. 39)

To a stirred solution of (2R,4S,5R,6R)-2-(benzyloxy)-6-((1R,2R)-3-(2-(4-chlorophenyl)acetamido)-1,2-dihydroxypropyl)-5-(2-hydroxyacetamido)-4-(prop-2-yn-1-yloxy)tetrahydro-2H-pyran-2-carboxylic acid (20.4 mg, 33.7 umol) in anhydrous NMP (449 uL) was added Azido-PEG4-PFP-ester (17.0 mg, 37.1 umol), followed by Tetrakis(acetonitrile)copper(l) hexafluorophosphate (31.4 mg, 84.3 umol). The vial was sealed, and the mixture was stirred for 1 h at room temperature. The mixture was filtered through a 0.45 μm PTFE filter. A few drops of acetic acid were added to the filtrate. The filtrate was purified by preparatory HPLC using acetonitrile and water (+0.05% TFA) as eluents. The fractions containing the desired product were lyophilized to afford Cpd. 39 as a white solid. Yield: 14.9 mg, 42%; LCMS (ESI) m/z 1062.30 [M+H]⁺. ¹H NMR (500 MHz, DMSO-d₆): 8.02 (d, J=8.0 Hz, 1H), 7.97 (t, J=5.5 Hz, 1H), 7.95 (s, 1H), 7.35-7.25 (m, 9H), 5.59 (t, J=5.5 Hz, 1H), 4.72 (d, J=12.0 Hz, 1H), 4.64 (d, J=5.5 Hz, 1H), 4.62 (d, J=12.0 Hz, 1H), 4.57 (d, J=12.0 Hz, 1H), 4.49 (t, J=5.5 Hz, 2H), 4.46 (d, J=12.0 Hz, 1H), 4.00-3.95 (m, 1H), 3.91-3.83 (m, 2H), 3.80 (t, J=6.0 Hz, 2H), 3.78-3.70 (m, 3H), 3.75 (t, J=6.0 Hz, 2H), 3.55-3.45 (m, 13H), 3.44 (s, 2H), 3.26-3.23 (m, 1H), 3.01 (t, J=6.0 Hz, 2H), 2.99-2.95 (m, 1H), 2.74-2.71 (m, 1H), 1.57 (t, J=12.0 Hz, 1H).

### Compound 40

### (2R,4S,5R,6R)-2-(benzyloxy)-6-((1R,2R)-3-(2-(4-chlorophenyl)acetamido)-1,2-dihydroxypropyl)-4-hydroxy-5-(3-(1-(1-(15-oxo-15-(perfluorophenoxy)-3,6,9,12-tetraoxapentadecyl)-1H-1,2,3-triazol-4-yl)-2,5,8,11,14-pentaoxahexadecan-16-yl)ureido)tetrahydro-2H-pyran-2-carboxylic acid (Cpd. 40)

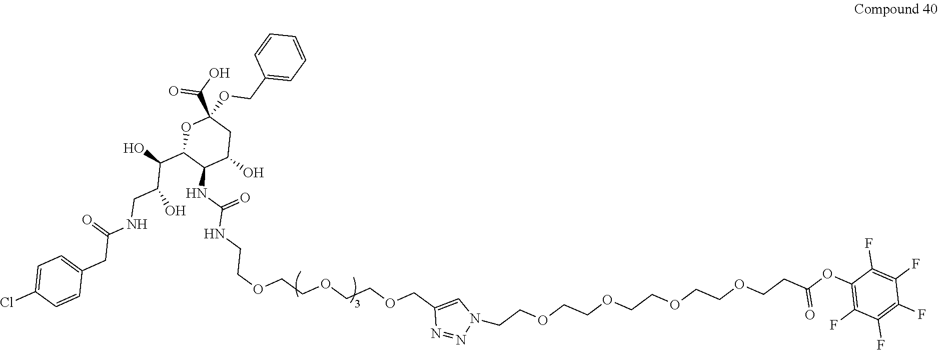

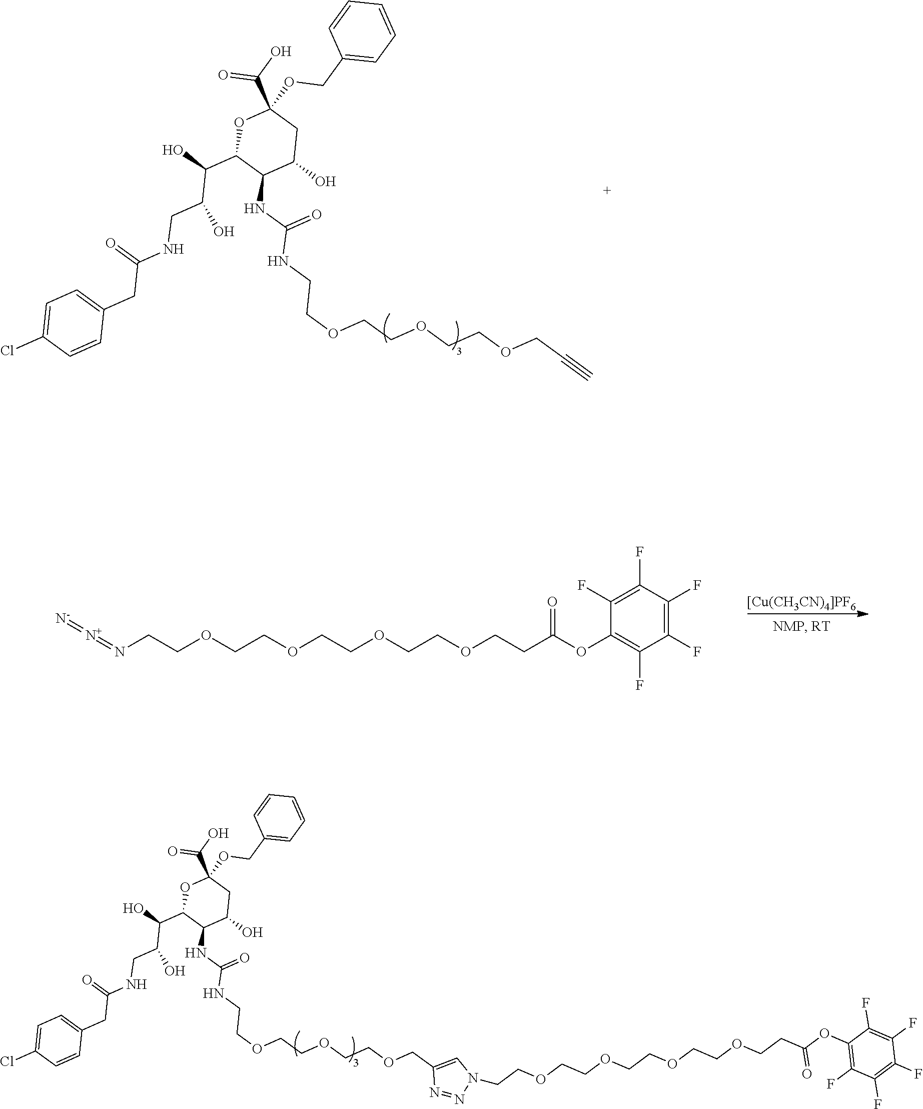

### Synthesis of (2R,4S,5R,6R)-2-(benzyloxy)-6-((1R,2R)-3-(2-(4-chlorophenyl)acetamido)-1,2-dihydroxypropyl)-4-hydroxy-5-(3-(1-(1-(15-oxo-15-(perfluorophenoxy)-3,6,9,12-tetraoxapentadecyl)-1H-1,2,3-triazol-4-yl)-2,5,8,11,14-pentaoxahexadecan-16-yl)ureido)tetrahydro-2H-pyran-2-carboxylic acid (Cpd. 40)

To a stirred solution of (2R,4S,5R,6R)-5-(3-(3,6,9,12,15-pentaoxaoctadec-17-yn-1-yl)ureido)-2-(benzyloxy)-6-((1R,2R)-3-(2-(4-chlorophenyl)acetamido)-1,2-dihydroxypropyl)-4-hydroxytetrahydro-2H-pyran-2-carboxylic acid (8.70 mg, 10.7 umol) in anhydrous NMP (143 uL) was added Azido-PEG4-PFP-ester (5.40 mg, 11.8 umol), followed by Tetrakis(acetonitrile)copper(l) hexafluorophosphate (10.0 mg, 26.8 umol). The vial was sealed, and the mixture was stirred for 1 h at room temperature. The mixture was filtered through a 0.45 μm PTFE filter. A few drops of acetic acid were added to the filtrate. The filtrate was purified by preparatory HPLC using acetonitrile and water (+0.05% TFA) as eluents. The fractions containing the desired product were lyophilized to afford Cpd. 40 as a white solid. Yield: 7.40 mg, 54%; LCMS (ESI) m/z 1267.50 [M+H]⁺. ¹H NMR (500 MHz, DMSO-d₆): 8.04 (s, 1H), 7.96 (t, J=5.5 Hz, 1H), 7.35-7.25 (m, 9H), 6.21-6.19 (m, 2H), 5.37 (d, J=5.0 Hz, 1H), 5.07 (br, 1H), 4.70 (d, J=12.0 Hz, 1H), 4.51 (s, 2H), 4.50 (t, J=5.5 Hz, 2H), 4.43 (d, J=12.0 Hz, 1H), 3.80 (t, J=5.5 Hz, 2H), 3.76 (t, J=5.5 Hz, 2H), 3.74-3.71 (m, 1H), 3.59-3.56 (m, 1H), 3.55-3.53 (m, 4H), 3.52-3.45 (m, 25H), 3.44 (s, 2H), 3.39 (t, J=5.5 Hz, 2H), 3.37-3.36 (m, 2H), 3.29-3.27 (m, 1H), 3.19-3.16 (m, 2H), 3.01 (t, J=6.0 Hz, 2H), 2.97-2.92 (m, 1H), 2.61-2.58 (m, 1H), 1.59 (t, J=12.0 Hz, 1H).

### Compound 41

### (2S,4S,5R,6R)-2-(benzylthio)-6-((1R,2R)-1,2-dihydroxy-3-(2-(4-(1-(37-oxo-41-((3aS,4S,6aR)-2-oxohexahydro-1H-thieno[3,4-d]imidazol-4-yl)-3,6,9,12,15,18,21,24,27,30,33-undecaoxa-36-azahentetracontyl)-1H-1,2,3-triazol-4-yl)phenyl)acetamido)propyl)-4-hydroxy-5-(2-hydroxyacetamido)tetrahydro-2H-pyran-2-carboxylic acid (Cpd. 41)

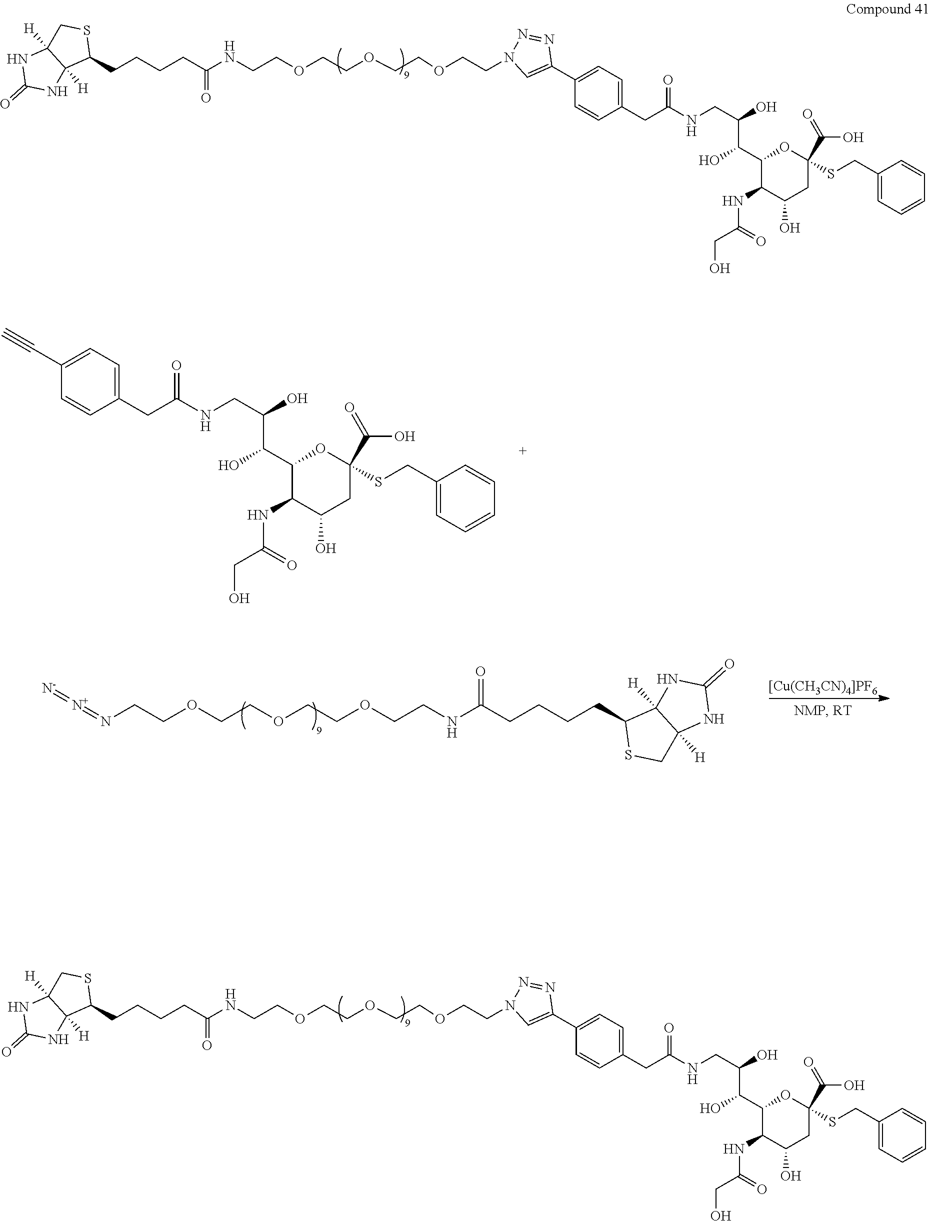

### Synthesis of (2S,4S,5R,6R)-2-(benzylthio)-6-((1R,2R)-1,2-dihydroxy-3-(2-(4-(1-(37-oxo-41-((3aS,4S,6aR)-2-oxohexahydro-1H-thieno[3,4-d]imidazol-4-yl)-3,6,9,12,15,18,21,24,27,30,33-undecaoxa-36-azahentetracontyl)-1H-1,2,3-triazol-4-yl)phenyl)acetamido)propyl)-4-hydroxy-5-(2-hydroxyacetamido)tetrahydro-2H-pyran-2-carboxylic acid (Cpd. 41)

To a stirred solution of (2S,4S,5R,6R)-2-(benzylthio)-6-((1R,2R)-3-(2-(4-ethynylphenyl)acetamido)-1,2-dihydroxypropyl)-4-hydroxy-5-(2-hydroxyacetamido)tetrahydro-2H-pyran-2-carboxylic acid (12.2 mg, 21.2 umol) in anhydrous NMP (350 uL) was added Azido-PEG11-Biotin (20.3 mg, 25.5 umol), followed by Tetrakis(acetonitrile)copper(l) hexafluorophosphate (11.9 mg, 31.9 umol). The vial was sealed, and the mixture was stirred for 1 h at room temperature. The mixture was filtered through a 0.45 μm PTFE filter. A few drops of acetic acid were added to the filtrate. The filtrate was purified by preparatory HPLC using acetonitrile and water (+0.05% TFA) as eluents. The fractions containing the desired product were lyophilized to afford Cpd. 41 as a white solid. Yield: 22.8 mg, 78%; LCMS (ESI) m/z 1369.60 [M+H]⁺. ¹H NMR (500 MHz, DMSO-d6): 8.42 (s, 1H), 7.95 (t, J=5.5 Hz, 1H), 7.87 (d, J=8.0 Hz, 1H), 7.82 (t, J=5.5 Hz, 1H), 7.71 (d, J=8.5 Hz, 2H), 7.31 (d, J=8.5 Hz, 2H), 7.29-7.25 (m, 4H), 7.22-7.19 (m, 1H), 6.40 (s, 1H), 6.34 (s, 1H), 5.53 (t, J=5.5 Hz, 1H), 5.00 (br, 1H), 4.76 (d, J=5.0 Hz, 1H), 4.55 (t, J=5.0 Hz, 2H), 4.31-4.28 (m, 1H), 4.13-4.10 (m, 1H), 3.95 (d, J=12.5 Hz, 1H), 3.91-3.83 (m, 6H), 3.73-3.69 (m, 1H), 3.61-3.58 (m, 1H), 3.56-3.54 (m, 4H), 3.51-3.49 (m, 28H), 3.48-3.46 (m, 12H), 3.38 (t, J=6.0 Hz, 2H), 3.22-3.18 (m, 1H), 3.19-3.16 (m, 2H), 3.11-3.07 (m, 1H), 2.95-2.89 (m, 1H), 2.81 (dd, J=12.5 Hz, 5.0 Hz, 1H), 2.63-2.58 (m, 1H), 2.57 (d, J=12.5 Hz, 1H), 2.06 (t, J=7.5 Hz, 2H), 1.64-1.53 (m, 2H), 1.53-1.41 (m, 3H), 1.33-1.23 (m, 2H).

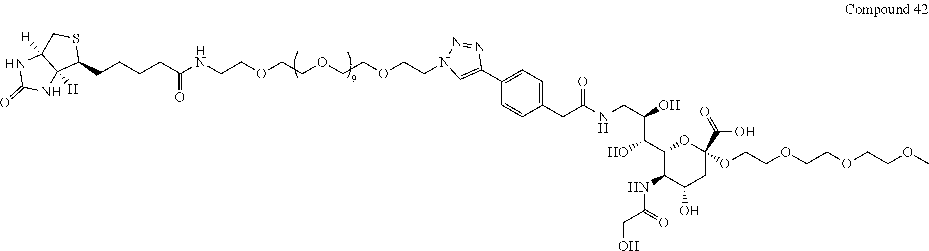

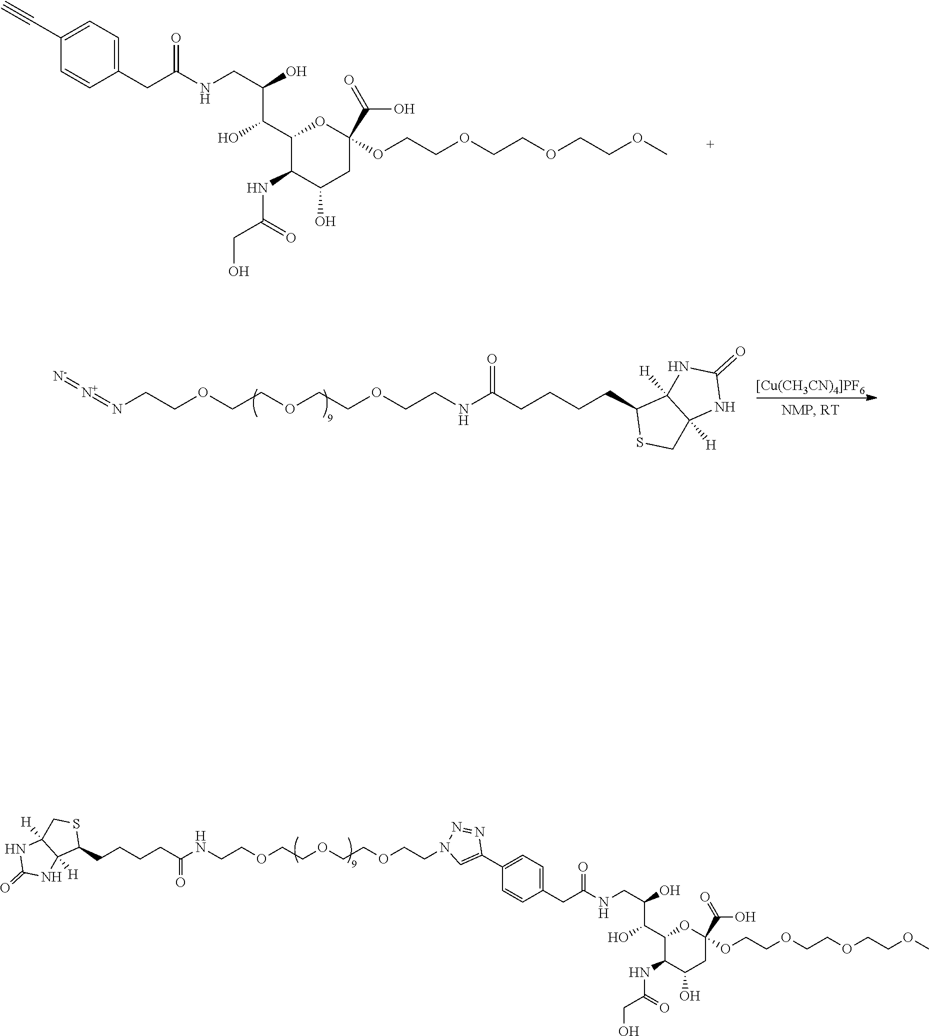

### Synthesis of (2R,4S,5R,6R)-6-((1R,2R)-1,2-dihydroxy-3-(2-(4-(1-(37-oxo-41-((3aS,4S,6aR)-2-oxohexahydro-1H-thieno[3,4-d]imidazol-4-yl)-3,6,9,12,15,18,21,24,27,30,33-undecaoxa-36-azahentetracontyl)-1H-1,2,3-triazol-4-yl)phenyl)acetamido)propyl)-4-hydroxy-5-(2-hydroxyacetamido)-2-(2-(2-(2-methoxyethoxy)ethoxy)ethoxy)tetrahydro-2H-pyran-2-carboxylic acid (Cpd. 42)

To a stirred solution of (2R,4S,5R,6R)-6-((1R,2R)-3-(2-(4-ethynylphenyl)acetamido)-1,2-dihydroxypropyl)-4-hydroxy-5-(2-hydroxyacetamido)-2-(2-(2-(2-methoxyethoxy)ethoxy)ethoxy) tetrahydro-2H-pyran-2-carboxylic acid (8.40 mg, 13.7 umol) in anhydrous NMP (183 uL) was added Azido-PEG11-Biotin (13.1 mg, 16.5 umol), followed by Tetrakis(acetonitrile)copper(l) hexafluorophosphate (7.67 mg, 20.6 umol). The vial was sealed, and the mixture was stirred for 1 h at room temperature. The mixture was filtered through a 0.45 μm PTFE filter. A few drops of acetic acid were added to the filtrate. The filtrate was purified by preparatory HPLC using acetonitrile and water (+0.05% TFA) as eluents. The fractions containing the desired product were lyophilized to afford Cpd. 42 as a white solid. Yield: 13.8 mg, 71%; LCMS (ESI) m/z 1409.70 [M+H]⁺. ¹H NMR (500 MHz, DMSO-d₆): 8.47 (s, 1H), 7.96 (t, J=5.5 Hz, 1H), 7.82 (d, J=5.5 Hz, 1H), 7.82 (t, J=5.5 Hz, 1H), 7.74 (d, J=8.5 Hz, 2H), 7.32 (d, J=8.5 Hz, 2H), 6.40 (s, 1H), 6.34 (s, 1H), 5.52 (t, J=5.5 Hz, 1H), 4.94 (br, 1H), 4.71 (d, J=5.0 Hz, 1H), 4.56 (t, J=5.0 Hz, 2H), 4.31-4.28 (m, 1H), 4.13-4.11 (m, 1H), 3.92-3.83 (m, 4H), 3.81-3.78 (m, 1H), 3.77-3.73 (m, 1H), 3.70-3.66 (m, 1H), 3.56-3.54 (m, 4H), 3.51-3.45 (m, 51H), 3.44-3.41 (m, 2H), 3.38 (t, J=6.0 Hz, 2H), 3.23 (s, 3H), 3.21-3.17 (m, 1H), 3.19-3.16 (m, 2H), 3.11-3.07 (m, 1H), 2.97-2.92 (m, 1H), 2.81 (dd, J=12.5 Hz, 5.0 Hz, 1H), 2.57 (d, J=12.5 Hz, 1H), 2.06 (t, J=7.5 Hz, 2H), 1.64-1.57 (m, 1H), 1.53-1.42 (m, 4H), 1.33-1.23 (m, 2H).

### Compound 43

### (2R,4S,5R,6R)-6-((1R,2R)-1,2-dihydroxy-3-(2-(4-(1-(37-oxo-41-((3aS,4S,6aR)-2-oxohexahydro-1H-thieno[3,4-d]imidazol-4-yl)-3,6,9,12,15,18,21,24,27,30,33-undecaoxa-36-azahentetracontyl)-1H-1,2,3-triazol-4-yl)phenyl)acetamido)propyl)-2-(hexyloxy)-4-hydroxy-5-(2-hydroxyacetamido)tetrahydro-2H-pyran-2-carboxylic acid (Cpd. 43)

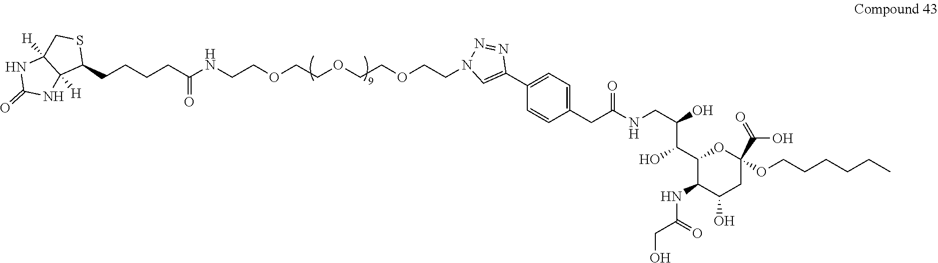

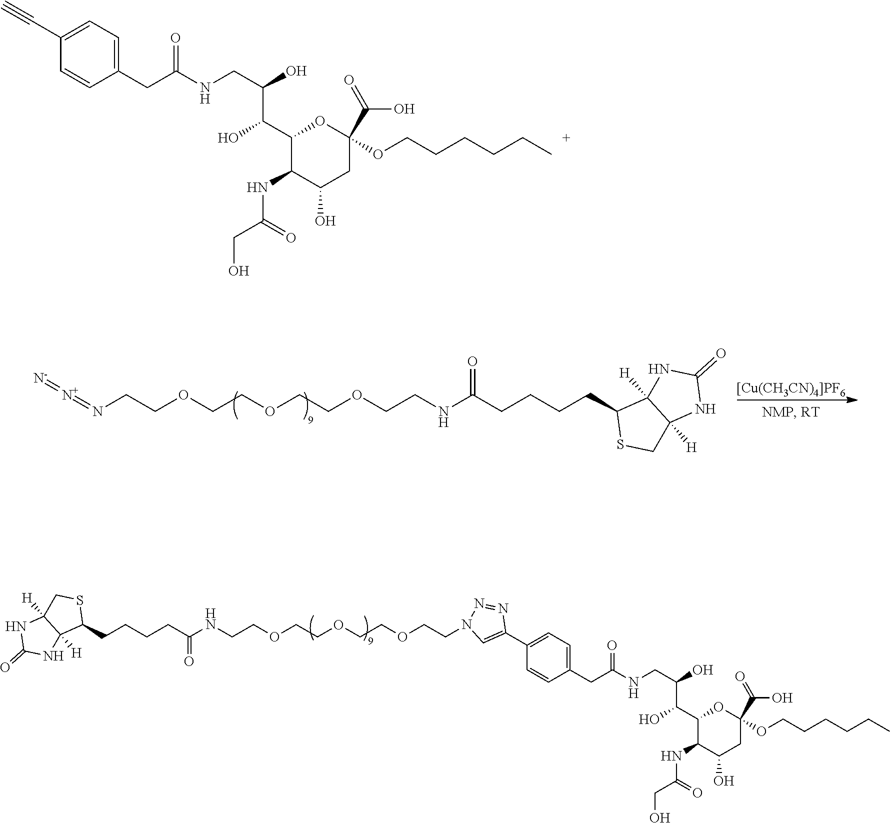

### Synthesis of (2R,4S,5R,6R)-6-((1R,2R)-1,2-dihydroxy-3-(2-(4-(1-(37-oxo-41-((3aS,4S,6aR)-2-oxohexahydro-1H-thieno[3,4-d]imidazol-4-yl)-3,6,9,12,15,18,21,24,27,30,33-undecaoxa-36-azahentetracontyl)-1H-1,2,3-triazol-4-yl)phenyl)acetamido)propyl)-2-(hexyloxy)-4-hydroxy-5-(2-hydroxyacetamido)tetrahydro-2H-pyran-2-carboxylic acid (Cpd. 43)

To a stirred solution of (2R,4S,5R,6R)-6-((1R,2R)-3-(2-(4-ethynylphenyl)acetamido)-1,2-dihydroxypropyl)-2-(hexyloxy)-4-hydroxy-5-(2-hydroxyacetamido)tetrahydro-2H-pyran-2-carboxylic acid (2.10 mg, 3.81 umol) in anhydrous NMP (51 uL) was added Azido-PEG11-Biotin (3.65 mg, 4.58 umol), followed by Tetrakis(acetonitrile)copper(l) hexafluorophosphate (2.13 mg, 5.72 umol). The vial was sealed, and the mixture was stirred for 1 h at room temperature. The mixture was filtered through a 0.45 μm PTFE filter. A few drops of acetic acid were added to the filtrate. The filtrate was purified by preparatory HPLC using acetonitrile and water (+0.05% TFA) as eluents. The fractions containing the desired product were lyophilized to afford Cpd. 43 as a white solid. Yield: 3.50 mg, 68%; LCMS (ESI) m/z 1347.70 [M+H]⁺. ¹H NMR (500 MHz, DMSO-d₆): 8.47 (s, 1H), 7.95 (t, J=5.5 Hz, 1H), 7.82 (t, J=5.5 Hz, 1H), 7.79 (br, 1H), 7.74 (d, J=8.0 Hz, 2H), 7.32 (d, J=8.0 Hz, 2H), 6.40 (s, 1H), 6.34 (s, 1H), 5.51 (t, J=5.5 Hz, 1H), 4.87 (br, 1H), 4.69 (d, J=5.0 Hz, 1H), 4.55 (t, J=5.0 Hz, 2H), 4.31-4.28 (m, 1H), 4.13-4.11 (m, 1H), 3.92-3.83 (m, 4H), 3.79-3.71 (m, 1H), 3.69-3.65 (m, 1H), 3.63-3.58 (m, 1H), 3.58-3.53 (m, 3H), 3.51-3.45 (m, 43H), 3.38 (t, J=5.5 Hz, 2H), 3.29-3.27 (m, 1H), 3.19-3.16 (m, 3H), 3.11-3.07 (m, 1H), 2.97-2.92 (m, 1H), 2.81 (dd, J=12.5 Hz, 5.0 Hz, 1H), 2.57 (d, J=12.5 Hz, 1H), 2.06 (t, J=7.5 Hz, 2H), 1.64-1.57 (m, 1H), 1.52-1.40 (m, 6H), 1.33-1.21 (m, 8H), 0.84 (t, J=7.0 Hz, 3H).

### Compound 44

### (2R,4S,5R,6R)-6-((1R,2R)-1,2-dihydroxy-3-(2-(4-(1-(37-oxo-41-((3aS,4S,6aR)-2-oxohexahydro-1H-thieno[3,4-d]imidazol-4-yl)-3,6,9,12,15,18,21,24,27,30,33-undecaoxa-36-azahentetracontyl)-1H-1,2,3-triazol-4-yl)phenyl)acetamido)propyl)-4-hydroxy-5-(2-hydroxyacetamido)-2-((6-methoxyhexyl)oxy)tetrahydro-2H-pyran-2-carboxylic acid (Cpd. 44)

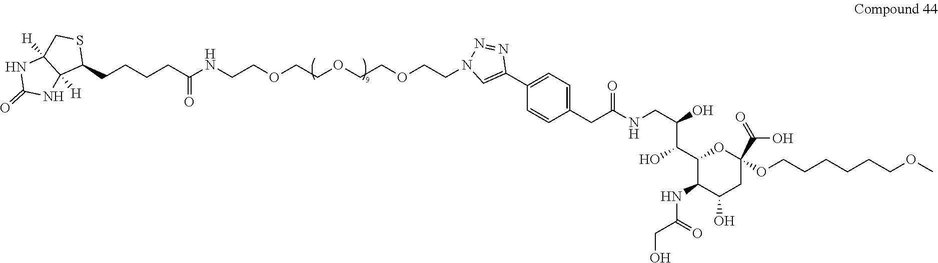

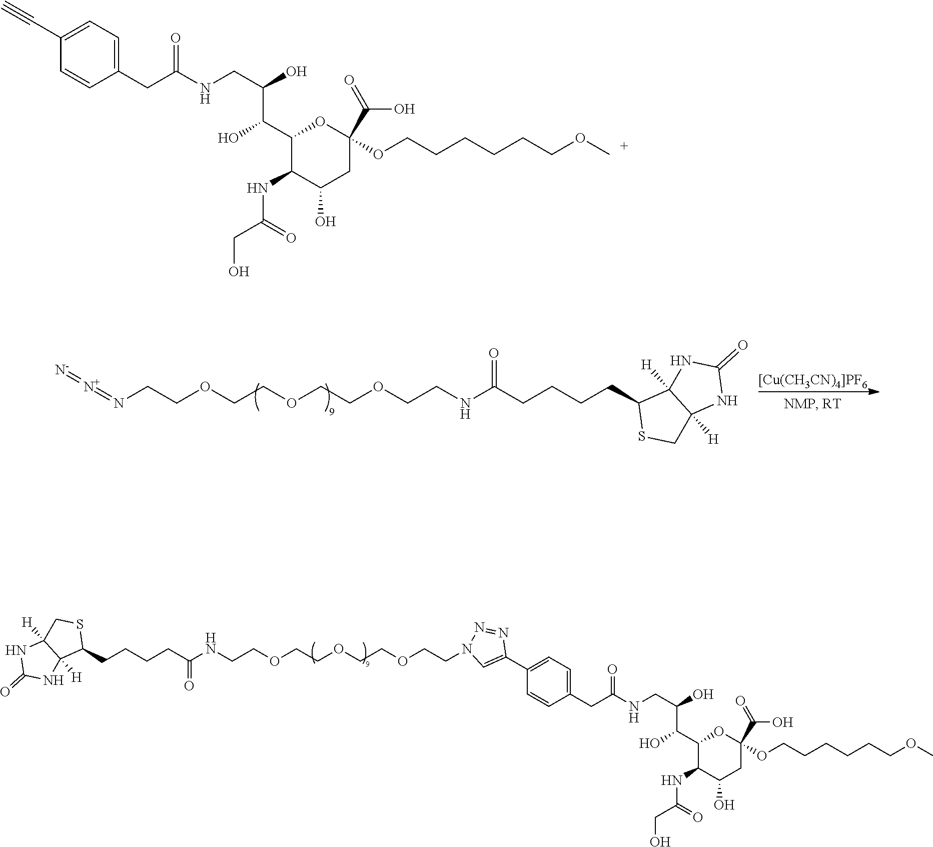

### Synthesis of (2R,4S,5R,6R)-6-((1R,2R)-1,2-dihydroxy-3-(2-(4-(1-(37-oxo-41-((3aS,4S,6aR)-2-oxohexahydro-1H-thieno[3,4-d]imidazol-4-yl)-3,6,9,12,15,18,21,24,27,30,33-undecaoxa-36-azahentetracontyl)-1H-1,2,3-triazol-4-yl)phenyl)acetamido)propyl)-4-hydroxy-5-(2-hydroxyacetamido)-2-((6-methoxyhexyl)oxy)tetrahydro-2H-pyran-2-carboxylic acid (Cpd. 44)

To a stirred solution of (2R,4S,5R,6R)-6-((1R,2R)-3-(2-(4-ethynylphenyl)acetamido)-1,2-dihydroxypropyl)-4-hydroxy-5-(2-hydroxyacetamido)-2-((6-methoxyhexyl)oxy)tetrahydro-2H-pyran-2-carboxylic acid (6.11 mg, 10.5 umol) in anhydrous NMP (140 uL) was added Azido-PEG11-Biotin (10.1 mg, 12.6 umol), followed by Tetrakis(acetonitrile)copper(l) hexafluorophosphate (5.88 mg, 15.8 umol). The vial was sealed, and the mixture was stirred for 1 h at room temperature. The mixture was filtered through a 0.45 μm PTFE filter. A few drops of acetic acid were added to the filtrate. The filtrate was purified by preparatory HPLC using acetonitrile and water (+0.05% TFA) as eluents. The fractions containing the desired product were lyophilized to afford Cpd. 44 as a white solid. Yield: 10.2 mg, 70%; LCMS (ESI) m/z 1377.70 [M+H]⁺. ¹H NMR (500 MHz, DMSO-d6): 8.47 (s, 1H), 7.96 (t, J=5.5 Hz, 1H), 7.83 (d, J=5.5 Hz, 1H), 7.82 (t, J=5.5 Hz, 1H), 7.74 (d, J=8.0 Hz, 2H), 7.32 (d, J=8.0 Hz, 2H), 6.40 (s, 1H), 6.34 (s, 1H), 5.52 (t, J=5.5 Hz, 1H), 4.93 (br, 1H), 4.70 (d, J=5.0 Hz, 1H), 4.56 (t, J=5.0 Hz, 2H), 4.31-4.29 (m, 1H), 4.13-4.11 (m, 1H), 3.91-3.83 (m, 4H), 3.81-3.76 (m, 1H), 3.69-3.65 (m, 1H), 3.64-3.59 (m, 1H), 3.57-3.53 (m, 4H), 3.50-3.46 (m, 42H), 3.39 (t, J=6.0 Hz, 2H), 3.29-3.27 (m, 1H), 3.27 (t, J=6.5 Hz, 2H), 3.21-3.17 (m, 1H), 3.19 (s, 3H), 3.19-3.16 (m, 2H), 3.11-3.07 (m, 1H), 2.97-2.90 (m, 1H), 2.81 (dd, J=12.5 Hz, 5.0 Hz, 1H), 2.57 (d, J=12.5 Hz, 1H), 2.06 (t, J=7.5 Hz, 2H), 1.63-1.57 (m, 1H), 1.52-1.41 (m, 8H), 1.31-1.23 (m, 6H).

### Compound 45

### (2R,4S,5R,6R)-6-((1R,2R)-1,2-dihydroxy-3-(2-(4-(1-(37-oxo-41-((3aS,4S,6aR)-2-oxohexahydro-1H-thieno[3,4-d]imidazol-4-yl)-3,6,9,12,15,18,21,24,27,30,33-undecaoxa-36-azahentetracontyl)-1H-1,2,3-triazol-4-yl)phenyl)acetamido)propyl)-4-hydroxy-5-(2-hydroxyacetamido)-2-((6-((2-((2-methoxyethyl)amino)-3,4-dioxocyclobut-1-en-1-yl)amino)hexyl)oxy)tetrahydro-2H-pyran-2-carboxylic acid (Cpd. 45)

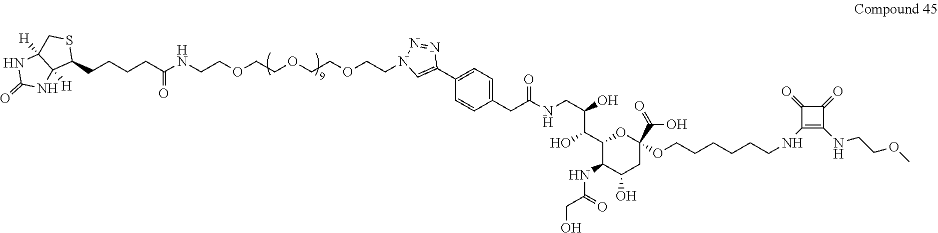

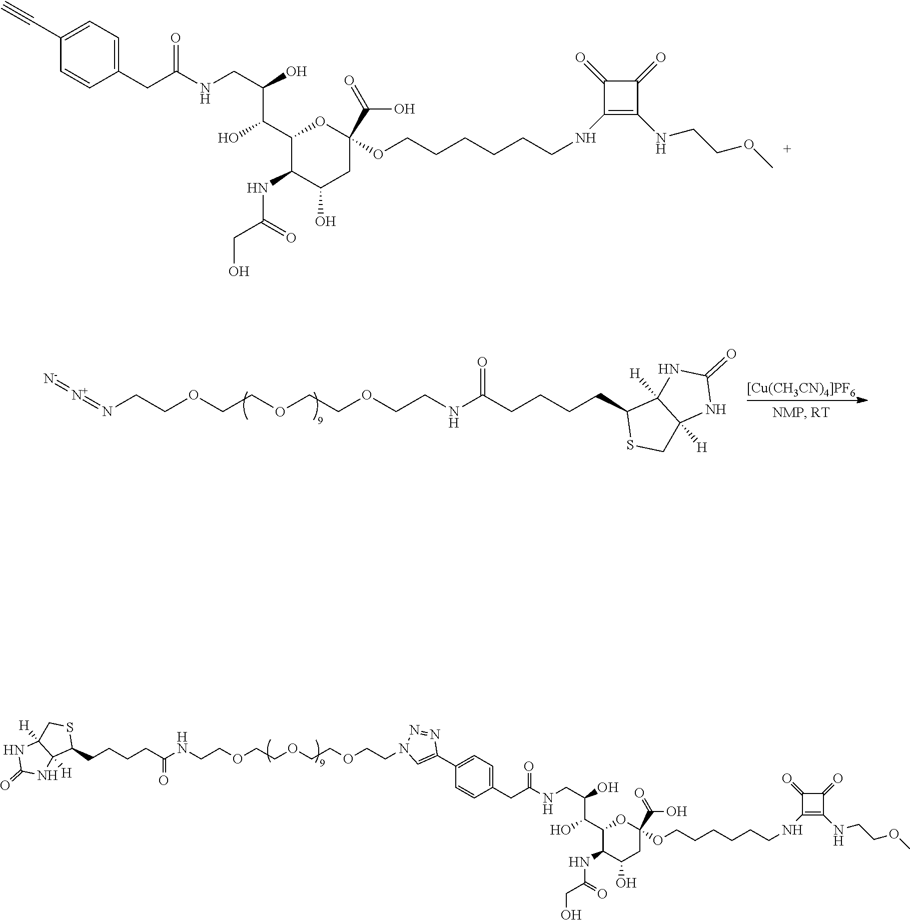

### Synthesis of (2R,4S,5R,6R)-6-((1R,2R)-1,2-dihydroxy-3-(2-(4-(1-(37-oxo-41-((3aS,4S,6aR)-2-oxohexahydro-1H-thieno[3,4-d]imidazol-4-yl)-3,6,9,12,15,18,21,24,27,30,33-undecaoxa-36-azahentetracontyl)-1H-1,2,3-triazol-4-yl)phenyl)acetamido)propyl)-4-hydroxy-5-(2-hydroxyacetamido)-2-((6-((2-((2-methoxyethyl)amino)-3,4-dioxocyclobut-1-en-1-yl)amino)hexyl)oxy)tetrahydro-2H-pyran-2-carboxylic acid (Cpd. 45)

To a stirred solution of (2R,4S,5R,6R)-6-((1R,2R)-3-(2-(4-ethynylphenyl)acetamido)-1,2-dihydroxypropyl)-4-hydroxy-5-(2-hydroxyacetamido)-2-((6-((2-((2-methoxyethyl)amino)-3,4-dioxocyclobut-1-en-1-yl)amino)hexyl)oxy)tetrahydro-2H-pyran-2-carboxylic acid (1.80 mg, 2.50 umol) in anhydrous NMP (35 uL) was added Azido-PEG11-Biotin (2.40 mg, 3.01 umol), followed by Tetrakis(acetonitrile)copper(l) hexafluorophosphate (1.40 mg, 3.76 umol). The vial was sealed, and the mixture was stirred for 1 h at room temperature. The mixture was filtered through a 0.45 μm PTFE filter. A few drops of acetic acid were added to the filtrate. The filtrate was purified by preparatory HPLC using acetonitrile and water (+0.05% TFA) as eluents. The fractions containing the desired product were lyophilized to afford Cpd. 45 as a white solid. Yield: 1.63 mg, 43%; LCMS (ESI) m/z 1515.80 [M+H]⁺.

### Compound 46

### (2R,4S,5R,6R)-6-((1R,2R)-1,2-dihydroxy-3-(2-(4-(1-(37-oxo-41-((3aS,4S,6aR)-2-oxohexahydro-1H-thieno[3,4-d]imidazol-4-yl)-3,6,9,12,15,18,21,24,27,30,33-undecaoxa-36-azahentetracontyl)-1H-1,2,3-triazol-4-yl)phenyl)acetamido)propyl)-4-hydroxy-5-(2-hydroxyacetamido)-2-((6-(4-((2-methoxyethoxy)methyl)-1H-1,2,3-triazol-1-yl)hexyl)oxy)tetrahydro-2H-pyran-2-carboxylic acid (Cpd. 46)

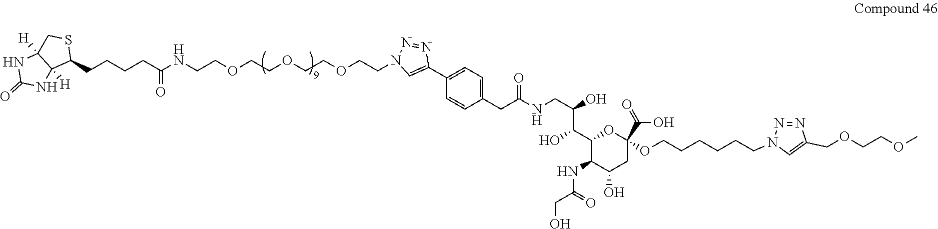

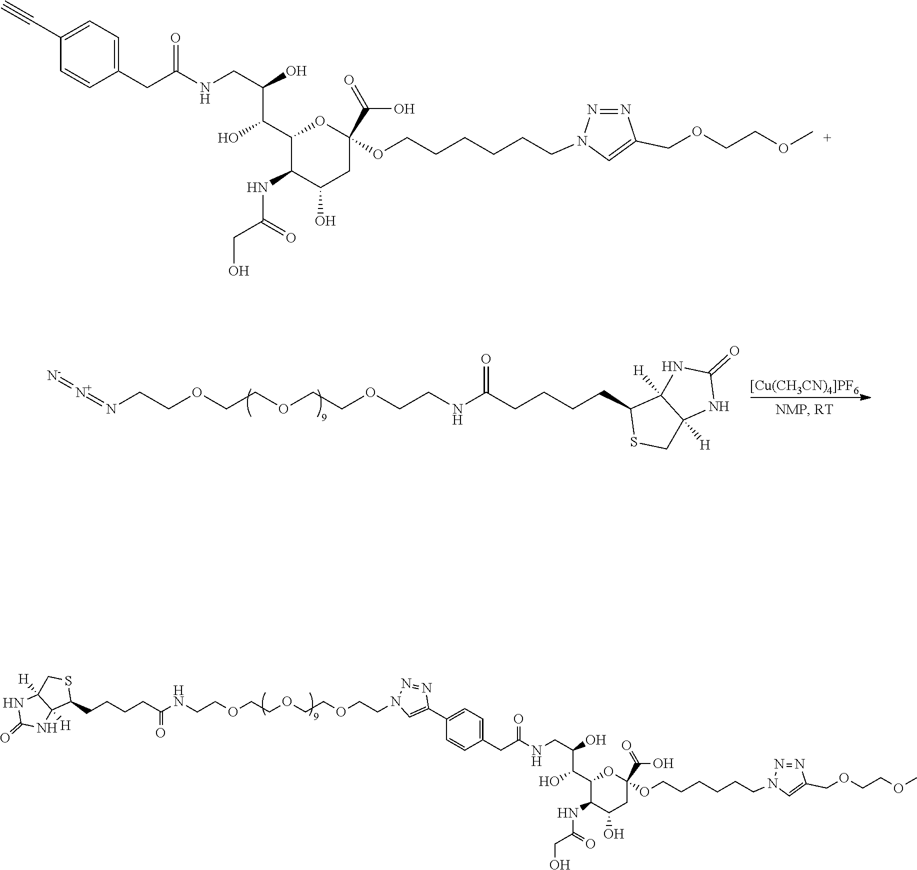

### Synthesis of (2R,4S,5R,6R)-6-((1R,2R)-1,2-dihydroxy-3-(2-(4-(1-(37-oxo-41-((3aS,4S,6aR)-2-oxohexahydro-1H-thieno[3,4-d]imidazol-4-yl)-3,6,9,12,15,18,21,24,27,30,33-undecaoxa-36-azahentetracontyl)-1H-1,2,3-triazol-4-yl)phenyl)acetamido)propyl)-4-hydroxy-5-(2-hydroxyacetamido)-2-((6-(4-((2-methoxyethoxy)methyl)-1H-1,2,3-triazol-1-yl)hexyl)oxy)tetrahydro-2H-pyran-2-carboxylic acid (Cpd. 46)

To a stirred solution of (2R,4S,5R,6R)-6-((1R,2R)-3-(2-(4-ethynylphenyl)acetamido)-1,2-dihydroxypropyl)-4-hydroxy-5-(2-hydroxyacetamido)-2-((6-(4-((2-methoxyethoxy)methyl)-1H-1,2,3-triazol-1-yl)hexyl)oxy)tetrahydro-2H-pyran-2-carboxylic acid (6.87 mg, 9.73 umol) in anhydrous NMP (130 uL) was added Azido-PEG11-Biotin (9.31 mg, 11.7 umol), followed by Tetrakis(acetonitrile)copper(l) hexafluorophosphate (5.44 mg, 14.6 umol). The vial was sealed, and the mixture was stirred for 1 h at room temperature. The mixture was filtered through a 0.45 μm PTFE filter. A few drops of acetic acid were added to the filtrate. The filtrate was purified by preparatory HPLC using acetonitrile and water (+0.05% TFA) as eluents. The fractions containing the desired product were lyophilized to afford Cpd. 46 as a white solid. Yield: 9.85 mg, 67%; LCMS (ESI) m/z 1502.8 [M+H]⁺. ¹H NMR (500 MHz, DMSO-d₆): 8.47 (s, 1H), 8.07 (s, 1H), 7.97 (t, J=5.5 Hz, 1H), 7.83 (br, 1H), 7.82 (t, J=5.5 Hz, 1H), 7.74 (d, J=8.0 Hz, 2H), 7.32 (d, J=8.0 Hz, 2H), 6.40 (s, 1H), 6.34 (s, 1H), 5.52 (t, J=5.5 Hz, 1H), 4.94 (br, 1H), 4.71 (d, J=5.0 Hz, 1H), 4.55 (t, J=5.0 Hz, 2H), 4.50 (s, 2H), 4.31-4.29 (m, 1H), 4.30 (t, J=7.0 Hz, 2H), 4.13-4.11 (m, 1H), 3.91-3.83 (m, 4H), 3.81-3.76 (m, 1H), 3.70-3.66 (m, 1H), 3.64-3.60 (m, 1H), 3.57-3.53 (m, 6H), 3.50-3.46 (m, 42H), 3.45-3.43 (m, 2H), 3.39 (t, J=6.0 Hz, 2H), 3.28-3.27 (m, 1H), 3.22 (s, 3H), 3.21-3.18 (m, 1H), 3.19-3.16 (m, 2H), 3.11-3.07 (m, 1H), 2.96-2.90 (m, 1H), 2.81 (dd, J=12.5 Hz, 5.0 Hz, 1H), 2.57 (d, J=12.5 Hz, 1H), 2.06 (t, J=7.5 Hz, 2H), 1.80-1.74 (m, 2H), 1.64-1.57 (m, 1H), 1.52-1.40 (m, 6H), 1.32-1.23 (m, 4H), 1.22-1.17 (m, 2H).

### Compound 47

### (2R,4S,5R,6R)-6-((1R,2R)-1,2-dihydroxy-3-(2-(4-(1-(37-oxo-41-((3aS,4S,6aR)-2-oxohexahydro-1H-thieno[3,4-d]imidazol-4-yl)-3,6,9,12,15,18,21,24,27,30,33-undecaoxa-36-azahentetracontyl)-1H-1,2,3-triazol-4-yl)phenyl)acetamido)propyl)-4-hydroxy-5-(2-hydroxyacetamido)-2-((6-(1-(2-(2-methoxyethoxy)ethyl)-1H-1,2,3-triazol-4-yl)hexyl)oxy)tetrahydro-2H-pyran-2-carboxylic acid (Cpd. 47)

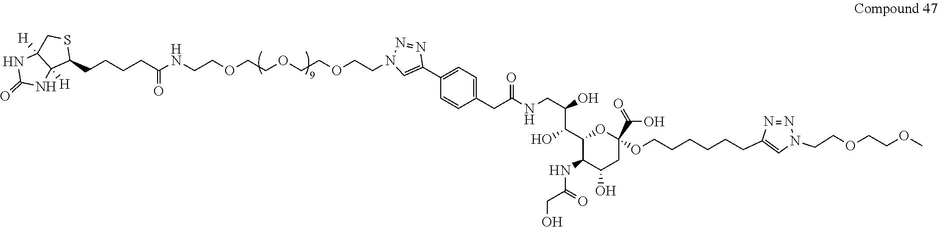

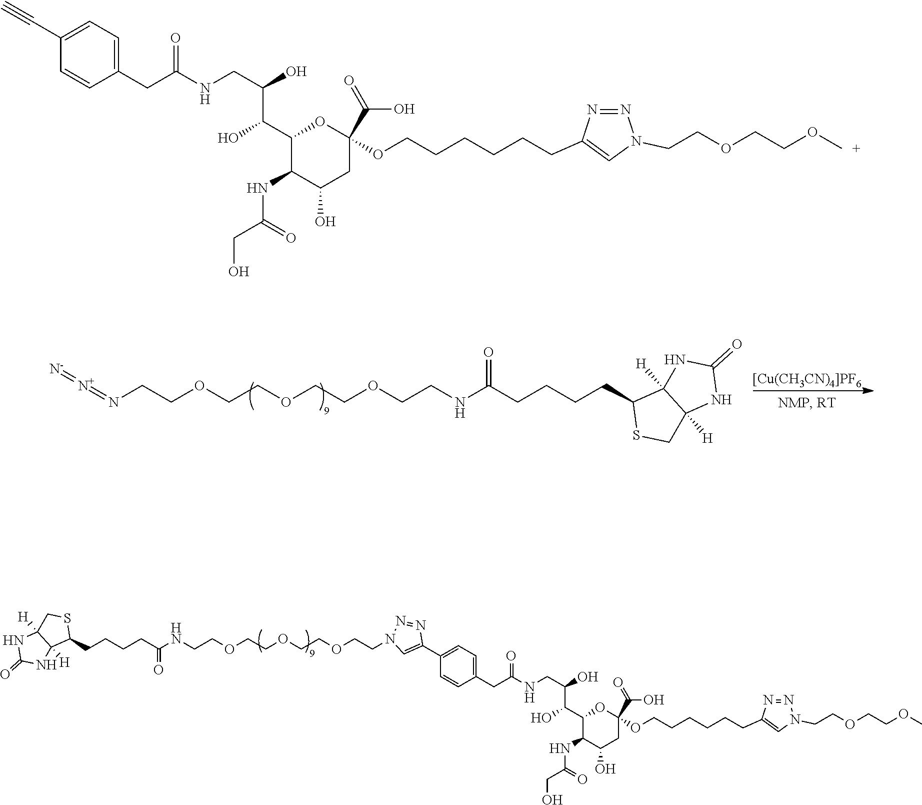

### Synthesis of (2R,4S,5R,6R)-6-((1R,2R)-1,2-dihydroxy-3-(2-(4-(1-(37-oxo-41-((3aS,4S,6aR)-2-oxohexahydro-1H-thieno[3,4-d]imidazol-4-yl)-3,6,9,12,15,18,21,24,27,30,33-undecaoxa-36-azahentetracontyl)-1H-1,2,3-triazol-4-yl)phenyl)acetamido)propyl)-4-hydroxy-5-(2-hydroxyacetamido)-2-((6-(1-(2-(2-methoxyethoxy)ethyl)-1H-1,2,3-triazol-4-yl)hexyl)oxy)tetrahydro-2H-pyran-2-carboxylic acid (Cpd. 47)

To a stirred solution of (2R,4S,5R,6R)-6-((1R,2R)-3-(2-(4-ethynylphenyl)acetamido)-1,2-dihydroxypropyl)-4-hydroxy-5-(2-hydroxyacetamido)-2-((6-(1-(2-(2-methoxyethoxy)ethyl)-1H-1,2,3-triazol-4-yl)hexyl)oxy)tetrahydro-2H-pyran-2-carboxylic acid (8.05 mg, 11.2 umol) in anhydrous NMP (150 uL) was added Azido-PEG11-Biotin (10.7 mg, 13.4 umol), followed by Tetrakis(acetonitrile)copper(l) hexafluorophosphate (6.25 mg, 16.8 umol). The vial was sealed, and the mixture was stirred for 1 h at room temperature. The mixture was filtered through a 0.45 μm PTFE filter. A few drops of acetic acid were added to the filtrate. The filtrate was purified by preparatory HPLC using acetonitrile and water (+0.05% TFA) as eluents. The fractions containing the desired product were lyophilized to afford Cpd. 47 as a white solid. Yield: 11.0 mg, 65%; LCMS (ESI) m/z 1517.80 [M+H]⁺. ¹H NMR (500 MHz, DMSO-d6): 8.47 (s, 1H), 7.96 (t, J=5.5 Hz, 1H), 7.83 (br, 1H), 7.82 (t, J=5.5 Hz, 1H), 7.79 (s, 1H), 7.74 (d, J=8.0 Hz, 2H), 7.32 (d, J=8.0 Hz, 2H), 6.40 (s, 1H), 6.34 (s, 1H), 5.52 (t, J=5.5 Hz, 1H), 4.93 (br, 1H), 4.70 (d, J=5.0 Hz, 1H), 4.55 (t, J=5.0 Hz, 2H), 4.44 (t, J=5.0 Hz, 2H), 4.31-4.29 (m, 1H), 4.13-4.11 (m, 1H), 3.92-3.83 (m, 4H), 3.81-3.78 (m, 1H), 3.77 (t, J=5.0 Hz, 2H), 3.70-3.66 (m, 1H), 3.64-3.60 (m, 1H), 3.56-3.53 (m, 4H), 3.53-3.46 (m, 44H), 3.39-3.37 (m, 4H), 3.29-3.27 (m, 1H), 3.20 (s, 3H), 3.20-3.18 (m, 1H), 3.19-3.16 (m, 2H), 3.11-3.07 (m, 1H), 2.97-2.91 (m, 1H), 2.81 (dd, J=12.5 Hz, 5.0 Hz, 1H), 2.58 (t, J=7.5 Hz, 2H), 2.57 (d, J=12.5 Hz, 1H), 2.06 (t, J=7.5 Hz, 2H), 1.63-1.54 (m, 3H), 1.51-1.42 (m, 6H), 1.31-1.24 (m, 6H).

### Compound 48

### (2R,4S,5R,6R)-6-((1R,2R)-1,2-dihydroxy-3-(2-(4-(1-(37-oxo-41-((3aS,4S,6aR)-2-oxohexahydro-1H-thieno[3,4-d]imidazol-4-yl)-3,6,9,12,15,18,21,24,27,30,33-undecaoxa-36-azahentetracontyl)-1H-1,2,3-triazol-4-yl)phenyl)acetamido)propyl)-4-hydroxy-5-(2-hydroxyacetamido)-2-((6-pentanamidohexyl)oxy)tetrahydro-2H-pyran-2-carboxylic acid (Cpd. 48)

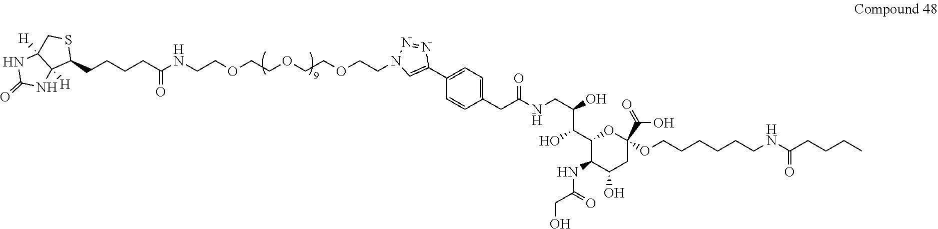

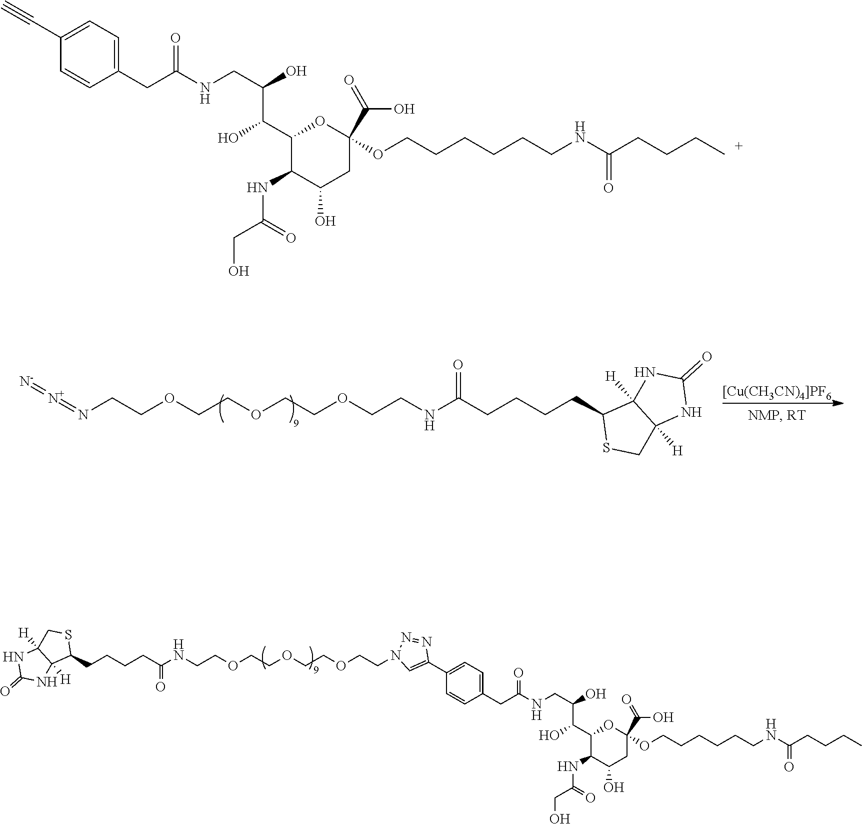

### Synthesis of (2R,4S,5R,6R)-6-((1R,2R)-1,2-dihydroxy-3-(2-(4-(1-(37-oxo-41-((3aS,4S,6aR)-2-oxohexahydro-1H-thieno[3,4-d]imidazol-4-yl)-3,6,9,12,15,18,21,24,27,30,33-undecaoxa-36-azahentetracontyl)-1H-1,2,3-triazol-4-yl)phenyl)acetamido)propyl)-4-hydroxy-5-(2-hydroxyacetamido)-2-((6-pentanamidohexyl)oxy)tetrahydro-2H-pyran-2-carboxylic acid (Cpd. 48)

To a stirred solution of (2R,4S,5R,6R)-6-((1R,2R)-3-(2-(4-ethynylphenyl)acetamido)-1,2-dihydroxypropyl)-4-hydroxy-5-(2-hydroxyacetamido)-2-((6-pentanamidohexyl)oxy)tetrahydro-2H-pyran-2-carboxylic acid (5.23 mg, 8.05 umol) in anhydrous NMP (107 uL) was added Azido-PEG11-Biotin (7.70 mg, 9.66 umol), followed by Tetrakis(acetonitrile)copper(l) hexafluorophosphate (4.50 mg, 12.1 umol). The vial was sealed, and the mixture was stirred for 1 h at room temperature. The mixture was filtered through a 0.45 μm PTFE filter. A few drops of acetic acid were added to the filtrate. The filtrate was purified by preparatory HPLC using acetonitrile and water (+0.05% TFA) as eluents. The fractions containing the desired product were lyophilized to afford Cpd. 48 as a white solid. Yield: 8.51 mg, 73%; LCMS (ESI) m/z 724.10 [M+2H]²⁺. ¹H NMR (500 MHz, DMSO-d₆): 8.47 (s, 1H), 7.97 (t, J=5.5 Hz, 1H), 7.83 (br, 1H), 7.82 (t, J=5.5 Hz, 1H), 7.74 (d, J=8.0 Hz, 2H), 7.71 (br, 1H), 7.32 (d, J=8.0 Hz, 2H), 6.40 (s, 1H), 6.34 (s, 1H), 5.52 (t, J=5.5 Hz, 1H), 4.92 (br, 1H), 4.70 (d, J=5.0 Hz, 1H), 4.56 (t, J=5.0 Hz, 2H), 4.31-4.29 (m, 1H), 4.13-4.11 (m, 1H), 3.92-3.83 (m, 4H), 3.80-3.76 (m, 1H), 3.77 (t, J=5.0 Hz, 2H), 3.70-3.66 (m, 1H), 3.64-3.59 (m, 1H), 3.56-3.53 (m, 4H), 3.53-3.46 (m, 42H), 3.38 (t, J=5.5 Hz, 2H), 3.29-3.27 (m, 1H), 3.20-3.16 (m, 3H), 3.11-3.07 (m, 1H), 3.01-2.97 (m, 2H), 2.97-2.91 (m, 1H), 2.81 (dd, J=12.5 Hz, 5.0 Hz, 1H), 2.57 (d, J=12.5 Hz, 1H), 2.06 (t, J=7.5 Hz, 2H), 2.03 (t, J=7.5 Hz, 2H), 1.64-1.57 (m, 1H), 1.51-1.42 (m, 8H), 1.36-1.27 (m, 4H), 1.26-1.21 (m, 6H), 0.84 (t, J=7.5 Hz, 3H).

### Compound 49

### (2R,4S,5R,6R)-2-(benzyloxy)-6-((1R,2R)-3-(2-(4-chlorophenyl)acetamido)-1,2-dihydroxypropyl)-4-hydroxy-5-(3-(1-(1-(37-oxo-41-((3aS,4S,6aR)-2-oxohexahydro-1H-thieno[3,4-d]imidazol-4-yl)-3,6,9,12,15,18,21,24,27,30,33-undecaoxa-36-azahentetracontyl)-1H-1,2,3-triazol-4-yl)-2,5,8,11,14-pentaoxahexadecan-16-yl)ureido)tetrahydro-2H-pyran-2-carboxylic acid (Cpd. 49)

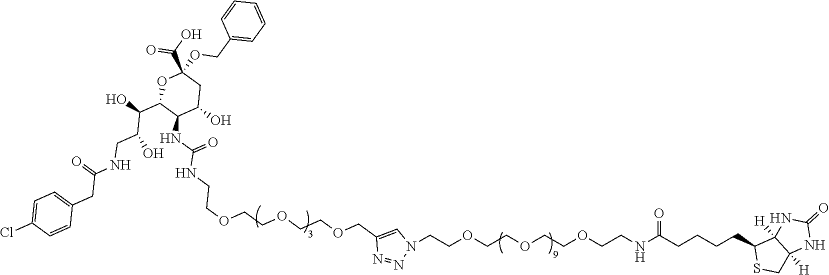

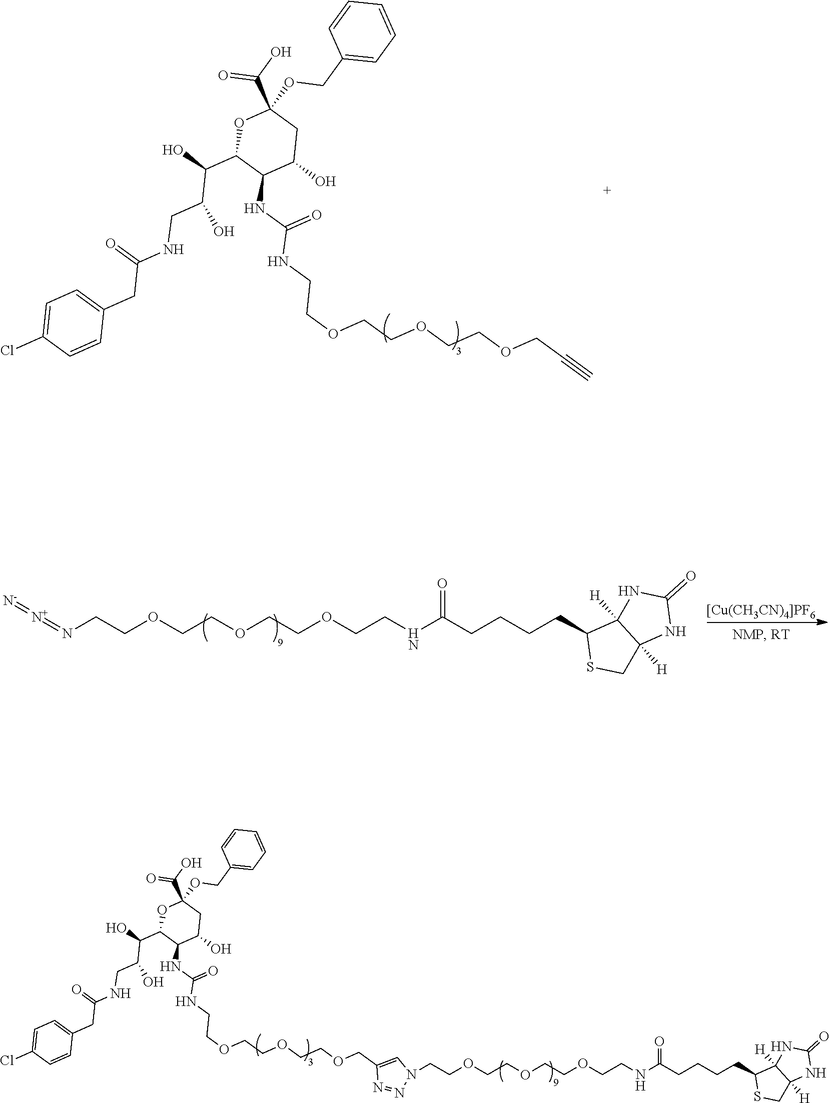

### Synthesis of (2R,4S,5R,6R)-2-(benzyloxy)-6-((1R,2R)-3-(2-(4-chlorophenyl)acetamido)-1,2-dihydroxypropyl)-4-hydroxy-5-(3-(1-(1-(37-oxo-41-((3aS,4S,6aR)-2-oxohexahydro-1H-thieno[3,4-d]imidazol-4-yl)-3,6,9,12,15,18,21,24,27,30,33-undecaoxa-36-azahentetracontyl)-1H-1,2,3-triazol-4-yl)-2,5,8,11,14-pentaoxahexadecan-16-yl)ureido)tetrahydro-2H-pyran-2-carboxylic acid (Cpd. 49)

To a stirred solution of (2R,4S,5R,6R)-5-(3-(3,6,9,12,15-pentaoxaoctadec-17-yn-1-yl)ureido)-2-(benzyloxy)-6-((1R,2R)-3-(2-(4-chlorophenyl)acetamido)-1,2-dihydroxypropyl)-4-hydroxytetrahydro-2H-pyran-2-carboxylic acid (5.02 mg, 6.20 umol) in anhydrous NMP (83 uL) was added Azido-PEG11-Biotin (5.93 mg, 7.43 umol), followed by Tetrakis(acetonitrile)copper(l) hexafluorophosphate (3.46 mg, 9.29 umol). The vial was sealed, and the mixture was stirred for 1 hour at room temperature. The mixture was filtered through a 0.45 μm PTFE filter. A few drops of acetic acid were added to the filtrate. The filtrate was purified by preparatory HPLC using acetonitrile and water (+0.05% TFA) as eluents. The fractions containing the desired product were lyophilized to afford Cpd. 49 as a white solid. Yield: 6.18 mg, 62%; LCMS (ESI) m/z 1606.80 [M+H]⁺. ¹H NMR (500 MHz, DMSO-d₆): 8.04 (s, 1H), 7.95 (t, J=5.5 Hz, 1H), 7.81 (t, J=5.5 Hz, 1H), 7.35-7.25 (m, 9H), 6.40 (s, 1H), 6.34 (s, 1H), 6.21-6.19 (m, 2H), 5.37 (br, 1H), 5.10 (br, 1H), 4.70 (d, J=12.0 Hz, 1H), 4.51 (s, 2H), 4.50 (t, J=5.0 Hz, 2H), 4.43 (d, J=12.0 Hz, 1H), 4.31-4.29 (m, 1H), 4.13-4.11 (m, 1H), 3.81 (t, J=5.5 Hz, 2H), 3.75-3.71 (m, 1H), 3.58-3.54 (m, 3H), 3.53-3.46 (m, 55H), 3.44 (s, 2H), 3.42-3.38 (m, 6H), 3.29-3.27 (m, 1H), 3.20-3.16 (m, 4H), 3.11-3.07 (m, 1H), 2.99-2.95 (m, 1H), 2.83-2.80 (dd, J=12.5, 5.0 Hz, 1H), 2.60-2.56 (m, 2H), 2.06 (t, J=7.5 Hz, 2H), 1.64-1.57 (m, 2H), 1.52-1.42 (m, 3H), 1.33-1.24 (m, 2H).

### Compound 50

### (2R,4S,5R,6R)-2-((5-carboxypentyl)oxy)-6-((1R,2R)-3-(2-(4-chlorophenyl)acetamido)-1,2-dihydroxypropyl)-5-(2-hydroxyacetamido)-4-((1-(37-oxo-41-((3aS,4S,6aR)-2-oxohexahydro-1H-thieno[3,4-d]imidazol-4-yl)-3,6,9,12,15,18,21,24,27,30,33-undecaoxa-36-azahentetracontyl)-1H-1,2,3-triazol-4-yl)methoxy)tetrahydro-2H-pyran-2-carboxylic acid (Cpd. 50)

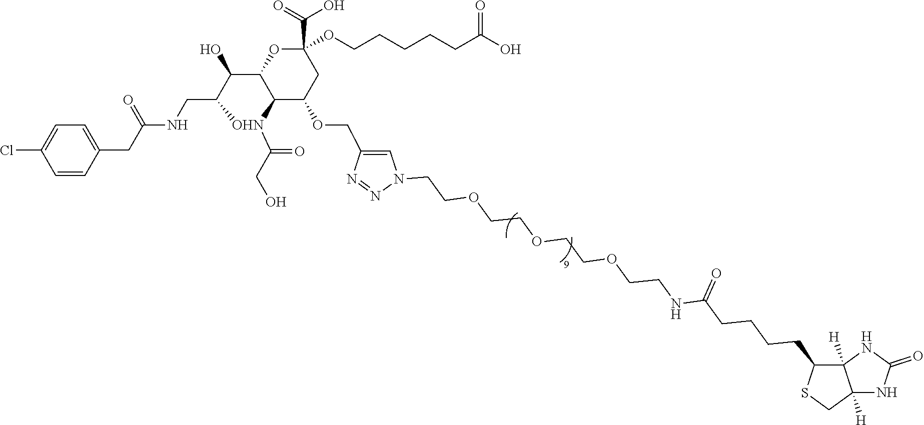

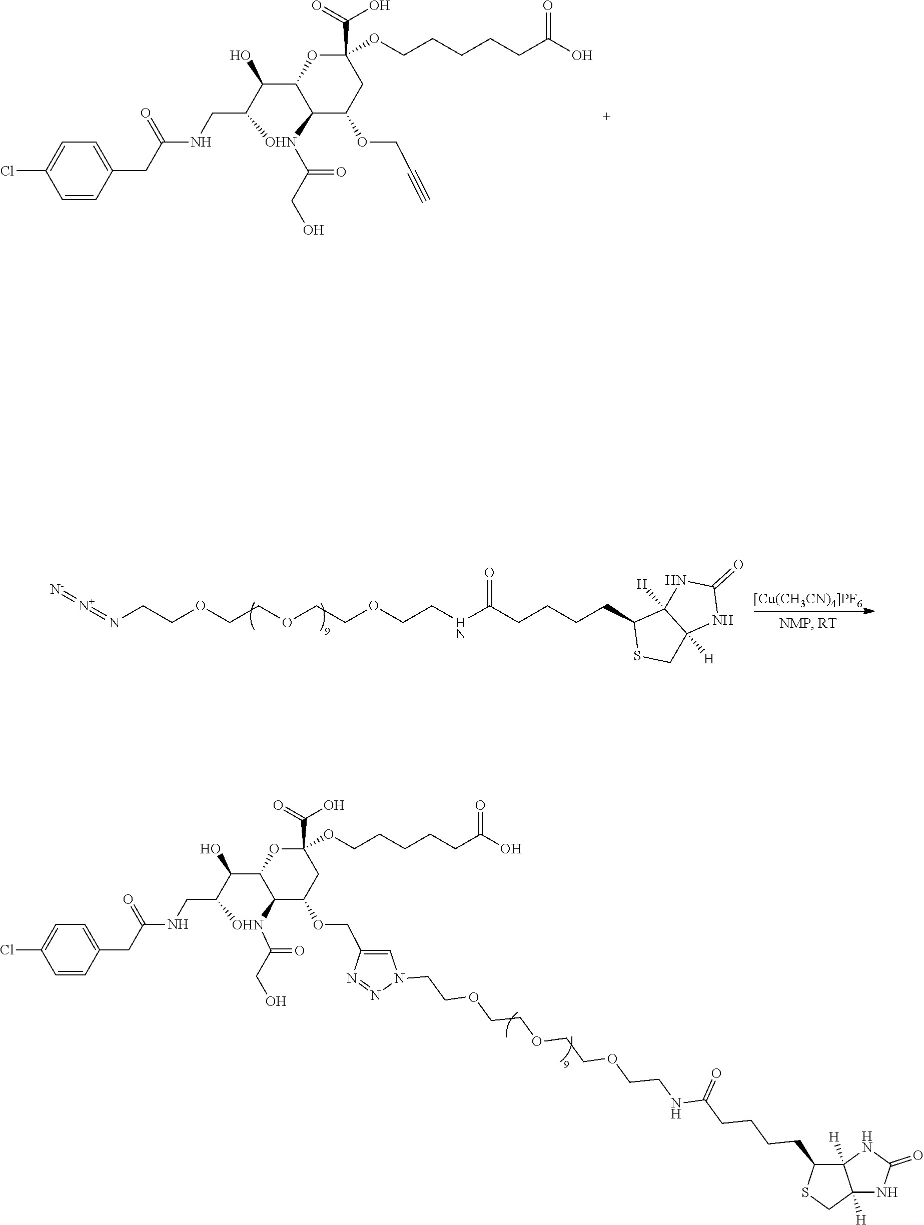

### Synthesis of (2R,4S,5R,6R)-2-((5-carboxypentyl)oxy)-6-((1R,2R)-3-(2-(4-chlorophenyl)acetamido)-1,2-dihydroxypropyl)-5-(2-hydroxyacetamido)-4-((1-(37-oxo-41-((3aS,4S,6aR)-2-oxohexahydro-1H-thieno[3,4-d]imidazol-4-yl)-3,6,9,12,15,18,21,24,27,30,33-undecaoxa-36-azahentetracontyl)-1H-1,2,3-triazol-4-yl)methoxy)tetrahydro-2H-pyran-2-carboxylic acid (Cpd. 50)

To a stirred solution of (2R,4S,5R,6R)-2-((5-carboxypentyl)oxy)-6-((1R,2R)-3-(2-(4-chlorophenyl)acetamido)-1,2-dihydroxypropyl)-5-(2-hydroxyacetamido)-4-(prop-2-yn-1-yloxy)tetrahydro-2H-pyran-2-carboxylic acid (5.60 mg, 8.90 umol) in anhydrous NMP (120 uL) was added Azido-PEG11-Biotin (8.51 mg, 10.7 umol), followed by Tetrakis(acetonitrile)copper(l) hexafluorophosphate (4.98 mg, 13.4 umol). The vial was sealed, and the mixture was stirred for 1 hour at room temperature. The mixture was filtered through a 0.45 μm PTFE filter. A few drops of acetic acid were added to the filtrate. The filtrate was purified by preparatory HPLC using acetonitrile and water (+0.05% TFA) as eluents. The fractions containing the desired product were lyophilized to afford Cpd. 50 as a white solid. Yield: 6.33 mg, 50%; LCMS (ESI) m/z 1425.60 [M+H]⁺. ¹H NMR (500 MHz, DMSO-d₆): 11.94 (br, 1H), 7.99 (d, J=8.0 Hz, 1H), 7.95 (t, J=5.5 Hz, 1H), 7.95 (s, 1H), 7.81 (t, J=5.5 Hz, 1H), 7.33 (d, J=8.5 Hz, 2H), 7.27 (d, J=8.5 Hz, 2H), 6.40 (s, 1H), 6.34 (s, 1H), 5.58 (t, J=5.5 Hz, 1H), 4.60 (d, J=5.5 Hz, 1H), 4.60 (d, J=12.0 Hz, 1H), 4.54 (d, J=12.0 Hz, 1H), 4.49 (t, J=5.5 Hz, 2H), 4.31-4.29 (m, 1H), 4.14-4.11 (m, 1H), 3.94-3.88 (m, 1H), 3.87-3.84 (m, 2H), 3.81 (t, J=5.5 Hz, 2H), 3.72-3.68 (m, 1H), 3.67-3.60 (m, 3H), 3.53-3.48 (m, 40H), 3.44 (s, 2H), 3.39 (t, J=6.0 Hz, 2H), 3.36-3.34 (m, 1H), 3.29-3.27 (m, 1H), 3.22-3.19 (m, 1H), 3.20-3.16 (m, 2H), 3.11-3.07 (m, 1H), 2.98-2.92 (m, 1H), 2.83-2.80 (dd, J=12.5, 5.0 Hz, 1H), 2.71-2.66 (m, 1H), 2.57 (d, J=12.5 Hz, 1H), 2.19 (t, J=7.5 Hz, 2H), 2.06 (t, J=7.5 Hz, 2H), 1.63-1.57 (m, 1H), 1.52-1.43 (m, 8H), 1.32-1.23 (m, 4H).

### Compound 51

### (2R,4S,5R,6R)-2-(benzyloxy)-6-((1R,2R)-3-(2-(4-chlorophenyl)acetamido)-1,2-dihydroxypropyl)-5-(2-hydroxyacetamido)-4-((1-(37-oxo-41-((3aS,4S,6aR)-2-oxohexahydro-1H-thieno[3,4-d]imidazol-4-yl)-3,6,9,12,15,18,21,24,27,30,33-undecaoxa-36-azahentetracontyl)-1H-1,2,3-triazol-4-yl)methoxy)tetrahydro-2H-pyran-2-carboxylic acid (Cpd. 51)

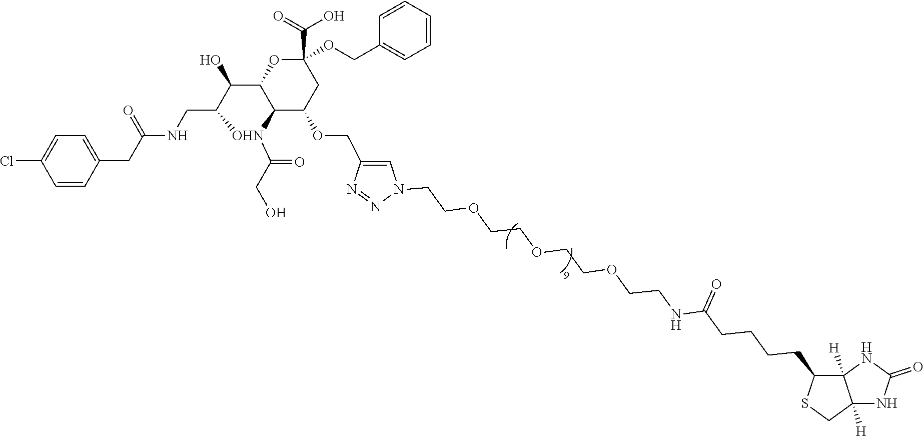

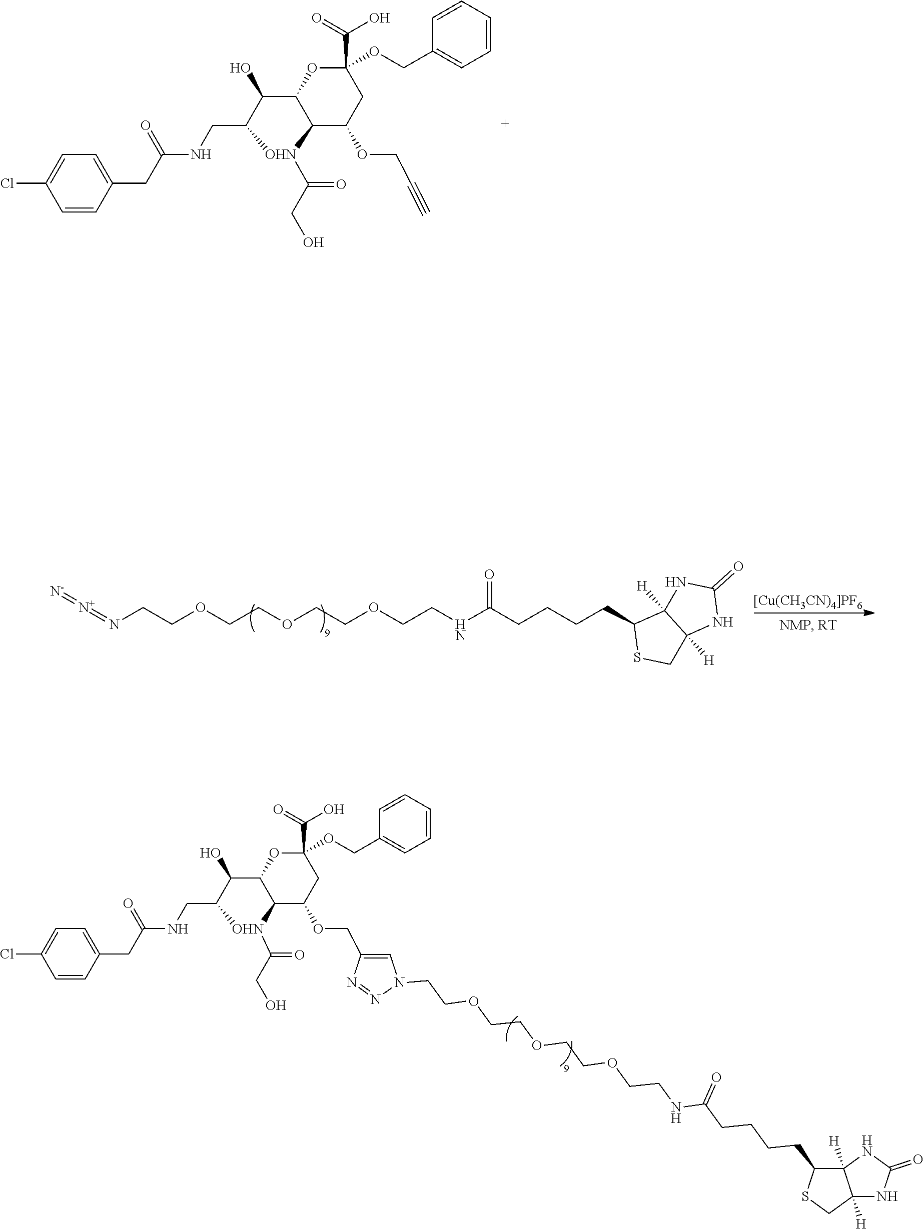

### Synthesis of (2R,4S,5R,6R)-2-(benzyloxy)-6-((1R,2R)-3-(2-(4-chlorophenyl)acetamido)-1,2-dihydroxypropyl)-5-(2-hydroxyacetamido)-4-((1-(37-oxo-41-((3aS,4S,6aR)-2-oxohexahydro-1H-thieno[3,4-d]imidazol-4-yl)-3,6,9,12,15,18,21,24,27,30,33-undecaoxa-36-azahentetracontyl)-1H-1,2,3-triazol-4-yl)methoxy)tetrahydro-2H-pyran-2-carboxylic acid (Cpd. 51)

To a stirred solution of (2R,4S,5R,6R)-2-(benzyloxy)-6-((1R,2R)-3-(2-(4-chlorophenyl)acetamido)-1,2-dihydroxypropyl)-5-(2-hydroxyacetamido)-4-(prop-2-yn-1-yloxy)tetrahydro-2H-pyran-2-carboxylic acid (4.50 mg, 7.44 umol) in anhydrous NMP (100 uL) was added Azido-PEG11-Biotin (7.11 mg, 8.93 umol), followed by Tetrakis(acetonitrile)copper(l) hexafluorophosphate (4.16 mg, 11.2 umol). The vial was sealed, and the mixture was stirred for 1 hour at room temperature. The mixture was filtered through a 0.45 μm PTFE filter. A few drops of acetic acid were added to the filtrate. The filtrate was purified by preparatory HPLC using acetonitrile and water (+0.05% TFA) as eluents. The fractions containing the desired product were lyophilized to afford Cpd. 51 as a white solid. Yield: 5.70 mg, 55%; LCMS (ESI) m/z 1401.60 [M+H]⁺. ¹H NMR (500 MHz, DMSO-d₆): 8.02 (d, J=8.5 Hz, 1H), 7.97 (t, J=5.5 Hz, 1H), 7.95 (s, 1H), 7.81 (t, J=5.5 Hz, 1H), 7.35-7.26 (m, 9H), 6.40 (s, 1H), 6.34 (s, 1H), 5.60 (br, 1H), 4.73 (d, J=12.0 Hz, 1H), 4.64 (br, 1H), 4.62 (d, J=12.0 Hz, 1H), 4.57 (d, J=12.0 Hz, 1H), 4.49 (t, J=5.5 Hz, 2H), 4.46 (d, J=12.0 Hz, 1H), 4.36 (br. 1H), 4.31-4.28 (m, 1H), 4.14-4.11 (m, 1H), 4.01-3.96 (m, 1H), 3.91-3.84 (m, 2H), 3.80 (t, J=5.5 Hz, 2H), 3.77-3.70 (m, 2H), 3.55-3.53 (m, 1H), 3.52-3.47 (m, 41H), 3.44 (s, 2H), 3.39 (t, J=6.0 Hz, 2H), 3.27-3.25 (m, 1H), 3.20-3.16 (m, 2H), 3.11-3.07 (m, 1H), 3.02-2.96 (m, 1H), 2.83-2.80 (dd, J=12.5, 5.0 Hz, 1H), 2.73-2.70 (dd, J=12.5, 5.0 Hz, 1H), 2.57 (d, J=12.5 Hz, 1H), 2.06 (t, J=7.5 Hz, 1H), 1.62-1.58 (m, 2H), 1.52-1.41 (m, 3H), 1.33-1.23 (m, 2H).

### Compound 52

### (2R,4S,5R,6R)-6-((1R,2R)-1,2-dihydroxy-3-(2-(4-(1-(15-oxo-3,6,9,12-tetraoxa-16-azanonadecyl)-1H-1,2,3-triazol-4-yl)phenyl)acetamido)propyl)-4-hydroxy-5-(2-hydroxyacetamido)-2-((6-methoxyhexyl)oxy)tetrahydro-2H-pyran-2-carboxylic acid (Cpd. 52)

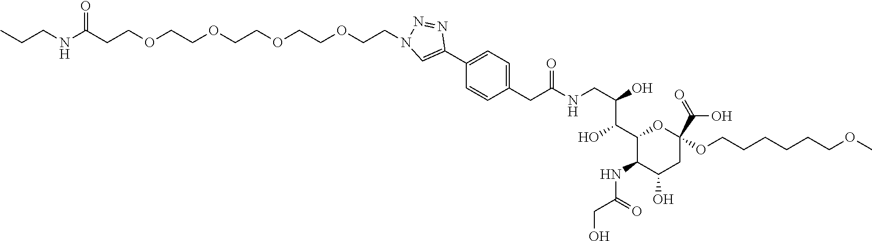 [SMILES: CCCNC(=O)CCOCCOCCOCCOCCn1cc(-c2ccc(CC(=O)NC[C@@H](O)[C@@H](O)[C@@H]3O[C@@](OCCCCCCOC)(C(=O)O)C[C@H](O)[C@H]3NC(=O)CO)cc2)nn1]

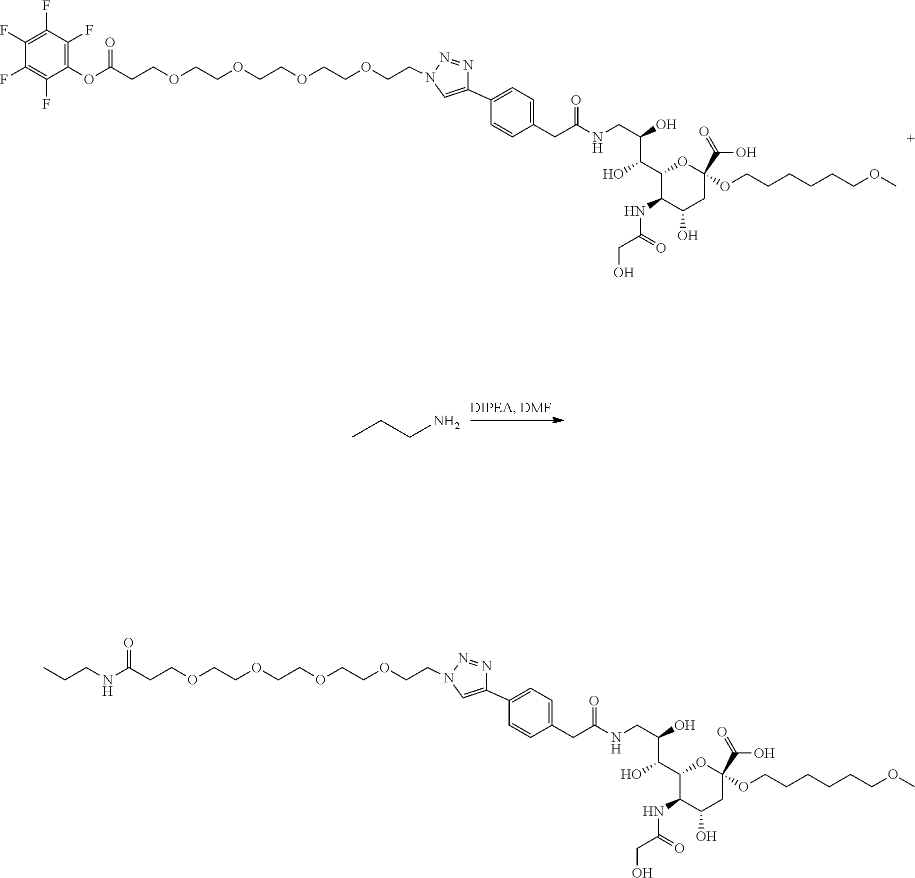

### Synthesis of (2R,4S,5R,6R)-6-((1R,2R)-1,2-dihydroxy-3-(2-(4-(1-(15-oxo-3,6,9,12-tetraoxa-16-azanonadecyl)-1H-1, 2, 3-triazol-4-yl)phenyl)acetamido)propyl)-4-hydroxy-5-(2-hydroxyacetamido)-2-((6-methoxyhexyl)oxy)tetrahydro-2H-pyran-2-carboxylic acid (Cpd. 52)

To a stirred solution of (2R,4S,5R,6R)-6-((1R,2R)-1,2-dihydroxy-3-(2-(4-(1-(15-oxo-15-(perfluorophenoxy)-3,6,9,12-tetraoxapentadecyl)-1H-1,2,3-triazol-4-yl)phenyl)acetamido)propyl)-4-hydroxy-5-(2-hydroxyacetamido)-2-((6-methoxyhexyl)oxy)tetrahydro-2H-pyran-2-carboxylic acid (5.92 mg, 5.70 umol) in anhydrous N,N-dimethylformamide (143 uL), a solution of propylamine (1.41 uL, 17.1 umol)) and N,N-diisopropylethylamine (3.97 uL, 22.8 umol) in N,N-dimethylformamide (143 uL) was added. The vial was sealed, and the mixture was stirred for 4 hours at room temperature. The mixture was filtered through a 0.45 μm PTFE filter. The filtrate was purified by preparatory HPLC using acetonitrile and water (+0.05% TFA) as eluents. The fractions containing the desired product were lyophilized to afford Cpd. 52 as a white solid. Yield: 4.40 mg, 84%; LCMS m/z 913.50 [M+H]⁺. ¹H NMR (500 MHz, DMSO-d₆): 8.47 (s, 1H), 7.95 (t, J=5.5 Hz, 1H), 7.81 (d, J=7.5 Hz, 1H), 7.77 (t, J=5.5 Hz, 1H), 7.74 (d, J=8.0 Hz, 2H), 7.32 (d, J=8.0 Hz, 2H), 5.52 (t, J=5.5 Hz, 1H), 4.91 (d, J=5.5 Hz, 1H), 4.70 (d, J=5.0 Hz, 1H), 4.55 (t, J=5.0 Hz, 2H), 3.91-3.83 (m, 4H), 3.80-3.75 (m, 1H), 3.69-3.65 (m, 1H), 3.64-3.59 (m, 1H), 3.57-3.52 (m, 7H), 3.50-3.43 (m, 14H), 3.27 (t, J=6.5 Hz, 2H), 3.20-3.18 (m, 1H), 3.19 (s, 3H), 2.98 (dd, J=7.0 Hz, 2H), 2.95-2.90 (m, 1H), 2.54-2.51 (m, 1H), 2.27 (t, J=6.5 Hz, 2H), 1.46-1.42 (m, 4H), 1.38 (qt, J=7.5 Hz, 2H), 1.27-1.23 (m, 4H), 0.82 (t, J=7.5 Hz, 3H).

### Compound 53

### (2R,4S,5R,6R)-6-((1R,2R)-1,2-dihydroxy-3-(2-(4-(1-(15-oxo-3,6,9,12-tetraoxa-16-azanonadecyl)-1H-1,2,3-triazol-4-yl)phenyl)acetamido)propyl)-4-hydroxy-5-(2-hydroxyacetamido)-2-((6-((2-((2-methoxyethyl)amino)-3,4-dioxocyclobut-1-en-1-yl)amino)hexyl)oxy)tetrahydro-2H-pyran-2-carboxylic acid (Cpd. 53)

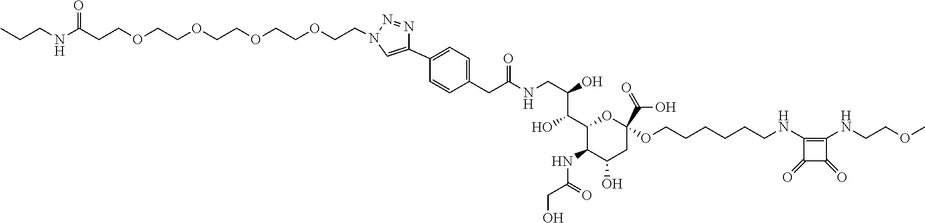 [SMILES: CCCNC(=O)CCOCCOCCOCCOCCn1cc(-c2ccc(CC(=O)NC[C@@H](O)[C@@H](O)[C@@H]3O[C@@](OCCCCCCNc4c(NCCOC)c(=O)c4=O)(C(=O)O)C[C@H](O)[C@H]3NC(=O)CO)cc2)nn1]

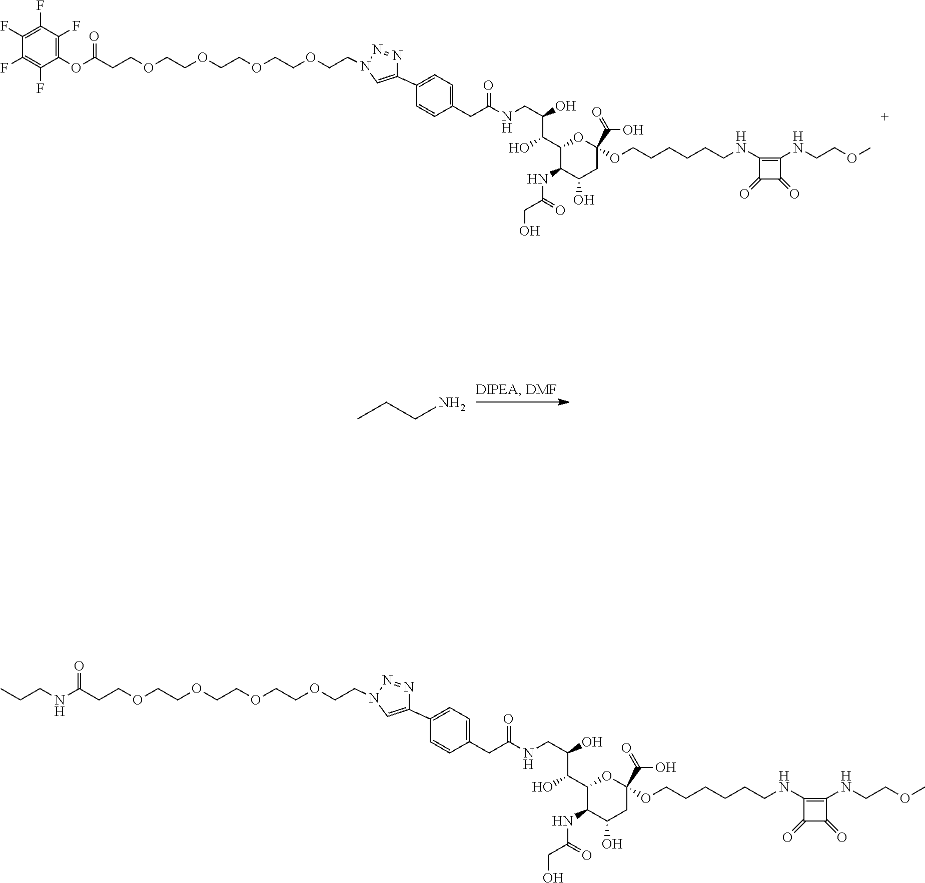

### Synthesis of (2R,4S,5R,6R)-6-((1R,2R)-1,2-dihydroxy-3-(2-(4-(1-(15-oxo-3,6,9,12-tetraoxa-16-azanonadecyl)-1H-1,2,3-triazol-4-yl)phenyl)acetamido)propyl)-4-hydroxy-5-(2-hydroxyacetamido)-2-((6-((2-((2-methoxyethyl)amino)-3,4-dioxocyclobut-1-en-1-yl)amino)hexyl)oxy)tetrahydro-2H-pyran-2-carboxylic acid (Cpd. 53)

To a stirred solution of (2R,4S,5R,6R)-6-((1R,2R)-1,2-dihydroxy-3-(2-(4-(1-(15-oxo-15-(perfluorophenoxy)-3,6,9,12-tetraoxapentadecyl)-1H-1,2,3-triazol-4-yl)phenyl)acetamido)propyl)-4-hydroxy-5-(2-hydroxyacetamido)-2-((6-((2-((2-methoxyethyl)amino)-3,4-dioxocyclobut-1-en-1-yl)amino)hexyl)oxy)tetrahydro-2H-pyran-2-carboxylic acid (2.56 mg, 2.18 umol) in anhydrous N,N-dimethylformamide (54 uL), a solution of propylamine (0.72 uL, 8.71 umol)) and N,N-diisopropylethylamine (1.52 uL, 8.71 umol) in N,N-dimethylformamide (54 uL) was added. The vial was sealed, and the mixture was stirred for 4 hours at room temperature. The mixture was filtered through a 0.45 μm PTFE filter. The filtrate was purified by preparatory HPLC using acetonitrile and water (+0.05% TFA) as eluents. The fractions containing the desired product were lyophilized to afford Cpd. 53 as a white solid. Yield: 1.86 mg, 81%; LCMS m/z 1051.50 [M+H]⁺. ¹H NMR (500 MHz, DMSO-d₆): 8.46 (s, 1H), 7.96 (br, 1H), 7.80 (br, 1H), 7.77 (t, J=5.5 Hz, 1H), 7.74 (d, J=8.0 Hz, 2H), 7.32 (d, J=8.0 Hz, 2H), 5.51 (t, J=5.5 Hz, 1H), 4.88 (br, 1H), 4.70 (d, J=4.5 Hz, 1H), 4.55 (t, J=5.5 Hz, 2H), 3.91-3.83 (m, 4H), 3.78-3.72 (m, 1H), 3.70-3.64 (m, 3H), 3.62-3.58 (m, 1H), 3.57-3.53 (m, 6H), 3.50-3.43 (m, 16H), 3.27 (s, 3H), 3.19-3.15 (m, 1H), 2.98 (t, J=7.0 Hz, 2H), 2.97 (t, J=7.0 Hz, 2H), 2.93-2.88 (m, 1H), 2.52-2.50 (m, 1H), 2.27 (t, J=6.5 Hz, 2H), 1.54-1.35 (m, 5H), 1.37 (qt, J=7.5 Hz, 2H), 1.32-1.19 (m, 4H), 0.82 (t, J=7.5 Hz, 3H).

### Compound 54

### (2R,4S,5R,6R)-6-((1R,2R)-1,2-dihydroxy-3-(2-(4-(1-(15-oxo-3,6,9,12-tetraoxa-16-azanonadecyl)-1H-1,2,3-triazol-4-yl)phenyl)acetamido)propyl)-4-hydroxy-5-(2-hydroxyacetamido)-2-((6-(1-(2-(2-methoxyethoxy)ethyl)-1H-1,2,3-triazol-4-yl)hexyl)oxy)tetrahydro-2H-pyran-2-carboxylic acid (Cpd. 54)

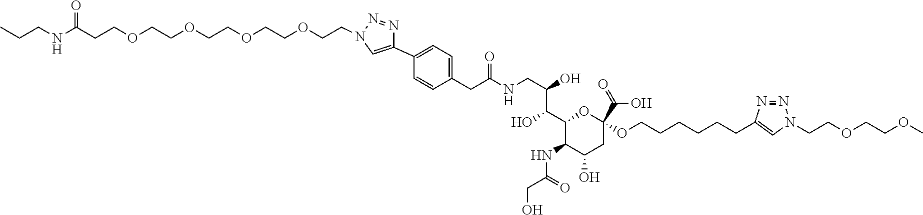 [SMILES: CCCNC(=O)CCOCCOCCOCCOCCn1cc(-c2ccc(CC(=O)NC[C@@H](O)[C@@H](O)[C@@H]3O[C@@](OCCCCCCc4cn(CCOCCOC)nn4)(C(=O)O)C[C@H](O)[C@H]3NC(=O)CO)cc2)nn1]

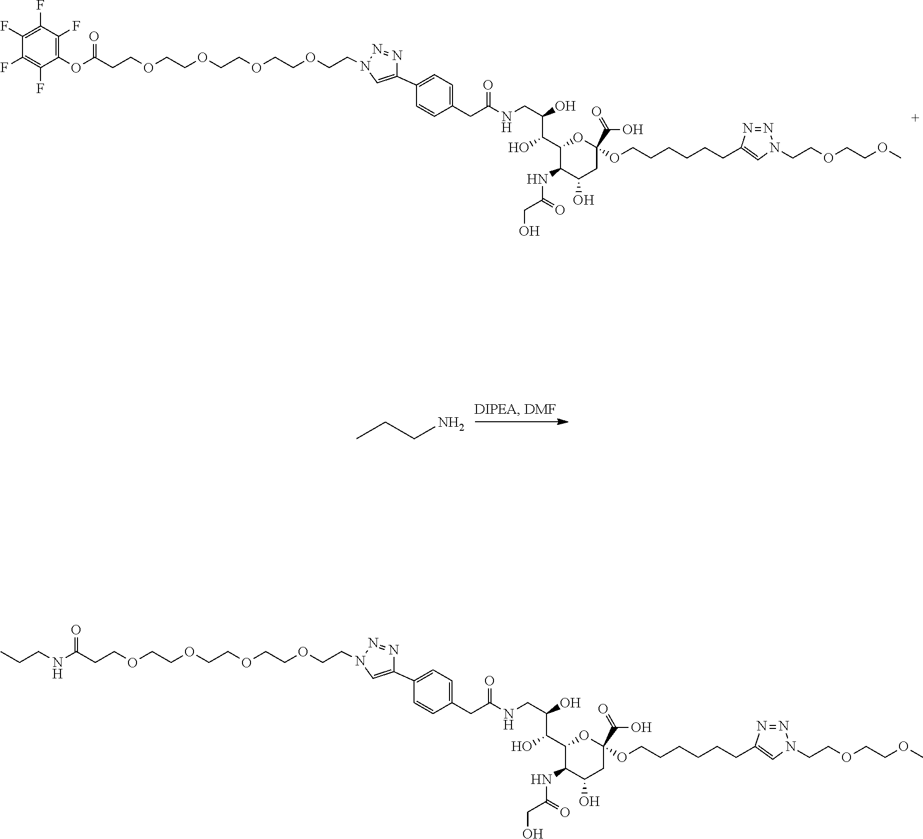

### Synthesis of (2R,4S,5R,6R)-6-((1R,2R)-1,2-dihydroxy-3-(2-(4-(1-(15-oxo-3,6,9,12-tetraoxa-16-azanonadecyl)-1H-1,2,3-triazol-4-yl)phenyl)acetamido)propyl)-4-hydroxy-5-(2-hydroxyacetamido)-2-((6-(1-(2-(2-methoxyethoxy)ethyl)-1H-1,2,3-triazol-4-yl)hexyl)oxy)tetrahydro-2H-pyran-2-carboxylic acid (Cpd. 54)

To a stirred solution of (2R,4S,5R,6R)-6-((1R,2R)-1,2-dihydroxy-3-(2-(4-(1-(15-oxo-15-(perfluorophenoxy)-3,6,9,12-tetraoxapentadecyl)-1H-1,2,3-triazol-4-yl)phenyl)acetamido)propyl)-4-hydroxy-5-(2-hydroxyacetamido)-2-((6-(1-(2-(2-methoxyethoxy)ethyl)-1H-1,2,3-triazol-4-yl)hexyl)oxy)tetrahydro-2H-pyran-2-carboxylic acid (4.95 mg, 4.21 umol) in anhydrous N,N-dimethylformamide (105 uL), a solution of propylamine (1.73 uL, 21.0 umol)) and N,N-diisopropylethylamine (2.93 uL, 16.8 umol) in N,N-dimethylformamide (105 uL) was added. The vial was sealed, and the mixture was stirred for 4 hours at room temperature. The mixture was filtered through a 0.45 μm PTFE filter. The filtrate was purified by preparatory HPLC using acetonitrile and water (+0.05% TFA) as eluents. The fractions containing the desired product were lyophilized to afford Cpd. 54 as a white solid. Yield: 3.29 mg, 74%; LCMS m/z 1052.60 [M+H]⁺. ¹H NMR (500 MHz, DMSO-d₆): 8.46 (s, 1H), 7.96 (t, J=5.5 Hz, 1H), 7.81 (br, 1H), 7.79 (s, 1H), 7.77 (br, 1H), 7.74 (d, J=8.0 Hz, 2H), 7.32 (d, J=8.0 Hz, 2H), 5.51 (t, J=5.5 Hz, 1H), 4.91 (br, 1H), 4.70 (d, J=4.5 Hz, 1H), 4.55 (t, J=5.5 Hz, 2H), 4.44 (t, J=5.5 Hz, 2H), 3.91-3.83 (m, 4H), 3.82-3.77 (m, 1H), 3.77 (t, J=5.5 Hz, 2H), 3.70-3.66 (m, 1H), 3.64-3.60 (m, 1H), 3.57-3.53 (m, 6H), 3.51-3.43 (m, 16H), 3.40-3.38 (m, 2H), 3.22-3.20 (m, 1H), 3.20 (s, 3H), 2.98 (t, J=6.5 Hz, 2H), 2.97 (t, J=6.5 Hz, 2H), 2.97-2.92 (m, 1H), 2.57 (t, J=7.5 Hz, 2H), 2.52-2.50 (m, 1H), 2.27 (t, J=6.5 Hz, 2H), 1.57-1.51 (m, 3H), 1.47-1.41 (m, 2H), 1.37 (qt, J=7.5 Hz, 2H), 1.30-1.24 (m, 4H), 0.82 (t, J=7.5 Hz, 3H).

### Compound 55

### (2R,4S,5R,6R)-6-((1R,2R)-1,2-dihydroxy-3-(2-(4-(1-(15-oxo-3,6,9,12-tetraoxa-16-azanonadecyl)-1H-1,2,3-triazol-4-yl)phenyl)acetamido)propyl)-4-hydroxy-5-(2-hydroxyacetamido)-2-((6-(4-((2-methoxyethoxy)methyl)-1H-1,2,3-triazol-1-yl)hexyl)oxy)tetrahydro-2H-pyran-2-carboxylic acid (Cpd. 55)

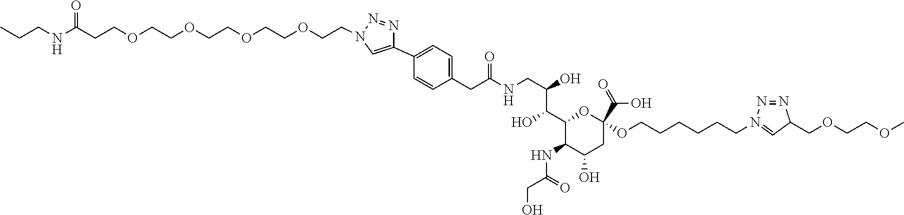

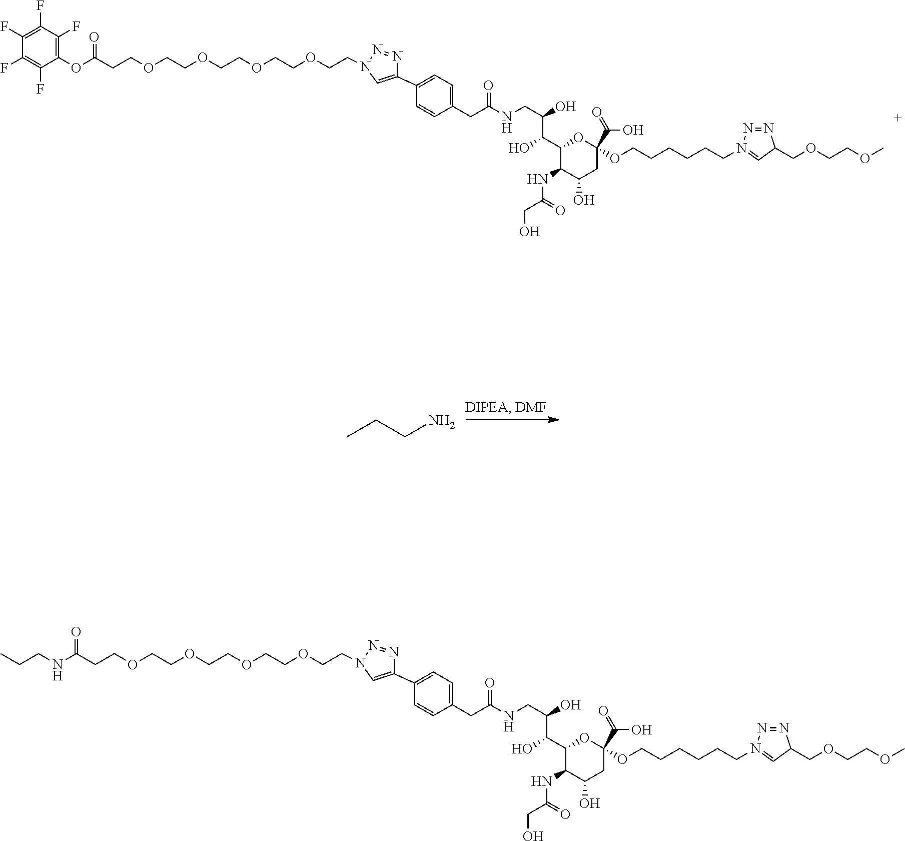

### Synthesis of (2R,4S,5R,6R)-6-((1R,2R)-1,2-dihydroxy-3-(2-(4-(1-(15-oxo-3,6,9,12-tetraoxa-16-azanonadecyl)-1H-1,2,3-triazol-4-yl)phenyl)acetamido)propyl)-4-hydroxy-5-(2-hydroxyacetamido)-2-((6-(4-((2-methoxyethoxy)methyl)-1H-1,2,3-triazol-1-yl)hexyl)oxy)tetrahydro-2H-pyran-2-carboxylic acid (Cpd. 55)

To a stirred solution of (2R,4S,5R,6R)-6-((1R,2R)-1,2-dihydroxy-3-(2-(4-(1-(15-oxo-15-(perfluorophenoxy)-3,6,9,12-tetraoxapentadecyl)-1H-1,2,3-triazol-4-yl)phenyl)acetamido)propyl)-4-hydroxy-5-(2-hydroxyacetamido)-2-((6-(4-((2-methoxyethoxy)methyl)-1H-1,2,3-triazol-1-yl)hexyl)oxy)tetrahydro-2H-pyran-2-carboxylic acid (4.50 mg, 3.87 umol) in anhydrous N,N-dimethylformamide (97 uL), a solution of propylamine (1.59 uL, 19.3 umol)) and N,N-diisopropylethylamine (2.70 uL, 15.5 umol) in N,N-dimethylformamide (97 uL) was added. The vial was sealed, and the mixture was stirred for 4 hours at room temperature. The mixture was filtered through a 0.45 μm PTFE filter. The filtrate was purified by preparatory HPLC using acetonitrile and water (+0.05% TFA) as eluents. The fractions containing the desired product were lyophilized to afford Cpd. 55 as a white solid. Yield: 3.05 mg, 76%; LCMS m/z 1038.50 [M+H]⁺. ¹H NMR (500 MHz, DMSO-d₆): 8.46 (s, 1H), 8.07 (s, 1H), 7.97 (t, J=5.5 Hz, 1H), 7.81 (d, J=7.5 Hz, 1H), 7.77 (t, J=5.5 Hz, 1H), 7.73 (d, J=8.0 Hz, 2H), 7.32 (d, J=8.0 Hz, 2H), 5.51 (t, J=5.5 Hz, 1H), 4.91 (br, 1H), 4.70 (d, J=4.5 Hz, 1H), 4.55 (t, J=5.0 Hz, 2H), 4.50 (s, 2H), 4.30 (t, J=7.0 Hz, 2H), 3.91-3.83 (m, 4H), 3.80-3.75 (m, 1H), 3.70-3.66 (m, 1H), 3.64-3.60 (m, 1H), 3.57-3.53 (m, 7H), 3.50-3.43 (m, 14H), 3.29-3.27 (m, 1H), 3.22 (s, 3H), 3.22-3.19 (m, 1H), 2.98 (t, J=7.0 Hz, 2H), 2.97 (t, J=7.0 Hz, 2H), 2.95-2.90 (m, 1H), 2.54-2.51 (m, 1H), 2.27 (t, J=6.5 Hz, 2H), 1.80-1.74 (m, 2H), 1.51-1.39 (m, 3H), 1.37 (qt, J=7.5 Hz, 2H), 1.30-1.23 (m, 2H), 1.22-1.16 (m, 2H), 0.82 (t, J=7.5 Hz, 3H).

### Compound 56

### (2R,4S,5R,6R)-6-((1R,2R)-1,2-dihydroxy-3-(2-(4-(1-(15-oxo-3,6,9,12-tetraoxa-16-azanonadecyl)-1H-1,2,3-triazol-4-yl)phenyl)acetamido)propyl)-4-hydroxy-5-(2-hydroxyacetamido)-2-((6-pentanamidohexyl)oxy)tetrahydro-2H-pyran-2-carboxylic acid (Cpd. 56)

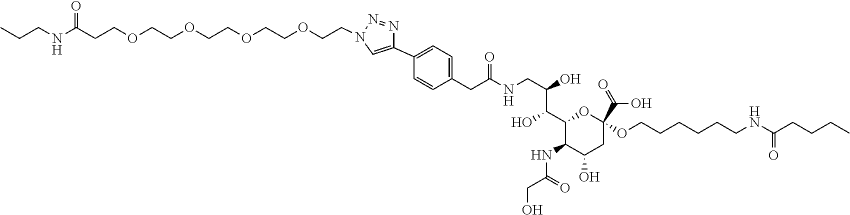 [SMILES: CCCCC(=O)NCCCCCCO[C@]1(C(=O)O)C[C@H](O)[C@@H](NC(=O)CO)[C@H]([C@H](O)[C@H](O)CNC(=O)Cc2ccc(-c3cn(CCOCCOCCOCCOCCC(=O)NCCC)nn3)cc2)O1]

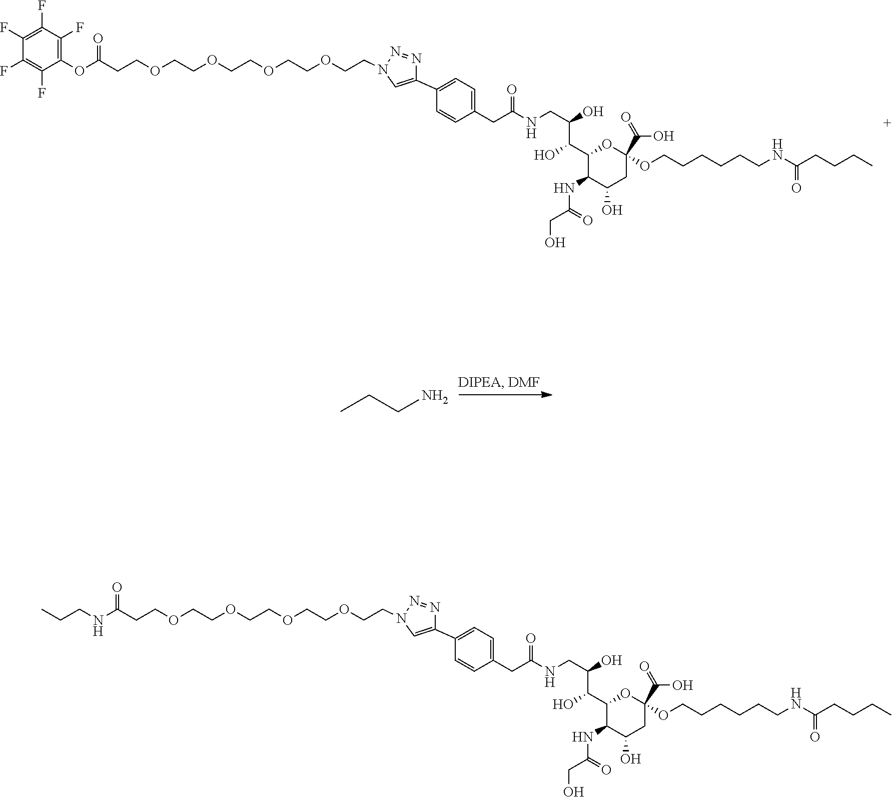

### Synthesis of (2R,4S,5R,6R)-6-((1R,2R)-1,2-dihydroxy-3-(2-(4-(1-(15-oxo-3,6,9,12-tetraoxa-16-azanonadecyl)-1H-1,2,3-triazol-4-yl)phenyl)acetamido)propyl)-4-hydroxy-5-(2-hydroxyacetamido)-2-((6-pentanamidohexyl)oxy)tetrahydro-2H-pyran-2-carboxylic acid (Cpd. 56)

To a stirred solution of (2R,4S,5R,6R)-6-((1R,2R)-1,2-dihydroxy-3-(2-(4-(1-(15-oxo-15-(perfluorophenoxy)-3,6,9,12-tetraoxapentadecyl)-1H-1,2,3-triazol-4-yl)phenyl)acetamido)propyl)-4-hydroxy-5-(2-hydroxyacetamido)-2-((6-pentanamidohexyl)oxy)tetrahydro-2H-pyran-2-carboxylic acid (5.12 mg, 4.62 umol) in anhydrous N,N-dimethylformamide (116 uL), a solution of propylamine (1.90 uL, 23.1 umol)) and N,N-diisopropylethylamine (3.22 uL, 18.5 umol) in N,N-dimethylformamide (116 uL) was added. The vial was sealed, and the mixture was stirred for 4 hours at room temperature. The mixture was filtered through a 0.45 μm PTFE filter. The filtrate was purified by preparatory HPLC using acetonitrile and water (+0.05% TFA) as eluents. The fractions containing the desired product were lyophilized to afford Cpd. 56 as a white solid. Yield: 3.83 mg, 84%; LCMS m/z 982.50 [M+H]⁺. ¹H NMR (500 MHz, DMSO-d₆): 8.47 (s, 1H), 7.96 (t, J=5.5 Hz, 1H), 7.81 (d, J=7.5 Hz, 1H), 7.77 (t, J=5.5 Hz, 1H), 7.74 (d, J=8.0 Hz, 2H), 7.71 (t, J=5.5 Hz, 1H), 7.32 (d, J=8.5 Hz, 2H), 5.51 (t, J=5.5 Hz, 1H), 4.90 (d, J=5.5 Hz, 1H), 4.70 (d, J=4.5 Hz, 1H), 4.56 (t, J=5.5 Hz, 2H), 3.92-3.83 (m, 4H), 3.80-3.75 (m, 1H), 3.70-3.66 (m, 1H), 3.64-3.60 (m, 1H), 3.57-3.53 (m, 5H), 3.50-3.43 (m, 12H), 3.29-3.27 (m, 1H), 3.20-3.17 (m, 1H), 3.00-2.97 (m, 2H), 2.99 (t, J=7.0 Hz, 2H), 2.97 (t, J=7.0 Hz, 2H), 2.95-2.90 (m, 1H), 2.54-2.51 (m, 1H), 2.27 (t, J=6.5 Hz, 2H), 2.03 (t, J=7.5 Hz, 2H), 1.48-1.42 (m, 5H), 1.37-1.33 (m, 2H), 1.37 (qt, J=7.0 Hz, 2H), 1.27-1.20 (m, 6H), 0.84 (t, J=7.5 Hz, 3H), 0.82 (t, J=7.5 Hz, 3H).

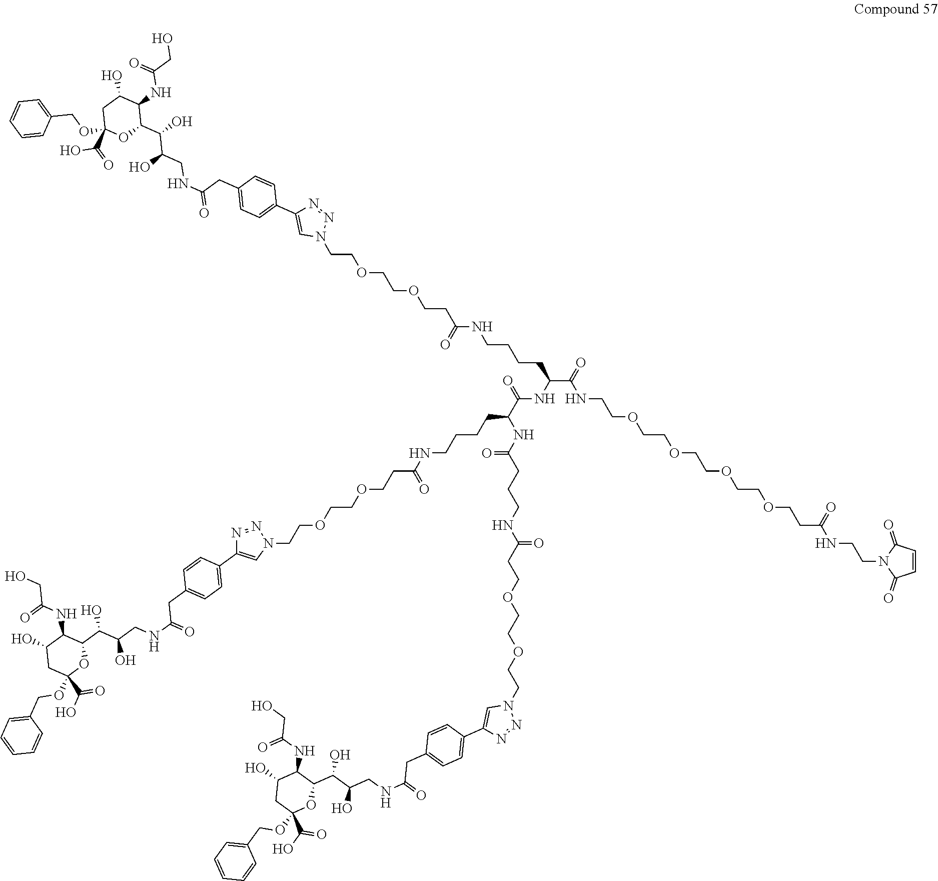 [SMILES: O=C(CCOCCOCCOCCOCCNC(=O)[C@H](CCCCNC(=O)CCOCCOCCn1cc(-c2ccc(CC(=O)NC[C@@H](O)[C@@H](O)[C@@H]3O[C@@](OCc4ccccc4)(C(=O)O)C[C@H](O)[C@H]3NC(=O)CO)cc2)nn1)NC(=O)[C@H](CCCCNC(=O)CCOCCOCCn1cc(-c2ccc(CC(=O)NC[C@@H](O)[C@@H](O)[C@@H]3O[C@@](OCc4ccccc4)(C(=O)O)C[C@H](O)[C@H]3NC(=O)CO)cc2)nn1)NC(=O)CCCNC(=O)CCOCCOCCn1cc(-c2ccc(CC(=O)NC[C@@H](O)[C@@H](O)[C@@H]3O[C@@](OCc4ccccc4)(C(=O)O)C[C@H](O)[C@H]3NC(=O)CO)cc2)nn1)NCCN1C(=O)C=CC1=O]

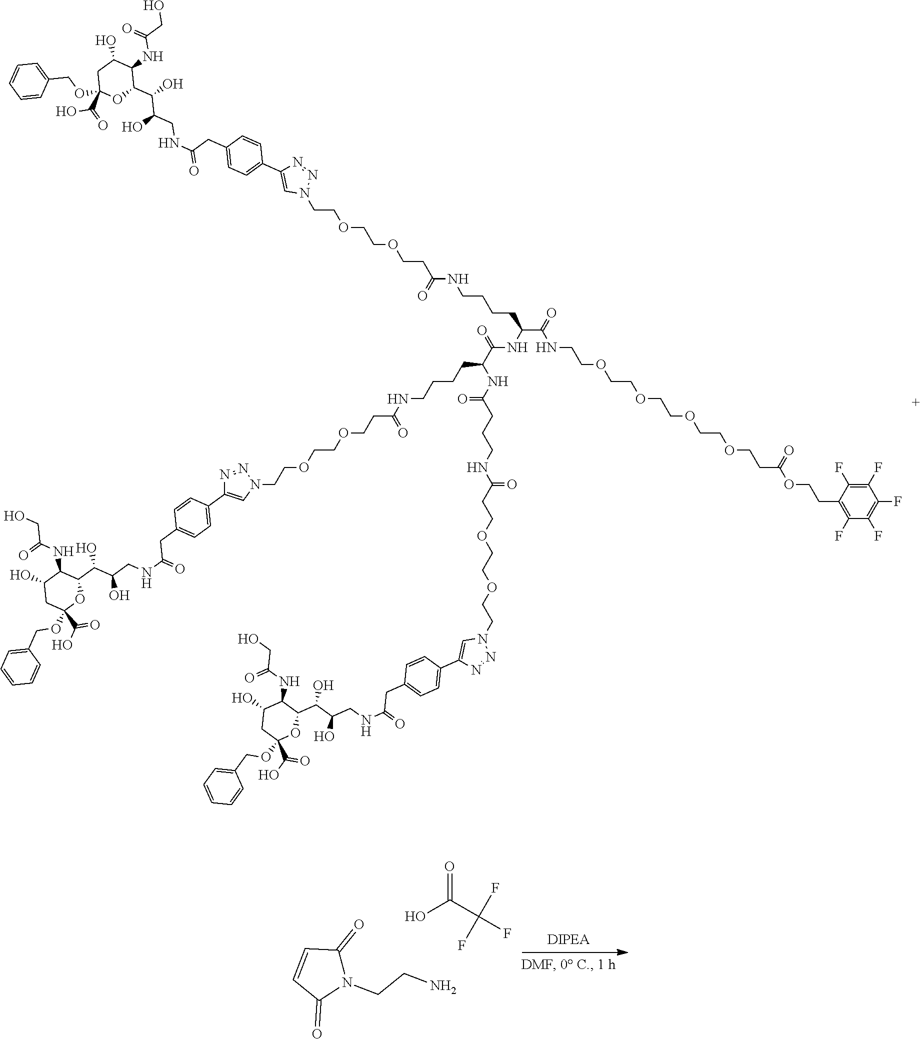 [SMILES: O=C(CCOCCOCCn1cc(-c2ccc(CC(=O)NC[C@@H](O)[C@@H](O)[C@@H]3O[C@@](OCc4ccccc4)(C(=O)O)C[C@H](O)[C@H]3NC(=O)CO)cc2)nn1)NCCCC[C@H](NC(=O)CCCNC(=O)CCOCCOCCn1cc(-c2ccc(CC(=O)NC[C@@H](O)[C@@H](O)[C@@H]3O[C@@](OCc4ccccc4)(C(=O)O)C[C@H](O)[C@H]3NC(=O)CO)cc2)nn1)C(=O)N[C@@H](CCCCNC(=O)CCOCCOCCn1cc(-c2ccc(CC(=O)NC[C@@H](O)[C@@H](O)[C@@H]3O[C@@](OCc4ccccc4)(C(=O)O)C[C@H](O)[C@H]3NC(=O)CO)cc2)nn1)C(=O)NCCOCCOCCOCCOCCC(=O)OCCc1c(F)c(F)c(F)c(F)c1F ; NCCN1C(=O)C=CC1=O ; O=C(O)C(F)(F)F]

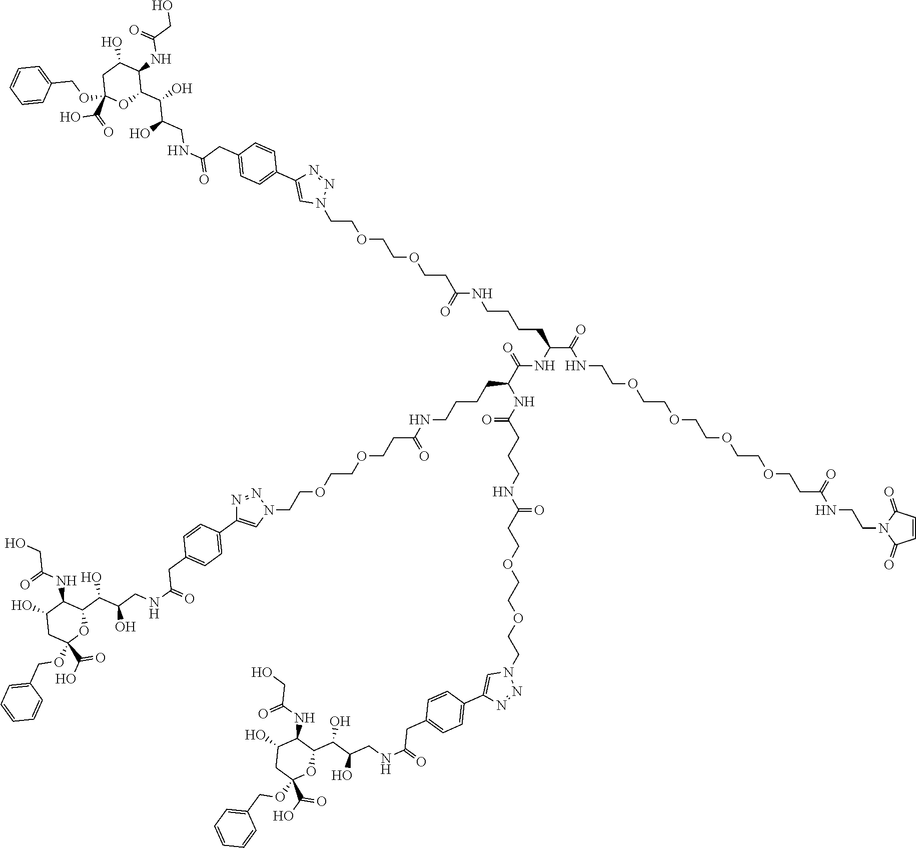 [SMILES: O=C(CCOCCOCCOCCOCCNC(=O)[C@H](CCCCNC(=O)CCOCCOCCn1cc(-c2ccc(CC(=O)NC[C@@H](O)[C@@H](O)[C@@H]3O[C@@](OCc4ccccc4)(C(=O)O)C[C@H](O)[C@H]3NC(=O)CO)cc2)nn1)NC(=O)[C@H](CCCCNC(=O)CCOCCOCCn1cc(-c2ccc(CC(=O)NC[C@@H](O)[C@@H](O)[C@@H]3O[C@@](OCc4ccccc4)(C(=O)O)C[C@H](O)[C@H]3NC(=O)CO)cc2)nn1)NC(=O)CCCNC(=O)CCOCCOCCn1cc(-c2ccc(CC(=O)NC[C@@H](O)[C@@H](O)[C@@H]3O[C@@](OCc4ccccc4)(C(=O)O)C[C@H](O)[C@H]3NC(=O)CO)cc2)nn1)NCCN1C(=O)C=CC1=O]

To a stirred solution of Cpd. 17 (29.3 mg, 9.77 umol) in anhydrous DMF (244 uL) was added n-(2-aminoethyl)maleimide trifluoroacetate salt (2.61 mg, 10.3 umol). The mixture was cooled down to 0° C. and stirred for 10 min. DIPEA (5.11 uL, 29.3 umol) was added and the mixture was stirred for 1 h at 0° C. The reaction mixture was filtered and purified by preparatory HPLC using acetonitrile and water (+0.05% TFA) as eluents. The fractions containing the desired product were combined and lyophilized to dryness to afford Cpd. 57 as a white solid. Yield: 23.7 mg, 82%; LCMS (ESI) m/z 1477 [M+2H]²⁺.

### Example 2: Binding Properties of Compounds

### Purpose

To characterize the binding affinities of the synthetic Siglec compounds by surface plasmon resonance (SPR).

### Materials and Methods

The following proteins were used for CD22 ligand binding: biotinylated human siglec-2/CD22 protein-His-Avitag (Acro Biosystems, Cat #SI2-H82E3), biotinylated mouse siglec-2-Fc-Avi (in-house production), and recombinant cynomolgus siglec-2/CD22 His-tag (R&D systems, Cat #9864-SL-050). Prior to immobilization, CD22 proteins were treated with SialoExo (Genovis, Cat #G1-SM1-020) for 4 hours at 37° C. to remove native sialic acid residues.

Capture of biotinylated CD22 constructs was performed using a Cytiva SA chip (BR100398) on a Biacore 8K instrument. Human siglec 2 was immobilized on channels 1-4 and mouse siglec 2 on channels 5-8 on the active flow cell (2) and the reference flow cell (1) was immobilized with biotinylated human FGFR2 Fc Avi, an unrelated protein, to account for any nonspecific binding. Human siglec 2 and Mouse siglec 2 were diluted in running buffer to 20 μg/mL and injected on the active flow cell (2) for 300 seconds at 5 μL/min, yielding about 3500 RU of captured constructs.

Surface preparation for cyno CD22 protein consisted of amine coupling to Cytiva CM5 chip (Cytiva, Cat #BR100530) using the Cytiva amine coupling kit Type 2 (Cytiva, Cat #BR100633). Following the standard amine coupling protocols, EDC and NHS from the kit were mixed 1:1 and injected over flow cells 1 and 2. Cyno CD22 was then immobilized by injecting 10 μg/mL in Sodium Acetate, pH 5.5 onto flow cell (2) for 420 seconds at 10 uL/min, yielding approximately 2500 RU of captured protein. Any active carboxy groups were then capped with 1M Ethanolamine, pH 8.5 for 420 s on flow cells 1 and 2.

Binding experiments of siglec-ligand conjugated proteins or Siglec ligand compounds were performed on the surfaces prepared above in 10 mM HEPES, 150 mM NaCl, 0.05% Tween-20, pH 7.5, 1% DMSO as the running buffer. Compounds were serially diluted in running buffer from 50 μM to 97.6 nM and injected over both the reference and active flow cells for 90 seconds at 30 μL/min. The compounds were then allowed to dissociate from the surface for 120 seconds. No regeneration was required as the compounds completely dissociated from the surface after 30 seconds. Solvent correction was performed using 0.5, 1, 1.5, and 2% DMSO spike into running buffer without DMSO. Data was analyzed using steady-state analysis in Biacore Insight Evaluation Software, version 3.0.

The biochemical properties of the compounds were measured, as shown in the table below. Kd>10 μM was “+”, 1 μM<=Kd<=10 μM was “++”, and Kd<1 μM was +++.

| | Mouse CD22 | Human CD22 | Cyno CD22 |
|---|---|---|---|
| Compound | affinity | affinity | affinity |
| 1 | +++ | +++ | ++ |
| 2 | ++ | ++ | ++ |
| 3 | ++ | +++ | +++ |
| 4 | ++ | ++ | ++ |
| 5 | ++ | ++ | ++ |
| 6 | ++ | ++ | + |
| 7 | ++ | ++ | ++ |
| 8 | +++ | +++ | ++ |
| 9 | ++ | ++ | ++ |
| 10 | ++ | ++ | +++ |
| 11 | ++ | +++ | +++ |
| 12 | +++ | +++ | +++ |
| 13 | ++ | ++ | ++ |
| 14 | +++ | ++ | ++ |
| 15 | ++ | ++ | ++ |
| 16 | +++ | +++ | +++ |
| 18 | ++ | ++ | ++ |
| 19 | ++ | ++ | ++ |
| 20 | ++ | ++ | ++ |
| 21 | + | ++ | ++ |
| 22 | ++ | + | ++ |
| 25 | ++ | ++ | ++ |
| 26 | +++ | +++ | +++ |
| 27 | +++ | +++ | +++ |
| 28 | +++ | +++ | +++ |
| 29 | +++ | +++ | +++ |
| 52 | ++ | ++ | ++ |
| 53 | ++ | +++ | +++ |
| 54 | | | |
| 55 | ++ | +++ | ++ |
| 56 | | | |
| 57 | | | |

### Example 3: Analysis of Synthetic CD22 Binding Specificity Across Various Siglec Receptor Family Members

### Purpose

The purpose of this study is to examine Siglec ligand specificity for Siglec-2 in comparison to other Siglec family members. A competitive bead-based flow cytometry assay is developed to assess selectivity of CD22 ligands for Siglec proteins. Briefly, the assay employs a streptavidin bead coated with biotinylated CD22 ligand. The beads are then incubated with human Siglec-2-AF647 at a low concentration and competitive unlabeled Siglec proteins are added. A decrease in Siglec-2-AF647 indicates competitive binding of the CD22 ligand beads with the unlabeled Siglec protein.

### Materials and Methods

The following recombinant Siglec proteins are obtained from commercial sources: human Siglec-2-Fc fusion (R&D systems, Cat. No. 1968-SL-050), human Siglec-3-Fc fusion (Acro Biosystems, Cat. No. CD3-H₅₂₅₇), human Siglec-4a-Fc fusion (Acro Biosystems, Cat. No. MAG-H₅₂₅₄), human Siglec-15-Fc fusion (Acro Biosystems, Cat. No. SG5-H5253), Mouse Siglec-1-Fc fusion (R&D systems, Cat. No. 5610-SL-50), and human Siglec-2-Fc fusion labeled with AF647 (R&D systems, Cat. No. AFR1968-020).

Dynabeads M-280 Streptavidin-coated beads (20 μl of 6.7×10⁸ beads per mL, Thermo Scientific, Cat. No. 11205D) are washed with (−/−) phosphate buffered saline containing 1% bovine serum albumin and 0.1% sodium azide (FACS buffer), centrifuged beads at 350 g for 1 min and aspirated the supernatant. Beads are resuspended in 200 μL of FACS buffer and then incubated with biotinylated-CD22 ligand (5 μM) overnight at 4° C. Separate beads are coated for each CD22 ligand tested.

In a 96-well U-bottom plate, human Siglec-2 Fe AF647 at a fixed concentration of 10 nM is added to the following concentrations of Siglec proteins: human Siglec 2 Fe protein (no AF647) at 200 nM; mouse Siglec-1 Fe at 150 nM; human Siglec-3-Fc at 200 nM; human Siglec-4-Fc at 200 nM; human Siglec-15-Fc at 200 nM. CD22 ligand-beads are washed and resuspended in FACS buffer.

Approximately 33,500 CD22 ligand-beads are added per well and mixed by pipette. After 60 min at room temperature, protected from light, samples are analyzed by flow cytometry at 150 events per sec. Flow cytometry is performed on a Biorad ZE5 analyzer and data is processed using FlowJo LLC. The single bead populations are gated out to obtain the median fluorescence intensity (MFI) of AF647, which indicates binding of the human Siglec-2 Fe-AF647 to the surface of the CD22 ligand-conjugated bead. MFI values are used to generate binding curves (variable slope four parameters) in GraphPad Prism.

### Results and Conclusions

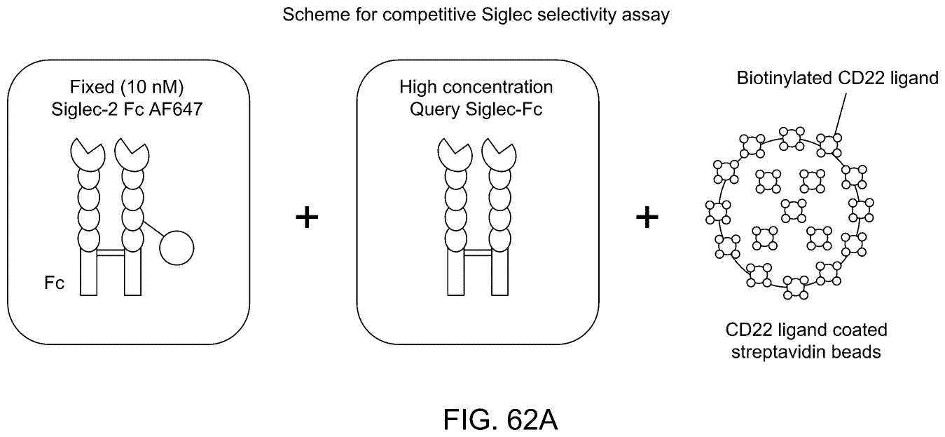

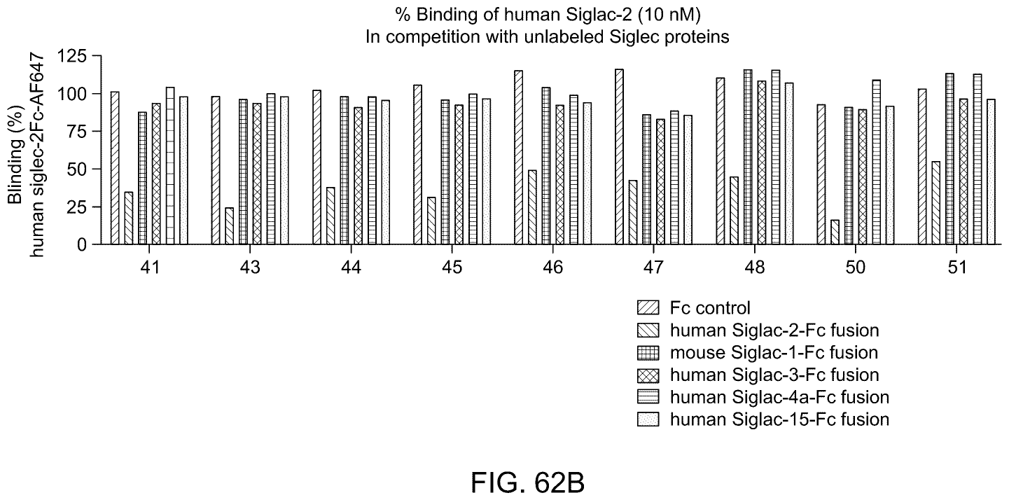

FIG. 62A shows a scheme for the competitive Siglec assay where a fixed concentration of human Siglec-2-Fe-AF647 is incubated with CD22 ligand-bead and a competitor Siglec-Fc fusion protein. FIG. 62B shows the percent binding of the AF647-labeled human Siglec-2 high concentrations of competitive Siglec protein (human Siglec-2-Fc at 200 nM; mouse Siglec-1-Fc at 150 nM; human Siglec-3-Fc at 200 nM; human Siglec-4-Fc at 200 nM; human Siglec-15-Fc at 200 nM) to the various CD22 ligand-beads. Each of the CD22 ligands examined demonstrated a decrease in binding of the human Siglec-2-Fe-AF647 where a high concentration of unlabeled human Siglec-2-Fc fusion is present, indicating that CD22-ligands bind human Siglec-2-Fc fusion.

Human Siglec 3, 4a, 15, and mouse Siglec-1 are also tested for competitive displacement of AF647-label Siglec-2. Those Siglecs showed no significant levels of AF647-labeled Siglec-2 signal decreases, indicating that they do not bind to the CD22 ligands. An isotype control antibody is also used as a competitor protein and confirmed that binding is specific for Siglec and not Fe fusion (labeled Fe control). Overall, this panel of CD22 ligands shows high specificity for Siglec-2 and does not show binding to other Siglec proteins tested.

### Example 4: Antibody (Anti-Mouse-IgD Antibody, Anti-Human-IgM Antibody, and Adalimumab) Expression, Purification, Conjugation, and Analytics

### Materials and Methods

For antibody expression, the ExpiFeetamine 293 Transfection kit (Life Technologies, A14524) was used to transfect suspension Expi293F cells (Life Technologies, A14527) with Heavy Chain and Light Chain plasmids (pTT5-based) at a 1:1 ratio. Media was harvested 3-6 days post-transfection by centrifugation and filtered using 0.2 μm PES vacuum sterile single-use filter unit (ThermoScientific, 5670020).

Purification was performed with 1.5 mL MabSelect Sure resin (Cytiva/GE Cat #: 17-5438-03) for each 250 mL culture supernatant. Briefly, each column was equilibrated with PBS pH 7.2 and loaded with culture supernatant. After the loading step, the column was washed with PBS pH 7.2 and eluted with 10 mL IgG Elution buffer (Thermo Scientific Ref 21004). The pH of the elution pool was adjusted with 1 mL 1 M Sodium Phosphate pH 6.5 for each 10 mL elution pool. Finally, buffer exchange was performed with PBS pH 7.2 using a 30 kDa Amicon Ultra-15 Centrifugal Filter Unit.

Analysis of endotoxin content was performed using the Charles River Endosafe PTS 0.01-1 EU/ml detection. Size exclusion chromatography was performed on an Agilent Chemstation HPLC-SEC with a Sepax-Zenix SEC-300, 200 mm×7.8 mm ID, 3 uM column. Capillary gel electrophoresis (cGE) was performed on a Caliper LabChip GXII Protein 200 with the Perkin Elmer Chip (Cat #760499). LC-MS analysis was performed on SciEX LC 5600+, ExionLC AD, Analyst TF 1.8.1 with an Agilent AdvanceBio Desalting-RP, Column 1000 A, 10 um.

### Protein-Siglec Ligand Conjugation to Lysines

Pentafluorophenyl (PFP) conjugatable siglec Ligand linker was added to reaction mixtures at a molar ratio of 4-30 times above protein based on desired degree of labeling in the presence of 10% v/v of 50 mM Sodium Tetraborate pH 8.5 and 10% v/v DMSO. Reactions were incubated for 3 hours at 25° C. After the 3 h incubation period, 10% v/v of 1 M Tris-HCl pH 8.0 was added to quench the unreacted linker-payload. Neutralized reactions were then allowed to incubate at 25° C. for 15 min.

### Protein-Siglec Ligand Conjugation to Site-Specific Cysteines

One mutation site was employed in the heavy chain region of the antibodies to incorporate new cysteine sites, L443C. The cysteine-engineered protein was diluted in a solution containing 1 mM diethylenetriaminepentaacetic acid (DTPA) and 100 mM HEPES at pH 7.0. To this mixture, tris(2-carboxyethyl)phosphine (TCEP) was added in a 20-fold molar excess above protein. The resulting mixture was incubated at 37° C. for 2 h. After incubation, excess TCEP was removed using a desalting column equilibrated with PBS at pH 7.2.

The reduced protein was then re-oxidized in a solution containing 100 mM HEPES at pH 7.0, 1 mM DTPA, and 2 mM dehydroascorbic acid (DHA), and incubated at 4° C. for 16 h. Maleimide-conjugatable Siglec Ligand linkers in 10% v/v dimethylacetamide (DMA) were added to the reaction mixtures at a molar ratio of 10-60 times above protein, based on the desired degree of labeling. Reactions were incubated at 25° C. for 2 h. After the incubation period, 10% v/v of 100 mM reduced glutathione at pH 7.0 was added to quench any unreacted maleimide linkers. The neutralized reactions were incubated at 25° C. for an additional 15 min.

### Protein Siglec-Ligand Conjugate Purification

Quenched conjugation reactions are purified by preparative size exclusion chromatography at 4° C. using either Superdex 200 Increase 10/300 GL or HiLoad 16/600 Superdex 200 pg at a flow rate of 0.75 mL/min, with PBS pH 7.2

### Analysis of Protein-Siglec Ligand Conjugates

The “LDR”, or Ligand-to-Drug ratio, was measured for each conjugate preparation by LC/MS, by evaluating the relative abundances of species varying in the degree of conjugation, as described here. Random conjugation methods (to lysine/amines in this case) result in a mixture of species varying in the degree of conjugation per adalimumab species. Such a series of molecular species can be represented as:

$${\sum }_{i = 1}^{m} {[ {X}_{n} L ]}_{i} Y$$

In this statement, each biotherapeutic, Y (adalimumab, in this case), is covalently bound to a Siglec Ligand as defined by XₙL, where X is a sialic acid species of valency, n, with a Siglec Ligand-to-biotherapeutic ratio that varies between 1 and m. All species have the same Sialic Acid valency, n (monovalent, bivalent, or trivalent).

As a total measure of the degree of conjugation in such an ensemble of species with varying degrees of conjugation, the LDR can be defined as follows: LDR is a weighted average of the individual Siglec ligand-to-biotherapeutic ratios (integer value m) in a mixture of species varying in said ratio, and Pᵢ(0≤Pᵢ≤1, with Σᵢ₌₁ⁿ Pᵢ=1) representing the fractional abundance of each species in the mixture:

$$LDR = {\sum }_{i = 1}^{m} {P}_{i} {m}_{i}$$

Protein-siglec ligand conjugates were analyzed using the following parameters.

### LC-MS

Mass spectrometer: SciEX LC 5600+, ExionLC AD, Analyst TF 1.8.1
HPLC: Agilent AdvanceBio Desalting-RP, Column 1000 A, 10 μm
2.1 mm×12.5 mm, flow rate 400 ul/min, Sample load: 5 ug
Buffer A: Water+0.1% Formic Acid
Buffer B: Acetonitrile+0.1% Formic Acid

### Analytical SEC

Superose 6 Increase 10/300 GL
Injection Volume: 25 uL at 1 mg/mL of sample.
Buffer: 50 mM Sodium Phosphate+400 mM Sodium Perchlorate, pH 6.2
Flow rate: 0.7 mL/min
Run time 40 min at 25° C.

### Capillary Gel Electrophoresis

Caliper LabChip GXII Protein 200

### Endotoxin Measurement

Charles River Endosafe PTS Cartridge 0.01-1 EU/ml
Lonza Pyrogene C Endpoint Endotoxin Assay

### Results and Conclusions

The results below show a summary of conjugates that were described herein.

| | Conjugation | | | Compound | Conjugate |
|---|---|---|---|---|---|
| Protein | site | LDR | Conjugation type | ID | ID |
| anti-mouse-IgD | N/A | 6.7 | Pentafluorophenyl coupling to lysine | 17 | NHC017 |
| anti-mouse-IgD | N/A | 5.5 | Pentafluorophenyl coupling to lysine | 23 | NHC023 |
| anti-human-IgM | N/A | 4.6 | Pentafluorophenyl coupling to lysine | 30 | NHC901 |
| anti-human-IgM | N/A | 4.8 | Pentafluorophenyl coupling to lysine | 31 | NHC902 |
| anti-human-IgM | N/A | 5.5 | Pentafluorophenyl coupling to lysine | 32 | NHC903 |
| anti-human-IgM | N/A | 5.4 | Pentafluorophenyl coupling to lysine | 33 | NHC904 |
| anti-human-IgM | N/A | 4.8 | Pentafluorophenyl coupling to lysine | 34 | NHC905 |
| anti-human-IgM | N/A | 5 | Pentafluorophenyl coupling to lysine | 35 | NHC906 |
| anti-human-IgM | N/A | 4.4 | Pentafluorophenyl coupling to lysine | 36 | NHC907 |
| anti-human-IgM | N/A | 4.8 | Pentafluorophenyl coupling to lysine | 37 | NHC908 |
| anti-human-IgM | N/A | 4.3 | Pentafluorophenyl coupling to lysine | 38 | NHC909 |
| anti-human-IgM | N/A | 4.1 | Pentafluorophenyl coupling to lysine | 39 | NHC910 |
| anti-human-IgM | N/A | 4.8 | Pentafluorophenyl coupling to lysine | 40 | NHC911 |
| Adalimumab | HC-L443C | 2 | Maleimide coupling to cysteine | 56 | NHC912 |

All conjugates were purified to homogeneity for oligomeric species, with the intended oligomeric structure (e.g., monomer, dimer, trimer) being purified by preparative size exclusion chromatography. All control protein preparations, and conjugate preparations were >99% pure by analytical SEC.

### Example 5: Suppression of B Cell Receptor-Mediated Activation of Mouse Primary B Cells by Siglec Ligand-Anti-IgD Antibody Conjugates

### Purpose and Introduction

The purpose of this experiment was to test for suppressive effects on mouse B cell activation of B cell receptor (BCR) agonist IgG-siglec ligand conjugates.

The platform technology described rests on the premise that activation of B cells through their clonotypic B cell receptor can be suppressed through physical recruitment of the CD22/siglec-2 inhibitory coreceptor to co-engaged B cell receptor. CD22 recruited to the B cell receptor is phosphorylated on its ITIM cytoplasmic motif tyrosines by virtue of its proximity to the high local protein kinase activity at the B cell receptor. Phosphorylated CD22 then recruits phosphatases, such as SHP-1 and SHP-2, to the cell surface, in proximity of the B cell activation complex. Such elevated local phosphatase activity dephosphorylates components of the B cell activation complex necessary for B cell activation, thus shutting down responses to B cell receptor engagement. Under normal circumstances, the siglec-2 immunoinhibitory mechanism acts as a check on aberrant B cell activation, safeguarding against autoreactive antibody production, hyperinflammation, and autoimmunity. The described platform technology exploits this natural phenomenon to cloak foreign proteins as self, dampening B cell activation only on naïve B cell clones that are specific for the given foreign protein and thus blocking immunoglobulin production against the foreign protein, while leaving B cell responses to other antigens intact.

The high diversity of primary B cell populations, and high diversity of B cell receptor sequences and clones (as high as 10¹² per human), presents a challenge for studying BCR agonism in vitro with a single, well-defined BCR antigen. For this reason, pan-BCR activators, such as anti-IgD or anti-IgM antibodies, that can bind, crosslink, and activate the BCR—regardless of B cell/BCR clonality—are used to evaluate BCR activation in vitro. In the experiments described in this example, an anti-mouse IgD monoclonal antibody is used either in parental IgG form or as IgG-siglec-ligand conjugates to study the effects of siglec-2-B cell receptor co-engagement on B cell activation.

To control for impacts of anti-IgD conjugation on BCR binding potency, competition binding assays were used to assess binding activity of Siglec Ligand conjugates and ensure that apparent suppressive effects were due to siglec-2×BCR co-engagement and not a general damaging of anti-IgD for receptor binding.

### Materials and Methods

Anti-mouse-IgD and Anti-mouse-IgD-Siglec Ligand test articles were prepared as described in Example 4.

Splenocytes from C57BL/6 mice were harvested into single cell suspension, subjected to red cell lysis using ACK buffer, and plated at a concentration of 200,000 cells per well in round bottom 96 well plates in complete RPMI media. Cells were stimulated by the addition of increasing concentration of anti-mouse IgD or Siglec ligand-conjugated anti-mouse IgD and incubated for 3 hours at 37° C., 5% CO₂. Cell activation was assessed by flow cytometry.

To measure B cell activation following the described stimulation, cells were washed twice by centrifugation at 350 g for 5 minutes and adding staining buffer (1% bovine serum albumin/0.1% sodium azide/1×phosphate buffered saline). Cells were then resuspended in staining buffer and incubated with Fc-block (Biolegend) for five minutes before the addition of anti-CD45, anti-CD19, anti-CD69, and anti-CD86 antibodies (BD Biosciences, Biolegend, Fisher). Cells were then incubated in the dark for 30 minutes at room temperature. Cells were then washed three times with staining buffer and analyzed on ZE5 (BioRad). Data analysis was performed using FlowJo (v10.8.0) software.

### General Gating Strategy:

1. Lymphocyte gate set on FSC 488 10-A/SSC 488-10-A
2. Single cells gate set on FSC 488 10-H/FSC 488-10-A
3. CD45+ gate set on CD45-FITC+ population using CD45-FITC-A/SSC 488-10-A
4. % CD69+ gate set on CD69-Alexa647-A/CD19-BV421-A
5. Mean Fluorescence Intensity (MFI) gate is set on CD19+ cells using CD69-Alexa647-A/CD19-BV421-A

### Results and Conclusions

FIGS. 62A and 62B depict an in vitro primary mouse B cell activation assay testing for the importance of CD22 engagement for Siglec Ligand-conjugate-mediated suppression of B cell receptor activation. Mouse primary B cells were treated with either a B cell receptor agonizing anti-IgD antibody or various anti-IgD-Siglec Ligand conjugates. In this case, conjugates (NHC017 and NHC023) carried derivatives of molecules 17 and 23, covalently coupled to anti-IgD antibody using lysine chemistry as described in Example 4. The anti-IgD antibodies were conjugated at an LDR of 6.7 and 5.5 sites respectively, where the NHC017 as a trimer ligand would contain 20 Siglec binding sites. A competition binding assay was used to confirm that anti-IgD binding was maintained. No significant changes in binding affinity were observed with the anti-IgD conjugates with the high degree of labeling (data not shown), indicating that any suppression observed was not due to inhibition of IgD binding.

Where parental anti-IgD antibody induces a strong, concentration-dependent increase in % CD69-positive cells and in CD69 MFI, Siglec Ligand-anti-IgD conjugates show varying degrees of suppression. Moreover, the degree of ADA suppression correlates well with the number of Siglec ligands conjugated to the anti-IgM, with high numbers of Siglec ligands correlating with higher suppression.

### Example 6: Suppression of B Cell Receptor-Mediated Activation of Human Primary B Cells by Siglec Ligand-Anti-IgM Antibody Conjugates

### Purpose

The purpose of this experiment was to test for suppressive effects on human B cell activation with B cell receptor agonist IgG-Siglec Ligand conjugates. This experiment is analogous to the one described in Example 5, with the focus here on primary human, PBMC-derived B cells, rather than the primary mouse splenocytes.

### Materials and Methods

Anti-human-IgM and Anti-human-IgM-Siglec Ligand test articles were prepared as described in Example 4. In this case, the anti-IgM constructs were conjugated at lysine using a reactive pentafluorophenyl Siglec ligand.

Human PBMCs (StemExpress) were plated at a concentration of 200,000 cells per well in round bottom 96 well plates in complete RPMI media. Cells were stimulated for 18 hours by the addition of increasing concentration of anti-human IgM or Siglec ligand-conjugated anti-human IgM. B cell activation was assessed by flow cytometry.

To measure B cell activation following the described stimulation, cells were washed twice by spinning cells at 1200 rpm for 5 minutes and rinsing with PBS. Cells were resuspended in staining buffer (1% bovine serum albumin/0.1% sodium azide/1× phosphate buffered saline) and incubated with Fc-block (BD Biosciences) for five minutes before the addition of anti-CD45, anti-CD19, anti-CD69, and anti-CD86 antibodies (BD Biosciences, Biolegend, Fisher). Cells were incubated in the dark for an additional 30 minutes at room temperature, then washed three times with staining buffer and then analyzed on ZE5 (BioRad). Data analysis was performed using FlowJo (v10.8.0) software.

Competition of Siglec ligand-conjugated anti-human IgM binding was carried out on human PBMCs from the same donors used in assays described above. For this assay, cells were seeded at a concentration of 200,000 cells per well in round bottom 96 well plates in complete RPMI media. Cells were subsequently incubated with human Fc-block (BD Biosciences) for five minutes. Following this incubation period, 2.4 nM anti-human-IgM-AlexaFluor647 was added to the cells along with RPMI alone or an increasing titration of non-fluorescently labeled Siglec ligand-conjugated anti-human IgM and anti-CD19. Cells were incubated at 4° C. for 30 minutes in the dark. After incubation, cells were washed twice by centrifugation with staining buffer (1% bovine serum albumin/0.1% sodium azide/1×phosphate buffered saline) and antibody binding was analyzed by flow cytometry (ZE5, BioRad) by determination of the mean fluorescence intensity (MFI) of at least 5,000 cells. The gating strategy was similar to the gating strategy described in Example 5.

### Results and Conclusions

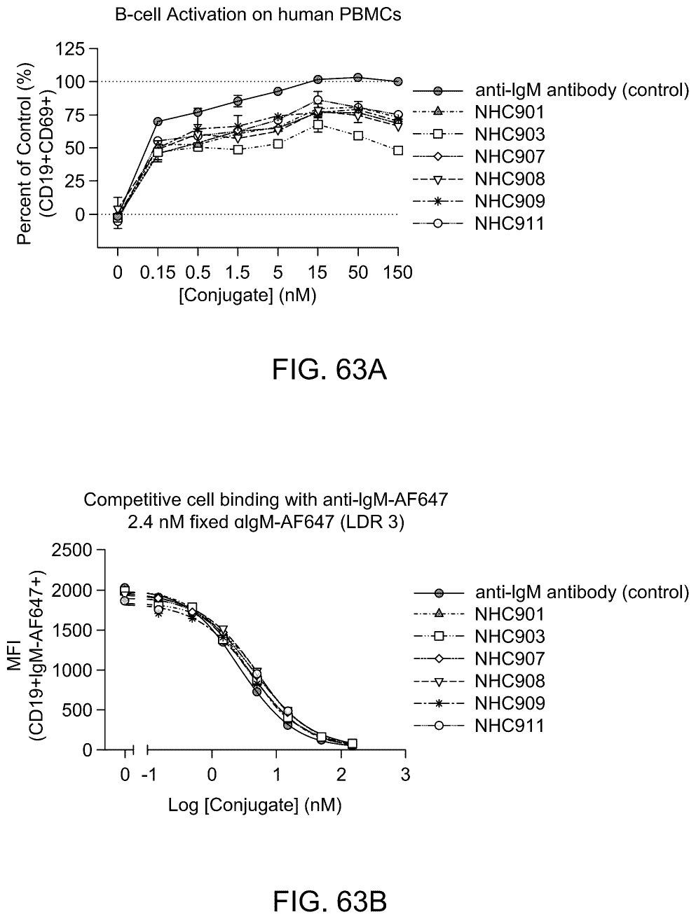

FIG. 63A shows representative B cell stimulatory activities of anti-IgM and anti-IgM-CD22 ligand conjugates in dose titration experiments with an activation readout of CD69 upregulation. CD69 levels on the different treatment groups are evaluated through the percentage of cells that are CD69-positive. The table below shows a summary of all the compounds tested in the assay with the percentage of cells that are CD69-positive at 150 nM normalized to control anti-IgM antibody.

| Conjugate | % CD69 at 150 nM |
|---|---|
| NHC901 | 69.6 |
| NHC902 | 67.6 |
| NHC903 | 48.2 |
| NHC904 | 97.7 |
| NHC905 | 71.4 |
| NHC906 | 75.8 |
| NHC907 | 68.1 |
| NHC908 | 65.7 |
| NHC909 | 71.9 |
| NHC910 | 85.0 |
| NHC911 | 75.3 |

Where parental anti-IgM IgG induces a strong, concentration-dependent increase in % CD69-positive cells and in CD69 MFI, Siglec Ligand-anti-IgM conjugates show strong suppressed activation. Moreover, as shown in FIG. 63B, there is no interference of the conjugates with IgM binding, so the suppression is not a reflection of decreased IgM binding. Importantly, these data show translation of the suppressive effect of Siglec-2 Ligand conjugates between a primary human B cell system and those shown in a primary mouse B cell system.

### Example 7: In-Vivo Suppression of Anti-Drug Antibody in Mice Treated with Siglec Ligand-Adalimumab Conjugates

### Purpose

The purpose of this experiment was to test for suppression of immunogenicity in mice dosed with adalimumab-Siglec-2 ligand conjugates. Parental adalimumab hIgG1 is highly immunogenic in mice, with a strong immunoglobulin response after a single dose. This example set out to corroborate the in vitro B cell suppressive effects shown in Examples 5 and 6 with in vivo assessment of effects on immunogenicity for Siglec Ligand conjugates with different proteins.

### Materials and Methods

Adalimumab and Adalimumab-Siglec Ligand conjugates were prepared as described in Example 4. The Adalimumab-Siglec ligand was conjugated by maleimide coupling to engineered cysteine residue L443C in the heavy chain constant region.

To evaluate the production of antibodies specific to adalimumab and/or adalimumab-Siglec Ligand conjugates, C57BL/6 mice were immunized through subcutaneous injection with adalimumab or CD22 ligand-conjugated adalimumab. On study day −1, animals were randomized into treatment groups based on body weight. On study day 0, animals were bled for baseline serum and then injected subcutaneously with adalimumab or the adalimumab-Siglec Ligand conjugate. The individual antigens were prepared by making antigen solution in sterile PBS pH 7.4. Animals were then injected subcutaneously with 0.2 mL total of the 0.5 mg/mL antigen where 0.1 mL was injected on each side of the rear flank near base of tail. The total dose based on a 25 g mouse was 4 mg/kg. Animals were bled via the retro-orbital sinus weekly (days 6, 13, 20) throughout the study under inhaled isoflurane anesthesia. On study day 28, animals were anesthetized with inhaled isoflurane anesthesia and then bled via cardiac puncture and then sacrificed by cervical dislocation. Whole blood was collected into Microvette EDTA capillary collection tubes (Sarstedt Inc) and then further processed following the manufacturer's instructions for serum collection. Samples were stored at −80° C. until analysis was performed.

ADA assays were performed on 96-well assay plates (Nunc Plates, Black 96-Well Immuno Plates, Thermo Scientific, Cat. No. 437111) coated with antigen, as follows. A mixture of adalimumab and adalimumab conjugate was coated at 5 μg/ml of each, with 100 μL/well. All coated antigens were diluted in PBS pH 7.2 and incubated overnight at 4° C. The following day, plate coating solution was removed, and plates were blocked with 200 μL/well of 3% BSA, 20 μM EDTA, 0.1% Tween-20 in PBS for 1 hour at room temperature. Serum samples were diluted 1:185 in 3% BSA, 20 μM EDTA, 0.1% Tween-20 in PBS and added in three-fold serial dilutions. Plates were incubated 1 hour at room temperature, then washed with PBS buffer with 0.05% Tween-20. After washing, 100 μL of 1:2500 diluted Donkey Anti-Mouse IgG(H+L)-HRP (SouthernBiotech, Cat. No. 6411-05) was added and incubated for 1 hour at room temperature. After washing the assay plates, 100 μL of QuantaBlu Substrate Solution (Thermo Scientific, Cat. No. 15169) was added to each well and incubated for 15 minutes. The excitation and emission settings for the QuantaBlu Fluorogenic Peroxide Substrates are 325 nm and 420 nm and the relative florescence units were measured using a SpectraMax plate reader. Serum dilution curves were generated for days 7, 14, 21, and 28. The titer cut point was determined by calculating the mean of the naïve, untreated mice and adding twice the standard deviation of the untreated samples.

Pharmacokinetic monitoring was also carried out in the same C57BL/6 mice following a single subcutaneous administration of adalimumab or the SigL-conjugated adalimumab antibodies. Animals were bled via the retro-orbital sinus throughout the study under inhaled isoflurane anesthesia at 1 h, 6 h, 1-day, 2-days, 6-days, 13-days, 20 days and 27-days post test-article administration. Whole blood was collected into Microvette K2EDTA capillary collection tube (Sarstedt Inc) and then further processed following manufactures instructions to collect plasma. Levels of adalimumab and SigL-conjugated adalimumab in plasma samples were measured using an AlphaLISA human IgG assay (Perkin Elmer) following manufacturer's protocols.

### Results and Conclusions

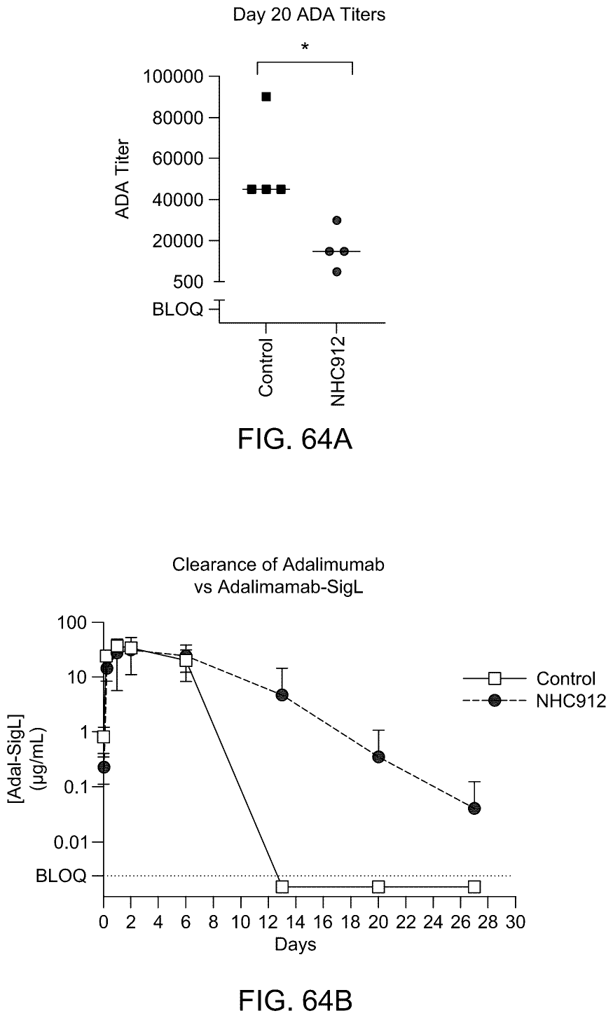

FIG. 64A shows the ADA titers of mice dosed at 4 mg/kg at day 20. Adalimumab clearly shows high ADA titers on day 20. In contrast, the CD22-ligand Adalimumab conjugate shows significantly lower ADA titers. These results for suppression of immunogenicity in mice correlate with the in vitro B cell activation results in Examples 5 and 6; decoration of an immunogenic antibody with potentiated CD22 ligand is sufficient to strongly suppress immunogenicity.

FIG. 64B depicts analysis of serum pharmacokinetics for adalimumab and CD22-ligand-adalimumab conjugate in mice. Clearly, the control adalimumab displays significantly increased clearance, falling below the limit of quantitation (BLOQ) at day 13 in FIG. 64B. This is likely due to the high ADA titers observed with adalimumab, as shown in FIG. 64A.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112(f) is not invoked.

1. A siglec ligand of formula (I): 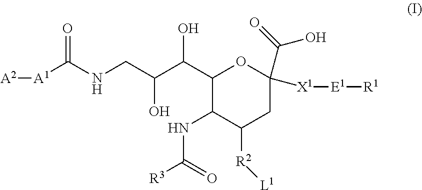
wherein:
A¹ is absent or —(CH₂)ₙ—, wherein n is an integer ranging from 1 to 10;
A² is alkyl, aryl, heteroaryl, cycloalkyl, or a substituted version thereof;
R¹ is H, alkyl, alkoxy, carboxyl, ester, amine, aryl, heteroaryl, cycloalkyl, heterocycle, carbohydrate, phosphate, squaramide, or a substituted version thereof;
R² is O, S, —NR⁴ R⁵, or —CR⁴ R⁵; wherein R⁴ and R⁵ are each independently selected from the group consisting of H, alkyl, substituted alkyl, alkenyl, substituted alkenyl, alkynyl, substituted alkynyl, cycloalkyl, substituted cycloalkyl, heterocyclyl, substituted heterocyclyl, aryl, substituted aryl, heteroaryl, and substituted heteroaryl;
R³ is OR^{3′}, NHR^{3′}, alkyl, —CH₂OR^{3′}, —CH₂NHR^{3′}, wherein R^{3′} is H, alkyl, aryl, heteroaryl, cycloalkyl, or a substituted version thereof;
X¹ is O, S, —CH₂—, —CH(OH)—, or triazole;
E¹ is absent, —(CH₂CH₂O)_{g}— or —(CH₂)_{g}—, wherein g is an integer ranging from 1 to 10;
L¹ is a linker;
a salt thereof, or a stereoisomer thereof.

2. A siglec ligand of formula (II): 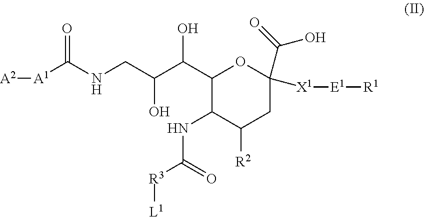
wherein:
A¹ is absent or —(CH₂)ₙ—, wherein n is an integer ranging from 1 to 10;
A² is alkyl, aryl, heteroaryl, cycloalkyl, or a substituted version thereof;
R¹ is H, alkyl, alkoxy, carboxyl, ester, amine, aryl, heteroaryl, cycloalkyl, heterocycle, carbohydrate, phosphate, squaramide, or a substituted version thereof;
R² is OH;
R³ is —O—, —NH—, —CH₂—, —CH₂O—, or —CH₂NH—;
X¹ is O, S, —CH₂—, —CH(OH)—, or triazole;
E¹ is absent, —(CH₂CH₂O)_{g}— or —(CH₂)_{g}—, wherein g is an integer ranging from 1 to 10;
L¹ is a linker;
a salt thereof, or a stereoisomer thereof.

3. A siglec ligand of formula (III): 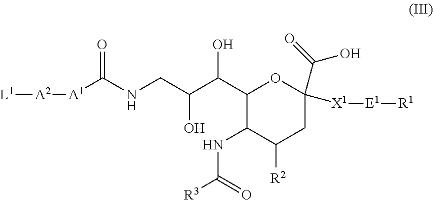
wherein:
A¹ is absent or —(CH₂)ₙ—, wherein n is an integer ranging from 1 to 10;
A² is alkyl, aryl, heteroaryl, cycloalkyl, or a substituted version thereof;
R¹ is H, alkyl, alkoxy, carboxyl, ester, amine, aryl, heteroaryl, cycloalkyl, heterocycle, carbohydrate, phosphate, squaramide, or a substituted version thereof;
R² is OH;
R³ is OR^{3′}, NHR^{3′}, alkyl, —CH₂OR^{3′}, —CH₂NHR^{3′}, wherein R^{3′} is H, alkyl, aryl, heteroaryl, cycloalkyl, or a substituted version thereof;
X¹ is O, S, —CH₂—, —CH(OH)—, or triazole;
E¹ is absent, —(CH₂CH₂O)_{g}— or —(CH₂)_{g}—, wherein g is an integer ranging from 1 to 10;
L¹ is a linker;
a salt thereof, or a stereoisomer thereof.

4. The siglec ligand of claim 1, wherein the linker L¹ is represented by —(X²)ₘ—Z, wherein:
each X² is independently selected from —(CH₂)ₕ—, —O—, —CH₂O—, —(CH₂CH₂O)ₕ—, —C(O)NH—, —C≡C—, and —CH₂C≡C—, triazole, carboxyl, ester, or substituted version thereof, wherein m is 0 or an integer ranging from 1 to 10, and h is an integer ranging from 1 to 10; and
Z is a chemoselective functional group.

5. The siglec ligand of claim 4, wherein the chemoselective functional group is selected from thiol, maleimide, iodoacetamide, amine, carboxyl, ester, triazole, tetrazine, transcyclooctene, diene, dienophile, sulfonyl fluoride, hydroxyl, hydrazido, hydrazino, aldehyde, ketone, azido, alkyne, phosphine, epoxide, succinimide, and pentafluorophenyl (PFP) ester, or a substituted version thereof.

6. The siglec ligand of claim 4, wherein:
each X² is independently selected from —(CH₂)ₕ—, —O—, —CH₂O—, —(CH₂CH₂O)ₕ—, —C(O)NH—, or a substituted version thereof, wherein m is 0 or an integer ranging from 1 to 10, and h is an integer ranging from 1 to 10; and
Z is alkyne, azido, hydroxyl, carboxyl, maleimide, thiol, amine, or a pentafluorophenyl (PFP) ester.

7. A siglec conjugate of formula (Ia): 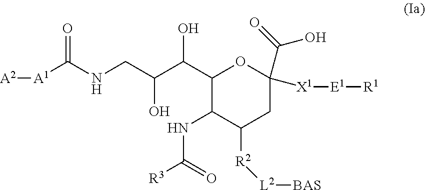
wherein:
A¹ is absent or —(CH₂)ₙ—, wherein n is an integer ranging from 1 to 10;
A² is alkyl, aryl, heteroaryl, cycloalkyl, or a substituted version thereof;
R¹ is H, alkyl, alkoxy, carboxyl, ester, amine, aryl, heteroaryl, cycloalkyl, heterocycle, carbohydrate, phosphate, squaramide, or a substituted version thereof;
R² is O, S, —NR⁴ R⁵, or —CR⁴ R⁵; wherein R⁴ and R⁵ are each independently selected from the group consisting of H, alkyl, substituted alkyl, alkenyl, substituted alkenyl, alkynyl, substituted alkynyl, cycloalkyl, substituted cycloalkyl, heterocyclyl, substituted heterocyclyl, aryl, substituted aryl, heteroaryl, and substituted heteroaryl;
R³ is OR^{3′}, NHR^{3′}, alkyl, —CH₂OR^{3′}, —CH₂NHR^{3′}, wherein R^{3′} is H, alkyl, aryl, heteroaryl, cycloalkyl, or a substituted version thereof;
X¹ is O, S, —CH₂—, —CH(OH)—, or triazole;
E¹ is absent, —(CH₂CH₂O)_{g}— or —(CH₂)_{g}—, wherein g is an integer ranging from 1 to 10;
L² is a linker; and
BAS is a biologically active substance;
a salt thereof, or a stereoisomer thereof.

8. A siglec conjugate of formula (IIa): 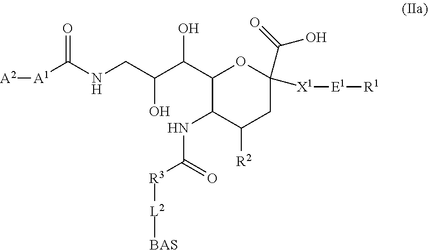
wherein:
A¹ is absent or —(CH₂)ₙ—, wherein n is an integer ranging from 1 to 10;
A² is alkyl, aryl, heteroaryl, cycloalkyl, or a substituted version thereof;
R¹ is H, alkyl, alkoxy, carboxyl, ester, amine, aryl, heteroaryl, cycloalkyl, heterocycle, carbohydrate, phosphate, squaramide, or a substituted version thereof;
R² is OH;
R³ is —O—, —NH—, —CH₂—, —CH₂O—, or —CH₂NH—;
X¹ is O, S, —CH₂—, —CH(OH)—, or triazole;
E¹ is absent, —(CH₂CH₂O)_{g}— or —(CH₂)_{g}—, wherein g is an integer ranging from 1 to 10;
L² is a linker; and
BAS is a biologically active substance;
a salt thereof, or a stereoisomer thereof.

9. A siglec conjugate of formula (IIIa): 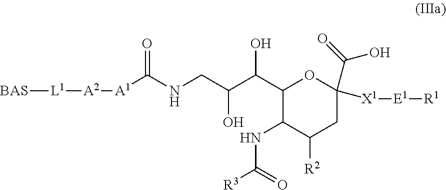
wherein:
A¹ is absent or —(CH₂)ₙ—, wherein n is an integer ranging from 1 to 10;
A² is alkyl, aryl, heteroaryl, cycloalkyl, or a substituted version thereof;
R¹ is H, alkyl, alkoxy, carboxyl, ester, amine, aryl, heteroaryl, cycloalkyl, heterocycle, carbohydrate, phosphate, squaramide, or a substituted version thereof;
R² is OH;
R³ is OR^{3′}, NHR^{3′}, alkyl, —CH₂OR^{3′}, —CH₂NHR^{3′}, wherein R^{3′} is H, alkyl, aryl, heteroaryl, cycloalkyl, or a substituted version thereof;
X¹ is O, S, —CH₂—, —CH(OH)—, or triazole;
E¹ is absent, —(CH₂CH₂O)_{g}— or —(CH₂)_{g}—, wherein g is an integer ranging from 1 to 10;
L² is a linker; and
BAS is a biologically active substance;
a salt thereof, or a stereoisomer thereof.

10. The siglec conjugate of claim 7, wherein the linker L² is represented by —(X²)ₘ—Z′, wherein:
each X² is independently selected from —(CH₂)ₕ—, —O—, —CH₂O—, —(CH₂CH₂O)ₕ—, —C(O)NH—, —C≡C—, —NH—C(O)—, squaramide, phosphamide, sulfonamide, and —CH₂C≡C—, triazole, carboxyl, ester, or substituted version thereof, wherein m is 0 or an integer ranging from 1 to 10, and h is an integer ranging from 1 to 10; and
Z′ is polyethylene glycol, amino, alkoxy, amide, triazole, ester, aryl, heteroaryl, succinimidyl-thioether, or a substituted version thereof.

11. The siglec ligand of claim 1, wherein:
A¹ is —CH₂—;
A² is aryl, or a substituted version thereof;
X¹ is O;
E¹ is —CH₂—;
R¹ is phenyl;
R² is O; and
R³ is —CH₂OH.

12. The siglec ligand of claim 2, wherein:
A¹ is —CH₂—;
A² is aryl, or a substituted version thereof;
X¹ is O;
E¹ is —CH₂—;
R¹ is phenyl;
R² is OH; and
R³ is —CH₂O—.

13. The siglec ligand of claim 3, wherein:
A¹ is —CH₂—;
A² is alkyl, aryl, alkoxy, amine, or a substituted version thereof;
X¹ is O;
E¹ is —CH₂—;
R¹ is phenyl;
R² is OH; and
R³ is —CH₂OH.

14. The siglec ligand of claim 3, wherein:
A¹ is —CH₂—;
A² is aryl;
X¹ is S;
E¹ is absent;
R¹ is substituted phenyl;
R² is OH; and
R³ is —CH₂OH.

15. The siglec conjugate of claim 7, wherein the BAS is a protein or an oligonucleotide.

16. The siglec conjugate of claim 15, wherein the protein is selected from the group consisting of an antibody, an enzyme, a chimeric protein, recombinant protein antigen, and a viral particle.

17. The siglec conjugate of claim 16, wherein the antibody is selected from the group consisting of a monoclonal antibody, a bispecific antibody, an scFv, a Fab, a camelid, or a nanobody.

18. The siglec conjugate of claim 16, wherein the antibody is selected from the group consisting of adalimumab, infliximab, cetuximab, natalizumab, moxetumomab pasudotox, atezolizumab, nivolumab, abciximab, Brentuximab, Certolizumab pegol, elotuzumab, benralizumab, vedolizumab, galcanezumab, rituximab, alemtuzumab, dupilumab, golimumab, obinutuzumab, tildrakizumab, erenumab, mepolizumab, tamucirumab, ranibizumab, ustekinumab, reslizumab, ipilimumab, alirocumab, belimumab, panitumumab, avelumab, necitumumab, mogamulizumab, olaratumab, brodalumab, eculizumab, pertuzumab, pembrolizumab, and tocilizumab.

19. The siglec conjugate of claim 15, wherein the protein is selected from the group consisting of erythropoietin, thrombopoietin, human growth hormone, tissue factor, IFNβ-1b, IFNβ-1a, IL-2 or the IL-2 mimetic aldesleukin, exenatide, albiglutide, alefacept, palifermin, and belatacept.

20. The siglec conjugate of claim 16, wherein the enzyme is selected from the group consisting of asparaginase Erwinia chrysanthemi, phenylalanine ammonia-lyase, alpha-galactosidase A, acid a-glucosidase (GAA), glucocerebrosidase (GCase), aspartylglucosaminidase (AGA), alpha-L-iduronidase, iduronate sulfatase, sulfaminase, a-N-acetylglucosaminidase (NAGLU), heparin acetyle CoA: α-glucosaminide N-acetyltransferase (HGSNAT), N-acetylglucosamine 6-sulfatase (GNS), N-glucosamine 3-O-sulfatase (arylsulfatase G or ARSG), N-acetylgalactosamine 6-sulfatase, beta-galactosidase, N-acetylgalactosamine 4-sulfatase, beta-glucuronidase, Factor VIII, Factor IX, palmitoyl protein thioesterase (PPT1), IdeS (protease), Endoglycosidase (EndoS) (glycosidase), and Urate oxidase/uricase, and Tripeptidyl peptidase (TPP1).

21. The siglec conjugate of claim 16, wherein the viral particle is selected from a recombinant adeno-associated virus (rAAV) particle, a recombinant human adenovirus (rHAdV) particle, a recombinant Herpes Simplex Virus (rHSV) particle, a recombinant papillomavirus (PV) particle, a recombinant polyomavirus particle, a recombinant vaccinia virus particle, a recombinant cytomegalovirus (CMV) particle, a recombinant baculovirus particle, a recombinant human papillomavirus (HPV) particle, and a recombinant retrovirus particle.

22. A pharmaceutical composition comprising:
a siglec conjugate according to claim 7; and
a pharmaceutical excipient.

23. A method of making a siglec conjugate, the method comprising:
covalently attaching a siglec ligand of claim 1 to a biologically active substance (BAS),
thereby making the conjugate.

24. A method of treating a patient for a condition, the method comprising:
administering to the patient a siglec conjugate according to claim 7.

25. The method of claim 24, wherein the condition is a chronic immune disease selected from the group consisting of rheumatoid arthritis, psoriatic arthritis, ankylosing spondylitis, Crohn's disease, ulcerative colitis, psoriasis, hidradenitis suppurativa, uveitis, and juvenile idiopathic arthritis, wherein the administering comprises administering to the individual an engineered hypoimmunogenic TNFα-specific antibody selected from adalimumab and infliximab in an amount effective to treat the chronic immune disease.

26. The method of claim 24, wherein the condition is leukemia, wherein the BAS is asparaginase from Erwinia chrysanthemi.

27. The method of claim 24, wherein the condition is multiple sclerosis, wherein the BAS is natalizumab, IFNβ-1b, or IFNβ-1a.

28. The method of claim 24, wherein the condition is an antibody response to transplanted tissue or an autoimmune indication, wherein the BAS is IdeS or EndoS.

29. The method of claim 24, wherein the condition is gout, wherein the BAS is urate oxidase/uricase.

30. The method of claim 24, wherein the transplanted tissue is an allogeneic graft.

31. The method of claim 24, wherein the transplanted tissue is a xenograft.

32. The method of claim 24, wherein the tissue is selected from kidney, heart, lung, liver, pancreas, trachea, vascular tissue, skin, bone, cartilage, adrenal tissue, fetal thymus, and cornea.

33. The method of claim 24, wherein the condition is Type 2 Diabetes and the BAS is exenatide or albiglutide.

34. The method of claim 24, wherein the condition is enzyme deficiency and the BAS is the deficient enzyme.

35. The method of claim 24, wherein the enzyme deficiency is a deficiency for an enzyme selected from the group consisting of phenylalanine ammonia-lyase (PKU), alpha-galactosidase A (for Fabry), acid α-glucosidase (GAA, for Pompe), glucocerebrosidase (GCase, for Gaucher), aspartylglucosaminidase (AGA, for Aspartylglucosaminuria), alpha-L-iduronidase (for MPS 1), iduronate sulfatase (for MPS II), sulfaminase (MPS IIIIa), α-N-acetylglucosaminidase (NAGLU, for MPS IIIB), heparin acetyle CoA: α-glucosaminide N-acetyltransferase (HGSNAT, for MPS IIIC), N-acetylglucosamine 6-sulfatase (GNS, for MPS IIID), N-glucosamine 3-O-sulfatase (arylsulfatase G or ARSG, MPS IIIE), N-acetylgalactosamine 6-sulfatase (for MPS IVA), beta-galactosidase (for MPS IVB), N-acetylgalactosamine 4-sulfatase (for MPS VI), beta-glucuronidase (for MPS VI), Factor VIII (for hemophilia A), Factor IX (for hemophilia B), palmitoyl protein thioesterase (PPT1, for CLN1), Tripeptidyl peptidase (TPP1, for CLN2), and cystathionine beta synthase (CBS) deficiency.

36. The method of claim 24, wherein the condition is a monogenic disease, wherein the BAS is a viral particle comprising a transgene encoding a therapeutic product

37. The method of claim 24, wherein the BAS is a biotherapeutic, wherein the method further comprises:
drawing serum from the individual 8 weeks after administering the biotherapeutic conjugate and assessing the serum for biotherapeutic-specific antibodies, wherein the titer of biotherapeutic-specific antibodies is 50% of the titer that would be elicited by a corresponding unengineered biotherapeutic.

38. The method of claim 24, wherein the titer of biotherapeutic-specific antibodies is 20% of the titer that would be elicited by a corresponding unengineered biotherapeutic.

39. The method of claim 24, wherein the titer of biotherapeutic-specific antibodies is 5% of the titer that would be elicited by a corresponding unengineered biotherapeutic.

40. The method of claim 24, wherein biotherapeutic-specific antibodies cannot be detected.
